(19) United States
(12) Patent Application Publication
(10) Pub. No.: US 2025/0197462 A1
(43) Pub. Date: Jun. 19, 2025

(54) COMPOSITIONS, SYSTEMS, AND METHODS FOR ACTIVATING AND SILENCING GENE EXPRESSION

(71) Applicant: The Board of Trustees of the Leland Stanford Junior University, Stanford, CA (US)

(72) Inventors: Michael C. Bassik, Stanford, CA (US); Joshua Tycko, Stanford, CA (US); Lacramioara Bintu, Stanford, CA (US)

(21) Appl. No.: 18/683,060

(22) PCT Filed: Aug. 17, 2022
(86) PCT No.: PCT/US2022/075082

§ 371 Date: Feb. 12, 2024

Related U.S. Application Data
(60) Provisional application No. 63/234,096, filed on Aug. 17, 2021.

(51) Int. Cl. C07K 14/47; A61K 48/00; C12N 15/11; C12N 15/85

(52) U.S. Cl. CPC C07K 14/4702; A61K 48/005; C12N 15/11; C12N 15/85; C07K 2319/71; C07K 2319/80

(57) ABSTRACT

Provided herein are compositions, systems, and methods for the generation, identification, and characterization of effector domains for activating and silencing gene expression. In particular, synthetic transcription factors comprising one or more transcriptional activator domains, one or more transcriptional repressor domains, or a combination thereof fused to a heterologous DNA binding domain, and methods of using thereof are provided.

### CROSS-REFERENCE TO RELATED APPLICATIONS

This application claims the benefit of U.S. Provisional Application No. 63/234,096, filed Aug. 17, 2021, the content of which is herein incorporated by reference in its entirety.

### STATEMENT REGARDING FEDERALLY SPONSORED RESEARCH

This invention was made with Government support under contract GM128947 awarded by the National Institutes of Health. The Government has certain rights in the invention.

### FIELD

Provided herein are compositions, systems, and methods for the generation, identification, and characterization of effector domains for activating and silencing gene expression. In particular, synthetic transcription factors comprising one or more of the effector domains and methods of using thereof are provided.

### SEQUENCE LISTING STATEMENT

The contents of the electronic sequence listing titled STDU2-39797-601.xml (Size: 1,411,208 bytes; and Date of Creation: Aug. 17, 2022) is herein incorporated by reference in its entirety.

### BACKGROUND

Previous efforts to engineer synthetic transcription factors have pulled activation and repressor domains from a small toolbox of previously discovered effector domains. New methods are needed to expand this toolbox.

### SUMMARY

Provided herein are compositions, systems, and method for the generation, identification, and characterization of effector domains for activating and silencing gene expression. In particular, high throughput systems are provided to discover and characterize effector domains. In some embodiments, provided herein is a high throughput approach to discover and characterize effector domains that greatly expands the toolbox. These domains satisfy a critical need to engineer enhanced synthetic transcription factors for applications in gene and cell therapy, synthetic biology, and functional genomics.

In some embodiments, the methods for identification of effector domains comprise: a) preparing a domain library comprising a plurality of nucleic acid sequences each configured to express a fusion protein comprising a protein domain linked to an inducible DNA binding domain; b) transforming reporter cells with the domain library, wherein a reporter cell comprises a two-part reporter gene comprising a surface marker and a fluorescent protein under the control of a strong promoter, wherein the two-part reporter gene is capable of being silenced by a putative transcriptional repressor domain following treatment with an agent configured to induce the inducible DNA binding domain; c) treating the reporter cells with the agent for a length of time necessary for protein and mRNA degradation in the cell; d) separating reporter cells based on presence or absence of the surface marker, the fluorescent protein, or a combination thereof; e) sequencing the protein domains from the separated reporter cells; f) calculating for each protein domain sequence a ratio of sequencing counts from reporter cells not having the surface marker, the fluorescent protein, or a combination thereof to sequencing counts from reporter cells having the surface marker, the fluorescent protein, or a combination thereof; and g) identifying protein domains as transcriptional repressor.

In some embodiments, the methods for identification of effector domains comprise: a) preparing a domain library comprising a plurality of nucleic acid sequences each configured to express a fusion protein comprising a protein domain linked to an inducible DNA binding domain; b) transforming reporter cells with the domain library, wherein the reporter cells comprises a two-part reporter gene comprising a surface marker and a fluorescent protein under the control of a weak promoter, wherein the two-part reporter gene is capable of being activated by a putative transcriptional activator domain following treatment with an agent configured to induce the inducible DNA binding domain; c) treating the reporter cells with the agent for a length of time necessary for protein and mRNA production in the cell; d) separating reporter cells based on presence or absence of the surface marker, the fluorescent protein, or a combination thereof; e) sequencing the protein domains from the separated reporter cells; f) calculating for each protein domain sequence a ratio of sequencing counts from reporter cells not having the surface marker, the fluorescent protein, or a combination thereof to sequencing counts from reporter cells having the surface marker, the fluorescent protein, or a combination thereof; and g) identifying protein domains as transcriptional activator.

In some embodiments, the methods further comprise stopping treatment of the reporter cells with the agent and repeating steps d-g one or more times. In some embodiments, steps d-g are repeated at least 48 hours after stopping treatment of the reported cells with the agent.

In some embodiments, each protein domain is less than or equal to 80 amino acids. In some embodiments, the protein domain is from a nuclear-localized protein. In some embodiments, the protein domain comprises amino acid sequences of the wild-type protein domains from nuclear-localized proteins. In some embodiments, the protein domain comprises mutated amino acid sequences of protein domains from nuclear-localized proteins.

In some embodiments, the inducible DNA binding domain comprises a tag.

In some embodiments, the methods further comprise measuring expression level of protein domains. In some embodiments, the expression level is determined by measuring a relative presence or absence of the tag on the DNA binding domain.

In some embodiments, the reporter cells are treated with the agent for at least 3 days. In some embodiments, the reporter cells are treated with the agent for at least 5 days. In some embodiments, the reporter cells are treated with the agent for at least 24 hours. In some embodiments, the reporter cells are treated with the agent for at least 48 hours.

In some embodiments, the protein domain is identified as a transcription repressor when log 2 of the ratio is at least two standard deviations from (e.g., higher than) the mean of a poorly expressed negative control.

In some embodiments, the protein domain is identified as a transcription activator when log 2 of the ratio is at least two standard deviations from (e.g., lower than) the mean of weakly expressing negative control.

Also provided herein are synthetic transcription factor comprising one or more transcriptional activator domains, one or more transcriptional repressor domains, or a combination thereof fused to a heterologous DNA binding domain. In some embodiments, at least one of the one or more transcriptional activator domains or at least one of the one or more transcriptional repressor domains comprises an amino acid sequence having at least 70% identity to any of SEQ ID NOs: 1-896. In some embodiments, at least one of the one or more transcriptional activator domains or at least one of the one or more transcriptional repressor domains comprises an amino acid sequence having at least 70% identity to any of SEQ ID NOs: 1346-1401.

In some embodiments, the one or more transcriptional activator domains, one or more transcriptional repressor domains, or a combination thereof are selected from those found in any of Tables 1 to 7.

In some embodiments, the synthetic transcription factor comprises two or more transcriptional activator domains or two or more transcriptional repressors domains fused to a heterologous DNA binding domain. In select embodiments, the synthetic transcription factor comprises three transcriptional activator domains or transcriptional repressors domains fused to a heterologous DNA binding domain.

In some embodiments, at least one of the one or more transcriptional activator domain comprises an amino acid sequence having at least 70% identity to any of SEQ ID NOs: 563-664. In some embodiments, at least one of the one or more transcriptional activator domain is selected from those found in Table 2.

In some embodiments, the one or more transcriptional activator domains comprises an amino acid sequence having at least 70% identity to any of SEQ ID NOs: 563, 564, 565, 566, 575, 576, 579, and 580. In some embodiments, the one or more transcriptional activator domains comprise an amino acid sequence having: SEQ ID NO: 563 or SEQ ID NO: 564; SEQ ID NO: 565 or SEQ ID NO: 566; SEQ ID NO: 575 or SEQ ID NO: 576; SEQ ID NO: 579 or SEQ ID NO: 580; or a combination thereof. In some embodiments, the one or more transcriptional activator domains comprises an amino acid sequence having two or more of: SEQ ID NOs: 563, 565, 575, and 579. In some embodiments, the one or more transcriptional activator domains comprises an amino acid sequence having SEQ ID NOs: 563, 565, and 579. In some embodiments, the one or more transcriptional activator domains further comprise an amino acid sequence of SEQ ID NO: 575.

In some embodiments, the synthetic transcription factor comprises an amino acid sequence having at least 70% identity to any of SEQ ID NOs: 1331-1344. In some embodiments, the synthetic transcription factor comprises an amino acid sequence of any of SEQ ID NOs: 1331-1344.

In some embodiments, the at least one of the one or more transcriptional repressor domain comprises an amino acid sequence having at least 70% identity to any of SEQ ID NOs: 1-562 and 665-896. In some embodiments, the at least one of the one or more transcriptional repressor domain comprises an amino acid sequence having at least 70% identity to any of SEQ ID NOs: 1346-1401. In some embodiments, the at least one of the one or more transcriptional repressor domain is selected from those found in any of Tables 1, 3, 4 or 6.

In some embodiments, the one or more transcriptional repressor domains comprise an amino acid sequence having at least 70% identity to any of SEQ ID NOs: 32, 36, 363, or a combination thereof. In some embodiments, the synthetic transcription factor comprises an amino acid sequence of SEQ ID NO: 1345.

In some embodiments, the one or more transcriptional activator domain or the one or more transcriptional repressor domain is identified by the methods disclosed herein.

In some embodiments, the heterologous DNA binding domain comprises a programmable DNA binding domain. In some embodiments, the DNA binding domain is derived from a Clustered Regularly Interspaced Short Palindromic Repeats associated (Cas) protein. In some embodiments, the DNA binding domain is derived from Transcription activator-like effectors (TALEs) domains. In some embodiments, the DNA binding domain is an inducible DNA binding system.

Also provided herein are nucleic acids encoding a synthetic transcription factor or an effector domain, as disclosed herein. In some embodiments, the nucleic acid in under control of an inducible promoter. In some embodiments, the nucleic acid in under control of a tissue specific promoter. In some embodiments, the nucleic acid encodes at least one additional transcription factor or effector domain.

Further provided herein is a composition or system comprising a synthetic transcription factor, a nucleic acid, a vector, or a cell as disclosed herein. In some embodiments, the composition comprises two or more synthetic transcription factors, nucleic acids, vectors, or cells. In some embodiments, the composition further comprises a guide RNA or a nucleic acid encoding a guide RNA.

Additionally, provided are methods of modulating the expression of at least one target gene in a cell. In some embodiments, the at least one target gene in a cell is an endogenous gene, an exogenous gene, or a combination thereof. The methods comprise introducing into the cell at least one synthetic transcription factor, nucleic acid, vector, or composition or system, as described herein. The gene expression of the at least one target gene is modulated when gene expression levels of the at least one target gene are increased or decreased compared to normal gene expression levels for the at least one target gene. In some embodiments, the synthetic transcription factor comprises a Cas protein DNA binding domain and the method further comprises contacting the cell with at least one guide RNA.

In some embodiments, the cell is in vitro (e.g., ex vivo) or in a subject.

In some embodiments, the gene expression of at least two genes are modulated.

### BRIEF DESCRIPTION OF THE DRAWINGS

FIGS. 1A-1G show high-throughput recruitment measures transcriptional repressor activity of thousands of Pfam-annotated domains from nuclear-localized proteins. FIG. 1A—Length of Pfam-annotated domains in human proteins that localize to the nucleus. Domains ≤80 amino acids were selected for inclusion in the library. FIG. 1B—Schematic of screen to identify transcriptional repressors. The repression reporter uses a strong pEF promoter that can be silenced by dox-mediated recruitment of repressor domains. The cells were treated with doxycycline for 5 days, ON and OFF cells were magnetically separated and the domains were sequenced. Dox was removed and additional time points were taken on Days 9 and 13. FIG. 1C—The reproducibility of log 2(OFF:ON) ratios from independently transduced biological replicates is shown and selected domain families are colored. FIG. 1D—Boxplots of top repressor domain families, ranked by the maximum repressor strength at day 5 of a domain within the family. FIG. 1E—Individual validation time course for hit RYBP domain, measured by flow cytometry. FIG. 1F—Additional validation time courses for a panel of repressor domains. Domain length is listed in parentheses, because some domains were tested as the exact 80 AA sequence from the library and some were tested as a shorter sequence trimmed to the region annotated as a domain by Pfam. 1000 ng/ml dox was added on day 0 and removed on day 5. FIG. 1G—Correlation of screen measurements with individual validation flow cytometry measurements for a collection of KRAB effector domains.

FIGS. 2A-2D show repressive KRAB domains are in younger KRAB-Zinc finger proteins that co-localize and bind to the KAP1 co-repressor. FIG. 2A—KRAB silencing function was compared with the KRAB Zinc Finger protein architecture that the domain is natively found in. FIG. 2B—KRAB silencing function was compared with the KRAB Zinc Finger gene evolutionary age, as determined by finding the most recent ortholog for the gene using its full DNA-binding zinc finger array sequence. FIG. 2C—KRAB domains were categorized as silencers or non-silencers and their genomic localization in ChIP-seq datasets was compared with the localization of the co-repressor KRAB-associated Protein 1 (KAP1). FIG. 2D-Repression strength distributions of KRAB domains categorized by whether their KRAB Zinc Finger gene interacts significantly with co-repressor KAP1 in a mass spec dataset. Dot colors are the quintile of the KRAB domain expression level.

FIGS. 3A-3G show a deep mutational scan of the ZNF10 KRAB domain identifies substitutions that reduce or enhance repressor activity. FIG. 3A—Deep mutational scanning library includes all single and consecutive double and triple substitutions in the KRAB domain from ZNF10. The DNA oligos are designed to be more distinct than the protein sequences by varying codon usage. Red residues differ from the WT sequence. FIG. 3B—All single and triple substitution variants' repressor measurements relative to the WT are shown underneath a schematic of the KRAB domain. Sequences: X-axis is SEQ ID NO: 1440 and all Y-axis sequences are SEQ ID NO: 1443. FIG. 3C—Average mutation effects on repression at Day 9, compared to sequence conservation from a multiple sequence alignment of all human KRAB domains (computed with ConSurf), FIG. 3D—Correlation of high-throughput measurements with previously published low-throughput data using the CAT assay in a different cell type. FIG. 3E—Individual time-courses of KRAB mutants validate the effects of substitutions in the A/B-boxes and N-terminus. FIG. 3F—For each position at each timepoint in FIG. 3B, the distribution of all single substitutions was compared to the distribution of wild-type effects (Wilcoxon rank sum test). Positions with signed log 10(p)<−5 at day 5 are colored in red (highly significantly decrease in silencing), with signed log 10(p)<−5 at day 9 but not day 5 are colored in green, and the position W8 with log 10(p)>5 at day 13 is colored in blue (highly significant increase). Dashed horizontal lines show the hit thresholds. The sequence conservation ConSurf score is shown in orange. FIG. 3G—Residues that abolish silencing at day 5 when mutated are mapped onto the ordered region of the NMR structure of mouse KRAB A-box (PDB: 1v65).

FIGS. 4A and 4B show that homeodomain repression strength is colinear with Hox gene organization. FIG. 4A—Ranking of homeobox gene families or classes by median repression strength at Day 5. The HOXL and NKL subclasses of the ANTP class homeodomains and the PRD and LIM classes, which contain the strongest homeodomain repressors, are split into individual gene families while the remaining classes are aggregated. Dot colors are the quintile of the Homeodomain expression level as measured in the HT-expression assay. FIG. 4B—Repressor strength at day 5 of the homeodomains from the Hox gene families. Arrows represent the genes found in the four human Hox loci and point in the direction of Hox gene transcription. Grey bars separate the gene families. Spearman's rho and the p-value were computed for the relationship between the gene number and repressor strength across all Hox genes. Data was filtered to remove any domains that had fewer than 10 counts in any of the Day 5 sequencing samples.

FIGS. 5A-5F show that high-throughput recruitment discovers activator domains, including a potent, acidic, and divergent KRAB domain variant in ZNF473. FIG. 5A—Schematic of the activation reporter which uses a weak minCMV promoter that can be activated by dox-mediated recruitment of activating domains, and a schematic of the activation screen. The pool of cells was treated with doxycycline for 48 hours, ON and OFF cells were magnetically separated with ProG Dynabeads and the domains were sequenced. FIG. 5B—The reproducibility of log 2(OFF:ON) ratios from independently transduced biological replicates is shown with known activator domain families (FOXO-TAD, Myb LMSTEN, TORC_C) colored. FIG. 5C—GO term enrichments of genes containing a domain with activation strength below a threshold. FIG. 5D—Activator domains (red) are more acidic than non-hits (grey). FIG. 5E—List of domain families, ranked by mean activation strength. FIG. 5F—KRAB domains were aligned and clustered by sequence, providing similar results to the classification in Helleboid 2019. The cluster of most divergent KRAB sequences is labeled variant KRABs in green. Results from screens are shown below in heatmaps. Standard KRABs function as repressors, if they are well-expressed. The variant KRABs show mixed effects as repressors, activators, and no transcriptional effect in the screens.

FIGS. 6A-6F show that a tiling library uncovers new autonomous repressor domains within large chromatin regulator proteins. FIG. 6A—Graphical depiction of library in which 80 AA tiles cover the protein sequence, with a 10 AA sliding window. FIG. 6B—The reproducibility of log 2(OFF:ON) ratios from independently transduced biological replicates is shown. FIG. 6C—Repression at Day 5 is compared with known domain architecture for the MGA protein. Two repressor domains are found outside the previously annotated regions. FIG. 6D—Flow cytometry time courses validate the individual MGA effectors as 80 AA tiles. FIG. 6E—The effectors were minimized to 10 and 30 AA subtiles by selecting the sequence shared in common among the tiles that show repressor activity in the screen. These minimized sequences were validated individually with flow cytometry time courses. FIG. 6F—Individual validation of additional 80 AA repressor hits from the tiling screen. rTetR-tile fusions were delivered to K562 reporter cells by lentivirus and cells were treated with 100 ng/ml dox for 5 days, then dox was removed. Cells were analyzed by flow cytometry and the fraction of cells OFF was measured by gating the cells by their citrine expression level.

FIGS. 7A-7D show that a recruitment assay measures gene silencing by lentiviral rTetR-domain fusions with a fluorescent reporter. FIG. 7A—Schematic of lentiviral vector. FIG. 7B—Pilot test in K562 reporter cells, showing citrine OFF:ON FACS histograms over time for ZNF10KRAB cloned onto pJT050. 1000 ng/ml dox was added on day 0 and removed on day 5. FIG. 7C—Fraction of cells ON over time FIG. 7D—The reporter system was also established in HEK293T cells. Cells were transfected with plasmid encoding rTetR-KRAB or pOri control and treated with or without 1000 ng/ml dox for 2 (top) and 4 (bottom) days before being analyzed with flow cytometry.

FIGS. 8A-8E show high-throughput measurements of domain expression by FLAG staining, sorting, and sequencing. FIG. 8A—Schematic of a high-throughput approach to measure the expression level for each domain fusion in the library. FIG. 8B—Reproducibility of domain expression measurements. FIG. 8C—Validation with Western Blot. FIG. 8D—Stability of sub-libraries-random are destabilized, tiles are similar to Pfam domains. FIG. 8E-Stability related to net charge of residue and residues which are classified as disorder promoting.

FIGS. 9A-9E show a screen of Pfam domains for repressor function. FIG. 9A—Flow cytometry of library of cells before and after magnetic separation. FIG. 9B—PANTHER protein class enrichments for stable vs transient repressors top 10, log P. FIG. 9C—Full list of domain families, ranked by repressor strength at day 5. FIG. 9D—TetR-SUMO fusions silence the reporter. Mutation in SUMO conjugation site (GG91AA) reduce silencing speed and mutation in SUMO-interacting non-covalent binding site reduces silencing memory. FIG. 9E—Validation of Domains of Unknown Function (DUFs) with repressor activity.

FIGS. 10A-10C show a KRAB deep mutational scan. FIG. 10A—OFF:ON scores from two biological replicates of a deep mutational library of the KRAB domains from ZNF10 at Day 5, 9 and 13. FIG. 10B—FLAG-tag stain for KRAB variant expression level: non-silencing one gets degraded. B-box mutants are stable. FIG. 10C—FLAG-tag stain correlates with FLAG-tag Western Blot.

FIGS. 11A-11C show activator screen data. FIG. 11A—Pilot test, electroporating rTetR-VP64 to K562 minCMV reporter cells. After doxycycline is added, the reporter cells turn ON as measured by flow cytometry for citrine expression. FIG. 11B—Magnetic separation of pooled library during activator screen, analyzed by flow cytometry. FIG. 11C—Comparison of HT-recruit transcriptional regulation measurements, using the Pfam domain library with two different reporter promoters. Each domain is a dot and the dot's size is the expression quartile as measured in the FLAG screen.

FIGS. 12A-12D show hundreds of repressors discovered in a screen of thousands of Pfam domains. FIG. 12A—Boxplots of top repressor domain families, ranked by the maximum repressor strength at day 5 of any domain within the family. Line shows the median, whiskers extend beyond the high- and low-quartile by 1.5 times the interquartile range, and outliers are shown with diamonds. Dashed line shows the hit threshold. Boxes colored for domain families highlighted in the text. FIG. 12B—Individual validations for RYBP domain and two Domains of Unknown Function (DUF) with repressor activity, measured by flow cytometry. Untreated cell distributions are shown in light grey and doxycycline-treated cells are shown in colors, with two independently-transduced biological replicates in each condition. The vertical line shows the citrine gate used to determine the fraction of cells OFF. FIG. 12C—Validation time courses fit with the gene silencing model: exponential silencing with rate ks, followed by exponential reactivation. Doxycycline (1000 ng/ml) was added on day 0 and removed on day 5 (N=2 biological replicates). The fraction of mCherry positive cells with the citrine reporter OFF was determined by flow cytometry, as in FIG. 12B, and normalized for background silencing using the untreated, time-matched controls. FIG. 12D—Correlation of high-throughput measurements at day 5 with the silencing rate ks (R²=0.86, n=15 domains, N=2-3 biological replicates). Horizontal error bars are the standard deviation for the fitted rates, vertical error bars are the range of screen biological replicates, and dashed lines are the 95% confidence interval of the linear regression.

FIGS. 13A-13E show Hox homeodomain repression strength is colinear with Hox gene organization and associated with positive charge. FIG. 13A—Ranking of homeobox gene classes by median repression strength of their homeodomain at day 5. Horizontal line shows the hit threshold. None of the 5 homeodomains from the CERS class were well-expressed. FIG. 13B—Homeodomains from the Hox gene families. (Top) Hox gene expression pattern along the anterior-posterior axis is colored by Hox paralog number on an adapted embryo image. Hox 11 and 12 are expressed both at the posterior end and along the proximal-distal axis of limbs. (Middle) Repression strength after 5 days of dox. Dots are colored by the Hox cluster and the paralog number is colored as in the embryo diagram. Spearman's rho and p-value were computed for the relationship between the paralog number and repressor strength across all Hox genes. (Bottom) Colored arrows represent the genes found in the four human Hox clusters and point in the direction of Hox gene transcription from 5′ to 3′. Grey bars separate gene sequence similarity groups as previously classified. FIG. 13C—Multiple sequence alignment of Hox homeodomains, with stronger repressors at the top (as ranked by OFF:ON ratio at day 5), showing the RKKR motif (SEQ ID NO: 1330) highlighted in red. Other basic residues within the N-terminal arm are colored in lavender. FIG. 13D—Correlation between the number of positively charged residues in the N-terminal arm upstream of Helix 1 of each Hox homeodomain and the average repression at day 5. Dot color shows paralog number. FIG. 13E-NMR structure of the HOXA13 homeodomain retrieved from PDB ID: 2L7Z, with RKKR motif (SEQ ID NO: 1330) highlighted in red. The sequence from G15 to S81, using the coordinates from the multiple sequence alignment, is shown.

FIGS. 14A-14G show discovery of activator domains. FIG. 14A—Schematic of the activation reporter which uses a weak minCMV promoter that can be activated by doxycycline-mediated recruitment of activating effector domains fused to rTetR. FIG. 14B—Reproducibility of high-throughput activator measurements from two independently transduced biological replicates. The pool of cells containing the activation reporter in (FIG. 14A) were transduced with the nuclear domain library and treated with doxycycline for 48 hours; ON and OFF cells were magnetically separated, and the domains were sequenced. The ratios of sequencing reads from the OFF vs. ON cells are shown for domains that were well-expressed. Pfam-annotated activator domain families (FOXO-TAD, Myb LMSTEN, TORC_C) are colored in shades of red. A line is drawn to the strongest hit, the KRAB domain from ZNF473. The hit threshold is a dashed line drawn two standard deviations below the mean of the poorly expressed domain distribution. FIG. 14C—Rank list of domain families with at least one activator hit. Families previously annotated as activators in Pfam are in red. The dashed line represents the hit threshold, as in FIG. 14B. Only well-expressed domains are shown. FIG. 14D—Acidity of effector domains from the Pfam library, calculated as net charge per amino acid. (Left) Comparison of the nonhit, well-expressed Pfam domains (except KRAB and annotated activators) with the activator hits. The Pfam-annotated activator domain families are shown as a group as a positive control (orange). (Right) Comparison of the activator hits and non-hits from the KRAB domain family. P-values from Mann-Whitney test shown with bars between compared groups. n.s.=not significant (p>0.05). FIG. 14E—Phylogenetic tree of all well-expressed KRAB domains with the sequence-divergent variant KRAB cluster shown in green (top). High-throughput recruitment measurements for repression at Day 5 are shown in blue (middle) and measurements for activation are shown in red (bottom). Dashed horizontal lines show hit thresholds. An example repressor KRAB from ZNF10, the repressor KRAB 1 from ZFP28, and all of the activator KRAB domains are called out with larger labels. The KRAB domain start position is written in parentheses. FIG. 14F—Individual validation of variant KRAB activator domains. rTetR(SE-G72P)-domain fusions were delivered to K562 reporter cells by lentivirus and selected for with blasticidin, cells were treated with 1000 ng/ml doxycycline for 2 days, and then citrine reporter levels were measured by flow cytometry. Untreated cell distributions are shown in light grey and doxycycline-treated cells are shown in colors, with two independently-transduced biological replicates in each condition. The vertical line shows the citrine gate used to determine the fraction of cells ON and the average fraction ON for the doxycycline-treated cells is shown. FIG. 14G—Distance of ChIP peak locations of KRAB Zinc Finger proteins away from the nearest peaks of the active chromatin mark H3K27ac. KRAB proteins are classified by their status as hits (blue) or non-hits (green) in the repressor screen at day 5 (left). In addition, data is shown individually for ZNF10 which contains a repressor hit KRAB (black), ZNF473 which contains an activator hit KRAB (red), and ZFP28 which contains both an activator hit and a repressor hit KRAB (yellow) (right). Each dot shows the fraction of peaks in a 40 basepair bin. ChIP-seq and ChiP-exo data retrieved from (ENCODE Project Consortium et al., 2020; Imbeault et al., 2017; Najafabadi et al., 2015; Schmitges et al., 2016). Only solo peaks, where a single KRAB Zinc Finger binds, are included for the aggregated data (blue and green dots, left), but all peaks are included for the individual proteins because the number of solo peaks is low for each individual protein (red, black, and yellow dots, right).

FIGS. 15A-15I show compact repressor domains discovered within nuclear proteins. FIG. 15A—Schematic of 80 AA tiling library covering a curated set of 238 nuclear-localized proteins. These tiles were fused to rTetR and recruited to the reporter, using the same workflow as in FIG. 1 to measure repression strength. FIG. 15B—Tiled genes ranked by maximum repressor function at day 5 shown with a dot for each tile. Hits are tiles with a log 2(OFF:ON)≥2 standard deviations above the mean of the negative controls. Genes with a hit tile are colored in a gradient and genes without any hit tiles are colored in grey. FIG. 15C—Tiling CTCF. Diagram shows protein annotations retrieved from UniProt. Horizontal bars show the region spanned by each tile and vertical error bars show the standard error from two biological replicates of the screen. The strongest hit tile is highlighted with a vertical gradient and annotated as a repressor domain (orange). FIG. 15D—Tiling BAZ2A (also known as TIP5). FIG. 15E—Individual validations. Lentiviral rTetR(SE-G72P)-tile fusions were delivered to K562 reporter cells, cells were treated with 100 ng/ml doxycycline for 5 days (between dashed vertical lines), and then doxycycline was removed. Cells were analyzed by flow cytometry, the fraction of cells with citrine reporter OFF was determined and the data fit with the gene silencing model (N=2 biological replicates). Two KRAB repressor domains are shown as positive controls. The tiling screen data that corresponds to the validations shown on the bottom (blue curves) is shown in FIG. 22. FIG. 15F—Tiling MGA. Two repressor domains are found outside the previously annotated regions and labeled as Repressor 1 and 2 (dark red, purple). The minimized repressor regions at the overlap of hit tiles are highlighted with narrow red vertical gradients. FIG. 15G—The maximal strength repressor tiles from two peaks in MGA were individually validated with the method described in FIG. 15E (N=2 biological replicates). FIG. 15H—The MGA repressor 1 sequence was minimized by selecting the region shared in common between all hit tiles in the peak, shown between dashed vertical lines and shaded in red. The protein sequence conservation ConSurf score is shown below with an orange line and the confidence interval (the 25th and 75th percentiles of the inferred evolutionary rate distribution) is shown in grey. The asterisks mark residues that are predicted to be functional (highly conserved and exposed) by ConSurf. The repressor 2 sequence was minimized with the same approach and also overlaps a region with predicted functional residues (data not shown). FIG. 15I—The MGA effectors were minimized to 10 and 30 AA sub-tiles, as shown in FIG. 15H, cloned as lentiviral rTetR(SE-G72P)-tile fusions, and were delivered to K562 reporter cells. After selection, cells were treated with 100 or 1000 ng/ml doxycycline for 5 days and the percentages of cells with the Citrine reporter silenced were measured by flow cytometry (N=2 biological replicates).

FIGS. 16A-16C show validation of lentiviral recruitment assay and dual reporter for gene silencing. FIG. 16A—Schematic of lentiviral recruitment vector with Golden Gate cloning site for creating fusions of effector domains to the dox-inducible DNA-binding domain rTetR. The constitutive pEF promoter drives expression of the rTetR-effector fusion and mCherry-BSD (Blasticidin S deaminase resistance gene), separated by a T2A self-cleaving peptide. FIG. 16B—(Top) Schematic of rTetR-KRAB fusion recruitment to the dual reporter gene. The reporter is integrated in the AAVS1 locus by TALEN-mediated homology-directed repair and the PuroR resistance gene is driven by the endogenous AAVS1 promoter. The dual reporter consists of a synthetic surface marker (Igκ-hIgG1-Fc-PDGFRβ) and a citrine fluorescent protein. (Bottom) Pilot test in K562 reporter cells. Reporter cells were generated by TALEN-mediated homology-directed repair to integrate the reporter into the AAVS1 locus and then selected with puromycin. Cells were then spinfected with lentivirus to deliver rTetR-KRAB, and then either left untreated or treated with 1000 ng/ml doxycycline to induce rTetR binding to DNA at the TetO sites. Untreated cell distributions are shown in light grey and doxycycline-treated cells are shown in black or orange, with two independently-transduced biological replicates in each condition. The lentivirus-treated cells are gated on mCherry as the delivery marker. The KRAB domain from human ZNF10 was used. FIG. 16C-Demonstration of magnetic separation of OFF from ON cells using ProG Dynabeads that bind to the synthetic surface marker. Ten million cells were subjected to magnetic separation using 30 μl of beads, and the citrine reporter expression was measured before and after by flow cytometry. Illustration of mixed ON and OFF cells being subjected to magnetic separation is shown on the right.

FIGS. 17A-17F show high-throughput measurements of domain expression by FLAG staining, sorting, and sequencing. FIG. 17A—(Top) Schematic of high-throughput strategy for measuring the expression level of each domain in the library. Domains under 80AA long are extended on both sides, using their native protein sequence, to reach 80AA so all synthesized library elements are the same length. (Middle) The library is cloned into a FLAG-tagged construct and delivered to K562 cells by lentivirus at low multiplicity of infection, such that the majority of cells express a single library member. The mCherry-BSD fusion protein enables blasticidin selection and a fluorescent marker for delivery and selection efficiency, without the use of a second 2A component. (Bottom) Expression is measured by staining the cells with anti-FLAG, sorting high and low expression populations, sequencing the domains, and computing the log 2(FLAGhigh:FLAGlow) ratio. FIG. 17B—Distribution of FLAG staining levels measured by flow cytometry before and after sorting into two bins (N=2 biological replicates of the cell library shown with overlapping shaded areas). FIG. 17C—Reproducibility of biological replicates from the domain expression screen (r²=0.82). Well-expressed domains, above the threshold (dashed line one standard deviation above the median of the random controls), were selected for further analysis in the transcriptional regulation screens. FIG. 17D—Validation of expression level for a panel of KRAB domains. Individual rTetR-3×FLAG-KRAB constructs were delivered to K562 cells by lentivirus. Cells were selected with blasticidin and confirmed to be >80% mCherry positive by flow cytometry. Expression level was measured by Western blot with anti-FLAG antibody. Anti-histone H3 was used as a loading control for normalization. Levels were quantified using ImageJ. FIG. 17E—Comparison of high-throughput measurements of expression with Western blots protein levels. These 6 KRAB domains were cloned individually using the exact 80 AA sequence from the Pfam domain library. FIG. 17F-Distribution of expression levels for different categories of library members. Random controls are poorly expressed compared to tiles across the DMD protein or Pfam domains (p<1e-5, Mann Whitney test). Dashed line shows the threshold for expression level, as in FIG. 17C.

FIGS. 18A-18K show identification of domains with repressor function. FIG. 18A—Flow cytometry shows citrine reporter level distributions in the pool of cells expressing the Pfam domain library, before and after magnetic separation using ProG DynaBeads that bind to the synthetic surface marker. Overlapping histograms are shown for two biological replicates. The average percentage of cells OFF is shown to the left of the vertical line showing the citrine level gate. 1000 ng/ml doxycycline was added on Day 0 and removed on Day 5. FIG. 18B—PANTHER protein class enrichments for nuclear proteins that contain repressor domains with stronger or weaker memory, when compared to the background set of all nuclear proteins with domains included in the library. FIG. 18C—rTetR-SUMO validation time courses fit with gene silencing model. The 80 AA sequence centered around the Rad60-SLD domain of SUMO3 and the trimmed domain were individually cloned into lentivirus and delivered to the reporter cells. 1000 ng/ml doxycycline was added on day 0 and removed on day 5 (N=2 biological replicates). The fraction of mCherry-positive cells with the citrine reporter OFF was determined by flow cytometry and normalized for background silencing using the untreated, time-matched controls. FIG. 18D—HUSH complex member MPP8 Chromo domain validation with the full 80 AA sequence used in the screen and sequences trimmed to match the Pfam and UniProt annotations. FIG. 18E—CBX1 Chromoshadow domain validation with 52 AA sequence trimmed to match the Pfam annotation. FIG. 18F—Polycomb 1 component SCMH1 SAM1 domain (also known as SPM) validation with 65 AA sequence trimmed to match the Pfam annotation. FIG. 18G—HERC2 Cyt-b5 domain validation with the full 80 AA sequence used in the screen and a 72 AA sequence trimmed to match the Pfam annotation. FIG. 18H—BIN1 SH3_9 domain validation. FIG. 18I—Polycomb 1 component PCGF2 zf-C3HC4_2 domain validation with 39 AA sequence trimmed to match the Pfam annotation. FIG. 18J—TOX HMG box domain validation with the full 80 AA sequence used in the screen and a 68 AA sequence trimmed to match the Pfam annotation. FIG. 18K-Validation of a random 80 AA sequence that functions as a repressor.

FIGS. 19A-19D show rTetR(SE-G72P) mitigates leaky KRAB silencing in human cells. FIG. 19A—Silencing by rTetR-KRAB fusions, showing leaky silencing without doxycycline treatment for a subset of KRAB domains (high gray bars). Constructs were delivered to reporter cells by lentivirus at day 0, cells were selected with blasticidin between days 3 and 11, cells were split into a doxycycline-treated or untreated condition at day 11, and reporter levels were measured by flow cytometry at day 16. Results are shown after gating for the mCherry positive cells. The KRAB domains were selected from three categories based on their measurements in the screen, labeled on the right. The bar shows the average and the error bar shows the standard deviation (N=3 independently transduced biological replicates). FIG. 19B—Leakiness can be mitigated by using rTetR(SE-G72P) or introducing 3×FLAG between rTetR and the KRAB domain from ZNF823. Constructs were delivered to reporter cells by lentivirus at day 0, cells were split into a doxycycline-treated or untreated condition at day 4, and reporter levels were measured by flow cytometry at day 7. Results are shown after gating for the mCherry positive cells. A non-leaky KRAB domain from ZNF140 was used as a control. The bar shows the average and the error bar shows the standard deviation (N=2 independently transduced biological replicates). FIG. 19C—The K562 reporter cell lines with stable lentiviral expression of either a leaky KRAB domain from ZNF823 or a non-leaky repressor KRAB domain from ZNF140 cloned as fusions with either rTetR or rTetR(SE-G72P) were treated with varied doses of doxycycline. Reporter levels were measured by flow cytometry four days later, and the percentage of mCherry positive cells with the citrine reporter OFF is shown (N=2 independently transduced biological replicates). The dose response was fit by least squares with a non-linear variable slope sigmoidal curve using PRISM statistical analysis software. FIG. 19D—Silencing and memory dynamics for all individual validations of KRAB domains, fit with the gene silencing model. rTetR(SE-G72P)-KRAB fusions were delivered to K562 reporter cells by lentivirus, selected with blasticidin, and then 10 ng/ml doxycycline was added on day 0 and removed on day 5 (N=2 biological replicates). The fraction of mCherry positive cells with the citrine reporter OFF was determined by flow cytometry and normalized for background silencing using the untreated, time-matched controls. 10 ng/ml dox was used to work in a dynamic range where it is easier to measure differences in silencing and memory capabilities between fast KRAB silencing domains. With 1000 ng/ml doxycycline, all of the repressor hit KRAB domains (greens and oranges) fully silence the reporter within 5 days with indistinguishable dynamics (data not shown). Notably, the KRABs that were leaky on rTetR (oranges), do not show significantly different memory dynamics from the KRABs that were not leaky (greens) when fused to the rTetR(SE-G72P). Importantly, none of the rTetR(SE-G72P)-KRAB fusions showed significant leaky silencing in the untreated condition.

FIGS. 20A-20H show deep mutational scan of ZNF10 KRAB used in CRISPRi. FIG. 20A—Flow cytometry shows citrine reporter levels in the cells with the pooled KRAB library, before and after magnetic separation using ProG DynaBeads that bind to the synthetic surface marker. Overlapping histograms are shown for two biological replicates. The average percentage of cells OFF is shown to the left of the vertical line showing the citrine level gate. FIG. 20B—OFF:ON enrichments from two biological replicates of the deep mutational library of the ZNF10 KRAB domain at days 5, 9 and 13. Cells were treated with 1000 ng/ml doxycycline for the first 5 days. Grey diagonal lines show where the average log 2(OFF:ON) is the median of the WT domains (black dots). The black diagonal lines show the fit linear model. FIG. 20C—Alignment of human ZNF10 KRAB with mouse KRAB used in the NMR structure (PDB: 1v65) and KRAB-O used in the recombinant protein binding assays (Peng et al., 2009). The ordered region is used in FIG. 3 and the aligned region containing all 12 necessary residues is used in (FIG. 20D). The residues necessary for silencing at day 5 are colored in red in the ZNF10 and PDB: 1v65 sequences. The residues necessary for binding recombinant KAP1 are colored in red and the residues unnecessary for binding KAP1 are colored in grey in the KRAB-O sequence, summarizing previously published results (Peng et al., 2009). FIG. 20D—Ensemble of 20 states of the KRAB NMR structure (PDB: 1v65). The residues necessary for silencing at day 5 are colored in red. FIG. 20E—Silencing and memory dynamics for all individual validations of KRAB ZNF10 mutants, fit with the gene silencing model. (Top) rTetR-KRAB fusions were delivered to K562 reporter cells by lentivirus, selected with blasticidin, and then 1000 ng/ml doxycycline was added on day 0 and removed on day 5 (N=2 biological replicates. (Bottom) rTetR(SE-G72P)-KRAB fusions were delivered to K562 reporter cells by lentivirus, selected with blasticidin, and then 10 ng/ml doxycycline was added on day 0 and removed on day 5 (N=2 biological replicates). The column labels describe the variant location within the KRAB domain and impact on effector function. The fraction of mCherry positive cells with the citrine reporter OFF was determined by flow cytometry and normalized for background silencing using the untreated, time-matched controls. All of the rTetR(SE-G72P)-KRAB fusions were also measured over 5 days of treatment with 1000 ng/ml doxycycline and the results were indistinguishable from those with rTetR, with all KRAB variants completely silencing the reporter except the EEW25AAA variant that does not silence (data not shown). FIG. 20F—Correlation of rTetR-KRAB fusion expression level and day 13 silencing score, from the Pfam domain library. Only KRAB domains that were shown to interact with co-repressor KAP1 by IP/MS (Helleboid et al., 2019) are included. FIG. 20G—Correlations of amino acid frequency with domain expression level, across the library of Pfam domains and controls (Pearson's r value is shown). FIG. 20H—Western blot for FLAG-tagged rTetR-KRAB fusions after lentiviral delivery to K562. Cells were selected for delivery with blasticidin and were confirmed to be >80% mCherry positive by flow cytometry. Expression level relative to the H3 loading control was quantified using ImageJ.

FIGS. 21A-21C show HT-recruit to a minimal promoter discovers activator domains. FIG. 21A—Flow cytometry for pooled library of Pfam domains in activation reporter cells, before and after magnetic separation. The percentage of cells ON is shown to the right of the citrine level gate, drawn with a vertical line. 1-2 biological replicates are shown with overlapping shaded areas. FIG. 21B—GO term enrichment of genes containing a hit activation domain, compared to the background set of all proteins containing a well-expressed domain in the library after counts filtering. Raw p-values are shown, and all shown terms are below a 10% false discovery rate. FIG. 21C—Individual validations of activator domains. rTetR(SE-G72P)-domain fusions were delivered to K562 reporter cells by lentivirus and selected with blasticidin. Cells were treated with 1000 ng/ml doxycycline for 2 days, and then citrine reporter levels were measured by flow cytometry. Untreated cell distributions are shown in light grey and doxycycline-treated cells are shown in colors, with two independently-transduced biological replicates in each condition. The vertical line shows the citrine gate used to determine the fraction of cells ON, and the average fraction ON for the doxycycline-treated cells is shown. VP64 is a positive control. Each domain was tested as both the extended 80 AA sequence from the library or the trimmed Pfam-annotated domain sequence, with the exceptions of Med9 and DUF3446 which had minimal extensions because the Pfam annotated regions are 75 and 69 AA, respectively. The corresponding results for the 80 AA library sequence for the KRAB domains are shown in FIG. 14.

FIGS. 22A-22H show identification of compact repressor domains in nuclear proteins with tiling screen. FIG. 22A—Flow cytometry shows citrine reporter level distributions in the pool of cells expressing the tiling library, before and after magnetic separation using ProG DynaBeads that bind to the synthetic surface marker. Overlapping histograms are shown for two biological replicates. The average percentage of cells OFF is shown to the left of the vertical line showing the citrine level gate. 1000 ng/ml doxycycline was added on Day 0 and removed on Day 5. FIG. 22B—High-throughput recruitment measurements from two biological replicates of a nuclear protein tiling library at Day 5 of doxycycline treatment and Day 13, 8 days after doxycycline removal. The hit calling threshold is 2 standard deviations above the mean of the random and DMD tiling controls. FIG. 22C—Tiling results for KRAB Zinc finger proteins ZNF57 and ZNF461. Each bar is an 80 AA tile and the vertical error bars are the range from 2 biological replicates. Protein annotations are sourced from UniProt. FIG. 22D—Tiling RYBP. Diagram shows protein annotations, retrieved using the UniProt ID written at top. Vertical error bars show the standard error from two biological replicates. FIG. 22E—Tiling REST. FIG. 22F—Tiling CBX7. FIG. 22G—Tiling DNMT3B. FIG. 22H (Top) Tiling DMD. (Bottom) Dynamics of silencing and memory after recruitment of DMD hit tiles. Cells were treated with 1000 ng/ml doxycycline for the first 5 days and citrine reporter levels were measured by flow cytometry. The percentage of cells OFF was normalized to account for background silencing and the data (dots) were fit with a gene silencing model (curves) (N=2 biological replicates).

FIGS. 23A-23B show activation of endogenous CD2 with new activator domains fused to dCas9.

FIG. 24 is schematic of compact human activator domains for tunable AAV gene therapy (left) and application using an inducible DNA binding domain constructs (right).

FIG. 25 shows that enhanced KRAB variant improves CRISPRi efficiency.

FIG. 26 shows that combinations of repressors can enable stronger silencing.

FIG. 27 shows that RYBP+MGA1+2 fusion deposits heterochromatin with similar efficiency as enhanced KRAB.

FIG. 28 shows mapping effector function across promoters and cell type contexts.

FIG. 29 shows that effector function can depend on the recruitment context.

FIG. 30 is a schematic of high-throughput domain screens to expand the CRISPR toolbox.

FIG. 31 is a schematic summary of effector functions.

FIG. 32 is a schematic of screens for effector dependencies.

FIG. 33 shows that MGA and KRAB have some shared top hits.

FIG. 34 shows that repressors are shared across K562 and HEK293T cells.

FIG. 35 shows effector function in stable cells lines with diverse reporter promoters.

FIG. 36 shows that most Pfam domains are expressed at similar levels when fused onto either rTetR or dCas9.

FIG. 37 is a graph of rTetR-Pfam library fusion expression at similar levels in two different cell types. The discrepant domains are not likely random noise as a group of KRAB domains show higher expression in K562 than HEK293T cells and a group of C2H2 ZF domains shown higher expression in HEK293T than K562 cells.

FIG. 38 is a graph of the correlation of repressor strength upon recruitment to PGK or UbC promoter reports.

FIG. 39 are graphs showing that the pEF promoter is silenced by more moderate non-KRAB repressors than PGK or UbC.

FIG. 40 are graphs showing that minimal promoters largely respond similarly to activators.

FIG. 41 is a schematic showing that some domains or combinations thereof are not leaky

FIGS. 42A-24D show that compact activators improve CRISPRa and inducible systems. FIG. 42A shows the ranking of activators in HT-recruit screens across target, cell-type, and DNA binding domain (DBD) contexts. Activators that were hits in at least two samples and had greater than 5 counts in both separated populations are included. The rho values for these activators were normalized in each sample, and the Z-scores are shown. The rows are clustered in an unbiased manner. NCOA3 (N), FOXO3 FOXO-TAD (F), and ZNF473 KRAB (Z) are labeled. n=2 replicates per rTetR screen (shown as columns) and n=1-2 replicates per sgRNA for dCas9 screens. FIG. 42B shows a comparison at the single cell level of CD2 activation and dCas9-activator delivery (BFP), after gating for CD2 sg717 delivery (GFP). Color shows density of cell events and smoothing is applied. The percentage of BFP+ cells is labeled (n=2 infections). FIG. 42C shows rapamycin-inducible expression of reporter citrine gene with ZFHD1 recruitment of NFZ or p65+HSF1, a previously described activator combination. Plasmids were transfected into HEK293T cells, one day later 10 nM rapamycin was added, and two days later citrine mean fluorescence intensity (MFI) was measured by flow cytometry (n=2 transfection replicates, bar shows mean and error bar shows standard deviation). FIG. 42D shows rapamycin-inducible expression of HGF with ZFHD1 recruitment of NFZ, after transfection in HEK293T cells. Secreted HGF protein concentrations were measured in the cell culture supernatant by ELISA after 2 days of rapamycin treatment with varied doses. Transfection of no plasmid and a constitutive pEF1a-HGF plasmid served as a negative and positive control, respectively (n=2 transfection replicates).

FIGS. 43A-43M show the characterization of transcriptional control tools using compact human activators. FIG. 43A is a graph of CRISPRa delivery 2 days after plasmid transfection in HEK293T cells measured by flow cytometry for the BFP marker on the dCas9-effector-T2A-BFP-P2A-BlastR transcript, with no gating on co-transfected delivery markers. dCas9-NFZ is significantly better delivered than dCas9-VPR with a greater fraction of BFP+ cells using a linear gate at 1.5e7 (P<0.05, two-tailed t-test). Each dot is an individual co-transfection of 500 ng of dCas9 plasmid and 300 ng of an sgRNA plasmid in a 24-well plate (n=11 transfection replicates for NFZ and VPR) or an untransfected negative control. Bar shows mean and error bars show standard deviation. FIG. 43B shows CRISPRa transcript expression level in the same flow cytometry data as in FIG. 43A, after accounting for overall delivery efficiencies by gating for transfectable cells based on the presence of GFP which is found on the co-transfected sgRNA plasmid. Each line is an individual co-transfection or the untransfected control and the black line shows the linear gate for BFP⁺ cells. FIG. 43C is a comparison of dCas9-NFZ and VPR fusions targeting CD2, CD20, CD28 surface marker genes in K562 cells. sgRNAs were first installed by lentiviral delivery and puromycin selection. Then dCas9 plasmids were electroporated. Two days later, cells were immunostained for CD2 (APC), CD20 (APC), or CD28 (PE) expression and analyzed by flow cytometry after gating for dCas9 (BFP) the stably expressed sgRNA (GFP). Each dot represents a different sgRNA targeting the gene (n=3-4 per gene). FIG. 43D is a graph of CRISPRa delivery to J774 macrophages by lentiviral infection with or without 4× LentiX concentration. FIG. 43E shows effect on the tripartite activator by changing the N, F, Z domains' orientation. The various configurations were fused onto dCas9 and targeted to the CD2 gene in K562 cells. Activation was measured by immunostaining CD2 with APC-conjugated antibodies followed by flow cytometry and the average percentage of cells ON is shown. The darker shaded histogram is CD2 sg717 and the lighter shade is sg718. FIG. 43F shows dCas9-activator delivery to K562 cells by lentiviral infection. 1 mL of unconcentrated lentivirus was infected and flow cytometry for BFP delivery marker was performed 3 days after infection. In FIG. 43G, 1 mL of 5× LentiX-concentrated lentivirus was used to infect cells and cells were analyzed by flow cytometry at 3 timepoints. Blasticidin selection was initiated on day 5. Each dot is an independent infection (n=8 replicates per dCas9 construct). FIG. 43H dCas9-activators were delivered to MCF10a cells by 10× concentrated lentivirus and delivery was measured 4 days later by flow cytometry. In FIG. 43I, rTetR-NZF, or VP64 as a control, were delivered to HEK293T reporter cells by lentivirus. After two days of dox to induce recruitment, activation and memory were measured throughout 8 days post-recruitment by flow cytometry. Means are from three biological replicates. Flow histograms are shown on the right. FIG. 43J is data from the fusion of fusion of SMARCA2 QLQ (Q) to dCas9-NZF. Two days after lentiviral delivery of dCas9-activators, K562 cells were immunostained with CD2 antibody to measure gene activation by flow cytometry, and the average percentage of cells ON is shown. The darker shaded histogram is CD2 sg717 and the lighter shade is sg718. In FIG. 43K, dCas9-QNZF was delivered to K562 cells by lentivirus and selected for with blasticidin, then sgRNAs were delivered by lentivirus and selected for with puromycin, then 8 days after sgRNA delivery the cells were stained for the targeted surface marker genes and measured by flow cytometry on the BioRad ZE5. Surface marker expression is shown after gating for dCas9 with BFP and sgRNA with GFP. Top: the darker shaded histogram is CD2 sg42 and the lighter shade is sg46; middle: the darker shade is CD20 sg135 and the lighter shade is sg148; bottom: the darker shade is CD28 sg56 and the lighter shade is sg94. FIG. FIG. 43L shows the percentage of CD2 endogenous gene activated 3 days after transient transfection of dCas9-activators and an sgRNA in HEK293T cells. Cells were immunostained for CD2 (APC) expression and analyzed by flow cytometry after gating for transfection (GFP on the sgRNA plasmid). Each dot represents an independently transfected biological replicate (n=2). FIG. 43M shows homotypic combination of Q, N, Z, F activators fused onto dCas9 and delivered stably by lentivirus to target CD2 in K562 cell lines stably expressing the sgRNA. The mean fluorescence intensity (MFI) of CD2 staining (Alexa 647) of the cell population after gating for delivery of the sgRNA (GFP) and dCas9 fusion (BFP) is shown. Staining was performed 9 days after dCas9 fusion infection. Each point is an sgRNA (sg717 or sg718) and bar shows the mean of two different sgRNAs.

FIGS. 44A and 44B show compact human activators for inducible AAV gene therapy. FIG. 44A is a schematic showing the human hepatocyte growth factor (HGF) gene in the rapamycin inducible AAV vector. FIG. 44B shows induction of HGF in HEK293T cells with rapamycin. Cells were grown at higher density and demonstrated an increase in HGF levels while minimizing background activation of the payload.

FIGS. 45A and 45B show CRISPR HT-recruit targeting an essential enhancer of GATA1. FIG. 45A shows CRISPR HT-recruit used to identify repressor domains by targeting an enhancer of the essential gene GATA1 and using growth as a selection strategy. dCas9-domain library fusions were targeted to the essential GATA1 gene with a guide against the TSS or two known GATA1 enhancers, eGATA1 and eHDAC6. The plasmid library was used to generate lentivirus to infect cells, and after 13 days of growth, gDNA was extracted and domains were sequenced. Growth phenotypes were quantified by comparing the domain counts at the final time point (D13) relative to the initial plasmid pool. FIG. 45B shows a comparison of CRISPR HT-recruit growth phenotypes using two guides: a safe-targeting sgRNA (N4293) and an eGATA1 enhancer-targeting sgRNA. Each black dot represents a Pfam domain, whereas blue dots represent KRAB domains. A low, more negative log₂(plasmid:Day 13) value for the safe-targeting sgRNA is associated with cell toxicity. Repression of eGATA1 is measured by log₂(plasmid:Day 13) such that domains with low, negative values represent strong repressors.

FIGS. 46A-46C show CRISPR interference with a strong ZNF705F KRAB domain is improved across target, cell-type, and DNA binding domain contexts. FIG. 46A shows KRAB domains ranked by repression strength in HT-recruit screens across target, cell type, and DBD contexts, with the top repressor KRAB at the top. Rows are ordered by average rank across all screens. KRAB domains from select proteins are labeled on the right. The rank is the average from two biological replicates for each screen, except the GATA1 screens with one replicate each. The HEK293T pEF screen has two timepoints: a silencing measurement at 4 days after dox addition, and a memory measurement at day 12 (8 days after dox removal). FIG. 46B is a CRISPRi benchmarking screen for KRAB repressors. A library with 405 guides targeting 37 essential genes was delivered into K562 cell lines that stably express a dCas9-repressor fusion, cells were passaged for 14 days, then the guides were sequenced to measure fitness effects (shown as a log₂ fold-change from the original plasmid pool to the final day 14 measurement from genomic DNA). Greater depletion is a measure of stronger silencing of the essential genes. Each dot shows the average effect for an sgRNA and the error bars show the S.D. from 2 screen replicates (n=405 sgRNAs). The diagonal line represents identity between KRAB domains. FIG. 46C shows dCas12a recruitment of ZNF10 and ZNF705F KRAB with guide RNAs to target CD43. dCas12a fusions and then guide RNAs were delivered by lentivirus to K562 cells. 9 days after guide RNA infection, cells were stained for surface marker expression and analyzed by flow cytometry, with gates applied for guide RNA expression (mCherry) and dCas12a expression (HA-tag stain). Infection replicates are shown as separate histograms and their average percentage of silenced cells is shown. Each line represents a biological replicate (n=2).

FIGS. 47A-47J show KRAB mutant and paralogs with improved CRISPRi efficiency. FIG. 47A is Western blot analysis of dCas9 ZNF10 KRAB and enhanced KRAB [WSR7EEE] mutant fusions. The dCas9-repressors were tagged with 3×FLAG to allow for probing with anti-FLAG antibodies. The band intensity ratio of FLAG to β-actin staining is shown below and used to quantify protein stability. dCas9 only was included as a control. FIG. 47B shows recruitment of dCas9 ZNF10 KRAB and WSR7EEE mutant fusions to the endogenous surface marker gene CD43 in K562 cells. sgRNAs and dCas9 constructs were delivered by lentivirus, partially selected with puromycin and blasticidin, and then stained for target gene expression. Safe-targeting sgRNA and dCas9 are negative controls. The shaded histogram shows the cells after gating for both the dCas9 (BFP) and the sgRNA (mCherry) and their percentage of cells OFF is shown, while the unshaded histogram shows the cells from the same sample that express neither and serve as an internal control. FIG. 47C is dCas9 recruitment to the endogenous surface marker gene CD81 in K562 cells. First, dCas9 constructs were delivered by lentivirus, then sgRNAs were delivered by lentivirus, and 3 days later the cells were selected with puromycin and blasticidin. Then, 9 days after sgRNA infection, the cells were stained for CD81 expression, fixed, and analyzed by flow cytometry. The shaded histogram shows the cells expressing both the dCas9 vector (BFP) and the sgRNA (mCherry) their percentage of cells OFF is shown, while the unshaded histogram shows the cells from the same sample that express neither. FIG. 47D is Cas9 recruitment of KRAB paralogs to the endogenous gene CD43 in K562 cells. dCas9-repressors were delivered by lentivirus to cells that stably express sg10 or sg15, three days later blasticidin selection was initiated, and then cells were stained for CD43 expression 9 days after dCas9 infection. The shaded histogram shows the cells expressing both the dCas9 vector (BFP) and the sgRNA (mCherry) and their percentage of cells OFF is shown, while the gray unshaded histogram shows the cells from the same sample that express neither and serve as an internal control. FIG. 47E is a schematic of a CRISPRi benchmarking screen for comparing KRAB repressors. A library of guides targeting essential genes and enhancers was delivered into K562 cell lines that stably express a dCas9-repressor fusion, cells were passaged for 14 days, then the guides were sequenced to measure fitness effects. FIG. 47F is results from CRISPRi benchmarking screen targeting the promoters of essential genes. Greater depletion over 14 days of growth relative to the original plasmid pool representation is associated with stronger effector-mediated silencing of the essential genes. Violin shows distribution of average sgRNA-level depletion from two screen replicates, solid line shows median, dotted lines are quartiles (N=405 sgRNAs, **** denotes P<0.0001 by Kruskal-Wallis test). The dashed line at 0 represents the median of the safe-targeting negative controls. FIG. 47G is a comparison of baseline silencing with ZNF10 KRAB and relative improvement with ZNF705F KRAB from CRISPRi benchmarking screen targeting the promoters of 37 essential genes. Effect sizes are the log₂(fold-change) of sgRNA representation after 14 days of growth relative to the original plasmid pool representation. The median effect across 8-10 sgRNAs per gene was computed and each dot shows its average for two infection replicate screens. Horizontal and vertical error bars show the ranges. Dashed line shows parity between KRAB domains. FIG. 47H, top, is a schematic of dCas9-repressor recruitment at the pEF1a-TagRFP-T reporter in HEK293T using TetO targeting sgRNA. FIG. 47H, bottom, is transient expression and targeting of dCas9-KRAB paralogs fusions to the TetO sites upstream of the reporter gene. Means are from two biological replicates. Percentage of cells OFF were normalized to safe-targeting sgRNA. FIG. 47I shows that after targeting the dCas9-KRAB paralog fusions for 5 days by transient transfection at the TagRFP reporter in HEK293T cells, silenced cells were sorted, and memory dynamics was measured by flow cytometry throughout 35 days. Each dot is a biological replicate (n=2). The percentage of cells OFF were normalized to safe-targeting sgRNA. FIG. 47J is a comparison of baseline silencing with ZNF10 KRAB and relative improvement with ZNF705F KRAB when dCas12a fusions were recruited to silence CD43 or CD32. Each dot is colored by the target gene and shows the average for two infection replicates of a guide RNA. Horizontal and vertical error bars show the ranges. Dashed line shows parity between KRAB domains.

FIGS. 48A-48C show HT-recruit systematically quantifies transcriptional effector functions across DNA-binding domain, cell-type, and target gene contexts. FIG. 48A is a schematic of high-throughput recruitment (HT-recruit) to quantify transcriptional effector function at scale while varying the context of DNA-binding domains (DBDs), cell type, and target reporters or endogenous genes. A pooled library of all Pfam domains≤80 amino acids from human nuclear proteins is synthesized as 300-mer oligonucleotides, cloned downstream of the doxycycline (dox)-inducible rTetR DNA-binding domain (DBD) or dCas9 (Context 1), and delivered to either K562 or HEK293T cells (Context 2) at a low multiplicity of infection (MOI) such that the majority of cells express a single DBD-domain fusion. The target gene (inset) can be silenced or activated by recruitment of repressor or activator domains to the promoter. The synthetic reporters can be driven by different promoters (Context 3) and encode a synthetic surface marker (Igκ-hIgG1-Fc-PDGFRβ, purple) and fluorescent marker (Citrine, yellow), separated by a T2A self-cleaving peptide (grey). These reporters are stably integrated into the AAVS1 safe harbor locus using TALEN-mediated homology directed repair. The endogenous target genes encode for surface markers (Context 3). After recruitment of Pfam domains, ON and OFF cells were magnetically separated using beads that bind these synthetic or endogenous surface markers, and the domains were sequenced in the Bound and Unbound populations to compute enrichments. FIG. 48B shows representative expression levels of varied synthetic reporters in K562 and HEK293T measured by flow cytometry. Each Citrine reporter uses a different promoter: minCMV, nonTATAchr21 (nTchr21), nonTATAchrX (nTchrX), UbC, PGK, or pEF1a. The minimal reporter promoters, expected to be activatable, were minCMV, nTchrX, and nTchr21 and the longer promoters, expected to be repressible, were pEF1a, PGK, UbC, and RSV. The promoters can be silenced or activated by doxycycline-mediated recruitment of repressor or activator domains via rTetR at the 9× TetO site upstream of the promoter. Positive control effectors, ZNF10 KRAB repressor or VP64 activator, were delivered by lentivirus in both cell types. Cells were treated with 1000 ng/mL doxycycline for 5 days for repression and 2 days for activation and Citrine expression was measured by flow cytometry after gating for mCherry delivery. Silencing of the reporter by KRAB is shown in blue and activation by VP64 in red. Basal no recruitment/dox level shown in grey. FIG. 48C shows representative expression levels of endogenous surface marker genes CD2 and CD43 in K562 as measured by immunostaining and flow cytometry. Positive control effectors, dCas9-KRAB (blue) or dCas9-VP64 (red), and negative control, dCas9 only or sgRNA only (black), were delivered by lentivirus and selected by blasticidin (dCas9) and puromycin (sgRNA).

FIGS. 49A-49C show HT-recruit to various reporters in K562 and HEK293T cells FIG. 49A shows the observation of background silencing at the RSV, PGK, and UbC promoters in K562 and HEK293T cells. In K562 cells, the RSV reporter was significantly silenced over time. While in HEK293T, the RSV, PGK, and UbC promoters were slightly background silenced. Purple line represents cells at a later time point (at least 2 weeks later). FIG. 49B shows the correlation of mean fluorescent intensity (MFI) of the Citrine fluorescent reporter under different promoters: minCMV, nTchr21, nTchrX, PGK, UbC, pEF1a, PGK, and RSV. Reporters were stably integrated at the AAVS1 safe harbor locus in K562 and HEK293T cells by TALENs. Each dot represents a mean taken from three replicates. A simple linear regression was performed to determine the relationship between promoters in these two cell types (R²=0.86). FIG. 49C shows the results from testing various CD43 sgRNAs (sg10-15) in K562 cells to identify guides that allow for analysis of KRAB-mediated silencing of the CD43 gene. Silencing was measured by CD43 (Alexa Fluor 647) surface marker immunostaining and flow cytometry 7 days after lentiviral sgRNA infection in stable dCas9-KRAB (blue) or dCas9 (black) cell lines.

FIGS. 50A-50H show the effect of cell type and promoter on transcriptional effectors. FIG. 50A shows individual validations of repressor hits identified in FIG. 34 across K562 and HEK293T cells. rTetR-repressor fusions or the rTetR-only negative control were delivered via lentivirus to the pEF1a reporter cells. After selection, cells were treated with 1000 ng/ml doxycycline for 5 days to induce reporter repression. The percent of cells silenced was measured by flow cytometry for the Citrine reporter after gating for delivery with mCherry. Mean percentage of cells silent were calculated from 2 independently transduced replicates for K562 and 2 biological replicates for HEK293T; error bars are standard deviation. FIG. 50B shows individual validations of activator hits identified in FIG. 29, top left, across K562 and HEK293T as measured by average percentage of cells ON normalized to no dox control. rTetR-activators were delivered, selected, and quantified by flow cytometry as described in FIG. 50A. Cells were treated with 1000 ng/ml doxycycline for 2 days to induce reporter activation (n=2 independently transduced replicates for K562 and n=3 for HEK293T). Cell type specific activators are shown as pink dots based on a 20 hit threshold from the minCMV activator screen. FIG. 50C shows HT-recruit with dCas9 targeting endogenous gene CD43 compared with rTetR targeting the pEF1a reporter in K562. Repression strength is measured by log₂(OFF:ON) (n=2 biological replicates per cell type). FIG. 50D shows individual validation of repressors across contexts in K562 cells. DBD-repressor fusions or the DBD-only negative control were delivered to (left) pEF1a reporter cells and cells were treated with doxycycline for 6 days and measured 10 days after dCas9-repressor infection in CD43 sgRNA-expressing cell lines. The percentage of cells silenced was measured by flow cytometry for the citrine reporter after gating for delivery with mCherry (n=2 independently transduced replicates) or flow cytometry for CD43 staining after gating for delivery with BFP (n=1 per sgRNA). FIG. 50E shows HT-recruit with dCas9 to activate the endogenous gene CD2 using guide sg717 compared with activation of minCMV promoter with rTetR in K562 cells. Activation strength is measured by log₂(OFF:ON) (n=2 biological replicates per cell type). FIG. 50F is a scatter plot comparing HLH activation of CD2 with dCas9 but repression of pEF promoter with rTetR. Stronger HLH activators of CD2 are also stronger repressors of pEF. HT-recruit with dCas9 to activate the endogenous gene CD2 using guide sg717 compared with repression of pEF1a promoter with rTetR in K562 cells. HLH domains (yellow dots) were identified to activate the CD2 gene while strongly repressing the pEF1a reporter. FIG. 50G shows recruitment of HLH domains from NeuroG2, HAND2, ID2, and TWIST1 to the pEF1a reporter in K562 cells. Shown are Citrine fluorescence distributions after 6 days of recruitment (dox treatment) in cells stably expressing the rTetR-HLH fusions, rTetR-KRAB positive control, or rTetR-only negative control. The percentage of cells with the reporter silenced is shown (left of black line). FIG. 50H shows individual validation of dCas9-HLH (domain from NeuroG2) activation at the CD2 endogenous gene in K562 cells. Two sgRNAs (sg717 and sg718) were used to target the effector domain to the CD2 gene. dCas9 only and dCas9-VP64 are negative and positive controls, respectively. The percentage of cells activated is shown.

FIG. 51 shows individual validations of repressor domains in HEK293T cells as seen in FIG. 50A. rTetR(SE-G72P)-domain fusions were delivered to pEF1a reporter cells by lentivirus and selected with blasticidin. Cells were treated with 1,000 ng/mL dox for 4 days in HEK293T and 5 days in K562, and then Citrine reporter levels were measured by flow cytometry. Untreated cell distributions are shown in light gray and dox-treated cells are shown in colors, with 2 biological replicates in each condition. The dotted vertical line shows the Citrine gate used to determine the fraction of cells OFF. The average percentage of cells OFF normalized to no dox control is shown. ZNF10 KRAB and rTetR only were included as positive and negative controls, respectively.

FIGS. 52A-52H show activators across K562 and HEK293T cells. FIG. 52A shows individual validations of strong activator domains (FOXO-TAD, LMSTEN, NCOA3, ZNF473 KRAB) at the minCMV reporter in HEK293T cells. rTetR(SE-G72P)-domain fusions were delivered to minCMV reporter cells by lentivirus and selected with blasticidin. Cells were treated with 1,000 ng/mL dox for 2 days. Untreated cell distributions are shown in light gray and dox-treated cells are shown in colors, with 2 biological replicates in each condition. The dotted vertical line shows the Citrine gate used to determine the fraction of cells ON. The average percentage of cells ON normalized to no dox control is shown. VP64 and rTetR only were included as positive and negative controls, respectively. FIG. 52B shows individual validations of cell-type specific activator domains in HEK293T (top) and K562 (bottom) cells as seen in FIG. 50D. rTetR(SE-G72P)-domain fusions were delivered to minCMV reporter cells by lentivirus and selected with blasticidin. Cells were treated with 1,000 ng/ml dox for 2 days, and then citrine reporter levels were measured by flow cytometry. Untreated cell distributions are shown in light gray and dox-treated cells are shown in colors, with 3 replicates in each condition. The dotted vertical line shows the citrine gate used to determine the fraction of cells ON, and the average fraction ON for the dox-treated cells is shown. FIG. 52C shows FLAG-staining of Pfam domains to compare well-expressed domains between HEK293T and K562 cells (Spearman rho=0.84). Well-expressed domains were identified based on a 1σ hit threshold above the random controls. FIG. 52D, left, shows the percentage of CD2 endogenous gene activated after transient expression and targeting of dCas9-effector fusions to the CD2 endogenous gene for 3 days in K562 cells using sg717 and sg718 guides. Cells were immunostained for CD2 (APC) expression and analyzed by flow cytometry after gating for dCas9 (TagBFP) and sgRNA (GFP). Each dot represents an independently transfected sgRNA (n=2-4). FIG. 52D, right, shows the percentage of CD2 endogenous gene activated after transient expression and targeting of dCas9-domain fusions to the gene using multiple sgRNAs (sg39, sg42, sg717) for 3 days in HEK293Ts. Cells were immunostained for CD2 (APC) expression and analyzed by flow cytometry after gating for dCas9 (TagBFP) and sgRNA (GFP). Each dot represents an independently transfected biological replicate (n=2). dCas9 only and VPR are negative and positive controls, respectively. FIG. 52E is a graph comparing the percentage of K562 and HEK293T cells with Citrine silenced after 2 days of SMARCA4 QLQ (tiles around the QLQ domain) recruitment with doxycycline. Means are from 2 replicates. FIG. 52F is a graph comparing the percentage of K562 and HEK293T cells with Citrine silenced after 2 days of SMARCA2 QLQ recruitment with doxycycline and BRM014, a SMARCA2/4 ATPase inhibitor. Means are from 2 replicates; statistical analysis by two-tailed unpaired t-test (K562: **p=0.0025; HEK293T: ***p=0.0001). FIG. 52G is a graph comparing the percentage of HEK293T cells with Citrine silenced after 2 days of VP64 and FOXO3 FOXO-TAD recruitment with doxycycline and BRM014. Means are from 2 replicates; statistical analysis by two-tailed unpaired t-test (VP64: **p=0.0069; FOXO: ***p=0.0004). FIG. 52H is a graph comparing the percentage of HEK293T cells with Citrine activated after 2 days of QLQ recruitment with doxycycline and SMARCA2/4 knockdown with siRNA. Means are from 4 replicates; statistical analysis by two-tailed Tukey's test (Ctrl vs. SMARCA2: *p=0.040; SMARCA2 vs. SMARCA4: ****p=1.3e-9; SMARCA4 vs. SMARCA2/4: ****p=3.2e-11).

FIGS. 53A-53H show repressors across targets. FIG. 53A shows the comparison of HT-recruit screen at the endogenous CD2 gene versus the pEF1a reporter in K562 cells. FIG. 53B shows dCas9 recruitment of Pfam domains with an sgRNA that binds the TetO site upstream the pEF1a reporter in K562 cells. FIG. 53C shows individual rTetR-mediated recruitment of Pfam domains at the PGK reporter in K562 cells. FIG. 53D shows individual rTetR-mediated recruitment of Pfam domains at the pEF reporter in K562 cells. FIG. 53E shows individual dCas9-mediated recruitment of Pfam domains at the endogenous CD43 gene in K562 cells. Cell lines stably expressing the sgRNA were infected with dCas9-repressors lentiviruses, then fixed and stained for analysis by flow cytometry 10 days after infection. Shaded histograms show cells gated for the sgRNA (mCherry) and dCas9 delivery (BFP), whereas the light gray histogram shows cells that express neither and serve as an internal control. FIG. 53F shows activating effector domains (FOXO-TAD, QLQ, LMSTEN, NCOA3) fused to rTetR were stably integrated into K562 and HEK293T cells. Domains were recruited to the constitutive pEF1a reporter in both cell types for 5 days with dox and then analyzed by flow cytometry for repression. Untreated cell distributions are shown in light gray and dox-treated cells are shown in colors, with 3 biological replicates in each condition. The two dotted vertical line shows the Citrine gate used to determine the fraction of cells OFF and ON, respectively. ZNF10 KRAB and rTetR only were included as positive and negative controls, respectively. FIG. 53G shows the percentage of K562 or HEK293T cells OFF after recruitment with activating effector domains in FIG. 53F The average percentage of cells OFF normalized to no dox control is shown (n=3 biological replicates). FIG. 53H shows the percentage of K562 or HEK293T cells super-activated to a higher ON state in FIG. 53F. The average percentage of cells ON normalized to no dox control is shown (n=3 biological replicates).

FIGS. 54A-54G show activators across targets. FIG. 54A, top, shows validation of SMARCA2 QLQ and CXXC1 and PYGO1 PHD activator domains across core promoter reporters (minCMV, nTchr21, nTchrX) in K562 cells. rTetR-activator fusions or the rTetR-only negative control were delivered by lentivirus to reporter cells. After selection, cells were treated with 1000 ng/ml doxycycline for 2 days to induce reporter activation. The percent of cells activated was measured by flow cytometry for the Citrine reporter, after gating for delivery with mCherry (n=3 replicates). FIG. 54A, bottom is a bar plot quantifying recruitment of effector domain at the promoters in K562 cells. FIG. 54B shows individual validations of activators hits across PGK and minCMV promoter types in HEK293T as measured by average percentage of cells ON normalized to no dox control. Cells were treated with 1000 ng/ml doxycycline for 2 days to induce reporter activation (n=2 independently transduced replicates for each promoter type). FIG. 54C shows locations of the CD2 targeting guides (sg39 sg89, sg717, sg718, sg42) at the CD2 promoter region. FIG. 54D shows dCas9 recruitment of activators with an sgRNA that binds the TetO site upstream the minCMV reporter in K562 cells. FIG. 54E shows the comparison of HT-recruit when dCas9 is targeted to the CD2 gene using two different guides (sg717 and sg718) in K562 cells. FIG. 54F shows recruitment of dCas9-activator fusion hits at the CD2 gene using two different guides (sg717 and sg718) in K562 cells. sgRNA were stably delivered by lentivirus and selected for with puromycin, then dCas9 fusion plasmids were delivered by electroporation, then cells were analyzed 3 days later by flow cytometry for surface stained CD2 after gating for dCas9 (BFP) and sgRNA (GFP). The percentage of cells ON is shown. N+F+Z is a combination of three activators, described in FIG. 42, that is used as a positive control. The 80 AA sequences match the library elements while the trimmed sequences match the annotated Pfam domain. The polyQ is a homopolymer of 15 repeated glutamines, which is also found at the C-terminus of the 80 AA QLQ and is not present in the trimmed QLQ. FIG. 54G shows dCas9-HLH activate CD2 but not CD20 or CD28. dCas9 fusions were delivered to K562 cells by lentivirus and selected for with blasticidin, then sgRNAs were delivered by lentivirus and selected with puromycin, then 8 days after sgRNA delivery the cells were stained for the targeted surface marker genes and measured by flow cytometry. Surface marker expression is shown after gating for dCas9 with BFP and sgRNA with GFP.

FIGS. 55A-55I show synergistic repression with a RYBP-MGA combination. FIG. 55A shows recruitment of dCas9-effectors to the endogenous surface marker gene, CD43, in K562 cells. Additional results from the experiment described in FIG. 47B are shown. Means and standard deviations are from two independent infections using either CD43 sg10 or sg15, after gating for dCas9 (BFP) and guide (mCherry) expression. Percentage of cells OFF were normalized to safe-targeting sgRNA N4293. FIG. 55B shows transient expression and recruitment of dCas9-Pfam domain fusions to the TetO sites upstream of the reporter gene in HEK293T cells for 5 days. Means and standard deviations are from two biological replicates after gating for dCas9 (BFP) and guide (mIFP) expression. Percentage of cells OFF were normalized to safe-targeting sgRNA. FIG. 55C shows recruitment of dCas9-repressors and combinations to the endogenous CD43 gene in K562 cells. Additional results from the experiment described in FIG. 47D are shown, wherein silencing is measured 9 days after dCas9 infection. Means and standard deviations are from two independent infections using either CD43 sg10 or sg15, after gating for dCas9 (BFP) and guide (mCherry) expression. The RYBP domain from the RYBP protein (RYBP_RYBP) was used in the repressor combinations. FIG. 55D shows recruitment of repressors combinations to CD81 in K562 cells using sg3, after gating for dCas9 (BFP) and guide (mCherry) expression. First the dCas9 fusions were stably delivered by lentivirus and selected for with blasticidin, then the sgRNA was delivered by lentivirus and 9 days later the cells were stained and fixed for flow cytometry analysis. The percentage of cells OFF is shown. FIG. 55E shows transient expression and recruitment of various dCas9-repressor combo fusions from FIG. 55A to the TetO sites upstream of the reporter gene in HEK293T cells for 5 days. Means are from two biological replicates after gating for dCas9 (BFP) and guide (mIFP) expression. Percentage of cells OFF were normalized to safe-targeting sgRNA. FIG. 55F shows chromatin modifications mapped by CUT&RUN after dCas9 recruitment of repressors to the CD43 endogenous gene using sg15. Stable lines expressing both the dCas9 fusion and the sgRNA were selected with both antibiotics and FACS before chromatin was analyzed. In FIG. 55G, after targeting the dCas9-repressor combo fusions for 5 days by transient transfection at the TagRFP reporter in HEK293T cells, silenced cells were sorted, and memory dynamics was measured by flow cytometry throughout 30 days. Each dot is a biological replicate (n=2). The percentage of cells OFF at each day was normalized to safe-targeting sgRNA. FIG. 55H shows endo Repr screen targeting growth genes with repressors and combinations. FIG. 55I shows individual dCas9-mediated recruitment of repressor combinations at the endogenous CD43 gene in K562 cells. Cell lines stably expressing the sgRNA were infected with dCas9-repressors lentiviruses, then fixed and stained for analysis by flow cytometry 10 days after infection. Shaded histograms show cells gated for the sgRNA (mCherry) and dCas9 delivery (BFP); the darker shade is sg10 and the lighter colored shade is sg15, and the light gray histogram shows cells that express neither and serve as an internal control. Control lines are shared with FIG. 53E, which was performed in parallel. The average percentage of cells OFF is shown.

FIG. 56 shows dCas12a fusions to NFZ and QNZF to activate endogenous CD2. NFZ and QNZF, or VP64 as a control, were fused to dCas12a. 1 μg of dCas12a plasmid and 1 ug of a CD2-targeting or safe-control gRNA expression plasmid with an mCherry marker was delivered to K562 cells by co-electroporation. After 3 days, cells were stained for CD2 with APC-conjugated antibody and activation was measured by flow cytometry. mCherry-APC bleedthrough compensation was applied and data was gated for high gRNA delivery (mCherry). Two replicates are shown as shaded histograms, and the mean percentage of CD2-targeted and activated cells is shown.

### DETAILED DESCRIPTION

Systems and methods to generate a catalog of compact transcriptional effector domains is provided. Further, in some embodiments, this catalog of domains is fused onto DNA binding domains to engineer synthetic transcription factors. These find use to perform targeted and tunable regulation of gene expression in eukaryotic (or other) cells. This technology leverages a high-throughput platform to screen and characterize tens of thousands of synthetic transcription factors in cells. These synthetic transcription factors are fusions between a DNA binding domain and a transcriptional effector domain. The system has been used to generate hundreds of short effector domains (e.g., 80 amino acids) and a high-throughput process for shortening them further to the minimally sufficient sequences (e.g., 10 amino acids), which is an advantage for delivery (e.g., packaging in viral vectors). The targeting of these fusions generates local regulation of mRNA transcription, either negatively or positively depending on the effector domain. Some of these synthetic transcription factors mediate long-term epigenetic regulation that persists after the factor itself has been released from the target.

Previously, a limited number of transcriptional effector domains were available for the engineering of synthetic transcription factors. To address this limitation, provided herein is a high-throughput approach to screening and quantifying the function of transcriptional effectors domains. This approach enabled the discovery of hundreds of effector domains that can upregulate or downregulate transcription in a targeted manner when fused onto a DNA binding domain. This process also finds use to identify mutants of effector domains with enhanced activity. These effector domains find use to engineer synthetic transcription factors for applications in gene and cell therapy, synthetic biology, and functional genomics.

Exemplary applications include, but are not limited to:

Targeted repression/activation of endogenous genes with fusions of programmable DNA binding domains (e.g., dCas9, dCas12a, zinc finger, TALE) to transcriptional effector domains.

Targeted repression/activation of exogenous genes with fusions of programmable DNA binding domains (e.g., dCas9, dCas12a, zinc finger, TALE) to transcriptional effector domains.

Gene and cell therapy (e.g., to silence a pathogenic transcript in a patient) or in research.

Synthetic transcription factors find use to perturb the expression of multiple genes simultaneously (e.g., to perform high-throughput genetic interaction mapping with CRISPRi/a screens using multiple guide RNAs).

Use in synthetic transcription factors in genetic circuits, e.g., inducible gene expression or more complex circuits. These circuits find use in gene therapy (e.g., AAV delivery of antibodies) and cell therapy (e.g., ex vivo engineering of CAR-T cells) to achieve therapeutic gene expression outputs in response to environmental and small molecule inputs.

The new transcriptional effector domains provided herein have several advantages for applications that rely on synthetic transcription factors. Short domains were identified (e.g., 80 amino acids or less) and a high-throughput process was generated for shortening them further to the minimally sufficient sequence, which is an advantage for delivery (e.g., packaging in viral vectors). In some cases, potent effector domains were identified that were as short as 10 amino acids. In some embodiments, the domains are extracted from human proteins, which provides the advantage of reducing immunogenicity in comparison to viral effector domains. Most of the domains generated have not been reported as transcriptional effectors previously. In addition, a high-throughput process is provided for testing mutations in these domains in order to identify enhanced variants. The high-throughput approach is more readily aided by the development of an artificial cell surface marker that provides more efficient, inexpensive, and rapid screening of these libraries using magnetic separation. This is an advantage over the more conventional approach of sorting libraries based on fluorescent reporter gene expression.

The collection of domains identified is large and diverse, and the platform readily enables new combinations of domains to be tested as fusions in high-throughput to create synthetic transcription factors with new properties (e.g., compositions of two repressor domains to achieve a combination of fast silencing and permanent silencing).

Hundreds of previously uncharacterized or unknown effector domains that can silence or active transcription and can be fused onto DNA binding domains. For example, a high-throughput approach for screening single domains and pairs of domains using lentiviral screens in human cells is provided. The high-throughput approach is more readily enabled by the development of an artificial cell surface marker that provides more efficient, inexpensive, and rapid screening of these libraries using a magnetic separation.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “antibody,” as used herein, refers to a protein that is endogenously used by the immune system to identify and neutralize foreign objects, such as bacteria and viruses. Typically, an antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other. The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)). The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the invention. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H1} domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics when compared to the wild-type gene or gene product.

### 2. Method for Identifying Transcriptional Modifying Domains

Disclosed herein are methods for identifying transcriptional effector (e.g., activator and repressor) domains. In some embodiments, the methods comprise: preparing a domain library comprising a plurality of nucleic acid sequences each configured to express a fusion protein comprising a protein domain from nuclear-localized proteins linked to an inducible DNA binding domain; transforming reporter cells with the domain library, wherein the reporter cells comprises a two-part reporter gene comprising a surface marker and a fluorescent protein under the control of a promoter, wherein the two-part reporter gene is capable of being modulated by a putative transcriptional effector domain following treatment with an agent configured to induce the inducible DNA binding domain; treating the reporter cells with the agent for a length of time necessary for protein and mRNA levels to be altered in the cell (e.g., increased due to production or decreased due to degradation); sequencing the protein domains from the separated reporter cells; calculating for each protein domain sequence a ratio of sequencing counts from reporter cells not having the surface marker, the fluorescent protein, or a combination thereof to sequencing counts from reporter cells having the surface marker, the fluorescent protein, or a combination thereof, and identifying protein domains as transcriptional repressors or activators.

The methods comprise preparing a domain library comprising a plurality of nucleic acid sequences each configured to express a fusion protein comprising a protein domain from nuclear-localized proteins linked to an inducible DNA binding domain. The protein domain may be less than or equal to 80 amino acids. In some embodiments, the protein domain may be about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, or about 5 amino acids.

The protein domain may be derived from any known protein. In some embodiments, the protein domain is from a nuclear-localized protein. A nuclear-localized protein includes those proteins which are or can localize to the nucleus fully or partially during the life-cycle of the protein. In some embodiments, the protein domain comprises amino acid sequences of the wild-type protein domain from nuclear-localized proteins. In some embodiments, the protein domain comprises mutated amino acid sequences of protein domains from nuclear-localized proteins.

The inducible DNA binding domain may use any system for induction of DNA binding, including, but not limited to, tetracycline Tet/DOX inducible systems, light inducible systems, Abscisic acid (ABA) inducible systems, cumate systems, 40HT/estrogen inducible systems, ecdysone-based inducible systems, and FKBP12/FRAP (FKBP12-rapamycin complex) inducible systems.

In some embodiments, the inducible DNA binding domain comprises a tag. The tag may include any tag known in the art, including tags removable by chemical or enzymatic means. Suitable tags for use in the present method include chitin binding protein (CBP), maltose binding protein (MBP), Strep-tag, glutathione-S-transferase (GST), a polyhistidine (PolyHis) tag, an ALFA-tag, a V5-tag, a Myc-tag, a hemagglutinin (HA)-tag, a Spot-tag, a T7-tag, an NE-tag, a Calmodulin-tag, a polyglutamate tag, a polyarginine tag, a FLAG tag, and the like.

The methods comprise transforming reporter cells with the domain library, wherein the reporter cell comprises a two-part reporter gene comprising a surface marker and a fluorescent protein under the control of a promoter, wherein the two-part reporter gene is capable of being modulated by a putative transcriptional effector domain following treatment with an agent configured to induce the inducible DNA binding domain.

The promoter may confer a high rate of transcription (a strong promoter) or confer a low rate of transcription (weak promoter). Many promoter libraries have been established experimentally and choice of promoter and promoter strength is dependent on cell type. In some embodiments, when identifying transcriptional activator domains, a weak promoter may be used. In some embodiments, when identifying transcriptional repressor domains, a strong promoter may be used.

Cell surface markers include proteins and carbohydrates which are attached to the cellular membrane. Cell surface markers are generally known in the art for a variety of cell types and can be expressed in a reporter cell of choice based on known molecular biology methods. The surface marker may be a synthetic surface marker comprising marker polypeptide attached to a transmembrane domain. For example, the marker polypeptide may include an antibody or a fragment thereof (e.g., Fc region) attached to a transmembrane domain. In some embodiments, the marker polypeptide is human IgG1 Fc region and the synthetic surface marker comprises human IgG1 Fc region attached to a transmembrane domain.

Fluorescent proteins are well known in the art and include proteins adapted to fluoresce in various cellular compartments and as a result of varying wavelengths of incoming light. Examples of fluorescent proteins include phycobiliproteins, cyan fluorescent protein (CFP), green fluorescent protein (GFP), yellow fluorescent protein (YFP), enhanced orange fluorescent protein (OFP), enhanced green fluorescent protein (eGFP), modified green fluorescent protein (emGFP), enhanced yellow fluorescent protein (eYFP) and/or monomeric red fluorescent protein (mRFP) and derivatives and variants thereof.

The methods comprise separating reporter cells based on presence or absence of the surface marker, the fluorescent protein, or a combination thereof. A number of cell separation techniques are known in the art are suitable for use with the methods disclosed herein, including, for example, immunomagnetic cell separation, fluorescent-activated cell sorting (FACS), and microfluidic cell sorting. In some embodiments, cell separation comprises immunomagnetic cell separation.

In some embodiments, the method further comprises stopping treatment of the reporter cells with the agent and repeating the separating, sequencing, calculating, and identifying steps one or more times. In some embodiments, the steps are repeated at least 48 hours after stopping treatment of the reported cells with the agent.

In some embodiments, the method further comprises measuring expression level of protein domains. The expression level of the protein domains can be determined using any methods known in the art, including immunoblotting and immunoassays for the protein itself or any tags or labels thereof. In some embodiments, the expression level is determined by measuring a relative presence or absence of the tag on the DNA binding domain.

In some embodiments, the methods identify a transcriptional repressor domain. In some embodiments, the methods comprise, a) preparing a domain library comprising a plurality of nucleic acid sequences each configured to express a fusion protein comprising a protein domain linked to an inducible DNA binding domain; b) transforming reporter cells with the domain library, wherein a reporter cell comprises a two-part reporter gene comprising a surface marker and a fluorescent protein under the control of a strong promoter, wherein the two-part reporter gene is capable of being silenced by a putative transcriptional repressor domain following treatment with an agent configured to induce the inducible DNA binding domain; c) treating the reporter cells with the agent for a length of time necessary for protein and mRNA degradation in the cell; d) separating reporter cells based on presence or absence of the surface marker, the fluorescent protein, or a combination thereof; e) sequencing the protein domains from the separated reporter cells; f) calculating for each protein domain sequence a ratio of sequencing counts from reporter cells not having the surface marker, the fluorescent protein, or a combination thereof to sequencing counts from reporter cells having the surface marker, the fluorescent protein, or a combination thereof; and g) identifying protein domains as transcriptional repressor.

In some embodiments, the reporter cells are treated with the agent for at least 3 days. For, example the reporter cells may be treated with the agent for at least 3 days, at least 4 days, at least 5 days, at least 6 days, at least 7 days, at least 8 days, at least 9 days, at least 10 days, at least 14 days, or more. In some embodiments, the reporter cells at treated with the agent for 3-12 days, 3-10 days, 3-7 days, or 3-5 days.

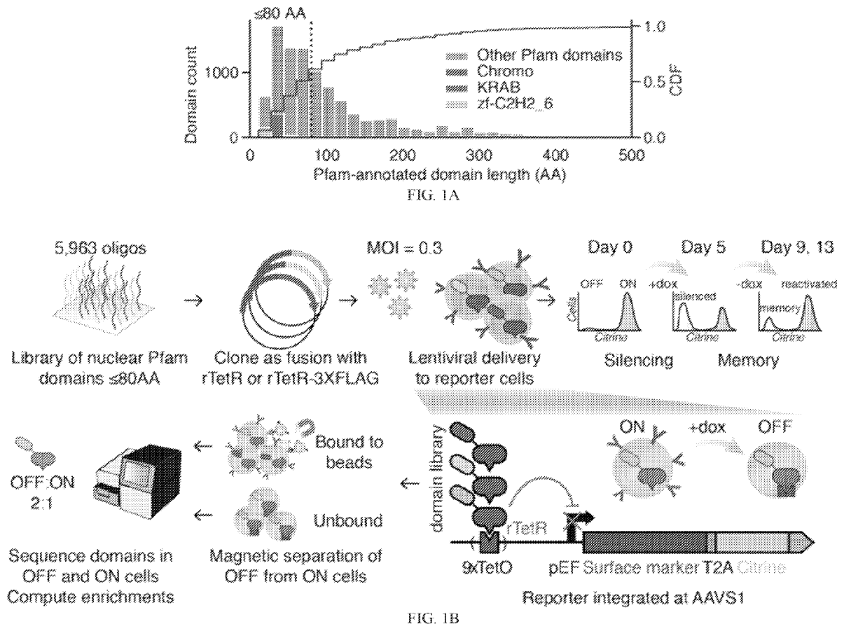

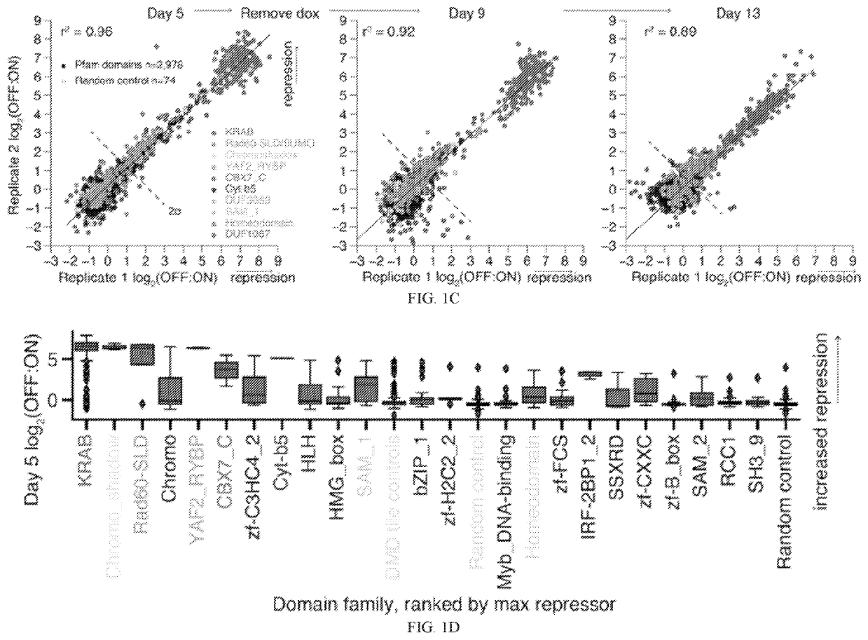

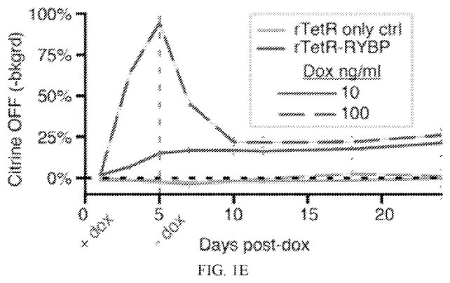

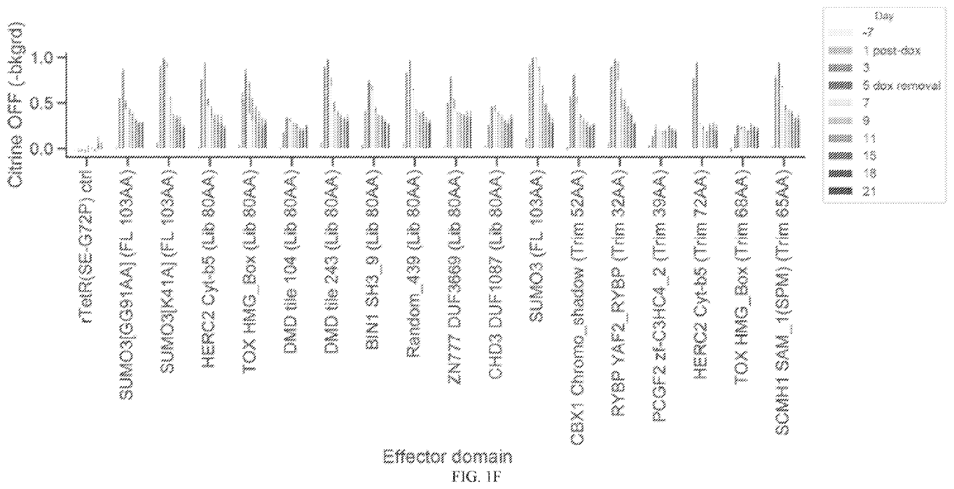

The protein domain is identified as a transcriptional repressor when log 2 of the ratio of sequencing counts from reporter cells not having the surface marker, the fluorescent protein, or a combination thereof to sequencing counts from reporter cells having the surface marker, the fluorescent protein, or a combination thereof is at least two standard deviations from (e.g., greater than) the mean of a negative control (See FIG. 1C, for example).

In some embodiments, the methods identify a transcriptional activator domain. In some embodiments, the methods comprise, a) preparing a domain library comprising a plurality of nucleic acid sequences each configured to express a fusion protein comprising a protein domain linked to an inducible DNA binding domain; b) transforming reporter cells with the domain library, wherein the reporter cells comprises a two-part reporter gene comprising a surface marker and a fluorescent protein under the control of a weak promoter, wherein the two-part reporter gene is capable of being activated by a putative transcriptional activator domain following treatment with an agent configured to induce the inducible DNA binding domain; c) treating the reporter cells with the agent for a length of time necessary for protein and mRNA production in the cell; d) separating reporter cells based on presence or absence of the surface marker, the fluorescent protein, or a combination thereof; e) sequencing the protein domains from the separated reporter cells; f) calculating for each protein domain sequence a ratio of sequencing counts from reporter cells not having the surface marker, the fluorescent protein, or a combination thereof to sequencing counts from reporter cells having the surface marker, the fluorescent protein, or a combination thereof; and g) identifying protein domains as transcriptional repressor.

In some embodiments, the reporter cells are treated with the agent for at least 24 hours. For, example the reporter cells may be treated with the agent for at least 24 hours (1 day), at least 36 hours, at least 48 hours (2 days), at least 60 hours, at least 72 hours (3 days), at least 94 hours, at least 106 hours (4 days) or more. In some embodiments, the reporter cells are treated for between 24 and 72 hours or between 36 and 60 hours.

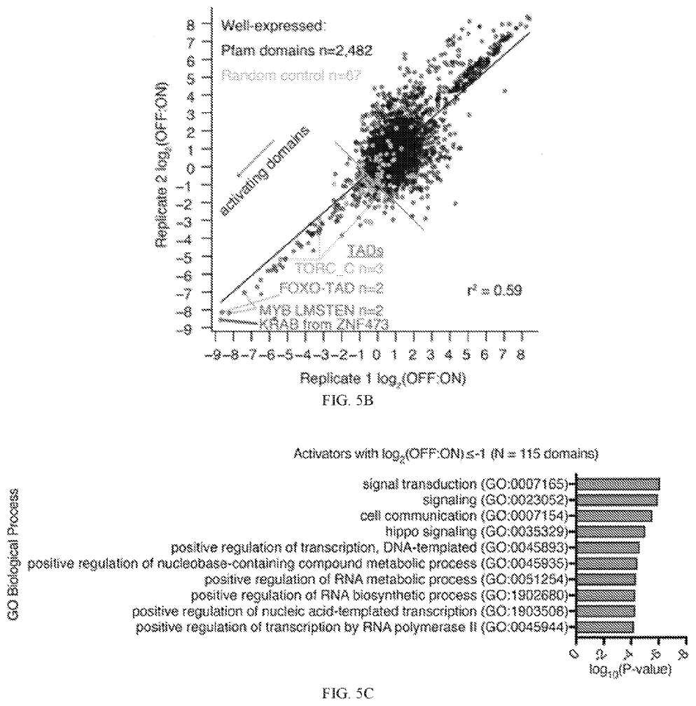

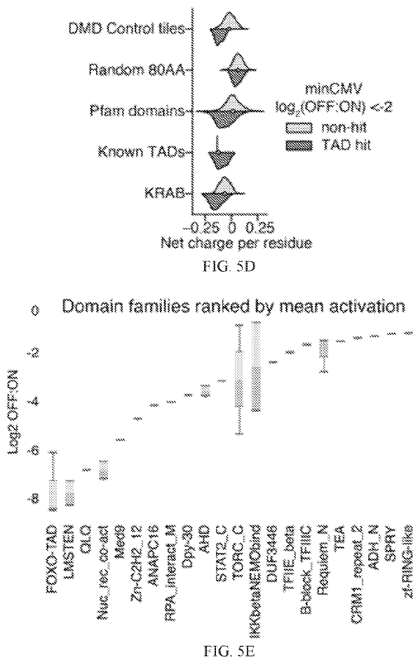

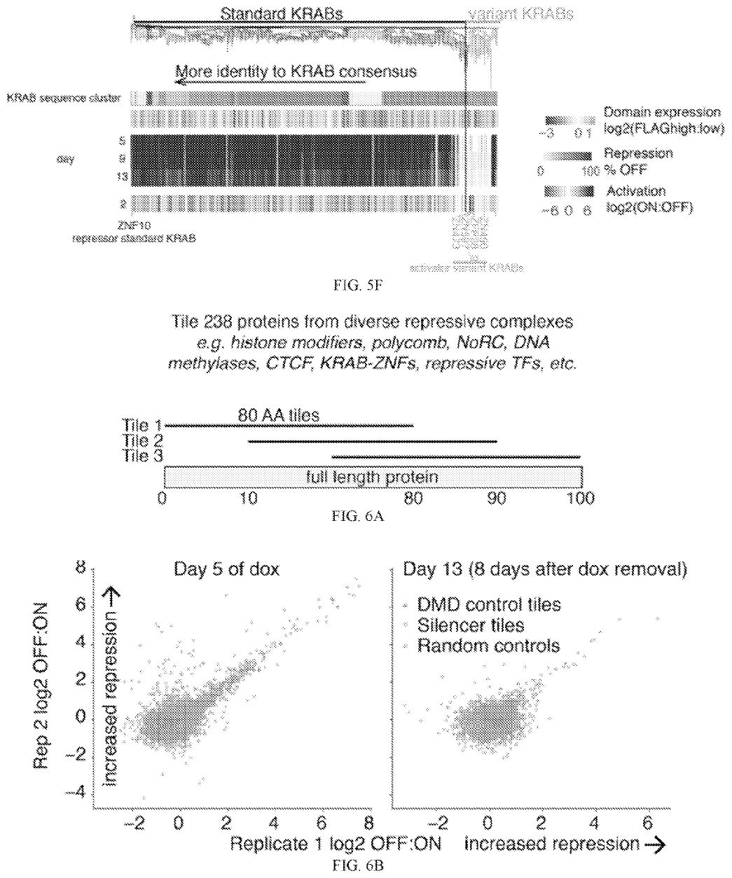

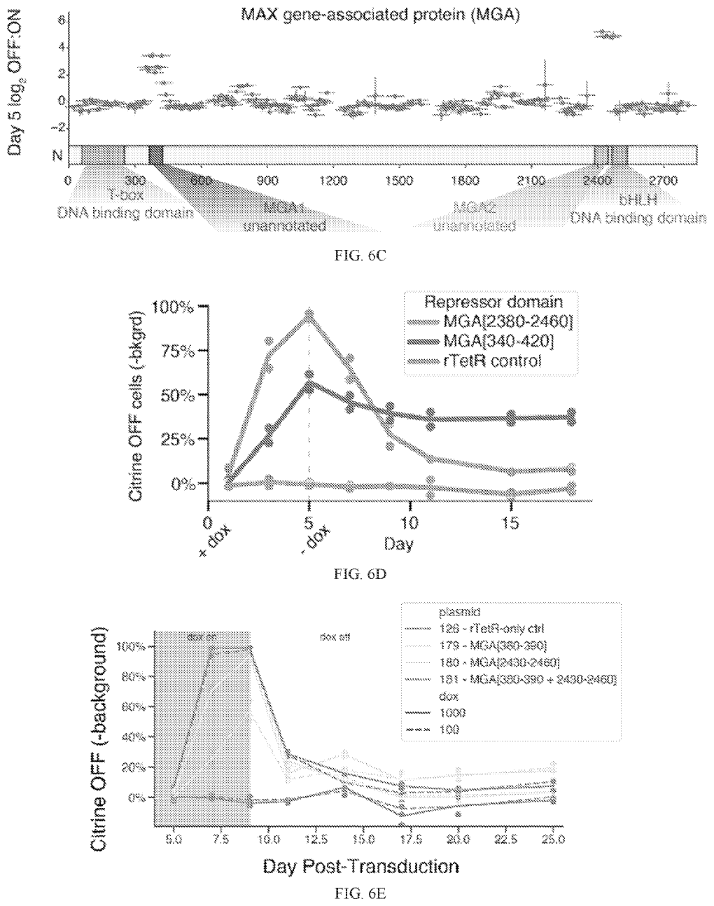

The protein domain is identified as a transcriptional activator when log 2 of the ratio of sequencing counts from reporter cells not having the surface marker, the fluorescent protein, or a combination thereof to sequencing counts from reporter cells having the surface marker, the fluorescent protein, or a combination thereof is at least two standard deviations from (e.g., less than) the mean of a negative control. (See FIG. 5B, for example).

### 3. Transcription Factors

The present disclosure also provides synthetic transcription factors comprising one or more transcriptional effector domains fused to a heterologous DNA binding domain. As used herein, the term “transcription factor” refers to a protein or polypeptide that interacts with, directly or indirectly, specific DNA sequences associated with a genomic locus or gene of interest to block or recruit RNA polymerase activity to the promoter site for a gene or set of genes.

In some embodiments the synthetic transcription factor comprises one or more transcriptional activator domains, one or more transcriptional repressor domains, or a combination thereof fused to a heterologous DNA binding domain. In some embodiments, the at least one of the one or more transcriptional activator domains or at least one of the one or more transcriptional repressor domains comprises an amino acid sequence having at least 70% (e.g., at least 70%, at least 75%, at least 80%, at least 85%, at least 90%, at least 95%, at least 98%, 99%) identity to any of SEQ ID NOs: 1-896. In some embodiments, at least one of the one or more transcriptional activator domains or at least one of the one or more transcriptional repressor domains comprises an amino acid sequence having at least 70% identity to any of SEQ ID NOs: 1346-1401.

In some embodiments, the one or more transcriptional activator domains, one or more transcriptional repressor domains, or a combination thereof are selected from those found in any of Tables 1 to 7.

In some embodiments, the one or more transcriptional activator domains, one or more transcriptional repressor domains, or a combination comprises an amino acid sequence having at least 70% identity to any of SEQ ID NOs: 5, 6, 7, 8, 13, 14, 35, 36, 37, 38, 41, 42, 67, 68, 81, 82, 83, 84, 85, 86, 91, 92, 99, 100, 107, 108, 109, 110, 119, 120, 123, 124, 135, 136, 141, 142, 153, 154, 167, 168, 169, 170, 177, 178, 183, 184, 193, 194, 203, 204, 205, 206, 211, 212, 229, 230, 237, 238, 241, 242, 259, 260, 271, 272, 281, 282, 283, 284, 285, 286, 293, 294, 325, 326, 327, 328, 329, 330, 333, 334, 347, 348, 349, 350, 355, 356, 363, 364, 365, 366, 379, 380, 387, 388, 391, 392, 395, 396, 401, 402, 409, 410, 413, 414, 415, 416, 421, 422, 425, 426, 437, 438, 441, 442, 457, 458, 467, 468, 489, 490, 499, 500, 509, 510, 513, 514, 523, 524, 525, 526, 527, 528, 539, 540, 541, 542, 547, 548, 563, 564, 565, 566, 567, 568, 569, 570, 571, 572, 577, 578, 579, $80, 583, 584, 585, 586, 593, 594, 597, 598, 599, 600, 601, 602, 605, 606, 613, 614, 615, 616, 623, 624, 627, 628, 633, 634, 639, 640, 643, 644, 647, and 648. In some embodiments, the one or more transcriptional activator domains, one or more transcriptional repressor domains, or a combination comprises an amino acid sequence selected from the group consisting of SEQ ID NOs: 5, 6, 7, 8, 13, 14, 35, 36, 37, 38, 41, 42, 67, 68, 81, 82, 83, 84, 85, 86, 91, 92, 99, 100, 107, 108, 109, 110, 119, 120, 123, 124, 135, 136, 141, 142, 153, 154, 167, 168,169, 170, 177, 178, 183, 184, 193, 194, 203, 204, 205, 206, 211, 212, 229, 230, 237, 238, 241, 242, 259, 260, 271, 272, 281, 282, 283, 284, 285, 286, 293, 294, 325, 326, 327, 328, 329, 330, 333, 334, 347, 348, 349, 350, 355, 356, 363, 364, 365, 366, 379, 380, 387, 388, 391, 392, 395, 396, 401, 402, 409, 410, 413, 414, 415, 416, 421, 422, 425, 426, 437, 438, 441, 442, 457, 458, 467, 468, 489, 490, 499, 500, 509, 510, 513, 514, 523, 524, 525, 526, 527, 528, 539, 540, 541, 542, 547, 548, 563, 564, 565, 566, 567, 568, 569, 570, 571, 572, 577, 578, 579, 580, 583, 584, 585, 586, 593, 594, 597, 598, 599, 600, 601, 602, 605, 606, 613, 614, 615, 616, 623, 624, 627, 628, 633, 634, 639, 640, 643, 644, 647, and 648.

In some embodiments, the one or more transcriptional activator domain, the one or more transcriptional repressor domain, or combination thereof is identified by the methods disclosed herein.

In some embodiments, the synthetic transcription factor comprises two or more transcription effector domains (e.g., transcriptional activator domains, transcriptional repressor domains, or a combination thereof) fused to a heterologous DNA binding domain. In some embodiments, the synthetic transcription factor comprises two or more transcriptional activator domains or two or more transcriptional repressors domains fused to a heterologous DNA binding domain. The two or more effector domains can be fused to the DNA binding domain in any orientation, and may be separated from each other with an amino acid linker. In select embodiments, the synthetic transcription factor comprises two or more transcription effector domains (e.g., transcriptional activator domains, transcriptional repressor domains, or a combination thereof) fused to a heterologous DNA binding domain.

In some embodiments, when the synthetic transcription factor comprises more than one transcription effector domains, the synthetic transcription factor may comprise at least one transcriptional activator domain or at least one transcriptional repressor domain as disclosed herein with at least one additional effector domain known in the art. See for example, Tycko J. et al., Cell. 2020 Dec. 23; 183 (7): 2020-2035, incorporated herein by reference in its entirety. In some embodiments, the one or more transcriptional activator domain, the one or more transcriptional repressor domain is identified by the methods described herein.

In some embodiments, when the synthetic transcription factor comprises more than one transcription effector domains, at least one of the one or more transcriptional activator domains comprises an amino acid sequence having at least 70% identity to any of SEQ ID NOs: 563-664. In some embodiments, at least one of the one or more transcriptional activator domains comprises an amino acid sequence having at least 70% identity to any of SEQ ID NOs: 563-596. In some embodiments, at least one of the one or more transcriptional activator domain is selected from those found in Table 2.

In some embodiments, the one or more transcriptional activator domains comprises an amino acid sequence having at least 70% identity to any of SEQ ID NOs: 563, 564, 565, 566, 575, 576, 579, and 580.

In some embodiments, the one or more transcriptional activator domains comprise: SEQ ID NO: 563 or SEQ ID NO: 564; SEQ ID NO: 565 or SEQ ID NO: 566; SEQ ID NO: 575 or SEQ ID NO: 576; SEQ ID NO: 579 or SEQ ID NO: 580; or a combination thereof. In some embodiments, the one or more transcriptional activator domains comprises two or more of SEQ ID NOs: 563, 565, 575, and 579. For example, the one or more transcriptional activator domains may include SEQ ID NOs: 563 and 565; SEQ ID NOs: 563 and 575; SEQ ID NOs: 563 and 579; SEQ ID NOs: 565 and 575; SEQ ID NOs: 565 and 579; or SEQ ID NOs: 575 and 579.

The one or more transcriptional activator domains may comprise three of more of SEQ ID NOs: 563, 565, 575, and 579. In some embodiments, the one or more transcriptional activator domains comprise SEQ ID NOs: 563, 565, and 579. In some embodiments, the one or more transcriptional activator domains comprise SEQ ID NOs: 563, 565, 575, and 579.

In some embodiments, the synthetic transcription factor comprises an amino acid sequence having at least 70% identity to any of SEQ ID NOs: 1331-1344. In some embodiments, the synthetic transcription factor comprises an amino acid sequence of any of SEQ ID NOs: 1331-1344.

In some embodiments, when the synthetic transcription factor comprises more than one transcription effector domains, at least one of the one or more transcriptional repressor domains comprises an amino acid sequence having at least 70% identity to any of SEQ ID NOs: 1-562 and 665-896. In some embodiments, when the synthetic transcription factor comprises more than one transcription effector domains, at least one of the one or more transcriptional repressor domains comprises an amino acid sequence having at least 70% identity to any of SEQ ID NOs: 1346-1401. In some embodiments, at least one of the one or more transcriptional repressor domains comprises an amino acid sequence having at least 70% identity to any of SEQ ID NO: 666. In some embodiments, at least one of the one or more transcriptional repressor domains is selected from those found in any of Tables 1, 3, 4 or 6.

In some embodiments, the one or more transcriptional repressor domains comprise an amino acid sequence having at least 70% identity to any of SEQ ID NOs: 32, 36, 363, or a combination thereof. In some embodiments, the one or more transcriptional repressor domains comprises SEQ ID NO: 32. In some embodiments, the one or more transcriptional repressor domains comprises SEQ ID NO: 36. In some embodiments, the one or more transcriptional repressor domains comprises SEQ ID NO: 363. In some embodiments, the synthetic transcription factor comprises at least one transcriptional repressor domains comprising an amino acid sequence having at least 70% identity to any of SEQ ID NOs: 32, 36, or 363, and at least one additional transcriptional repressor domain. The at least one additional transcriptional repressor domain may comprise any additional repressor domain, disclosed herein or known in the art. In some embodiments, the at least one additional transcriptional repressor domain comprises SEQ ID NO: 1347. In some embodiments, the synthetic transcription factor comprises an amino acid sequence of SEQ ID NO: 1345.

The DNA binding domain is any polypeptide which is capable of binding double- or single-stranded DNA, generally or with sequence specificity. DNA binding domains include those polypeptides having helix-turn-helix motifs, zinc fingers, leucine zippers, HMG-box (high mobility group box) domains, winged helix region, winged helix-turn-helix region, helix-loop-helix region, immunoglobulin fold, B3 domain, Wor3 domain, TAL effector DNA-binding domain and the like. The heterologous DNA binding domains may be a natural binding domain. In some embodiments, the heterologous DNA binding domain comprises a programmable DNA binding domain, e.g., a DNA binding domain engineered, for example by altering one or more amino acid of a natural DNA binding domain to bind to a predetermined nucleotide sequence.

In some embodiments, the DNA binding domain is capable of binding directly to the target DNA sequences.

The DNA-binding domain may be derived from domains found in naturally occurring Transcription activator-like effectors (TALEs), such as AvrBs3, Hax2, Hax3 or Hax4 (Bonas et al. 1989. Mol Gen Genet 218 (1): 127-36; Kay et al. 2005 Mol Plant Microbe Interact 18 (8): 838-48). TALEs have a modular DNA-binding domain consisting of repetitive sequences of residues; each repeat region consists of 34 amino acids. A pair of residues at the 12th and 13th position of each repeat region determines the nucleotide specificity and combining of the regions allows synthesis of sequence-specific TALE DNA-binding domains. In some embodiments, the TALE DNA binding domains may be engineered using known methods to provide a DNA binding domain with chosen specificity for any target sequence. The DNA binding domain may comprise multiple (e.g., 2, 3, 4, 5, 6, 10, 20, or more) Tal effector DNA-binding motifs. In particular, any number of nucleotide-specific Tal effector motifs can be combined to form a sequence-specific DNA-binding domain to be employed in the present transcription factor.

In some embodiments, the DNA binding domain associates with the target DNA in concert with an exogenous factor.

In some embodiments, the DNA binding domain is derived from a Clustered Regularly Interspaced Short Palindromic Repeats associated (Cas) protein (e.g., catalytically dead Cas9) and associates with the target DNA through a guide RNA. The gRNA itself comprises a sequence complementary to one strand of the DNA target sequence and a scaffold sequence which binds and recruits Cas9 to the target DNA sequence. The transcription factors described herein may be useful for CRISPR interference (CRISPRi) or CRISPR activation (CRISPRa).

The guide RNA (gRNA) may be a crRNA, crRNA/tracrRNA (or single guide RNA, sgRNA). The gRNA may be a non-naturally occurring gRNA. The terms “gRNA,” “guide RNA” and “guide sequence” may be used interchangeably throughout and refer to a nucleic acid comprising a sequence that determines the binding specificity of the Cas protein. A gRNA hybridizes to (complementary to, partially or completely) the DNA target sequence.

The gRNA or portion thereof that hybridizes to the target nucleic acid (a target site) may be any length necessary for selective hybridization. gRNAs or sgRNA(s) can be between about 5 and about 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length, or longer).

To facilitate gRNA design, many computational tools have been developed (See Prykhozhij et al. (PLOS ONE, 10 (3): (2015)); Zhu et al. (PLOS ONE, 9 (9) (2014)); Xiao et al. (Bioinformatics. January 21 (2014)); Heigwer et al. (Nat Methods, 11 (2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

The present disclosure also provides synthetic transcription factors comprising one or more transcriptional effector domains fused to an exogenous factor which associates with a second exogenous factor comprising a DNA binding domain. Such inducible systems include, but not limited to, tetracycline Tet/DOX inducible systems, light inducible systems, Abscisic acid (ABA) inducible systems, cumate systems, 40HT/estrogen inducible systems, ecdysone-based inducible systems, and FKBP12/FRAP (FKBP12-rapamycin complex) inducible systems.

The present disclosure also provides nucleic acids encoding a synthetic transcription factor or a transcriptional effector (e.g., activator or repressor) domain, as disclosed herein. For example, the effector domains may be encoded by nucleic acids disclosed in Tables 1-3. In some embodiments, the effector domains may be encoded by nucleic acids having at least 70% identity to any of SEQ ID NOs: 897-1329. In some embodiments, the nucleic acid encodes one or more synthetic transcription factor or one or more effector domain.

Nucleic acids of the present disclosure can comprise any of a number of promoters known to the art, wherein the promoter is constitutive, regulatable, or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

To construct cells that express the present transcription factors, expression vectors for stable or transient expression of the present system may be constructed via conventional methods and introduced into cells. For example, nucleic acids encoding the components the disclose transcription factors, or other nucleic acids or proteins, may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA.

Thus, the disclosure further provides for cells comprising a synthetic transcription factor, a nucleic acid, or a vector, as disclosed herein.

Conventional viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1): 33-40; and Walther W. and Stein U., 2000 Drugs, 60(2): 249-71, incorporated herein by reference.

The nucleic acids or transcription factors may be delivered by any suitable means. In certain embodiments, the nucleic acids or proteins thereof are delivered in vivo. In other embodiments, the nucleic acids or proteins thereof are delivered to isolated/cultured cells in vitro or ex vivo to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83), incorporated herein by reference.

As such, the disclosure provides an isolated cell comprising the vector(s) or nucleic acid(s) disclosed herein. Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14:810-817 (1993); Lucklow, Curr. Opin. Biotechnol., 4:564-572 (1993); and Lucklow et al., J. Virol., 67:4566-4579 (1993), incorporated herein by reference. Desirably, the cell is a mammalian cell, and in some embodiments, the cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines.

Methods for selecting suitable mammalian cells and methods for transformation, culture, amplification, screening, and purification of cells are known in the art.

The present invention is also directed to compositions or systems comprising a synthetic transcription factor, a nucleic acid, a vector, or a cell, as described herein. In some embodiments, the compositions or system comprises two or more synthetic transcription factors, nucleic acids, vectors, or cells.

In some embodiments, the composition or system further comprises a gRNA. The gRNA may be encoded on the same nucleic acid as a synthetic transcription factor or a different nucleic acid. In some embodiments, the vector encoding a synthetic transcription factor may further encode a gRNA, under the same or different promoter. In some embodiments, the gRNA is encoded on its own vector, separated from that of the transcription factor.

### 4. Methods of Modulating Gene Expression

The present disclosure also provides methods of modulating the expression of at least one target gene in a cell, the method comprising introducing into the cell at least one synthetic transcription factor, nucleic acid, vector, or composition or system as described herein. In some embodiments, the gene expression of at least two genes is modulated.

In some embodiments, the gene is an endogenous gene. In some embodiments, the gene is an exogenous gene. In some embodiments, the gene is on an exogenous vector. In some embodiments, the exogenous gene was introduced into the cell as part of a gene therapy regime. For example, a controllable and activatable vector expressing secreted hepatocyte growth factor has broad therapeutic potential due to its capacity to induce regeneration of health tissues when transduced into the tissue or interest or neighboring tissues (e.g., liver to regenerate damaged liver or kidney, heart for prevention of/and regeneration after heart attack, brain for neurogenesis in Alzheimer's and Parkinson's diseases).

Modulation of expression comprises increasing or decreasing gene expression compared to normal gene expression for the target gene. When the gene expression of at least two genes is modulation, both genes may have increased gene expression, both gene may have decreased gene expression, or one gene may have increased gene expression and the other may have decreased gene expression.

The cell may be a prokaryotic or eukaryotic cell. In preferred embodiments, the cell is a eukaryotic cell. In some embodiments, the cell is in vitro. In some embodiments, the cell is ex vivo.

In some embodiments, the cell is in an organism or host, such that introducing the disclosed systems, compositions, vectors into the cell comprises administration to a subject. The method may comprise providing or administering to the subject, in vivo, or by transplantation of ex vivo treated cells, at least one synthetic transcription factor, nucleic acid, vector, or composition or system as described herein.

A “subject” may be human or non-human and may include, for example, animal strains or species used as “model systems” for research purposes, such a mouse model as described herein. Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing,” “administering,” “introducing,” are used interchangeably herein and refer to the placement of the systems of the disclosure into a subject by a method or route which results in at least partial localization of the system to a desired site. The systems can be administered by any appropriate route which results in delivery to a desired location in the subject.

### 5. Kits

Also within the scope of the present disclosure are kits including at least one or all of at least one nucleic acid encoding an effector domain, or a DNA binding domain, or a combination thereof, at least one synthetic transcription factor, or nucleic acid encoding thereof, vectors encoding at least one effector domain or at least one synthetic transcription factor, a composition or system as described herein, a cell comprising an effector domain, a DNA binding domain, a synthetic transcription factor, or a nucleic acid encoding any of thereof, a reporter cell as described herein and a two-part reporter gene as described herein or a nucleic acid encoding thereof.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may include instructions for use in any of the methods described herein. The instructions can comprise a description of use of the components for the methods of identifying repressor domains or methods of modulating gene expression.

The kits provided herein are in suitable packaging. Suitable packaging includes, but is not limited to, vials, bottles, jars, flexible packaging, and the like.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

The present disclosure also provides for kits for performing the methods or producing the components in vitro. The kit may include the components of the present system. Optional components of the kit include one or more of the following: (1) buffer constituents, (2) control plasmid, (3) sequencing primers.

### 6. Examples

Human gene expression is regulated by thousands of proteins that activate or repress transcription. We lack a complete and quantitative description of these proteins' effector domains, the domains sufficient to mediate changes in gene expression. To systematically measure transcriptional effector domains in human cells, provided herein is a high-throughput assay in which libraries of protein domains are fused to a DNA-binding domain and recruited to a reporter gene. The cells are then separated by reporter expression level and the library of protein domains is sequenced. The reporter is a synthetic surface marker that facilitates simple separation of tens of millions of cells into high- and low-expression populations, using magnetic beads.

Gene silencing and epigenetic memory was quantified after recruitment of all nuclear protein domains of ≤80 amino acids. Using the measurements for the complete families of >300 KRAB domains and >200 Homeodomains, relationships were discovered between transcription factor's repressor domain strength and their evolutionary history and developmental role. Further, a deep mutational scan of the ZNF10 KRAB effector function and identified substitutions with enhanced stability and repression compared to the KRAB domain used in CRISPRi. To search for effector domains beyond previously annotated regions, the sequence of 238 repressor complex proteins was tiled and novel repressor domains as short as 10 amino acids were discovered in unannotated regions of large chromatin regulators, including the non-canonical polycomb 1.6 recruitment protein MGA. Greater than 20 repressors were individually characterized and all of them were found to silence a reporter gene in an all-or-nothing fashion at the single-cell level, but with distinct dynamics of silencing and epigenetic memory.

In addition, new activator domains in nuclear proteins were discovered, including a highly divergent acidic KRAB domain variant.

Together, these results demonstrate a strategy for systematic measurement of transcriptional effector domain activity in human cells, and expand the number of compact transcriptional effector domains that can be applied in synthetic transcription and epigenetic perturbation technologies.

Problems addressed by the present technology:
i. Unknown which genes have effector function
ii. Within known TF/CR genes, often unknown which domains have this function
iii. Within domain families including known effector domains, unknown which family members have this function
iv. Within known effector domains, unknown which residues are necessary, and how mutations reduce or enhance function

The systems and methods provided herein can measure regulatory domains of activators and repressors capability to change the output from a reporter promoter. Historically, this requires low throughput work so relatively few effector domains have been measured. The systems and methods provided herein off an alternative high-throughput assay.

The systems and methods find use, for example, for: a. understanding gene regulation, predicting function of non-coding regulatory elements that these proteins bind to; and b. identifying effector domains for epigenome perturbation tools.

Previously, a limited number of transcriptional effector domains were available for the engineering of synthetic transcription factors. To address this limitation, provided herein is a high-throughput approach to screening and quantifying the function of transcriptional effectors domains. This approach enabled the discovery of hundreds of effector domains that can upregulate or downregulate transcription in a targeted manner when fused onto a DNA binding domain. This process also identifies mutants of effector domains with enhanced activity. These effector domains can be used to engineer synthetic transcription factors for applications in gene and cell therapy, synthetic biology, and functional genomics.

The new transcriptional effector domains provided herein have several advantages for applications that rely on synthetic transcription factors. We identify short domains (≤80 amino acids) and a high-throughput process for shortening them further to the minimally sufficient sequence, which is an advantage for delivery (e.g., packaging in viral vectors). In some cases, we identify potent effector domains that are as short as 10 amino acids. The domains are extracted from human proteins, which provides the advantage of reducing immunogenicity in comparison to viral effector domains. Most of these domains have not been reported as transcriptional effectors previously.

By performing high-throughput recruitment with the Pfam domain library against both a strong pEF promoter and a weak minCMV promoter, both repressor and activator domains were able to be measured. One possible reason that many more repressors were found is that they are more often autonomous stably-folding sequences which meet the Pfam definition of a domain while TADs are more often disordered or low-complexity regions that are not annotated as domains. Another possible reason could be that co-activators are more limiting in the nucleus than co-repressors (Gillespie Mol Cell 2020), which implies lower expression of activator domains could result in greater activation strength, but this effect would not be expected to completely mask signal in the screen. New library designs that tile transcription factors or focus on regions with TAD-like signatures (e.g., acidity) will uncover additional activator domains.

In addition, a high-throughput process for testing mutations in these domains in order to identify enhanced variants is disclosed herein. The high-throughput approach is more readily enabled by development of an artificial cell surface marker that provides more efficient, inexpensive, and rapid screening of these libraries using magnetic separation. This is an advantage over the more conventional approach of sorting libraries based on fluorescent reporter gene expression.

### Example 1

### HT-Recruit Identifies Hundreds of Repressor Domains in Human Proteins

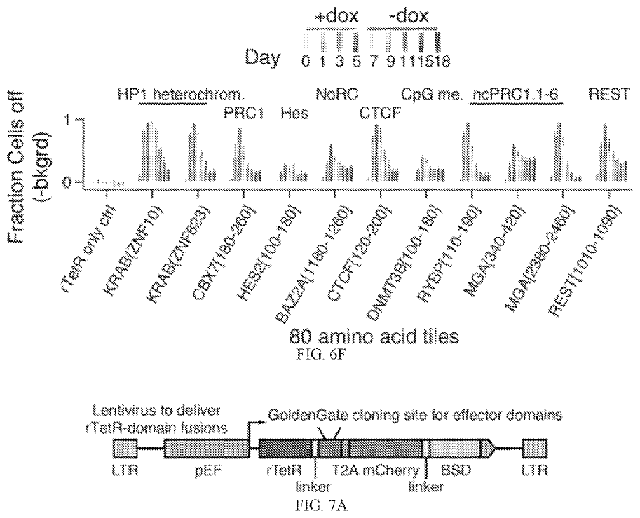

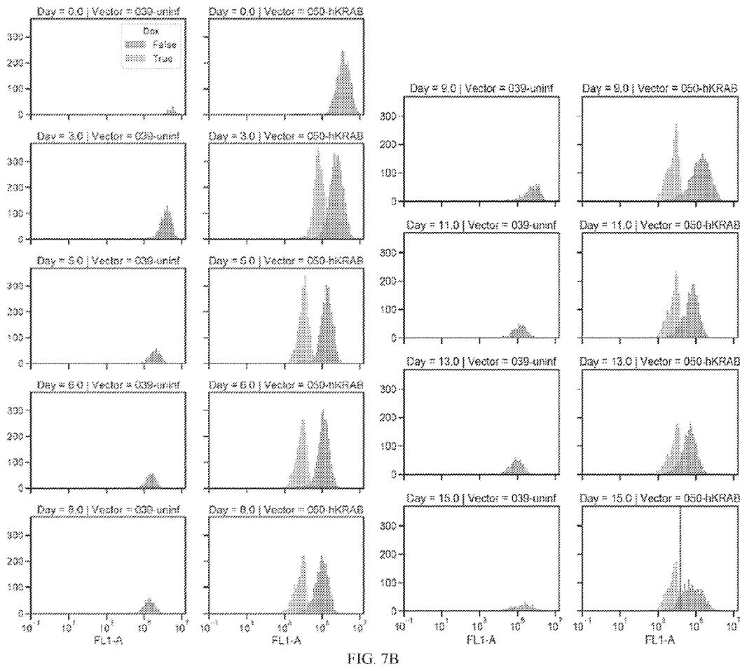

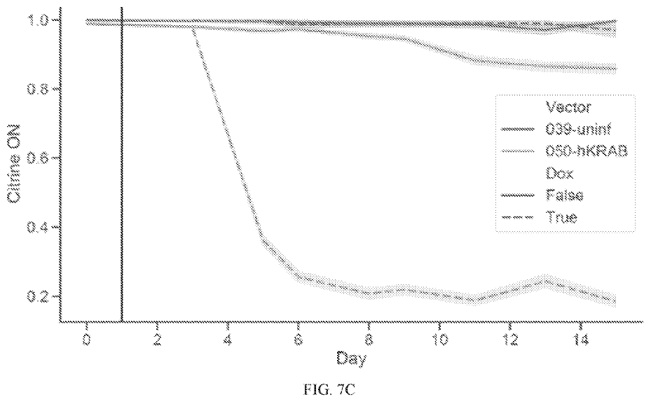

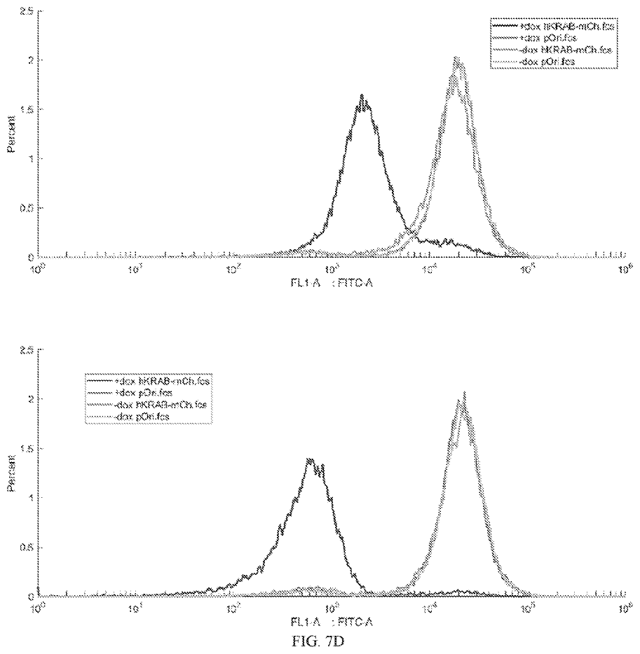

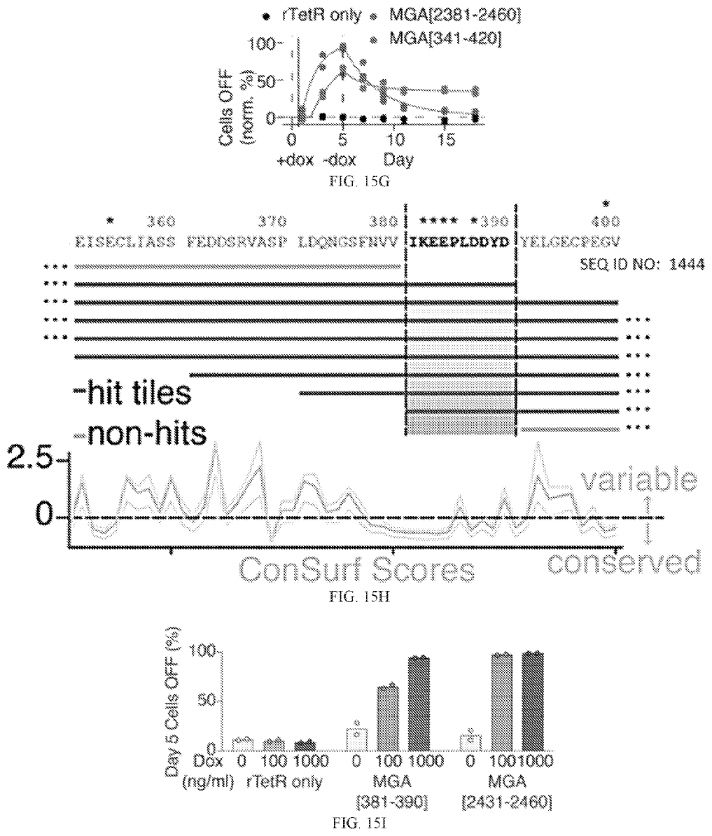

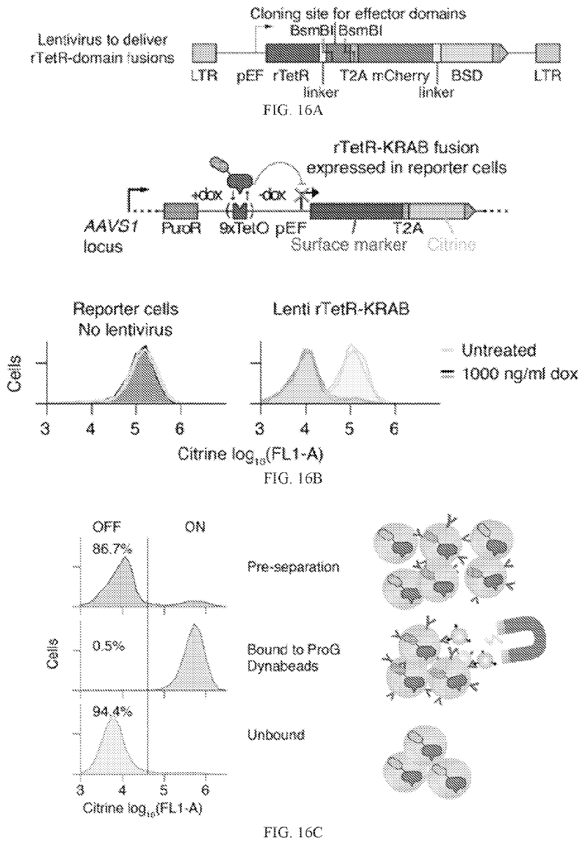

In order to turn the classical recruitment reporter assay into a high-throughput assay of transcriptional domains, two problems were solved: (1) modification of the reporter to make it compatible with rapid screening of libraries of tens of thousands of domains, and (2) development of a strategy to generate a library of candidate effector domains. To improve on the previously published fluorescent reporter (Bintu et al., 2016), a synthetic surface marker was engineered to enable facile magnetic separation of large numbers of cells and the reporter was integrated in a suspension cell line amenable to cell culture in large volume spinner flasks. Specifically, K562 reporter cells with 9×TetO binding sites upstream of a strong constitutive pEF1a promoter that drives expression of a two-part reporter consisting of a synthetic surface marker (the human IgG1 Fc region linked to an Igk leader and PDGFRβ transmembrane domain) and a fluorescent citrine protein (FIG. 1) were generated. Flow cytometry confirmed that recruitment of a known repressor domain, the KRAB domain from the zinc finger transcription factor ZNF10, at the TetO sites silenced this reporter in a doxycycline-dependent manner within 5 days (FIGS. 7 and 16A and 16B). Magnetic separation with ProG Dynabeads that bind to the synthetic surface marker separated cells with the reporter ON from OFF cells (FIGS. 7 and 16C).

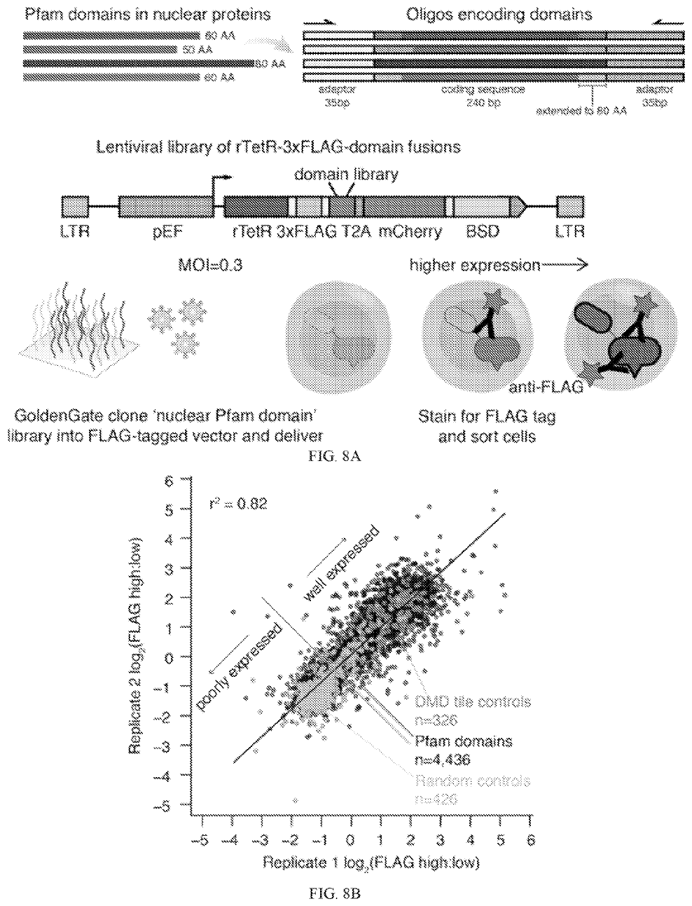

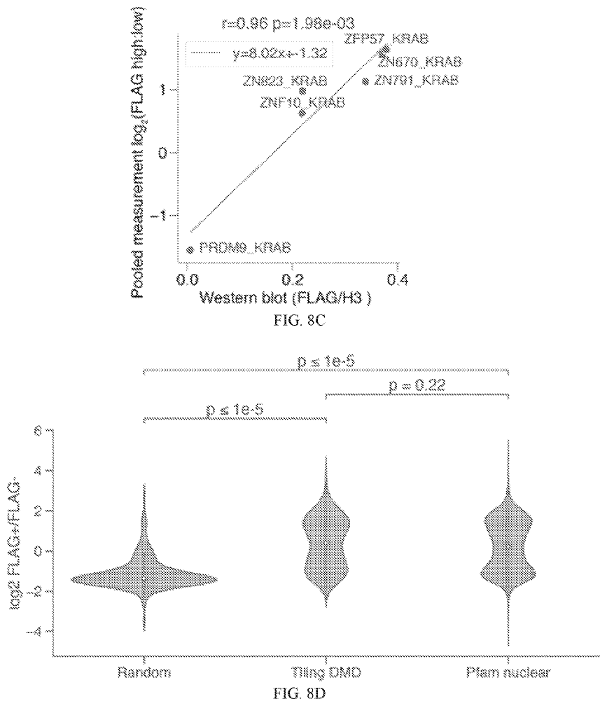

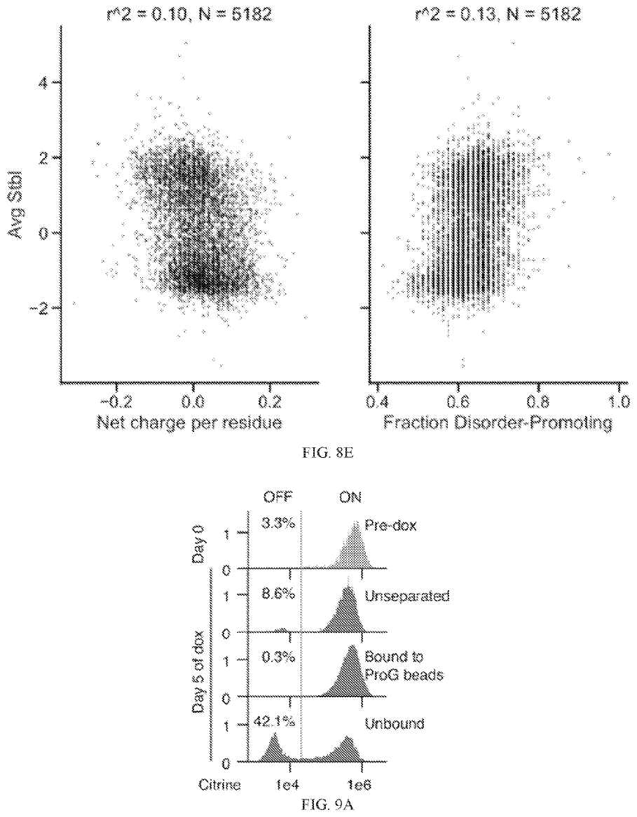

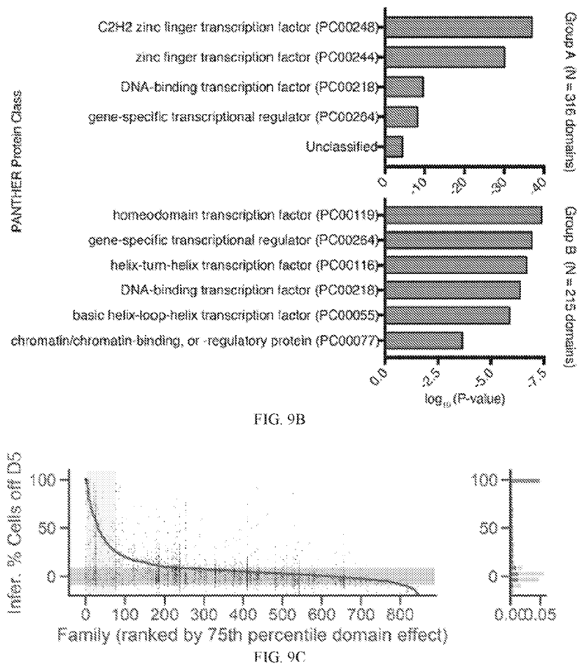

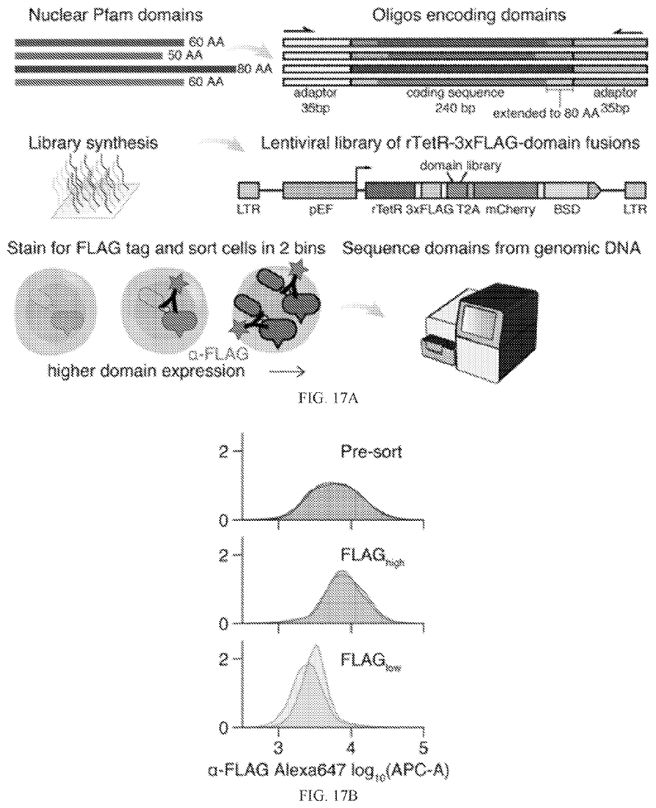

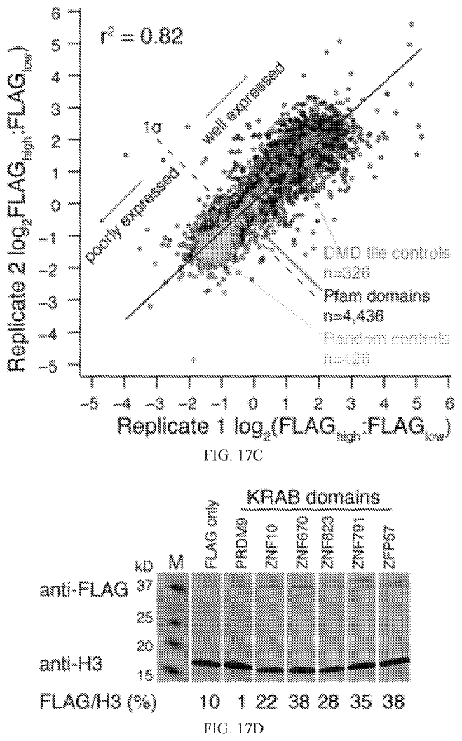

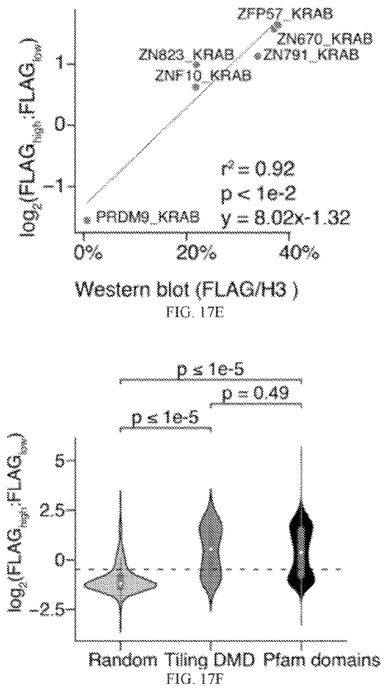

Sequences were pulled from the UniProt database for Pfam-annotated domains in human proteins that can localize to the nucleus (including non-exclusively nuclear-localized proteins). In total, 14,657 domains were retrieved. Of these, 72% were less than or equal to 80 amino acids (AA) long (FIG. 1), which made them compatible with pooled synthesis as 300 base oligonucleotides. For domains shorter than 80 AA, the domain sequence was extended on both ends with the adjacent residues from the native protein sequence in order to reach a length of 80 AA and avoid PCR amplification biases. 861 negative controls that were either random 80 AA sequences or 80 AA sequences tiled along the DMD protein with a 10 AA tiling window were added. The DMD protein was not localized in the nucleus (Chevron et al., 1994), and thus unlikely to feature domains with transcriptional activity. The library was cloned for lentiviral expression as a fusion protein with either the rTetR doxycycline-inducible DNA-binding domain alone, or with a 3×-FLAG-tagged rTetR (FIGS. 17A and 8) and delivered to K562 reporter cells (FIG. 1).

Before assaying for transcriptional activity, it was determined which protein domains were well-expressed in K562 cells using a high-throughput approach (FIGS. 17A and 8). The library of cells was stained with an anti-FLAG fluorescent-labeled antibody, sorted the cells into two bins (FIGS. 17B and 8), genomic DNA was extracted, and the frequency of each domain by amplicon sequencing was counted. The sequencing counts were used to compute the enrichment ratio in the FLAGhigh versus FLAGlow population for each domain, as a measure of expression level. These measurements were reproducible between separately transduced biological replicates (r²=0.82, FIGS. 17C and 8), and highly correlated with individual domain fusion expression levels measured by Western blot (r²=0.92, FIGS. 17D and 17E and 8). Native Pfam domains were significantly better-expressed than the random sequence controls (p<1e-5, Mann Whitney test), while the Pfam domains and the DMD tiling controls were similarly well-expressed (FIGS. 17F and 8). A threshold was set to identify well-expressed domains with a FLAGhigh:FLAGlow ratio one standard deviation above the median of the random controls. By this definition, 66% of the Pfam domains were well-expressed; these domains were the focus of further analysis.

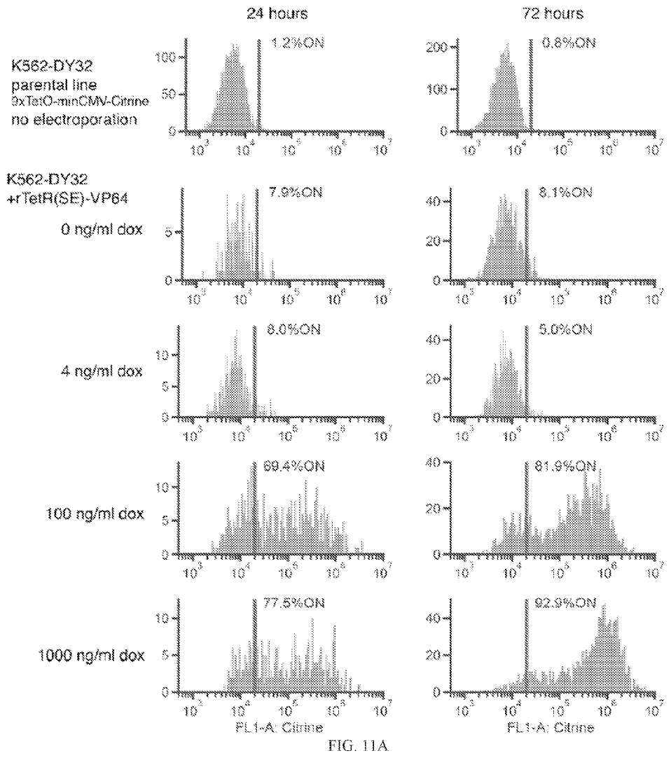

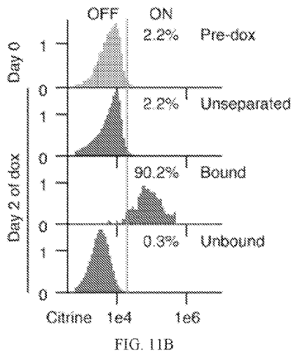

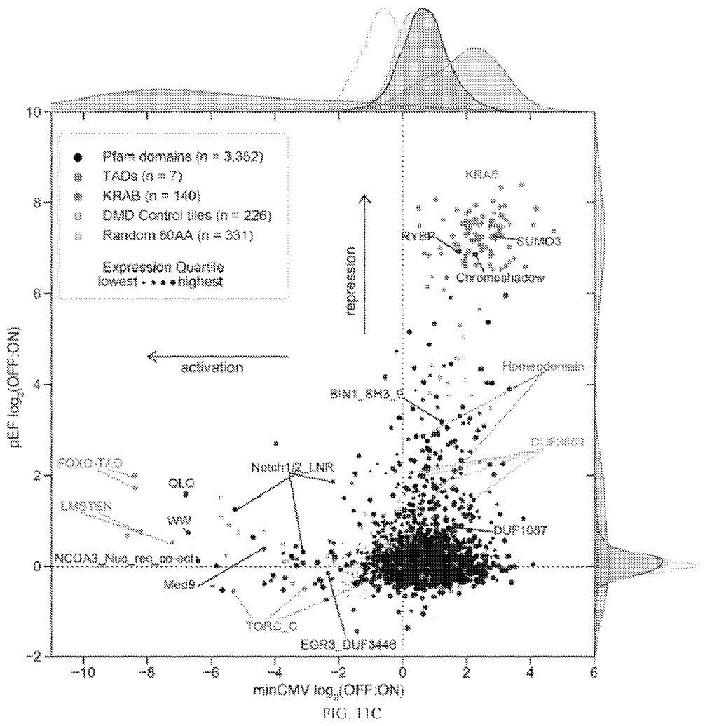

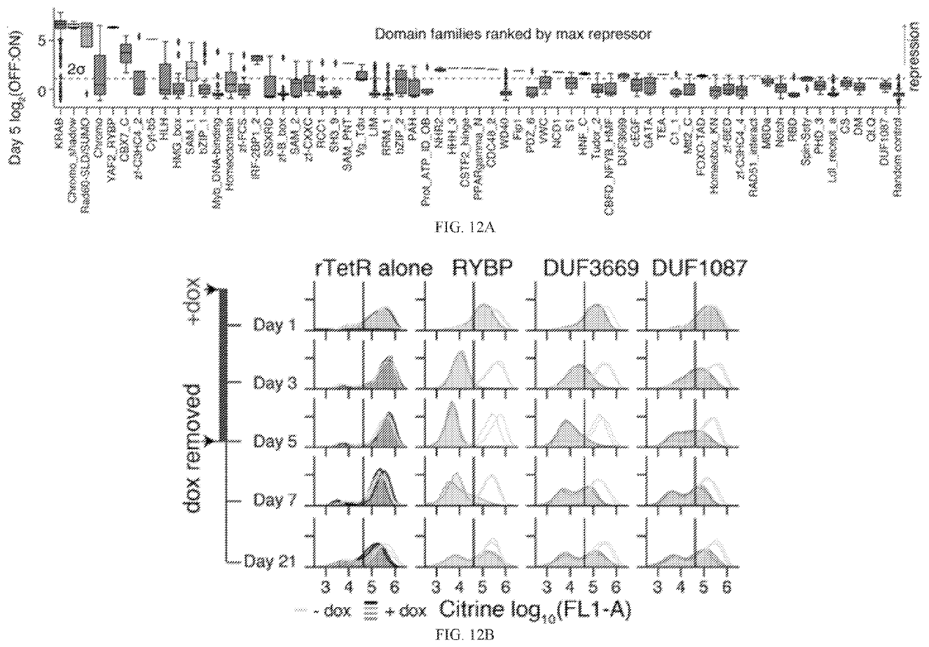

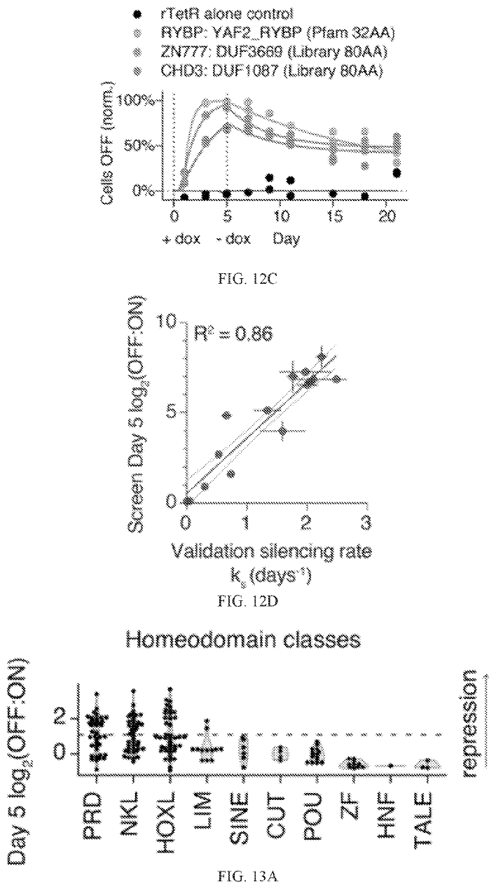

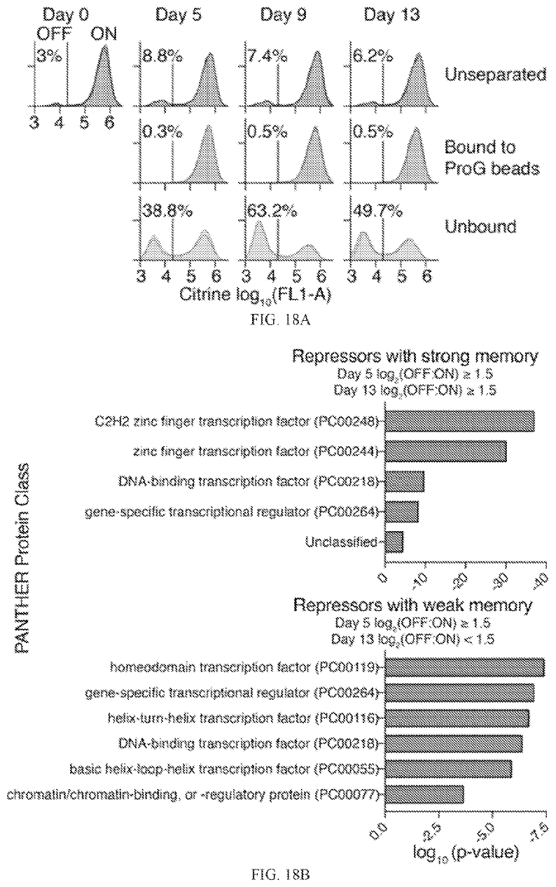

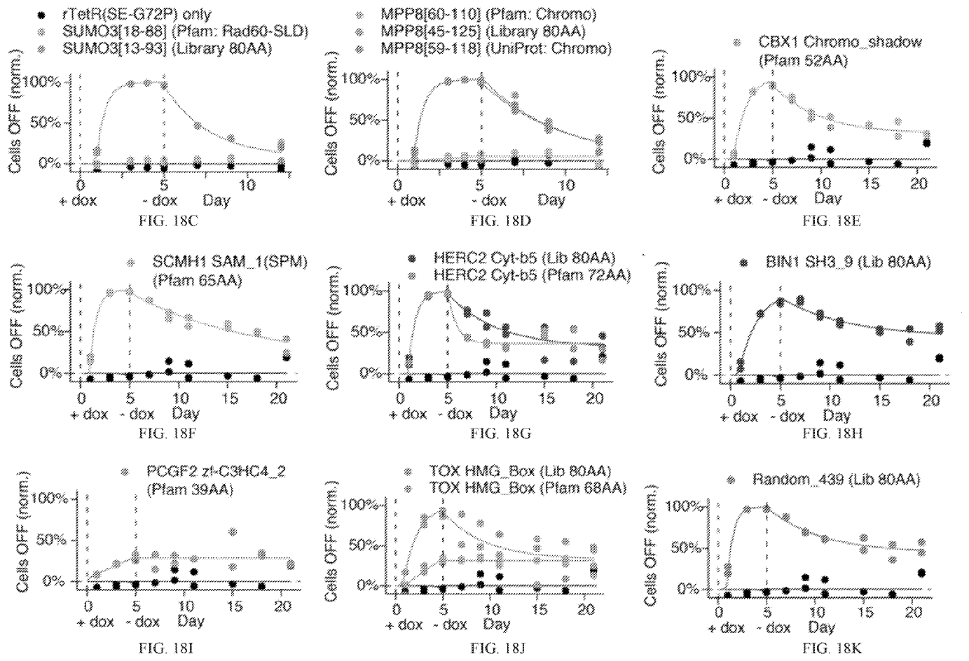

The Pfam domain library was screened for transcriptional repressors. The pooled library of cells was treated with doxycycline for 5 days, which gave sufficient time after transcriptional silencing for the reporter mRNA and protein to degrade and dilute out due to cell division, resulting in a clear bimodal mixture of ‘ON’ and ‘OFF’ cells (FIGS. 18A and 9). Then, magnetic cell separation (FIGS. 18A and 9) and domain sequencing were performed, then the log 2(OFF:ON) ratio was computed for each library member using the read counts in the unbound and bead-bound populations (FIG. 1). For clarity, the bead-bound population was referred to as ‘ON’ and the unbound population as ‘OFF’. The measurements were highly reproducible between separately transduced biological replicates (r²=0.96, FIG. 1). Domains were called as hits when they caused repression that was more than 2 standard deviations above the mean of the poorly expressed negative controls. This resulted in 446 repressor hits at day 5, with domains from 63 domain families (FIG. 12A). These repressor domains are found in 451 human proteins, because in some cases the exact same domain sequence occurs in multiple genes. Known repressor domains (e.g., KRAB from human ZNF10, Chromoshadow from CBX5) from 10 domain families described as repressors or co-repressor-binding domains by Pfam were among the hits. To measure epigenetic memory, additional time points were taken at days 9 and 13. The set of proteins containing hits was significantly enriched for transcription factors and chromatin regulators when compared to all nuclear proteins used in the library, but different categories of proteins were differentially enriched when classified by their memory levels (FIGS. 18B and 9). Specifically, the repressors with high memory (cells remaining OFF) at day 13 were most enriched for C2H2 zinc finger transcription factors which include KRAB ZNF proteins, and the repressors with low memory were most enriched for homeodomain transcription factors which include the Hox proteins. Overall, the very high reproducibility and identification of expected positive control repressor domains among the hits suggested the screening method, called HT-recruit, yielded reliable results. Amino acid and nucleic acid sequences for repressors identified in the nuclear Pfam domain library are shown in Table 1, with higher scores indicating increased repression.

One of the strongest hits was the YAF2_RYBP, a domain present in the RING1- and YY1-binding protein (RYBP) and its paralog YY1-associated Factor 2 (YAF2), which are both components of the polycomb repressive complex 1 (PRC1) (Chittock et al., 2017; García et al., 1999). The domain from the RYBP protein as annotated by Pfam (which is just 32 amino acids, thus shorter than the version synthesized in the 80 AA domain library) was individually tested and rapid silencing of the reporter gene was confirmed (FIG. 12B). RYBP-mediated silencing was also demonstrated in a recent report of full-length RYBP protein recruitment in mouse embryonic stem cells (Moussa et al., 2019; Zhao et al., 2020). The result established that the 32 AA RYBP domain, which has been shown by surface plasmon resonance to be the minimal required domain to bind the polycomb histone modifier enzyme RING1B (Wang et al., 2010), was sufficient to mediate silencing in cells.

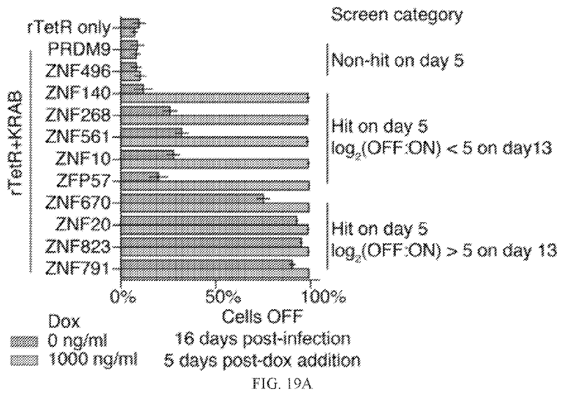

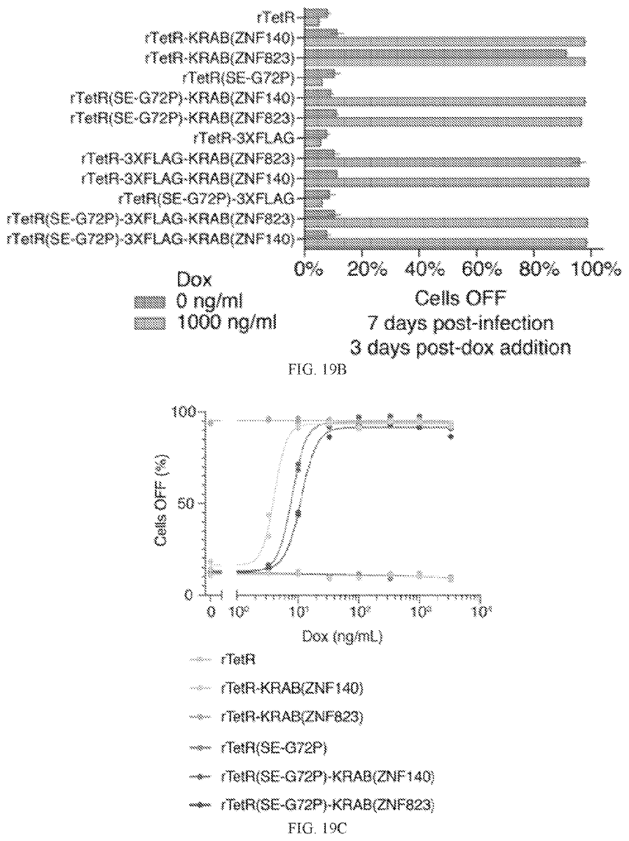

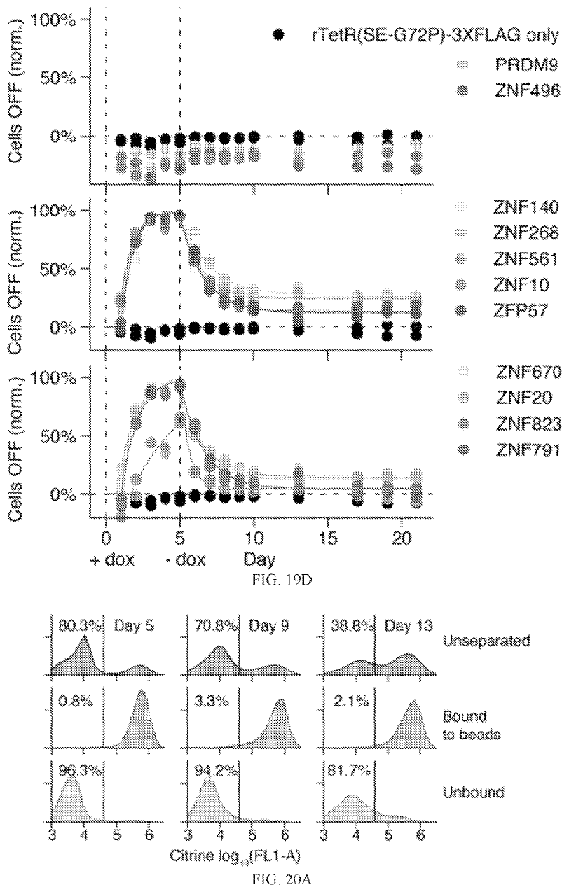

To quantify repression kinetics, the citrine level distributions were gated to calculate a percentage of silenced cells with normalization of the uniform low level of background silencing in the untreated cells, and then the data was fit to a model with an exponential silencing rate during doxycycline treatment and an exponential decay (or reactivation) after doxycycline removal that plateaus at a constant irreversibly silent percentage of cells (FIG. 12C). Using this approach, the repressor function of SUMO3, the Chromo domain from MPP8, the Chromoshadow domain from CBX1, and the SAM_1/SPM domain from SCMH1 (FIGS. 18C-18F and 9), which all had previous support for repressor function from recruitment or co-repressor binding assays, were also validated (Chang et al., 2011; Chupreta et al., 2005; Frey et al., 2016; Lechner et al., 2000). Silencing rates from all individual measurements (for the repressor hits above and the other hits discussed below, FIGS. 18C-18K and 9) correlated well with the high-throughput measurements of silencing at day 5 (R²=0.86, FIG. 12D). These individual validations were performed using a new variant of the DNA binding domain rTetR (SE-G72P) that was engineered to mitigate leakiness in the absence of doxycycline in yeast (Roney et al., 2016), and which was found to not leak in human cells (FIGS. 19A and 19B), making it a useful tool for mammalian synthetic biology. This new rTetR variant has the same silencing strength at maximum doxycycline recruitment as the original rTetR (FIG. 19C), which was also evidenced by the high correlation between individual validations and screen scores (FIG. 12D). Together, these validation experiments demonstrated that HT-recruit both successfully identified bona fide repressors and quantified the repression strength for each domain with accuracy comparable to individual flow cytometry experiments.

### Example 2

Identification of Domains of Unknown Function that Repress Transcription

Over 22% of the Pfam domain families are labeled as Domains of Unknown Function (DUFs), while others are not named using this label but are nevertheless DUFs (El-Gebali et al., 2019). These domains have recognizable sequence conservation but lack experimental characterization. As such, the high-throughput domain screen described herein offered the opportunity to associate initial functions with DUFs. First, DUF3669 domains were identified as repressor hits and individually validated by flow cytometry (FIGS. 12A-12C). These DUFs are natively found in KRAB zinc finger proteins, which is a gene family containing many repressive transcription factors. Concordant results demonstrating transcriptional repression after recruitment of two DUF3669 family domains were recently published (Al Chiblak et al., 2019), and the high-throughput results expand this finding to include the four remaining untested DUF3669 sequences. The HNF3 C-terminal domain, HNF_C, is another DUF, although it has a more specific name because it is only found in Hepatocyte Nuclear Factors 3 alpha and beta (also known as FOXA1 and 2). The HNF_C domains from both FOXA1 and 2 were also found as repressor hits. They both include a EH1 (engrailed homology 1) motif, characterized by the FxIxxIL sequence, that has been nominated as a candidate repressor motif (Copley, 2005).

All three of the IRF-2BP1_2 N-terminal zinc finger domains (Childs and Goodbourn, 2003), an uncharacterized domain found in the interferon regulatory factor 2 (IRF2) co-repressors IRF2BP1, IRF2BP2, and IRF2BPL, were repressor hits. The Cyt-b5 domain in the DNA repair factor HERC2 E3 ligase (Mifsud and Bateman, 2002) was another functionally uncharacterized domain that was validated as a strong repressor hit (FIGS. 18G and 9). The SH3_9 domain in BINI is a largely uncharacterized variant of the SH3 protein-binding domain, which was also validated as a repressor (FIGS. 18H and 9). BINI is a Myc-interacting protein and tumor suppressor (Elliott et al., 1999) that is also associated with Alzheimer's disease risk (Nott et al., 2019). Concordant with the results, both full-length BINI and a Myc-binding domain deletion mutant were previously shown to repress transcription in a Gal4 recruitment assay in HeLa cells (Elliott et al., 1999), and the BINI yeast homolog hob 1 has been linked to transcriptional repression and histone methylation (Ramalingam and Prendergast, 2007). In addition, the repressor activity of the HMG_box domain from the transcription factor TOX and of the zf-C3HC₄₋₂ RING finger domain from the polycomb component PCGF2 were validated (FIGS. 18I and 18J). Lastly, DUF1087 was found in CHD chromatin remodelers and, although its high-throughput measurement was just below the screen significance threshold (FIG. 12A), the CHD3 DUF1087 was validated as a weak repressor by individual flow cytometry (FIGS. 12B and 12C). Together, these results demonstrated that high-throughput protein domain screens can assign initial functions to DUFs and expand understanding of the functions of incompletely characterized domains.

### Example 3

A Random Sequence with Strong Repressor Activity

Random sequences have not previously been tested for repressor activity. Surprisingly, one of the random 80 AA sequences, which were designed as negative controls, was a strong repressor hit with an average log 2(OFF:ON)=4.0, despite having a weak expression level below the threshold. Individual validation by flow cytometry confirmed that this sequence fully silenced the population of reporter cells after 5 days of recruitment with moderate epigenetic memory up to two weeks after doxycycline removal (FIGS. 18K and 9). One additional random sequence showed a repression score marginally above the hit threshold.

### Example 4

### Repressor KRAB Domains are Found in Younger Proteins

The data provided an opportunity to analyze the function of all effector domains in the largest family of transcription factors: the KRAB domains. The KRAB gene family includes some of the strongest known repressor domains (such as the KRAB in ZNF10). Previous studies of a subset of repressive KRAB domains revealed that they can repress transcription by interacting with the co-repressor KAP1, which in turn interacts with chromatin regulators such as SETDB1 and HP1 (Cheng et al., 2014). However, it remains unclear how many of the KRAB domains are repressors, and whether the recruitment of KAP1 is necessary or sufficient for repression across all KRABs.

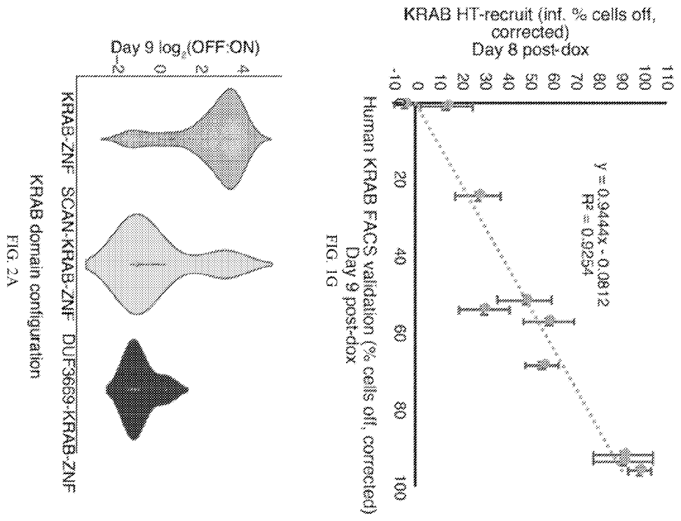

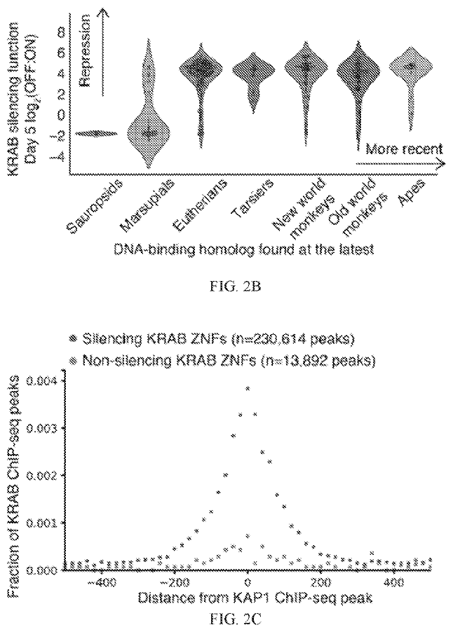

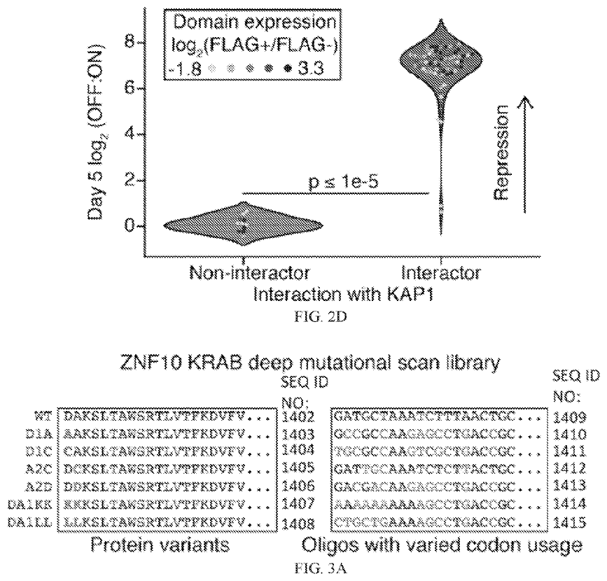

The library included 335 human KRAB domains, and 92.1% were found as repressor hits after filtering for domains that were well-expressed. 9 repressor hit and 2 non-hit KRAB domains were individually validated by flow cytometry and these categorizations were confirmed in every case (FIG. 19D). Then, the domain recruitment results were compared with previously published immunoprecipitation mass spectrometry data generated from full-length KRAB protein pulldowns (Helleboid et al., 2019) and all but one of the non-repressive KRABs were in proteins that do not interact with KAP1 (the one exceptional KRAB was lowly expressed), and all of the repressor hit KRAB domains were KAP1 interactors (p<1e-9, Fisher's exact test, FIG. 2). Furthermore, available ChiP-seq and ChIP-exo datasets was analyzed (ENCODE Project Consortium et al., 2020; Imbeault et al., 2017; Najafabadi et al., 2015; Schmitges et al., 2016) and repressive KRAB domains were from KRAB Zinc Finger proteins that co-localize with KAP1, in contrast to non-repressive KRAB domains (FIG. 2).

Interestingly, repressive KRAB domains were mostly found in proteins with the simplest domain architecture consisting of just a KRAB domain and a zinc-finger array, while the non-repressive KRAB domains were mostly found in genes that also include a DUF3669 or SCAN domain (FIG. 2). In fact, only one KRAB in a DUF3669-containing gene, ZNF783, was a repressor. ZNF783 is an uncharacterized DUF3669-KRAB-containing gene that uniquely lacks a zinc finger array (despite its name), suggesting it is distinctive among this class of transcription factors in both its effector function and its mode of localizing to targets.

The compound domain architecture that included a SCAN or DUF3669 is more common in evolutionary old KRAB genes (Imbeault et al., 2017). Here, a clear relationship was observed between the evolutionary age of the KRAB genes and the KRAB repressor strength, with KRAB domains from genes pre-dating the marsupial-human common ancestor having no repressor activity, and KRAB domains from genes that evolved later consistently functioning as strong repressors (FIG. 2). Together, these results support a model of an ancient generation of non-repressor KRAB genes followed by a more recent massive expansion of repressor KRAB genes that recruit KAP1 to silence genomic targets.

### Example 5

Deep Mutational Scan of the CRISPRi ZNF10 KRAB Effector Identifies Mutations that Modulate Gene Silencing

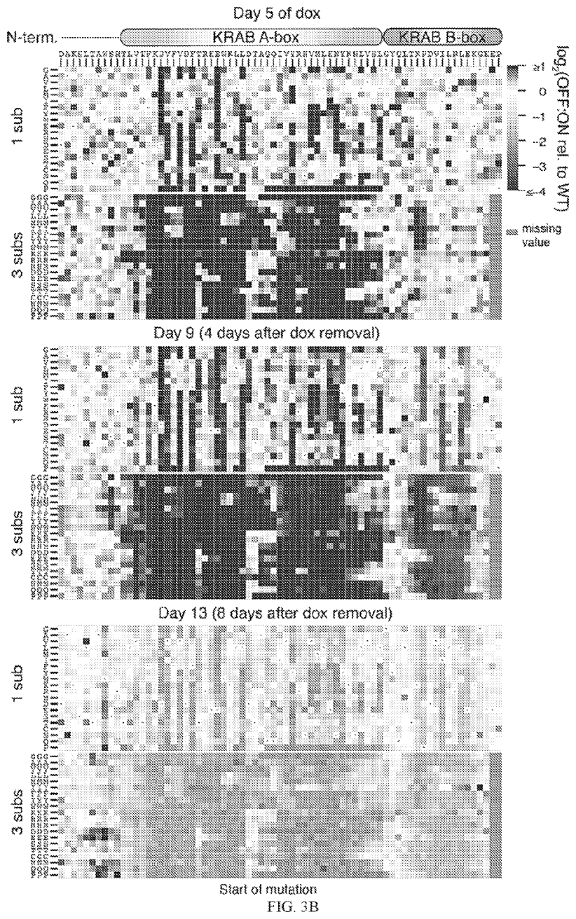

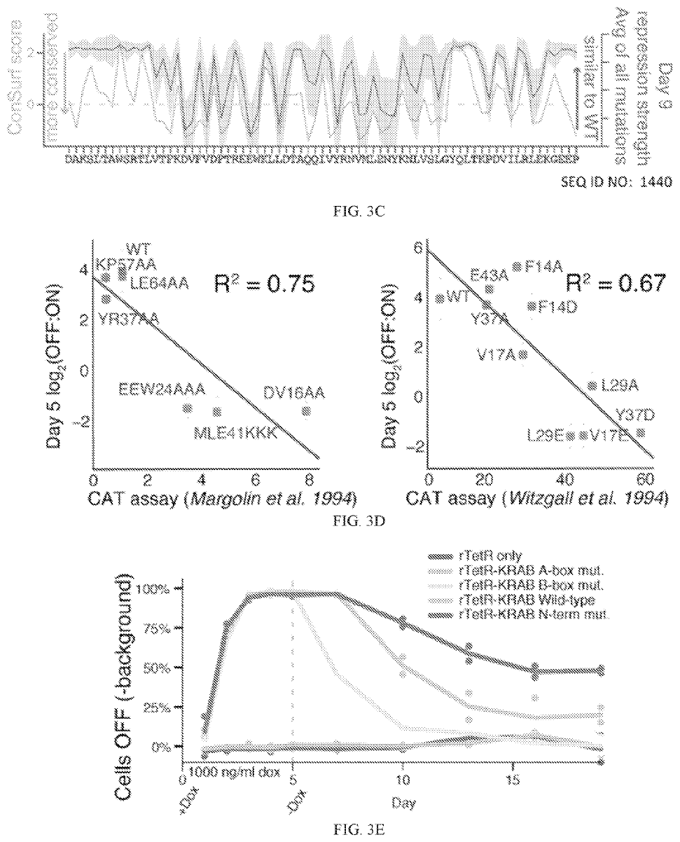

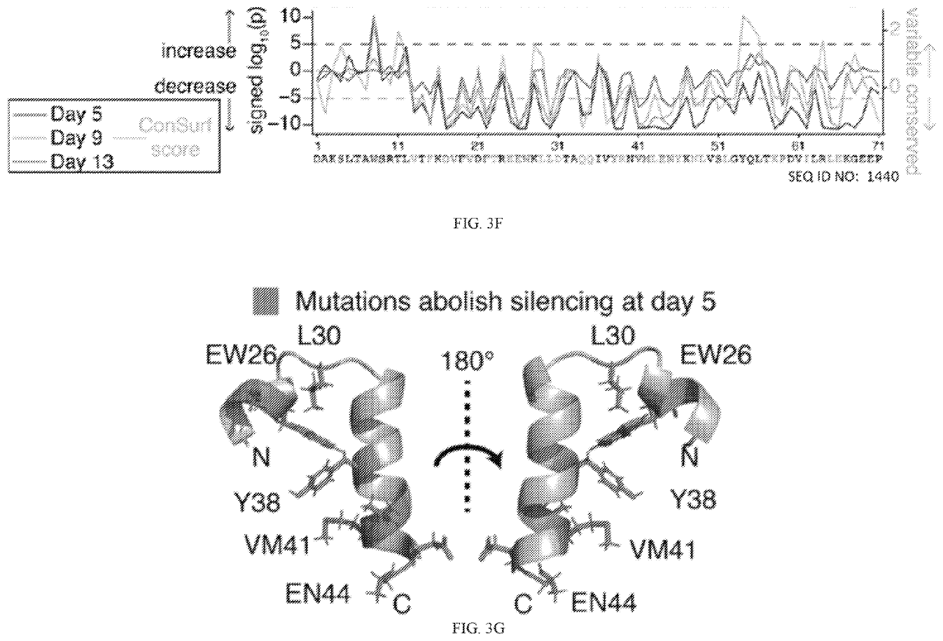

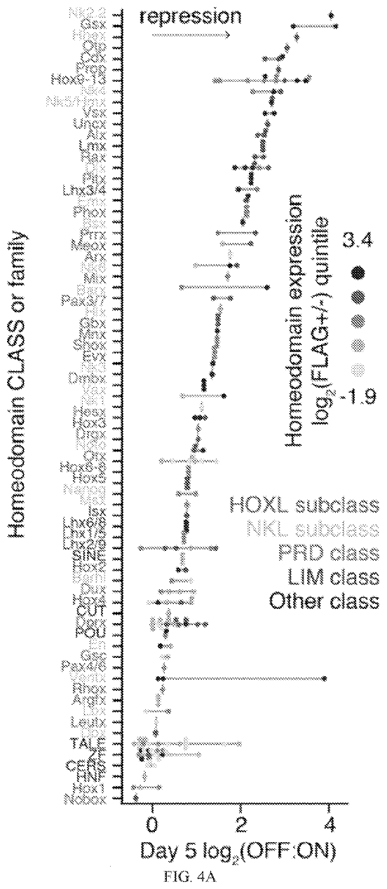

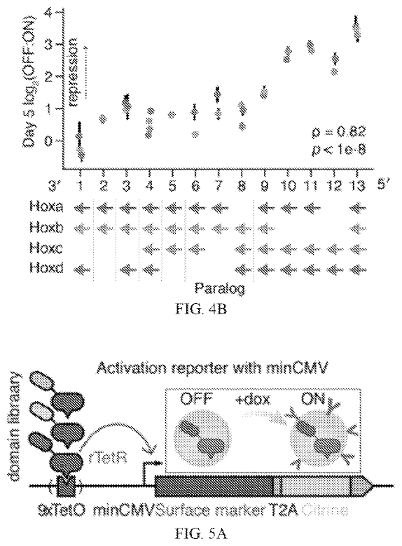

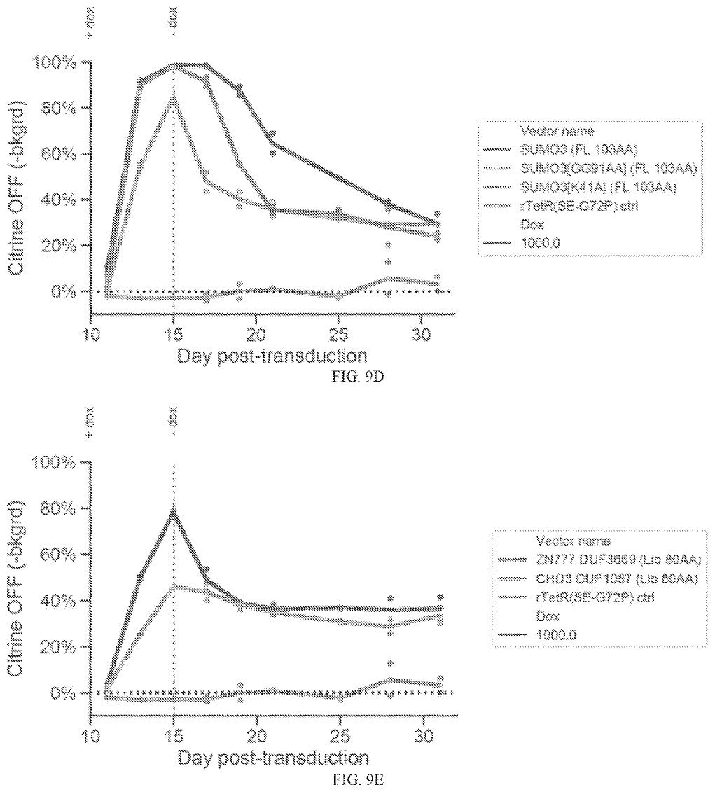

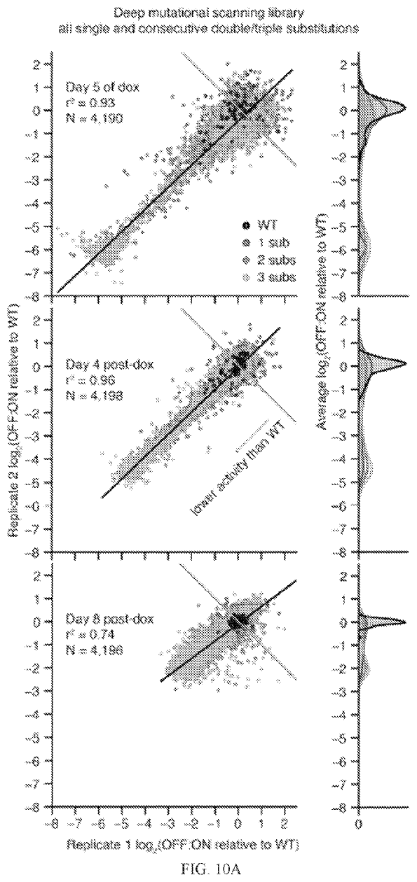

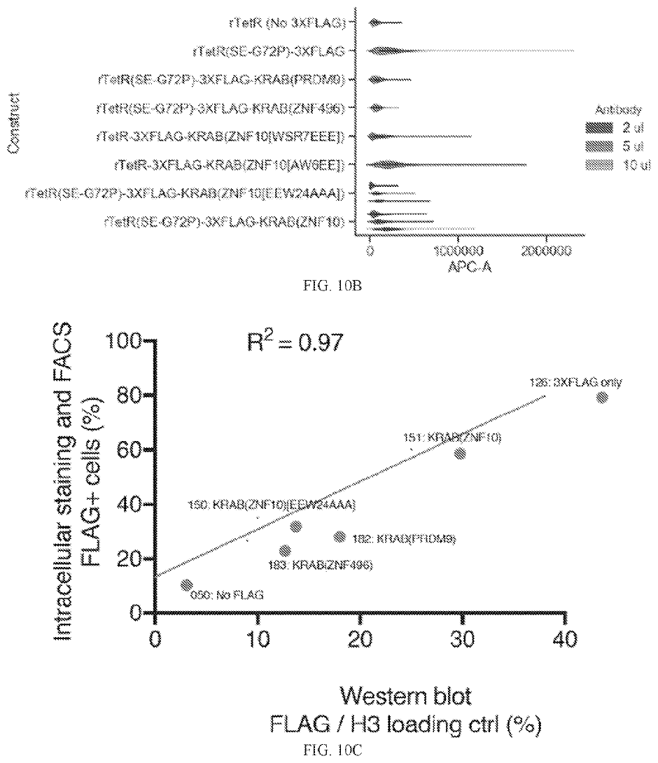

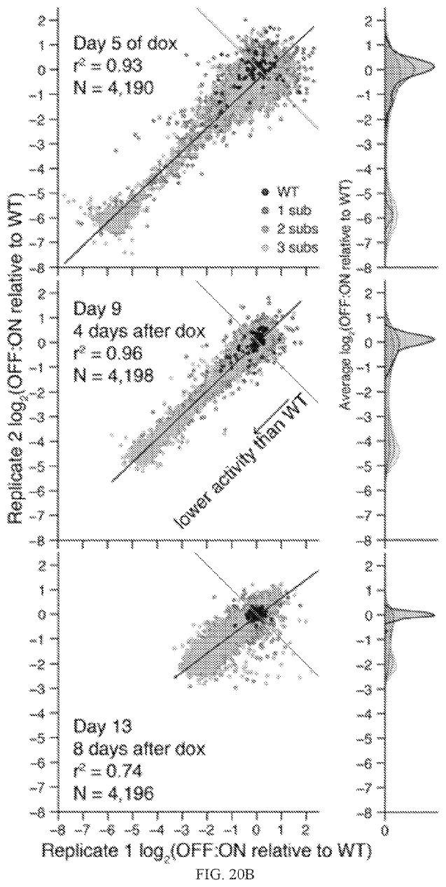

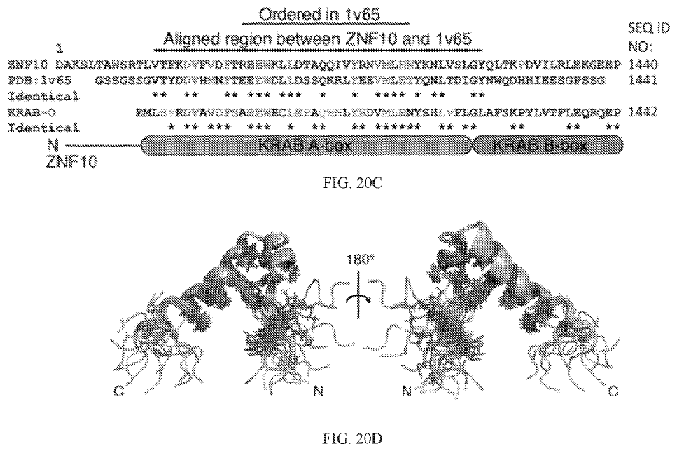

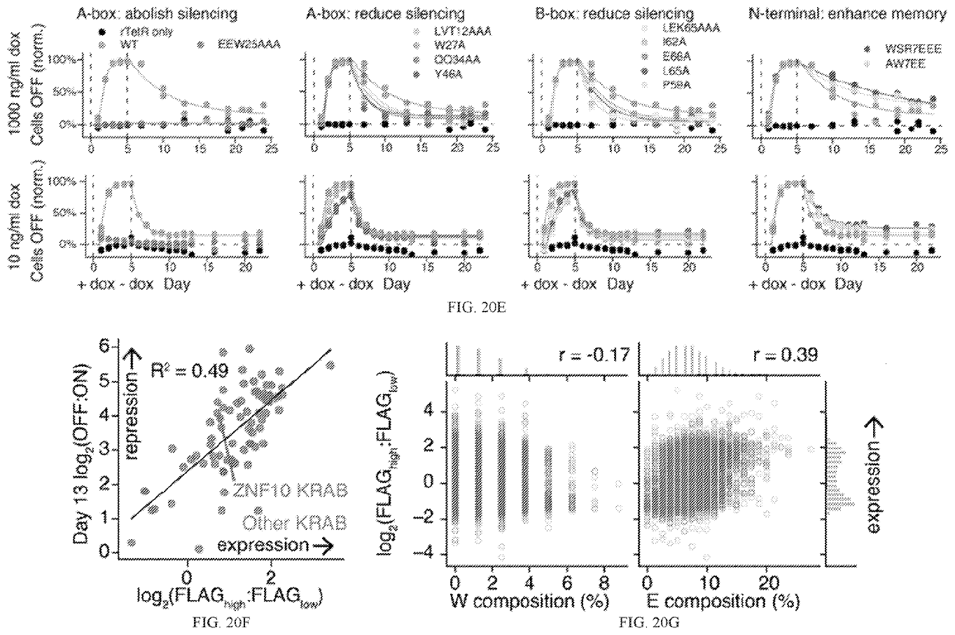

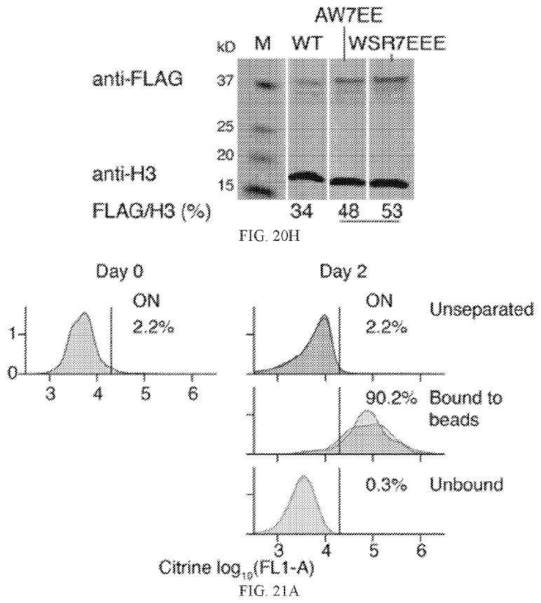

The KRAB domain from ZNF10 has been extensively used in synthetic biology applications for gene repression and is fused to dCas9 in the programmable epigenetic and transcriptional control tool known as CRISPR interference (Gilbert et al., 2014). To better understand its sequence-function relationships, a deep mutational scan (DMS) of this KRAB was performed domain using HT-recruit. A library with all possible single substitutions and all consecutive double and triple substitutions was designed (FIG. 3). To improve the ability to unambiguously align sequencing reads, variable codon usage was used to implement silent barcodes in the domain coding sequence such that the DNA sequences were more unique than the amino acid sequences (FIG. 3). HT-recruit was performed using the reporter and workflow in FIG. 1: 5 days of doxycycline induction and magnetic separation of ON and OFF cells at days 5, 9, and 13 (FIGS. 20A and 10). These measurements were highly reproducible and showed a general trend of increasing deleteriousness with increasing mutation length from singles to triples, as expected (FIGS. 20B and 10). Further, these results were compared with the KRAB amino acid conservation and a striking correlation was found between conservation and deleteriousness of mutations (FIG. 3). Amino acid and nucleic acid sequences for KRAB repressor mutants identified are shown in Table 3. Each repressor mutant score is shown relative to 0 for the wild-type sequence, with higher scores representing more enhanced KRAB transcriptional repression.

The ZNF10 KRAB effector has 3 components: the A-box which is necessary for binding KAP1 (Peng et al., 2009), the B-box which is thought to potentiate KAP1 binding (Peng et al., 2007), and an N-terminal extension that is natively found on a separate exon upstream of the KRAB domain (FIG. 3). Mutations at numerous positions in the A-box dramatically lowered repressor activity relative to the wildtype sequence (FIG. 3). Several of these mutations had previously been tested with a recruitment CAT assay in COS and 3T3 cells; those data correlated well with measurements from the deep mutational scan in K562 cells (FIG. 3). The complete lack of silencing function in an A-box KRAB mutant was also individually validated (FIG. 3). The mutational impacts across the Abox appeared to be periodic, suggesting the angle of these residues along an alpha helix could be functionally relevant (FIG. 3). These residues were designated as necessary for silencing (p<1e-5, Wilcoxon rank sum test comparing distribution of all substitutions against wild-type at day 5) and 12 necessary residues with strong mutational impacts in the A-box and one residue with significant but weak effects in the B-box were found (FIG. 3).

These substitutions were mapped onto an aligned mouse KRAB A-box structure (PDB: 1v65, 55% identity, 69% similarity in A-box [V13-Y54], FIGS. 20C and 10) and the necessary residues were found to be similarly oriented in 3D space, suggestive of a binding interface (FIGS. 3 and 20D, red, and 10). These residues may be important for KAP1 binding as 10 out of 12 of these A-box residues were in fact shown to facilitate KAP1 binding in a previous recombinant protein binding assay (Peng et al., 2009) using KRAB-O, which aligns to ZNF10 KRAB 12-71 (50% identity, 75% similarity) in a region containing all 12 of the necessary residues (red KRAB-O residues, FIGS. 20C and 10). The remaining 8 out of 8 residues previously found unnecessary for binding were also not necessary for repression in the DMS (p<1e-4, Fisher's exact test, grey KRAB-O residues, FIGS. 20C and 10). The DMS day 5 silencing scores were inspected for the individual single, double, and triple alanine substitutions used in the binding assay, and perfect agreement was found: mutations that ablated binding also abolished silencing (Z-score<−4 compared to wild-type distribution), and mutations that did not affect binding also did not affect silencing (|Z-score|<0.6) (p<0.01, n=12 mutations, Fisher's exact test). This high validation rate, and their positioning in the 3D structure, suggests the remaining 2 out of 12 necessary A-box residues from the DMS (V41 and N45) may also be involved in KAP1 binding.

In contrast to the A-box, B-box mutations showed relatively little effect at the end of recruitment (day 5), with only one statistically significant position (P59) showing consistent but weak effects. Meanwhile P59 and 4 other positions (K58, I62, L65, E66) showed a significant effect on memory after doxycycline removal as measured at day 9 (FIG. 3). Individual validations were performed for 4 significant positions and, as in the high-throughput experiment, the B-box mutants were strong gene silencers after day 5 of recruitment but showed reduced memory after doxycycline release (FIGS. 3 and 20E and 10). To interpret this result, the previously proposed gene silencing model in which silenced cells pass through a ‘reversibly silent’ state before entering an ‘irreversibly silent’ state was considered (Bintu et al., 2016). The B-box mutant memory reduction may be the result of a moderate silencing speed reduction, resulting in fewer cells committing to the irreversibly silent state by day 5, and that the mutational impact on silencing speed was masked because reversibly silent and irreversibly silent cells are indistinguishable at day 5. To test this possibility, the silencing time course was repeated with a 100-fold lower dose of doxycycline in order to tune down the recruitment strength. In this regime, the B-box mutations reduced silencing speed before day 5 (FIGS. 20E and 10). This result shows the B-box has a partial contribution to KRAB silencing speed.

Lastly, the KRAB N-terminus contained residues where many substitutions consistently enhanced silencing relative to wild-type (FIGS. 3, blue, day 13 panels). In particular, nearly all substitutions for the tryptophan at position 8 led to higher numbers of cells silenced relative to wild-type at day 13 (which is the time point with the most dynamic range to detect silencing levels above wild-type). This was the only significant position for enhanced silencing (FIG. 3). The memory enhancement for two of the highest-ranked of these mutants (WSRSEEE and AW7EE) was individually validated with high-doxycycline recruitment (FIGS. 3 and 20E and 10).

This silencing enhancement may have been a result of enhanced KRAB protein expression level. To investigate the relationship between protein expression level and KRAB silencing strength, the high-throughput FLAG-tag expression level measurements for the set of KAP1-binding KRAB domains was inspected and a significant correlation was found between KRAB expression level and silencing at day 13 (r²=0.49, FIGS. 20F and 10). Most relevant to the deep mutational scan results, ZNF10 KRAB had lower expression levels compared to other KRAB domains that showed higher day 13 silencing levels, implying that it could be improved via mutations. Notably, the N-terminus was very poorly conserved (FIG. 3) and was in fact uniquely found in the KRAB from ZNF10 by BLAST, suggesting that stability-improving mutations in the N-terminus would be unlikely to interfere with KRAB function. In addition, across the entire domain expression higher tryptophan (W) frequency was observed in a domain that was negatively correlated with expression level while higher glutamic acid (E) frequency was positively correlated with expression level (FIGS. 20G and 10). This amino acid composition trend further suggested that the N-terminal KRAB mutant enhancements could be due to improved expression level, as substituting out the tryptophan from KRAB position 8 enhanced its effector function and that this enhancement was most pronounced when substituting with glutamic acid. A Western blot for the ZNF10 KRAB variants confirmed that the N-terminal glutamic acid substitution mutants were more highly expressed than the wild-type (FIGS. 20H and 10). Together, these results demonstrated the use of a deep mutational scan both to map sequence-to-function for a human transcriptional repressor and to improve effectors by incorporating expression-enhancing substitutions into poorly conserved positions.

### Example 6

Homeodomain Repressor Strength is Colinear with Hox Gene Organization

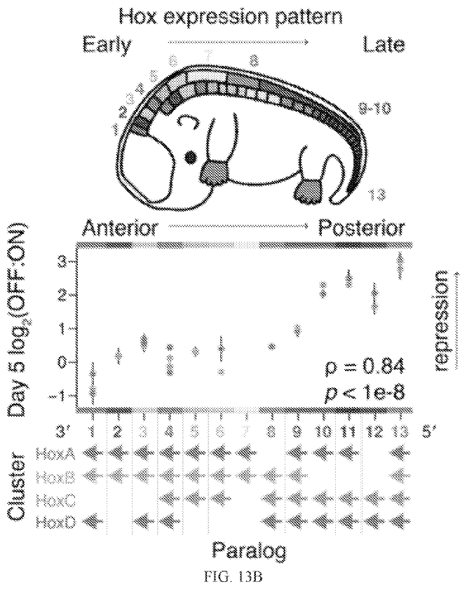

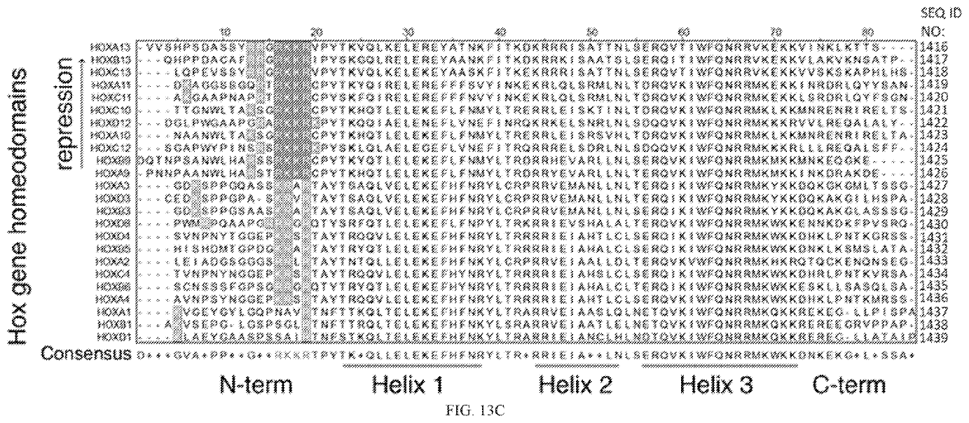

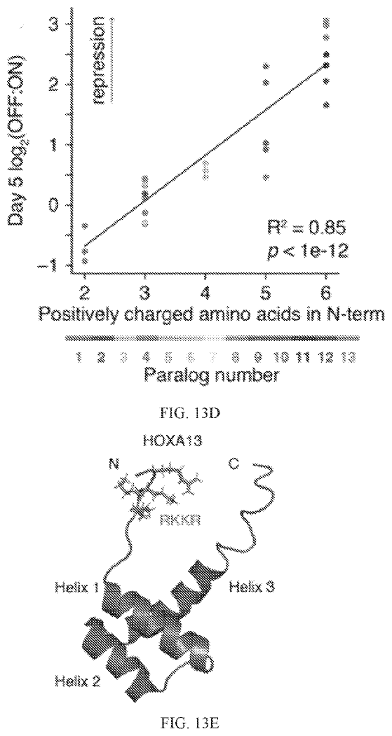

The second largest domain family that included repressor hits in the screen was the homeodomain family. Homeodomains are composed of 3 helices and are sequence-specific DNA binding domains that make base contacts through Helix 3 (Lynch et al., 2006). In some cases, they are also known to act as repressors (Holland et al., 2007; Schnabel and Abate-Shen, 1996). The library included the homeodomains from 216 human genes, and 26% were repressor hits. The repressors were found in 4 out of the 11 subclasses of homeodomains: PRD, NKL, HOXL, and LIM (FIG. 13A). These recruitment assay results suggested that transcriptional repression could be a widespread, though not ubiquitous, function of homeodomain transcription factors.

Next, the HOXL subclass results were inspected more closely. This subclass contained the Hox genes, a subset of 39 homeodomain transcription factors that are master regulators of cell fate and specify regions of the body plan along the anterior-posterior axis during embryogenesis. These genes are found in four Hox paralog clusters (A to D) arranged co-linearly from 3′ to 5′ corresponding to the temporal order and spatial patterning of their expression along the anterior-posterior axis (Gilbert, 1971). Interestingly, the repressor strength of their homeodomains was also collinear with their arrangement in the Hox clusters, such that the more 5′ gene homeodomains were stronger repressors (Spearman's p=0.82, FIG. 13B). This correlation suggested a possible link between homeodomain repressor function and Hox gene expression timing and anterior-posterior axis spatial patterning.

Multiple sequence alignment of the Hox homeodomains revealed an RKKR (SEQ ID NO: 1330) motif present in the N-terminal arm of the 11 strongest repressor domains (FIG. 13C). The motif resided in a basic context in the strongest repressors, while the lower ranked domains lack the motif but still contained some basic residues in the disordered N-terminal arm, resulting in a significant correlation between repression strength and the number of positively charged amino acids arginine and lysine (R²=0.85, FIGS. 13C-13E).

Outside the Hox homeodomains, 99.5% of the repressor hits in the Pfam nuclear protein domain library did not contain the RKKR (SEQ ID NO: 1330) motif, while many non-hits did. Also, there was no correlation between net domain charge and repression strength at day 5 when considering the full library of domains (R²=0.04). Together, these results suggested the RKKR (SEQ ID NO: 1330) motif and charge contributed to Hox homeodomain repression in the recruitment assay, but they were not sufficient for repression when found in the context of other domains.

### Example 7

### Discovery of Transcriptional Activators by HT-Recruit to a Minimal Promoter

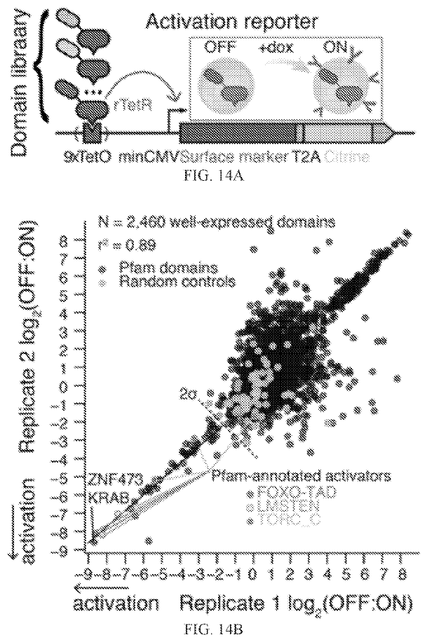

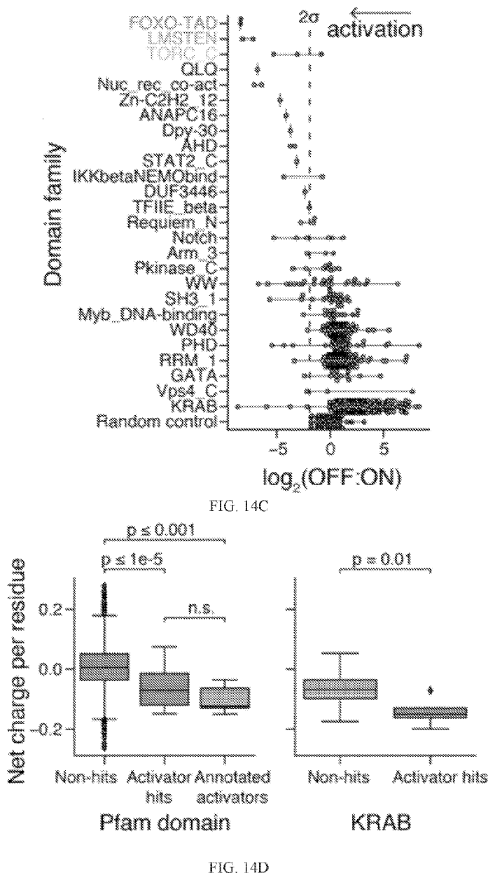

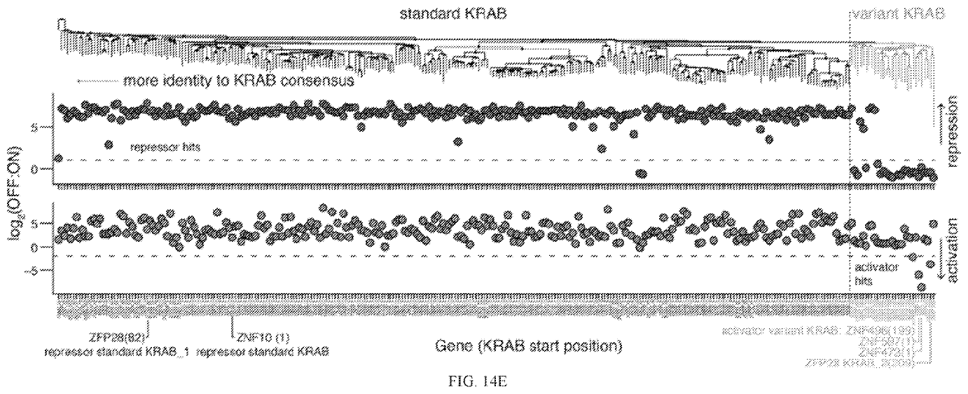

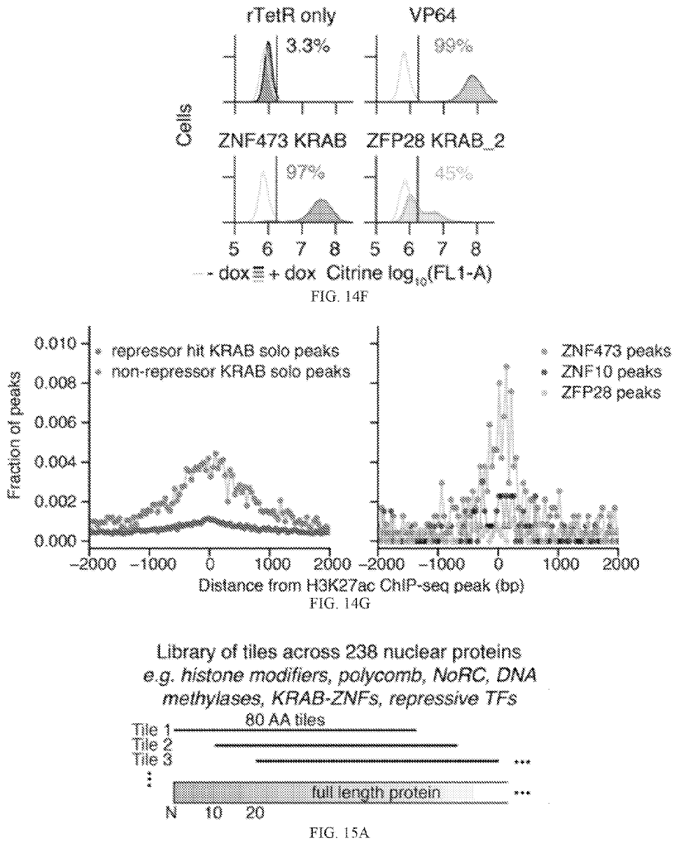

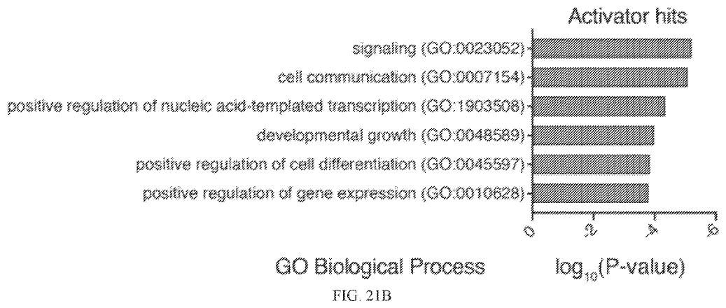

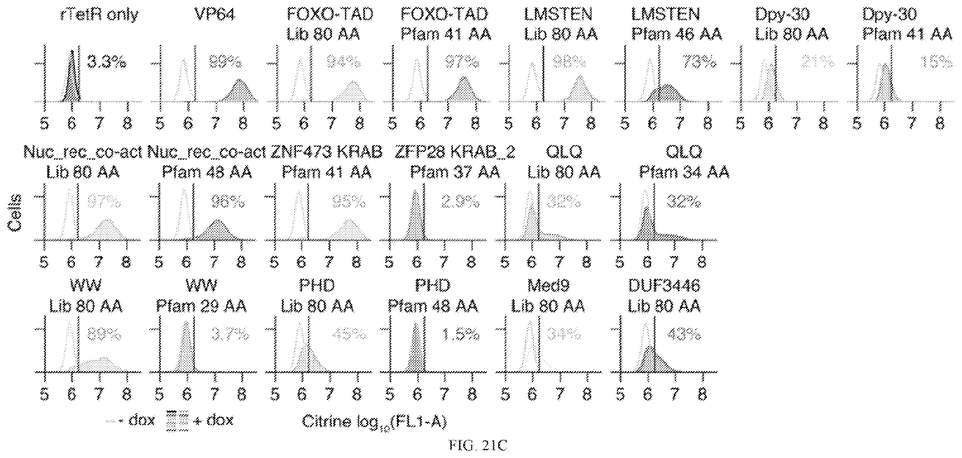

It was established that a reporter K562 line with a weak minimal CMV (min CMV) promoter that could be activated upon recruitment of fusions between rTetR and activation domains (FIG. 14A). To perform the activator screen, lentivirus was used to deliver the nuclear Pfam domain library to these reporter cells, rTetR-mediated recruitment was induced with doxycycline for 48 hours, the cells (FIG. 21A) were magnetically separated and the domains in the two resulting cell populations were sequenced. An enrichment ratio from the sequencing counts in the bead-bound (ON) and unbound (OFF) populations for each domain as a measure of transcriptional activation strength was computed and hits were two standard deviations past the mean of the poorly expressed negative controls (FIG. 14B). The hits included three previously known transcriptional activation domain families that were present in the library: FOXO-TAD from FOXO1/3/6, LMSTEN from Myb/Myb-A, and TORC_C from CRTC1/2/3. Activation strength measurements for the hits were highly reproducible between separately transduced biological replicates (r²=0.89, FIG. 14B). This second screen with the short nuclear domain library established that HT-recruit can be used to measure either activation or repression by changing the reporter's promoter. Amino acid and nucleic acid sequences for activators identified in the nuclear Pfam domain library are shown in Table 2, with lower scores indicated stronger activators.

In total, 48 hits from 26 domain families were found. Beyond the three known activator domain families above, the remaining families with an activator hit were not previously annotated on Pfam as activator domains (FIG. 14C). Overall, fewer activators were found than repressors, which may simply be because activators are often disordered or low-complexity regions (Liu et al., 2006) that are frequently not annotated as Pfam domains. However, the proteins containing activator domains were significantly enriched for gene ontology terms such as ‘positive regulation of transcription’ and the strongest enrichment was for ‘signaling’, which reflects that many of their source proteins are activating factors (FIG. 21B). Further, the hits were significantly more acidic than non-hits (p≤1e-5, Mann Whitney test, FIG. 14D), a common property in activation domains (Mitchell and Tjian, 1989; Staller et al., 2018).

Several hits were not sourced from sequence-specific transcription factors where classical activator domains are expected but were instead nonclassical activators from co-activator and transcriptional machinery proteins including Med9, TFIIEβ, and NCOA3. In particular, the Med9 domain, whose ortholog directly binds other mediator complex components in yeast (Takahashi et al., 2009), was a strong activator with an average log 2(OFF:ON)=−5.5, despite its weak expression level. Nonclassical activators have previously been reported to work individually in yeast (Gaudreau et al., 1999) but only weakly when individually recruited in mammalian cells (Nevado et al., 1999). One exception is TATA-binding protein (Dorris and Struhl, 2000). By screening more nonclassical sequences, more exceptions to this notion were found.

For all tested domains, doxycycline-dependent activation of the reporter gene was confirmed using both the extended 80 AA sequence from the library and the trimmed Pfam-annotated domain (FIG. 21C). The previously annotated FOXO-TAD and LMSTEN were strong activators, in both their extended and trimmed versions. The activator function of DUF3446 from the transcription factor EGR3 and the largely uncharacterized QLQ domain from the SWI/SNF family SMARCA2 protein was also confirmed. Further, it was confirmed that the Dpy-30 motif domain, a DUF found in the Dpy-30 protein, was a weak activator. Dpy-30 was a core subunit of histone methyltransferase complexes that write H3K4me3 (Hyun et al., 2017), a chromatin mark associated with transcriptionally active chromatin regions (Sims et al., 2003). In total, 11 hit domains (including nonclassical hits Med9 and Nuc_rec_co-act from NCOA3) were tested and all were found to significantly activate the reporter, when using the extended 80 AA sequence from the library. Together, the screen and validations demonstrated that the unbiased nuclear protein domain library can be productively re-screened to uncover domains with distinct functions, and that a diverse set of domains beyond classical activation domains (and including DUFs) can activate transcription upon recruitment.

### Example 8

### Discovery of KRAB Activator Domains

Surprisingly, the strongest activator in the library was the KRAB domain from ZNF473 (FIG. 5B). Three other KRAB domains (from ZFP28, ZNF496, and ZNF597) were also activator hits, all of which were stably expressed and not repressors. One of these domains, from ZNF496, had previously been reported as an activator when recruited individually in HT1080 cells (Losson and Nielsen, 2010). Interestingly, ZFP28 contains two KRAB domains; KRAB_1 was a repressor and KRAB_2 was an activator. Previous affinity-purification/mass-spectrometry performed on full-length ZFP28 identified significant interactions with both repressor and activator proteins (Schmitges et al., 2016). The activator KRAB domains were significantly more acidic than nonactivator KRABs (p=0.01, Mann Whitney test, FIG. 14D). Sequence analysis showed they were divergent from the consensus KRAB sequence while sharing homology to each other and formed a variant KRAB subcluster (FIG. 14E). Previous phylogenetic analysis has linked the variant KRAB cluster to a lack of KAP1 binding and older evolutionary age (Helleboid et al., 2019). More specifically, two of the activator KRAB source proteins (ZNF496 and ZNF597) had previously been tested with co-immunoprecipitation mass-spectrometry and were not found to interact with KAP1 (Helleboid et al., 2019).

It was individually validated that the KRAB from ZNF473 as a strong activator and KRAB_2 from ZFP28 as a moderate strength activator (FIG. 14F), using the same 80 AA sequence centered on the KRAB domains that was used in the library. Further, the trimmed 41 AA KRAB from ZNF473 was sufficient for strong activation, while the trimmed 37 AA KRAB_2 from ZFP28 did not activate, implying some of the surrounding sequence was required for activation (FIG. 21C). Next, available ChiP-seq and ChIP-exo datasets were inspected (ENCODE Project Consortium et al., 2020; Imbeault et al., 2017; Najafabadi et al., 2015; Schmitges et al., 2016) and it was found that ZNF473 co-localized with the active chromatin mark H3K27ac, in contrast to the repressive ZNF10 (FIG. 14G). Upon manual inspection, the most significant ZNF473 peaks were found near the transcription start site of genes (CASC3, STAT6, WASF2, ZKSCAN2) and a lncRNA (LINC00431). Meanwhile, ZFP28 did not co-localize with H3K27ac, perhaps indicating its KAP1-binding repressor KRAB_1 domain was generally the dominant effector over its moderate strength activator KRAB_2 domain. Looking beyond these individual KRAB proteins, the zinc finger proteins that contain a repressor KRAB did not co-localize with H3K27ac while the non-repressive KRAB proteins as a group did include colocalized peaks (FIG. 14G). Together, the results support that variant KRAB proteins are functionally diverse, sometimes functioning as transcriptional activators.

### Example 9

### Tiling Library Uncovers Effector Domains in Unannotated Regions of Nuclear Proteins

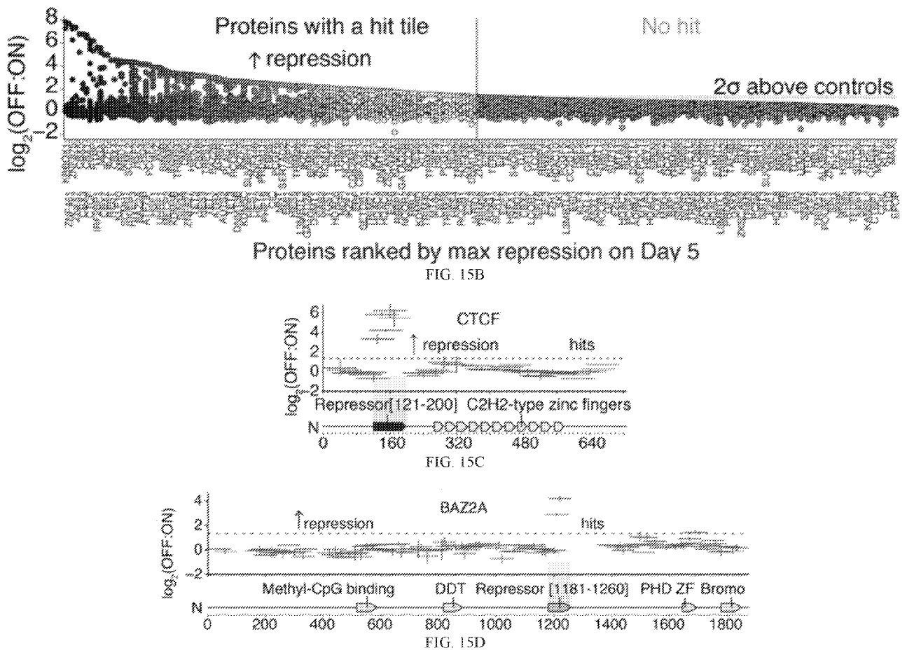

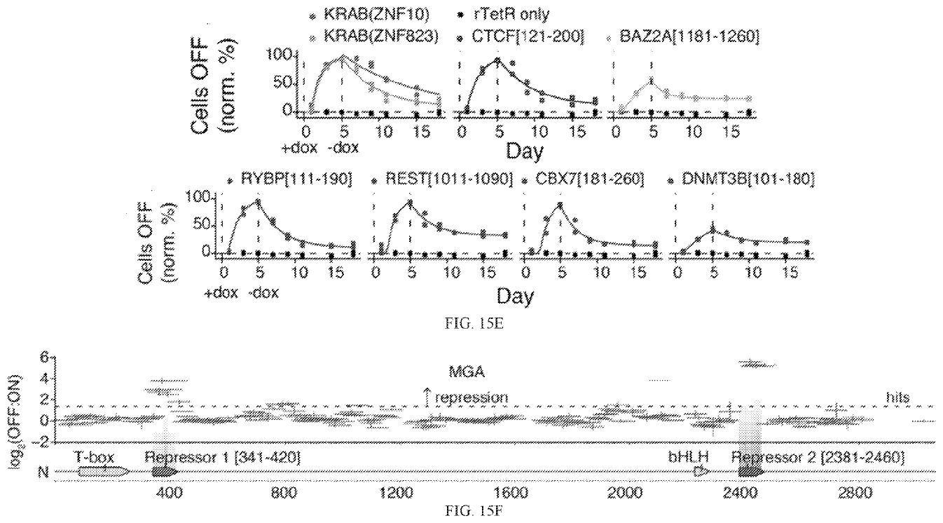

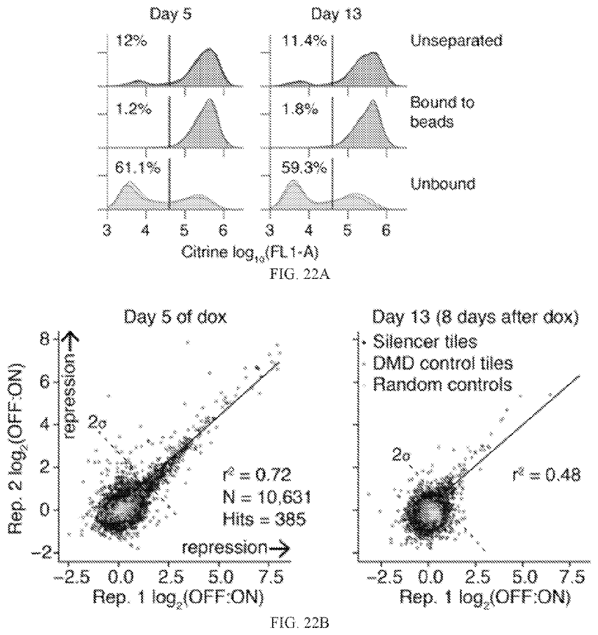

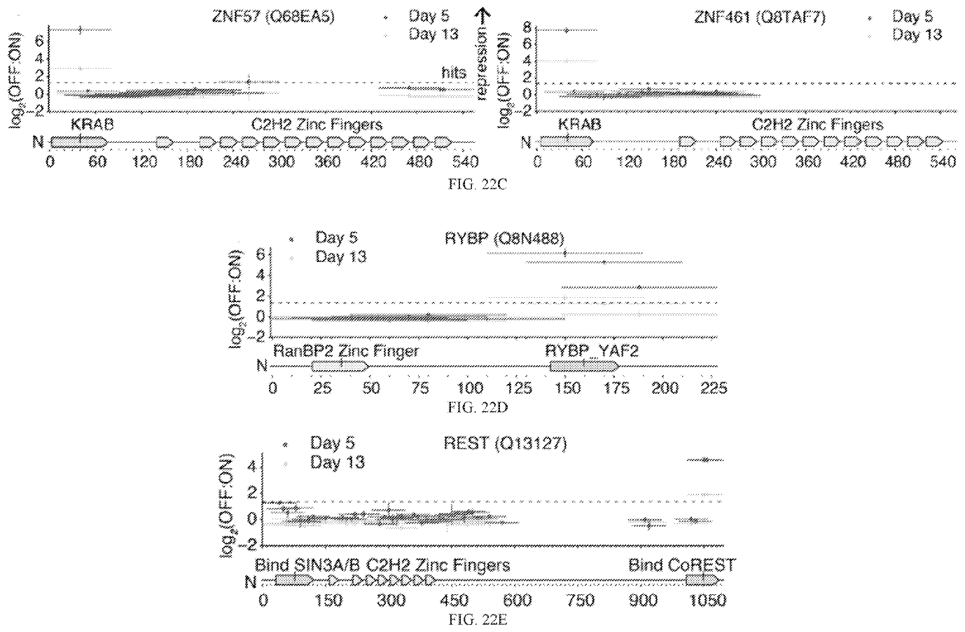

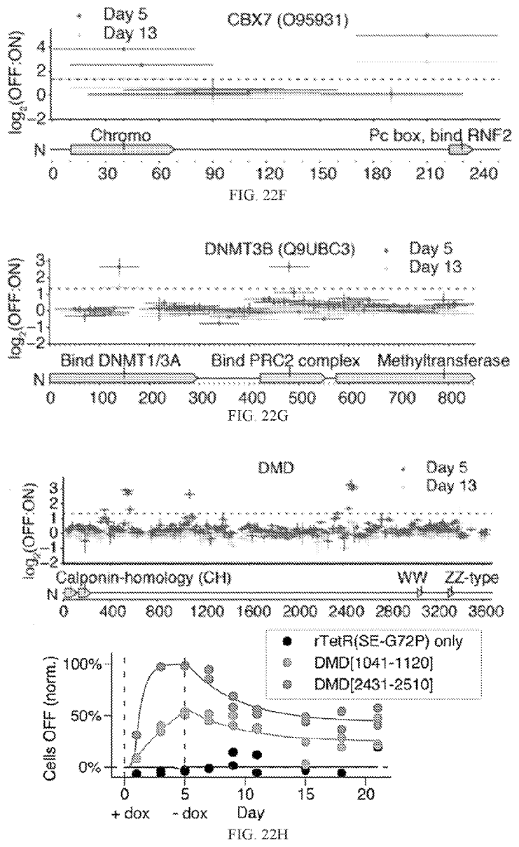

Pfam annotations provided one useful means of filtering the nuclear proteome to generate a relatively compact library, but Pfam is likely currently missing many of the human effector domains. In order to discover effector domains in unannotated regions of proteins, a tiling library was designed by curating a list of 238 proteins from silencer complexes and tiling their sequences with 80 amino acids separated by a 10 amino acid tiling window (FIG. 15A). High-throughput recruitment to the strong pEF reporter was performed and time points were taken after 5 days of doxycycline to measure silencing, and again at day 13 (8 days after doxycycline release) to measure epigenetic memory (FIG. 22A), 4.3% of the tiles scored as hits at day 5 (FIG. 15B) and their repressor strength measurements were reproducible (r²=0.72, FIG. 22B) Altogether, the tiling screen found short repressor domains in 141/238 proteins. Some of these hits include positive controls overlapping annotated domains: for example, by tiling ZNF57 and ZNF461, the KRAB domains of these transcription factors were identified as repressive effectors, and not the rest of the sequence (FIG. 22C). Similarly, the tiling strategy identified the RYBP repressive domain annotated by Pfam, and both the 80 AA tile and the 32 AA Pfam domain silenced with similar strength and epigenetic memory in individual validations (FIG. 22D). Repressors in REST (overlapping the CoREST binding domain (Ballas et al., 2001)), DNMT3b (overlapping the DNMT1 and DNMT3a binding domain (Kim et al., 2002), and CBX7 (overlapping the PcBox that recruits PRC1 (Li et al., 2010)) were also identified and validated (FIGS. 22E-22G). Another category of tiling hits was not annotated as domains in Pfam, but previous reports were found of their repressor function in the literature. For example, amino acids 121-220 of CTCF had a strong repressive function in the screen and when validated individually (FIGS. 15C and 15E), consistent with previous recruitment studies in HeLa, HEK293, and COS-7 cells (Drueppel et al., 2004). Together, these results established that high-throughput recruitment of protein tiles was an effective strategy to identify bona fide repressor domains. Amino acid sequences for repressors identified in the tiling library are shown in Table 4, with higher scores indicating increased repression.

Novel unannotated repressor domains were also discovered. For example, BAZ2A (also known as TIP5) is a nuclear remodeling complex (NoRC) component that mediates transcriptional silencing of some rDNA (Guetg et al., 2010), but does not have any annotated effector domains. The BAZ2A tiling data showed a peak of repressor function in a glutamine-rich region and it was individually validated as a moderate strength repressor (FIGS. 15D and 15E). Repressor tiles were found in unannotated regions of three TET DNA demethylases (TET1/2/3). Unexpectedly, repressor tiles were also identified in the control protein DMD, which was validated by flow cytometry (FIG. 22H).

A MGA, which is thought to repress transcription by binding the genome at E-box motifs and recruiting the non-canonical polycomb 1.6 complex (Blackledge et al., 2014; Jolma et al., 2013; Stielow et al., 2018), tiling experiment revealed two domains with repressor function, located adjacent to the two known DNA binding domains, called here Repressor 1 and Repressor 2 (FIG. 15F). These repressor domains were individually validated and distinct dynamics of silencing and degrees of memory were observed; the first domain (amino acids 341-420) featured slow silencing but strong memory, while the second domain (amino acids 2381-2460), featured rapid silencing but weak memory with fast reactivation (FIG. 15G). These appear to be the first effector domains isolated from a protein in the ncPRC1.6 silencing complex.

Next, it was attempted to identify the minimal necessary sequence for repressor function in each independent domain by examining the overlap in all tiles covering a protein region that shows repressor function and determining which contiguous sequence of amino acids is present in all the repressive tiles (FIG. 15H). Using this approach, two candidate minimized effector domains for MGA were generated: the 10 amino acid sequence MGA [381-390] and the 30 amino acid sequence MGA [2431-2460], which both overlapped conserved regions with ConSurf-predicted functional exposed residues. Individual validation experiments demonstrated that both minimized candidates can efficiently silence the reporter (FIG. 15I).

### Example 10

### HT-Recruit Quantifies Transcriptional Effector Function Across DNA-Binding Domain, Cell Type, and Target Gene Contexts

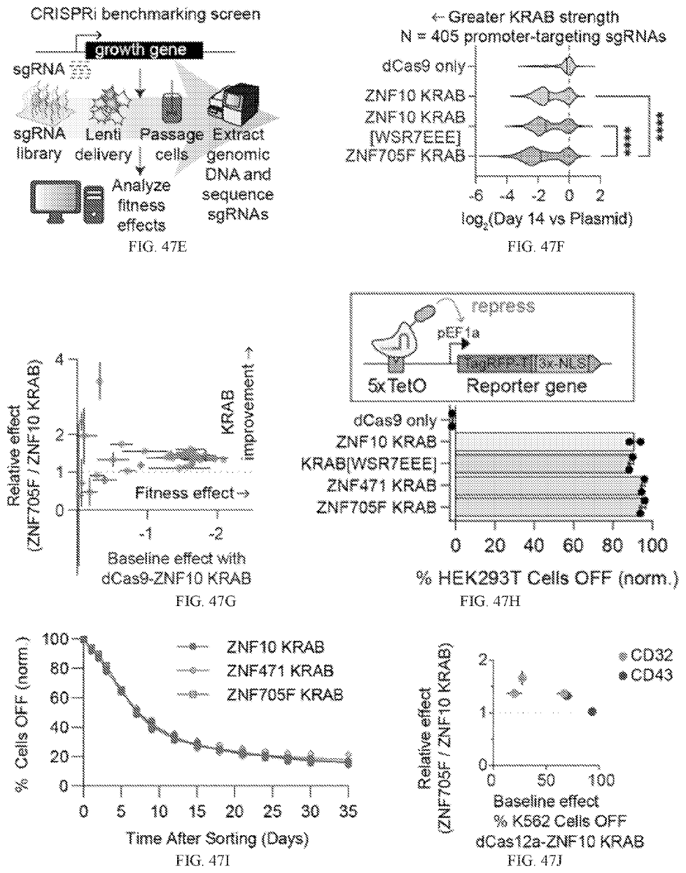

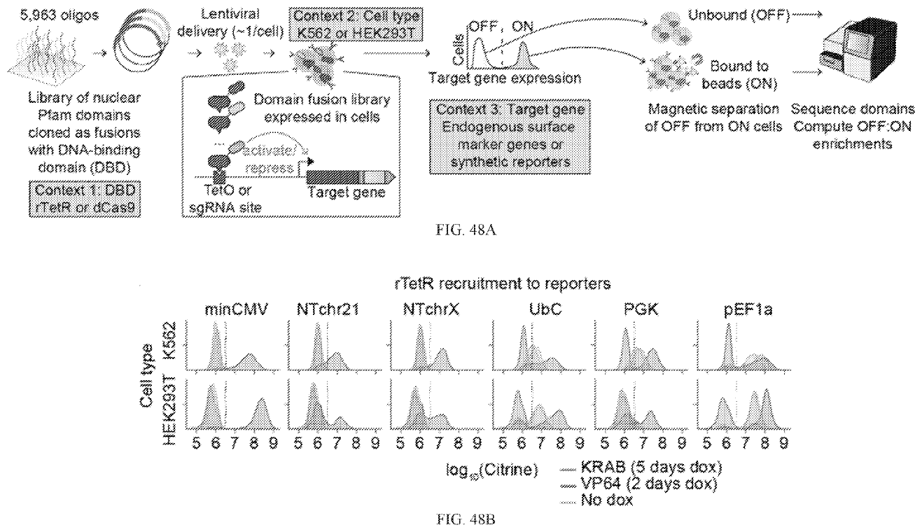

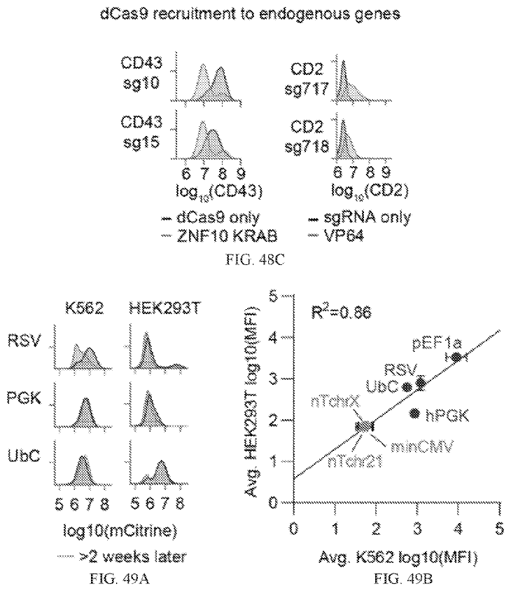

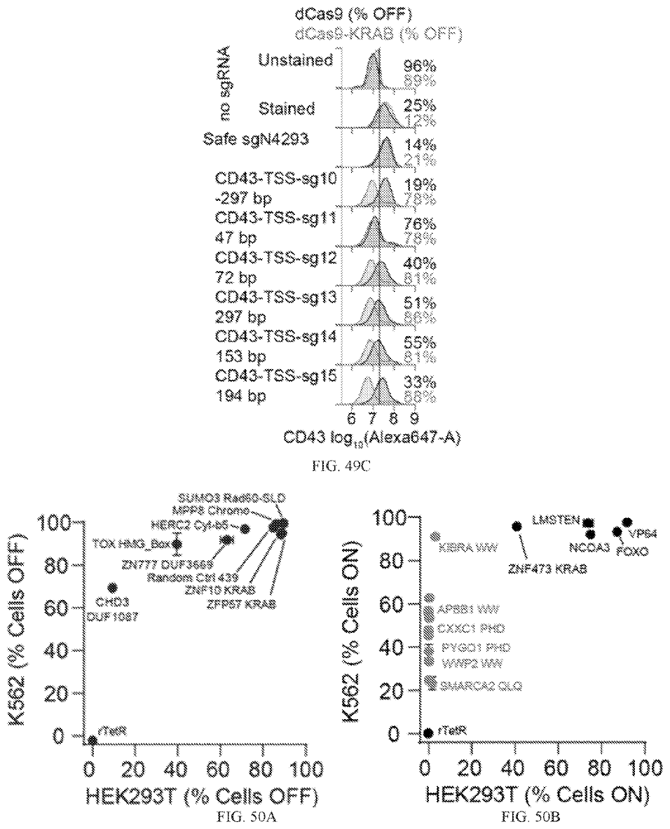

Transcriptional effector functions were measured systematically and quantitatively across biological contexts by performing high-throughput recruitment (HT-recruit) screens using two different DNA-binding domains (rTetR and dCas9) to recruit effectors to various gene targets and cell-types (FIG. 48A). In order to study different promoter contexts while keeping the rest of the scenarios the same, an array of reporters with varied promoter strength were developed. Some of these reporters contain a weak minimal promoter that is activatable upon recruitment with an activator, while others contain a strong constitutive promoter that can be silenced by a repressor (FIG. 48B). To study how effector-promoter interactions differ across cell types, these sets of reporters were installed at the AAVS1 safe harbor region using TALEN-mediated homology directed repair (HDR) in both K562 and HEK293T cells (FIG. 48A). As expected, all minimal promoters (minCMV, nonTATAchrX, and nonTATAchr21) started OFF, and could be activated with the positive activation control, VP64 (FIG. 48B; red line). Two of the constitutive promoters, pEF1a and UbC, started ON and could be repressed by the positive control, the KRAB domain from ZNF10 (FIG. 48B; blue line). The RSV promoter was rapidly silenced upon installation in both cell types (FIG. 49A), so it was not used for screens. Promoter silencing can be cell-type specific: the PGK promoter was constitutively ON in K562 cells, but was background silenced in HEK293T cells (FIG. 49A). Based on this observation, PGK was not used as a repressible promoter in K562 or as an activatable promoter in HEK293T cells (FIG. 48B), to study if a silenced full-length promoter responds differently to activator recruitment than the minimal promoters. Overall, the expression levels for each promoter were similar across the two cell types (R²=0.86) except for the PGK promoter (FIG. 49B).

In order to extend the approach to recruiting libraries of effectors to endogenous targets, the library was fused onto dCas9 and targeted endogenous surface marker genes. Targeting surface markers allowed use or fluorescent antibodies to immunostain cells, thus providing a way to monitor single-cell gene expression variability during individual recruitment assays by flow cytometry and to magnetically separate ON and OFF cells with ProG beads during HT-recruit (FIG. 48A). For repressors, the highly expressed surface marker CD43 was targeted in K562 cells. Both dCas9 alone and dCas9-KRAB from ZNF10 were recruited with several sgRNAs targeting the CD43 transcriptional start site (TSS) and two sgRNAs, sg10 and sg15, for which repression depended on the KRAB repressor domain were identified (FIGS. 48C and 49C). For activators, the lowly-expressed surface marker CD2 was targeted. Similarly, activator-dependent effects were found when testing both dCas9 and dCas9-VP64 with several sgRNAs targeting this gene (FIG. 49C; right). Further, it was confirmed that staining and magnetic separation is effective with these surface markers.

Using these components, new HT-recruit and FLAG-based expression screens were performed and combined with the previous screens across 10 rTetR-targeted synthetic reporter contexts in two cell types and 5 dCas9-targeted screens, in total charting the effector context-specificity landscape for over 5000 human nuclear protein domains across 15 contexts (FIG. 48C). To identify domains that activated and repressed in each context, the results were filtered for domains that were well-expressed. Expression was measured by permeabilizing the cells, staining with an anti-FLAG antibody, sorting into high and low protein expression level bins that account for transcription variability by using the fluorescent delivery marker which is found on the same transcript after the T2A signal, and then sequencing the domains in those binned cell populations. Domains were similarly well-expressed between cell types (R²=0.65) and whether they were fused to rTetR or dCas9 (FIG. 36, right). The results were then filtered for sufficient sequencing depth and reproducible results between biological replicates were identified. Repressors and activators with consistent function across contexts were identified. Moreover, context-dependent effectors were found.

### Example 11

### Repressor and Activator Function in Various Cell Types

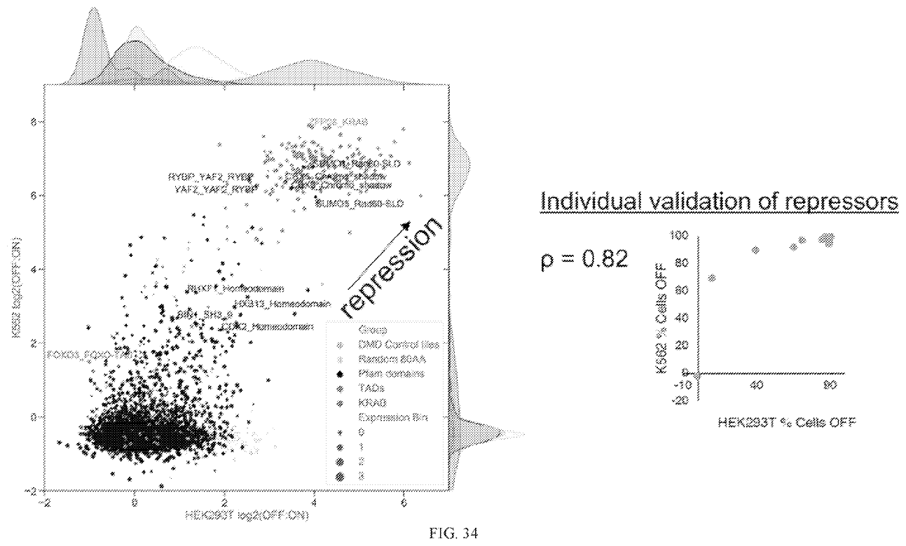

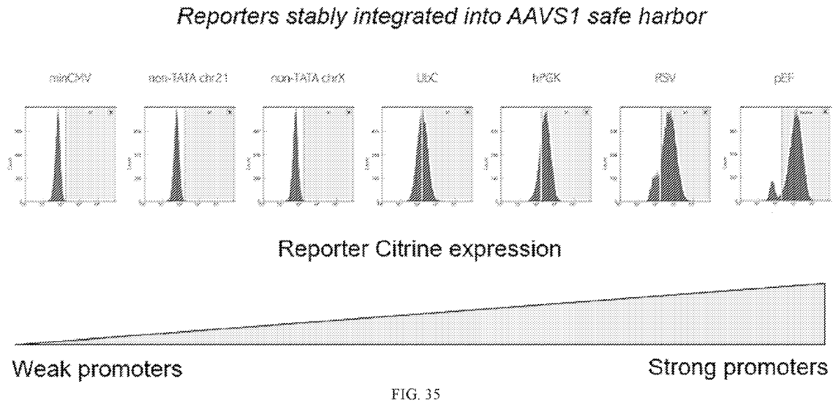

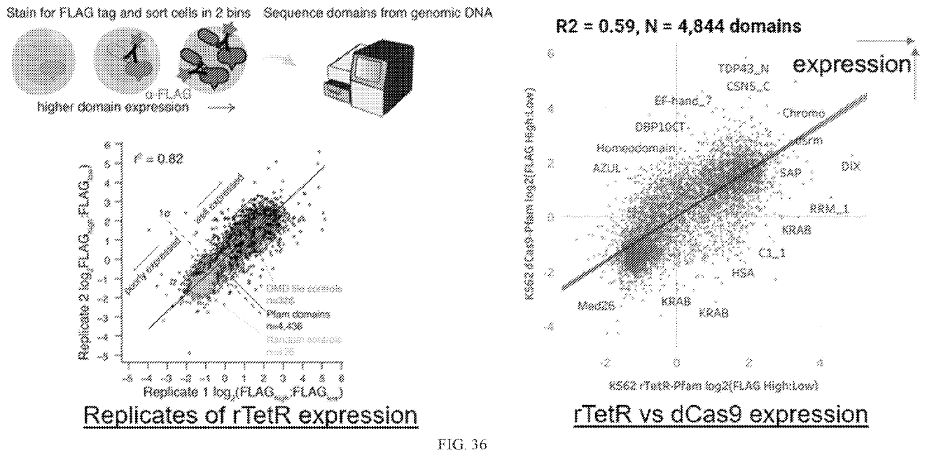

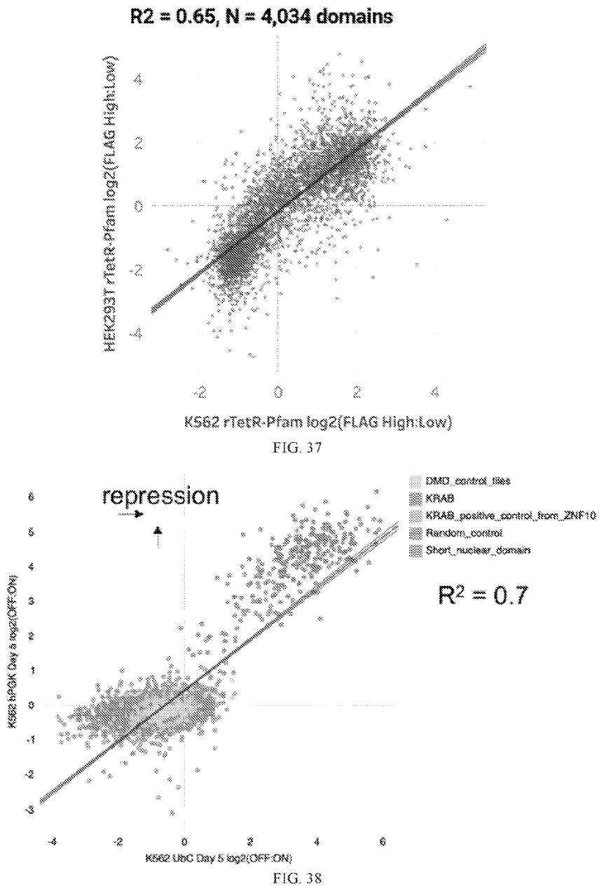

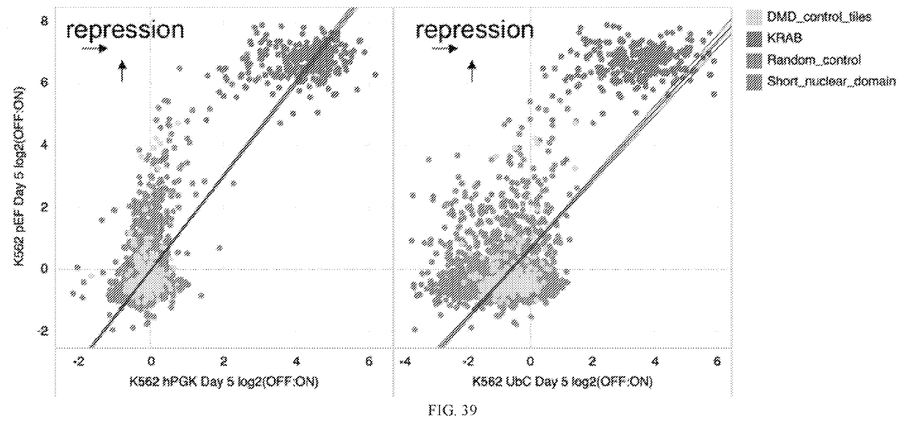

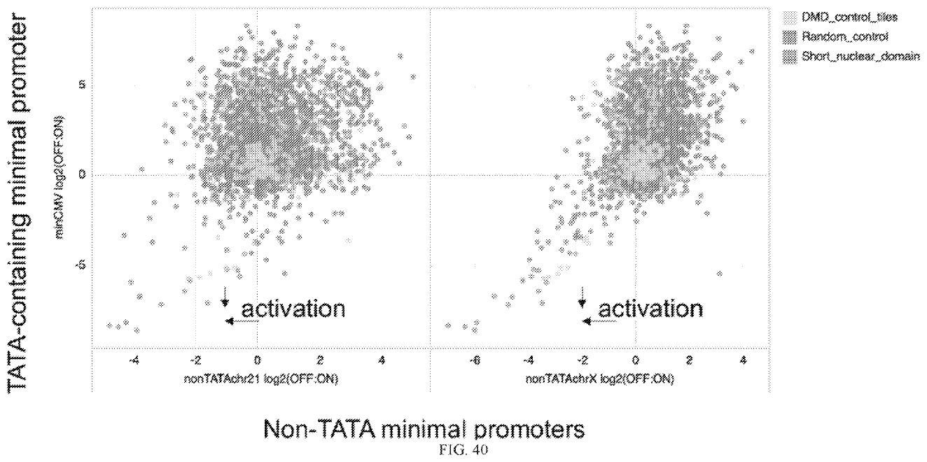

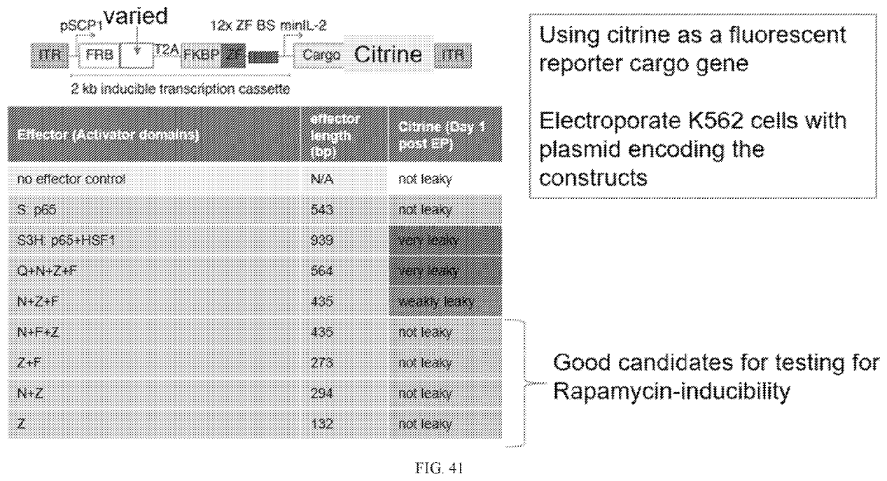

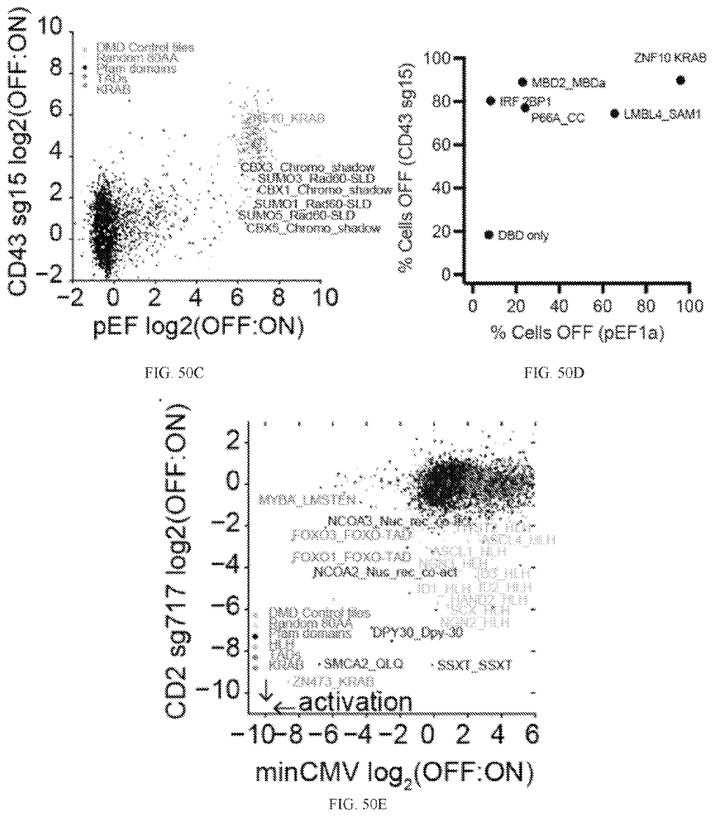

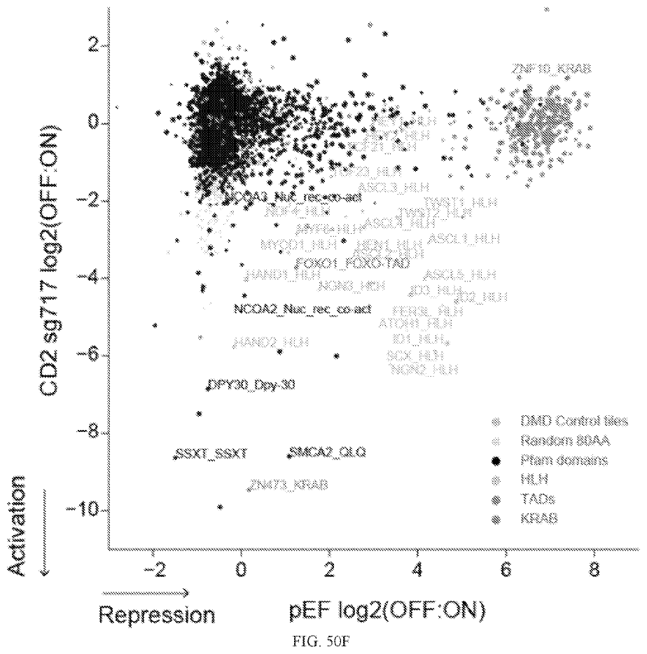

The cell type-specificity of repressors was investigated by comparing the HT-recruit using rTetR to target Pfam domains to the pEF1a reporter in HEK293T and K562 cells. Repression was stronger in K562, both in the screen (FIG. 34, left) and individual measurements (FIGS. 50A and 51A), but the ranking of repressors is similar between cell types. KRAB domains (from ZNF10, ZN777, and ZFP57) and other strong repressors like RYBP and Chromo_shadow that were hits in K562 were also hits in HEK293T cells (FIG. 34, left). A number of medium-strength repressors in K562 become even weaker in HEK293T (FIG. 50A). This category includes CHD3 DUF1087, such that in individual recruitment assays small levels of reporter repression (9.4%) were detected in HEK293T cells (FIG. 51A).

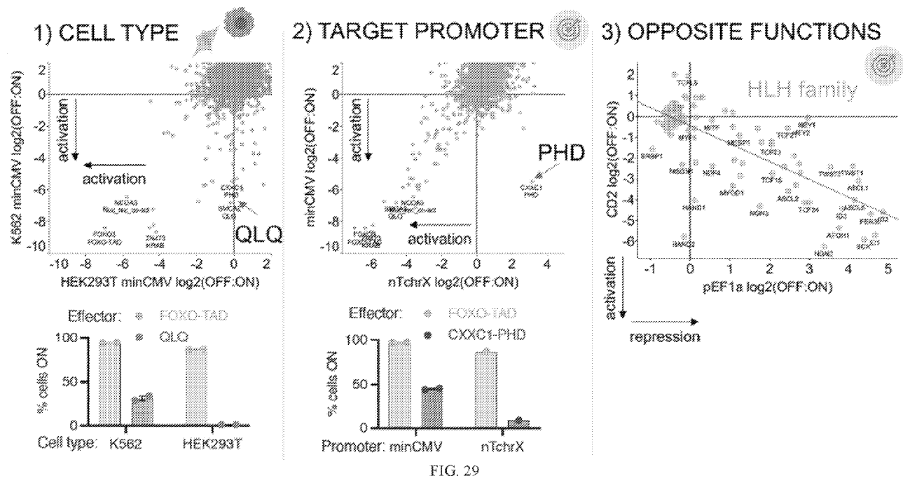

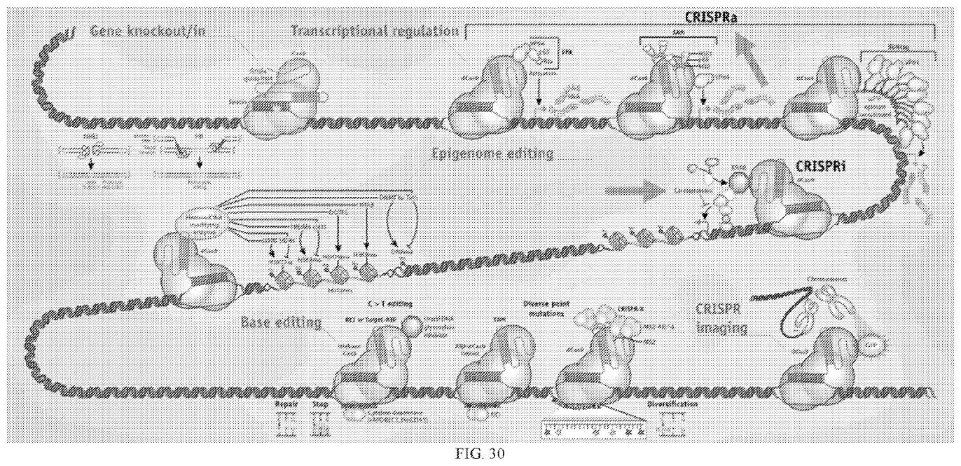

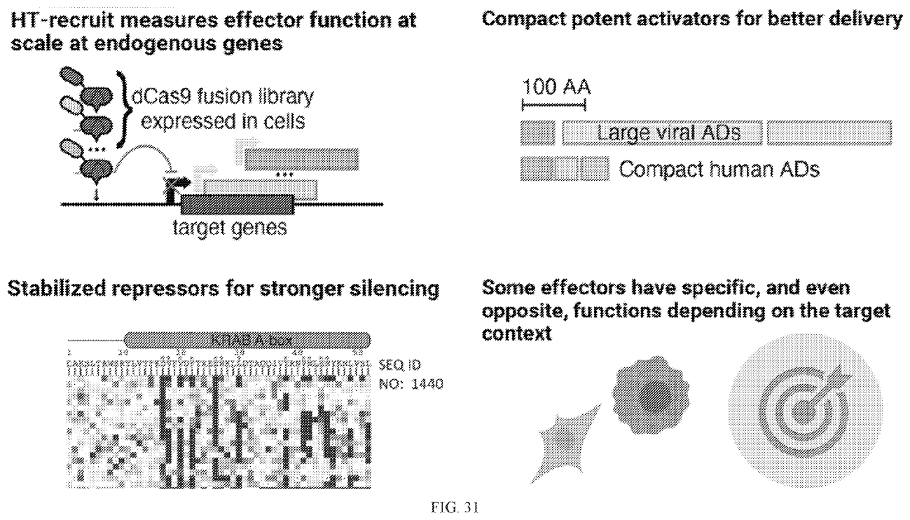

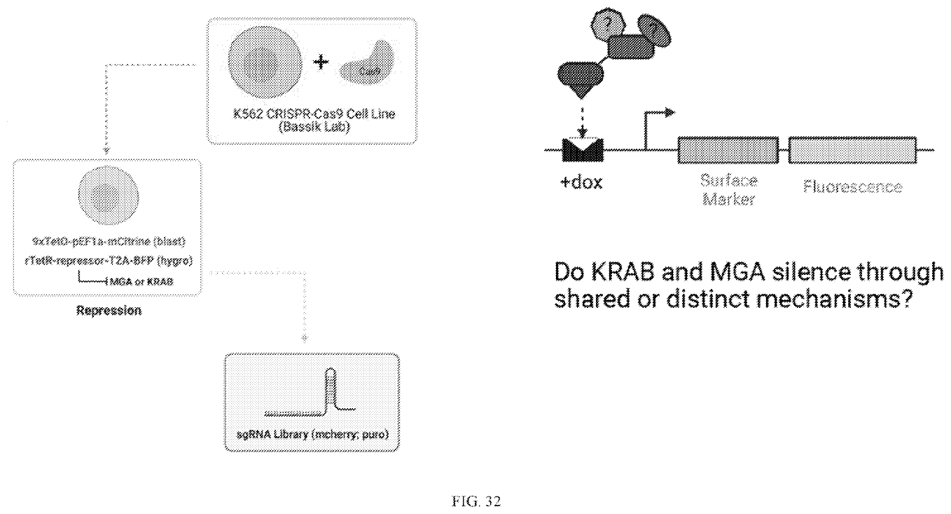

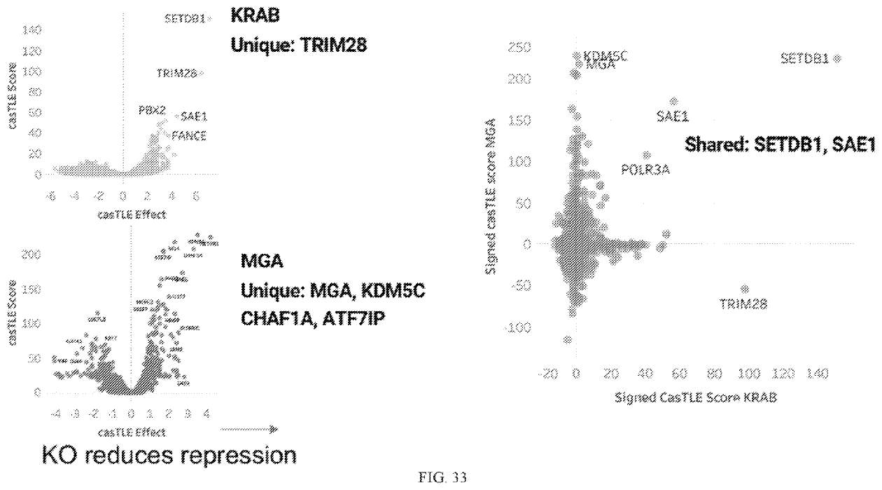

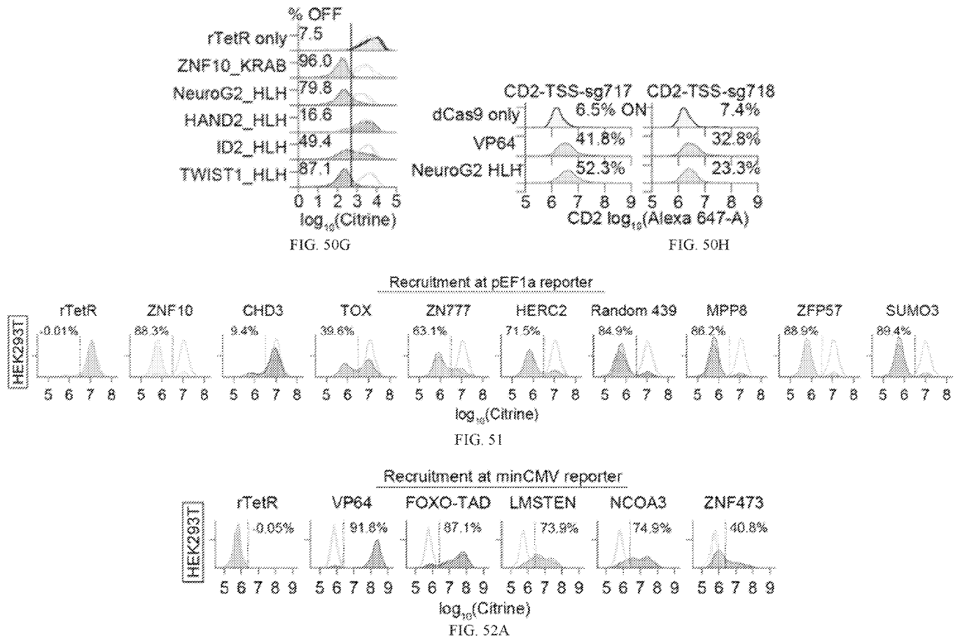

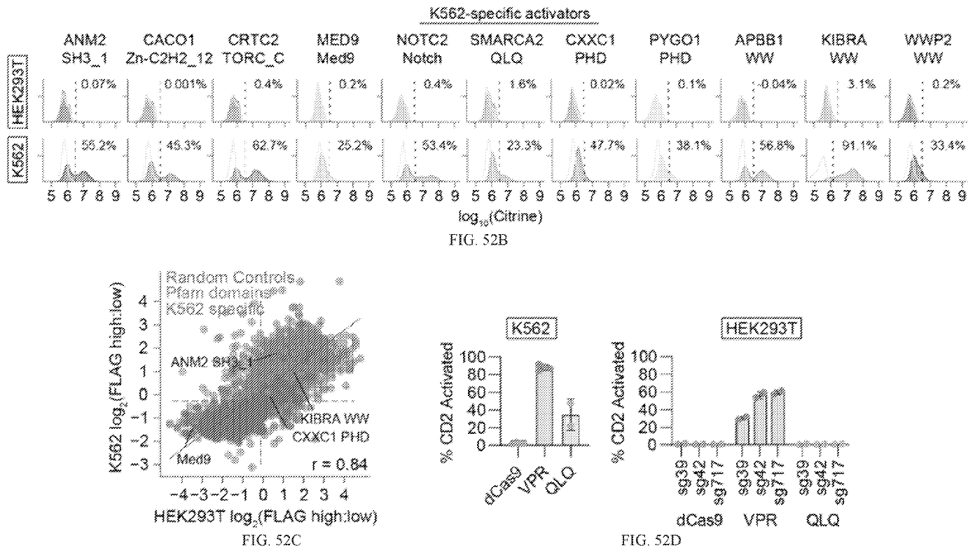

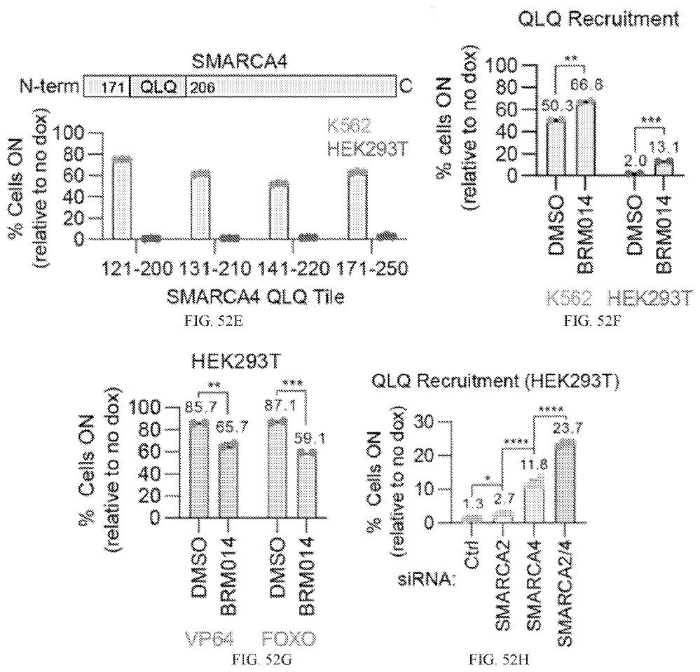

Applying a similar approach, the cell-type specificity of activators was investigated by comparing HT-recruit using rTetR to target the minCMV reporter in HEK293T and K562 cells. A number of activator hits in the K562 screen were also activator hits in the HEK293T screen, including strong activators like FOXO-TAD, LMSTEN, NCOA3, and the ZNF473 KRAB domain (FIGS. 29, top left, and 52A). Of the cell type-specific hits, K562-specific activators, such as ANM2 SH3_1, CACO1 Zn-C2H2_12, CRTC2 TORC_C, MED9 Med9, NOTC2 Notch, SMARCA2 QLQ, PHD domains from CXXC1 and PYGO1, and WW domains from APBB1, KIBRA, and WWP2 (FIG. 29, top left) were observed, which were further validated in individual recruitment assays (FIGS. 50B and 52B). Analysis of the domain expression by FLAG staining revealed that with the exception of the Med9 domain, all the other validated K562-specific activators were highly expressed in both cell types (FIG. 52C), suggesting that these cell-type specific effects may not be due to loss of domain expression in HEK293T cells. Strong HEK293T-specific activators were not identified in this screen.

One of the K562-specific activators identified in the screen is the N-terminus QLQ domain, a Gln-Leu-Gln motif, from SMARCA2. SMARCA2 is a component of the ATP-dependent BAF chromatin remodeling complex involved in regulating transcription by altering the chromatin structure around genes. When fused to dCas9 and targeted to the CD2 endogenous gene, QLQ activated CD2 in K562 but not in HEK293T cells (FIG. 52D), which further suggests QLQ's cell type-specific activity is not dependent on DNA binding domain (DBD) type or genomic site. Moreover, the QLQ domain from SMARCA4 (not included in the Pfam domain library), which shares ˜81% homology with SMARCA2 QLQ, was also a K562-specific activator (FIG. 52E). To explore what might be causing this QLQ's cell-type specific activity, the cells were treated with the SMARCA2/4 ATPase activity inhibitor BRM014 to see its effect on QLQ activation at the minCMV reporter. Unexpectedly, SMARCA2/4 inhibition by BRM014 led to an increase in QLQ activation in both K562 and HEK293T cells at the reporter (FIG. 52F) whereas BRM014 treatment decreased activation by VP64 and FOXO (FIG. 52G). Knockdown of both SMARCA2 and 4 by siRNA further confirmed these results (FIG. 52H).

In addition, WW domains from multiple TFs (KIBRA, APBB1, WWP2) and PHD domains (from PYGO1 and CXXC1) were activators in K562 and not in HEK293T cells (FIGS. 50B and 52B). The FLAG-tag measurements showed this difference in activation was not simply due to differential rTetR-effector expression (FIG. 52C). Both the WW domains (n=6 K562-specific hits) and SH3 domains (n=2 K562-specific hits) were annotated to interact with proline-rich motifs, which are common motifs in transcriptional activators.

### Example 12

### Recruitment at Different Target Genes

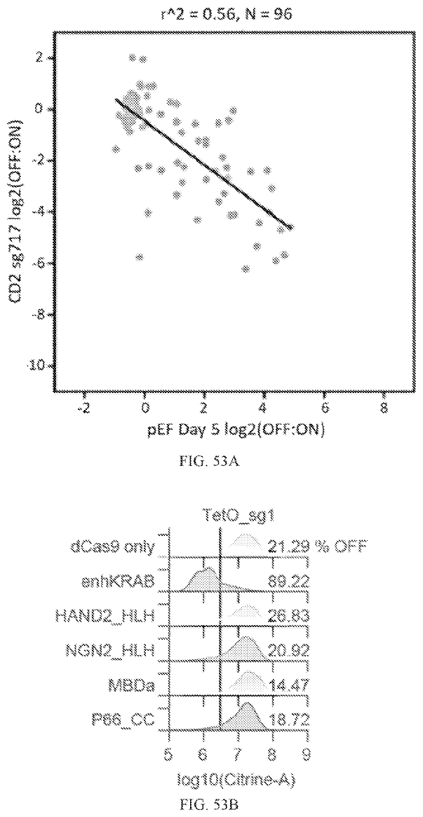

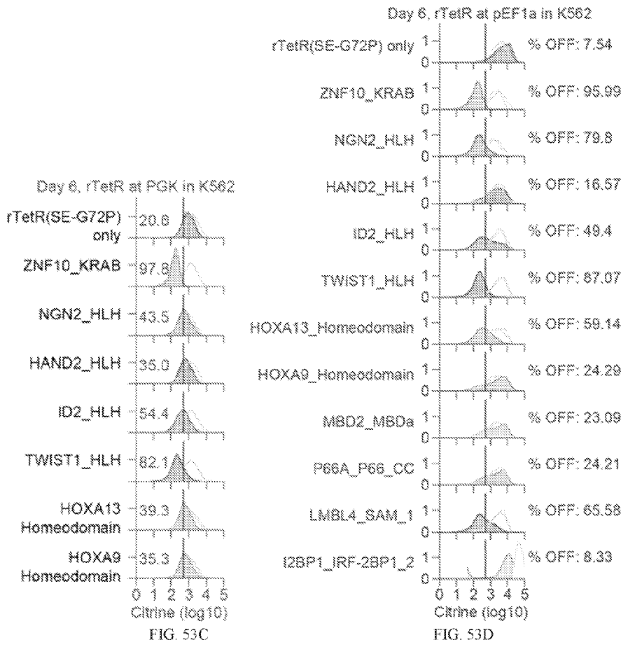

Since more effector domains work in K562 versus HEK293T, K562 cells were predominantly used to investigate how the activity of activators and repressors change at different promoters. Repressor promoter-specificity was investigated using rTetR to target the library members to pEF, PGK, and UbC promoters in K562 cells. Repression at PGK and UbC was highly correlated (FIG. 53), whereas pEF was uniquely silenced by more moderate non-KRAB repressors than PGK or UbC, including the homeodomains (FIG. 53). Similarly, some HLH domains silenced both pEF1a and PGK (e.g., TWIST1 and ID2) while others only silenced pEF1a (e.g., NGN2) (FIG. 46).

In order to expand measurements to endogenous genes, the Pfam library of domains was fused to dCas9 instead of rTetR. The dCas9 fusions were tested by recruiting them at the TetO sites upstream of the pEF reporter. In general, fewer repressor hits were garnered, but the top KRAB domains were still able to repress (FIG. 53C). Using dCas9 to target Pfam domains to the endogenous surface gene CD43, KRAB domains were identified as repressors (FIG. 29, top left). Interestingly, at the CD43 gene a class of repressors were found, the NuRD-interacting Methyl-binding protein domains such as P66_CC and MBDa, that had not previously been uncovered as repressors in the rTetR-mediated HT-recruit screens (FIG. 50B). Meanwhile, the chromoshadow and chromo domains were uniquely hits when recruited with rTetR (FIG. 50B). These context-dependencies held true in individual validations (FIG. 53).

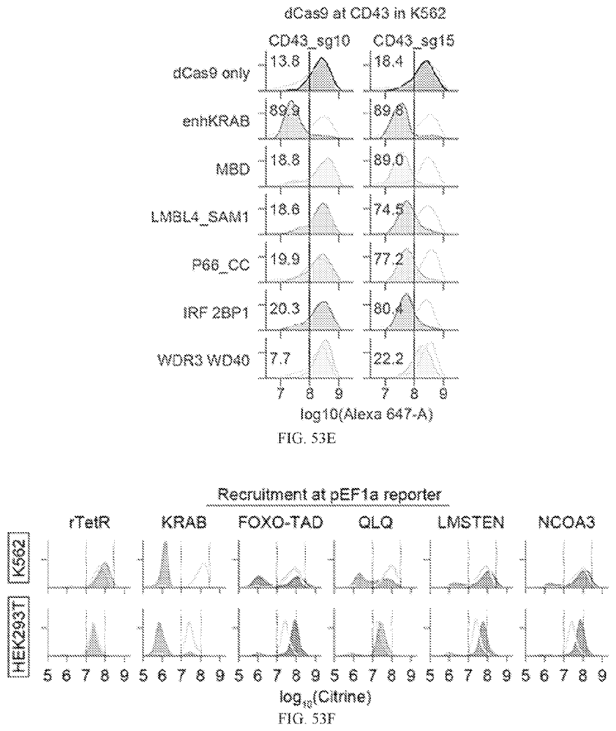

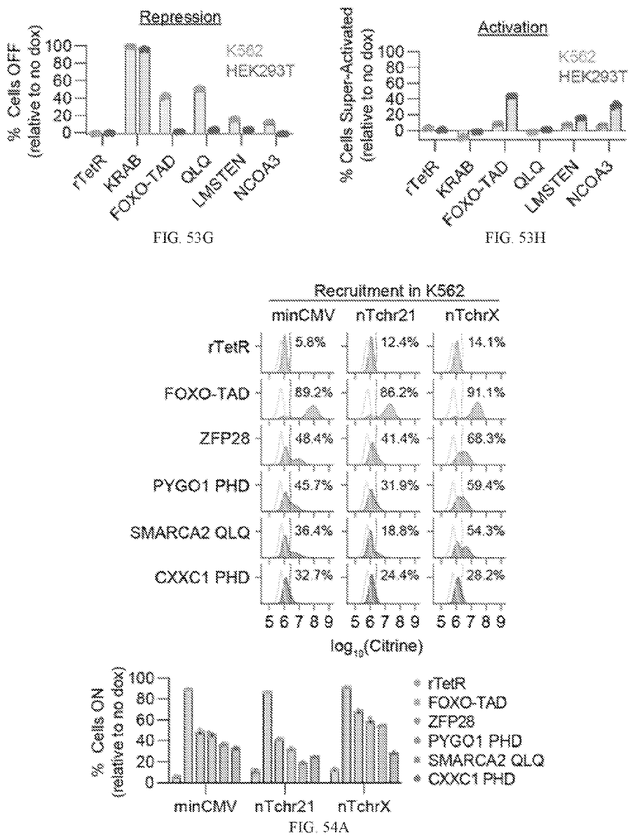

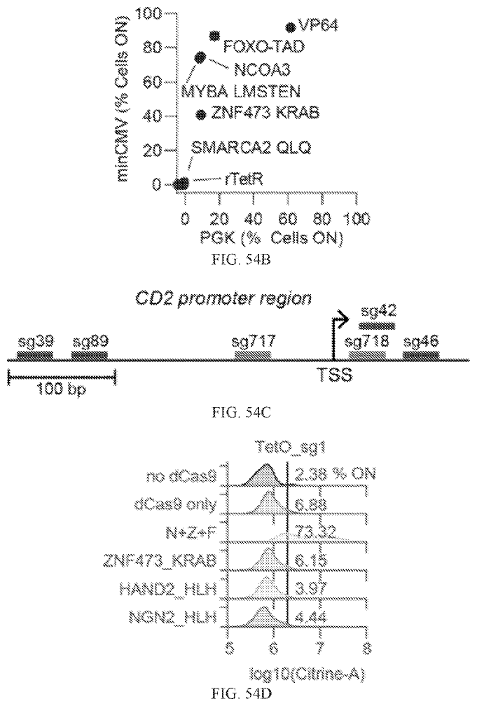

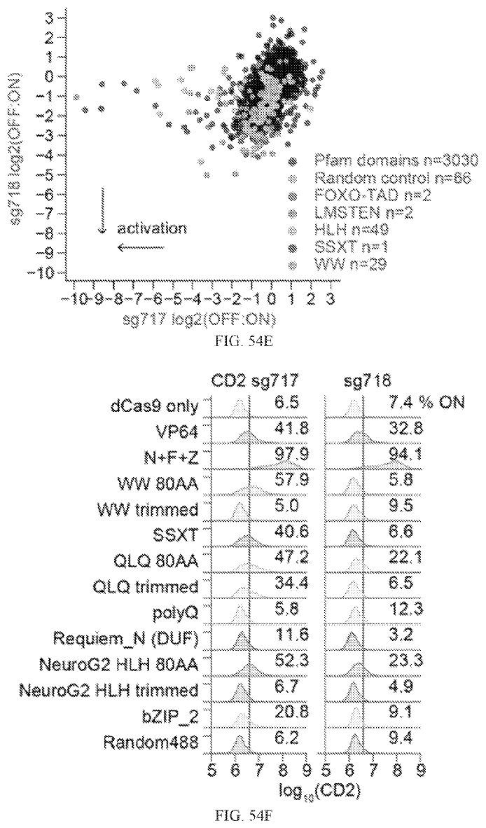

Core promoters were found to respond similarly to activators with rTetR recruitment in K562. Similar activator activity was validated across core promoters (FIG. 54A) and confirmed there are few differences between minCMV, which contains a TATA box, and the two non-TATA core promoters, especially with strong activators such as FOXO-TADs. However, ZFP28, PYGO1 PHD, SMARCA2 QLQ more strongly activated the nTchrX promoter than others (FIG. 54A). The CXXC1 PHD domain was a weak activator across all promoters. In addition, some of these strong activators were able to activate the background silenced PGK promoter in HEK293T cells albeit at a weaker level (FIG. 54B). However, when targeting endogenous genes and changing the DBD, more activator hits were found, including a large family of HLH domains that activate CD2 upon dCas9 recruitment but were not hits when fused to rTetR (FIG. 50E).

It is generally thought that effector domains are classified as activators or repressors; however, like the transcription factors that consists of these domains, these effectors may have a dual activator-repressor function that is context-dependent on gene target or promoter. HT-recruit results were compared from repressible and activatable targets. Many helix-loop-helix (HLH) domains activate the CD2 endogenous gene upon dCas9 recruitment with the sg717 guide in K562 cells (FIG. 50E). The HLH domain is the third largest family of effectors in transcription factor. Moreover, one of the strongest HLH activator domains found was in HAND2 and, previously, recruitment of full length HAND2 with dCas9 was found to be an activator. However, these same HLH domains did not activate the minCMV promoter reporter with dCas9 (FIG. 54A) or rTetR (FIG. 50E). HLH domains only activated CD2 specifically with certain sgRNAs (FIG. 54B) and targeting some of these HLH domains to other endogenous genes (CD20 and CD28) did not lead to activation (FIG. 54C), suggesting that the HLH domains are target- and guide-specific. The HLH effector domains were the largest family of activators that was previously unidentified when using rTetR-mediated HT-recruit and was only discovered by dCas9 recruitment to CD2. Another (non-HLH) activator of CD2 and not minCMV was SSXT, which is a domain from the SWI/SNF chromatin remodeler that interacts with the nucleosome, suggesting that this CD2 activation could be associated with nucleosome interactions. Recently, it was found that a portion of the HLH domain from yeast CBF1 interacts with nucleosomes while the PHO4 HLH does not. The CD2 TSS region was occupied by nucleosomes in K562.

Surprisingly, many of the same HLH effectors can repress the constitutive pEF1a promoter when recruited with rTetR (FIG. 50F). The strength of HLH repression of the pEF1a promoter correlated positively with its strength as an activator at the CD2 gene (R²=0.56, n=96 domains) (FIG. 50F). The HLH family is made up of 6 subfamilies (Group A-F) which functions in related ways; for example, HES subfamily are only repressors, HAND subfamily are only activators, ID subfamily HLH are both, and many other subfamilies did neither (FIG. 50G). In particular, HLH effectors from Group A and D subfamilies can both activate and repress according to a previous phylogenetic classification analysis. Both activation and repression were individually validated from the same HLH effector (FIGS. 50G-50H) and the activation and repression depended on using the full 80 amino acid sequence centered on the HLH domain, whereas trimming to just the Pfam annotated HLH domain was not sufficient. While HLH effectors were only found as activators at CD2, a subset of the HLH effectors that repress pEF1a also repressed the PGK promoter.

There are other cases of effectors that activate in one context and repress in another as identified through the screen (FIG. 54B). For example, FOXO-TAD and QLQ can both activate minCMV and moderately repress pEF1a in K562 cells (FIG. 52E, top). However, this dual activator-repressor function was not observed in HEK293T cells (FIG. 52E, bottom). In contrast, other strong activators such as LMSTEN and NCOA3 did not repress in either K562 and HEK293T cells (FIG. 52E), suggesting this repression was due to the property of the domain and not due to transcriptional interference from any strong activator recruited upstream of pEF1a.

### Example 13

Synergistic Repression with RYBP-MGA

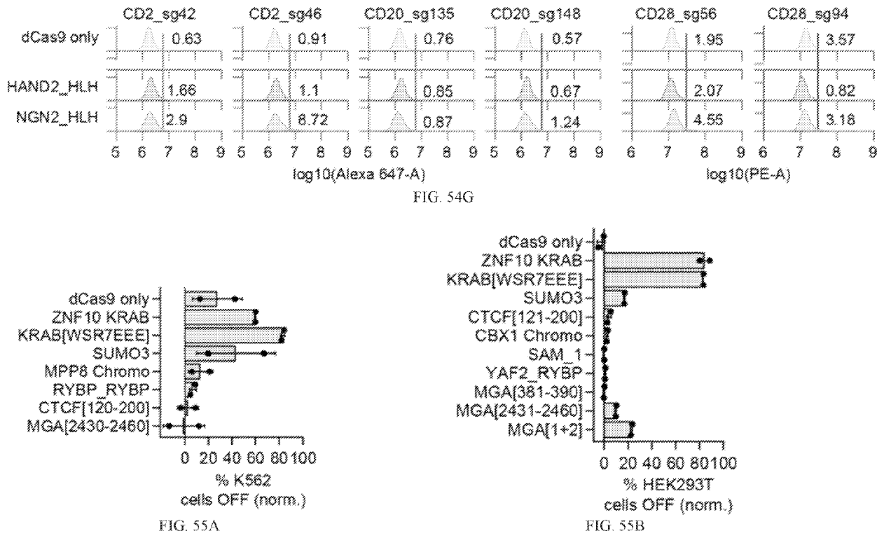

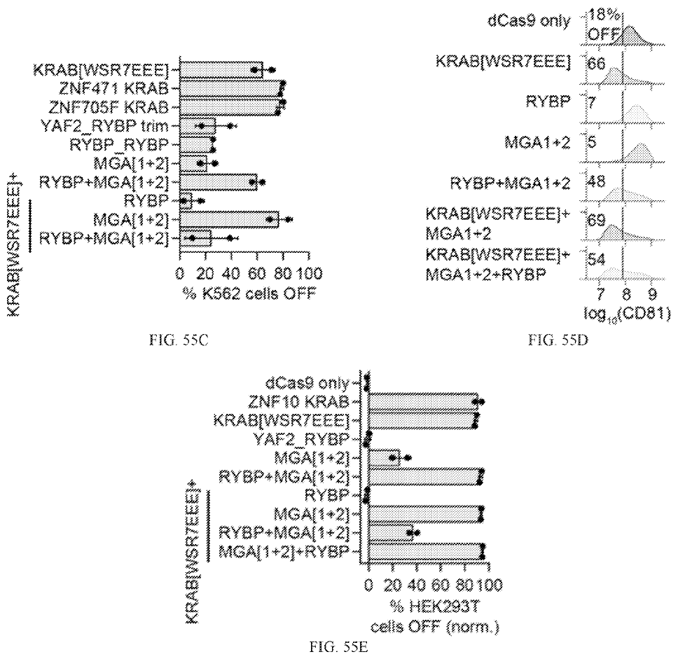

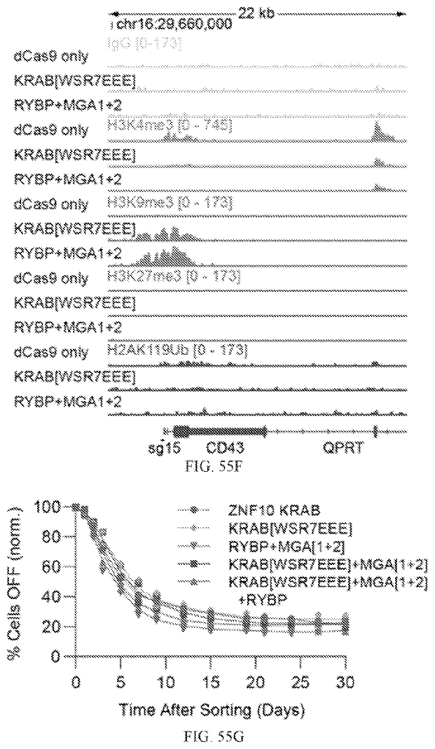

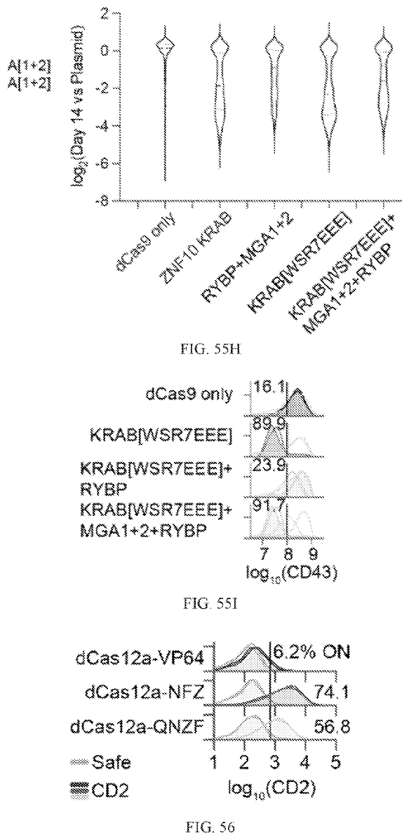

Non-KRAB repressors have been used for gene silencing and epigenomics, but these have generally not demonstrated a similar efficiency as KRAB for CRISPRi applications. In the HT-recruit screens, non-KRAB repressors were less robust across contexts (FIGS. 48 and 50). Accordingly, in individual assays, when fused to dCas9, many repressor domains that are strong when recruited with rTetR in K562, including RYBP and MGA, were much weaker than KRAB when targeted to CD43 in K562 cells (FIG. 55A), or to a reporter in HEK293T cells (FIG. 55B). One mixed case was SUMO3, which silenced CD43 with KRAB-like efficiency using the guide sg15, but it did not silence with sg10, which is in agreement with the results for SUMO3 from the dCas9 HT-recruit screens with these two guides (FIGS. 48 and 55A). Despite these initial difficulties, these repressors could work (e.g., on rTetR) and that they depended on different co-repressors than KRAB (e.g., not KAP1).

Others have reported enhancement of silencing by combining multiple repressors with ZNF10 KRAB and even reported KRAB-like efficiencies with MBD2B repressor fusions, combinations of these non-KRAB repressors were rested. First, when MGA [381-390] and MGA[2431-2460] were combined together, super-additive repression (23% versus 0% and 10% individually) was observed at the HEK293T reporter (FIG. 55B). Then, further combining RYBP with MGA[1+2] (MGA) resulted in super-additive repression both at endogenous CD43 and CD81 in K562 cells and at the HEK293T reporter (93% versus 0% and 26% individually) (FIGS. SSC-55E). CUT&RUN was used to measure how this RYBP-MGA combination modified chromatin at the target CD43 locus, and similar modifications as mediated by the strong KRAB [WSR7EEE] repressor were found (FIG. 55F). Specifically, there was a local deposition of heterochromatic mark H3K9me3 in the 5 kb region around the target, while the activation-associated H3K4me3 mark was locally ablated and partially reduced at a distal site 16 kb away. While some previous literature associates MGA and RYBP with polycomb repression, no changes were observed for polycomb-associated H3K27me3 or H2AK119Ub marks. Moreover, these repressor combinations had the same epigenetic memory as ZNF10 KRAB when targeting the HEK293T reporter (FIG. 55G), consistent with RYBP-MGA mediating similar chromatin modifications as KRAB. However, unlike KRAB, RYBP and MGA do not depend on KAP1, at least for silencing pEF1a.

In a CRISPRi benchmarking screen targeting promoters that were previously shown to be important with a ZNF10 KRAB screen, RYBP-MGA showed much greater efficiency than dCas9 alone but somewhat lower efficiency than ZNF10 KRAB across a number of sgRNAs (FIG. 55H). Together, these results establish RYBP-MGA as an efficient, compact, and context-robust repressor that can be used for CRISPRi applications.

### Example 14

### Compact and Context-Robust Activators for Lentiviral CRISPRa

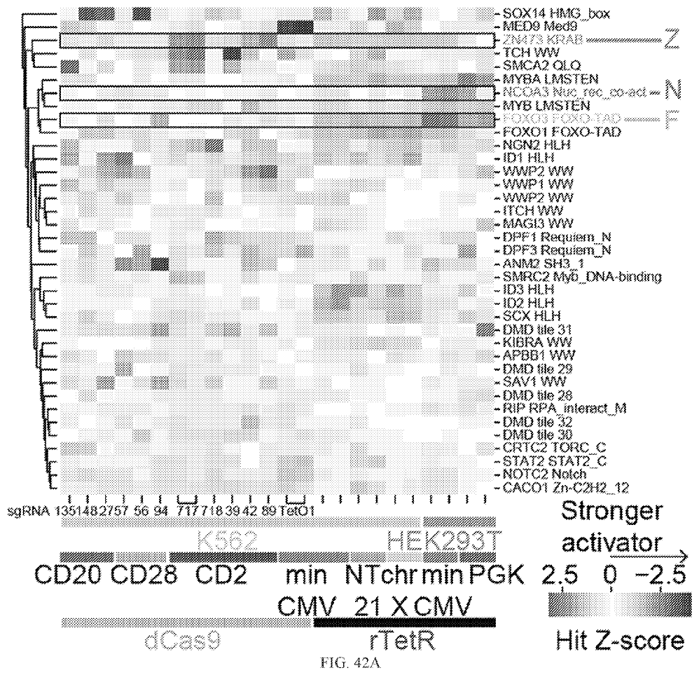

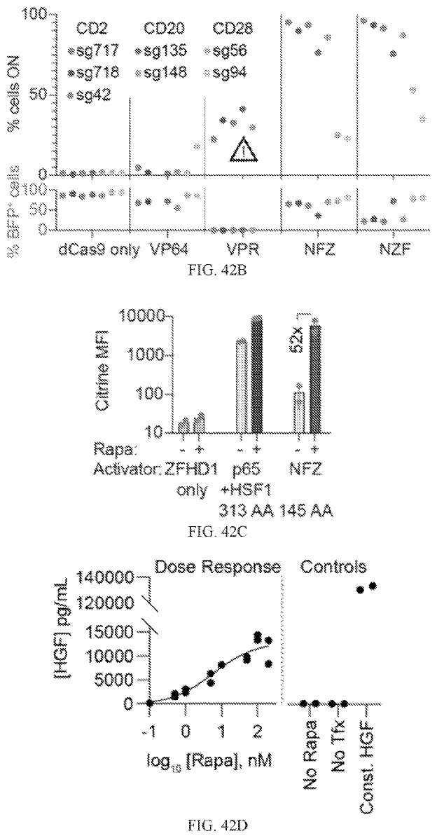

To find the most useful activators for synthetic applications, additional dCas9 HT-recruit screens were performed at the activatable endogenous genes CD20 and CD28, and then activators from the HT-recruit measurements were ranked performed across different contexts (FIG. 42A). The 37 domains that were a hit in at least two activation samples were selected and ranked across contexts, resulting in identification of NCOA3 (N), ZNF473 KRAB (Z), and FOXO3 TAD (F) domains as promising activators (FIG. 42A). ZNF473 KRAB is an ancient, acidic KRAB domain, that is divergent form the modern repressor KRAB domain consensus sequence, and was ranked in the top 50% of these activators across most (22/24) samples, including with both rTetR and dCas9 recruitment. Meanwhile, NCOA3 and FOXO3 TAD were top 50% bits in all 10 rTetR samples. These domains were also well-expressed across DNA binding domains (DBDs) and cell types, and there was no toxicity associated with their expression. The trimmed Pfam-annotated domain for these three activators performed equally to the 80 AA sequence centered on the domain with rTetR. Thus, the more compact Pfam domains (Z=44 AA, N=48 AA, and F=41 AA) were used to build activator tools (FIG. 23A).

These trimmed activators were fused and, as controls, two commonly used activators (VP64 and VPR) were also fused, individually to dCas9 in a lentiviral vector with BFP as a delivery marker. As in the HT-recruit screens, ZNF473 KRAB activated CD2 with sg717 while the other domains were poor activators (FIGS. 42A and 23A). When any of these domains were combined together as bipartite activators (N+Z, N+F, and Z+F), synergistic activation of the CD2 gene was observed (FIG. 23A). Further fusion of all three domains together to create the tripartite activator, NFZ, led to even stronger activation of CD2 (FIG. 23A). At 145 AA in length, NFZ is more compact, does not use viral components, was more efficiently delivered (as marked by BFP), and provided a similarly high level of CD2 activation among BFP+ cells as the 523 AA VPR (FIGS. 23A and 23B).

The most striking feature of NFZ was the improvement in lentiviral delivery with dCas9-NFZ versus dCas9-VPR, as marked by a higher fraction of BFP+ cells (FIG. 23B). Amongst the BFP+ cells, both NFZ and VPR were potent activators. However, there were more BFP+ cells with NFZ than VPR, which could be explained by reduction in initial transduction efficiency and/or subsequent cell toxicity related to VPR's expression. While VPR is the largest effector, the lentiviral insert size (between cPPT and WPRE) is 8.5 kb, which does not exceed packaging limits.

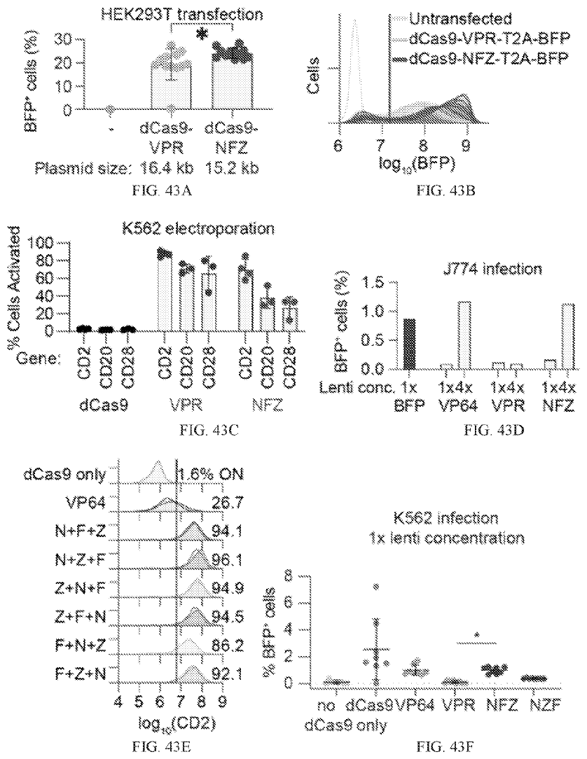

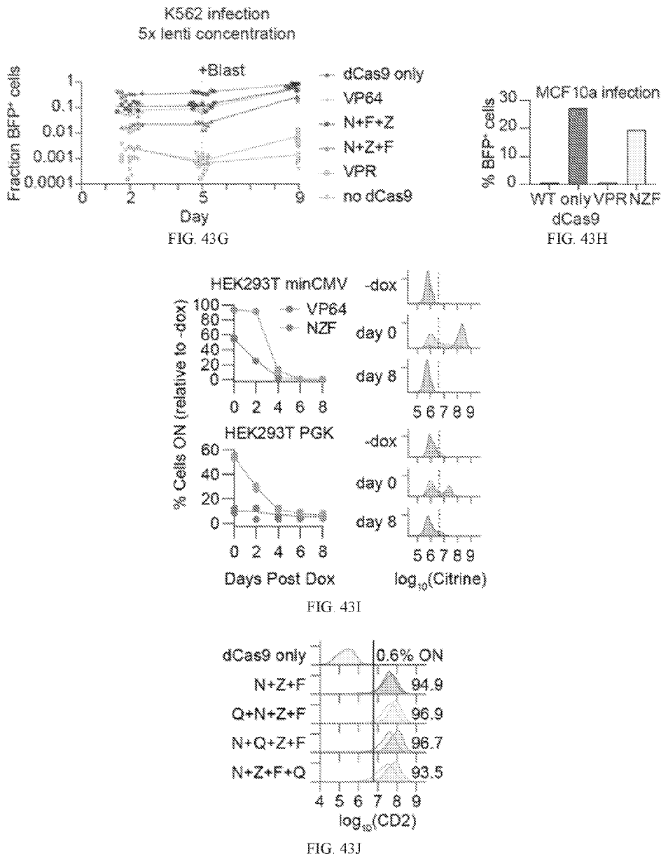

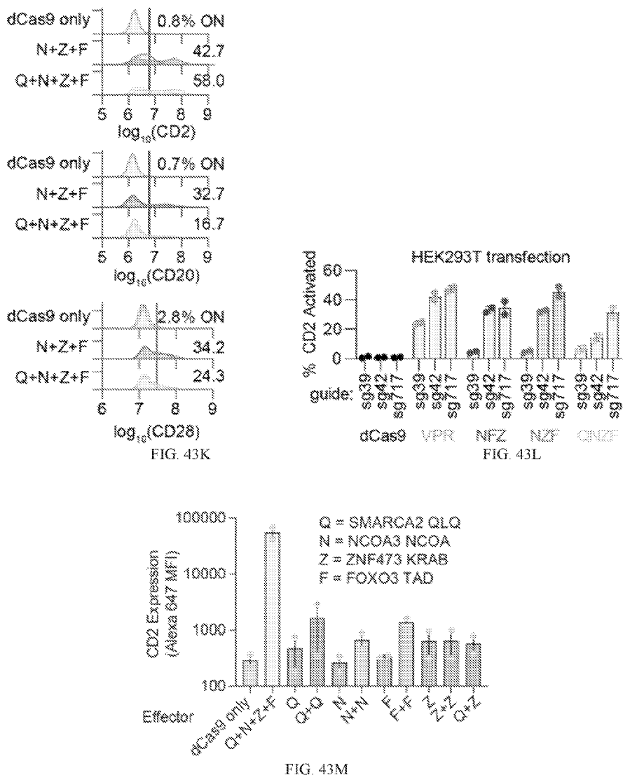

The activity of dCas9-NFZ was confirmed across three endogenous surface marker genes (CD2, CD20, CD28) in K562 cells. NFZ was better delivered than VPR, with unconcentrated or concentrated virus (FIGS. 43F-43G). These cells better survived blasticidin selection, resulting in stable CRISPRa lines with which NFZ activated a higher fraction of cells than VPR (FIG. 42B).

In contrast to what was seen with lentivirus, by plasmid transfection into HEK293T cells, NFZ was similarly delivered as VPR (FIG. 43A). However, the BFP from the dCas9-activator-T2A-BFP-P2A-BlastR transcript was expressed at over 3-fold higher MFI on average with NFZ relative to VPR, when gating on the co-transfected GFP to account fairly for overall delivery efficiency and include only transfected cells (FIG. 43B). By plasmid transfection, VPR transcripts and protein show reduced expression, which could be consistent with squelching or arise from other causes. However, these effects are moderate relative to the large difference observed with lentiviral delivery of NFZ versus VPR. Further, two days after electroporation of the dCas9-activators in K562 cells, VPR provided somewhat better activation of the three surface markers (FIG. 43C), suggesting VPR could be a better tool for transient delivery to cell lines. Meanwhile, lentivirus delivered NFZ to J774 macrophages (FIG. 43D), which are more difficult to infect than K562 cells, and a stable line was successfully selected and sorted with which could activate an endogenous gene. In contrast, lentiviral delivery of VPR resulted in <0.5% delivery 4 days post-infection, and blasticidin selection and sorting could not be successfully used to generate a stable VPR cell line (FIG. 43D). Together, these results show NFZ is easier to deliver than VPR, especially when using lentiviruses to generate stable lines.

By changing the N, F, Z domain orientation, N+Z+F was identified as a slightly stronger activator combination than other configurations, with lentiviral delivery efficiencies between VPR and NFZ (FIGS. 42B and 43E-43G and 43L).

To further test the limits of combining activators, another short activator domain, SMARCA2 QLQ (FIG. 5A), was fused to NZF. The resulting Q+N+Z+F was a stronger activator than NZF at the CD2 gene, but this improvement did not hold at CD20 or CD28, which was consistent with QLQ alone activating CD2 and not CD20 or CD28 in the HT-recruit screens (FIGS. 43J-43K and 42A). Further, consistent with QLQ being a stronger activator in K562 than HEK293T in the screens and validations (FIGS. 50D and 42A), QNZF did not improve activation in HEK293T (FIG. 43L). These activator combinations were also used with dCas12a (FIG. 56).

In addition, the Q, N, and F (but not Z) domains can synergistically activate CD2 when fused as homotypic bipartite combinations (FIG. 43M). However, heterotypic combinations lack repetitive sequences and should be simpler to synthesize and package in viral vectors.

Inducible expression with the compact tripartite activator This combination was ported to the rTetR DBD and in the presence of dox, rTetR-NZF activated minCMV or partially reactivated the background-silenced PGK reporter in HEK293T cells (FIG. 43H). While rTetR and dox are useful for research applications, there is a longstanding interest in making AAV transgene expression inducible by using a more fully humanized synthetic transcription factor that conditionally activates a minimal promoter in the presence of a safe inducer small molecule. Such a concept has been implemented using the zinc finger-homeodomain DBD (ZFHD1) and the rapamycin-inducible FKBP-dimerization system. When functionalized with the well-known P65 and HSF1 activators, this system has been demonstrated to reliably control transgene expression for as long as 6 years post-delivery in a non-human primate model. However, it has limited applicability for many diseases because the large size of the activator constructs leaves only 1.1 kb of coding capacity for the therapeutic transgene (while remaining within the 4.7 kb packaging capacity of AAV).

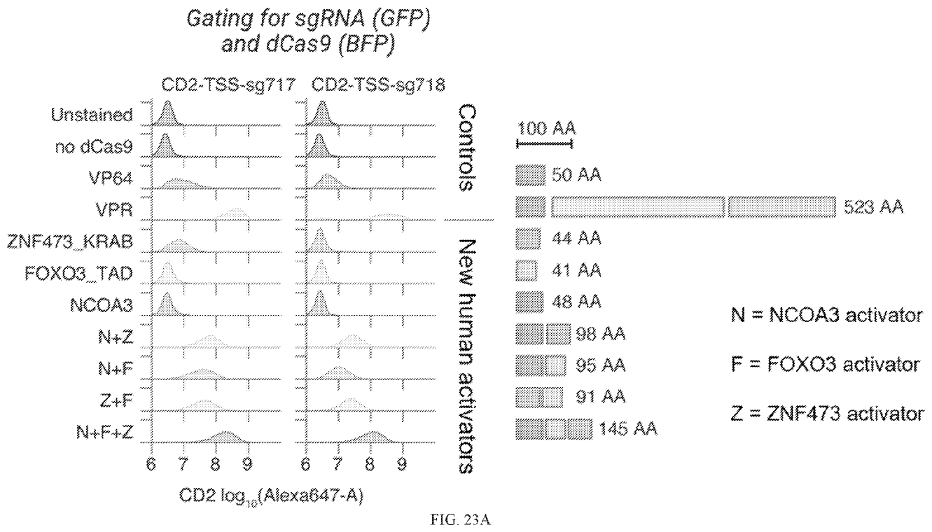

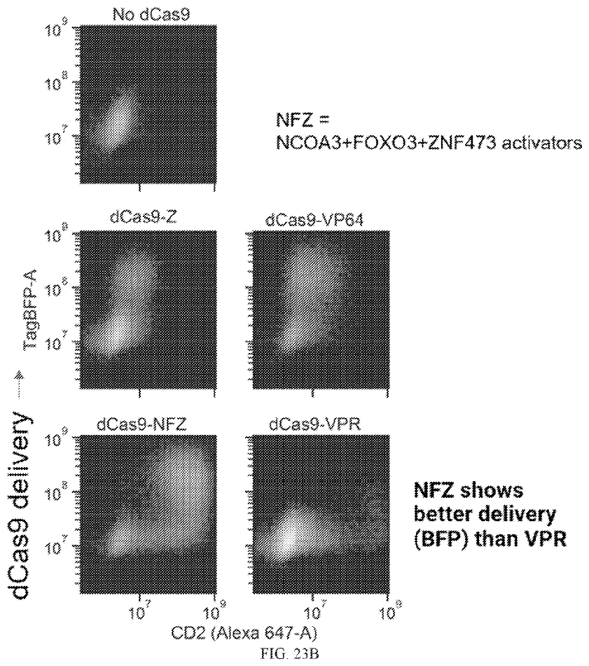

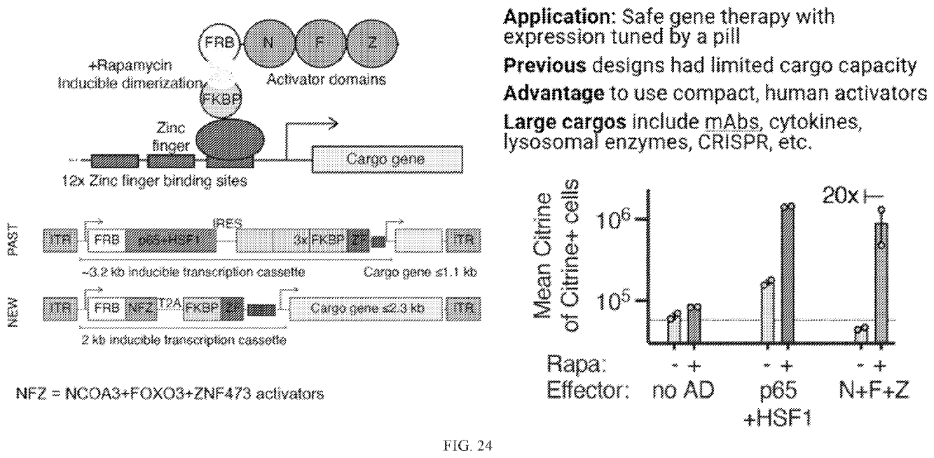

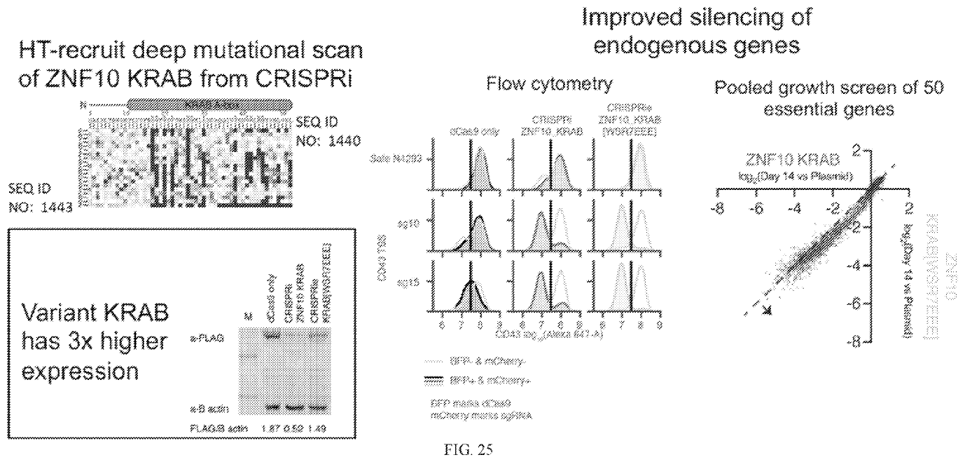

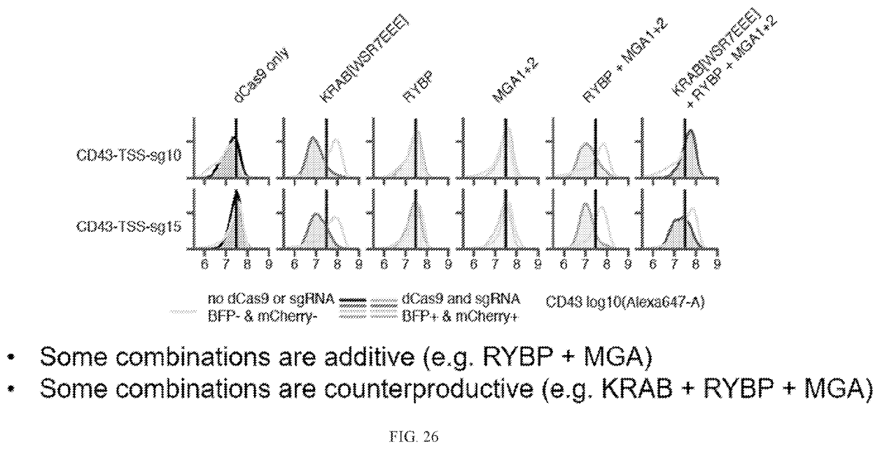

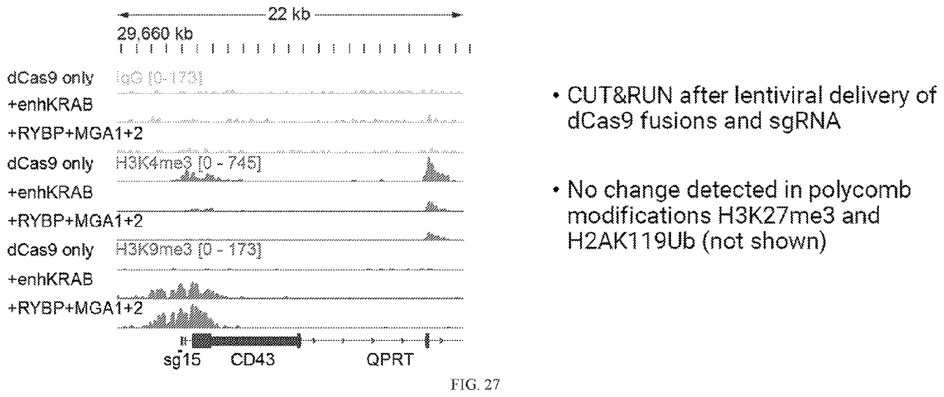

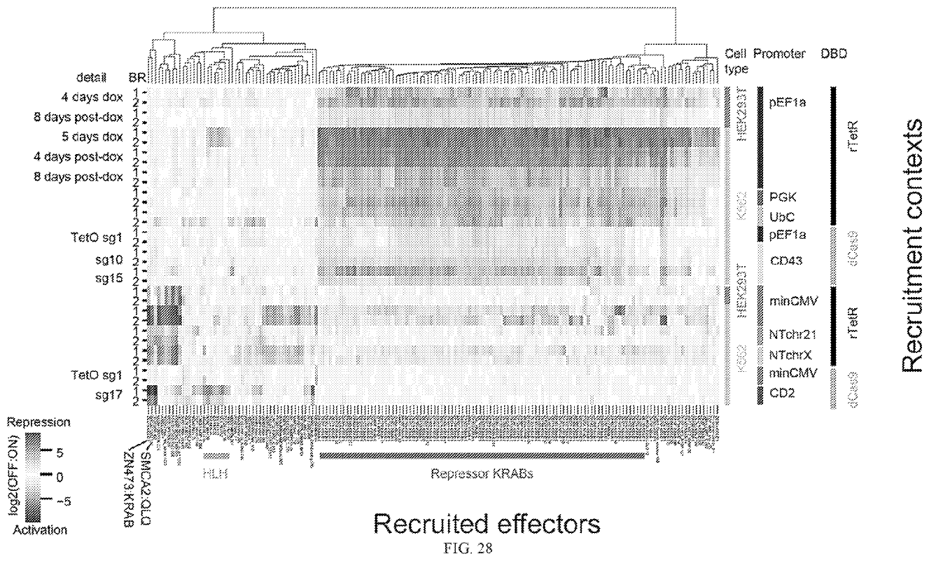

A new compact inducible AAV system by using the following components: (1) the compact NFZ activator, (2) minimal promoters and terminators, and (3) fewer repeats of FKBP (with the assumption fewer FKBP repeats are needed if the activators are stronger). In sum, these modifications expanded the transgene cargo capacity from 1.1 to 2.3 kb (FIG. 24), which is enough to encode about 22% of genes in the human genome (FIG. 24).

As an initial test, the newly designed inducible AAV construct that contains a Citrine transgene was transiently transfected. Rapamycin-dependent Citrine inducibility and low levels of leakyness were observed with NFZ, whereas in the previously used P65+HSF1 there was high level of leakiness and with reduced transgene cargo capacity (FIG. 42C). Expanding upon this work, the Citrine transgene was swapped for the larger ˜2.2 kb human hepatocyte growth factor (HGF) gene (FIG. 44A). HGF is a pleiotropic secreted factor whose therapeutic potential in a wide range of human disease has been investigated in preclinical disease models, such as fibrosis, and type 1 diabetes, and has been approved as a plasmid gene therapy for treatment of critical limb ischemia in Japan. However, it's in vivo utility is limited by (1) its short half-life of 3-5 mins which limits its therapeutic effect, (2) its size has precluded its inclusion in previous inducible AAV constructs, and (3) its well-controlled delivery remains a challenge. Moreover, constitutive expression of HGF may cause tumorigenesis as overexpression of HGF has been reported for human gastric or pancreatic cancer. Being able to induce HGF expression with a small molecule, such as rapamycin, could overcome these limitations. The HGF inducible construct was transfected in HEK293T cells and expression of HGF was induced for 2 days with rapamycin. By ELISA, a significant and rapamycin dose-dependent increase in secreted HGF protein, with minimal leakiness in untreated cells, was observed (FIG. 5H). Collectively, these results the NFZ activator can reliably be ported to a new context to enable synthetic biology applications.

### Example 15

CRISPR Interference with the ZNF705F KRAB Repressor Domain

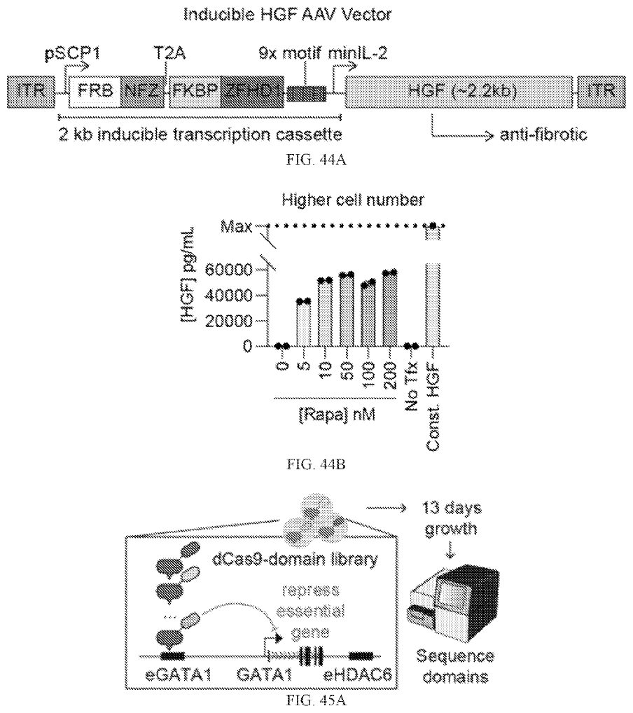

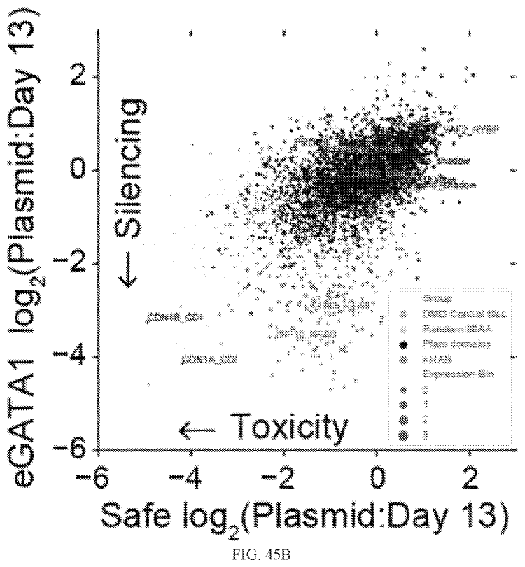

Performing an additional screen at the GATA1 locus allowed comparison of repressors at an enhancer region (FIG. 45A). Since GATA1 is an essential gene, the growth phenotype associated with targeting dCas9-effectors to its TSS or enhancers (eGATA1) was used as a proxy for repression strength. This approach was previously validated by demonstrating a positive correlation between growth phenotype and CRISPRi-mediated GATA1 silencing via Western blot and immunostaining.

Before using growth as a selection strategy for a CRISPR HT-recruit screen, the growth effects of expressing the dCas9-effector fusions, were inspected in a negative control screen with a safe-target sgRNA called N4294. This safe-targeting sgRNA does not cause growth effects when co-expressed with dCas9 or dCas9-KRAB (from ZNF10). In this control screen, some domains were associated with cell toxicity or growth defects, with the strongest being the cyclin-dependent kinase inhibitor (CDI) domains from CDKN1A/B (FIG. 45B), which are known to inhibit cell cycle progression by binding to CDK2. Toxic domains were removed from further consideration in building CRISPRi tools. The growth effect of each dCas9-effector was considered when paired with the eGATA1 enhancer-targeting sgRNA (FIG. 45B).

To identify strong KRAB repressors that function well across many different contexts, the KRAB paralogs were ranked across all the HT-recruit screens and in addition to ZNF705F KRAB, the KRAB domain from ZNF471 was identified as another top repressor (FIG. 46A). This approach also identified ZIM3 KRAB as a stronger repressor than ZNF10 KRAB. Further, ZNF473 KRAB, which was previously identified as a rare KRAB activator domain, was the last-ranked KRAB in terms of repression (FIG. 46A).

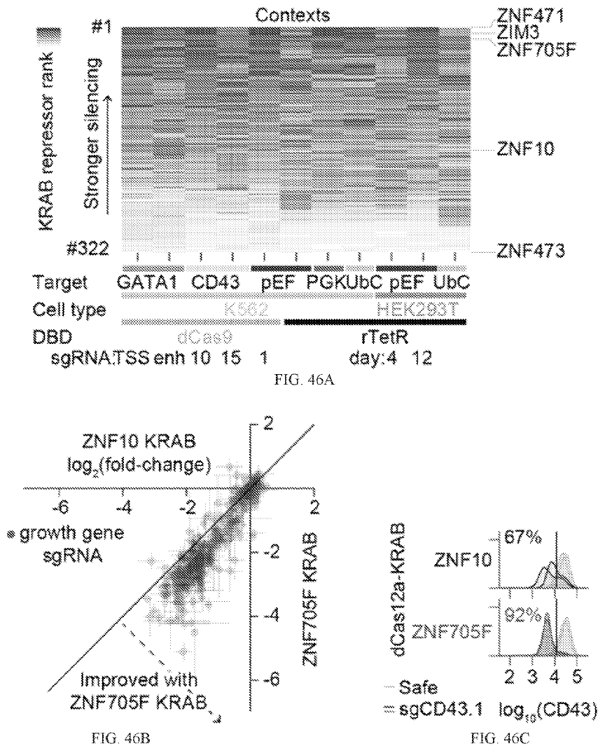

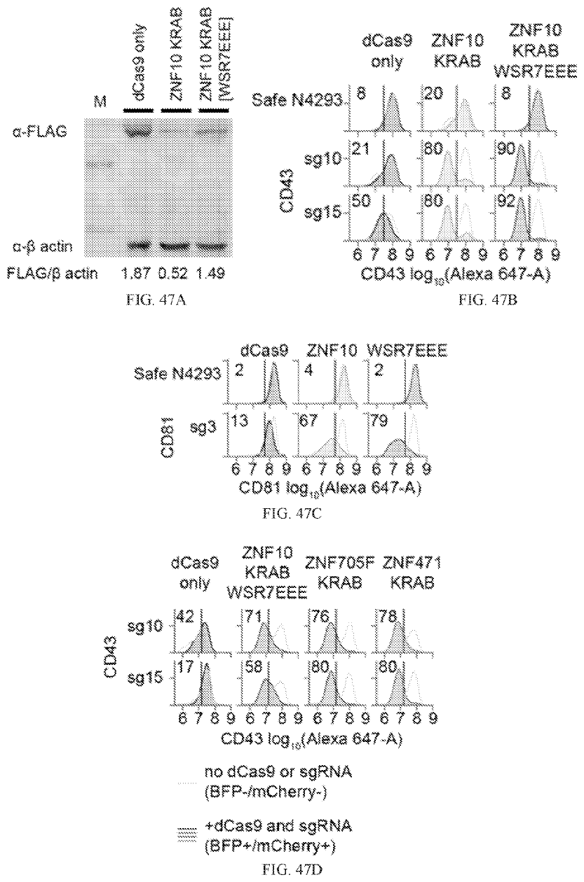

Previously, using a deep mutational scan of the ZNF10 KRAB, a mutant, WSR7EEE, in the N-terminal KRAB domain region was identified as providing increased expression in cells and silencing strength when fused to rTetR. The same was true when this mutant was fused to dCas9 (FIGS. 47A-47C). This enhanced ZNF10 KRAB,WSR7EEE, was used as a benchmark when determining how well these new KRAB repressor paralogs work.

When targeted to the CD43 TSS with dCas9, both of the newly identified KRAB paralogs outperformed the ZNF10 KRAB[WSR7EEE] (FIG. 47D). To determine if this advantage was consistent across a wider range of targets, benchmarking CRISPRi screens were performed with a library of sgRNAs targeting promoters of essential genes (FIG. 47E) and it was found that ZNF705F KRAB consistently provided ˜1.5× greater effect across a range of baseline effects with ZNF10 KRAB at promoters, and was also significantly stronger than the mutant ZNF10[WSR7EEE] (FIGS. 46B and 47F-47G). ZNF705F KRAB was also consistently stronger at perturbing enhancers. To test this repressor in a different cell type, the dCas9-KRAB fusions were transiently transfected with an sgRNA to target the pEF1a reporter in HEK293T cells, and complete silencing was observed after 5 days (FIGS. 47H-47I).

To assess the KRAB repressors in a different DBD context where there is more room to improve efficiency beyond that of ZNF10 KRAB, dCas12a, a compact CRISPR DBD which is generally weaker than dCas9 and which was not included in the HT-recruit screens was used. With dCas12a, it was similarly observed that ZNF705F KRAB recruitment consistently resulted in ˜1.5× more complete silencing of a population of cells than ZNF10 KRAB (FIGS. 46C and 47J). The mutant ZNF10[WSR7EEE] improved silencing by increasing CRISPRi expression level. These results confirm ZNF705F KRAB was a consistently superior repressor for CRISPRi applications and demonstrated how the effector-by-context map facilitates the discovery of robust effectors that can be successfully ported across genomic, cell-type, and DBD contexts. Accordingly, other even higher-ranked KRABs in the map, including ZNF471 and ZIM3 KRABs, should provide improved performance.

### Materials and Methods

Cell lines and cell culture All experiments were carried out in K562 cells (ATCC CCL-243). Cells were cultured in a controlled humidified incubator at 37° C. and 5% CO2, in RPMI 1640 (Gibco) media supplemented with 10% FBS (Hyclone), penicillin (10,000 I.U./mL), streptomycin (10,000 μg/mL), and L-glutamine (2 mM). HEK293FT and HEK293T-LentiX cells were grown in DMEM (Gibco) media supplemented with 10% FBS (Hyclone), penicillin (10,000 I.U./mL), and streptomycin (10,000 μg/mL) and used to produce lentivirus. Reporter cell lines were generated by TALEN-mediated homology-directed repair to integrate a donor construct into the AAVS1 locus as follows: 1.2×10⁶ K562 cells were electroporated in Amaxa solution (Lonza Nucleofector 2b, setting TO-16) with 1000 ng of reporter donor plasmid, and 500 ng of each TALEN-L (Addgene #35431) and TALEN-R (Addgene #35432) plasmid (targeting upstream and downstream the intended DNA cleavage site, respectively). After 7 days, the cells were treated with 1000 ng/mL puromycin antibiotic for 5 days to select for a population where the donor was stably integrated in the intended locus, which provides a promoter to express the PuroR resistance gene. Fluorescent reporter expression was measured by microscopy and by flow cytometry (BD Accuri).

Nuclear protein Pfam domain library design The UniProt database (UniProt Consortium, 2015) was queried for human genes that can localize to the nucleus. Subcellular location information on UniProt was determined from publications or ‘by similarity’ in cases where there was only a publication on a similar gene (e.g., ortholog) and was manually reviewed. Pfam-annotated domains were then retrieved using the ProDy searchPfam function (Bakan et al., 2011). domains that were 80 amino acids or shorter were filtered for and the C2H2 Zinc finger DNA-binding domains, which are highly abundant, repetitive, were excluded and not expected to function as transcriptional effectors. The sequence of the annotated domain was retrieved and it was extended equally on either side to reach 80 amino acids total. Duplicate sequences were removed, then codon optimization was performed for human codon usage, removing BsmBI sites and constraining GC content to between 20% and 75% in every 50 nucleotide window (performed with DNA chisel (Zulkower and Rosser, 2020)). 499 random controls of 80 amino acids lacking stop codons were computationally generated as controls. 362 elements tiling the DMD protein in 80 amino acid tiles with a 10 amino acid sliding window were also included as controls because DMD was not thought to be a transcriptional regulator. In total, the library consists of 5,955 elements.

Silencer tiling library design 216 proteins involved in transcriptional silencing were curated from a database of transcriptional regulators (Lambert et al., 2018). 32 proteins likely to be involved in transcriptional silencing were manually added and then an unbiased protein tiling library was generated. To do this, the canonical transcript for each gene was retrieved from the Ensembl BioMart (Kinsella et al., 2011) using the Python API. If no canonical transcript was found, the longest transcript with a CDS was retrieved. The coding sequences were divided into 80 amino acid tiles with a 10 amino acid sliding window between tiles. For each gene, a final tile was included, spanning from 80 amino acids upstream of the last residue to that last residue, such that the C-terminal region would be included in the library. Duplicate protein sequences were removed, and codon optimization was performed for human codon usage, removing BsmBI sites and constraining GC content to between 20% and 75% in every 50 nucleotide window (performed with DNA chisel (Zulkower and Rosser, 2020)). 361 DMD tiling negative controls were included, as in the previous library design, resulting in 15,737 library elements in total.

KRAB deep mutational scan library design A deep mutational scan of ZNF10 KRAB domain sequence, as used in CRISPRi (Gilbert et al., 2014), was designed with all possible single substitutions and all consecutive double and triple substitutions of the same amino acid (e.g., substitution with AAA). These amino acid sequences were reverse translated into DNA sequences using a probabilistic codon optimization algorithm, such that each DNA sequence contains some variation beyond the substituted residues, which improves the ability to unambiguously align sequencing reads to unique library members. In addition, all Pfam-annotated KRAB domains from human KRAB genes found on InterPro were included, similarly as in the previous nuclear Pfam domain library. Tiling sequences, as designed in the previous tiling library, were also included for five KRAB Zinc Finger genes. 300 random control sequences and 200 tiles from the DMD gene were included as negative controls. During codon optimization, BsmBI sites were removed and GC content was constrained to be between 30% and 70% in every 80 nucleotide window (performed with DNA chisel (Zulkower and Rosser, 2020)). The total library size was 5,731 elements.

Domain library cloning Oligonucleotides with lengths up to 300 nucleotides were synthesized as pooled libraries (Twist Biosciences) and then PCR amplified. 6×50 μl reactions were set up in a clean PCR hood to avoid amplifying contaminating DNA. For each reaction, 5 ng of template, 0.1 μl of each 100 μM primer, 1 μl of Herculase II polymerase (Agilent), 1 μl of DMSO, 1 μl of 10 nM dNTPs, and 10 μl of 5× Herculase buffer was used. The thermocycling protocol was 3 minutes at 98° C., then cycles of 98° C. for 20 seconds, 61° C. for 20 seconds, 72° C. for 30 seconds, and then a final step of 72° C. for 3 minutes. The default cycle number was 29×, and this was optimized for each library to find the lowest cycle that resulted in a clean visible product for gel extraction (in practice, 25 cycles was the minimum). After PCR, the resulting dsDNA libraries were gel extracted by loading ≥4 lanes of a 2% TBE gel, excising the band at the expected length (around 300 bp), and using a QIAgen gel extraction kit. The libraries were cloned into a lentiviral recruitment vector pJT050 with 4×10 μl GoldenGate reactions (75 ng of pre-digested and gel-extracted backbone plasmid, 5 ng of library, 0.13 μl of T4 DNA ligase (NEB, 20000 U/μl), 0.75 μl of Esp3I-HF (NEB), and 1 μl of 10× T4 DNA ligase buffer) with 30 cycles of digestion at 37° C. and ligation at 16° C. for 5 minutes each, followed by a final 5 minute digestion at 37° C. and then 20 minutes of heat inactivation at 70° C. The reactions were then pooled and purified with MinElute columns (QIAgen), eluting in 6 μl of ddH₂O. 2 μl per tube was transformed into two tubes of 50 μl of electrocompetent cells (Lucigen DUO) following the manufacturer's instructions. After recovery, the cells were plated on 3-7 large 10″×10″ LB plates with carbenicillin. After overnight growth at 37° C., the bacterial colonies were scraped into a collection bottle and plasmid pools were extracted with a HiSpeed Plasmid Maxiprep kit (QIAgen). 2-3 small plates were prepared in parallel with diluted transformed cells in order to count colonies and confirm the transformation efficiency was sufficient to maintain at least 30× library coverage. To determine the quality of the libraries, the domains were amplified from the plasmid pool and from the original oligo pool by PCR with primers with extensions that include Illumina adapters and sequenced. The PCR and sequencing protocol were the same as described below for sequencing from genomic DNA, except these PCRs use 10 ng of input DNA and 17 cycles. These sequencing datasets were analyzed as described below to determine the uniformity of coverage and synthesis quality of the libraries. In addition, 20-30 colonies from the transformations were Sanger sequenced (Quintara) to estimate the cloning efficiency and the proportion of empty backbone plasmids in the pools.

High-throughput recruitment to measure repressor activity Large scale lentivirus production and spinfection of K562 cells were performed. To generate sufficient lentivirus to infect the libraries into K562 cells, HEK293T cells were plated on four 15-cm tissue culture plates. On each plate, 9×105 HEK293T cells were plated in 30 mL of DMEM, grown overnight, and then transfected with 8 μg of an equimolar mixture of the three third-generation packaging plasmids and 8 μg of rTetR-domain library vectors using 50 μl of polyethylenimine (PEI, Polysciences #23966). After 48 hours and 72 hours of incubation, lentivirus was harvested. The pooled lentivirus was filtered through a 0.45-μm PVDF filter (Millipore) to remove any cellular debris. For the nuclear Pfam domain repressor screen, 4.5×10⁷ K562 reporter cells were infected with the lentiviral library by spinfection for 2 hours, with two separate biological replicates of the infection. Infected cells grew for 3 days and then the cells were selected with blasticidin (10 μg/mL, Sigma). Infection and selection efficiency were monitored each day using flow cytometry to measure mCherry (BD Accuri C6). Cells were maintained in spinner flasks in log growth conditions each day by diluting cell concentrations back to a 5×10⁵ cells/mL, with at least 1.5×10⁸ cells total remaining per replicate such that the lowest maintenance coverage was >25,000× cells per library element (a very high coverage level that compensates for losses from incomplete blasticidin selection, library preparation, and library synthesis errors). On day 6 post-infection, recruitment was induced by treating the cells with 1000 ng/ml doxycycline (Fisher Scientific) for 5 days, then cells were spun down out of doxycycline and blasticidin and maintained in untreated RPMI media for 8 more days, up to Day 13 counting from the addition of doxycycline. 2.5×10⁸ cells were taken for measurements at each timepoint (days 5, 9, and 13). The protocol was similar for the KRAB DMS, but doxycycline was added on day & post-infection, >12,500× coverage, and 2×10⁸-2.2×10⁸ cells were taken for each timepoint. The protocol was similar for the tiling screen, but 9.6×10⁷ cells were infected, doxycycline was added on day 8 post-infection, at least 2×10⁸ cells were maintained at each passage for >12,500× coverage, and 2×10⁸-2.7×10⁸ cells were taken for each timepoint.

High-throughput recruitment to measure transcriptional activation activity For the nuclear Pfam domain activator screen, lentivirus for the nuclear Pfam library in the rTetR(SE-G72P)-3×FLAG vector was generated as for the repressor screen, and 3.8×10⁷ K562-pDY32 minCMV reporter cells were infected with the lentiviral library by spinfection for 2 hours, with two separate biological replicates of the infection. Infected cells grew for 2 days and then the cells were selected with blasticidin (10 μg/mL, Sigma). Infection and selection efficiency were monitored each day using flow cytometry to measure mCherry (BD Accuri C6). Cells were maintained in spinner flasks in log growth conditions each day by diluting cell concentrations back to a 5×10⁵ cells/mL, with at least 1×10⁸ total cells remaining per replicate such that the lowest maintenance coverage was >18,000× cells per library element. On day 7 post-infection, recruitment was induced by treating the cells with 1000 ng/ml doxycycline (Fisher Scientific) for 2 days, then cells were spun down out of doxycycline and blasticidin and maintained in untreated RPMI media for 4 more days. 2×10⁸ cells were taken for measurements at the day 2 time point. There was no evidence of activation memory at day 4 post-doxycycline removal, as determined by the absence of citrine positive cells by flow cytometry, so no additional time points were collected.

Magnetic separation of reporter cells At each timepoint, cells were spun down at 300×g for 5 minutes and media was aspirated. Cells were then resuspended in the same volume of PBS (Gibco) and the spin down and aspiration was repeated, to wash the cells and remove any IgG from serum. Dynabeads™ M-280 Protein G (ThermoFisher 10003D) were resuspended by vortexing for 30 seconds. 50 mL of blocking buffer was prepared per 2×10⁸ cells by adding 1 gram of biotin-free BSA (Sigma Aldrich) and 200 μl of 0.5 M pH 8.0 EDTA (ThemoFisher 15575020) into DPBS (Gibco), vacuum filtering with a 0.22-μm filter (Millipore), and then kept on ice. 60 μl of beads was prepared for every 1×10⁷ cells, by adding 1 mL of buffer per 200 μl of beads, vortexing for 5 seconds, placing on a magnetic tube rack (Eppendorf), waiting one minute, removing supernatant, and finally removing the beads from the magnet and resuspending in 100-600 μl of blocking buffer per initial 60 μl of beads. For the KRAB DMS only, 30 μl of beads was prepared for every 1×10⁷ cells, in the same way. Beads were added to cells at no more than 1×10⁷ cells per 100 μl of resuspended beads, and then incubated at room temperature while rocking for 30 minutes. For a sample with 2×10⁸ cells, 1.2 mL of beads were used, resuspended in 12 mL of blocking buffer, in a 15 mL Falcon tube and a large magnetic rack. For a sample with <5×10⁷ cells, non-stick Ambion 1.5 mL tubes and a small magnetic rack were used. After incubation, the bead and cell mixture were placed on the magnetic rack for >2 minutes. The unbound supernatant was transferred to a new tube, placed on the magnet again for >2 minutes to remove any remaining beads, and then the supernatant was transferred and saved as the unbound fraction. Then, the beads were resuspended in the same volume of blocking buffer, magnetically separated again, the supernatant was discarded, and the tube with the beads was kept as the bound fraction. The bound fraction was resuspended in blocking buffer or PBS to dilute the cells (the unbound fraction is already dilute). Flow cytometry (BD Accuri) was performed using a small portion of each fraction to estimate the number of cells in each fraction (to ensure library coverage was maintained) and to confirm separation based on citrine reporter levels (the bound fraction should be >90% citrine positive, while the unbound fraction is more variable depending on the initial distribution of reporter levels). Finally, the samples were spun down and the pellets were frozen at −20° C. until genomic DNA extraction.

High-throughput measurement of domain fusion protein expression level The expression level measurements were made in K562-pDY32 cells (with citrine OFF) infected with the 3×FLAG-tagged nuclear Pfam domain library. 1×10⁸ cells per biological replicate were used after 5 days of blasticidin selection (10 μg/mL, Sigma), which was 7 days post-infection. 1×10⁶ control K562-JT039 cells (citrine ON, no lentiviral infection) were spiked into each replicate. Fix Buffer I (BD Biosciences, BDB557870) was preheated to 37° C. for 15 minutes and Permeabilization Buffer III (BD Biosciences, BDB558050) and PBS (Gibco) with 10% FBS (Hyclone) were chilled on ice. The library of cells expressing domains was collected and cell density was counted by flow cytometry (BD Accuri). To fix, cells were resuspended in a volume of Fix Buffer I (BD Biosciences, BDB557870) corresponding to pellet volume, with 20 μl per 1 million cells, at 37° C. for 10-15 minutes. Cells were washed with 1 mL of cold PBS containing 10% FBS, spun down at 500×g for 5 minutes and then supernatant was aspirated. Cells were permeabilized for 30 minutes on ice using cold BD Permeabilization Buffer III (BD Biosciences, BDB558050), with 20 μl per 1 million cells, which was added slowly and mixed by vortexing. Cells were then washed twice in 1 ml PBS+10% FBS, as before, and then supernatant was aspirated. Antibody staining was performed for 1 hour at room temperature, protected from light, using 5 μl/1×10⁶ cells of α-FLAG-Alexa647 (RNDsystems, IC8529R). The cells were washed and resuspended at a concentration of 3×10⁷ cells/ml in PBS+10% FBS. Cells were sorted into two bins based on the level of APC-A fluorescence (Sony SH800S) after gating for mCherry positive viable cells. A small number of unstained control cells was also analyzed on the sorter to confirm staining was above background. The spike-in citrine positive cells were used to assess the background level of staining in cells known to lack the 3×FLAG tag, and the gate for sorting was drawn above that level. After sorting, the cellular coverage ranged from 336-1,295 cells per library element across samples. The sorted cells were spun down at 500×g for 5 minutes and then resuspended in PBS. Genomic DNA extraction was performed following the manufacturer's instructions (QIAgen Blood Maxi kit was used for samples with >1×107 cells, and QIAamp DNA Mini kit with one column per up to 5×10⁶ cells was used for samples with ≤1×10⁷ cells) with one modification: the Proteinase K+AL buffer incubation was performed overnight at 56° C.

Library preparation and sequencing Genomic DNA was extracted using a Blood & Tissue kit (QIAgen) following the manufacturer's instructions with up to 1.25×10⁸ cells per column. DNA was eluted in EB and not AE to avoid subsequence PCR inhibition. The domain sequences were amplified by PCR with primers containing Illumina adapters as extensions. A test PCR was performed using 5 μg of genomic DNA in a 50 μl (half-size) reaction to verify if the PCR conditions would result in a visible band at the expected size for each sample. Then, 12-24×100 μl reactions were set up on ice (in a clean PCR hood to avoid amplifying contaminating DNA), with the number of reactions depending on the amount of genomic DNA available in each experiment. 10 μg of genomic DNA, 0.5 μl of each 100 UM primer, and 50 μl of NEBnext 2× Master Mix (NEB) was used in each reaction. The thermocycling protocol was to preheat the thermocycler to 98° C., then add samples for 3 minutes at 98° C., then 32× cycles of 98° C. for 10 seconds, 63° C. for 30 seconds, 72° C. for 30 seconds, and then a final step of 72° C. for 2 minutes. All subsequent steps were performed outside the PCR hood. The PCR reactions were pooled and ≥140 μl were run on at least three lanes of a 2% TBE gel alongside a 100-bp ladder for at least one hour, the library band around 395 bp was cut out, and DNA was purified using the QIAquick Gel Extraction kit (QIAgen) with a 30 μl elution into non-stick tubes (Ambion), A confirmatory gel was run to verify that small products were removed. These libraries were then quantified with a Qubit HS kit (Thermo Fisher), pooled with 15% PhiX control (Illumina), and sequenced on an Illumina NextSeq with a High output kit using a single end forward read (266 or 300 cycles) and 8 cycle index reads.

Domain sequencing analysis Sequencing reads were demultiplexed using bcl2fastq (Illumina). A Bowtie reference was generated using the designed library sequences with the script ‘makelndices.py’ and reads were aligned with 0 mismatch allowance using the script ‘makeCounts.py’. The enrichments for each domain between OFF and ON (or FLAGhigh and FLAGlow) samples were computed using the script ‘makeRhos.py’. Domains with <5 reads in both samples for a given replicate were dropped from that replicate (assigned 0 counts), whereas domains with <5 reads in one sample would have those reads adjusted to 5 in order to avoid the inflation of enrichment values from low depth. For all of the nuclear domain screens, domains with ≤5 counts in both replicates of a given condition were filtered out of downstream analysis. For the nuclear domain expression screen, well-expressed domains were those with a log 2(FLAGhigh:FLAGlow)≥1 standard deviation above the median of the random controls. For the nuclear Pfam domain repressor screen, hits were domains with log 2(OFF:ON)≥2 standard deviations above the mean of the poorly expressed domains. For the nuclear domain activator screen, hits were domains with log 2(OFF:ON)≤2 standard deviations below the mean of the poorly expressed domains. For the silencer tiling screen, tiles with ≤20 counts in both replicates of a given condition were filtered out and hits were tiles with log 2(OFF:ON)≥2 standard deviations above the mean of the random and DMD tiling controls. Gene ontology analysis enrichments were computed using the PantherDB web tool (www.pantherdb.org). The background sets were all proteins containing domains that were well-expressed and measured in the experiment after count filters were applied. P-values for statistical significance were calculated using Fisher's exact test, the False Discovery Rate (FDR) was computed, and only the most significant results, all with FDR<10%, were shown.

Western blot and co-immunoprecipitation Cells transduced with a lentiviral vector containing an rTetR-fusion-T2A-mCherry-BSD were selected with blasticidin (10 μg/mL) were selected until mCherry was >80%. Cells were lysed in lysis buffer (1% Triton X-100, 150 mM NaCl, 50 mM Tris pH 7.5, 1 mM EDTA, Protease inhibitor cocktail). Protein amounts were quantified using the DC Protein Assay kit (Bio-Rad). Equal amounts were loaded onto a gel and transferred to a nitrocellulose or PVDF membrane. Membrane was probed using GATA1 antibody (1:1000, rabbit, Cell Signaling Technologies cat no. 3535S) and GAPDH antibody (1:2000, mouse, ThermoFisher cat no. AM4300) or FLAG M2 monoclonal antibody (1:1000, mouse, Sigma-Aldrich, catalog number F1804) and Histone 3 antibody (1:1000, mouse, Abcam cat no. AB1791) as primary antibodies. Donkey anti-rabbit IRDye 680 LT and goat anti-mouse IRDye 800CW (1:20,000 dilution, LI-COR Biosciences, cat nos. 926-68023 and 926-32210, respectively) or Goat anti-mouse IRDye 680 RD and goat anti-rabbit IRDye 800CW (1:20,000 dilution, LI-COR Biosciences, cat nos. 926-68070 and 926-32211, respectively) were used as secondary antibodies, respectively

Blots were imaged on a LiCor Odyssey CLx. Band intensities were quantified using ImageJ.

Individual repressor recruitment assays Individual effector domains were cloned as fusions with rTetR or rTetR(SE-G72P) with or without a 3×FLAG tag (see figure legends), upstream of a T2A-mCherry-BSD marker using GoldenGate cloning into backbones pJT050 or pJT126. K562-pJT039-pEF-citrine reporter cells were then transduced with this lentiviral vector and, 3 days later, selected with blasticidin (10 μg/mL) until >80% of the cells were mCherry positive (6-7 days). Cells were split into separate wells of a 24-well plate and either treated with doxycycline (Fisher Scientific) or left untreated. After 5 days of treatment, doxycycline was removed by spinning down the cells, replacing media with DPBS (Gibco) to dilute any remaining doxycycline, and then spinning down the cells again and transferring them to fresh media. Timepoints were measured every 2-3 days by flow cytometry analysis of >7,000 cells (either a BD Accuri C6 or Beckman Coulter CytoFLEX). Data was analyzed using Cytoflow and custom Python scripts. Events were gated for viability and for mCherry as a delivery marker. To compute a fraction of OFF cells during doxycycline treatment, a 2 component Gaussian mixture model was fitted to the untreated rTetR-only negative control cells which fits both the ON peak and the subpopulation of background-silenced OFF cells, and then set a threshold that was 2 standard deviations below the mean of the ON peak in order to label cells that have silenced as OFF. Using the time-matched untreated control, the background normalized percentage of cells was calculated Cells_{OFF,normalized}=Cells_{OFF,+dox}/(1−Cells_{OFF,untreated}). Two independently transduced biological replicates were used. A gene silencing model, consisting of the increasing form of the exponential decay (e.g., exponential decay subtracted from 1) during the doxycycline treatment phase and an exponential decay during the doxycycline removal phase with additional parameters for lag times before silencing and reactivation initiate, was fit to the normalized data using SciPy.

Individual activator recruitment assays Domains were cloned as a fusion with rTetR(SE-G72P) upstream of a T2A-mCherry-BSD marker, using GoldenGate cloning in the backbone pJT126. K562 pDY32 minCMV citrine reporter cells were then transduced with each lentiviral vector and, 3 days later, selected with blasticidin (10 μg/mL) until >80% of the cells were mCherry positive (6-7 days). Cells were split into separate wells of a 24-well plate and either treated with doxycycline or left untreated. Timepoints were measured by flow cytometry analysis of >15,000 cells (Biorad ZE5). To compute a fraction of ON cells during doxycycline treatment, a Gaussian model was fitted to the untreated rTetR-only negative control cells which fits the OFF peak, and then set a threshold that was 2 standard deviations above the mean of the OFF peak in order to label cells that have activated as ON. Two independently transduced biological replicates were used.

Flow cytometry for FLAG-tagged protein levels Staining of FLAG-tagged fusion protein levels was performed. Specifically, K562 cells were transduced with lentivirus to express the fusion proteins, selected with blasticidin, and then were fixed with Fix Buffer I (BD Biosciences) for 15 minutes at 37° C. Cells were washed with cold PBS with 10% FBS once and then permeabilized on ice for 30 min using Perm Buffer III (BD Biosciences). Cells were washed twice and then stained with anti-FLAG (XX) for 1 hour at 4° C. After a final round of washing, flow cytometry was performed using a CytoFLEX (Beckman Coulter) flow cytometer. The data was analyzed with CytoFlow by gating the cells on mCherry expression and then plot the FLAG-tagged protein level in mCherry+ and non-transduced cells. This approach controls for variability in staining efficiency as the two cell groups are mixed within the same sample.

Phylogenetic and alignment analyses KRAB and homeodomain sequences were retrieved from Pfam and extended, using surrounding native sequence, to reach 80 AA. Well-expressed domains were selected for alignment. Phylogenetic trees and sequence alignments were obtained using the alignment website Clustal Omega using default parameters (McWilliam et al., 2013; Sievers et al., 2011), and the 52 phylogenetic neighbor-joining tree without distance corrections was built with default parameters in Jalview (Waterhouse et al., 2009). Alignment visualization was performed in Jalview.

Analysis of amino acid residue conservation Protein sequences were submitted to the ConSurf webserver and analyzed using the ConSeq method. Briefly, ConSeq selects up to 150 homologs for a multiple string alignment, by sampling from the list of homologs with 35-95% sequence identity. Then, a phylogenetic tree is re-constructed and conservation is scored using Rate4Site. ConSurf provides normalized scores, so that the average score for all residues is zero, and the standard deviation is one. The conservation scores calculated by ConSurf are a relative measure of evolutionary conservation at each residue in the protein and the lowest score represents the most conserved position in the protein. The uniqueness of the ZNF10 KRAB N-terminal extension was determined by protein BLAST to all human proteins and searching for other zinc finger protein among the BLAST matches (Johnson et al., 2008).

ChIP-seq and ChIP-exo analysis External ChIP datasets were retrieved from multiple sources. ENCODE ChIP-seq data was processed with the uniform processing pipeline of ENCODE (ENCODE Project Consortium et al., 2020), and narrow peaks below IDR threshold 0.05 were retrieved. KRAB ZNF ChIP-exo data from tagged KRAB ZNF overexpression in HEK293 cells and KAP1 ChIP-exo data from H1 hESCs was obtained from GEO accession GSE78099 (Imbeault et al., 2017). Reads were trimmed to a uniform length of 36 basepairs and mapped to the hg38 version of the human genome using Bowtie (version 1.0.1; (Langmead et al., 2009)), allowing for up to 2 mismatches and only retaining unique alignments. Peak were called using MACS2 (version 2.1.0) (Feng et al., 2012) with the following settings: “-g hs-f BAM --keep-dup all --shift -75 --extsize 150 --nomodel”. Browser tracks were generated using Python scripts. For some KRAB ZNFs where ChIP-exo data was not available, ChIP-seq data from tagged KRAB ZNF overexpression in HEK293 cells was obtained from GEO accessions GSE76496 (Schmitges et al., 2016) and GSE52523 (Najafabadi et al., 2015). KRAB ZNF peaks were defined as solo binding sites if no other KRAB ZNF in the dataset had a peak less than 250 basepairs away. ENCODE H3K27ac ChIP-seq datasets for H1 cells were processed with the ENCODE pipeline (ENCODE Project Consortium et al., 2020), narrow peaks were called with MACS2, and peaks below IDR threshold 0.05 were retrieved.

External datasets ChIP-seq and ChIP-exo data for KRAB ZNF, KAP1, and H3K27ac (ENCODE Project Consortium et al., 2020; Imbeault et al., 2017; Najafabadi et al., 2015; Schmitges et al., 2016), KRAB ZNF gene evolutionary age (Imbeault et al., 2017), KRAB ZNF protein co-immunoprecipitation/mass spectrometry data (Helleboid et al., 2019), and CAT assays for KRAB repressor activity (Margolin et al., 1994; Witzgall et al., 1994) were retrieved from previously published studies.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

TABLE 1
Pfam Repressors

| | Pfam Domain | Extended Domain | Extended Domain | |
|---|---|---|---|---|
| Gene | sequence | sequence | DNA sequence | Avg Repr D5 |
| ZFP28 | SEQ ID NO: 1 | SEQ ID NO: 2 | SEQ ID NO: 1177 | 7.89762464 |
| ZN334 | SEQ ID NO: 3 | SEQ ID NO: 4 | SEQ ID NO: 897 | 7.84632686 |
| ZN568 | SEQ ID NO: 5 | SEQ ID NO: 6 | SEQ ID NO: 898 | 7.83993427 |
| ZN37A | SEQ ID NO: 7 | SEQ ID NO: 8 | SEQ ID NO: 899 | 7.81563526 |
| ZN181 | SEQ ID NO: 9 | SEQ ID NO: 10 | SEQ ID NO: 900 | 7.80113461 |
| ZN510 | SEQ ID NO: 11 | SEQ ID NO: 12 | SEQ ID NO: 901 | 7.65619264 |
| ZN862 | SEQ ID NO: 13 | SEQ ID NO: 14 | SEQ ID NO: 902 | 7.64282609 |
| ZN140 | SEQ ID NO: 15 | SEQ ID NO: 16 | SEQ ID NO: 903 | 7.59939471 |
| ZN208 | SEQ ID NO: 17 | SEQ ID NO: 18 | SEQ ID NO: 904 | 7.57602814 |
| ZN248 | SEQ ID NO: 19 | SEQ ID NO: 20 | SEQ ID NO: 905 | 7.53353306 |
| ZN571 | SEQ ID NO: 21 | SEQ ID NO: 22 | SEQ ID NO: 906 | 7.45303805 |
| ZN699 | SEQ ID NO: 23 | SEQ ID NO: 24 | SEQ ID NO: 907 | 7.44633076 |
| ZN726 | SEQ ID NO: 25 | SEQ ID NO: 26 | SEQ ID NO: 908 | 7.44588981 |
| ZIK1 | SEQ ID NO: 27 | SEQ ID NO: 28 | SEQ ID NO: 909 | 7.43302782 |
| ZNF2 | SEQ ID NO: 29 | SEQ ID NO: 30 | SEQ ID NO: 910 | 7.40745859 |
| Z705F | SEQ ID NO: 31 | SEQ ID NO: 32 | SEQ ID NO: 911 | 7.40598629 |
| ZNF14 | SEQ ID NO: 33 | SEQ ID NO: 34 | SEQ ID NO: 912 | 7.3912024 |
| ZN471 | SEQ ID NO: 35 | SEQ ID NO: 36 | SEQ ID NO: 913 | 7.38691832 |
| ZN624 | SEQ ID NO: 37 | SEQ ID NO: 38 | SEQ ID NO: 914 | 7.37615807 |
| ZNF84 | SEQ ID NO: 39 | SEQ ID NO: 40 | SEQ ID NO: 915 | 7.37354184 |
| ZNF7 | SEQ ID NO: 41 | SEQ ID NO: 42 | SEQ ID NO: 916 | 7.35816861 |
| ZN891 | SEQ ID NO: 43 | SEQ ID NO: 44 | SEQ ID NO: 917 | 7.35404032 |
| ZN337 | SEQ ID NO: 45 | SEQ ID NO: 46 | SEQ ID NO: 918 | 7.3403856 |
| Z705G | SEQ ID NO: 47 | SEQ ID NO: 48 | SEQ ID NO: 919 | 7.33888308 |
| ZN529 | SEQ ID NO: 49 | SEQ ID NO: 50 | SEQ ID NO: 920 | 7.33722191 |
| ZN729 | SEQ ID NO: 51 | SEQ ID NO: 52 | SEQ ID NO: 921 | 7.33489189 |
| ZN419 | SEQ ID NO: 53 | SEQ ID NO: 54 | SEQ ID NO: 922 | 7.33241867 |
| Z705A | SEQ ID NO: 55 | SEQ ID NO: 56 | SEQ ID NO: 923 | 7.32024193 |
| ZNF45 | SEQ ID NO: 57 | SEQ ID NO: 58 | SEQ ID NO: 924 | 7.31275735 |
| ZN302 | SEQ ID NO: 59 | SEQ ID NO: 60 | SEQ ID NO: 925 | 7.27433142 |
| ZN486 | SEQ ID NO: 61 | SEQ ID NO: 62 | SEQ ID NO: 926 | 7.27242434 |
| ZN621 | SEQ ID NO: 63 | SEQ ID NO: 64 | SEQ ID NO: 927 | 7.25940008 |
| ZN688 | SEQ ID NO: 65 | SEQ ID NO: 66 | SEQ ID NO: 928 | 7.2566174 |
| ZN33A | SEQ ID NO: 67 | SEQ ID NO: 68 | SEQ ID NO: 929 | 7.23239827 |
| ZN554 | SEQ ID NO: 69 | SEQ ID NO: 70 | SEQ ID NO: 930 | 7.22964061 |
| ZN878 | SEQ ID NO: 71 | SEQ ID NO: 72 | SEQ ID NO: 931 | 7.21922256 |
| ZN772 | SEQ ID NO: 73 | SEQ ID NO: 74 | SEQ ID NO: 932 | 7.1961596 |
| ZN224 | SEQ ID NO: 75 | SEQ ID NO: 76 | SEQ ID NO: 933 | 7.18876477 |
| ZN184 | SEQ ID NO: 77 | SEQ ID NO: 78 | SEQ ID NO: 934 | 7.18783852 |
| ZN544 | SEQ ID NO: 79 | SEQ ID NO: 80 | SEQ ID NO: 935 | 7.18695522 |
| ZNF57 | SEQ ID NO: 81 | SEQ ID NO: 82 | SEQ ID NO: 936 | 7.1854619 |
| ZN283 | SEQ ID NO: 83 | SEQ ID NO: 84 | SEQ ID NO: 937 | 7.16688066 |
| ZN549 | SEQ ID NO: 85 | SEQ ID NO: 86 | SEQ ID NO: 938 | 7.14938492 |
| ZN211 | SEQ ID NO: 87 | SEQ ID NO: 88 | SEQ ID NO: 939 | 7.14721188 |
| ZN506 | SEQ ID NO: 89 | SEQ ID NO: 90 | SEQ ID NO: 940 | 7.1466168 |
| ZN615 | SEQ ID NO: 91 | SEQ ID NO: 92 | SEQ ID NO: 941 | 7.13864847 |
| ZN253 | SEQ ID NO: 93 | SEQ ID NO: 94 | SEQ ID NO: 942 | 7.12597439 |
| ZN226 | SEQ ID NO: 95 | SEQ ID NO: 96 | SEQ ID NO: 943 | 7.12032078 |
| ZN730 | SEQ ID NO: 97 | SEQ ID NO: 98 | SEQ ID NO: 944 | 7.1167303 |
| Z585A | SEQ ID NO: 99 | SEQ ID NO: 100 | SEQ ID NO: 945 | 7.11150182 |
| ZN732 | SEQ ID NO: 101 | SEQ ID NO: 102 | SEQ ID NO: 946 | 7.10058289 |
| ZN681 | SEQ ID NO: 103 | SEQ ID NO: 104 | SEQ ID NO: 947 | 7.09555392 |
| ZN667 | SEQ ID NO: 105 | SEQ ID NO: 106 | SEQ ID NO: 948 | 7.08035538 |
| ZN649 | SEQ ID NO: 107 | SEQ ID NO: 108 | SEQ ID NO: 949 | 7.07364506 |
| ZN470 | SEQ ID NO: 109 | SEQ ID NO: 110 | SEQ ID NO: 950 | 7.07241961 |
| ZN484 | SEQ ID NO: 111 | SEQ ID NO: 112 | SEQ ID NO: 951 | 7.07124789 |
| ZN431 | SEQ ID NO: 113 | SEQ ID NO: 114 | SEQ ID NO: 952 | 7.06946125 |
| ZN382 | SEQ ID NO: 115 | SEQ ID NO: 116 | SEQ ID NO: 953 | 7.06892645 |
| ZN254 | SEQ ID NO: 117 | SEQ ID NO: 118 | SEQ ID NO: 954 | 7.06718937 |
| ZN124 | SEQ ID NO: 119 | SEQ ID NO: 120 | SEQ ID NO: 955 | 7.0598763 |
| ZN607 | SEQ ID NO: 121 | SEQ ID NO: 122 | SEQ ID NO: 956 | 7.05852729 |
| ZN317 | SEQ ID NO: 123 | SEQ ID NO: 124 | SEQ ID NO: 957 | 7.05281313 |
| ZN620 | SEQ ID NO: 125 | SEQ ID NO: 126 | SEQ ID NO: 958 | 7.04082891 |
| ZN141 | SEQ ID NO: 127 | SEQ ID NO: 128 | SEQ ID NO: 959 | 7.03997569 |
| ZN584 | SEQ ID NO: 129 | SEQ ID NO: 130 | SEQ ID NO: 960 | 7.03820051 |
| ZN540 | SEQ ID NO: 131 | SEQ ID NO: 132 | SEQ ID NO: 961 | 7.03581318 |
| ZN75D | SEQ ID NO: 133 | SEQ ID NO: 134 | SEQ ID NO: 962 | 7.02809755 |
| ZN555 | SEQ ID NO: 135 | SEQ ID NO: 136 | SEQ ID NO: 963 | 7.02680391 |
| ZN658 | SEQ ID NO: 137 | SEQ ID NO: 138 | SEQ ID NO: 964 | 7.01857786 |
| ZN684 | SEQ ID NO: 139 | SEQ ID NO: 140 | SEQ ID NO: 965 | 7.01522838 |
| RBAK | SEQ ID NO: 141 | SEQ ID NO: 142 | SEQ ID NO: 966 | 7.01040328 |
| ZN829 | SEQ ID NO: 143 | SEQ ID NO: 144 | SEQ ID NO: 967 | 7.0012394 |
| ZN582 | SEQ ID NO: 145 | SEQ ID NO: 146 | SEQ ID NO: 968 | 6.98988925 |
| ZN112 | SEQ ID NO: 147 | SEQ ID NO: 148 | SEQ ID NO: 969 | 6.98982538 |
| ZN716 | SEQ ID NO: 149 | SEQ ID NO: 150 | SEQ ID NO: 970 | 6.98744382 |
| HKR1 | SEQ ID NO: 151 | SEQ ID NO: 152 | SEQ ID NO: 971 | 6.98664414 |
| ZN350 | SEQ ID NO: 153 | SEQ ID NO: 154 | SEQ ID NO: 972 | 6.98636848 |
| ZN480 | SEQ ID NO: 155 | SEQ ID NO: 156 | SEQ ID NO: 973 | 6.98462693 |
| ZN416 | SEQ ID NO: 157 | SEQ ID NO: 158 | SEQ ID NO: 974 | 6.97472813 |
| ZNF92 | SEQ ID NO: 159 | SEQ ID NO: 160 | SEQ ID NO: 975 | 6.97138149 |
| ZN100 | SEQ ID NO: 161 | SEQ ID NO: 162 | SEQ ID NO: 976 | 6.9692141 |
| ZN736 | SEQ ID NO: 163 | SEQ ID NO: 164 | SEQ ID NO: 977 | 6.95843452 |
| ZNF74 | SEQ ID NO: 165 | SEQ ID NO: 166 | SEQ ID NO: 978 | 6.95809395 |
| CBX1 | SEQ ID NO: 167 | SEQ ID NO: 168 | SEQ ID NO: 979 | 6.95269512 |
| ZN443 | SEQ ID NO: 169 | SEQ ID NO: 170 | SEQ ID NO: 980 | 6.94561303 |
| ZN195 | SEQ ID NO: 171 | SEQ ID NO: 172 | SEQ ID NO: 981 | 6.9432522 |
| ZN530 | SEQ ID NO: 173 | SEQ ID NO: 174 | SEQ ID NO: 982 | 6.94292737 |
| ZN782 | SEQ ID NO: 175 | SEQ ID NO: 176 | SEQ ID NO: 983 | 6.94217051 |
| ZN791 | SEQ ID NO: 177 | SEQ ID NO: 178 | SEQ ID NO: 984 | 6.93320479 |
| ZN331 | SEQ ID NO: 179 | SEQ ID NO: 180 | SEQ ID NO: 985 | 6.92979428 |
| Z354C | SEQ ID NO: 181 | SEQ ID NO: 182 | SEQ ID NO: 986 | 6.92855271 |
| ZN157 | SEQ ID NO: 183 | SEQ ID NO: 184 | SEQ ID NO: 987 | 6.92764017 |
| ZN727 | SEQ ID NO: 185 | SEQ ID NO: 186 | SEQ ID NO: 988 | 6.9257026 |
| ZN550 | SEQ ID NO: 187 | SEQ ID NO: 188 | SEQ ID NO: 989 | 6.92403295 |
| ZN793 | SEQ ID NO: 189 | SEQ ID NO: 190 | SEQ ID NO: 990 | 6.92326085 |
| ZN235 | SEQ ID NO: 191 | SEQ ID NO: 192 | SEQ ID NO: 991 | 6.91826902 |
| ZNF8 | SEQ ID NO: 193 | SEQ ID NO: 194 | SEQ ID NO: 992 | 6.91722882 |
| ZN724 | SEQ ID NO: 195 | SEQ ID NO: 196 | SEQ ID NO: 993 | 6.89904065 |
| ZN573 | SEQ ID NO: 197 | SEQ ID NO: 198 | SEQ ID NO: 994 | 6.89366942 |
| ZN577 | SEQ ID NO: 199 | SEQ ID NO: 200 | SEQ ID NO: 995 | 6.89093009 |
| ZN789 | SEQ ID NO: 201 | SEQ ID NO: 202 | SEQ ID NO: 996 | 6.88877268 |
| ZN718 | SEQ ID NO: 203 | SEQ ID NO: 204 | SEQ ID NO: 997 | 6.87598723 |
| ZN300 | SEQ ID NO: 205 | SEQ ID NO: 206 | SEQ ID NO: 998 | 6.87019452 |
| ZN383 | SEQ ID NO: 207 | SEQ ID NO: 208 | SEQ ID NO: 999 | 6.86203801 |
| ZN429 | SEQ ID NO: 209 | SEQ ID NO: 210 | SEQ ID NO: 1000 | 6.85768103 |
| ZN677 | SEQ ID NO: 211 | SEQ ID NO: 212 | SEQ ID NO: 1001 | 6.85440091 |
| ZN850 | SEQ ID NO: 213 | SEQ ID NO: 214 | SEQ ID NO: 1002 | 6.85293565 |
| ZN454 | SEQ ID NO: 215 | SEQ ID NO: 216 | SEQ ID NO: 1003 | 6.8342036 |
| ZN257 | SEQ ID NO: 217 | SEQ ID NO: 218 | SEQ ID NO: 1004 | 6.83044 |
| ZN264 | SEQ ID NO: 219 | SEQ ID NO: 220 | SEQ ID NO: 1005 | 6.82889596 |
| ZFP82 | SEQ ID NO: 221 | SEQ ID NO: 222 | SEQ ID NO: 1006 | 6.82733193 |
| ZFP14 | SEQ ID NO: 223 | SEQ ID NO: 224 | SEQ ID NO: 1007 | 6.81312035 |
| ZN485 | SEQ ID NO: 225 | SEQ ID NO: 226 | SEQ ID NO: 1008 | 6.81172703 |
| ZN737 | SEQ ID NO: 227 | SEQ ID NO: 228 | SEQ ID NO: 1009 | 6.80882457 |
| ZNF44 | SEQ ID NO: 229 | SEQ ID NO: 230 | SEQ ID NO: 1010 | 6.80503304 |
| ZN596 | SEQ ID NO: 231 | SEQ ID NO: 232 | SEQ ID NO: 1011 | 6.80500309 |
| ZN565 | SEQ ID NO: 233 | SEQ ID NO: 234 | SEQ ID NO: 1012 | 6.80375161 |
| ZN543 | SEQ ID NO: 235 | SEQ ID NO: 236 | SEQ ID NO: 1013 | 6.79786357 |
| ZFP69 | SEQ ID NO: 237 | SEQ ID NO: 238 | SEQ ID NO: 1014 | 6.79374304 |
| SUMO1 | SEQ ID NO: 239 | SEQ ID NO: 240 | SEQ ID NO: 1015 | 6.77750481 |
| ZNF12 | SEQ ID NO: 241 | SEQ ID NO: 242 | SEQ ID NO: 1016 | 6.77648818 |
| ZN169 | SEQ ID NO: 243 | SEQ ID NO: 244 | SEQ ID NO: 1017 | 6.77498642 |
| ZN433 | SEQ ID NO: 245 | SEQ ID NO: 246 | SEQ ID NO: 1018 | 6.77303438 |
| SUMO3 | SEQ ID NO: 247 | SEQ ID NO: 248 | SEQ ID NO: 1019 | 6.76493545 |
| ZNF98 | SEQ ID NO: 249 | SEQ ID NO: 250 | SEQ ID NO: 1020 | 6.76469777 |
| ZN175 | SEQ ID NO: 251 | SEQ ID NO: 252 | SEQ ID NO: 1021 | 6.76307142 |
| ZN347 | SEQ ID NO: 253 | SEQ ID NO: 254 | SEQ ID NO: 1022 | 6.75405678 |
| ZNF25 | SEQ ID NO: 255 | SEQ ID NO: 256 | SEQ ID NO: 1023 | 6.75008459 |
| ZN519 | SEQ ID NO: 257 | SEQ ID NO: 258 | SEQ ID NO: 1024 | 6.74815071 |
| Z585B | SEQ ID NO: 259 | SEQ ID NO: 260 | SEQ ID NO: 1025 | 6.74700322 |
| ZIM3 | SEQ ID NO: 261 | SEQ ID NO: 262 | SEQ ID NO: 1026 | 6.74462278 |
| ZN517 | SEQ ID NO: 263 | SEQ ID NO: 264 | SEQ ID NO: 1027 | 6.71923079 |
| ZN846 | SEQ ID NO: 265 | SEQ ID NO: 266 | SEQ ID NO: 1028 | 6.70970056 |
| ZN230 | SEQ ID NO: 267 | SEQ ID NO: 268 | SEQ ID NO: 1029 | 6.70246908 |
| ZNF66 | SEQ ID NO: 269 | SEQ ID NO: 270 | SEQ ID NO: 1030 | 6.69981008 |
| ZFP1 | SEQ ID NO: 271 | SEQ ID NO: 272 | SEQ ID NO: 1031 | 6.69334133 |
| ZN713 | SEQ ID NO: 273 | SEQ ID NO: 274 | SEQ ID NO: 1032 | 6.68245851 |
| ZN816 | SEQ ID NO: 275 | SEQ ID NO: 276 | SEQ ID NO: 1033 | 6.67677315 |
| ZN426 | SEQ ID NO: 277 | SEQ ID NO: 278 | SEQ ID NO: 1034 | 6.67185066 |
| ZN701 | SEQ ID NO: 279 | SEQ ID NO: 280 | SEQ ID NO: 1035 | 6.66820921 |
| ZN674 | SEQ ID NO: 281 | SEQ ID NO: 282 | SEQ ID NO: 1036 | 6.6636553 |
| ZN627 | SEQ ID NO: 283 | SEQ ID NO: 284 | SEQ ID NO: 1037 | 6.66232669 |
| ZNF20 | SEQ ID NO: 285 | SEQ ID NO: 286 | SEQ ID NO: 1038 | 6.65839711 |
| Z587B | SEQ ID NO: 287 | SEQ ID NO: 288 | SEQ ID NO: 1039 | 6.63154785 |
| ZN316 | SEQ ID NO: 289 | SEQ ID NO: 290 | SEQ ID NO: 1040 | 6.62746569 |
| ZN233 | SEQ ID NO: 291 | SEQ ID NO: 292 | SEQ ID NO: 1041 | 6.62252575 |
| ZN611 | SEQ ID NO: 293 | SEQ ID NO: 294 | SEQ ID NO: 1042 | 6.61854262 |
| ZN556 | SEQ ID NO: 295 | SEQ ID NO: 296 | SEQ ID NO: 1043 | 6.61519705 |
| ZN234 | SEQ ID NO: 297 | SEQ ID NO: 298 | SEQ ID NO: 1044 | 6.60158035 |
| ZN560 | SEQ ID NO: 299 | SEQ ID NO: 300 | SEQ ID NO: 1045 | 6.60066711 |
| ZNF77 | SEQ ID NO: 301 | SEQ ID NO: 302 | SEQ ID NO: 1046 | 6.58987943 |
| ZN682 | SEQ ID NO: 303 | SEQ ID NO: 304 | SEQ ID NO: 1047 | 6.58030961 |
| ZN614 | SEQ ID NO: 305 | SEQ ID NO: 306 | SEQ ID NO: 1048 | 6.57723831 |
| ZN785 | SEQ ID NO: 307 | SEQ ID NO: 308 | SEQ ID NO: 1049 | 6.56301724 |
| ZN445 | SEQ ID NO: 309 | SEQ ID NO: 310 | SEQ ID NO: 1050 | 6.54429484 |
| ZFP30 | SEQ ID NO: 311 | SEQ ID NO: 312 | SEQ ID NO: 1051 | 6.54105426 |
| ZN225 | SEQ ID NO: 313 | SEQ ID NO: 314 | SEQ ID NO: 1052 | 6.53858149 |
| ZN551 | SEQ ID NO: 315 | SEQ ID NO: 316 | SEQ ID NO: 1053 | 6.53471613 |
| ZN610 | SEQ ID NO: 317 | SEQ ID NO: 318 | SEQ ID NO: 1054 | 6.53304307 |
| ZN528 | SEQ ID NO: 319 | SEQ ID NO: 320 | SEQ ID NO: 1055 | 6.5320662 |
| ZN284 | SEQ ID NO: 321 | SEQ ID NO: 322 | SEQ ID NO: 1056 | 6.52062588 |
| ZN418 | SEQ ID NO: 323 | SEQ ID NO: 324 | SEQ ID NO: 1057 | 6.51925026 |
| MPP8 | SEQ ID NO: 325 | SEQ ID NO: 326 | SEQ ID NO: 1058 | 6.51334634 |
| ZN490 | SEQ ID NO: 327 | SEQ ID NO: 328 | SEQ ID NO: 1059 | 6.51148602 |
| ZN805 | SEQ ID NO: 329 | SEQ ID NO: 330 | SEQ ID NO: 1060 | 6.50974725 |
| Z780B | SEQ ID NO: 331 | SEQ ID NO: 332 | SEQ ID NO: 1061 | 6.50607891 |
| ZN763 | SEQ ID NO: 333 | SEQ ID NO: 334 | SEQ ID NO: 1062 | 6.49330748 |
| ZN285 | SEQ ID NO: 335 | SEQ ID NO: 336 | SEQ ID NO: 1063 | 6.48639829 |
| ZNF85 | SEQ ID NO: 337 | SEQ ID NO: 338 | SEQ ID NO: 1064 | 6.48512557 |
| ZN223 | SEQ ID NO: 339 | SEQ ID NO: 340 | SEQ ID NO: 1065 | 6.48230966 |
| ZNF90 | SEQ ID NO: 341 | SEQ ID NO: 342 | SEQ ID NO: 1066 | 6.47855756 |
| ZN557 | SEQ ID NO: 343 | SEQ ID NO: 344 | SEQ ID NO: 1067 | 6.47397343 |
| ZN425 | SEQ ID NO: 345 | SEQ ID NO: 346 | SEQ ID NO: 1068 | 6.47320582 |
| ZN229 | SEQ ID NO: 347 | SEQ ID NO: 348 | SEQ ID NO: 1069 | 6.47139743 |
| ZN606 | SEQ ID NO: 349 | SEQ ID NO: 350 | SEQ ID NO: 1070 | 6.46489693 |
| ZN155 | SEQ ID NO: 351 | SEQ ID NO: 352 | SEQ ID NO: 1071 | 6.45744473 |
| ZN222 | SEQ ID NO: 353 | SEQ ID NO: 354 | SEQ ID NO: 1072 | 6.45544011 |
| ZN442 | SEQ ID NO: 355 | SEQ ID NO: 356 | SEQ ID NO: 1073 | 6.44268455 |
| ZNF91 | SEQ ID NO: 357 | SEQ ID NO: 358 | SEQ ID NO: 1074 | 6.44174437 |
| ZN135 | SEQ ID NO: 359 | SEQ ID NO: 360 | SEQ ID NO: 1075 | 6.44116741 |
| ZN778 | SEQ ID NO: 361 | SEQ ID NO: 362 | SEQ ID NO: 1076 | 6.43548986 |
| RYBP | SEQ ID NO: 363 | SEQ ID NO: 364 | SEQ ID NO: 1077 | 6.42734946 |
| ZN534 | SEQ ID NO: 365 | SEQ ID NO: 366 | SEQ ID NO: 1078 | 6.42731382 |
| ZN586 | SEQ ID NO: 367 | SEQ ID NO: 368 | SEQ ID NO: 1079 | 6.41123861 |
| ZN567 | SEQ ID NO: 369 | SEQ ID NO: 370 | SEQ ID NO: 1080 | 6.40288995 |
| ZN440 | SEQ ID NO: 371 | SEQ ID NO: 372 | SEQ ID NO: 1081 | 6.40187146 |
| ZN583 | SEQ ID NO: 373 | SEQ ID NO: 374 | SEQ ID NO: 1082 | 6.39776145 |
| ZN441 | SEQ ID NO: 375 | SEQ ID NO: 376 | SEQ ID NO: 1083 | 6.38715626 |
| ZNF43 | SEQ ID NO: 377 | SEQ ID NO: 378 | SEQ ID NO: 1084 | 6.38246564 |
| CBX5 | SEQ ID NO: 379 | SEQ ID NO: 380 | SEQ ID NO: 1085 | 6.36905016 |
| ZN589 | SEQ ID NO: 381 | SEQ ID NO: 382 | SEQ ID NO: 1086 | 6.36425087 |
| ZNF10 | SEQ ID NO: 383 | SEQ ID NO: 384 | SEQ ID NO: 1087 | 6.36134473 |
| ZN563 | SEQ ID NO: 385 | SEQ ID NO: 386 | SEQ ID NO: 1088 | 6.3562145 |
| ZN561 | SEQ ID NO: 387 | SEQ ID NO: 388 | SEQ ID NO: 1089 | 6.3525504 |
| ZN136 | SEQ ID NO: 389 | SEQ ID NO: 390 | SEQ ID NO: 1090 | 6.35103846 |
| ZN630 | SEQ ID NO: 391 | SEQ ID NO: 392 | SEQ ID NO: 1091 | 6.34648094 |
| ZN527 | SEQ ID NO: 393 | SEQ ID NO: 394 | SEQ ID NO: 1092 | 6.34024936 |
| ZN333 | SEQ ID NO: 395 | SEQ ID NO: 396 | SEQ ID NO: 1093 | 6.33883721 |
| Z324B | SEQ ID NO: 397 | SEQ ID NO: 398 | SEQ ID NO: 1094 | 6.33798774 |
| ZN786 | SEQ ID NO: 399 | SEQ ID NO: 400 | SEQ ID NO: 1095 | 6.31659272 |
| ZN709 | SEQ ID NO: 401 | SEQ ID NO: 402 | SEQ ID NO: 1096 | 6.31480293 |
| ZN792 | SEQ ID NO: 403 | SEQ ID NO: 404 | SEQ ID NO: 1097 | 6.29907418 |
| ZN599 | SEQ ID NO: 405 | SEQ ID NO: 406 | SEQ ID NO: 1098 | 6.29676005 |
| ZN613 | SEQ ID NO: 407 | SEQ ID NO: 408 | SEQ ID NO: 1099 | 6.28970926 |
| ZF69B | SEQ ID NO: 409 | SEQ ID NO: 410 | SEQ ID NO: 1100 | 6.28648867 |
| ZN799 | SEQ ID NO: 411 | SEQ ID NO: 412 | SEQ ID NO: 1101 | 6.28580406 |
| ZN569 | SEQ ID NO: 413 | SEQ ID NO: 414 | SEQ ID NO: 1102 | 6.28572758 |
| ZN564 | SEQ ID NO: 415 | SEQ ID NO: 416 | SEQ ID NO: 1103 | 6.28268424 |
| ZN546 | SEQ ID NO: 417 | SEQ ID NO: 418 | SEQ ID NO: 1104 | 6.27774396 |
| ZFP92 | SEQ ID NO: 419 | SEQ ID NO: 420 | SEQ ID NO: 1105 | 6.273403 |
| YAF2 | SEQ ID NO: 421 | SEQ ID NO: 422 | SEQ ID NO: 1106 | 6.25768891 |
| ZN723 | SEQ ID NO: 423 | SEQ ID NO: 424 | SEQ ID NO: 1107 | 6.25047465 |
| ZNF34 | SEQ ID NO: 425 | SEQ ID NO: 426 | SEQ ID NO: 1108 | 6.23513709 |
| ZN439 | SEQ ID NO: 427 | SEQ ID NO: 428 | SEQ ID NO: 1109 | 6.22934428 |
| ZFP57 | SEQ ID NO: 429 | SEQ ID NO: 430 | SEQ ID NO: 1110 | 6.2234497 |
| ZNF19 | SEQ ID NO: 431 | SEQ ID NO: 432 | SEQ ID NO: 1111 | 6.21632085 |
| ZN404 | SEQ ID NO: 433 | SEQ ID NO: 434 | SEQ ID NO: 1112 | 6.20126205 |
| ZN274 | SEQ ID NO: 435 | SEQ ID NO: 436 | SEQ ID NO: 1113 | 6.19652061 |
| CBX3 | SEQ ID NO: 437 | SEQ ID NO: 438 | SEQ ID NO: 1114 | 6.19641648 |
| ZNF30 | SEQ ID NO: 439 | SEQ ID NO: 440 | SEQ ID NO: 1115 | 6.19503476 |
| ZN250 | SEQ ID NO: 441 | SEQ ID NO: 442 | SEQ ID NO: 1116 | 6.17058573 |
| ZN570 | SEQ ID NO: 443 | SEQ ID NO: 444 | SEQ ID NO: 1117 | 6.16932644 |
| ZN675 | SEQ ID NO: 445 | SEQ ID NO: 446 | SEQ ID NO: 1118 | 6.15995772 |
| ZN695 | SEQ ID NO: 447 | SEQ ID NO: 448 | SEQ ID NO: 1119 | 6.15609798 |
| ZN548 | SEQ ID NO: 449 | SEQ ID NO: 450 | SEQ ID NO: 1120 | 6.14238152 |
| ZN227 | SEQ ID NO: 451 | SEQ ID NO: 452 | SEQ ID NO: 1121 | 6.13508917 |
| ZN132 | SEQ ID NO: 453 | SEQ ID NO: 454 | SEQ ID NO: 1122 | 6.13316124 |
| ZN738 | SEQ ID NO: 455 | SEQ ID NO: 456 | SEQ ID NO: 1123 | 6.12742065 |
| ZN420 | SEQ ID NO: 457 | SEQ ID NO: 458 | SEQ ID NO: 1124 | 6.1074573 |
| ZN514 | SEQ ID NO: 459 | SEQ ID NO: 460 | SEQ ID NO: 1125 | 6.10685195 |
| ZN626 | SEQ ID NO: 461 | SEQ ID NO: 462 | SEQ ID NO: 1126 | 6.10541852 |
| ZN806 | SEQ ID NO: 463 | SEQ ID NO: 464 | SEQ ID NO: 1127 | 6.09805184 |
| ZN559 | SEQ ID NO: 465 | SEQ ID NO: 466 | SEQ ID NO: 1128 | 6.09618421 |
| ZN460 | SEQ ID NO: 467 | SEQ ID NO: 468 | SEQ ID NO: 1129 | 6.08494207 |
| ZN268 | SEQ ID NO: 469 | SEQ ID NO: 470 | SEQ ID NO: 1130 | 6.040812 |
| ZN304 | SEQ ID NO: 471 | SEQ ID NO: 472 | SEQ ID NO: 1131 | 6.03800144 |
| ZIM2 | SEQ ID NO: 473 | SEQ ID NO: 474 | SEQ ID NO: 1132 | 6.03746453 |
| ZN605 | SEQ ID NO: 475 | SEQ ID NO: 476 | SEQ ID NO: 1133 | 6.01346476 |
| ZN844 | SEQ ID NO: 477 | SEQ ID NO: 478 | SEQ ID NO: 1134 | 5.98806163 |
| SUMO5 | SEQ ID NO: 479 | SEQ ID NO: 480 | SEQ ID NO: 1135 | 5.96583945 |
| ZN101 | SEQ ID NO: 481 | SEQ ID NO: 482 | SEQ ID NO: 1136 | 5.90648424 |
| ZN783 | SEQ ID NO: 483 | SEQ ID NO: 484 | SEQ ID NO: 1137 | 5.87160607 |
| ZN417 | SEQ ID NO: 485 | SEQ ID NO: 486 | SEQ ID NO: 1138 | 5.85910987 |
| ZN182 | SEQ ID NO: 487 | SEQ ID NO: 488 | SEQ ID NO: 1139 | 5.80251318 |
| ZN823 | SEQ ID NO: 489 | SEQ ID NO: 490 | SEQ ID NO: 1140 | 5.75436578 |
| ZN177 | SEQ ID NO: 491 | SEQ ID NO: 492 | SEQ ID NO: 1141 | 5.66150299 |
| ZN197 | SEQ ID NO: 493 | SEQ ID NO: 494 | SEQ ID NO: 1142 | 5.65816459 |
| ZN717 | SEQ ID NO: 495 | SEQ ID NO: 496 | SEQ ID NO: 1143 | 5.64802359 |
| ZN669 | SEQ ID NO: 497 | SEQ ID NO: 498 | SEQ ID NO: 1144 | 5.58623836 |
| ZN256 | SEQ ID NO: 499 | SEQ ID NO: 500 | SEQ ID NO: 1145 | 5.57864488 |
| ZN251 | SEQ ID NO: 501 | SEQ ID NO: 502 | SEQ ID NO: 1146 | 5.54680119 |
| CBX4 | SEQ ID NO: 503 | SEQ ID NO: 504 | SEQ ID NO: 1147 | 5.47206529 |
| PCGF2 | SEQ ID NO: 505 | SEQ ID NO: 506 | SEQ ID NO: 1148 | 5.41711547 |
| CDY2 | SEQ ID NO: 507 | SEQ ID NO: 508 | SEQ ID NO: 1149 | 5.20865573 |
| CDYL2 | SEQ ID NO: 509 | SEQ ID NO: 510 | SEQ ID NO: 1150 | 5.17777542 |
| ZN287 | SEQ ID NO: 511 | SEQ ID NO: 512 | SEQ ID NO: 1151 | 5.15786106 |
| HERC2 | SEQ ID NO: 513 | SEQ ID NO: 514 | SEQ ID NO: 1152 | 5.12990133 |
| ZN562 | SEQ ID NO: 515 | SEQ ID NO: 516 | SEQ ID NO: 1153 | 5.08331004 |
| ZN461 | SEQ ID NO: 517 | SEQ ID NO: 518 | SEQ ID NO: 1154 | 5.05101639 |
| Z324A | SEQ ID NO: 519 | SEQ ID NO: 520 | SEQ ID NO: 1155 | 5.01043067 |
| ZN766 | SEQ ID NO: 521 | SEQ ID NO: 522 | SEQ ID NO: 1156 | 4.9926318 |
| ID2 | SEQ ID NO: 523 | SEQ ID NO: 524 | SEQ ID NO: 1157 | 4.86972562 |
| TOX | SEQ ID NO: 525 | SEQ ID NO: 526 | SEQ ID NO: 1158 | 4.84737013 |
| ZN274 | SEQ ID NO: 527 | SEQ ID NO: 528 | SEQ ID NO: 1159 | 4.82395142 |
| ZN75C | SEQ ID NO: 529 | SEQ ID NO: 530 | SEQ ID NO: 1160 | 4.81809368 |
| SCMH1 | SEQ ID NO: 531 | SEQ ID NO: 532 | SEQ ID NO: 1161 | 4.79639316 |
| ZN560 | SEQ ID NO: 533 | SEQ ID NO: 534 | SEQ ID NO: 1162 | 4.77465441 |
| SCML4 | SEQ ID NO: 535 | SEQ ID NO: 536 | SEQ ID NO: 1163 | 4.74079704 |
| ZN214 | SEQ ID NO: 537 | SEQ ID NO: 538 | SEQ ID NO: 1164 | 4.72989473 |
| CBX7 | SEQ ID NO: 539 | SEQ ID NO: 540 | SEQ ID NO: 1165 | 4.70199486 |
| ID1 | SEQ ID NO: 541 | SEQ ID NO: 542 | SEQ ID NO: 1166 | 4.66128008 |
| CREM | SEQ ID NO: 543 | SEQ ID NO: 544 | SEQ ID NO: 1167 | 4.58757659 |
| FER3L | SEQ ID NO: 545 | SEQ ID NO: 546 | SEQ ID NO: 1168 | 4.55608825 |
| SCX | SEQ ID NO: 547 | SEQ ID NO: 548 | SEQ ID NO: 1169 | 4.38664628 |
| ASCL1 | SEQ ID NO: 549 | SEQ ID NO: 550 | SEQ ID NO: 1170 | 4.23952129 |
| ZN764 | SEQ ID NO: 551 | SEQ ID NO: 552 | SEQ ID NO: 1171 | 4.16413141 |
| SCML2 | SEQ ID NO: 553 | SEQ ID NO: 554 | SEQ ID NO: 1172 | 4.16119992 |
| ASCL5 | SEQ ID NO: 555 | SEQ ID NO: 556 | SEQ ID NO: 1173 | 4.14708139 |
| TWST1 | SEQ ID NO: 557 | SEQ ID NO: 558 | SEQ ID NO: 1174 | 4.09571741 |
| ZN319 | SEQ ID NO: 559 | SEQ ID NO: 560 | SEQ ID NO: 1175 | 4.08013835 |
| ZN749 | SEQ ID NO: 561 | SEQ ID NO: 562 | SEQ ID NO: 1176 | 4.06508464 |

TABLE 2
Pfam Activators

| | Pfam Domain | Extended Domain | Extended Domain | |
|---|---|---|---|---|
| Gene | sequence | sequence | DNA sequence | Avg Activation |
| ZN473 | SEQ ID NO: 563 | SEQ ID NO: 564 | SEQ ID NO: 1178 | −8.6232004 |
| FOXO3 | SEQ ID NO: 565 | SEQ ID NO: 566 | SEQ ID NO: 1179 | −8.3891724 |
| FOXO1 | SEQ ID NO: 567 | SEQ ID NO: 568 | SEQ ID NO: 1180 | −8.3703632 |
| MYBA | SEQ ID NO: 569 | SEQ ID NO: 570 | SEQ ID NO: 1181 | −8.2096102 |
| MYB | SEQ ID NO: 571 | SEQ ID NO: 572 | SEQ ID NO: 1182 | −7.2112528 |
| NCOA2 | SEQ ID NO: 573 | SEQ ID NO: 574 | SEQ ID NO: 1183 | −7.1119077 |
| SMCA2 | SEQ ID NO: 575 | SEQ ID NO: 576 | SEQ ID NO: 1184 | −6.7916451 |
| KIBRA | SEQ ID NO: 577 | SEQ ID NO: 578 | SEQ ID NO: 1185 | −6.707792 |
| NCOA3 | SEQ ID NO: 579 | SEQ ID NO: 580 | SEQ ID NO: 1186 | −6.4149356 |
| FOXO6 | SEQ ID NO: 581 | SEQ ID NO: 582 | SEQ ID NO: 1187 | −6.0518896 |
| ZN597 | SEQ ID NO: 583 | SEQ ID NO: 584 | SEQ ID NO: 1188 | −5.9555177 |
| APBB1 | SEQ ID NO: 585 | SEQ ID NO: 586 | SEQ ID NO: 1189 | −5.8338079 |
| ANM2 | SEQ ID NO: 587 | SEQ ID NO: 588 | SEQ ID NO: 1190 | −5.6456716 |
| MED9 | SEQ ID NO: 589 | SEQ ID NO: 590 | SEQ ID NO: 1191 | −5.5377024 |
| CXXC1 | SEQ ID NO: 591 | SEQ ID NO: 592 | SEQ ID NO: 1192 | −5.4566266 |
| CRTC2 | SEQ ID NO: 593 | SEQ ID NO: 594 | SEQ ID NO: 1193 | −5.293256 |
| NOTC2 | SEQ ID NO: 595 | SEQ ID NO: 596 | SEQ ID NO: 1194 | −5.2584004 |
| CACO1 | SEQ ID NO: 597 | SEQ ID NO: 598 | SEQ ID NO: 1195 | −4.6832738 |
| PYGO1 | SEQ ID NO: 599 | SEQ ID NO: 600 | SEQ ID NO: 1196 | −4.3430928 |
| IKKA | SEQ ID NO: 601 | SEQ ID NO: 602 | SEQ ID NO: 1197 | −4.3328612 |
| APC16 | SEQ ID NO: 603 | SEQ ID NO: 604 | SEQ ID NO: 1198 | −4.1227423 |
| WWP2 | SEQ ID NO: 605 | SEQ ID NO: 606 | SEQ ID NO: 1199 | −4.0489585 |
| RIP | SEQ ID NO: 607 | SEQ ID NO: 608 | SEQ ID NO: 1200 | −3.97129 |
| AF9 | SEQ ID NO: 609 | SEQ ID NO: 610 | SEQ ID NO: 1201 | −3.7419986 |
| ZFP28 | SEQ ID NO: 611 | SEQ ID NO: 612 | SEQ ID NO: 1202 | −3.7291024 |
| WWP1 | SEQ ID NO: 613 | SEQ ID NO: 614 | SEQ ID NO: 1203 | −3.728405 |
| DPY30 | SEQ ID NO: 615 | SEQ ID NO: 616 | SEQ ID NO: 1204 | −3.696281 |
| KS6B2 | SEQ ID NO: 617 | SEQ ID NO: 618 | SEQ ID NO: 1205 | −3.4939583 |
| PYGO2 | SEQ ID NO: 619 | SEQ ID NO: 620 | SEQ ID NO: 1206 | −3.4423787 |
| U2AF4 | SEQ ID NO: 621 | SEQ ID NO: 622 | SEQ ID NO: 1207 | −3.3553928 |
| ITCH | SEQ ID NO: 623 | SEQ ID NO: 624 | SEQ ID NO: 1208 | −3.3366968 |
| ENL | SEQ ID NO: 625 | SEQ ID NO: 626 | SEQ ID NO: 1209 | −3.3117985 |
| STAT2 | SEQ ID NO: 627 | SEQ ID NO: 628 | SEQ ID NO: 1210 | −3.1207026 |
| NOTC1 | SEQ ID NO: 629 | SEQ ID NO: 630 | SEQ ID NO: 1211 | −3.1201108 |
| CRTC3 | SEQ ID NO: 631 | SEQ ID NO: 632 | SEQ ID NO: 1212 | −3.0736492 |
| SAV1 | SEQ ID NO: 633 | SEQ ID NO: 634 | SEQ ID NO: 1213 | −2.9035402 |
| DPF1 | SEQ ID NO: 635 | SEQ ID NO: 636 | SEQ ID NO: 1214 | −2.7433919 |
| ABL1 | SEQ ID NO: 637 | SEQ ID NO: 638 | SEQ ID NO: 1215 | −2.6728209 |
| WBP4 | SEQ ID NO: 639 | SEQ ID NO: 640 | SEQ ID NO: 1216 | −2.6121807 |
| BTK | SEQ ID NO: 641 | SEQ ID NO: 642 | SEQ ID NO: 1217 | −2.5651252 |
| SMRC2 | SEQ ID NO: 643 | SEQ ID NO: 644 | SEQ ID NO: 1218 | −2.4978538 |
| MTA3 | SEQ ID NO: 645 | SEQ ID NO: 646 | SEQ ID NO: 1219 | −2.4098352 |
| WWTR1 | SEQ ID NO: 647 | SEQ ID NO: 648 | SEQ ID NO: 1220 | −2.3989581 |
| EGR3 | SEQ ID NO: 649 | SEQ ID NO: 650 | SEQ ID NO: 1221 | −2.337045 |
| NFIX | SEQ ID NO: 651 | SEQ ID NO: 652 | SEQ ID NO: 1222 | −2.289111 |
| KPCI | SEQ ID NO: 653 | SEQ ID NO: 654 | SEQ ID NO: 1223 | −2.2334296 |
| LMBL1 | SEQ ID NO: 655 | SEQ ID NO: 656 | SEQ ID NO: 1224 | −2.2075416 |
| NOTC1 | SEQ ID NO: 657 | SEQ ID NO: 658 | SEQ ID NO: 1225 | −2.1840238 |
| FIGN | SEQ ID NO: 659 | SEQ ID NO: 660 | SEQ ID NO: 1226 | −2.1805996 |
| IMAS | SEQ ID NO: 661 | SEQ ID NO: 662 | SEQ ID NO: 1227 | −2.15155 |
| ZN496 | SEQ ID NO: 663 | SEQ ID NO: 664 | SEQ ID NO: 1228 | −2.1412028 |

TABLE 3
KRAB Repressor Mutants

| | | | | Norm Avg D13 |
|---|---|---|---|---|
| | Start of | Amino Acid | DNA | (0 = Wild |
| Variant | Mutation | sequence | sequence | Type score) |
| GlutamicAcid; 3; 5 | 5 | SEQ ID NO: 665 | SEQ ID NO: 1229 | 1.28823665 |
| GlutamicAcid; 3; 7 | 7 | SEQ ID NO: 666 | SEQ ID NO: 1230 | 1.14468005 |
| AsparticAcid; 3; 7 | 7 | SEQ ID NO: 667 | SEQ ID NO: 1231 | 1.10622079 |
| Isoleucine; 3; 67 | 67 | SEQ ID NO: 668 | SEQ ID NO: 1232 | 1.08345235 |
| Proline; 3; 6 | 6 | SEQ ID NO: 669 | SEQ ID NO: 1233 | 1.06556067 |
| Asparagine; 1; 1 | 1 | SEQ ID NO: 670 | SEQ ID NO: 1234 | 1.05496491 |
| Proline; 3; 9 | 9 | SEQ ID NO: 671 | SEQ ID NO: 1235 | 1.05426168 |
| GlutamicAcid; 2; 6 | 6 | SEQ ID NO: 672 | SEQ ID NO: 1236 | 1.04111335 |
| Alanine; 2; 7 | 7 | SEQ ID NO: 673 | SEQ ID NO: 1237 | 1.04035858 |
| Valine; 1; 4 | 4 | SEQ ID NO: 674 | SEQ ID NO: 1238 | 0.96430263 |
| Proline; 1; 7 | 7 | SEQ ID NO: 675 | SEQ ID NO: 1239 | 0.96424497 |
| Glycine; 2; 7 | 7 | SEQ ID NO: 676 | SEQ ID NO: 1240 | 0.94837463 |
| Asparagine; 3; 5 | 5 | SEQ ID NO: 677 | SEQ ID NO: 1241 | 0.92066978 |
| Glutamine; 3; 7 | 7 | SEQ ID NO: 678 | SEQ ID NO: 1242 | 0.91183545 |
| Proline; 2; 10 | 10 | SEQ ID NO: 679 | SEQ ID NO: 1243 | 0.89572995 |
| Threonine; 2; 7 | 7 | SEQ ID NO: 680 | SEQ ID NO: 1244 | 0.88884291 |
| GlutamicAcid; 2; 10 | 10 | SEQ ID NO: 681 | SEQ ID NO: 1245 | 0.86791044 |
| AsparticAcid; 2; 7 | 7 | SEQ ID NO: 682 | SEQ ID NO: 1246 | 0.86480685 |
| Glutamine; 3; 6 | 6 | SEQ ID NO: 683 | SEQ ID NO: 1247 | 0.86314843 |
| GlutamicAcid; 3; 4 | 4 | SEQ ID NO: 684 | SEQ ID NO: 1248 | 0.84553985 |
| AsparticAcid; 2; 6 | 6 | SEQ ID NO: 685 | SEQ ID NO: 1249 | 0.84522896 |
| Asparagine; 2; 7 | 7 | SEQ ID NO: 686 | SEQ ID NO: 1250 | 0.84228978 |
| Asparagine; 3; 0 | 0 | SEQ ID NO: 687 | SEQ ID NO: 1251 | 0.83772353 |
| Glycine; 3; 5 | 5 | SEQ ID NO: 688 | SEQ ID NO: 1252 | 0.83010312 |
| AsparticAcid; 3; 6 | 6 | SEQ ID NO: 689 | SEQ ID NO: 1253 | 0.82122205 |
| AsparticAcid; 3; 5 | 5 | SEQ ID NO: 690 | SEQ ID NO: 1254 | 0.81720761 |
| Lysine; 2; 62 | 62 | SEQ ID NO: 691 | SEQ ID NO: 1255 | 0.79372667 |
| AsparticAcid; 1; 7 | 7 | SEQ ID NO: 692 | SEQ ID NO: 1256 | 0.77854784 |
| Lysine; 3; 5 | 5 | SEQ ID NO: 693 | SEQ ID NO: 1257 | 0.76670536 |
| GlutamicAcid; 1; 7 | 7 | SEQ ID NO: 694 | SEQ ID NO: 1258 | 0.76316547 |
| Proline; 2; 7 | 7 | SEQ ID NO: 695 | SEQ ID NO: 1259 | 0.7537889 |
| Proline; 3; 7 | 7 | SEQ ID NO: 696 | SEQ ID NO: 1260 | 0.74610592 |
| AsparticAcid; 2; 8 | 8 | SEQ ID NO: 697 | SEQ ID NO: 1261 | 0.74252065 |
| Asparagine; 3; 7 | 7 | SEQ ID NO: 698 | SEQ ID NO: 1262 | 0.72618378 |
| Methionine; 1; 7 | 7 | SEQ ID NO: 699 | SEQ ID NO: 1263 | 0.71061025 |
| Asparagine; 3; 8 | 8 | SEQ ID NO: 700 | SEQ ID NO: 1264 | 0.70490419 |
| Asparagine; 2; 9 | 9 | SEQ ID NO: 701 | SEQ ID NO: 1265 | 0.7021223 |
| Proline; 2; 6 | 6 | SEQ ID NO: 702 | SEQ ID NO: 1266 | 0.70094498 |
| GlutamicAcid; 3; 6 | 6 | SEQ ID NO: 703 | SEQ ID NO: 1267 | 0.69641957 |
| Alanine; 3; 5 | 5 | SEQ ID NO: 704 | SEQ ID NO: 1268 | 0.69263933 |
| Glutamine; 2; 7 | 7 | SEQ ID NO: 705 | SEQ ID NO: 1269 | 0.67752105 |
| GlutamicAcid; 3; 9 | 9 | SEQ ID NO: 706 | SEQ ID NO: 1270 | 0.66806615 |
| Glutamine; 2; 6 | 6 | SEQ ID NO: 707 | SEQ ID NO: 1271 | 0.65472821 |
| GlutamicAcid; 1; 39 | 39 | SEQ ID NO: 708 | SEQ ID NO: 1272 | 0.65227203 |
| Proline; 1; 10 | 10 | SEQ ID NO: 709 | SEQ ID NO: 1273 | 0.64837531 |
| Threonine; 1; 4 | 4 | SEQ ID NO: 710 | SEQ ID NO: 1274 | 0.64360588 |
| Asparagine; 3; 9 | 9 | SEQ ID NO: 711 | SEQ ID NO: 1275 | 0.63949165 |
| AsparticAcid; 3; 8 | 8 | SEQ ID NO: 712 | SEQ ID NO: 1276 | 0.63564982 |
| Alanine; 2; 6 | 6 | SEQ ID NO: 713 | SEQ ID NO: 1277 | 0.63459967 |
| Glycine; 1; 7 | 7 | SEQ ID NO: 714 | SEQ ID NO: 1278 | 0.6338712 |
| Serine; 1; 7 | 7 | SEQ ID NO: 715 | SEQ ID NO: 1279 | 0.6299573 |
| Serine; 2; 6 | 6 | SEQ ID NO: 716 | SEQ ID NO: 1280 | 0.61708486 |
| AsparticAcid; 2; 3 | 3 | SEQ ID NO: 717 | SEQ ID NO: 1281 | 0.61493283 |
| Histidine; 1; 55 | 55 | SEQ ID NO: 718 | SEQ ID NO: 1282 | 0.59443523 |
| Lysine; 2; 6 | 6 | SEQ ID NO: 719 | SEQ ID NO: 1283 | 0.59389075 |
| GlutamicAcid; 2; 9 | 9 | SEQ ID NO: 720 | SEQ ID NO: 1284 | 0.58357862 |
| Glutamine; 3; 8 | 8 | SEQ ID NO: 721 | SEQ ID NO: 1285 | 0.58274443 |
| Threonine; 1; 11 | 11 | SEQ ID NO: 722 | SEQ ID NO: 1286 | 0.57209112 |
| Asparagine; 1; 7 | 7 | SEQ ID NO: 723 | SEQ ID NO: 1287 | 0.57143202 |
| Glutamine; 3; 5 | 5 | SEQ ID NO: 724 | SEQ ID NO: 1288 | 0.57133084 |
| Proline; 2; 8 | 8 | SEQ ID NO: 725 | SEQ ID NO: 1289 | 0.56714292 |
| GlutamicAcid; 3; 3 | 3 | SEQ ID NO: 726 | SEQ ID NO: 1290 | 0.55531398 |
| Asparagine; 2; 6 | 6 | SEQ ID NO: 727 | SEQ ID NO: 1291 | 0.55430182 |
| AsparticAcid; 1; 11 | 11 | SEQ ID NO: 728 | SEQ ID NO: 1292 | 0.55416963 |
| Threonine; 3; 7 | 7 | SEQ ID NO: 729 | SEQ ID NO: 1293 | 0.54915125 |
| AsparticAcid; 2; 9 | 9 | SEQ ID NO: 730 | SEQ ID NO: 1294 | 0.54802537 |
| Lysine; 1; 7 | 7 | SEQ ID NO: 731 | SEQ ID NO: 1295 | 0.54460274 |
| GlutamicAcid; 1; 15 | 15 | SEQ ID NO: 732 | SEQ ID NO: 1296 | 0.54206866 |
| AsparticAcid; 2; 15 | 15 | SEQ ID NO: 733 | SEQ ID NO: 1297 | 0.526979 |
| AsparticAcid; 1; 15 | 15 | SEQ ID NO: 734 | SEQ ID NO: 1298 | 0.52579234 |
| Glutamine; 3; 9 | 9 | SEQ ID NO: 735 | SEQ ID NO: 1299 | 0.52493949 |
| GlutamicAcid; 1; 50 | 50 | SEQ ID NO: 736 | SEQ ID NO: 1300 | 0.52368253 |
| GlutamicAcid; 3; 8 | 8 | SEQ ID NO: 737 | SEQ ID NO: 1301 | 0.51955266 |
| GlutamicAcid; 1; 35 | 35 | SEQ ID NO: 738 | SEQ ID NO: 1302 | 0.5179103 |
| Proline; 1; 11 | 11 | SEQ ID NO: 739 | SEQ ID NO: 1303 | 0.51721922 |
| GlutamicAcid; 3; 54 | 54 | SEQ ID NO: 740 | SEQ ID NO: 1304 | 0.51510696 |
| AsparticAcid; 2; 35 | 35 | SEQ ID NO: 741 | SEQ ID NO: 1305 | 0.51046316 |
| Alanine; 3; 6 | 6 | SEQ ID NO: 742 | SEQ ID NO: 1306 | 0.50902755 |
| Glutamine; 1; 7 | 7 | SEQ ID NO: 743 | SEQ ID NO: 1307 | 0.50677669 |
| Alanine; 3; 7 | 7 | SEQ ID NO: 744 | SEQ ID NO: 1308 | 0.50591148 |
| Lysine; 2; 5 | 5 | SEQ ID NO: 745 | SEQ ID NO: 1309 | 0.50454904 |
| Glycine; 3; 7 | 7 | SEQ ID NO: 746 | SEQ ID NO: 1310 | 0.49495873 |
| AsparticAcid; 3; 0 | 0 | SEQ ID NO: 747 | SEQ ID NO: 1311 | 0.48978651 |
| Glutamine; 1; 32 | 32 | SEQ ID NO: 748 | SEQ ID NO: 1312 | 0.48972298 |
| Tyrosine; 2; 69 | 69 | SEQ ID NO: 749 | SEQ ID NO: 1313 | 0.48765802 |
| GlutamicAcid; 2; 7 | 7 | SEQ ID NO: 750 | SEQ ID NO: 1314 | 0.48459406 |
| Histidine; 1; 7 | 7 | SEQ ID NO: 751 | SEQ ID NO: 1315 | 0.48125191 |
| Arginine; 2; 5 | 5 | SEQ ID NO: 752 | SEQ ID NO: 1316 | 0.4801544 |
| Serine; 3; 9 | 9 | SEQ ID NO: 753 | SEQ ID NO: 1317 | 0.47920968 |
| Tryptophan; 1; 53 | 53 | SEQ ID NO: 754 | SEQ ID NO: 1318 | 0.47320791 |
| Serine; 3; 1 | 1 | SEQ ID NO: 755 | SEQ ID NO: 1319 | 0.47295365 |
| Lysine; 2; 34 | 34 | SEQ ID NO: 756 | SEQ ID NO: 1320 | 0.47168537 |
| Lysine; 3; 7 | 7 | SEQ ID NO: 757 | SEQ ID NO: 1321 | 0.47075243 |
| Glycine; 3; 9 | 9 | SEQ ID NO: 758 | SEQ ID NO: 1322 | 0.46950237 |
| AsparticAcid; 3; 9 | 9 | SEQ ID NO: 759 | SEQ ID NO: 1323 | 0.4685333 |
| Threonine; 2; 3 | 3 | SEQ ID NO: 760 | SEQ ID NO: 1324 | 0.46756614 |
| Glycine; 3; 8 | 8 | SEQ ID NO: 761 | SEQ ID NO: 1325 | 0.46569305 |
| GlutamicAcid; 1; 10 | 10 | SEQ ID NO: 762 | SEQ ID NO: 1326 | 0.46430578 |
| AsparticAcid; 3; 4 | 4 | SEQ ID NO: 763 | SEQ ID NO: 1327 | 0.4639914 |
| Serine; 3; 5 | 5 | SEQ ID NO: 764 | SEQ ID NO: 1328 | 0.46366844 |
| Serine; 3; 7 | 7 | SEQ ID NO: 765 | SEQ ID NO: 1329 | 0.45082917 |

TABLE 4
Tiling Repressors

| Gene | Sequence | Avg Day 5 Score |
|---|---|---|
| KRBOX1 | SEQ ID NO: 766 | 7.8278029 |
| ZNF461 | SEQ ID NO: 767 | 7.7007641 |
| ZNF875 | SEQ ID NO: 768 | 7.41648619 |
| ZNF57 | SEQ ID NO: 769 | 7.33652783 |
| CBX5 | SEQ ID NO: 770 | 6.80089192 |
| CBX3 | SEQ ID NO: 771 | 6.7879261 |
| CBX1 | SEQ ID NO: 772 | 6.68230107 |
| CTCF | SEQ ID NO: 773 | 6.25722327 |
| RYBP | SEQ ID NO: 774 | 6.18591064 |
| IRF2BP1 | SEQ ID NO: 775 | 5.6588422 |
| MGA | SEQ ID NO: 776 | 5.6156366 |
| CBX7 | SEQ ID NO: 777 | 5.00371758 |
| IKZF5 | SEQ ID NO: 778 | 4.98433253 |
| REST | SEQ ID NO: 779 | 4.52776703 |
| CBX4 | SEQ ID NO: 780 | 4.52247518 |
| KLF10 | SEQ ID NO: 781 | 4.49441205 |
| SCMH1 | SEQ ID NO: 782 | 4.38546257 |
| SCML2 | SEQ ID NO: 783 | 4.3468225 |
| HIVEP3 | SEQ ID NO: 784 | 4.34368367 |
| HSF2 | SEQ ID NO: 785 | 4.31444003 |
| MBD1 | SEQ ID NO: 786 | 4.25873478 |
| BAZ2A | SEQ ID NO: 787 | 4.17482075 |
| CHD4 | SEQ ID NO: 788 | 4.15410157 |
| ATF7IP | SEQ ID NO: 789 | 4.13988404 |
| PCGF2 | SEQ ID NO: 790 | 4.08140835 |
| HSF1 | SEQ ID NO: 791 | 3.89748786 |
| WIZ | SEQ ID NO: 792 | 3.88045025 |
| CBX7 | SEQ ID NO: 793 | 3.86396312 |
| UHRF1 | SEQ ID NO: 794 | 3.85377779 |
| MGA | SEQ ID NO: 795 | 3.82825762 |
| SIN3B | SEQ ID NO: 796 | 3.67861396 |
| MECP2 | SEQ ID NO: 797 | 3.54786112 |
| ATF7IP | SEQ ID NO: 798 | 3.46711815 |
| INSM2 | SEQ ID NO: 799 | 3.41894392 |
| KDM5B | SEQ ID NO: 800 | 3.41515767 |
| TRIM24 | SEQ ID NO: 801 | 3.34747947 |
| MBD1 | SEQ ID NO: 802 | 3.31964355 |
| SIN3A | SEQ ID NO: 803 | 3.30578618 |
| DMD | SEQ ID NO: 804 | 3.29433336 |
| ZNF827 | SEQ ID NO: 805 | 3.26257067 |
| HIPK2 | SEQ ID NO: 806 | 3.2515196 |
| MBD1 | SEQ ID NO: 807 | 3.18634467 |
| TET2 | SEQ ID NO: 808 | 3.17106197 |
| CBX8 | SEQ ID NO: 809 | 3.14200153 |
| HES3 | SEQ ID NO: 810 | 3.13473999 |
| TRPS1 | SEQ ID NO: 811 | 3.11964253 |
| TRIM24 | SEQ ID NO: 812 | 3.10950731 |
| TET1 | SEQ ID NO: 813 | 3.01568183 |
| BAZ2B | SEQ ID NO: 814 | 2.99449946 |
| ATRX | SEQ ID NO: 815 | 2.99299407 |
| DMD | SEQ ID NO: 816 | 2.95216006 |
| TRIM24 | SEQ ID NO: 817 | 2.94621718 |
| AHRR | SEQ ID NO: 818 | 2.91519283 |
| CBX4 | SEQ ID NO: 819 | 2.8451435 |
| AUTS2 | SEQ ID NO: 820 | 2.80116399 |
| PLAG1 | SEQ ID NO: 821 | 2.78382024 |
| ZNF827 | SEQ ID NO: 822 | 2.76221951 |
| TET3 | SEQ ID NO: 823 | 2.67090027 |
| RCOR3 | SEQ ID NO: 824 | 2.6595258 |
| DNMT3B | SEQ ID NO: 825 | 2.65664419 |
| IKZF2 | SEQ ID NO: 826 | 2.65607702 |
| DNMT3B | SEQ ID NO: 827 | 2.64875126 |
| ZNF827 | SEQ ID NO: 828 | 2.6349517 |
| ATF7IP | SEQ ID NO: 829 | 2.62333283 |
| KDM5C | SEQ ID NO: 830 | 2.62226329 |
| SUV39H1 | SEQ ID NO: 831 | 2.61048387 |
| HES1 | SEQ ID NO: 832 | 2.54719284 |
| HIVEP3 | SEQ ID NO: 833 | 2.54212172 |
| ZNF446 | SEQ ID NO: 834 | 2.53378337 |
| HEY2 | SEQ ID NO: 835 | 2.52262483 |
| PRDM11 | SEQ ID NO: 836 | 2.50622782 |
| PHF19 | SEQ ID NO: 837 | 2.50329194 |
| CBX3 | SEQ ID NO: 838 | 2.46766682 |
| FBRS | SEQ ID NO: 839 | 2.4558407 |
| CIC | SEQ ID NO: 840 | 2.42893202 |
| PCGF6 | SEQ ID NO: 841 | 2.4085226 |
| MNT | SEQ ID NO: 842 | 2.39586222 |
| HSF1 | SEQ ID NO: 843 | 2.39135328 |
| SERTAD2 | SEQ ID NO: 844 | 2.35712893 |
| BMI1 | SEQ ID NO: 845 | 2.34528179 |
| TET3 | SEQ ID NO: 846 | 2.33879669 |
| USP7 | SEQ ID NO: 847 | 2.32632143 |
| TRIM28 | SEQ ID NO: 848 | 2.30375349 |
| L3MBTL3 | SEQ ID NO: 849 | 2.28373726 |
| IKZF4 | SEQ ID NO: 850 | 2.28172595 |
| KDM5B | SEQ ID NO: 851 | 2.26045013 |
| GATAD2A | SEQ ID NO: 852 | 2.24549867 |
| BRMS1 | SEQ ID NO: 853 | 2.23400084 |
| DNMT1 | SEQ ID NO: 854 | 2.18589467 |
| ZNF366 | SEQ ID NO: 855 | 2.17275454 |
| NAB2 | SEQ ID NO: 856 | 2.17138822 |
| KDM1A | SEQ ID NO: 857 | 2.13679618 |
| TGS1 | SEQ ID NO: 858 | 2.11925487 |
| PRDM11 | SEQ ID NO: 859 | 2.07885469 |
| HDAC6 | SEQ ID NO: 860 | 2.03482535 |
| CBX1 | SEQ ID NO: 861 | 2.01737869 |
| TRIM8 | SEQ ID NO: 862 | 1.99485782 |
| MDFI | SEQ ID NO: 863 | 1.98953136 |
| NCOR1 | SEQ ID NO: 864 | 1.98515002 |
| HIVEP3 | SEQ ID NO: 865 | 1.97820999 |
| IKZF1 | SEQ ID NO: 866 | 1.97447058 |
| CDK2AP1 | SEQ ID NO: 867 | 1.96228244 |
| ERF | SEQ ID NO: 868 | 1.94839175 |
| KDM5C | SEQ ID NO: 869 | 1.94183352 |
| ZNF446 | SEQ ID NO: 870 | 1.92348688 |
| CBFA2T3 | SEQ ID NO: 871 | 1.92105376 |
| USP7 | SEQ ID NO: 872 | 1.90807263 |
| SETDB1 | SEQ ID NO: 873 | 1.89374023 |
| PHF1 | SEQ ID NO: 874 | 1.88511381 |
| IKZF3 | SEQ ID NO: 875 | 1.87983916 |
| HDAC4 | SEQ ID NO: 876 | 1.86554151 |
| DNMT3L | SEQ ID NO: 877 | 1.85705086 |
| HDAC9 | SEQ ID NO: 878 | 1.85605728 |
| ZSCAN22 | SEQ ID NO: 879 | 1.83277386 |
| E2F6 | SEQ ID NO: 880 | 1.82276829 |
| KDM5A | SEQ ID NO: 881 | 1.78690918 |
| RBBP7 | SEQ ID NO: 882 | 1.77403489 |
| ATRX | SEQ ID NO: 883 | 1.77379044 |
| ZNF446 | SEQ ID NO: 884 | 1.7514371 |
| GATAD2B | SEQ ID NO: 885 | 1.71132166 |
| TET2 | SEQ ID NO: 886 | 1.67625849 |
| NCOR2 | SEQ ID NO: 887 | 1.65967202 |
| BCOR | SEQ ID NO: 889 | 1.65199942 |
| RBL2 | SEQ ID NO: 890 | 1.65141032 |
| KDM5D | SEQ ID NO: 891 | 1.59348979 |
| TRIM8 | SEQ ID NO: 892 | 1.56673764 |
| KLF3 | SEQ ID NO: 893 | 1.56352551 |
| MTA2 | SEQ ID NO: 894 | 1.53904229 |
| PHF1 | SEQ ID NO: 895 | 1.53480472 |
| RCOR1 | SEQ ID NO: 896 | 1.39637997 |

TABLE 5
Activator Combinations

| | | SEQ |
|---|---|---|
| | | ID |
| Gene | Sequence | NO |
| QNZF | SPFSPVQLHQLRAQILAYKMLARGQ | 1331 |
| | PLPETLQLAVQGGGGGSGEGQSDER | |
| | ALLDQLHTLLSNTDATGLEEIDRAL | |
| | GIPELVNQGQALEPKQGGGGSGFVT | |
| | LKDVGMDFTLGDWEQLGLEQGDTFW | |
| | DTALDNCQDLFLLDPPGGGGSGHEK | |
| | FPSDLDLDMFNGSLECDMESIIRSE | |
| | LMDADGLDFNFDS | |
| NFZ | EGQSDERALLDQLHTLLSNTDATGL | 1332 |
| | EEIDRALGIPELVNQGQALEPKQGG | |
| | GGSGHEKFPSDLDLDMFNGSLECDM | |
| | ESIIRSELMDADGLDFNFDSGGGGS | |
| | GFVTLKDVGMDFTLGDWEQLGLEQG | |
| | DTFWDTALDNCQDLFLLDPP | |
| NZF | EGQSDERALLDQLHTLLSNTDATGL | 1333 |
| | EEIDRALGIPELVNQGQALEPKQGG | |
| | GGSGFVTLKDVGMDFTLGDWEQLGL | |
| | EQGDTFWDTALDNCQDLFLLDPPGG | |
| | GGSGHEKFPSDLDLDMFNGSLECDM | |
| | ESIIRSELMDADGLDFNFDS | |
| NQZF | EGQSDERALLDQLHTLLSNTDATGL | 1334 |
| | EEIDRALGIPELVNQGQALEPKQGG | |
| | GGSGSPFSPVQLHQLRAQILAYKML | |
| | ARGQPLPETLQLAVQGGGGGSGFVT | |
| | LKDVGMDFTLGDWEQLGLEQGDTFW | |
| | DTALDNCQDLFLLDPPGGGGSGHEK | |
| | FPSDLDLDMFNGSLECDMESIIRSE | |
| | LMDADGLDFNFDS | |
| NZQF | EGQSDERALLDQLHTLLSNTDATGL | 1335 |
| | EEIDRALGIPELVNQGQALEPKQGG | |
| | GGSGFVTLKDVGMDFTLGDWEQLGL | |
| | EQGDTFWDTALDNCQDLFLLDPPGG | |
| | GGSGSPFSPVQLHQLRAQILAYKML | |
| | ARGQPLPETLQLAVQGGGGGSGHEK | |
| | FPSDLDLDMFNGSLECDMESIIRSE | |
| | LMDADGLDFNFDS | |
| NZFQ | EGQSDERALLDQLHTLLSNTDATGL | 1336 |
| | EEIDRALGIPELVNQGQALEPKQGG | |
| | GGSGFVTLKDVGMDFTLGDWEQLGL | |
| | EQGDTFWDTALDNCQDLFLLDPPGG | |
| | GGSGHEKFPSDLDLDMFNGSLECDM | |
| | ESIIRSELMDADGLDFNFDSGGGGS | |
| | GSPFSPVQLHQLRAQILAYKMLARG | |
| | QPLPETLQLAVQG | |
| SSXT | AFAAPRQRGKGEITPAAIQKMLDDN | 1337 |
| | NHLIQCIMDSQNKGKTSECSQYQQM | |
| | LHTNLVYLATIADSNQNMQSLLPAP | |
| | PTQNM | |
| F + N + Z | HEKFPSDLDLDMFNGSLECDMESII | 1338 |
| | RSELMDADGLDFNFDSGGGGSGEGQ | |
| | SDERALLDQLHTLLSNTDATGLEEI | |
| | DRALGIPELVNQGQALEPKQGGGGS | |
| | GFVTLKDVGMDFTLGDWEQLGLEQG | |
| | DTFWDTALDNCQDLFLLDPP | |
| F + Z + N | HEKFPSDLDLDMFNGSLECDMESII | 1339 |
| | RSELMDADGLDFNFDSGGGGSGFVT | |
| | LKDVGMDFTLGDWEQLGLEQGDTFW | |
| | DTALDNCQDLFLLDPPGGGGSGEGQ | |
| | SDERALLDQLHTLLSNTDATGLEEI | |
| | DRALGIPELVNQGQALEPKQ | |
| Z + N + F | FVTLKDVGMDFTLGDWEQLGLEQGD | 1340 |
| | TFWDTALDNCQDLFLLDPPGGGGSG | |
| | EGQSDERALLDQLHTLLSNTDATGL | |
| | EEIDRALGIPELVNQGQALEPKQGG | |
| | GGSGHEKFPSDLDLDMFNGSLECDM | |
| | ESIIRSELMDADGLDFNFDS | |
| Z + F + N | FVTLKDVGMDFTLGDWEQLGLEQGD | 1341 |
| | TFWDTALDNCQDLFLLDPPGGGGSG | |
| | HEKFPSDLDLDMFNGSLECDMESII | |
| | RSELMDADGLDFNFDSGGGGSGEGQ | |
| | SDERALLDQLHTLLSNTDATGLEEI | |
| | DRALGIPELVNQGQALEPKQ | |
| N + Z | EGQSDERALLDQLHTLLSNTDATGL | 1342 |
| | EEIDRALGIPELVNQGQALEPKQGG | |
| | GGSGFVTLKDVGMDFTLGDWEQLGL | |
| | EQGDTFWDTALDNCQDLFLLDPP | |
| N + F | EGQSDERALLDQLHTLLSNTDATGL | 1343 |
| | EEIDRALGIPELVNQGQALEPKQGG | |
| | GGSGHEKFPSDLDLDMFNGSLECDM | |
| | ESIIRSELMDADGLDFNFDS | |
| Z + F + N | FVTLKDVGMDFTLGDWEQLGLEQGD | 1344 |
| | TFWDTALDNCQDLFLLDPPGGGGSG | |
| | HEKFPSDLDLDMFNGSLECDMESII | |
| | RSELMDADGLDFNFDS | |

TABLE 6
Repressors

| | | SEQ ID |
|---|---|---|
| Gene | Sequence | NO |
| ZNF705F_ | HSLEKVTFEDVAIDFTQEEWDMMDTSKR | 32 |
| KRAB | KLYRDVMLENISHLVSLGYQISKSYIIL | |
| | QLEQGKELWREGRVFLQDQNPDRE | |
| ZNF471_ | NVEVVKVMPQDLVTFKDVAIDFSQEEWQ | 36 |
| KRAB | WMNPAQKRLYRSMMLENYQSLVSLGLCI | |
| | SKPYVISLLEQGREPWEMTSEMTR | |
| RYBP + | PRLKNVDRSTAQQLAVTVGNVTVIITDF | 1345 |
| MGA1 + 2 | KEKTGGGGSGIKEEPLDDYDGSSSSPEN | |
| | DDLFMMPRIVNVTSLATEGGLVDMGGS | |
| RYBP_ | SKKNSHKKTRPRLKNVDRSSAQHLEVTV | 1346 |
| YAF2_ | GDLTVIITDFKEKTKS | |
| trim | | |
| MGA1 + 2 | IKEEPLDDYDGSSSSPENDDLFMMPRIV | 1347 |
| | NVTSLATEGGLVDMGGS | |

TABLE 7
Effector domains that function as activators or repressors in
at least one target gene context

| | Domain | SEQ ID | |
|---|---|---|---|
| Gene | Type | NO. | Extended Domain sequence |
| ACK1 | SH3_9 | 1348 | ALRDFLLEAQPTDMRALQDFEEPDKLHIQMNDVITVIEGR |
| | | | AENYWWRGQNTRTLCVGPFPRNVVTSVAGLSAQDISQPLQ |
| ALX3 | Homeodomain | 1349 | SMELAKNKSKKRRNRTTFSTFQLEELEKVFQKTHYPDVYA |
| | | | REQLALRTDLTEARVQVWFQNRRAKWRKRERYGKIQEGRN |
| ANR17 | Ank_2 | 1350 | GAQVNMPADSFESPLTLAACGGHVELAALLIERGASLEEV |
| | | | NDEGYTPLMEAAREGHEEMVALLLGQGANINAQTEETQET |
| APBB1 | WW | 586 | GSPSYGSPEDTDSFWNPNAFETDSDLPAGWMRVQDTSGTY |
| | | | YWHIPTGTTQWEPPGRASPSQGSSPQEESQLTWTGFAHGE |
| ASCL4 | HLH | 1351 | LPVPLDSAFEPAFLRKRNERERQRVRCVNEGYARLRDHLP |
| | | | RELADKRLSKVETLRAAIDYIKHLQELLERQAWGLEGAAG |
| ASPM | IQ | 1352 | KRHQEREKAARIIQLAVINFLAKQRLRKRVNAALVIQKYW |
| | | | RRVLAQRKLLMLKKEKLEKVQNKAASLIQGYWRRYSTRQR |
| ASPM | IQ | 1353 | KLTAVRTQAVICIQSYYRGFKVRKDIQNMHRAATLIQSFY |
| | | | RMHRAKVDYETKKTAIVVIQNYYRLYVRVKTERKNFLAVQ |
| ATX3 | UIM | 1354 | IGEELAQLKEQRVHKTDLERVLEANDGSGMLDEDEEDLQR |
| | | | ALALSRQEIDMEDEEADLRRAIQLSMQGSSRNISQDMTQT |
| BRD3 | BET | 1355 | EEEEEGLPMSYDEKRQLSLDINRLPGEKLGRVVHIIQSRE |
| | | | PSLRDSNPDEIEIDFETLKPTTLRELERYVKSCLQKKQRK |
| BRD4 | BET | 1356 | EEEDKCKPMSYEEKRQLSLDINKLPGEKLGRVVHIIQSRE |
| | | | PSLKNSNPDEIEIDFETLKPSTLRELERYVTSCLRKKRKP |
| CACO1 | Zn-C2H2_12 | 598 | SGGEEANLLLPELGSAFYDMASGFTVGTLSETSTGGPATP |
| | | | TWKECPICKERFPAESDKDALEDHMDGHFFFSTQDPFTFE |
| CBX1 | Chromo | 1357 | NKKKVEEVLEEEEEEYVVEKVLDRRVVKGKVEYLLKWKGF |
| | | | SDEDNTWEPEENLDCPDLIAEFLQSQKTAHETDKSEGGKR |
| CBX1 | Chromo_shadow | 168 | EESEKPRGFARGLEPERIIGATDSSGELMFLMKWKNSDEA |
| | | | DLVPAKEANVKCPQVVISFYEERLTWHSYPSEDDDKKDDK |
| CBX3 | Chromo_shadow | 438 | SKKKRDAADKPRGFARGLDPERIIGATDSSGELMFLMKWK |
| | | | DSDEADLVLAKEANMKCPQIVIAFYEERLTWHSCPEDEAQ |
| CBX5 | Chromo_shadow | 380 | QSNDIARGFERGLEPEKIIGATDSCGDLMFLMKWKDTDEA |
| | | | DLVLAKEANVKCPQIVIAFYEERLTWHAYPEDAENKEKET |
| CBX7 | Chromo | 540 | ELSAIGEQVFAVESIRKKRVRKGKVEYLVKWKGWPPKYST |
| | | | WEPEEHILDPRLVMAYEEKEERDRASGYRKRGPKPKRLLL |
| CDY2 | Chromo | 1358 | ASQEFEVEAIVDKRQDKNGNTQYLVRWKGYDKQDDTWEPE |
| | | | QHLMNCEKCVHDFNRRQTEKQKKLTWTTTSRIFSNNARRR |
| CDYL2 | Chromo | 510 | ASGDLYEVERIVDKRKNKKGKWEYLIRWKGYGSTEDTWEP |
| | | | EHHLLHCEEFIDEFNGLHMSKDKRIKSGKQSSTSKLLRDS |
| CEBPD | bZIP_2 | 1359 | AREKSAGKRGPDRGSPEYRQRRERNNIAVRKSRDKAKRRN |
| | | | QEMQQKLVELSAENEKLHQRVEQLTRDLAGLRQFFKQLPS |
| CMTR1 | G-patch | 1360 | AFKADSLVEGTSSRYSMYNSVSQKLMAKMGFREGEGLGKY |
| | | | SQGRKDIVEASSQKGRRGLGLTLRGFDQELNVDWRDEPEP |
| CRTC2 | TORC_C | 594 | GPNIILTGDSSPGFSKEIAAALAGVPGFEVSAAGLELGLG |
| | | | LEDELRMEPLGLEGLNMLSDPCALLPDPAVEESFRSDRLQ |
| CYBP | Siah- | 1361 | ASEELQKDLEEVKVLLEKATRKRVRDALTAEKSKIETEIK |
| | Interact_N | | NKMQQKSQKKAELLDNEKPAAVVAPITTGYTVKISNYGWD |
| DPY30 | Dpy-30 | 616 | EYGLTDNVERIVENEKINAEKSSKQKVDLQSLPTRAYLDQ |
| | | | TVVPILLQGLAVLAKERPPNPIEFLASYLLKNKAQFEDRN |
| EPAS1 | HIF-1a_CTAD | 1362 | PGENSKSRFPPQCYATQYQDYSLSSAHKVSGMASRLLGPS |
| | | | FESYLLPELTRYDCEVNVPVLGSSTLLQGGDLLRALDQAT |
| ESX1 | Homeodomain | 1363 | TAEGPQPPERKRRRRTAFTQFQLQELENFFDESQYPDVVA |
| | | | RERLAARLNLTEDRVQVWFQNRRAKWKRNQRVLMLRNTAT |
| FOS | bZIP_1 | 1364 | GKVEQLSPEEEEKRRIRRERNKMAAAKCRNRRRELTDTLQ |
| | | | AETDQLEDEKSALQTEIANLLKEKEKLEFILAAHRPACKI |
| FOX01 | FOXO-TAD | 568 | GGYSSVSSCNGYGRMGLLHQEKLPSDLDGMFIERLDCDME |
| | | | SIIRNDLMDGDTLDFNFDNVLPNQSFPHSVKTTTHSWVSG |
| FOXO3 | FOXO-TAD | 566 | DSLSGSSLYSTSANLPVMGHEKFPSDLDLDMFNGSLECDM |
| | | | ESIIRSELMDADGLDFNFDSLISTQNVVGLNVGNFTGAKQ |
| HAND2 | HLH | 1365 | AGPPGLGGPRPVKRRGTANRKERRRTQSINSAFAELRECI |
| | | | PNVPADTKLSKIKTLRLATSYIAYLMDLLAKDDQNGEAEA |
| HERC2 | Cyt-b5 | 514 | TLIRKADLENHNKDGGFWTVIDGKVYDIKDFQTQSLTGNS |
| | | | ILAQFAGEDPVVALEAALQFEDTRESMHAFCVGQYLEPDQ |
| HNRPM | RRM_1 | 1366 | RKACQIFVRNLPFDFTWKMLKDKFNECGHVLYADIKMENG |
| | | | KSKGCGVVKFESPEVAERACRMMNGMKLSGREIDVRIDRN |
| HXB13 | Homeodomain | 1367 | QHPPDACAFRRGRKKRIPYSKGQLRELEREYAANKFITKD |
| | | | KRRKISAATSLSERQITIWFQNRRVKEKKVLAKVKNSATP |
| HXC10 | Homeodomain | 1368 | TTGNWLTAKSGRKKRCPYTKHQTLELEKEFLFNMYLTRER |
| | | | RLEISKTINLTDRQVKIWFQNRRMKLKKMNRENRIRELTS |
| ID1 | HLH | 542 | GGAGARLPALLDEQQVNVLLYDMNGCYSRLKELVPTLPQN |
| | | | RKVSKVEILQHVIDYIRDLQLELNSESEVGTPGGRGLPVR |
| ID2 | HLH | 524 | SDHSLGISRSKTPVDDPMSLLYNMNDCYSKLKELVPSIPQ |
| | | | NKKVSKMEILQHVIDYILDLQIALDSHPTIVSLHHQRPGQ |
| ID3 | HLH | 1369 | SLAIARGRGKGPAAEEPLSLLDDMINHCYSRLRELVPGVP |
| | | | RGTQLSQVEILQRVIDYILDLQVVLAEPAPGPPDGPHLPI |
| | | | Q |
| IKKA | IKKbetaNEMObind | 602 | LVGSSLEGAVTPQTSAWLPPTSAEHDHSLSCVVTPQDGET |
| | | | SAQMIEENLNCLGHLSTIIHEANEEQGNSMMNLDWSWLTE |
| IQGA1 | IQ | 1370 | GLITRLQARCRGYLVRQEFRSRMNFLKKQIPAITCIQSQW |
| | | | RGYKQKKAYQDRLAYLRSHKDEVVKIQSLARMHQARKRYR |
| ITCH | WW | 624 | SGLIIPLTISGGSGPRPLNPVTQAPLPPGWEQRVDQHGRV |
| | | | YYVDHVEKRTTWDRPEPLPPGWERRVDNMGRIYYVDHFTR |
| ITCH | WW | 1371 | NQRFIYGNQDLFATSQSKEFDPLGPLPPGWEKRTDSNGRV |
| | | | YFVNHNTRITQWEDPRSQGQLNEKPLPEGWEMRFTVDGIP |
| JDP2 | bZIP_1 | 1372 | QPVKSELDEEEERRKRRREKNKVAAARCRNKKKERTEFLQ |
| | | | RESERLELMNAELKTQIEELKQERQQLILMLNRHRPTCIV |
| KIBRA | WW | 578 | PRPELPLPEGWEEARDFDGKVYYIDHTNRTTSWIDPRDRY |
| | | | TKPLTFADCISDELPLGWEEAYDPQVGDYFIDHNTKTTQI |
| MAGI3 | WW | 1373 | VPSYNQTNSSMDFRNYMMRDETLEPLPKNWEMAYTDTGMI |
| | | | YFIDHNTKTTTWLDPRLCKKAKAPEDCEDGELPYGWEKIE |
| MBD2 | MBDa | 1374 | LQKNKQRLRNDPLNQNKGKPDLNTTLPIRQTASIFKQPVT |
| | | | KVTNHPSNKVKSDPQRMNEQPRQLFWEKRLQGLSASDVTE |
| MPP8 | Chromo | 326 | AEAFGDSEEDGEDVFEVEKILDMKTEGGKVLYKVRWKGYT |
| | | | SDDDTWEPEIHLEDCKEVLLEFRKKIAENKAKAVRKDIQR |
| MYBA | LMSTEN | 570 | FYIPVQIPGYQYVSPEGNCIEHVQPTSAFIQQPFIDEDPD |
| | | | KEKKIKELEMLLMSAENEVRRKRIPSQPGSFSSWSGSFLM |
| MYB | LMSTEN | 572 | EAQNVSSHVPYPVALHVNIVNVPQPAAAAIQRHYNDEDPE |
| | | | KEKRIKELELLLMSTENELKGQQVLPTQNHTCSYPGWHST |
| NCOA3 | Nuc_rec_ | 580 | LRNSLDDLVGPPSNLEGQSDERALLDQLHTLLSNTDATGL |
| | co-act | | EEIDRALGIPELVNQGQALEPKQDAFQGQEAAVMMDQKAG |
| NKX61 | Homeodomain | 1375 | GSILLDKDGKRKHTRPTFSGQQIFALEKTFEQTKYLAGPE |
| | | | RARLAYSLGMTESQVKVWFQNRRTKWRKKHAAEMATAKKK |
| P53 | P53_tetramer | 1376 | LPPGSTKRALPNNTSSSPQPKKKPLDGEYFTLQIRGRERF |
| | | | EMFRELNEALELKDAQAGKEPGGSRAHSSHLKSKKGQSTS |
| P53 | TAD2 | 1377 | PSVEPPLSQETFSDLWKLLPENNVLSPLPSQAMDDLMLSP |
| | | | DDIEQWFTEDPGPDEAPRMPEAAPPVAPAPAAPTPAAPAP |
| P66A | P66_CC | 1378 | NGLTTVALKETSTEALMKSSPEERERMIKQLKEELRLEEA |
| | | | KLVLLKKLRQSQIQKEATAQKPTGSVGSTVTTPPPLVRGT |
| PHF19 | PHD | 1379 | KDIQHAGVPGEEPKCNICLGKTSGPLNEILICGKCGLGYH |
| | | | QQCHIPIAGSADQPLLTPWFCRRCIFALAVRKGGALKKGA |
| PTK6 | SH3_1 | 1380 | VSRDQAHLGPKYVGLWDFKSRTDEELSFRAGDVFHVARKE |
| | | | EQWWWATLLDEAGGAVAQGYVPHNYLAERETVESEPWFFG |
| PYGO1 | PHD | 600 | RHGHSSSDPVYPCGICTNEVNDDQDAILCEASCQKWFHRI |
| | | | CTGMTETAYGLLTAEASAVWGCDTCMADKDVQLMRTRETF |
| RBAK | KRAB | 142 | NTLQGPVSFKDVAVDFTQEEWQQLDPDEKITYRDVMLENY |
| | | | SHLVSVGYDTTKPNVIIKLEQGEEPWIMGGEFPCQHSPEA |
| RYBP | YAF2_RYBP | 364 | PSEANSIQSANATTKTSETNHTSRPRLKNVDRSTAQQLAV |
| | | | TVGNVTVIITDFKEKTRSSSTSSSTVTSSAGSEQQNQSSS |
| SAV1 | WW | 634 | HASGIGRVAATSLGNLTNHGSEDLPLPPGWSVDWTMRGRK |
| | | | YYIDHNTNTTHWSHPLEREGLPPGWERVESSEFGTYYVDH |
| SCX | HLH | 548 | GGGPGGRPGREPRQRHTANARERDRTNSVNTAFTALRTLI |
| | | | PTEPADRKLSKIETLRLASSYISHLGNVLLAGEACGDGQP |
| SF01 | zf-CCHC | 1381 | ELARLNGTLREDDNRILRPWQSSETRSITNTTVCTKCGGA |
| | | | GHIASDCKFQRPGDPQSAQDKARMDKEYLSLMAELGEAPV |
| SF3A3 | Telomere | 1382 | SSALTHAGAHLDLSAFSSWEELASLGLDRLKSALLALGLK |
| | _Sde2_2 | | CGGTLEERAQRLFSTKGKSLESLDTSLFAKNPKSKGTKRD |
| SGT1 | SGS | 1383 | NWDKLVGEIKEEEKNEKLEGDAALNRLFQQIYSDGSDEVK |
| | | | RAMNKSFMESGGTVLSTNWSDVGKRKVEINPPDDMEWKKY |
| SGTA | SGTA_dimer | 1384 | DNKKRLAYANIQFLHDQLRHGGLSSDAQESLEVAIQCLET |
| | | | AFGVTVEDSDLALPQTLPEIFEAAATGKEMPQDLRSPART |
| SMRC2 | Myb_DNA- | 644 | MYTKKNVPSKSKAAASATREWTEQETLLLLEALEMYKDDW |
| | binding | | NKVSEHVGSRTQDECILHFLRLPIEDPYLEDSEASLGPLA |
| SMRC2 | SWIRM- | 1385 | AFLASVVDPRVASAAAKSALEEFSKMKEEVPTALVEAHVR |
| | assoc_3 | | KVEEAAKVTGKADPAFGLESSGIAGTTSDEPERIEESGND |
| SOX14 | HMG_box | 1386 | KPSDHIKRPMNAFMVWSRGQRRKMAQENPKMHNSEISKRL |
| | | | GAEWKLLSEAEKRPYIDEAKRLRAQHMKEHPDYKYRPRRK |
| SRBP1 | HLH | 1387 | AGSKAPASAQSRGEKRTAHNAIEKRYRSSINDKIIELKDL |
| | | | VVGTEAKLNKSAVLRKAIDYIRFLQHSNQKLKQENLSLRT |
| STAT2 | STAT2_C | 628 | SQTVPEPDQGPVSQPVPEPDLPCDLRHLNTEPMEIFRNCV |
| | | | KIEEIMPNGDPLLAGQNTVDEVYVSRPSHFYTDGPLMPSD |
| STIP1 | TPR_8 | 1388 | KKKDFDTALKHYDKAKELDPTNMTYITNQAAVYFEKGDYN |
| | | | KCRELCEKAIEVGRENREDYRQIAKAYARIGNSYFKEEKY |
| TCRG1 | FF | 1389 | AEIKAARERAIVPLEARMKQFKDMLLERGVSAFSTWEKEL |
| | | | HKIVFDPRYLLLNPKERKQVFDQYVKTRAEEERREKKNKI |
| TCRG1 | FF | 1390 | TKEIDREREQHKREEAIQNFKALLSDMVRSSDVSWSDTRR |
| | | | TLRKDHRWESGSLLEREEKEKLFNEHIEALTKKKREHFRQ |
| TOX | HMG_box | 526 | KDPNEPQKPVSAYALFFRDTQAAIKGQNPNATFGEVSKIV |
| | | | ASMWDGLGEEQKQVYKKKTEAAKKEYLKQLAAYRASLVSK |
| TRI27 | zf-B_box | 1391 | ANVTQLVKQLRTERPSGPGGEMGVCEKHREPLKLYCEEDQ |
| | | | MPICVVCDRSREHRGHSVLPLEEAVEGFKEQIQNQLDHLK |
| UBF1 | HMG_box_2 | 1392 | LKDKFDGRPTKPPPNSYSLYCAELMANMKDVPSTERMVLC |
| | | | SQQWKLLSQKEKDAYHKKCDQKKKDYEVELLRFLESLPEE |
| WBP4 | WW | 640 | YYDLISGASQWEKPEGFQGDLKKTAVKTVWVEGLSEDGFT |
| | | | YYYNTETGESRWEKPDDFIPHTSDLPSSKVNENSLGTLDE |
| WWP1 | WW | 1393 | AAKSRQPDGCMDPVRQQSGNANTETLPSGWEQRKDPHGRT |
| | | | YYVDHNTRTTTWERPQPLPPGWERRVDDRRRVYYVDHNTR |
| WWP1 | WW | 614 | AMQQFNQRYLYSASMLAAENDPYGPLPPGWEKRVDSTDRV |
| | | | YFVNHNTKTTQWEDPRTQGLQNEEPLPEGWEIRYTREGVR |
| WWP2 | WW | 1394 | TPAEGEEPSTSGTQQLPAAAQAPDALPAGWEQRELPNGRV |
| | | | YYVDHNTKTTTWERPLPPGWEKRTDPRGRFYYVDHNTRTT |
| WWP2 | WW | 606 | AMQHFSQRFLYQSSSASTDHDPLGPLPPGWEKRQDNGRVY |
| | | | YVNHNTRTTQWEDPRTQGMIQEPALPPGWEMKYTSEGVRY |
| WWTR1 | WW | 648 | GAAGSPAQQHAHLRQQSYDVTDELPLPPGWEMTFTATGQR |
| | | | YFLNHIEKITTWQDPRKAMNQPLNHMNLHPAVSSTPVPQR |
| YAF2 | YAF2_RYBP | 422 | KDKVEKEKSEKETTSKKNSHKKTRPRLKNVDRSSAQHLEV |
| | | | TVGDLTVIITDFKEKTKSPPASSAASADQHSQSGSSSDNT |
| Z585A | KRAB | 100 | SPQKSSALAPEDHGSSYEGSVSFRDVAIDFSREEWRHLDP |
| | | | SQRNLYRDVMLETYSHLLSVGYQVPEAEVVMLEQGKEPWA |
| Z585B | KRAB | 260 | SPQKSSALAPEDHGSSYEGSVSFRDVAIDFSREEWRHLDL |
| | | | SQRNLYRDVMLETYSHLLSVGYQVPKPEVVMLEQGKEPWA |
| ZF69B | KRAB | 410 | GESLESRVTLGSLTAESQELLTFKDVSVDFTQEEWGQLAP |
| | | | AHRNLYREVMLENYGNLVSVGCQLSKPGVISQLEKGEEPW |
| ZFP1 | KRAB | 272 | NKSQGSVSFTDVTVDFTQEEWEQLDPSQRILYMDVMLENY |
| | | | SNLLSVEVWKADDQMERDHRNPDEQARQFLILKNQTPIEE |
| ZFP69 | KRAB | 238 | RESLEDEVTPGLPTAESQELLTFKDISIDFTQEEWGQLAP |
| | | | AHQNLYREVMLENYSNLVSVGYQLSKPSVISQLEKGEEPW |
| ZHANG | bZIP_1 | 1395 | GGGSGNDNNQAATKSPRKAAAAAARLNRLKKKEYVMGLES |
| | | | RVRGLAAENQELRAENRELGKRVQALQEESRYLRAVLANE |
| ZIC5 | zf_ZIC | 1396 | CKWIDPDELAGLPPPPPPPPPPPPPPPPGAKPCSKTFGTM |
| | | | HELVNHVTVEHVGGPEQSSHVCFWEDCPREGKPFKAKYK |
| ZN114 | KRAB | 1397 | SQDSVTFADVAVNFTKEEWTLLDPAQRNLYRDVMLENSRN |
| | | | LAFIDWATPCKTKDATPQPDILPKRTFPEANRVCLTSISS |
| ZN124 | KRAB | 120 | SGHPGSWEMINSVAFEDVAVNFTQEEWALLDPSQKNLYRD |
| | | | VMQETFRNLASIGNKGEDQSIEDQYKNSSRNLRHIISHSG |
| | | | N |
| ZN157 | KRAB | 184 | SPQRFPALIPGEPGRSFEGSVSFEDVAVDFTRQEWHRLDP |
| | | | AQRTMHKDVMLETYSNLASVGLCVAKPEMIFKLERGEELW |
| ZN229 | KRAB | 348 | HSQASAISQDREEKIMSQEPLSFKDVAVVFTEEELELLDS |
| | | | TQRQLYQDVMQENFRNLLSVGERNPLGDKNGKDTEYIQDE |
| ZN250 | KRAB | 442 | AAARLLPVPAGPQPLSFQAKLTFEDVAVLLSQDEWDRLCP |
| | | | AQRGLYRNVMMETYGNVVSLGLPGSKPDIISQLERGEDPW |
| ZN256 | KRAB | 500 | AAAELTAPAQGIVTFEDVAVYFSWKEWGLLDEAQKCLYHD |
| | | | VMLENLTLTTSLGGSGAGDEEAPYQQSTSPQRVSQVRIPK |
| ZN274 | KRAB | 528 | QEEKQEDAAICPVTVLPEEPVTFQDVAVDFSREEWGLLGP |
| | | | TQRTEYRDVMLETFGHLVSVGWETTLENKELAPNSDIPEE |
| ZN283 | KRAB | 84 | EESHGALISSCNSRTMTDGLVTFRDVAIDFSQEEWECLDP |
| | | | AQRDLYVDVMLENYSNLVSLDLESKTYETKKIFSENDIFE |
| ZN283 | zf-C2H2_6 | 1398 | KPFECKECGKAFSWGSSLVKHERVHTGEKSHECKECGKTF |
| | | | CSGYQLTRHQVFHTGEKPYECKECGKAFNCGSSLVQHERI |
| ZN300 | KRAB | 206 | MKSQGLVSFKDVAVDFTQEEWQQLDPSQRTLYRDVMLENY |
| | | | SHLVSMGYPVSKPDVISKLEQGEEPWIIKGDISNWIYPDE |
| ZN311 | KRAB | 1399 | GSQGNLPQADITLMSQAQESVTFEDVAVNFTNREWQCLTY |
| | | | AQRHLYKDVMLENYGNMVSLGFPFPKPPLISHLEREVDPC |
| ZN317 | KRAB | 124 | DLFVCSGLEPHTPSVGSQESVTFQDVAVDFTEKEWPLLDS |
| | | | SQRKLYKDVMLENYSNLTSLGYQVGKPSLISHLEQEEEPR |
| ZN333 | KRAB | 396 | DKVEEEAMAPGLPTACSQEPVTFADVAVVFTPEEWVFLDS |
| | | | TQRSLYRDVMLENYRNLASVADQLCKPNALSYLEERGEQW |
| ZN33A | KRAB | 68 | NKVEQKSQESVSFKDVTVGFTQEEWQHLDPSQRALYRDVM |
| | | | LENYSNLVSVGYCVHKPEVIFRLQQGEEPWKQEEEFPSQS |
| ZN350 | KRAB | 154 | IQAQESITLEDVAVDFTWEEWQLLGAAQKDLYRDVMLENY |
| | | | SNLVAVGYQASKPDALFKLEQGEQLWTIEDGIHSGACSDI |
| ZN37A | KRAB | 8 | ITSQGSVSFRDVTVGFTQEEWQHLDPAQRTLYRDVMLENY |
| | | | SHLVSVGYCIPKPEVILKLEKGEEPWILEEKFPSQSHLEL |
| ZN420 | KRAB | 458 | ARKLVMFRDVAIDFSQEEWECLDSAQRDLYRDVMLENYSN |
| | | | LVSLDLPSRCASKDLSPEKNTYETELSQWEMSDRLENCDL |
| ZN442 | KRAB | 356 | RSDLFLPDSQTNEERKQYDSVAFEDVAVNFTQEEWALLGP |
| | | | SQKSLYRDVMWETIRNLDCIGMKWEDTNIEDQHRNPRRSL |
| ZN443 | KRAB | 170 | ASVALEDVAVNFTREEWALLGPCQKNLYKDVMQETIRNLD |
| | | | CVVMKWKDQNIEDQYRYPRKNLRCRMLERFVESKDGTQCG |
| ZN460 | KRAB | 468 | AAAWMAPAQESVTFEDVAVTFTQEEWGQLDVTQRALYVEV |
| | | | MLETCGLLVALGDSTKPETVEPIPSHLALPEEVSLQEQLA |
| ZN470 | KRAB | 110 | SQEEVEVAGIKLCKAMSLGSVTFTDVAIDFSQDEWEWLNL |
| | | | AQRSLYKKVMLENYRNLVSVGLCISKPDVISLLEQEKDPW |
| ZN471 | KRAB | 36 | NVEVVKVMPQDLVTFKDVAIDFSQEEWQWMNPAQKRLYRS |
| | | | MMLENYQSLVSLGLCISKPYVISLLEQGREPWEMTSEMTR |
| ZN473 | KRAB | 564 | AEEFVTLKDVGMDFTLGDWEQLGLEQGDTFWDTALDNCQD |
| | | | LFLLDPPRPNLTSHPDGSEDLEPLAGGSPEATSPDVTETK |
| ZN490 | KRAB | 328 | VLQMQNSEHHGQSIKTQTDSISLEDVAVNFTLEEWALLDP |
| | | | GQRNIYRDVMRATFKNLACIGEKWKDQDIEDEHKNQGRNL |
| ZN534 | KRAB | 366 | ALTQGQLSFSDVAIEFSQEEWKCLDPGQKALYRDVMLENY |
| | | | RNLVSLGEDNVRPEACICSGICLPDLSVTSMLEQKRDPWT |
| ZN549 | KRAB | 86 | VITPQIPMVTEEFVKPSQGHVTFEDIAVYFSQEEWGLLDE |
| | | | AQRCLYHDVMLENFSLMASVGCLHGIEAEEAPSEQTLSAQ |
| ZN555 | KRAB | 136 | DSVVFEDVAVDFTLEEWALLDSAQRDLYRDVMLETFQNLA |
| | | | SVDDETQFKASGSVSQQDIYGEKIPKESKIATFTRNVSWA |
| ZN561 | KRAB | 388 | EKTKVERMVEDYLASGYQDSVTFDDVAVDFTPEEWALLDT |
| | | | TEKYLYRDVMLENYMNLASVEWEIQPRTKRSSLQQGFLKN |
| ZN564 | KRAB | 416 | DSVASEDVAVNFTLEEWALLDPSQKKLYRDVMRETFRNLA |
| | | | CVGKKWEDQSIEDWYKNQGRILRNHMEEGLSESKEYDQCG |
| ZN568 | KRAB | 6 | CSQESALSEEEEDTTRPLETVTFKDVAVDLTQEEWEQMKP |
| | | | AQRNLYRDVMLENYSNLVTVGCQVTKPDVIFKLEQEEEPW |
| ZN569 | KRAB | 414 | TESQGTVTFKDVAIDFTQEEWKRLDPAQRKLYRNVMLENY |
| | | | NNLITVGYPFTKPDVIFKLEQEEEPWVMEEEVLRRHWQGE |
| ZN595 | KRAB | 1400 | ELVTFRDVAIEFSPEEWKCLDPAQQNLYRDVMLENYRNLV |
| | | | SLGFVISNPDLVTCLEQIKEPCNLKIHETAAKPPAICSPF |
| ZN597 | KRAB | 584 | ASMPPTPEAQGPILFEDLAVYFSQEECVTLHPAQRSLSKD |
| | | | GTKESLEDAALMGEEGKPEINQQLSLESMELDELALEKYP |
| ZN606 | KRAB | 350 | GSLEEGRRATGLPAAQVQEPVTFKDVAVDFTQEEWGQLDL |
| | | | VQRTLYRDVMLETYGHLLSVGNQIAKPEVISLLEQGEEPW |
| ZN611 | KRAB | 294 | EEAAQKRKGKEPGMALPQGRLTFRDVAIEFSLAEWKCLNP |
| | | | SQRALYREVMLENYRNLEAVDISSKCMMKEVLSTGQGNTE |
| ZN615 | KRAB | 92 | MQAQESLTLEDVAVDFTWEEWQFLSPAQKDLYRDVMLENY |
| | | | SNLVAVGYQASKPDALSKLERGEETCTTEDEIYSRICSEI |
| ZN624 | KRAB | 38 | TQPDEDLHLQAEETQLVKESVTFKDVAIDFTLEEWRLMDP |
| | | | TQRNLHKDVMLENYRNLVSLGLAVSKPDMISHLENGKGPW |
| ZN627 | KRAB | 284 | DSVAFEDVAVNFTLEEWALLDPSQKNLYRDVMRETFRNLA |
| | | | SVGKQWEDQNIEDPFKIPRRNISHIPERLCESKEGGQGEE |
| ZN630 | KRAB | 392 | IESQEPVTFEDVAVDFTQEEWQQLNPAQKTLHRDVMLETY |
| | | | NHLVSVGCSGIKPDVIFKLEHGKDPWIIESELSRWIYPDR |
| ZN649 | KRAB | 108 | TKAQESLTLEDVAVDFTWEEWQFLSPAQKDLYRDVMLENY |
| | | | SNLVSVGYQAGKPDALTKLEQGEPLWTLEDEIHSPAHPEI |
| ZN670 | KRAB | 1401 | DSVSFEDVAVAFTQEEWALLDPSQKNLYRDVMQEIFRNLA |
| | | | SVGNKSEDQNIQDDFKNPGRNLSSHVVERLFEIKEGSQYG |
| ZN674 | KRAB | 282 | AMSQESLTFKDVFVDFTLEEWQQLDSAQKNLYRDVMLENY |
| | | | SHLVSVGHLVGKPDVIFRLGPGDESWMADGGTPVRTCAGE |
| ZN677 | KRAB | 212 | ALSQGLFTFKDVAIEFSQEEWECLDPAQRALYRDVMLENY |
| | | | RNLLSLDEDNIPPEDDISVGFTSKGLSPKENNKEELYHLV |
| ZN709 | KRAB | 402 | DSVVFEDVAVNFTQEEWALLGPSQKKLYRDVMQETFVNLA |
| | | | SIGENWEEKNIEDHKNQGRKLRSHMVERLCERKEGSQFGE |
| ZN718 | KRAB | 204 | ELLTFKDVAIEFSPEEWKCLDTSQQNLYRDVMLENYRNLV |
| | | | SLGVSISNPDLVTSLEQRKEPYNLKIHETAARPPAVCSHF |
| ZN763 | KRAB | 334 | DPVACEDVAVNFTQEEWALLDISQRKLYREVMLETFRNLT |
| | | | SIGKKWKDQNIEYEYQNPRRNFRSLIEGNVNEIKEDSHCG |
| ZN791 | KRAB | 178 | DSVAFEDVSVSFSQEEWALLAPSQKKLYRDVMQETFKNLA |
| | | | SIGEKWEDPNVEDQHKNQGRNLRSHTGERLCEGKEGSQCA |
| ZN805 | KRAB | 330 | AMALTDPAQVSVTFDDVAVTFTQEEWGQLDLAQRTLYQEV |
| | | | MLENCGLLVSLGCPVPRPELIYHLEHGQEPWTRKEDLSQG |
| ZN823 | KRAB | 490 | DSVAFEDVAVNFTQEEWALLGPSQKSLYRNVMQETIRNLD |
| | | | CIEMKWEDQNIGDQCQNAKRNLRSHTCEIKDDSQCGETFG |
| ZN862 | KRAB | 14 | QDPSAEGLSEEVPVVFEELPVVFEDVAVYFTREEWGMLDK |
| | | | RQKELYRDVMRMNYELLASLGPAAAKPDLISKLERRAAPW |
| ZNF12 | KRAB | 242 | NKSLGPVSFKDVAVDFTQEEWQQLDPEQKITYRDVMLENY |
| | | | SNLVSVGYHIIKPDVISKLEQGEEPWIVEGEFLLQSYPDE |
| ZNF20 | KRAB | 286 | MFQDSVAFEDVAVSFTQEEWALLDPSQKNLYRDVMQETFK |
| | | | NLTSVGKTWKVQNIEDEYKNPRRNLSLMREKLCESKESHH |
| ZNF34 | KRAB | 426 | RKPNPQAMAALFLSAPPQAEVTFEDVAVYLSREEWGRLGP |
| | | | AQRGLYRDVMLETYGNLVSLGVGPAGPKPGVISQLERGDE |
| ZNF44 | KRAB | 230 | TLPRGQPEVLEWGLPKDQDSVAFEDVAVNFTHEEWALLGP |
| | | | SQKNLYRDVMRETIRNLNCIGMKWENQNIDDQHQNLRRNP |
| ZNF57 | KRAB | 82 | DSVVFEDVAVDFTLEEWALLDSAQRDLYRDVMLETFRNLA |
| | | | SVDDGTQFKANGSVSLQDMYGQEKSKEQTIPNFTGNNSCA |
| ZNF7 | KRAB | 42 | EVVTFGDVAVHFSREEWQCLDPGQRALYREVMLENHSSVA |
| | | | GLAGFLVFKPELISRLEQGEEPWVLDLQGAEGTEAPRTSK |
| ZNF8 | KRAB | 194 | DEGVAGVMSVGPPAARLQEPVTFRDVAVDFTQEEWGQLDP |
| | | | TQRILYRDVMLETFGHLLSIGPELPKPEVISQLEQGTELW |

1. A synthetic transcription factor comprising one or more transcriptional activator domains, one or more transcriptional repressor domains, or a combination thereof fused to a heterologous DNA binding domain,
wherein at least one of the one or more transcriptional activator domains or at least one of the one or more transcriptional repressor domains comprises an amino acid sequence having at least 70% identity to any of SEQ ID NOs: 1-896, 1346-1401.

2. (canceled)

3. The synthetic transcription factor of claim 1 or 2, wherein the one or more transcriptional activator domains comprise an amino acid sequence having at least 70% identity to any of SEQ ID NOs: 563, 564, 565, 566, 575, 576, 579, and 580.

4. The synthetic transcription factor of claim 3, wherein the one or more transcriptional activator domains comprises an amino acid sequence having: SEQ ID NO: 563 or SEQ ID NO: 564; SEQ ID NO: 565 or SEQ ID NO: 566; SEQ ID NO: 575 or SEQ ID NO: 576; SEQ ID NO: 579 or SEQ ID NO: 580; or a combination thereof.

5. The synthetic transcription factor of claim 4, wherein the one or more transcriptional activator domains comprises an amino acid sequence having two or more of SEQ ID NOs: 563, 565, 575, and 579.

6. The synthetic transcription factor of claim 5, wherein the one or more transcriptional activator domains comprises an amino acid sequence having SEQ ID NOS: 563, 565, and 579 or SEQ ID NO: 575.

7. (canceled)

8. The synthetic transcription factor of claim 1, wherein the synthetic transcription factor comprises an amino acid sequence having at least 70% identity to any of SEQ ID NOs: 1331-1344.

9. (canceled)

10. The synthetic transcription factor of claim 1, wherein the one or more transcriptional repressor domains comprise an amino acid sequence having at least 70% identity to any of SEQ ID NOs: 32, 36, 363, or a combination thereof.

11. The synthetic transcription factor of claim 10, wherein the synthetic transcription factor comprises an amino acid sequence of SEQ ID NO: 1345.

12. The synthetic transcription factor of claim 1, wherein the heterologous DNA binding domain is an inducible DNA binding domain.

13. A nucleic acid encoding at least one synthetic transcription factor of claim 1.

14. The nucleic acid of claim 13, wherein the nucleic acid further comprises a cargo gene.

15. The nucleic acid of claim 14, wherein the cargo gene is greater than approximately 1.1 kb and/or less than or equal to approximately 2.3 kb.

16. (canceled)

17. The nucleic acid of claim 14, wherein the cargo gene comprises an antibody, a cytokine, a lysosomal enzyme, a CRISPR/Cas system, or a combination thereof.

18-19. (canceled)

20. A cell comprising at least one synthetic transcription factor of any of claim 1 or one or more nucleic acids encoding thereof.

21-23. (canceled)

24. A kit comprising at least one synthetic transcription factor of claim 1, or one or more nucleic acids encoding thereof, or a cell, composition, or system comprising thereof.

25. A method of modulating the expression of at least one target gene in a cell, the method comprising introducing into the cell at least one synthetic transcription factor of claim 1 or one or more nucleic acids encoding thereof.

26. The method of claim 25, wherein the at least one target gene is an endogenous gene, an exogenous gene, or a combination thereof.

27. The method of claim 25, wherein the cell is in a subject.

28. The method of claim 27, wherein the method comprises administering the at least one synthetic transcription factor, or one or more nucleic acids encoding thereof.

29. The method of claim 25, wherein the gene expression of at least two genes are modulated.

30-31. (canceled)


SEQUENCE LISTING (1444 sequences)

SEQ ID NO: 1 - protein, 41 aa, synthetic construct
LVTFGDVAVDFSQEEWEWLNPIQRNLYRKVMLENYRNLASL

SEQ ID NO: 2 - protein, 80 aa, synthetic construct
NKKLEAVGTGIEPKAMSQGLVTFGDVAVDFSQEEWEWLNPIQRNLYRKVMLENYRNLASLGLCVSKPDVISSLEQGKEPW

SEQ ID NO: 3 - protein, 41 aa, synthetic construct
PVSFQDLTVNFTQEEWQQLDPAQRLLYRDVMLENYSNLVSV

SEQ ID NO: 4 - protein, 80 aa, synthetic construct
KMKKFQIPVSFQDLTVNFTQEEWQQLDPAQRLLYRDVMLENYSNLVSVGYHVSKPDVIFKLEQGEEPWIVEEFSNQNYPD

SEQ ID NO: 5 - protein, 41 aa, synthetic construct
TVTFKDVAVDLTQEEWEQMKPAQRNLYRDVMLENYSNLVTV

SEQ ID NO: 6 - protein, 80 aa, synthetic construct
CSQESALSEEEEDTTRPLETVTFKDVAVDLTQEEWEQMKPAQRNLYRDVMLENYSNLVTVGCQVTKPDVIFKLEQEEEPW

SEQ ID NO: 7 - protein, 41 aa, synthetic construct
SVSFRDVTVGFTQEEWQHLDPAQRTLYRDVMLENYSHLVSV

SEQ ID NO: 8 - protein, 80 aa, synthetic construct
ITSQGSVSFRDVTVGFTQEEWQHLDPAQRTLYRDVMLENYSHLVSVGYCIPKPEVILKLEKGEEPWILEEKFPSQSHLEL

SEQ ID NO: 9 - protein, 40 aa, synthetic construct
QVTFNDVAIDFTHEEWGWLSSAQRDLYKDVMVQNYENLVS

SEQ ID NO: 10 - protein, 80 aa, synthetic construct
PQVTFNDVAIDFTHEEWGWLSSAQRDLYKDVMVQNYENLVSVAGLSVTKPYVITLLEDGKEPWMMEKKLSKGMIPDWESR

SEQ ID NO: 11 - protein, 41 aa, synthetic construct
SVSFKDVTIEFTQEEWQQMAPVQKNLYRDVMLENYSNLVSV

SEQ ID NO: 12 - protein, 80 aa, synthetic construct
PLRFSTLFQEQQKMNISQASVSFKDVTIEFTQEEWQQMAPVQKNLYRDVMLENYSNLVSVGYCCFKPEVIFKLEQGEEPW

SEQ ID NO: 13 - protein, 41 aa, synthetic construct
PVVFEDVAVYFTREEWGMLDKRQKELYRDVMRMNYELLASL

SEQ ID NO: 14 - protein, 80 aa, synthetic construct
QDPSAEGLSEEVPVVFEELPVVFEDVAVYFTREEWGMLDKRQKELYRDVMRMNYELLASLGPAAAKPDLISKLERRAAPW

SEQ ID NO: 15 - protein, 41 aa, synthetic construct
SVTFRDVAIDFSQEEWKWLQPAQRDLYRCVMLENYGHLVSL

SEQ ID NO: 16 - protein, 80 aa, synthetic construct
SQGSVTFRDVAIDFSQEEWKWLQPAQRDLYRCVMLENYGHLVSLGLSISKPDVVSLLEQGKEPWLGKREVKRDLFSVSES

SEQ ID NO: 17 - protein, 41 aa, synthetic construct
SLTFRDVAIEFSLEEWQCLDTAQQNLYRNVMLENYRNLVFL

SEQ ID NO: 18 - protein, 80 aa, synthetic construct
GSLTFRDVAIEFSLEEWQCLDTAQQNLYRNVMLENYRNLVFLGIAAFKPDLIIFLEEGKESWNMKRHEMVEESPVICSHF

SEQ ID NO: 19 - protein, 41 aa, synthetic construct
QVSFKDVCVDFTQEEWYLLDPAQKILYRDVILENYSNLVSV

SEQ ID NO: 20 - protein, 80 aa, synthetic construct
NKSQEQVSFKDVCVDFTQEEWYLLDPAQKILYRDVILENYSNLVSVGYCITKPEVIFKIEQGEEPWILEKGFPSQCHPER

SEQ ID NO: 21 - protein, 41 aa, synthetic construct
LVTFRDVAIDFSQEEWECLDPAQRDLYRDVMLENYSNLISL

SEQ ID NO: 22 - protein, 80 aa, synthetic construct
PHLLVTFRDVAIDFSQEEWECLDPAQRDLYRDVMLENYSNLISLDLESSCVTKKLSPEKEIYEMESLQWENMGKRINHHL

SEQ ID NO: 23 - protein, 41 aa, synthetic construct
SVVFEDVAVDFTQEEWALLDLAQRNLYRDVMLENFQNLASL

SEQ ID NO: 24 - protein, 80 aa, synthetic construct
EEERKTAELQKNRIQDSVVFEDVAVDFTQEEWALLDLAQRNLYRDVMLENFQNLASLGYPLHTPHLISQWEQEEDLQTVK

SEQ ID NO: 25 - protein, 41 aa, synthetic construct
LLTFRDVAIEFSLEEWQCLDTAQKNLYRNVMLENYRNLAFL

SEQ ID NO: 26 - protein, 80 aa, synthetic construct
GLLTFRDVAIEFSLEEWQCLDTAQKNLYRNVMLENYRNLAFLGIAVSKPDLIICLEKEKEPWNMKRDEMVDEPPGICPHF

SEQ ID NO: 27 - protein, 41 aa, synthetic construct
CVTFEDIAIYFSQDEWGLLDEAQRLLYLEVMLENFALVASL

SEQ ID NO: 28 - protein, 80 aa, synthetic construct
RAPTQVTVSPETHMDLTKGCVTFEDIAIYFSQDEWGLLDEAQRLLYLEVMLENFALVASLGCGHGTEDEETPSDQNVSVG

SEQ ID NO: 29 - protein, 41 aa, synthetic construct
SVTFEDVAVVFTDEEWSRLVPIQRDLYKEVMLENYNSIVSL

SEQ ID NO: 30 - protein, 80 aa, synthetic construct
AAVSPTTRCQESVTFEDVAVVFTDEEWSRLVPIQRDLYKEVMLENYNSIVSLGLPVPQPDVIFQLKRGDKPWMVDLHGSE

SEQ ID NO: 31 - protein, 41 aa, synthetic construct
KVTFEDVAIDFTQEEWDMMDTSKRKLYRDVMLENISHLVSL

SEQ ID NO: 32 - protein, 80 aa, synthetic construct
HSLEKVTFEDVAIDFTQEEWDMMDTSKRKLYRDVMLENISHLVSLGYQISKSYIILQLEQGKELWREGRVFLQDQNPDRE

SEQ ID NO: 33 - protein, 41 aa, synthetic construct
SVSFEDVAVNFTLEEWALLDSSQKKLYEDVMQETFKNLVCL

SEQ ID NO: 34 - protein, 80 aa, synthetic construct
DSVSFEDVAVNFTLEEWALLDSSQKKLYEDVMQETFKNLVCLGKKWEDQDIEDDHRNQGKNRRCHMVERLCESRRGSKCG

SEQ ID NO: 35 - protein, 41 aa, synthetic construct
LVTFKDVAIDFSQEEWQWMNPAQKRLYRSMMLENYQSLVSL

SEQ ID NO: 36 - protein, 80 aa, synthetic construct
NVEVVKVMPQDLVTFKDVAIDFSQEEWQWMNPAQKRLYRSMMLENYQSLVSLGLCISKPYVISLLEQGREPWEMTSEMTR

SEQ ID NO: 37 - protein, 41 aa, synthetic construct
SVTFKDVAIDFTLEEWRLMDPTQRNLHKDVMLENYRNLVSL

SEQ ID NO: 38 - protein, 80 aa, synthetic construct
TQPDEDLHLQAEETQLVKESVTFKDVAIDFTLEEWRLMDPTQRNLHKDVMLENYRNLVSLGLAVSKPDMISHLENGKGPW

SEQ ID NO: 39 - protein, 41 aa, synthetic construct
SFSFDDLSVDFTQKEWQLLDPSQKNLYKDVMLENYSSLVSL

SEQ ID NO: 40 - protein, 80 aa, synthetic construct
TMLQESFSFDDLSVDFTQKEWQLLDPSQKNLYKDVMLENYSSLVSLGYEVMKPDVIFKLEQGEEPWVGDGEIPSSDSPEV

SEQ ID NO: 41 - protein, 40 aa, synthetic construct
VVTFGDVAVHFSREEWQCLDPGQRALYREVMLENHSSVAG

SEQ ID NO: 42 - protein, 80 aa, synthetic construct
EVVTFGDVAVHFSREEWQCLDPGQRALYREVMLENHSSVAGLAGFLVFKPELISRLEQGEEPWVLDLQGAEGTEAPRTSK

SEQ ID NO: 43 - protein, 41 aa, synthetic construct
PMTFKDVAVEFTQEEWMMLDSAQRSLYRDVMLENYRNLTSV

SEQ ID NO: 44 - protein, 80 aa, synthetic construct
RNAEEERMIAVFLTTWLQEPMTFKDVAVEFTQEEWMMLDSAQRSLYRDVMLENYRNLTSVEYQLYRLTVISPLDQEEIRN

SEQ ID NO: 45 - protein, 41 aa, synthetic construct
FLAFGDVTVDFTQKEWRLLSPAQRALYREVTLENYSHLVSL

SEQ ID NO: 46 - protein, 80 aa, synthetic construct
GPQGARRQAFLAFGDVTVDFTQKEWRLLSPAQRALYREVTLENYSHLVSLGILHSKPELIRRLEQGEVPWGEERRRRPGP

SEQ ID NO: 47 - protein, 41 aa, synthetic construct
KLTFEDVAIDFTQEEWAMMDTSKRKLYRDVMLENISHLVSL

SEQ ID NO: 48 - protein, 80 aa, synthetic construct
HSLKKLTFEDVAIDFTQEEWAMMDTSKRKLYRDVMLENISHLVSLGYQISKSYIILQLEQGKELWREGRVFLQDQNPNRE

SEQ ID NO: 49 - protein, 41 aa, synthetic construct
LVTLRDVVINFSQEEWEYLDSAQRNLYWDVMMENYSNLLSL

SEQ ID NO: 50 - protein, 80 aa, synthetic construct
MPEVEFPDQFFTVLTMDHELVTLRDVVINFSQEEWEYLDSAQRNLYWDVMMENYSNLLSLDLESRNETKHLSVGKDIIQN

SEQ ID NO: 51 - protein, 41 aa, synthetic construct
PLTFRDVTIEFSLEEWQCLDTVQQNLYRDVMLENYRNLVFL

SEQ ID NO: 52 - protein, 80 aa, synthetic construct
PGAPGSLEMGPLTFRDVTIEFSLEEWQCLDTVQQNLYRDVMLENYRNLVFLGMAVFKPDLITCLKQGKEPWNMKRHEMVT

SEQ ID NO: 53 - protein, 41 aa, synthetic construct
YVTFEDVAVYFSQEEWRLLDDAQRLLYRNVMLENFTLLASL

SEQ ID NO: 54 - protein, 80 aa, synthetic construct
RDPAQVPVAADLLTDHEEGYVTFEDVAVYFSQEEWRLLDDAQRLLYRNVMLENFTLLASLGLASSKTHEITQLESWEEPF

SEQ ID NO: 55 - protein, 41 aa, synthetic construct
KVTFEDVAIDFTQEEWAMMDTSKRKLYRDVMLENISHLVSL

SEQ ID NO: 56 - protein, 80 aa, synthetic construct
HSLKKVTFEDVAIDFTQEEWAMMDTSKRKLYRDVMLENISHLVSLGYQISKSYIILQLEQGKELWREGREFLQDQNPDRE

SEQ ID NO: 57 - protein, 41 aa, synthetic construct
AVTFKDVAVVFSEEELQLLDLAQRKLYRDVMLENFRNVVSV

SEQ ID NO: 58 - protein, 80 aa, synthetic construct
TKSKEAVTFKDVAVVFSEEELQLLDLAQRKLYRDVMLENFRNVVSVGHQSTPDGLPQLEREEKLWMMKMATQRDNSSGAK

SEQ ID NO: 59 - protein, 41 aa, synthetic construct
QVTFSDVAIDFSHEEWACLDSAQRDLYKDVMVQNYENLVSV

SEQ ID NO: 60 - protein, 80 aa, synthetic construct
SQVTFSDVAIDFSHEEWACLDSAQRDLYKDVMVQNYENLVSVGLSVTKPYVIMLLEDGKEPWMMEKKLSKAYPFPLSHSV

SEQ ID NO: 61 - protein, 41 aa, synthetic construct
SLQFRDVAVEFSLEEWHCLDTAQQNLYRDVMLENYRHLVFL

SEQ ID NO: 62 - protein, 80 aa, synthetic construct
PGPLRSLEMESLQFRDVAVEFSLEEWHCLDTAQQNLYRDVMLENYRHLVFLGIIVSKPDLITCLEQGIKPLTMKRHEMIA

SEQ ID NO: 63 - protein, 40 aa, synthetic construct
SVTFEDVAVYFTQNQWASLDPAQRALYGEVMLENYANVAS

SEQ ID NO: 64 - protein, 80 aa, synthetic construct
LQTTWPQESVTFEDVAVYFTQNQWASLDPAQRALYGEVMLENYANVASLVAFPFPKPALISHLERGEAPWGPDPWDTEIL

SEQ ID NO: 65 - protein, 41 aa, synthetic construct
TVSFADVAVYFSPEEWGCLRPAQRALYRDVMQETYGHLGAL

SEQ ID NO: 66 - protein, 80 aa, synthetic construct
APLLAPRPGETRPGCRKPGTVSFADVAVYFSPEEWGCLRPAQRALYRDVMQETYGHLGALGFPGPKPALISWMEQESEAW

SEQ ID NO: 67 - protein, 41 aa, synthetic construct
SVSFKDVTVGFTQEEWQHLDPSQRALYRDVMLENYSNLVSV

SEQ ID NO: 68 - protein, 80 aa, synthetic construct
NKVEQKSQESVSFKDVTVGFTQEEWQHLDPSQRALYRDVMLENYSNLVSVGYCVHKPEVIFRLQQGEEPWKQEEEFPSQS

SEQ ID NO: 69 - protein, 41 aa, synthetic construct
LVTFEDVSMDFSQEEWELLEPAQKNLYREVMLENYRNVVSL

SEQ ID NO: 70 - protein, 80 aa, synthetic construct
CFSQEERMAAGYLPRWSQELVTFEDVSMDFSQEEWELLEPAQKNLYREVMLENYRNVVSLEALKNQCTDVGIKEGPLSPA

SEQ ID NO: 71 - protein, 41 aa, synthetic construct
SVAFEDVAVNFTQEEWALLDPSQKNLYREVMQETLRNLTSI

SEQ ID NO: 72 - protein, 80 aa, synthetic construct
DSVAFEDVAVNFTQEEWALLDPSQKNLYREVMQETLRNLTSIGKKWNNQYIEDEHQNPRRNLRRLIGERLSESKESHQHG

SEQ ID NO: 73 - protein, 41 aa, synthetic construct
QVNFEDVFVYFSQEEWVLLDEAQRLLYRDVMLENFALMASL

SEQ ID NO: 74 - protein, 80 aa, synthetic construct
MGPAQVPMNSEVIVDPIQGQVNFEDVFVYFSQEEWVLLDEAQRLLYRDVMLENFALMASLGHTSFMSHIVASLVMGSEPW

SEQ ID NO: 75 - protein, 41 aa, synthetic construct
AMTFKDVAVVFTEEELGLLDLAQRKLYRDVMLENFRNLLSV

SEQ ID NO: 76 - protein, 80 aa, synthetic construct
TTFKEAMTFKDVAVVFTEEELGLLDLAQRKLYRDVMLENFRNLLSVGHQAFHRDTFHFLREEKIWMMKTAIQREGNSGDK

SEQ ID NO: 77 - protein, 41 aa, synthetic construct
AVTFKDVIVDFTQEEWKQLDPGQRDLFRDVTLENYTHLVSI

SEQ ID NO: 78 - protein, 80 aa, synthetic construct
DSTLLQGGHNLLSSASFQEAVTFKDVIVDFTQEEWKQLDPGQRDLFRDVTLENYTHLVSIGLQVSKPDVISQLEQGTEPW

SEQ ID NO: 79 - protein, 41 aa, synthetic construct
SVCFEDVAMAFTQEEWEQLDLAQRTLYREVTLETWEHIVSL

SEQ ID NO: 80 - protein, 80 aa, synthetic construct
EARSMLVPPQASVCFEDVAMAFTQEEWEQLDLAQRTLYREVTLETWEHIVSLGLFLSKSDVISQLEQEEDLCRAEQEAPR

SEQ ID NO: 81 - protein, 41 aa, synthetic construct
SVVFEDVAVDFTLEEWALLDSAQRDLYRDVMLETFRNLASV

SEQ ID NO: 82 - protein, 80 aa, synthetic construct
DSVVFEDVAVDFTLEEWALLDSAQRDLYRDVMLETFRNLASVDDGTQFKANGSVSLQDMYGQEKSKEQTIPNFTGNNSCA

SEQ ID NO: 83 - protein, 41 aa, synthetic construct
LVTFRDVAIDFSQEEWECLDPAQRDLYVDVMLENYSNLVSL

SEQ ID NO: 84 - protein, 80 aa, synthetic construct
EESHGALISSCNSRTMTDGLVTFRDVAIDFSQEEWECLDPAQRDLYVDVMLENYSNLVSLDLESKTYETKKIFSENDIFE

SEQ ID NO: 85 - protein, 41 aa, synthetic construct
HVTFEDIAVYFSQEEWGLLDEAQRCLYHDVMLENFSLMASV

SEQ ID NO: 86 - protein, 80 aa, synthetic construct
VITPQIPMVTEEFVKPSQGHVTFEDIAVYFSQEEWGLLDEAQRCLYHDVMLENFSLMASVGCLHGIEAEEAPSEQTLSAQ

SEQ ID NO: 87 - protein, 41 aa, synthetic construct
SVTFEDVAVYFSWEEWDLLDEAQKHLYFDVMLENFALTSSL

SEQ ID NO: 88 - protein, 80 aa, synthetic construct
VQLRPQTRMATALRDPASGSVTFEDVAVYFSWEEWDLLDEAQKHLYFDVMLENFALTSSLGCWCGVEHEETPSEQRISGE

SEQ ID NO: 89 - protein, 41 aa, synthetic construct
PLQFRDVAIEFSLEEWHCLDAAQRNLYRDVMLENYRNLIFL

SEQ ID NO: 90 - protein, 80 aa, synthetic construct
GPLQFRDVAIEFSLEEWHCLDAAQRNLYRDVMLENYRNLIFLGIVVSKPNLITCLEQGKKPLTMKRHEMIAKPPVMYSHF

SEQ ID NO: 91 - protein, 41 aa, synthetic construct
SLTLEDVAVDFTWEEWQFLSPAQKDLYRDVMLENYSNLVAV

SEQ ID NO: 92 - protein, 80 aa, synthetic construct
MQAQESLTLEDVAVDFTWEEWQFLSPAQKDLYRDVMLENYSNLVAVGYQASKPDALSKLERGEETCTTEDEIYSRICSEI

SEQ ID NO: 93 - protein, 41 aa, synthetic construct
PLQFRDVAIEFSLEEWHCLDTAQRNLYRDVMLENYRNLVFL

SEQ ID NO: 94 - protein, 80 aa, synthetic construct
GPLQFRDVAIEFSLEEWHCLDTAQRNLYRDVMLENYRNLVFLGIVVSKPDLVTCLEQGKKPLTMERHEMIAKPPVMSSHF

SEQ ID NO: 95 - protein, 41 aa, synthetic construct
AVTFKDVAVAFTEEELGLLGPAQRKLYRDVMVENFRNLLSV

SEQ ID NO: 96 - protein, 80 aa, synthetic construct
NMFKEAVTFKDVAVAFTEEELGLLGPAQRKLYRDVMVENFRNLLSVGHPPFKQDVSPIERNEQLWIMTTATRRQGNLGEK

SEQ ID NO: 97 - protein, 41 aa, synthetic construct
ALTFRDVAIEFSLEEWQCLDTEQQNLYRNVMLDNYRNLVFL

SEQ ID NO: 98 - protein, 80 aa, synthetic construct
GALTFRDVAIEFSLEEWQCLDTEQQNLYRNVMLDNYRNLVFLGIAVSKPDLITCLEQEKEPWNLKTHDMVAKPPVICSHI

SEQ ID NO: 99 - protein, 41 aa, synthetic construct
SVSFRDVAIDFSREEWRHLDPSQRNLYRDVMLETYSHLLSV

SEQ ID NO: 100 - protein, 80 aa, synthetic construct
SPQKSSALAPEDHGSSYEGSVSFRDVAIDFSREEWRHLDPSQRNLYRDVMLETYSHLLSVGYQVPEAEVVMLEQGKEPWA

SEQ ID NO: 101 - protein, 41 aa, synthetic construct
LLTFRDVAIEFSPEEWKCLDPAQQNLYRDVMLENYRNLISL

SEQ ID NO: 102 - protein, 80 aa, synthetic construct
ELLTFRDVAIEFSPEEWKCLDPAQQNLYRDVMLENYRNLISLGVAISNPDLVIYLEQRKEPYKVKIHETVAKHPAVCSHF

SEQ ID NO: 103 - protein, 41 aa, synthetic construct
PLKFRDVAIEFSLEEWQCLDTIQQNLYRNVMLENYRNLVFL

SEQ ID NO: 104 - protein, 80 aa, synthetic construct
EPLKFRDVAIEFSLEEWQCLDTIQQNLYRNVMLENYRNLVFLGIVVSKPDLITCLEQEKEPWTRKRHRMVAEPPVICSHF

SEQ ID NO: 105 - protein, 41 aa, synthetic construct
PITFGDLAIYFSQEEWEWLSPIQKDLYEDVMLENYRNLVSL

SEQ ID NO: 106 - protein, 80 aa, synthetic construct
PSARGKSKSKAPITFGDLAIYFSQEEWEWLSPIQKDLYEDVMLENYRNLVSLGLSFRRPNVITLLEKGKAPWMVEPVRRR

SEQ ID NO: 107 - protein, 41 aa, synthetic construct
SLTLEDVAVDFTWEEWQFLSPAQKDLYRDVMLENYSNLVSV

SEQ ID NO: 108 - protein, 80 aa, synthetic construct
TKAQESLTLEDVAVDFTWEEWQFLSPAQKDLYRDVMLENYSNLVSVGYQAGKPDALTKLEQGEPLWTLEDEIHSPAHPEI

SEQ ID NO: 109 - protein, 41 aa, synthetic construct
SVTFTDVAIDFSQDEWEWLNLAQRSLYKKVMLENYRNLVSV

SEQ ID NO: 110 - protein, 80 aa, synthetic construct
SQEEVEVAGIKLCKAMSLGSVTFTDVAIDFSQDEWEWLNLAQRSLYKKVMLENYRNLVSVGLCISKPDVISLLEQEKDPW

SEQ ID NO: 111 - protein, 41 aa, synthetic construct
SVSFKDVTVDFSRDEWQQLDLAQKSLYREVMLENYFNLISV

SEQ ID NO: 112 - protein, 80 aa, synthetic construct
TKSLESVSFKDVTVDFSRDEWQQLDLAQKSLYREVMLENYFNLISVGCQVPKPEVIFSLEQEEPCMLDGEIPSQSRPDGD

SEQ ID NO: 113 - protein, 41 aa, synthetic construct
TLTFRDVAIEFSLEEWECLNPAQQNLYMNVMLENYKNLVFL

SEQ ID NO: 114 - protein, 80 aa, synthetic construct
SGCPGAERNLLVYSYFEKETLTFRDVAIEFSLEEWECLNPAQQNLYMNVMLENYKNLVFLGVAVSKQDPVTCLEQEKEPW

SEQ ID NO: 115 - protein, 41 aa, synthetic construct
SVSFKDVTVDFTQEEWQQLDPAQKALYRDVMLENYCHFVSV

SEQ ID NO: 116 - protein, 80 aa, synthetic construct
PLQGSVSFKDVTVDFTQEEWQQLDPAQKALYRDVMLENYCHFVSVGFHMAKPDMIRKLEQGEELWTQRIFPSYSYLEEDG

SEQ ID NO: 117 - protein, 41 aa, synthetic construct
LLTFRDVAIEFSLEEWQHLDIAQQNLYRNVMLENYRNLAFL

SEQ ID NO: 118 - protein, 80 aa, synthetic construct
PGPPRSLEMGLLTFRDVAIEFSLEEWQHLDIAQQNLYRNVMLENYRNLAFLGIAVSKPDLITCLEQGKEPWNMKRHEMVD

SEQ ID NO: 119 - protein, 41 aa, synthetic construct
SVAFEDVAVNFTQEEWALLDPSQKNLYRDVMQETFRNLASI

SEQ ID NO: 120 - protein, 80 aa, synthetic construct
SGHPGSWEMNSVAFEDVAVNFTQEEWALLDPSQKNLYRDVMQETFRNLASIGNKGEDQSIEDQYKNSSRNLRHIISHSGN

SEQ ID NO: 121 - protein, 40 aa, synthetic construct
SITFGDVAIDFSHQEWEYLSLVQKTLYQEVMMENYDNLVS

SEQ ID NO: 122 - protein, 80 aa, synthetic construct
SYGSITFGDVAIDFSHQEWEYLSLVQKTLYQEVMMENYDNLVSLAGHSVSKPDLITLLEQGKEPWMIVREETRGECTDLD

SEQ ID NO: 123 - protein, 41 aa, synthetic construct
SVTFQDVAVDFTEKEWPLLDSSQRKLYKDVMLENYSNLTSL

SEQ ID NO: 124 - protein, 80 aa, synthetic construct
DLFVCSGLEPHTPSVGSQESVTFQDVAVDFTEKEWPLLDSSQRKLYKDVMLENYSNLTSLGYQVGKPSLISHLEQEEEPR

SEQ ID NO: 125 - protein, 40 aa, synthetic construct
PVTFEDVAVYFTQNEWASLDSVQRALYREVMLENYANVAS

SEQ ID NO: 126 - protein, 80 aa, synthetic construct
FQTAWRQEPVTFEDVAVYFTQNEWASLDSVQRALYREVMLENYANVASLAFPFTTPVLVSQLEQGELPWGLDPWEPMGRE

SEQ ID NO: 127 - protein, 41 aa, synthetic construct
LLTFRDVAIEFSPEEWKCLDPDQQNLYRDVMLENYRNLVSL

SEQ ID NO: 128 - protein, 80 aa, synthetic construct
ELLTFRDVAIEFSPEEWKCLDPDQQNLYRDVMLENYRNLVSLGVAISNPDLVTCLEQRKEPYNVKIHKIVARPPAMCSHF

SEQ ID NO: 129 - protein, 41 aa, synthetic construct
LVMFEDVTVYFSREEWGLLNVTQKGLYRDVMLENFALVSSL

SEQ ID NO: 130 - protein, 80 aa, synthetic construct
AGEAEAQLDPSLQGLVMFEDVTVYFSREEWGLLNVTQKGLYRDVMLENFALVSSLGLAPSRSPVFTQLEDDEQSWVPSWV

SEQ ID NO: 131 - protein, 41 aa, synthetic construct
LVTFRDVAIDFSQKEWECLDTTQRKLYRDVMLENYNNLVSL

SEQ ID NO: 132 - protein, 80 aa, synthetic construct
AHALVTFRDVAIDFSQKEWECLDTTQRKLYRDVMLENYNNLVSLGYSGSKPDVITLLEQGKEPCVVARDVTGRQCPGLLS

SEQ ID NO: 133 - protein, 41 aa, synthetic construct
LLTFEDVAVYFSEEEWQLLNPLEKTLYNDVMQDIYETVISL

SEQ ID NO: 134 - protein, 80 aa, synthetic construct
KRIKHWKMASKLILPESLSLLTFEDVAVYFSEEEWQLLNPLEKTLYNDVMQDIYETVISLGLKLKNDTGNDHPISVSTSE

SEQ ID NO: 135 - protein, 41 aa, synthetic construct
SVVFEDVAVDFTLEEWALLDSAQRDLYRDVMLETFQNLASV

SEQ ID NO: 136 - protein, 80 aa, synthetic construct
DSVVFEDVAVDFTLEEWALLDSAQRDLYRDVMLETFQNLASVDDETQFKASGSVSQQDIYGEKIPKESKIATFTRNVSWA

SEQ ID NO: 137 - protein, 41 aa, synthetic construct
SVSFQDVTVEFTREEWQHLGPVERTLYRDVMLENYSHLISV

SEQ ID NO: 138 - protein, 80 aa, synthetic construct
NMSQASVSFQDVTVEFTREEWQHLGPVERTLYRDVMLENYSHLISVGYCITKPKVISKLEKGEEPWSLEDEFLNQRYPGY

SEQ ID NO: 139 - protein, 41 aa, synthetic construct
SVTFQDVAVDFTAEEWQLLDCAERTLYWDVMLENYRNLISV

SEQ ID NO: 140 - protein, 80 aa, synthetic construct
ISFQESVTFQDVAVDFTAEEWQLLDCAERTLYWDVMLENYRNLISVGCPITKTKVILKVEQGQEPWMVEGANPHESSPES

SEQ ID NO: 141 - protein, 41 aa, synthetic construct
PVSFKDVAVDFTQEEWQQLDPDEKITYRDVMLENYSHLVSV

SEQ ID NO: 142 - protein, 80 aa, synthetic construct
NTLQGPVSFKDVAVDFTQEEWQQLDPDEKITYRDVMLENYSHLVSVGYDTTKPNVIIKLEQGEEPWIMGGEFPCQHSPEA

SEQ ID NO: 143 - protein, 41 aa, synthetic construct
PVMFRDVSIDFSQEEWECLDADQMNLYKEVMLENFSNLVSV

SEQ ID NO: 144 - protein, 80 aa, synthetic construct
HPEEEERMHDELLQAVSKGPVMFRDVSIDFSQEEWECLDADQMNLYKEVMLENFSNLVSVGLSNSKPAVISLLEQGKEPW

SEQ ID NO: 145 - protein, 39 aa, synthetic construct
LFRDVAIVFSQEEWQWLAPAQRDLYRDVMLETYSNLVSL

SEQ ID NO: 146 - protein, 80 aa, synthetic construct
SLGSELFRDVAIVFSQEEWQWLAPAQRDLYRDVMLETYSNLVSLGLAVSKPDVISFLEQGKEPWMVERVVSGGLCPVLES

SEQ ID NO: 147 - protein, 40 aa, synthetic construct
MVTFKDVAVVFTEEELGLLDSVQRKLYRDVMLENFRNLLL

SEQ ID NO: 148 - protein, 80 aa, synthetic construct
TKFQEMVTFKDVAVVFTEEELGLLDSVQRKLYRDVMLENFRNLLLVAHQPFKPDLISQLEREEKLLMVETETPRDGCSGR

SEQ ID NO: 149 - protein, 41 aa, synthetic construct
LLTFRDIAIEFSLAEWQCLDHAQQNLYRDVMLENYRNLVSL

SEQ ID NO: 150 - protein, 80 aa, synthetic construct
AKRPGPPGSREMGLLTFRDIAIEFSLAEWQCLDHAQQNLYRDVMLENYRNLVSLGIAVSKPDLITCLEQNKEPQNIKRNE

SEQ ID NO: 151 - protein, 41 aa, synthetic construct
FVAFRDVAVYFTQEEWRLLSPAQRTLHREVMLETYNHLVSL

SEQ ID NO: 152 - protein, 80 aa, synthetic construct
TCMVHRQTMSCSGAGGITAFVAFRDVAVYFTQEEWRLLSPAQRTLHREVMLETYNHLVSLEIPSSKPKLIAQLERGEAPW

SEQ ID NO: 153 - protein, 41 aa, synthetic construct
SITLEDVAVDFTWEEWQLLGAAQKDLYRDVMLENYSNLVAV

SEQ ID NO: 154 - protein, 80 aa, synthetic construct
IQAQESITLEDVAVDFTWEEWQLLGAAQKDLYRDVMLENYSNLVAVGYQASKPDALFKLEQGEQLWTIEDGIHSGACSDI

SEQ ID NO: 155 - protein, 41 aa, synthetic construct
HLTFRDVAIEFSQAEWKCLDPAQRALYKDVMLENYRNLVSL

SEQ ID NO: 156 - protein, 80 aa, synthetic construct
AQKRRKRKAKESGMALPQGHLTFRDVAIEFSQAEWKCLDPAQRALYKDVMLENYRNLVSLGISLPDLNINSMLEQRREPW

SEQ ID NO: 157 - protein, 40 aa, synthetic construct
CVTFEDVAIYFSQEEWGLLDEAQRLLYRDVMLENFALITA

SEQ ID NO: 158 - protein, 80 aa, synthetic construct
DSTSVPVTAEAKLMGFTQGCVTFEDVAIYFSQEEWGLLDEAQRLLYRDVMLENFALITALVCWHGMEDEETPEQSVSVEG

SEQ ID NO: 159 - protein, 41 aa, synthetic construct
PLTFRDVKIEFSLEEWQCLDTAQRNLYRDVMLENYRNLVFL

SEQ ID NO: 160 - protein, 80 aa, synthetic construct
GPLTFRDVKIEFSLEEWQCLDTAQRNLYRDVMLENYRNLVFLGIAVSKPDLITWLEQGKEPWNLKRHEMVDKTPVMCSHF

SEQ ID NO: 161 - protein, 40 aa, synthetic construct
PLTFRDVAIEFSLEEWQCLDSAQQGLYRKVMLENYRNLVF

SEQ ID NO: 162 - protein, 80 aa, synthetic construct
SGCPGAERSLLVQSYFEKGPLTFRDVAIEFSLEEWQCLDSAQQGLYRKVMLENYRNLVFLAGIALTKPDLITCLEQGKEP

SEQ ID NO: 163 - protein, 41 aa, synthetic construct
VLTFRDVAVEFSPEEWECLDSAQQRLYRDVMLENYGNLVSL

SEQ ID NO: 164 - protein, 80 aa, synthetic construct
GVLTFRDVAVEFSPEEWECLDSAQQRLYRDVMLENYGNLVSLGLAIFKPDLMTCLEQRKEPWKVKRQEAVAKHPAGSFHF

SEQ ID NO: 165 - protein, 41 aa, synthetic construct
SVSFKDVAVDFTQEEWGQLDSPQRALYRDVMLENYQNLLAL

SEQ ID NO: 166 - protein, 80 aa, synthetic construct
KENLEDISGWGLPEARSKESVSFKDVAVDFTQEEWGQLDSPQRALYRDVMLENYQNLLALGPPLHKPDVISHLERGEEPW

SEQ ID NO: 167 - protein, 52 aa, synthetic construct
EPERIIGATDSSGELMFLMKWKNSDEADLVPAKEANVKCPQVVISFYEERLT

SEQ ID NO: 168 - protein, 80 aa, synthetic construct
EESEKPRGFARGLEPERIIGATDSSGELMFLMKWKNSDEADLVPAKEANVKCPQVVISFYEERLTWHSYPSEDDDKKDDK

SEQ ID NO: 169 - protein, 40 aa, synthetic construct
SVALEDVAVNFTREEWALLGPCQKNLYKDVMQETIRNLDC

SEQ ID NO: 170 - protein, 80 aa, synthetic construct
ASVALEDVAVNFTREEWALLGPCQKNLYKDVMQETIRNLDCVVMKWKDQNIEDQYRYPRKNLRCRMLERFVESKDGTQCG

SEQ ID NO: 171 - protein, 41 aa, synthetic construct
LLTFRDVAIEFSLEEWKCLDLAQQNLYRDVMLENYRNLFSV

SEQ ID NO: 172 - protein, 80 aa, synthetic construct
TLLTFRDVAIEFSLEEWKCLDLAQQNLYRDVMLENYRNLFSVGLTVCKPGLITCLEQRKEPWNVKRQEAADGHPEMGFHH

SEQ ID NO: 173 - protein, 41 aa, synthetic construct
FVAFEDVAIYFSQEEWELLDEMQRLLYRDVMLENFAVMASL

SEQ ID NO: 174 - protein, 80 aa, synthetic construct
AAALRAPTQQVFVAFEDVAIYFSQEEWELLDEMQRLLYRDVMLENFAVMASLGCWCGAVDEGTPSAESVSVEELSQGRTP

SEQ ID NO: 175 - protein, 41 aa, synthetic construct
SVSFQDVTVEFSQEEWQHMGPVERTLYRDVMLENYSHLVSV

SEQ ID NO: 176 - protein, 80 aa, synthetic construct
NTFQASVSFQDVTVEFSQEEWQHMGPVERTLYRDVMLENYSHLVSVGYCFTKPELIFTLEQGEDPWLLEKEKGFLSRNSP

SEQ ID NO: 177 - protein, 41 aa, synthetic construct
SVAFEDVSVSFSQEEWALLAPSQKKLYRDVMQETFKNLASI

SEQ ID NO: 178 - protein, 80 aa, synthetic construct
DSVAFEDVSVSFSQEEWALLAPSQKKLYRDVMQETFKNLASIGEKWEDPNVEDQHKNQGRNLRSHTGERLCEGKEGSQCA

SEQ ID NO: 179 - protein, 41 aa, synthetic construct
LVTFADVAIDFSQEEWACLNSAQRDLYWDVMLENYSNLVSL

SEQ ID NO: 180 - protein, 80 aa, synthetic construct
AQGLVTFADVAIDFSQEEWACLNSAQRDLYWDVMLENYSNLVSLDLESAYENKSLPTEKNIHEIRASKRNSDRRSKSLGR

SEQ ID NO: 181 - protein, 41 aa, synthetic construct
PVTFRDVAVFFSQDEWLHLDSAQRALYREVMLENYSSLVSL

SEQ ID NO: 182 - protein, 80 aa, synthetic construct
AVDLLSAQEPVTFRDVAVFFSQDEWLHLDSAQRALYREVMLENYSSLVSLGIPFSMPKLIHQLQQGEDPCMVEREVPSDT

SEQ ID NO: 183 - protein, 41 aa, synthetic construct
SVSFEDVAVDFTRQEWHRLDPAQRTMHKDVMLETYSNLASV

SEQ ID NO: 184 - protein, 80 aa, synthetic construct
SPQRFPALIPGEPGRSFEGSVSFEDVAVDFTRQEWHRLDPAQRTMHKDVMLETYSNLASVGLCVAKPEMIFKLERGEELW

SEQ ID NO: 185 - protein, 41 aa, synthetic construct
VLTFRDVAVEFSPEEWECLDSAQQRLYRDVMLENYGNLFSL

SEQ ID NO: 186 - protein, 80 aa, synthetic construct
RVLTFRDVAVEFSPEEWECLDSAQQRLYRDVMLENYGNLFSLGLAIFKPDLITYLEQRKEPWNARRQKTVAKHPAGSLHF

SEQ ID NO: 187 - protein, 41 aa, synthetic construct
LVTFKDVAVTFTREEWRQLDLAQRTLYREVMLETCGLLVSL

SEQ ID NO: 188 - protein, 80 aa, synthetic construct
AETKDAAQMLVTFKDVAVTFTREEWRQLDLAQRTLYREVMLETCGLLVSLGHRVPKPELVHLLEHGQELWIVKRGLSHAT

SEQ ID NO: 189 - protein, 41 aa, synthetic construct
PVSFKDVVVGFTQEEWHRLSPAQRALYRDVMLETYSNLVSV

SEQ ID NO: 190 - protein, 80 aa, synthetic construct
IEYQIPVSFKDVVVGFTQEEWHRLSPAQRALYRDVMLETYSNLVSVGYEGTKPDVILRLEQEEAPWIGEAACPGCHCWED

SEQ ID NO: 191 - protein, 41 aa, synthetic construct
AVTFKDVAVAFTEEELGLLDSAQRKLYRDVMLENFRNLVSV

SEQ ID NO: 192 - protein, 80 aa, synthetic construct
TKFQEAVTFKDVAVAFTEEELGLLDSAQRKLYRDVMLENFRNLVSVGHQSFKPDMISQLEREEKLWMKELQTQRGKHSGD

SEQ ID NO: 193 - protein, 41 aa, synthetic construct
PVTFRDVAVDFTQEEWGQLDPTQRILYRDVMLETFGHLLSI

SEQ ID NO: 194 - protein, 80 aa, synthetic construct
DEGVAGVMSVGPPAARLQEPVTFRDVAVDFTQEEWGQLDPTQRILYRDVMLETFGHLLSIGPELPKPEVISQLEQGTELW

SEQ ID NO: 195 - protein, 41 aa, synthetic construct
PLTFMDVAIEFSVEEWQCLDTAQQNLYRNVMLENYRNLVFL

SEQ ID NO: 196 - protein, 80 aa, synthetic construct
GPLTFMDVAIEFSVEEWQCLDTAQQNLYRNVMLENYRNLVFLGIAVSKPDLITCLEQGKEPWNMERHEMVAKPPGMCCYF

SEQ ID NO: 197 - protein, 41 aa, synthetic construct
LVTFRDVAIDFSRQEWEYLDPNQRDLYRDVMLENYRNLVSL

SEQ ID NO: 198 - protein, 80 aa, synthetic construct
HQVGLIRSYNSKTMTCFQELVTFRDVAIDFSRQEWEYLDPNQRDLYRDVMLENYRNLVSLGGHSISKPVVVDLLERGKEP

SEQ ID NO: 199 - protein, 41 aa, synthetic construct
SLSFEDVAVGFTREEWQFLDQSQKVLYKEVMLENYINLVSI

SEQ ID NO: 200 - protein, 80 aa, synthetic construct
NATIVMSVRREQGSSSGEGSLSFEDVAVGFTREEWQFLDQSQKVLYKEVMLENYINLVSIGYRGTKPDSLFKLEQGEPPG

SEQ ID NO: 201 - protein, 41 aa, synthetic construct
LLSFEDVAMYFTREEWGHLNWGQKDLYRDVMLENYRNMVLL

SEQ ID NO: 202 - protein, 80 aa, synthetic construct
FPPARGKELLSFEDVAMYFTREEWGHLNWGQKDLYRDVMLENYRNMVLLGFQFPKPEMICQLENWDEQWILDLPRTGNRK

SEQ ID NO: 203 - protein, 41 aa, synthetic construct
LLTFKDVAIEFSPEEWKCLDTSQQNLYRDVMLENYRNLVSL

SEQ ID NO: 204 - protein, 80 aa, synthetic construct
ELLTFKDVAIEFSPEEWKCLDTSQQNLYRDVMLENYRNLVSLGVSISNPDLVTSLEQRKEPYNLKIHETAARPPAVCSHF

SEQ ID NO: 205 - protein, 41 aa, synthetic construct
LVSFKDVAVDFTQEEWQQLDPSQRTLYRDVMLENYSHLVSM

SEQ ID NO: 206 - protein, 80 aa, synthetic construct
MKSQGLVSFKDVAVDFTQEEWQQLDPSQRTLYRDVMLENYSHLVSMGYPVSKPDVISKLEQGEEPWIIKGDISNWIYPDE

SEQ ID NO: 207 - protein, 41 aa, synthetic construct
SVMFSDVSIDFSQEEWDCLDPVQRDLYRDVMLENYGNLVSM

SEQ ID NO: 208 - protein, 80 aa, synthetic construct
AEGSVMFSDVSIDFSQEEWDCLDPVQRDLYRDVMLENYGNLVSMGLYTPKPQVISLLEQGKEPWMVGRELTRGLCSDLES

SEQ ID NO: 209 - protein, 41 aa, synthetic construct
PLTFTDVAIEFSLEEWQCLDTAQQNLYRNVMLENYRNLVFL

SEQ ID NO: 210 - protein, 80 aa, synthetic construct
GPLTFTDVAIEFSLEEWQCLDTAQQNLYRNVMLENYRNLVFLGIAVSKPDLITCLEKEKEPCKMKRHEMVDEPPVVCSHF

SEQ ID NO: 211 - protein, 41 aa, synthetic construct
LFTFKDVAIEFSQEEWECLDPAQRALYRDVMLENYRNLLSL

SEQ ID NO: 212 - protein, 80 aa, synthetic construct
ALSQGLFTFKDVAIEFSQEEWECLDPAQRALYRDVMLENYRNLLSLDEDNIPPEDDISVGFTSKGLSPKENNKEELYHLV

SEQ ID NO: 213 - protein, 41 aa, synthetic construct
LVMFQDLSIDFSQEEWECLDAAQKDLYRDVMMENYSSLVSL

SEQ ID NO: 214 - protein, 80 aa, synthetic construct
NMEGLVMFQDLSIDFSQEEWECLDAAQKDLYRDVMMENYSSLVSLGLSIPKPDVISLLEQGKEPWMVSRDVLGGWCRDSE

SEQ ID NO: 215 - protein, 41 aa, synthetic construct
SVTFKDVAILFTQEEWGQLSPAQRALYRDVMLENYSNLVSL

SEQ ID NO: 216 - protein, 80 aa, synthetic construct
AVSHLPTMVQESVTFKDVAILFTQEEWGQLSPAQRALYRDVMLENYSNLVSLGLLGPKPDTFSQLEKREVWMPEDTPGGF

SEQ ID NO: 217 - protein, 41 aa, synthetic construct
PLTIRDVTVEFSLEEWHCLDTAQQNLYRDVMLENYRNLVFL

SEQ ID NO: 218 - protein, 80 aa, synthetic construct
GPLTIRDVTVEFSLEEWHCLDTAQQNLYRDVMLENYRNLVFLGIAVSKPDLITCLEQGKEPCNMKRHEMVAKPPVMCSHI

SEQ ID NO: 219 - protein, 41 aa, synthetic construct
SVTFDDVAVTFTKEEWGQLDLAQRTLYQEVMLENCGLLVSL

SEQ ID NO: 220 - protein, 80 aa, synthetic construct
AAAVLTDRAQVSVTFDDVAVTFTKEEWGQLDLAQRTLYQEVMLENCGLLVSLGCPVPKAELICHLEHGQEPWTRKEDLSQ

SEQ ID NO: 221 - protein, 41 aa, synthetic construct
SVMFSDVSIDFSPEEWEYLDLEQKDLYRDVMLENYSNLVSL

SEQ ID NO: 222 - protein, 80 aa, synthetic construct
ALRSVMFSDVSIDFSPEEWEYLDLEQKDLYRDVMLENYSNLVSLGCFISKPDVISSLEQGKEPWKVVRKGRRQYPDLETK

SEQ ID NO: 223 - protein, 41 aa, synthetic construct
SVTFRDVAIDFSQEEWEFLDPAQRDLYRDVMWENYSNFISL

SEQ ID NO: 224 - protein, 80 aa, synthetic construct
AHGSVTFRDVAIDFSQEEWEFLDPAQRDLYRDVMWENYSNFISLGPSISKPDVITLLDEERKEPGMVVREGTRRYCPDLE

SEQ ID NO: 225 - protein, 41 aa, synthetic construct
PLTFGDVAVAFTRIEWRHLDAAQRALYRDVMLENYGNLVSV

SEQ ID NO: 226 - protein, 80 aa, synthetic construct
APRAQIQGPLTFGDVAVAFTRIEWRHLDAAQRALYRDVMLENYGNLVSVGLLSSKPKLITQLEQGAEPWTEVREAPSGTH

SEQ ID NO: 227 - protein, 41 aa, synthetic construct
PLQFRDVAIEFSLEEWHCLDTAQRNLYRNVMLENYRNLVFL

SEQ ID NO: 228 - protein, 80 aa, synthetic construct
GPLQFRDVAIEFSLEEWHCLDTAQRNLYRNVMLENYRNLVFLGIVVSKPDLITCLEQGKKPLTMKKHEMVANPSVTCSHF

SEQ ID NO: 229 - protein, 41 aa, synthetic construct
SVAFEDVAVNFTHEEWALLGPSQKNLYRDVMRETIRNLNCI

SEQ ID NO: 230 - protein, 80 aa, synthetic construct
TLPRGQPEVLEWGLPKDQDSVAFEDVAVNFTHEEWALLGPSQKNLYRDVMRETIRNLNCIGMKWENQNIDDQHQNLRRNP

SEQ ID NO: 231 - protein, 41 aa, synthetic construct
SMTFEDIIVDFTQEEWALLDTSQRKLFQDVMLENISHLVSI

SEQ ID NO: 232 - protein, 80 aa, synthetic construct
PSPDSMTFEDIIVDFTQEEWALLDTSQRKLFQDVMLENISHLVSIGKQLCKSVVLSQLEQVEKLSTQRISLLQGREVGIK

SEQ ID NO: 233 - protein, 41 aa, synthetic construct
LVTFRDVAIEFSLEEWKCLEPAQRDLYREVTLENFGHLASL

SEQ ID NO: 234 - protein, 80 aa, synthetic construct
EESREIRAGQIVLKAMAQGLVTFRDVAIEFSLEEWKCLEPAQRDLYREVTLENFGHLASLGLSISKPDVVSLLEQGKEPW

SEQ ID NO: 235 - protein, 41 aa, synthetic construct
SVTFEDVAVTFTQEEWGQLDAAQRTLYQEVMLETCGLLMSL

SEQ ID NO: 236 - protein, 80 aa, synthetic construct
AASAQVSVTFEDVAVTFTQEEWGQLDAAQRTLYQEVMLETCGLLMSLGCPLFKPELIYQLDHRQELWMATKDLSQSSYPG

SEQ ID NO: 237 - protein, 41 aa, synthetic construct
LLTFKDISIDFTQEEWGQLAPAHQNLYREVMLENYSNLVSV

SEQ ID NO: 238 - protein, 80 aa, synthetic construct
RESLEDEVTPGLPTAESQELLTFKDISIDFTQEEWGQLAPAHQNLYREVMLENYSNLVSVGYQLSKPSVISQLEKGEEPW

SEQ ID NO: 239 - protein, 70 aa, synthetic construct
IKLKVIGQDSSEIHFKVKMTTHLKKLKESYCQRQGVPMNSLRFLFEGQRIADNHTPKELGMEEEDVIEVY

SEQ ID NO: 240 - protein, 80 aa, synthetic construct
EGEYIKLKVIGQDSSEIHFKVKMTTHLKKLKESYCQRQGVPMNSLRFLFEGQRIADNHTPKELGMEEEDVIEVYQEQTGG

SEQ ID NO: 241 - protein, 41 aa, synthetic construct
PVSFKDVAVDFTQEEWQQLDPEQKITYRDVMLENYSNLVSV

SEQ ID NO: 242 - protein, 80 aa, synthetic construct
NKSLGPVSFKDVAVDFTQEEWQQLDPEQKITYRDVMLENYSNLVSVGYHIIKPDVISKLEQGEEPWIVEGEFLLQSYPDE

SEQ ID NO: 243 - protein, 41 aa, synthetic construct
LMAFRDVAVAFTQKEWKLLSSAQRTLYREVMLENYSHLVSL

SEQ ID NO: 244 - protein, 80 aa, synthetic construct
SPGLLTTRKEALMAFRDVAVAFTQKEWKLLSSAQRTLYREVMLENYSHLVSLGIAFSKPKLIEQLEQGDEPWREENEHLL

SEQ ID NO: 245 - protein, 41 aa, synthetic construct
SVAFEDVAVTFTQEEWALLDPSQKNLCRDVMQETFRNLASI

SEQ ID NO: 246 - protein, 80 aa, synthetic construct
MFQDSVAFEDVAVTFTQEEWALLDPSQKNLCRDVMQETFRNLASIGKKWKPQNIYVEYENLRRNLRIVGERLFESKEGHQ

SEQ ID NO: 247 - protein, 70 aa, synthetic construct
INLKVAGQDGSVVQFKIKRHTPLSKLMKAYCERQGLSMRQIRFRFDGQPINETDTPAQLEMEDEDTIDVF

SEQ ID NO: 248 - protein, 80 aa, synthetic construct
ENDHINLKVAGQDGSVVQFKIKRHTPLSKLMKAYCERQGLSMRQIRFRFDGQPINETDTPAQLEMEDEDTIDVFQQQTGG

SEQ ID NO: 249 - protein, 41 aa, synthetic construct
VLTFRDVALEFSLEEWQCLDTAQQNLYRNVMLENYRNLVFV

SEQ ID NO: 250 - protein, 80 aa, synthetic construct
PGPLGSLEMGVLTFRDVALEFSLEEWQCLDTAQQNLYRNVMLENYRNLVFVGIAASKPDLITCLEQGKEPWNVKRHEMVT

SEQ ID NO: 251 - protein, 41 aa, synthetic construct
SVSFEDVTVDFSREEWQQLDPAQRCLYRDVMLELYSHLFAV

SEQ ID NO: 252 - protein, 80 aa, synthetic construct
LSQKPQVLGPEKQDGSCEASVSFEDVTVDFSREEWQQLDPAQRCLYRDVMLELYSHLFAVGYHIPNPEVIFRMLKEKEPR

SEQ ID NO: 253 - protein, 41 aa, synthetic construct
QVTFRDVAIEFSQEEWTCLDPAQRTLYRDVMLENYRNLASL

SEQ ID NO: 254 - protein, 80 aa, synthetic construct
ALTQGQVTFRDVAIEFSQEEWTCLDPAQRTLYRDVMLENYRNLASLGISCFDLSIISMLEQGKEPFTLESQVQIAGNPDG

SEQ ID NO: 255 - protein, 41 aa, synthetic construct
PVTLKDVIVEFTKEEWKLLTPAQRTLYKDVMLENYSHLVSV

SEQ ID NO: 256 - protein, 80 aa, synthetic construct
NKFQGPVTLKDVIVEFTKEEWKLLTPAQRTLYKDVMLENYSHLVSVGYHVNKPNAVFKLKQGKEPWILEVEFPHRGFPED

SEQ ID NO: 257 - protein, 40 aa, synthetic construct
LLTFRDVAIEFSPEEWKCLDPAQQNLYRDVMLENYRNLVS

SEQ ID NO: 258 - protein, 80 aa, synthetic construct
ELLTFRDVAIEFSPEEWKCLDPAQQNLYRDVMLENYRNLVSLAVYSYYNQGILPEQGIQDSFKKATLGRYGSCGLENICL

SEQ ID NO: 259 - protein, 41 aa, synthetic construct
SVSFRDVAIDFSREEWRHLDLSQRNLYRDVMLETYSHLLSV

SEQ ID NO: 260 - protein, 80 aa, synthetic construct
SPQKSSALAPEDHGSSYEGSVSFRDVAIDFSREEWRHLDLSQRNLYRDVMLETYSHLLSVGYQVPKPEVVMLEQGKEPWA

SEQ ID NO: 261 - protein, 41 aa, synthetic construct
RVTFEDVTVNFTQGEWQRLNPEQRNLYRDVMLENYSNLVSV

SEQ ID NO: 262 - protein, 80 aa, synthetic construct
NNSQGRVTFEDVTVNFTQGEWQRLNPEQRNLYRDVMLENYSNLVSVGQGETTKPDVILRLEQGKEPWLEEEEVLGSGRAE

SEQ ID NO: 263 - protein, 41 aa, synthetic construct
AVVFEDVAVYFTRIEWSCLAPDQQALYRDVMLENYGNLASL

SEQ ID NO: 264 - protein, 80 aa, synthetic construct
AMALPMPGPQEAVVFEDVAVYFTRIEWSCLAPDQQALYRDVMLENYGNLASLGFLVAKPALISLLEQGEEPGALILQVAE

SEQ ID NO: 265 - protein, 40 aa, synthetic construct
LVTFEDVAVDFTQEEWTLLDQAQRDLYRDVMLENYKNLII

SEQ ID NO: 266 - protein, 80 aa, synthetic construct
DSSQHLVTFEDVAVDFTQEEWTLLDQAQRDLYRDVMLENYKNLIILAGSELFKRSLMSGLEQMEELRTGVTGVLQELDLQ

SEQ ID NO: 267 - protein, 41 aa, synthetic construct
AVTFKDVAVFFTEEELGLLDPAQRKLYQDVMLENFTNLLSV

SEQ ID NO: 268 - protein, 80 aa, synthetic construct
TTFKEAVTFKDVAVFFTEEELGLLDPAQRKLYQDVMLENFTNLLSVGHQPFHPFHFLREEKFWMMETATQREGNSGGKTI

SEQ ID NO: 269 - protein, 41 aa, synthetic construct
PLQFRDVAIEFSLEEWHCLDMAQRNLYRDVMLENYRNLVFL

SEQ ID NO: 270 - protein, 80 aa, synthetic construct
GPLQFRDVAIEFSLEEWHCLDMAQRNLYRDVMLENYRNLVFLGIVVSKPDLITHLEQGKKPSTMQRHEMVANPSVLCSHF

SEQ ID NO: 271 - protein, 41 aa, synthetic construct
SVSFTDVTVDFTQEEWEQLDPSQRILYMDVMLENYSNLLSV

SEQ ID NO: 272 - protein, 80 aa, synthetic construct
NKSQGSVSFTDVTVDFTQEEWEQLDPSQRILYMDVMLENYSNLLSVEVWKADDQMERDHRNPDEQARQFLILKNQTPIEE

SEQ ID NO: 273 - protein, 41 aa, synthetic construct
SLTFQDVAVDFTREEWDQLYPAQKNLYRDVMLENYRNLVAL

SEQ ID NO: 274 - protein, 80 aa, synthetic construct
EEEEMNDGSQMVRSQESLTFQDVAVDFTREEWDQLYPAQKNLYRDVMLENYRNLVALGYQLCKPEVIAQLELEEEWVIER

SEQ ID NO: 275 - protein, 41 aa, synthetic construct
RLTFRDVAIEFSLEEWKCLNPAQRALYRAVMLENYRNLEFV

SEQ ID NO: 276 - protein, 80 aa, synthetic construct
EEATKKSKEKEPGMALPQGRLTFRDVAIEFSLEEWKCLNPAQRALYRAVMLENYRNLEFVDSSLKSMMEFSSTRHSITGE

SEQ ID NO: 277 - protein, 41 aa, synthetic construct
SVTFDDVAVDFTQEEWTLLDSTQRSLYSDVMLENYKNLATV

SEQ ID NO: 278 - protein, 80 aa, synthetic construct
EKTPAGRIVADCLTDCYQDSVTFDDVAVDFTQEEWTLLDSTQRSLYSDVMLENYKNLATVGGQIIKPSLISWLEQEESRT

SEQ ID NO: 279 - protein, 41 aa, synthetic construct
LLTFRDVAIEFSQEEWKCLDPAQRTLYRDVMLENYRNLVSL

SEQ ID NO: 280 - protein, 80 aa, synthetic construct
KTRKRKRKAKESGMALLQGLLTFRDVAIEFSQEEWKCLDPAQRTLYRDVMLENYRNLVSLDTSSKCMMKMFSSTGQGNTE

SEQ ID NO: 281 - protein, 41 aa, synthetic construct
SLTFKDVFVDFTLEEWQQLDSAQKNLYRDVMLENYSHLVSV

SEQ ID NO: 282 - protein, 80 aa, synthetic construct
AMSQESLTFKDVFVDFTLEEWQQLDSAQKNLYRDVMLENYSHLVSVGHLVGKPDVIFRLGPGDESWMADGGTPVRTCAGE

SEQ ID NO: 283 - protein, 41 aa, synthetic construct
SVAFEDVAVNFTLEEWALLDPSQKNLYRDVMRETFRNLASV

SEQ ID NO: 284 - protein, 80 aa, synthetic construct
DSVAFEDVAVNFTLEEWALLDPSQKNLYRDVMRETFRNLASVGKQWEDQNIEDPFKIPRRNISHIPERLCESKEGGQGEE

SEQ ID NO: 285 - protein, 41 aa, synthetic construct
SVAFEDVAVSFTQEEWALLDPSQKNLYRDVMQETFKNLTSV

SEQ ID NO: 286 - protein, 80 aa, synthetic construct
MFQDSVAFEDVAVSFTQEEWALLDPSQKNLYRDVMQETFKNLTSVGKTWKVQNIEDEYKNPRRNLSLMREKLCESKESHH

SEQ ID NO: 287 - protein, 41 aa, synthetic construct
TVTFEDVAVKFTQEEWNLLSEAQRCLYRDVTLENLALMSSL

SEQ ID NO: 288 - protein, 80 aa, synthetic construct
AVVATLRLSAQGTVTFEDVAVKFTQEEWNLLSEAQRCLYRDVTLENLALMSSLGCWCGVEDEAAPSKQSIYIQRETQVRT

SEQ ID NO: 289 - protein, 41 aa, synthetic construct
LVTFEDVAVYFSLEEWERLEADQRGLYQEVMQENYGILVSL

SEQ ID NO: 290 - protein, 80 aa, synthetic construct
EEEEEDEDEDDLLTAGCQELVTFEDVAVYFSLEEWERLEADQRGLYQEVMQENYGILVSLGYPIPKPDLIFRLEQGEEPW

SEQ ID NO: 291 - protein, 41 aa, synthetic construct
MVTFKDVAVVFTREELGLLDLAQRKLYQDVMLENFRNLLSV

SEQ ID NO: 292 - protein, 80 aa, synthetic construct
TKFQEMVTFKDVAVVFTREELGLLDLAQRKLYQDVMLENFRNLLSVGYQPFKLDVILQLGKEDKLRMMETEIQGDGCSGH

SEQ ID NO: 293 - protein, 41 aa, synthetic construct
RLTFRDVAIEFSLAEWKCLNPSQRALYREVMLENYRNLEAV

SEQ ID NO: 294 - protein, 80 aa, synthetic construct
EEAAQKRKGKEPGMALPQGRLTFRDVAIEFSLAEWKCLNPSQRALYREVMLENYRNLEAVDISSKCMMKEVLSTGQGNTE

SEQ ID NO: 295 - protein, 41 aa, synthetic construct
TVVFEDVVVDFTLEEWALLNPAQRKLYRDVMLETFKHLASV

SEQ ID NO: 296 - protein, 80 aa, synthetic construct
DTVVFEDVVVDFTLEEWALLNPAQRKLYRDVMLETFKHLASVDNEAQLKASGSISQQDTSGEKLSLKQKIEKFTRKNIWA

SEQ ID NO: 297 - protein, 41 aa, synthetic construct
GLTFKDVAVVFTEEELGLLDPVQRNLYQDVMLENFRNLLSV

SEQ ID NO: 298 - protein, 80 aa, synthetic construct
TTFKEGLTFKDVAVVFTEEELGLLDPVQRNLYQDVMLENFRNLLSVGHHPFKHDVFLLEKEKKLDIMKTATQRKGKSADK

SEQ ID NO: 299 - protein, 41 aa, synthetic construct
LVTFDSVAVEFTQEEWTLLDPAQRNLYSDVMLENYKNLSSV

SEQ ID NO: 300 - protein, 80 aa, synthetic construct
SALQQEFWKIQTSNGIQMDLVTFDSVAVEFTQEEWTLLDPAQRNLYSDVMLENYKNLSSVGYQLFKPSLISWLEEEEELS

SEQ ID NO: 301 - protein, 41 aa, synthetic construct
CVIFEEVAVNFTPEEWALLDHAQRSLYRDVMLETCRNLASL

SEQ ID NO: 302 - protein, 80 aa, synthetic construct
DCVIFEEVAVNFTPEEWALLDHAQRSLYRDVMLETCRNLASLDCYIYVRTSGSSSQRDVFGNGISNDEEIVKFTGSDSWS

SEQ ID NO: 303 - protein, 41 aa, synthetic construct
LLTFRDVTIEFSLEEWEFLNPAQQSLYRKVMLENYRNLVSL

SEQ ID NO: 304 - protein, 80 aa, synthetic construct
ELLTFRDVTIEFSLEEWEFLNPAQQSLYRKVMLENYRNLVSLGLTVSKPELISRLEQRQEPWNVKRHETIAKPPAMSSHY

SEQ ID NO: 305 - protein, 41 aa, synthetic construct
SLTLEDVAVEFSWEEWQLLDTAQKNLYRDVMVENYNHLVSL

SEQ ID NO: 306 - protein, 80 aa, synthetic construct
IKTQESLTLEDVAVEFSWEEWQLLDTAQKNLYRDVMVENYNHLVSLGYQTSKPDVLSKLAHGQEPWTTDAKIQNKNCPGI

SEQ ID NO: 307 - protein, 41 aa, synthetic construct
AVSFADVAVYFSPEEWECLRPAQRALYRDVMRETFGHLGAL

SEQ ID NO: 308 - protein, 80 aa, synthetic construct
PAHVPGEAGPRRTRESRPGAVSFADVAVYFSPEEWECLRPAQRALYRDVMRETFGHLGALGFSVPKPAFISWVEGEVEAW

SEQ ID NO: 309 - protein, 40 aa, synthetic construct
TMTFKDVEVTFSQDEWGWLDSAQRNLYRDVMLENYRNMAS

SEQ ID NO: 310 - protein, 80 aa, synthetic construct
GCPGDQVTPTRSLTAQLQETMTFKDVEVTFSQDEWGWLDSAQRNLYRDVMLENYRNMASLVGPFTKPALISWLEAREPWG

SEQ ID NO: 311 - protein, 40 aa, synthetic construct
LVMFRDVAVDFSQEEWECLNSYQRNLYRDVILENYSNLVS

SEQ ID NO: 312 - protein, 80 aa, synthetic construct
ARDLVMFRDVAVDFSQEEWECLNSYQRNLYRDVILENYSNLVSLAGCSISKPDVITLLEQGKEPWMVVRDEKRRWTLDLE

SEQ ID NO: 313 - protein, 41 aa, synthetic construct
AVTFKDVAVVFTEEELRLLDLAQRKLYREVMLENFRNLLSV

SEQ ID NO: 314 - protein, 80 aa, synthetic construct
TTLKEAVTFKDVAVVFTEEELRLLDLAQRKLYREVMLENFRNLLSVGHQSLHRDTFHFLKEEKFWMMETATQREGNLGGK

SEQ ID NO: 315 - protein, 41 aa, synthetic construct
GMTFEDVAIYFSQEEWELLDESQRFLYCDVMLENFAHVTSL

SEQ ID NO: 316 - protein, 80 aa, synthetic construct
SPPSPRSSMAAVALRDSAQGMTFEDVAIYFSQEEWELLDESQRFLYCDVMLENFAHVTSLGYCHGMENEAIASEQSVSIQ

SEQ ID NO: 317 - protein, 41 aa, synthetic construct
RLTFMDVAIEFSQEEWKSLDPGQRALYRDVMLENYRNLVFL

SEQ ID NO: 318 - protein, 80 aa, synthetic construct
DEEAQKRKAKESGMALPQGRLTFMDVAIEFSQEEWKSLDPGQRALYRDVMLENYRNLVFLGICLPDLSIISMLKQRREPL

SEQ ID NO: 319 - protein, 41 aa, synthetic construct
PLKFMDVAIEFSQEEWKCLDPAQRTLYRDVMLENYRNLVSL

SEQ ID NO: 320 - protein, 80 aa, synthetic construct
ALTQGPLKFMDVAIEFSQEEWKCLDPAQRTLYRDVMLENYRNLVSLGICLPDLSVTSMLEQKRDPWTLQSEEKIANDPDG

SEQ ID NO: 321 - protein, 41 aa, synthetic construct
AVTFKDVAVVFTEEELGLLDVSQRKLYRDVMLENFRNLLSV

SEQ ID NO: 322 - protein, 80 aa, synthetic construct
TMFKEAVTFKDVAVVFTEEELGLLDVSQRKLYRDVMLENFRNLLSVGHQLSHRDTFHFQREEKFWIMETATQREGNSGGK

SEQ ID NO: 323 - protein, 41 aa, synthetic construct
TVAFEDVAVNFSQEEWSLLSEVQRCLYHDVMLENWVLISSL

SEQ ID NO: 324 - protein, 80 aa, synthetic construct
QGTVAFEDVAVNFSQEEWSLLSEVQRCLYHDVMLENWVLISSLGCWCGSEDEEAPSKKSISIQRVSQVSTPGAGVSPKKA

SEQ ID NO: 325 - protein, 50 aa, synthetic construct
FEVEKILDMKTEGGKVLYKVRWKGYTSDDDTWEPEIHLEDCKEVLLEFRK

SEQ ID NO: 326 - protein, 80 aa, synthetic construct
AEAFGDSEEDGEDVFEVEKILDMKTEGGKVLYKVRWKGYTSDDDTWEPEIHLEDCKEVLLEFRKKIAENKAKAVRKDIQR

SEQ ID NO: 327 - protein, 41 aa, synthetic construct
SISLEDVAVNFTLEEWALLDPGQRNIYRDVMRATFKNLACI

SEQ ID NO: 328 - protein, 80 aa, synthetic construct
VLQMQNSEHHGQSIKTQTDSISLEDVAVNFTLEEWALLDPGQRNIYRDVMRATFKNLACIGEKWKDQDIEDEHKNQGRNL

SEQ ID NO: 329 - protein, 41 aa, synthetic construct
SVTFDDVAVTFTQEEWGQLDLAQRTLYQEVMLENCGLLVSL

SEQ ID NO: 330 - protein, 80 aa, synthetic construct
AMALTDPAQVSVTFDDVAVTFTQEEWGQLDLAQRTLYQEVMLENCGLLVSLGCPVPRPELIYHLEHGQEPWTRKEDLSQG

SEQ ID NO: 331 - protein, 41 aa, synthetic construct
SVTFRDVAIDFSQEEWECLQPDQRTLYRDVMLENYSHLISL

SEQ ID NO: 332 - protein, 80 aa, synthetic construct
VHGSVTFRDVAIDFSQEEWECLQPDQRTLYRDVMLENYSHLISLGSSISKPDVITLLEQEKEPWIVVSKETSRWYPDLES

SEQ ID NO: 333 - protein, 41 aa, synthetic construct
PVACEDVAVNFTQEEWALLDISQRKLYREVMLETFRNLTSI

SEQ ID NO: 334 - protein, 80 aa, synthetic construct
DPVACEDVAVNFTQEEWALLDISQRKLYREVMLETFRNLTSIGKKWKDQNIEYEYQNPRRNFRSLIEGNVNEIKEDSHCG

SEQ ID NO: 335 - protein, 40 aa, synthetic construct
RVTFKDVAVVFTKEELALLDKAQINLYQDVMLENFRNLML

SEQ ID NO: 336 - protein, 80 aa, synthetic construct
IKFQERVTFKDVAVVFTKEELALLDKAQINLYQDVMLENFRNLMLVRDGIKNNILNLQAKGLSYLSQEVLHCWQIWKQRI

SEQ ID NO: 337 - protein, 41 aa, synthetic construct
PLTFRDVAIEFSLKEWQCLDTAQRNLYRNVMLENYRNLVFL

SEQ ID NO: 338 - protein, 80 aa, synthetic construct
GPLTFRDVAIEFSLKEWQCLDTAQRNLYRNVMLENYRNLVFLGITVSKPDLITCLEQGKEAWSMKRHEIMVAKPTVMCSH

SEQ ID NO: 339 - protein, 41 aa, synthetic construct
AVTFKDVAVVFTEEELGLLDLAQRKLYRDVMLENFRNLLSV

SEQ ID NO: 340 - protein, 80 aa, synthetic construct
TMSKEAVTFKDVAVVFTEEELGLLDLAQRKLYRDVMLENFRNLLSVGHQPFHRDTFHFLREEKFWMMDIATQREGNSGGK

SEQ ID NO: 341 - protein, 41 aa, synthetic construct
PLEFRDVAIEFSLEEWHCLDTAQQNLYRDVMLENYRHLVFL

SEQ ID NO: 342 - protein, 80 aa, synthetic construct
GPLEFRDVAIEFSLEEWHCLDTAQQNLYRDVMLENYRHLVFLGIVVTKPDLITCLEQGKKPFTVKRHEMIAKSPVMCFHF

SEQ ID NO: 343 - protein, 41 aa, synthetic construct
LVTFEDVAVEFTQEEWALLDPAQRTLYRDVMLENCRNLASL

SEQ ID NO: 344 - protein, 80 aa, synthetic construct
GHTEGGELVNELLKSWLKGLVTFEDVAVEFTQEEWALLDPAQRTLYRDVMLENCRNLASLGNQVDKPRLISQLEQEDKVM

SEQ ID NO: 345 - protein, 41 aa, synthetic construct
TVTFDDVALYFSEQEWEILEKWQKQMYKQEMKTNYETLDSL

SEQ ID NO: 346 - protein, 80 aa, synthetic construct
AEPASVTVTFDDVALYFSEQEWEILEKWQKQMYKQEMKTNYETLDSLGYAFSKPDLITWMEQGRMLLISEQGCLDKTRRT

SEQ ID NO: 347 - protein, 41 aa, synthetic construct
PLSFKDVAVVFTEEELELLDSTQRQLYQDVMQENFRNLLSV

SEQ ID NO: 348 - protein, 80 aa, synthetic construct
HSQASAISQDREEKIMSQEPLSFKDVAVVFTEEELELLDSTQRQLYQDVMQENFRNLLSVGERNPLGDKNGKDTEYIQDE

SEQ ID NO: 349 - protein, 41 aa, synthetic construct
PVTFKDVAVDFTQEEWGQLDLVQRTLYRDVMLETYGHLLSV

SEQ ID NO: 350 - protein, 80 aa, synthetic construct
GSLEEGRRATGLPAAQVQEPVTFKDVAVDFTQEEWGQLDLVQRTLYRDVMLETYGHLLSVGNQIAKPEVISLLEQGEEPW

SEQ ID NO: 351 - protein, 41 aa, synthetic construct
AVTFKDVAVVFTEEELGLLDPAQRKLYRDVMLENFRNLLSV

SEQ ID NO: 352 - protein, 80 aa, synthetic construct
TTFKEAVTFKDVAVVFTEEELGLLDPAQRKLYRDVMLENFRNLLSVGHQPFHQDTCHFLREEKFWMMGTATQREGNSGGK

SEQ ID NO: 353 - protein, 41 aa, synthetic construct
AVTFKDVAVIFTEEELGLLDPAQRKLYRDVMLENFRNLLSV

SEQ ID NO: 354 - protein, 80 aa, synthetic construct
AKLYEAVTFKDVAVIFTEEELGLLDPAQRKLYRDVMLENFRNLLSVGGKIQTEMETVPEAGTHEEFSCKQIWEQIASDLT

SEQ ID NO: 355 - protein, 41 aa, synthetic construct
SVAFEDVAVNFTQEEWALLGPSQKSLYRDVMWETIRNLDCI

SEQ ID NO: 356 - protein, 80 aa, synthetic construct
RSDLFLPDSQTNEERKQYDSVAFEDVAVNFTQEEWALLGPSQKSLYRDVMWETIRNLDCIGMKWEDTNIEDQHRNPRRSL

SEQ ID NO: 357 - protein, 41 aa, synthetic construct
LLTFRDVAIEFSPEEWQCLDTAQQNLYRNVMLENYRNLAFL

SEQ ID NO: 358 - protein, 80 aa, synthetic construct
PGTPGSLEMGLLTFRDVAIEFSPEEWQCLDTAQQNLYRNVMLENYRNLAFLGIALSKPDLITYLEQGKEPWNMKQHEMVD

SEQ ID NO: 359 - protein, 41 aa, synthetic construct
QVTFEDVVVGFSQEEWGQLKPAQRTLYRDVMLDTFRLLVSV

SEQ ID NO: 360 - protein, 80 aa, synthetic construct
TPGVRVSTDPEQVTFEDVVVGFSQEEWGQLKPAQRTLYRDVMLDTFRLLVSVGHWLPKPNVISLLEQEAELWAVESRLPQ

SEQ ID NO: 361 - protein, 41 aa, synthetic construct
AVTFDDVAVDFTQEEWTLLDPSQRDLYRDVMLENYENLASV

SEQ ID NO: 362 - protein, 80 aa, synthetic construct
EQTQAAGMVAGWLINCYQDAVTFDDVAVDFTQEEWTLLDPSQRDLYRDVMLENYENLASVEWRLKTKGPALRQDRSWFRA

SEQ ID NO: 363 - protein, 32 aa, synthetic construct
RPRLKNVDRSTAQQLAVTVGNVTVIITDFKEK

SEQ ID NO: 364 - protein, 80 aa, synthetic construct
PSEANSIQSANATTKTSETNHTSRPRLKNVDRSTAQQLAVTVGNVTVIITDFKEKTRSSSTSSSTVTSSAGSEQQNQSSS

SEQ ID NO: 365 - protein, 41 aa, synthetic construct
QLSFSDVAIEFSQEEWKCLDPGQKALYRDVMLENYRNLVSL

SEQ ID NO: 366 - protein, 80 aa, synthetic construct
ALTQGQLSFSDVAIEFSQEEWKCLDPGQKALYRDVMLENYRNLVSLGEDNVRPEACICSGICLPDLSVTSMLEQKRDPWT

SEQ ID NO: 367 - protein, 41 aa, synthetic construct
SVTFEDVAVNFSLEEWSLLNEAQRCLYRDVMLETLTLISSL

SEQ ID NO: 368 - protein, 80 aa, synthetic construct
AAAAALRAPAQSSVTFEDVAVNFSLEEWSLLNEAQRCLYRDVMLETLTLISSLGCWHGGEDEAAPSKQSTCIHIYKDQGG

SEQ ID NO: 369 - protein, 41 aa, synthetic construct
SVSFNDVTVDFTQEEWQHLDHAQKTLYMDVMLENYCHLISV

SEQ ID NO: 370 - protein, 80 aa, synthetic construct
AQGSVSFNDVTVDFTQEEWQHLDHAQKTLYMDVMLENYCHLISVGCHMTKPDVILKLERGEEPWTSFAGHTCLEENWKAE

SEQ ID NO: 371 - protein, 41 aa, synthetic construct
PVAFKDVAVNFTQEEWALLDISQRKLYREVMLETFRNLTSL

SEQ ID NO: 372 - protein, 80 aa, synthetic construct
DPVAFKDVAVNFTQEEWALLDISQRKLYREVMLETFRNLTSLGKRWKDQNIEYEHQNPRRNFRSLIEEKVNEIKDDSHCG

SEQ ID NO: 373 - protein, 41 aa, synthetic construct
LVTFGDVAVNFSQEEWEWLNPAQRNLYRKVMLENYRSLVSL

SEQ ID NO: 374 - protein, 80 aa, synthetic construct
SKDLVTFGDVAVNFSQEEWEWLNPAQRNLYRKVMLENYRSLVSLGVSVSKPDVISLLEQGKEPWMVKKEGTRGPCPDWEY

SEQ ID NO: 375 - protein, 41 aa, synthetic construct
SVAFEDVAINFTCEEWALLGPSQKSLYRDVMQETIRNLDCI

SEQ ID NO: 376 - protein, 80 aa, synthetic construct
DSVAFEDVAINFTCEEWALLGPSQKSLYRDVMQETIRNLDCIGMIWQNHDIEEDQYKDLRRNLRCHMVERACEIKDNSQC

SEQ ID NO: 377 - protein, 41 aa, synthetic construct
PLTFMDVAIEFCLEEWQCLDIAQQNLYRNVMLENYRNLVFL

SEQ ID NO: 378 - protein, 80 aa, synthetic construct
GPLTFMDVAIEFCLEEWQCLDIAQQNLYRNVMLENYRNLVFLGIAVSKPDLITCLEQEKEPWEPMRRHEMVAKPPVMCSH

SEQ ID NO: 379 - protein, 52 aa, synthetic construct
EPEKIIGATDSCGDLMFLMKWKDTDEADLVLAKEANVKCPQIVIAFYEERLT

SEQ ID NO: 380 - protein, 80 aa, synthetic construct
QSNDIARGFERGLEPEKIIGATDSCGDLMFLMKWKDTDEADLVLAKEANVKCPQIVIAFYEERLTWHAYPEDAENKEKET

SEQ ID NO: 381 - protein, 40 aa, synthetic construct
PVTFEDVAVLFTEAEWKRLSLEQRNLYKEVMLENLRNLVS

SEQ ID NO: 382 - protein, 80 aa, synthetic construct
ALPAKDSAWPWEEKPRYLGPVTFEDVAVLFTEAEWKRLSLEQRNLYKEVMLENLRNLVSLAESKPEVHTCPSCPLAFGSQ

SEQ ID NO: 383 - protein, 41 aa, synthetic construct
LVTFKDVFVDFTREEWKLLDTAQQIVYRNVMLENYKNLVSL

SEQ ID NO: 384 - protein, 80 aa, synthetic construct
DAKSLTAWSRTLVTFKDVFVDFTREEWKLLDTAQQIVYRNVMLENYKNLVSLGYQLTKPDVILRLEKGEEPWLVEREIHQ

SEQ ID NO: 385 - protein, 40 aa, synthetic construct
AVAFEDVAVNFTQEEWALLGPSQKNLYRYVMQETIRNLDC

SEQ ID NO: 386 - protein, 80 aa, synthetic construct
DAVAFEDVAVNFTQEEWALLGPSQKNLYRYVMQETIRNLDCIRMIWEEQNTEDQYKNPRRNLRCHMVERFSESKDSSQCG

SEQ ID NO: 387 - protein, 41 aa, synthetic construct
SVTFDDVAVDFTPEEWALLDTTEKYLYRDVMLENYMNLASV

SEQ ID NO: 388 - protein, 80 aa, synthetic construct
EKTKVERMVEDYLASGYQDSVTFDDVAVDFTPEEWALLDTTEKYLYRDVMLENYMNLASVEWEIQPRTKRSSLQQGFLKN

SEQ ID NO: 389 - protein, 41 aa, synthetic construct
SVAFEDVDVNFTQEEWALLDPSQKNLYRDVMWETMRNLASI

SEQ ID NO: 390 - protein, 80 aa, synthetic construct
DSVAFEDVDVNFTQEEWALLDPSQKNLYRDVMWETMRNLASIGKKWKDQNIKDHYKHRGRNLRSHMLERLYQTKDGSQRG

SEQ ID NO: 391 - protein, 41 aa, synthetic construct
PVTFEDVAVDFTQEEWQQLNPAQKTLHRDVMLETYNHLVSV

SEQ ID NO: 392 - protein, 80 aa, synthetic construct
IESQEPVTFEDVAVDFTQEEWQQLNPAQKTLHRDVMLETYNHLVSVGCSGIKPDVIFKLEHGKDPWIIESELSRWIYPDR

SEQ ID NO: 393 - protein, 41 aa, synthetic construct
LVTFRDVALDFSQEEWEWLKPSQKDLYRDVMLENYRNLVWL

SEQ ID NO: 394 - protein, 80 aa, synthetic construct
AVGLCKAMSQGLVTFRDVALDFSQEEWEWLKPSQKDLYRDVMLENYRNLVWLGLSISKPNMISLLEQGKEPWMVERKMSQ

SEQ ID NO: 395 - protein, 40 aa, synthetic construct
PVTFADVAVVFTPEEWVFLDSTQRSLYRDVMLENYRNLAS

SEQ ID NO: 396 - protein, 80 aa, synthetic construct
DKVEEEAMAPGLPTACSQEPVTFADVAVVFTPEEWVFLDSTQRSLYRDVMLENYRNLASVADQLCKPNALSYLEERGEQW

SEQ ID NO: 397 - protein, 40 aa, synthetic construct
MTFEDVAVYFSQEEWGLLDTAQRALYRHVMLENFTLVTSL

SEQ ID NO: 398 - protein, 80 aa, synthetic construct
TFEDVAVYFSQEEWGLLDTAQRALYRHVMLENFTLVTSLGLSTSRPRVVIQLERGEEPWVPSGKDMTLARNTYGRLNSGS

SEQ ID NO: 399 - protein, 41 aa, synthetic construct
PLTFEDVAIYFSEQEWQDLEAWQKELYKHVMRSNYETLVSL

SEQ ID NO: 400 - protein, 80 aa, synthetic construct
AEPPRLPLTFEDVAIYFSEQEWQDLEAWQKELYKHVMRSNYETLVSLDDGLPKPELISWIEHGGEPFRKWRESQKSGNII

SEQ ID NO: 401 - protein, 41 aa, synthetic construct
SVVFEDVAVNFTQEEWALLGPSQKKLYRDVMQETFVNLASI

SEQ ID NO: 402 - protein, 80 aa, synthetic construct
DSVVFEDVAVNFTQEEWALLGPSQKKLYRDVMQETFVNLASIGENWEEKNIEDHKNQGRKLRSHMVERLCERKEGSQFGE

SEQ ID NO: 403 - protein, 41 aa, synthetic construct
CVTFEDVTIYFSQEEWVLLDEAQRLLYCDVMLENFALIASL

SEQ ID NO: 404 - protein, 80 aa, synthetic construct
AAAALRDPAQGCVTFEDVTIYFSQEEWVLLDEAQRLLYCDVMLENFALIASLGLISFRSHIVSQLEMGKEPWVPDSVDMT

SEQ ID NO: 405 - protein, 41 aa, synthetic construct
LVSFEDVVVTFTGEEWGHLDLAQRTLYQEVMLETCRLLVSL

SEQ ID NO: 406 - protein, 80 aa, synthetic construct
AAPALALVSFEDVVVTFTGEEWGHLDLAQRTLYQEVMLETCRLLVSLGHPVPKPELIYLLEHGQELWTVKRGLSQSTCAG

SEQ ID NO: 407 - protein, 41 aa, synthetic construct
SLTLEDVAVEFTWEEWQLLGPAQKDLYRDVMLENYSNLVSV

SEQ ID NO: 408 - protein, 80 aa, synthetic construct
IKSQESLTLEDVAVEFTWEEWQLLGPAQKDLYRDVMLENYSNLVSVGYQASKPDALFKLEQGEPWTVENEIHSQICPEIK

SEQ ID NO: 409 - protein, 41 aa, synthetic construct
LLTFKDVSVDFTQEEWGQLAPAHRNLYREVMLENYGNLVSV

SEQ ID NO: 410 - protein, 80 aa, synthetic construct
GESLESRVTLGSLTAESQELLTFKDVSVDFTQEEWGQLAPAHRNLYREVMLENYGNLVSVGCQLSKPGVISQLEKGEEPW

SEQ ID NO: 411 - protein, 41 aa, synthetic construct
SVALEDVAVNFTREEWALLGPCQKNLYKDVMQETIRNLDCV

SEQ ID NO: 412 - protein, 80 aa, synthetic construct
ASVALEDVAVNFTREEWALLGPCQKNLYKDVMQETIRNLDCVGMKWKDQNIEDQYRYPRKNLRCRMLERFVESKDGTQCG

SEQ ID NO: 413 - protein, 41 aa, synthetic construct
TVTFKDVAIDFTQEEWKRLDPAQRKLYRNVMLENYNNLITV

SEQ ID NO: 414 - protein, 80 aa, synthetic construct
TESQGTVTFKDVAIDFTQEEWKRLDPAQRKLYRNVMLENYNNLITVGYPFTKPDVIFKLEQEEEPWVMEEEVLRRHWQGE

SEQ ID NO: 415 - protein, 41 aa, synthetic construct
SVASEDVAVNFTLEEWALLDPSQKKLYRDVMRETFRNLACV

SEQ ID NO: 416 - protein, 80 aa, synthetic construct
DSVASEDVAVNFTLEEWALLDPSQKKLYRDVMRETFRNLACVGKKWEDQSIEDWYKNQGRILRNHMEEGLSESKEYDQCG

SEQ ID NO: 417 - protein, 41 aa, synthetic construct
SLAFRDVSIDLSQEEWECLDAVQRDLYKDVMLENYSNLVSL

SEQ ID NO: 418 - protein, 80 aa, synthetic construct
EETQGELTSSCGSKTMANVSLAFRDVSIDLSQEEWECLDAVQRDLYKDVMLENYSNLVSLGYTIPKPDVITLLEQEKEPW

SEQ ID NO: 419 - protein, 41 aa, synthetic construct
PVSFEDVSVYFTKTEWKLLDLRQKVLYKRVMLENYSHLVSL

SEQ ID NO: 420 - protein, 80 aa, synthetic construct
AAILLTTRPKVPVSFEDVSVYFTKTEWKLLDLRQKVLYKRVMLENYSHLVSLGFSFSKPHLISQLERGEGPWVADIPRTW

SEQ ID NO: 421 - protein, 32 aa, synthetic construct
RPRLKNVDRSSAQHLEVTVGDLTVIITDFKEK

SEQ ID NO: 422 - protein, 80 aa, synthetic construct
KDKVEKEKSEKETTSKKNSHKKTRPRLKNVDRSSAQHLEVTVGDLTVIITDFKEKTKSPPASSAASADQHSQSGSSSDNT

SEQ ID NO: 423 - protein, 41 aa, synthetic construct
PLTFTDVAIKFSLEEWQFLDTAQQNLYRDVMLENYRNLVFL

SEQ ID NO: 424 - protein, 80 aa, synthetic construct
GPLTFTDVAIKFSLEEWQFLDTAQQNLYRDVMLENYRNLVFLGVGVSKPDLITCLEQGKEPWNMKRHKMVAKPPVVCSHF

SEQ ID NO: 425 - protein, 41 aa, synthetic construct
EVTFEDVAVYLSREEWGRLGPAQRGLYRDVMLETYGNLVSL

SEQ ID NO: 426 - protein, 80 aa, synthetic construct
RKPNPQAMAALFLSAPPQAEVTFEDVAVYLSREEWGRLGPAQRGLYRDVMLETYGNLVSLGVGPAGPKPGVISQLERGDE

SEQ ID NO: 427 - protein, 41 aa, synthetic construct
PVAFKDVAVNFTQEEWALLDISQKNLYREVMLETFWNLTSI

SEQ ID NO: 428 - protein, 80 aa, synthetic construct
LSLSPILLYTCEMFQDPVAFKDVAVNFTQEEWALLDISQKNLYREVMLETFWNLTSIGKKWKDQNIEYEYQNPRRNFRSV

SEQ ID NO: 429 - protein, 40 aa, synthetic construct
PVTFEDVAVNFTQEEWDCLDASQRVLYQDVMSETFKNLTS

SEQ ID NO: 430 - protein, 80 aa, synthetic construct
AAGEPRSLLFFQKPVTFEDVAVNFTQEEWDCLDASQRVLYQDVMSETFKNLTSVARIFLHKPELITKLEQEEEQWRETRV

SEQ ID NO: 431 - protein, 41 aa, synthetic construct
MVTFEDVAVHFTKTEWTGLSPAQRALYRSVMLENFGNLTAL

SEQ ID NO: 432 - protein, 80 aa, synthetic construct
AAMPLKAQYQEMVTFEDVAVHFTKTEWTGLSPAQRALYRSVMLENFGNLTALGYPVPKPALISLLERGDMAWGLEAQDDP

SEQ ID NO: 433 - protein, 41 aa, synthetic construct
PLTFSDVAIDFSQEEWEYLNSDQRDLYRDVMLENYTNLVSL

SEQ ID NO: 434 - protein, 80 aa, synthetic construct
ARVPLTFSDVAIDFSQEEWEYLNSDQRDLYRDVMLENYTNLVSLDFNFTTESNKLSSEKRNYEVNAYHQETWKRNKTFNL

SEQ ID NO: 435 - protein, 41 aa, synthetic construct
PVTFEDVTLGFTPEEWGLLDLKQKSLYREVMLENYRNLVSV

SEQ ID NO: 436 - protein, 80 aa, synthetic construct
ASRLPTAWSCEPVTFEDVTLGFTPEEWGLLDLKQKSLYREVMLENYRNLVSVEHQLSKPDVVSQLEEAEDFWPVERGIPQ

SEQ ID NO: 437 - protein, 52 aa, synthetic construct
DPERIIGATDSSGELMFLMKWKDSDEADLVLAKEANMKCPQIVIAFYEERLT

SEQ ID NO: 438 - protein, 80 aa, synthetic construct
SKKKRDAADKPRGFARGLDPERIIGATDSSGELMFLMKWKDSDEADLVLAKEANMKCPQIVIAFYEERLTWHSCPEDEAQ

SEQ ID NO: 439 - protein, 41 aa, synthetic construct
SVTFEDVAIAFSQQEWESLDSSQRGLYRDVMLENYRNLVSM

SEQ ID NO: 440 - protein, 80 aa, synthetic construct
AHKYVGLQYHGSVTFEDVAIAFSQQEWESLDSSQRGLYRDVMLENYRNLVSMGHSRSKPHVIALLEQWKEPEVTVRKDGR

SEQ ID NO: 441 - protein, 41 aa, synthetic construct
KLTFEDVAVLLSQDEWDRLCPAQRGLYRNVMMETYGNVVSL

SEQ ID NO: 442 - protein, 80 aa, synthetic construct
AAARLLPVPAGPQPLSFQAKLTFEDVAVLLSQDEWDRLCPAQRGLYRNVMMETYGNVVSLGLPGSKPDIISQLERGEDPW

SEQ ID NO: 443 - protein, 41 aa, synthetic construct
LVTFRDVAVDFSQEEWDCLDSSQRHLYSNVMLENYRILVSL

SEQ ID NO: 444 - protein, 80 aa, synthetic construct
AVGLLKAMYQELVTFRDVAVDFSQEEWDCLDSSQRHLYSNVMLENYRILVSLGLCFSKPSVILLLEQGKAPWMVKRELTK

SEQ ID NO: 445 - protein, 41 aa, synthetic construct
LLTFRDVAIEFSLEEWQCLDTAQRNLYKNVILENYRNLVFL

SEQ ID NO: 446 - protein, 80 aa, synthetic construct
GLLTFRDVAIEFSLEEWQCLDTAQRNLYKNVILENYRNLVFLGIAVSKQDLITCLEQEKEPLTVKRHEMVNEPPVMCSHF

SEQ ID NO: 447 - protein, 41 aa, synthetic construct
LLAFRDVALEFSPEEWECLDPAQRSLYRDVMLENYRNLISL

SEQ ID NO: 448 - protein, 80 aa, synthetic construct
GLLAFRDVALEFSPEEWECLDPAQRSLYRDVMLENYRNLISLGEDSFNMQFLFHSLAMSKPELIICLEARKEPWNVNTEK

SEQ ID NO: 449 - protein, 41 aa, synthetic construct
RVVFEDVAIYFSQEEWGHLDEAQRLLYRDVMLENLALLSSL

SEQ ID NO: 450 - protein, 80 aa, synthetic construct
NLTEGRVVFEDVAIYFSQEEWGHLDEAQRLLYRDVMLENLALLSSLGSWHGAEDEEAPSQQGFSVGVSEVTASKPCLSSQ

SEQ ID NO: 451 - protein, 41 aa, synthetic construct
AVTFKDVAVVFSREELRLLDLTQRKLYRDVMVENFKNLVAV

SEQ ID NO: 452 - protein, 80 aa, synthetic construct
SQNYDLPQKKQEKMTKFQEAVTFKDVAVVFSREELRLLDLTQRKLYRDVMVENFKNLVAVGHLPFQPDMVSQLEAEEKLW

SEQ ID NO: 453 - protein, 41 aa, synthetic construct
MVTFEDVAVYFSQEEWELLDAAQRHLYHSVMLENLELVTSL

SEQ ID NO: 454 - protein, 80 aa, synthetic construct
GPAQHTSWPCGSAVPTLKSMVTFEDVAVYFSQEEWELLDAAQRHLYHSVMLENLELVTSLGSWHGVEGEGAHPKQNVSVE

SEQ ID NO: 455 - protein, 41 aa, synthetic construct
PLTFRDVVIEFSQEEWQCLDTAQQDLYRKVMLENFRNLVFL

SEQ ID NO: 456 - protein, 80 aa, synthetic construct
SGYPGAERNLLEYSYFEKGPLTFRDVVIEFSQEEWQCLDTAQQDLYRKVMLENFRNLVFLGIDVSKPDLITCLEQGKDPW

SEQ ID NO: 457 - protein, 41 aa, synthetic construct
LVMFRDVAIDFSQEEWECLDSAQRDLYRDVMLENYSNLVSL

SEQ ID NO: 458 - protein, 80 aa, synthetic construct
ARKLVMFRDVAIDFSQEEWECLDSAQRDLYRDVMLENYSNLVSLDLPSRCASKDLSPEKNTYETELSQWEMSDRLENCDL

SEQ ID NO: 459 - protein, 40 aa, synthetic construct
MTFEDVAVEFSQWEWGQLNPAQKDLYREVMLENFRNLAIL

SEQ ID NO: 460 - protein, 80 aa, synthetic construct
TFEDVAVEFSQWEWGQLNPAQKDLYREVMLENFRNLAILGLLVSKPYVICQLEEGGEPFMVEREISTGAHSDWKRRSKSK

SEQ ID NO: 461 - protein, 41 aa, synthetic construct
PLQFRDVAIEFSLEEWHCLDTAQRNLYRNVMLENYSNLVFL

SEQ ID NO: 462 - protein, 80 aa, synthetic construct
GPLQFRDVAIEFSLEEWHCLDTAQRNLYRNVMLENYSNLVFLGITVSKPDLITCLEQGRKPLTMKRNEMIAKPSVMCSHF

SEQ ID NO: 463 - protein, 41 aa, synthetic construct
RVTFKDIAVIFTKEELAVLDKAQINLYQDVMLENFRNFISV

SEQ ID NO: 464 - protein, 80 aa, synthetic construct
IKFQERVTFKDIAVIFTKEELAVLDKAQINLYQDVMLENFRNFISVDGIKNNILNLQGKGLIYLSQEELHCWKIWKQRIR

SEQ ID NO: 465 - protein, 41 aa, synthetic construct
SVTFEDVAVDFTQEEWTLLDQTQRNLYRDVMLENYKNLVAV

SEQ ID NO: 466 - protein, 80 aa, synthetic construct
VAGWLTNYSQDSVTFEDVAVDFTQEEWTLLDQTQRNLYRDVMLENYKNLVAVDWESHINTKWSAPQQNFLQGKTSSVVEM

SEQ ID NO: 467 - protein, 41 aa, synthetic construct
SVTFEDVAVTFTQEEWGQLDVTQRALYVEVMLETCGLLVAL

SEQ ID NO: 468 - protein, 80 aa, synthetic construct
AAAWMAPAQESVTFEDVAVTFTQEEWGQLDVTQRALYVEVMLETCGLLVALGDSTKPETVEPIPSHLALPEEVSLQEQLA

SEQ ID NO: 469 - protein, 41 aa, synthetic construct
PLSFMDVFVDFTWEEWQLLDPAQKCLYRSVMLENYSNLVSL

SEQ ID NO: 470 - protein, 80 aa, synthetic construct
VLEWLFISQEQPKITKSWGPLSFMDVFVDFTWEEWQLLDPAQKCLYRSVMLENYSNLVSLGYQHTKPDIIFKLEQGEELC

SEQ ID NO: 471 - protein, 41 aa, synthetic construct
CVTFEDVFVYFSREEWELLEEAQRFLYRDVMLENFALVATL

SEQ ID NO: 472 - protein, 80 aa, synthetic construct
AAAVLMDRVQSCVTFEDVFVYFSREEWELLEEAQRFLYRDVMLENFALVATLGFWCEAEHEAPSEQSVSVEGVSQVRTAE

SEQ ID NO: 473 - protein, 41 aa, synthetic construct
LVTFEDVLVDFSPEELSSLSAAQRNLYREVMLENYRNLVSL

SEQ ID NO: 474 - protein, 80 aa, synthetic construct
AGSQFPDFKHLGTFLVFEELVTFEDVLVDFSPEELSSLSAAQRNLYREVMLENYRNLVSLGHQFSKPDIISRLEEEESYA

SEQ ID NO: 475 - protein, 41 aa, synthetic construct
QISFEDVAVDFTLEEWQLLNPTQKNLYRDVMLENYSNLVFL

SEQ ID NO: 476 - protein, 80 aa, synthetic construct
IQSQISFEDVAVDFTLEEWQLLNPTQKNLYRDVMLENYSNLVFLEVWLDNPKMWLRDNQDNLKSMERGHKYDVFGKIFNS

SEQ ID NO: 477 - protein, 41 aa, synthetic construct
LVAFEDVAVNFTQEEWSLLDPSQKNLYREVMQETLRNLASI

SEQ ID NO: 478 - protein, 80 aa, synthetic construct
DLVAFEDVAVNFTQEEWSLLDPSQKNLYREVMQETLRNLASIGEKWKDQNIEDQYKNPRNNLRSLLGERVDENTEENHCG

SEQ ID NO: 479 - protein, 70 aa, synthetic construct
IKLRVIGQDSSEIHFKVKMTTPLKKLKKSYCQRQGVPVNSLRFLFEGQRIADNHTPEELGMEEEDVIEVY

SEQ ID NO: 480 - protein, 80 aa, synthetic construct
KDEDIKLRVIGQDSSEIHFKVKMTTPLKKLKKSYCQRQGVPVNSLRFLFEGQRIADNHTPEELGMEEEDVIEVYQEQIGG

SEQ ID NO: 481 - protein, 41 aa, synthetic construct
SVAFEDVAVNFTQEEWALLSPSQKNLYRDVTLETFRNLASV

SEQ ID NO: 482 - protein, 80 aa, synthetic construct
DSVAFEDVAVNFTQEEWALLSPSQKNLYRDVTLETFRNLASVGIQWKDQDIENLYQNLGIKLRSLVERLCGRKEGNEHRE

SEQ ID NO: 483 - protein, 41 aa, synthetic construct
PVTFDDVAVYFSELEWGKLEDWQKELYKHVMRGNYETLVSL

SEQ ID NO: 484 - protein, 80 aa, synthetic construct
RNFWILRLPPGSKGEAPKVPVTFDDVAVYFSELEWGKLEDWQKELYKHVMRGNYETLVSLDYAISKPDILTRIERGEEPC

SEQ ID NO: 485 - protein, 41 aa, synthetic construct
TVTFEDVAVNFSQEEWCLLSEAQRCLYRDVMLENLALISSL

SEQ ID NO: 486 - protein, 80 aa, synthetic construct
AAAAPRRPTQQGTVTFEDVAVNFSQEEWCLLSEAQRCLYRDVMLENLALISSLGCWCGSKDEEAPCKQRISVQRESQSRT

SEQ ID NO: 487 - protein, 41 aa, synthetic construct
LVTFEDVAVDFTQEEWQYLNPPQRTLYRDVMLETYSNLVFV

SEQ ID NO: 488 - protein, 80 aa, synthetic construct
SGEDSGSFYSWQKAKREQGLVTFEDVAVDFTQEEWQYLNPPQRTLYRDVMLETYSNLVFVGQQVTKPNLILKLEVEECPA

SEQ ID NO: 489 - protein, 41 aa, synthetic construct
SVAFEDVAVNFTQEEWALLGPSQKSLYRNVMQETIRNLDCI

SEQ ID NO: 490 - protein, 80 aa, synthetic construct
DSVAFEDVAVNFTQEEWALLGPSQKSLYRNVMQETIRNLDCIEMKWEDQNIGDQCQNAKRNLRSHTCEIKDDSQCGETFG

SEQ ID NO: 491 - protein, 41 aa, synthetic construct
SVTFQEVAVDFSQEEWALLDPAQKNLYKDVMLENFRNLASV

SEQ ID NO: 492 - protein, 80 aa, synthetic construct
AAGWLTTWSQNSVTFQEVAVDFSQEEWALLDPAQKNLYKDVMLENFRNLASVGYQLCRHSLISKVDQEQLKTDERGILQG

SEQ ID NO: 493 - protein, 41 aa, synthetic construct
LVMFEEVSVCFTSEEWACLGPIQRALYWDVMLENYGNVTSL

SEQ ID NO: 494 - protein, 80 aa, synthetic construct
ENPRNQLMALMLLTAQPQELVMFEEVSVCFTSEEWACLGPIQRALYWDVMLENYGNVTSLEWETMTENEEVTSKPSSSQR

SEQ ID NO: 495 - protein, 41 aa, synthetic construct
LVSFEEVAVHFTWEEWQDLDDAQRTLYRDVMLETYSSLVSL

SEQ ID NO: 496 - protein, 80 aa, synthetic construct
LETYNSLVSLQELVSFEEVAVHFTWEEWQDLDDAQRTLYRDVMLETYSSLVSLGHCITKPEMIFKLEQGAEPWIVEETPN

SEQ ID NO: 497 - protein, 41 aa, synthetic construct
SVAFEDVAVNFTQEEWALLDSSQKNLYREVMQETCRNLASV

SEQ ID NO: 498 - protein, 80 aa, synthetic construct
RHFRRPEPCREPLASPIQDSVAFEDVAVNFTQEEWALLDSSQKNLYREVMQETCRNLASVGSQWKDQNIEDHFEKPGKDI

SEQ ID NO: 499 - protein, 41 aa, synthetic construct
IVTFEDVAVYFSWKEWGLLDEAQKCLYHDVMLENLTLTTSL

SEQ ID NO: 500 - protein, 80 aa, synthetic construct
AAAELTAPAQGIVTFEDVAVYFSWKEWGLLDEAQKCLYHDVMLENLTLTTSLGGSGAGDEEAPYQQSTSPQRVSQVRIPK

SEQ ID NO: 501 - protein, 41 aa, synthetic construct
PLTFQDVAVYFSQAEGRQLGPQQRALYRDVMLENYGNVASL

SEQ ID NO: 502 - protein, 80 aa, synthetic construct
AATFQLPGHQEMPLTFQDVAVYFSQAEGRQLGPQQRALYRDVMLENYGNVASLGFPVPKPELISQLEQGKELWVLNLLGA

SEQ ID NO: 503 - protein, 29 aa, synthetic construct
SEFKPFFGNIIITDVTANCLTVTFKEYVT

SEQ ID NO: 504 - protein, 80 aa, synthetic construct
RSEAGEPPSSLQVKPETPASAAVAVAAAAAPTTTAEKPPAEAQDEPAESLSEFKPFFGNIIITDVTANCLTVTFKEYVTV

SEQ ID NO: 505 - protein, 39 aa, synthetic construct
MCALCGGYFIDATTIVECLHSFCKTCIVRYLETNKYCPM

SEQ ID NO: 506 - protein, 80 aa, synthetic construct
HRTTRIKITELNPHLMCALCGGYFIDATTIVECLHSFCKTCIVRYLETNKYCPMCDVQVHKTRPLLSIRSDKTLQDIVYK

SEQ ID NO: 507 - protein, 51 aa, synthetic construct
FEVEAIVDKRQDKNGNTQYLVRWKGYDKQDDTWEPEQHLMNCEKCVHDFNR

SEQ ID NO: 508 - protein, 80 aa, synthetic construct
ASQEFEVEAIVDKRQDKNGNTQYLVRWKGYDKQDDTWEPEQHLMNCEKCVHDFNRRQTEKQKKLTWTTTSRIFSNNARRR

SEQ ID NO: 509 - protein, 50 aa, synthetic construct
YEVERIVDKRKNKKGKWEYLIRWKGYGSTEDTWEPEHHLLHCEEFIDEFN

SEQ ID NO: 510 - protein, 80 aa, synthetic construct
ASGDLYEVERIVDKRKNKKGKWEYLIRWKGYGSTEDTWEPEHHLLHCEEFIDEFNGLHMSKDKRIKSGKQSSTSKLLRDS

SEQ ID NO: 511 - protein, 41 aa, synthetic construct
SMTFKDVAVDITQEDWELMRPVQKELYKTVTLQNYWNMVSL

SEQ ID NO: 512 - protein, 80 aa, synthetic construct
PRGKHAFQTGWLNDLVTKESMTFKDVAVDITQEDWELMRPVQKELYKTVTLQNYWNMVSLGLTVYRPTVIPILEEPWMVI

SEQ ID NO: 513 - protein, 72 aa, synthetic construct
RKADLENHNKDGGFWTVIDGKVYDIKDFQTQSLTGNSILAQFAGEDPVVALEAALQFEDTRESMHAFCVGQY

SEQ ID NO: 514 - protein, 80 aa, synthetic construct
TLIRKADLENHNKDGGFWTVIDGKVYDIKDFQTQSLTGNSILAQFAGEDPVVALEAALQFEDTRESMHAFCVGQYLEPDQ

SEQ ID NO: 515 - protein, 41 aa, synthetic construct
SVTFDDVAVEFTPEEWALLDTTQKYLYRDVMLENYMNLASV

SEQ ID NO: 516 - protein, 80 aa, synthetic construct
EKTKIGTMVEDHRSNSYQDSVTFDDVAVEFTPEEWALLDTTQKYLYRDVMLENYMNLASVDFFFCLTSEWEIQPRTKRSS

SEQ ID NO: 517 - protein, 41 aa, synthetic construct
LVMFRDVAIDVSQEEWECLNPAQRNLYKEVMLENYSNLVSL

SEQ ID NO: 518 - protein, 80 aa, synthetic construct
AHELVMFRDVAIDVSQEEWECLNPAQRNLYKEVMLENYSNLVSLGLSVSKPAVISSLEQGKEPWMVVREETGRWCPGTWK

SEQ ID NO: 519 - protein, 40 aa, synthetic construct
MAFEDVAVYFSQEEWGLLDTAQRALYRRVMLDNFALVASL

SEQ ID NO: 520 - protein, 80 aa, synthetic construct
AFEDVAVYFSQEEWGLLDTAQRALYRRVMLDNFALVASLGLSTSRPRVVIQLERGEEPWVPSGTDTTLSRTTYRRRNPGS

SEQ ID NO: 521 - protein, 41 aa, synthetic construct
HLTFRDVAIEFSQEEWKCLDPVQKALYRDVMLENYRNLVSL

SEQ ID NO: 522 - protein, 80 aa, synthetic construct
AQLRRGHLTFRDVAIEFSQEEWKCLDPVQKALYRDVMLENYRNLVSLGICLPDLSIISMMKQRTEPWTVENEMKVAKNPD

SEQ ID NO: 523 - protein, 41 aa, synthetic construct
LLYNMNDCYSKLKELVPSIPQNKKVSKMEILQHVIDYILDL

SEQ ID NO: 524 - protein, 80 aa, synthetic construct
SDHSLGISRSKTPVDDPMSLLYNMNDCYSKLKELVPSIPQNKKVSKMEILQHVIDYILDLQIALDSHPTIVSLHHQRPGQ

SEQ ID NO: 525 - protein, 68 aa, synthetic construct
PQKPVSAYALFFRDTQAAIKGQNPNATFGEVSKIVASMWDGLGEEQKQVYKKKTEAAKKEYLKQLAAY

SEQ ID NO: 526 - protein, 80 aa, synthetic construct
KDPNEPQKPVSAYALFFRDTQAAIKGQNPNATFGEVSKIVASMWDGLGEEQKQVYKKKTEAAKKEYLKQLAAYRASLVSK

SEQ ID NO: 527 - protein, 41 aa, synthetic construct
PVTFQDVAVDFSREEWGLLGPTQRTEYRDVMLETFGHLVSV

SEQ ID NO: 528 - protein, 80 aa, synthetic construct
QEEKQEDAAICPVTVLPEEPVTFQDVAVDFSREEWGLLGPTQRTEYRDVMLETFGHLVSVGWETTLENKELAPNSDIPEE

SEQ ID NO: 529 - protein, 41 aa, synthetic construct
LLTFEDMAVYFSEEEWQLLGPLEKTLYNDVMQDIYETAISL

SEQ ID NO: 530 - protein, 80 aa, synthetic construct
KSTKDWKMAPKLIWPESQSLLTFEDMAVYFSEEEWQLLGPLEKTLYNDVMQDIYETAISLGKQRTGKIMGIEMASSFSKE

SEQ ID NO: 531 - protein, 65 aa, synthetic construct
SSWTVEDVMQFVREADPQLGPHADLFRKHEIDGKALLLLRSDMMMKYMGLKLGPALKLSYHIDRL

SEQ ID NO: 532 - protein, 80 aa, synthetic construct
DASRLSGRDPSSWTVEDVMQFVREADPQLGPHADLFRKHEIDGKALLLLRSDMMMKYMGLKLGPALKLSYHIDRLKQGKF

SEQ ID NO: 533 - protein, 41 aa, synthetic construct
SVTFEDTAVDFTQEEWILLDPVQRNLYRDVMLENYENVAKV

SEQ ID NO: 534 - protein, 80 aa, synthetic construct
AYCLTNCYQYSVTFEDTAVDFTQEEWILLDPVQRNLYRDVMLENYENVAKVGFQLFKPSVISWLEEEELRTLQQGVLQDW

SEQ ID NO: 535 - protein, 66 aa, synthetic construct
SAWTVEDVVWFVKDADPQALGPHVELFRKHEIDGNALLLLKSDMVMKYLGLKLGPALKLCYHIDKL

SEQ ID NO: 536 - protein, 80 aa, synthetic construct
ARRPRSRNPSAWTVEDVVWFVKDADPQALGPHVELFRKHEIDGNALLLLKSDMVMKYLGLKLGPALKLCYHIDKLKQAKF

SEQ ID NO: 537 - protein, 41 aa, synthetic construct
AVTFEDVTIIFTWEEWKFLDSSQKRLYREVMWENYTNVMSV

SEQ ID NO: 538 - protein, 80 aa, synthetic construct
AVTFEDVTIIFTWEEWKFLDSSQKRLYREVMWENYTNVMSVENWNESYKSQEEKFRYLEYENFSYWQGWWNAGAQMYENQ

SEQ ID NO: 539 - protein, 49 aa, synthetic construct [sequence omitted]

SEQ ID NO: 540 - protein, 80 aa, synthetic construct
ELSAIGEQVFAVESIRKKRVRKGKVEYLVKWKGWPPKYSTWEPEEHILDPRLVMAYEEKEERDRASGYRKRGPKPKRLLL

SEQ ID NO: 541 - protein, 40 aa, synthetic construct
LYDMNGCYSRLKELVPTLPQNRKVSKVEILQHVIDYIRDL

SEQ ID NO: 542 - protein, 80 aa, synthetic construct
GGAGARLPALLDEQQVNVLLYDMNGCYSRLKELVPTLPQNRKVSKVEILQHVIDYIRDLQLELNSESEVGTPGGRGLPVR

SEQ ID NO: 543 - protein, 45 aa, synthetic construct
AKECRRRKKEYVKCLESRVAVLEVQNKKLIEELETLKDICSPKTD

SEQ ID NO: 544 - protein, 80 aa, synthetic construct
VVMAASPGSLHSPQQLAEEATRKRELRLMKNREAAKECRRRKKEYVKCLESRVAVLEVQNKKLIEELETLKDICSPKTDY

SEQ ID NO: 545 - protein, 52 aa, synthetic construct
QRQAANIRERKRMFNLNEAFDQLRRKVPTFAYEKRLSRIETLRLAIVYISFM

SEQ ID NO: 546 - protein, 80 aa, synthetic construct
SLLGRPKRKRVITYAQRQAANIRERKRMFNLNEAFDQLRRKVPTFAYEKRLSRIETLRLAIVYISFMTELLESCEKKESG

SEQ ID NO: 547 - protein, 52 aa, synthetic construct
QRHTANARERDRTNSVNTAFTALRTLIPTEPADRKLSKIETLRLASSYISHL

SEQ ID NO: 548 - protein, 80 aa, synthetic construct
GGGPGGRPGREPRQRHTANARERDRTNSVNTAFTALRTLIPTEPADRKLSKIETLRLASSYISHLGNVLLAGEACGDGQP

SEQ ID NO: 549 - protein, 52 aa, synthetic construct
AVARRNERERNRVKLVNLGFATLREHVPNGAANKKMSKVETLRSAVEYIRAL

SEQ ID NO: 550 - protein, 80 aa, synthetic construct
SGFGYSLPQQQPAAVARRNERERNRVKLVNLGFATLREHVPNGAANKKMSKVETLRSAVEYIRALQQLLDEHDAVSAAFQ

SEQ ID NO: 551 - protein, 41 aa, synthetic construct
AVSFADVAVYFCREEWGCLRPAQRALYRDVMRETYGHLSAL

SEQ ID NO: 552 - protein, 80 aa, synthetic construct
APLPPRDPNGAGPEWREPGAVSFADVAVYFCREEWGCLRPAQRALYRDVMRETYGHLSALGIGGNKPALISWVEEEAELW

SEQ ID NO: 553 - protein, 66 aa, synthetic construct
STWSVDEVIQFMKHTDPQISGPLADLFRQHEIDGKALFLLKSDVMMKYMGLKLGPALKLCYYIEKL

SEQ ID NO: 554 - protein, 80 aa, synthetic construct
KQGFSKDPSTWSVDEVIQFMKHTDPQISGPLADLFRQHEIDGKALFLLKSDVMMKYMGLKLGPALKLCYYIEKLKEGKYS

SEQ ID NO: 555 - protein, 51 aa, synthetic construct
IQKRNERERQRVKCVNEGYARLRGHLPGALAEKRLSKVETLRAAIRYIKYL

SEQ ID NO: 556 - protein, 80 aa, synthetic construct
GAFGVYEYPFEPAFIQKRNERERQRVKCVNEGYARLRGHLPGALAEKRLSKVETLRAAIRYIKYLQELLSSAPDGSTPPA

SEQ ID NO: 557 - protein, 51 aa, synthetic construct
QRVMANVRERQRTQSLNEAFAALRKIIPTLPSDKLSKIQTLKLAARYIDFL

SEQ ID NO: 558 - protein, 80 aa, synthetic construct
SGGGSPQSYEELQTQRVMANVRERQRTQSLNEAFAALRKIIPTLPSDKLSKIQTLKLAARYIDFLYQVLQSDELDSKMAS

SEQ ID NO: 559 - protein, 25 aa, synthetic construct
DLQRHRRVHTGEKPYKCPNCDKAFK

SEQ ID NO: 560 - protein, 80 aa, synthetic construct
FVQHRCDPAREKPLKCPDCEKRFKYASDLQRHRRVHTGEKPYKCPNCDKAFKQREHLNKHQGVHAREQQFKCVWCGERFL

SEQ ID NO: 561 - protein, 41 aa, synthetic construct
CMVFEDVAIYFSQEEWGILNDAQRHLHSNVMLENFALLSSV

SEQ ID NO: 562 - protein, 80 aa, synthetic construct
NLTEDCMVFEDVAIYFSQEEWGILNDAQRHLHSNVMLENFALLSSVGCWHGAKDEEVPSKQCVSVRVLQVTIPKPALSTL

SEQ ID NO: 563 - protein, 41 aa, synthetic construct
FVTLKDVGMDFTLGDWEQLGLEQGDTFWDTALDNCQDLFLL

SEQ ID NO: 564 - protein, 80 aa, synthetic construct
AEEFVTLKDVGMDFTLGDWEQLGLEQGDTFWDTALDNCQDLFLLDPPRPNLTSHPDGSEDLEPLAGGSPEATSPDVTETK

SEQ ID NO: 565 - protein, 41 aa, synthetic construct
HEKFPSDLDLDMFNGSLECDMESIIRSELMDADGLDFNFDS

SEQ ID NO: 566 - protein, 80 aa, synthetic construct
DSLSGSSLYSTSANLPVMGHEKFPSDLDLDMFNGSLECDMESIIRSELMDADGLDFNFDSLISTQNVVGLNVGNFTGAKQ

SEQ ID NO: 567 - protein, 40 aa, synthetic construct
QEKLPSDLDGMFIERLDCDMESIIRNDLMDGDTLDFNFDN

SEQ ID NO: 568 - protein, 80 aa, synthetic construct
GGYSSVSSCNGYGRMGLLHQEKLPSDLDGMFIERLDCDMESIIRNDLMDGDTLDFNFDNVLPNQSFPHSVKTTTHSWVSG

SEQ ID NO: 569 - protein, 46 aa, synthetic construct
GNCIEHVQPTSAFIQQPFIDEDPDKEKKIKELEMLLMSAENEVRRK

SEQ ID NO: 570 - protein, 80 aa, synthetic construct
FYIPVQIPGYQYVSPEGNCIEHVQPTSAFIQQPFIDEDPDKEKKIKELEMLLMSAENEVRRKRIPSQPGSFSSWSGSFLM

SEQ ID NO: 571 - protein, 46 aa, synthetic construct
VNIVNVPQPAAAAIQRHYNDEDPEKEKRIKELELLLMSTENELKGQ

SEQ ID NO: 572 - protein, 80 aa, synthetic construct
EAQNVSSHVPYPVALHVNIVNVPQPAAAAIQRHYNDEDPEKEKRIKELELLLMSTENELKGQQVLPTQNHTCSYPGWHST

SEQ ID NO: 573 - protein, 46 aa, synthetic construct
ESPSDEGALLDQLYLALRNFDGLEEIDRALGIPELVSQSQAVDPEQ

SEQ ID NO: 574 - protein, 80 aa, synthetic construct
PFGSSPDDLLCPHPAAESPSDEGALLDQLYLALRNFDGLEEIDRALGIPELVSQSQAVDPEQFSSQDSNIMLEQKAPVFP

SEQ ID NO: 575 - protein, 34 aa, synthetic construct
PFSPVQLHQLRAQILAYKMLARGQPLPETLQLAV

SEQ ID NO: 576 - protein, 80 aa, synthetic construct
SQPGALIPGDPQAMSQPNRGPSPFSPVQLHQLRAQILAYKMLARGQPLPETLQLAVQGKRTLPGLQQQQQQQQQQQQQQQ

SEQ ID NO: 577 - protein, 29 aa, synthetic construct
LPEGWEEARDFDGKVYYIDHTNRTTSWID

SEQ ID NO: 578 - protein, 80 aa, synthetic construct
PRPELPLPEGWEEARDFDGKVYYIDHTNRTTSWIDPRDRYTKPLTFADCISDELPLGWEEAYDPQVGDYFIDHNTKTTQI

SEQ ID NO: 579 - protein, 48 aa, synthetic construct
EGQSDERALLDQLHTLLSNTDATGLEEIDRALGIPELVNQGQALEPKQ

SEQ ID NO: 580 - protein, 80 aa, synthetic construct
LRNSLDDLVGPPSNLEGQSDERALLDQLHTLLSNTDATGLEEIDRALGIPELVNQGQALEPKQDAFQGQEAAVMMDQKAG

SEQ ID NO: 581 - protein, 41 aa, synthetic construct
PDRFPADLDLDMFSGSLECDVESIILNDFMDSDEMDFNFDS

SEQ ID NO: 582 - protein, 80 aa, synthetic construct
LDALPGPYAAAAAGPLGAAPDRFPADLDLDMFSGSLECDVESIILNDFMDSDEMDFNFDSALPPPPPGLAGAPPPNQSWV

SEQ ID NO: 583 - protein, 41 aa, synthetic construct
PILFEDLAVYFSQEECVTLHPAQRSLSKDGTKESLEDAALM

SEQ ID NO: 584 - protein, 80 aa, synthetic construct
ASMPPTPEAQGPILFEDLAVYFSQEECVTLHPAQRSLSKDGTKESLEDAALMGEEGKPEINQQLSLESMELDELALEKYP

SEQ ID NO: 585 - protein, 28 aa, synthetic construct
LPAGWMRVQDTSGTYYWHIPTGTTQWEP

SEQ ID NO: 586 - protein, 80 aa, synthetic construct
GSPSYGSPEDTDSFWNPNAFETDSDLPAGWMRVQDTSGTYYWHIPTGTTQWEPPGRASPSQGSSPQEESQLTWTGFAHGE

SEQ ID NO: 587 - protein, 45 aa, synthetic construct
VAIADYAATDETQLSFLRGEKILILRQTTADWWWGERAGCCGYIP

SEQ ID NO: 588 - protein, 80 aa, synthetic construct
ECSEAGLLQEGVQPEEFVAIADYAATDETQLSFLRGEKILILRQTTADWWWGERAGCCGYIPANHVGKHVDEYDPEDTWQ

SEQ ID NO: 589 - protein, 75 aa, synthetic construct
SFLPLVHNIIKCMDKDSPEVHQDLNALKSKFQEMRKLISTMPGIHLSPEQQQQQLQSLREQVRTKNELLQKYKSL

SEQ ID NO: 590 - protein, 80 aa, synthetic construct
NYSFLPLVHNIIKCMDKDSPEVHQDLNALKSKFQEMRKLISTMPGIHLSPEQQQQQLQSLREQVRTKNELLQKYKSLCMF

SEQ ID NO: 591 - protein, 48 aa, synthetic construct
YCICRKPDINCFMIGCDNCNEWFHGDCIRITEKMAKAIREWYCRECRE

SEQ ID NO: 592 - protein, 80 aa, synthetic construct
AGEDSKSENGENAPIYCICRKPDINCFMIGCDNCNEWFHGDCIRITEKMAKAIREWYCRECREKDPKLEIRYRHKKSRER

SEQ ID NO: 593 - protein, 77 aa, synthetic construct
PNIILTGDSSPGFSKEIAAALAGVPGFEVSAAGLELGLGLEDELRMEPLGLEGLNMLSDPCALLPDPAVEESFRSDR

SEQ ID NO: 594 - protein, 80 aa, synthetic construct
GPNIILTGDSSPGFSKEIAAALAGVPGFEVSAAGLELGLGLEDELRMEPLGLEGLNMLSDPCALLPDPAVEESFRSDRLQ

SEQ ID NO: 595 - protein, 34 aa, synthetic construct
WANCSSPLPCWDYINNQCDELCNTVECLFDNFEC

SEQ ID NO: 596 - protein, 80 aa, synthetic construct
EACNSHACQWDGGDCSLTMENPWANCSSPLPCWDYINNQCDELCNTVECLFDNFECQGNSKTCKYDKYCADHFKDNHCDQ

SEQ ID NO: 597 - protein, 26 aa, synthetic construct
KECPICKERFPAESDKDALEDHMDGH

SEQ ID NO: 598 - protein, 80 aa, synthetic construct
SGGEEANLLLPELGSAFYDMASGFTVGTLSETSTGGPATPTWKECPICKERFPAESDKDALEDHMDGHFFFSTQDPFTFE

SEQ ID NO: 599 - protein, 56 aa, synthetic construct
PCGICTNEVNDDQDAILCEASCQKWFHRICTGMTETAYGLLTAEASAVWGCDTCMA

SEQ ID NO: 600 - protein, 80 aa, synthetic construct
RHGHSSSDPVYPCGICTNEVNDDQDAILCEASCQKWFHRICTGMTETAYGLLTAEASAVWGCDTCMADKDVQLMRTRETF

SEQ ID NO: 601 - protein, 36 aa, synthetic construct
AQMIEENLNCLGHLSTIIHEANEEQGNSMMNLDWSW

SEQ ID NO: 602 - protein, 80 aa, synthetic construct
LVGSSLEGAVTPQTSAWLPPTSAEHDHSLSCVVTPQDGETSAQMIEENLNCLGHLSTIIHEANEEQGNSMMNLDWSWLTE

SEQ ID NO: 603 - protein, 79 aa, synthetic construct
DLAPPRKALFTYPKGAGEMLEDGSERFLCESVFSYQVASTLKQVKHDQQVARMEKLAGLVEELEADEWRFKPIEQLLGF

SEQ ID NO: 604 - protein, 80 aa, synthetic construct
DLAPPRKALFTYPKGAGEMLEDGSERFLCESVFSYQVASTLKQVKHDQQVARMEKLAGLVEELEADEWRFKPIEQLLGFT

SEQ ID NO: 605 - protein, 28 aa, synthetic construct
LPPGWEKRQDNGRVYYVNHNTRTTQWED

SEQ ID NO: 606 - protein, 80 aa, synthetic construct
AMQHFSQRFLYQSSSASTDHDPLGPLPPGWEKRQDNGRVYYVNHNTRTTQWEDPRTQGMIQEPALPPGWEMKYTSEGVRY

SEQ ID NO: 607 - protein, 68 aa, synthetic construct
FLVQEVMEEEWNALQSVENCPEDLAQLEELIDMAVLEEIQQELINQEQSIISEYEKSLQFDEKCLSIM

SEQ ID NO: 608 - protein, 80 aa, synthetic construct
NSQNSFLVQEVMEEEWNALQSVENCPEDLAQLEELIDMAVLEEIQQELINQEQSIISEYEKSLQFDEKCLSIMLAEWEAN

SEQ ID NO: 609 - protein, 61 aa, synthetic construct
AYLDELVELHRRLMTLRERHILQQIVNLIEETGHFHITNTTFDFDLCSLDKTTVRKLQSYL

SEQ ID NO: 610 - protein, 80 aa, synthetic construct
KSDKQIKNGECDKAYLDELVELHRRLMTLRERHILQQIVNLIEETGHFHITNTTFDFDLCSLDKTTVRKLQSYLETSGTS

SEQ ID NO: 611 - protein, 37 aa, synthetic construct
LVTIKNLAVDFRQQLHPAQKNFCKNGIWENNSDLGSA

SEQ ID NO: 612 - protein, 80 aa, synthetic construct
LEYSLLGEHWDYDALFETQPGLVTIKNLAVDFRQQLHPAQKNFCKNGIWENNSDLGSAGHCVAKPDLVSLLEQEKEPWMV

SEQ ID NO: 613 - protein, 29 aa, synthetic construct
LPPGWEKRVDSTDRVYFVNHNTKTTQWED

SEQ ID NO: 614 - protein, 80 aa, synthetic construct
AMQQFNQRYLYSASMLAAENDPYGPLPPGWEKRVDSTDRVYFVNHNTKTTQWEDPRTQGLQNEEPLPEGWEIRYTREGVR

SEQ ID NO: 615 - protein, 41 aa, synthetic construct
PTRAYLDQTVVPILLQGLAVLAKERPPNPIEFLASYLLKNK

SEQ ID NO: 616 - protein, 80 aa, synthetic construct
EYGLTDNVERIVENEKINAEKSSKQKVDLQSLPTRAYLDQTVVPILLQGLAVLAKERPPNPIEFLASYLLKNKAQFEDRN

SEQ ID NO: 617 - protein, 40 aa, synthetic construct
LQSEEDVSQFDTRFTRQTPVDSPDDTALSESANQAFLGFT

SEQ ID NO: 618 - protein, 80 aa, synthetic construct
HMNWDDLLAWRVDPPFRPCLQSEEDVSQFDTRFTRQTPVDSPDDTALSESANQAFLGFTYVAPSVLDSIKEGFSFQPKLR

SEQ ID NO: 619 - protein, 56 aa, synthetic construct
PCGACRSEVNDDQDAILCEASCQKWFHRECTGMTESAYGLLTTEASAVWACDLCLK

SEQ ID NO: 620 - protein, 80 aa, synthetic construct
SGPQPPPGLVYPCGACRSEVNDDQDAILCEASCQKWFHRECTGMTESAYGLLTTEASAVWACDLCLKTKEIQSVYIREGM

SEQ ID NO: 621 - protein, 59 aa, synthetic construct
FEEVFTELQEKYGEIEEMNVCDNLGDHLVGNVYVKFRREEDGERAVAELSNRWFNGQAV

SEQ ID NO: 622 - protein, 80 aa, synthetic construct
VEVQEHYDSFFEEVFTELQEKYGEIEEMNVCDNLGDHLVGNVYVKFRREEDGERAVAELSNRWFNGQAVHGELSPVTDFR

SEQ ID NO: 623 - protein, 29 aa, synthetic construct
LPPGWEQRVDQHGRVYYVDHVEKRTTWDR

SEQ ID NO: 624 - protein, 80 aa, synthetic construct
SGLIIPLTISGGSGPRPLNPVTQAPLPPGWEQRVDQHGRVYYVDHVEKRTTWDRPEPLPPGWERRVDNMGRIYYVDHFTR

SEQ ID NO: 625 - protein, 61 aa, synthetic construct
AYTDELVELHRRLMALRERNVLQQIVNLIEETGHFNVTNTTFDFDLFSLDETTVRKLQSCL

SEQ ID NO: 626 - protein, 80 aa, synthetic construct
SKPEKILKKGTYDKAYTDELVELHRRLMALRERNVLQQIVNLIEETGHFNVTNTTFDFDLFSLDETTVRKLQSCLEAVAT

SEQ ID NO: 627 - protein, 55 aa, synthetic construct
SQPVPEPDLPCDLRHLNTEPMEIFRNCVKIEEIMPNGDPLLAGQNTVDEVYVSRP

SEQ ID NO: 628 - protein, 80 aa, synthetic construct
SQTVPEPDQGPVSQPVPEPDLPCDLRHLNTEPMEIFRNCVKIEEIMPNGDPLLAGQNTVDEVYVSRPSHFYTDGPLMPSD

SEQ ID NO: 629 - protein, 34 aa, synthetic construct
EEACELPECQEDAGNKVCSLQCNNHACGWDGGDC

SEQ ID NO: 630 - protein, 80 aa, synthetic construct
LCHILDYSFGGGAGRDIPPPLIEEACELPECQEDAGNKVCSLQCNNHACGWDGGDCSLNFNDPWKNCTQSLQCWKYFSDG

SEQ ID NO: 631 - protein, 74 aa, synthetic construct
PNTILPEDSSTSLFKDLNSALAGLPEVSLNVDTPFPLEEELQIEPLSLDGLNMLSDSSMGLLDPSVEETFRADR

SEQ ID NO: 632 - protein, 80 aa, synthetic construct
NCGSLPNTILPEDSSTSLFKDLNSALAGLPEVSLNVDTPFPLEEELQIEPLSLDGLNMLSDSSMGLLDPSVEETFRADRL

SEQ ID NO: 633 - protein, 29 aa, synthetic construct
LPPGWSVDWTMRGRKYYIDHNTNTTHWSH

SEQ ID NO: 634 - protein, 80 aa, synthetic construct
HASGIGRVAATSLGNLTNHGSEDLPLPPGWSVDWTMRGRKYYIDHNTNTTHWSHPLEREGLPPGWERVESSEFGTYYVDH

SEQ ID NO: 635 - protein, 71 aa, synthetic construct
GEDFYREAIEHCRSYNARLCAERSLRLPFLDSQTGVAQNNCYIWMEKTHRGPGLAPGQIYTYPARCWRKKR

SEQ ID NO: 636 - protein, 80 aa, synthetic construct
PLSLGEDFYREAIEHCRSYNARLCAERSLRLPFLDSQTGVAQNNCYIWMEKTHRGPGLAPGQIYTYPARCWRKKRRLNIL

SEQ ID NO: 637 - protein, 46 aa, synthetic construct
VALYDFVASGDNTLSITKGEKLRVLGYNHNGEWCEAQTKNGQGWVP

SEQ ID NO: 638 - protein, 80 aa, synthetic construct
KENLLAGPSENDPNLFVALYDFVASGDNTLSITKGEKLRVLGYNHNGEWCEAQTKNGQGWVPSNYITPVNSLEKHSWYHG

SEQ ID NO: 639 - protein, 29 aa, synthetic construct
VKTVWVEGLSEDGFTYYYNTETGESRWEK

SEQ ID NO: 640 - protein, 80 aa, synthetic construct
YYDLISGASQWEKPEGFQGDLKKTAVKTVWVEGLSEDGFTYYYNTETGESRWEKPDDFIPHTSDLPSSKVNENSLGTLDE

SEQ ID NO: 641 - protein, 46 aa, synthetic construct
VALYDYMPMNANDLQLRKGDEYFILEESNLPWWRARDKNGQEGYIP

SEQ ID NO: 642 - protein, 80 aa, synthetic construct
PEPAAAPVSTSELKKVVALYDYMPMNANDLQLRKGDEYFILEESNLPWWRARDKNGQEGYIPSNYVTEAEDSIEMYEWYS

SEQ ID NO: 643 - protein, 45 aa, synthetic construct
TREWTEQETLLLLEALEMYKDDWNKVSEHVGSRTQDECILHFLRL

SEQ ID NO: 644 - protein, 80 aa, synthetic construct
MYTKKNVPSKSKAAASATREWTEQETLLLLEALEMYKDDWNKVSEHVGSRTQDECILHFLRLPIEDPYLEDSEASLGPLA

SEQ ID NO: 645 - protein, 37 aa, synthetic construct
CESCYATQSHQWYSWGPPNMQCRLCAICWLYWKKYGG

SEQ ID NO: 646 - protein, 80 aa, synthetic construct
GAVNGAVGTTFQPQNPLLGRACESCYATQSHQWYSWGPPNMQCRLCAICWLYWKKYGGLKMPTQSEEEKLSPSPTTEDPR

SEQ ID NO: 647 - protein, 29 aa, synthetic construct
LPPGWEMTFTATGQRYFLNHIEKITTWQD

SEQ ID NO: 648 - protein, 80 aa, synthetic construct
GAAGSPAQQHAHLRQQSYDVTDELPLPPGWEMTFTATGQRYFLNHIEKITTWQDPRKAMNQPLNHMNLHPAVSSTPVPQR

SEQ ID NO: 649 - protein, 69 aa, synthetic construct
GKFAFDSPSNWCQDNIISLMSAGILGVPPASGALSTQTSTASMVQPPQGDVEAMYPALPPYSNCGDLYS

SEQ ID NO: 650 - protein, 80 aa, synthetic construct
TVTYLGKFAFDSPSNWCQDNIISLMSAGILGVPPASGALSTQTSTASMVQPPQGDVEAMYPALPPYSNCGDLYSEPVSFH

SEQ ID NO: 651 - protein, 39 aa, synthetic construct
LTQDEFHPFIEALLPHVRAFSYTWFNLQARKRKYFKKHE

SEQ ID NO: 652 - protein, 80 aa, synthetic construct
YSPYCLTQDEFHPFIEALLPHVRAFSYTWFNLQARKRKYFKKHEKRMSKDEERAVKDELLGEKPEIKQKWASRLLAKLRK

SEQ ID NO: 653 - protein, 40 aa, synthetic construct
SGEFGLDNFDSQFTNEPVQLTPDDDDIVRKIDQSEFEGFE

SEQ ID NO: 654 - protein, 80 aa, synthetic construct
QGHPFFRNVDWDMMEQKQVVPPFKPNISGEFGLDNFDSQFTNEPVQLTPDDDDIVRKIDQSEFEGFEYINPLLMSAEECV

SEQ ID NO: 655 - protein, 68 aa, synthetic construct
MKLEAVDRRNPALIRVASVEDVEDHRIKIHFDGWSHGYDFWIDADHPDIHPAGWCSKTGHPLQPPLGP

SEQ ID NO: 656 - protein, 80 aa, synthetic construct
SFLVNMKLEAVDRRNPALIRVASVEDVEDHRIKIHFDGWSHGYDFWIDADHPDIHPAGWCSKTGHPLQPPLGPREPSSAS

SEQ ID NO: 657 - protein, 35 aa, synthetic construct
WKNCTQSLQCWKYFSDGHCDSQCNSAGCLFDGFDC

SEQ ID NO: 658 - protein, 80 aa, synthetic construct
LQCNNHACGWDGGDCSLNFNDPWKNCTQSLQCWKYFSDGHCDSQCNSAGCLFDGFDCQRAEGQCNPLYDQYCKDHFSDGH

SEQ ID NO: 659 - protein, 45 aa, synthetic construct
TDLSAIMPSQLRPVTYQDFENAFCKIQPSISQKELDMYVEWNKMF

SEQ ID NO: 660 - protein, 80 aa, synthetic construct
LLVQRTEGFSGLDVAHLCQEAVVGPLHAMPATDLSAIMPSQLRPVTYQDFENAFCKIQPSISQKELDMYVEWNKMFGCSQ

SEQ ID NO: 661 - protein, 51 aa, synthetic construct
NPYCALIEEAYGLDKIEFLQSHENQEIYQKAFDLIEHYFGTEDEDSSIAPQ

SEQ ID NO: 662 - protein, 80 aa, synthetic construct
RLGEQEAKRNGTGINPYCALIEEAYGLDKIEFLQSHENQEIYQKAFDLIEHYFGTEDEDSSIAPQVDLNQQQYIFQQCEA

SEQ ID NO: 663 - protein, 34 aa, synthetic construct
PFKDMILCFSEEDWSLLDPAQTGFYGEFIIGEDY

SEQ ID NO: 664 - protein, 80 aa, synthetic construct
QEENVRDTQQVTTLQLPPSRVSPFKDMILCFSEEDWSLLDPAQTGFYGEFIIGEDYGVSMPPNDLAAQPDLSQGEENEPR

SEQ ID NO: 665 - protein, 71 aa, synthetic construct
DAKSLEEESRTLVTFKDVFVDFTREEWKLLDTAQQIVYRNVMLENYKNLVSLGYQLTKPDVILRLEKGEEP

SEQ ID NO: 666 - protein, 71 aa, synthetic construct
DAKSLTAEEETLVTFKDVFVDFTREEWKLLDTAQQIVYRNVMLENYKNLVSLGYQLTKPDVILRLEKGEEP

SEQ ID NO: 667 - protein, 71 aa, synthetic construct
DAKSLTADDDTLVTFKDVFVDFTREEWKLLDTAQQIVYRNVMLENYKNLVSLGYQLTKPDVILRLEKGEEP

SEQ ID NO: 668 - protein, 71 aa, synthetic construct
DAKSLTAWSRTLVTFKDVFVDFTREEWKLLDTAQQIVYRNVMLENYKNLVSLGYQLTKPDVILRLEKIIIP

SEQ ID NO: 669 - protein, 71 aa, synthetic construct
DAKSLTPPPRTLVTFKDVFVDFTREEWKLLDTAQQIVYRNVMLENYKNLVSLGYQLTKPDVILRLEKGEEP

SEQ ID NO: 670 - protein, 71 aa, synthetic construct
DNKSLTAWSRTLVTFKDVFVDFTREEWKLLDTAQQIVYRNVMLENYKNLVSLGYQLTKPDVILRLEKGEEP

SEQ ID NO: 671 - protein, 71 aa, synthetic construct
DAKSLTAWSPPPVTFKDVFVDFTREEWKLLDTAQQIVYRNVMLENYKNLVSLGYQLTKPDVILRLEKGEEP

SEQ ID NO: 672 - protein, 71 aa, synthetic construct
DAKSLTEESRTLVTFKDVFVDFTREEWKLLDTAQQIVYRNVMLENYKNLVSLGYQLTKPDVILRLEKGEEP

SEQ ID NO: 673 - protein, 71 aa, synthetic construct
DAKSLTAAARTLVTFKDVFVDFTREEWKLLDTAQQIVYRNVMLENYKNLVSLGYQLTKPDVILRLEKGEEP

SEQ ID NO: 674 - protein, 71 aa, synthetic construct
DAKSVTAWSRTLVTFKDVFVDFTREEWKLLDTAQQIVYRNVMLENYKNLVSLGYQLTKPDVILRLEKGEEP

SEQ ID NO: 675 - protein, 71 aa, synthetic construct
DAKSLTAPSRTLVTFKDVFVDFTREEWKLLDTAQQIVYRNVMLENYKNLVSLGYQLTKPDVILRLEKGEEP

SEQ ID NO: 676 - protein, 71 aa, synthetic construct
DAKSLTAGGRTLVTFKDVFVDFTREEWKLLDTAQQIVYRNVMLENYKNLVSLGYQLTKPDVILRLEKGEEP

SEQ ID NO: 677 - protein, 71 aa, synthetic construct
DAKSLNNNSRTLVTFKDVFVDFTREEWKLLDTAQQIVYRNVMLENYKNLVSLGYQLTKPDVILRLEKGEEP

SEQ ID NO: 678 - protein, 71 aa, synthetic construct
DAKSLTAQQQTLVTFKDVFVDFTREEWKLLDTAQQIVYRNVMLENYKNLVSLGYQLTKPDVILRLEKGEEP

SEQ ID NO: 679 - protein, 71 aa, synthetic construct
DAKSLTAWSRPPVTFKDVFVDFTREEWKLLDTAQQIVYRNVMLENYKNLVSLGYQLTKPDVILRLEKGEEP

SEQ ID NO: 680 - protein, 71 aa, synthetic construct
DAKSLTATTRTLVTFKDVFVDFTREEWKLLDTAQQIVYRNVMLENYKNLVSLGYQLTKPDVILRLEKGEEP

SEQ ID NO: 681 - protein, 71 aa, synthetic construct
DAKSLTAWSREEVTFKDVFVDFTREEWKLLDTAQQIVYRNVMLENYKNLVSLGYQLTKPDVILRLEKGEEP

SEQ ID NO: 682 - protein, 71 aa, synthetic construct
DAKSLTADDRTLVTFKDVFVDFTREEWKLLDTAQQIVYRNVMLENYKNLVSLGYQLTKPDVILRLEKGEEP

SEQ ID NO: 683 - protein, 71 aa, synthetic construct
DAKSLTQQQRTLVTFKDVFVDFTREEWKLLDTAQQIVYRNVMLENYKNLVSLGYQLTKPDVILRLEKGEEP

SEQ ID NO: 684 - protein, 71 aa, synthetic construct
DAKSEEEWSRTLVTFKDVFVDFTREEWKLLDTAQQIVYRNVMLENYKNLVSLGYQLTKPDVILRLEKGEEP

SEQ ID NO: 685 - protein, 71 aa, synthetic construct
DAKSLTDDSRTLVTFKDVFVDFTREEWKLLDTAQQIVYRNVMLENYKNLVSLGYQLTKPDVILRLEKGEEP

SEQ ID NO: 686 - protein, 71 aa, synthetic construct
DAKSLTANNRTLVTFKDVFVDFTREEWKLLDTAQQIVYRNVMLENYKNLVSLGYQLTKPDVILRLEKGEEP

SEQ ID NO: 687 - protein, 71 aa, synthetic construct
NNNSLTAWSRTLVTFKDVFVDFTREEWKLLDTAQQIVYRNVMLENYKNLVSLGYQLTKPDVILRLEKGEEP

SEQ ID NO: 688 - protein, 71 aa, synthetic construct
DAKSLGGGSRTLVTFKDVFVDFTREEWKLLDTAQQIVYRNVMLENYKNLVSLGYQLTKPDVILRLEKGEEP

SEQ ID NO: 689 - protein, 71 aa, synthetic construct
DAKSLTDDDRTLVTFKDVFVDFTREEWKLLDTAQQIVYRNVMLENYKNLVSLGYQLTKPDVILRLEKGEEP

SEQ ID NO: 690 - protein, 71 aa, synthetic construct
DAKSLDDDSRTLVTFKDVFVDFTREEWKLLDTAQQIVYRNVMLENYKNLVSLGYQLTKPDVILRLEKGEEP

SEQ ID NO: 691 - protein, 71 aa, synthetic construct
DAKSLTAWSRTLVTFKDVFVDFTREEWKLLDTAQQIVYRNVMLENYKNLVSLGYQLTKPDVIKKLEKGEEP

SEQ ID NO: 692 - protein, 71 aa, synthetic construct
DAKSLTADSRTLVTFKDVFVDFTREEWKLLDTAQQIVYRNVMLENYKNLVSLGYQLTKPDVILRLEKGEEP

SEQ ID NO: 693 - protein, 71 aa, synthetic construct
DAKSLKKKSRTLVTFKDVFVDFTREEWKLLDTAQQIVYRNVMLENYKNLVSLGYQLTKPDVILRLEKGEEP

SEQ ID NO: 694 - protein, 71 aa, synthetic construct
DAKSLTAESRTLVTFKDVFVDFTREEWKLLDTAQQIVYRNVMLENYKNLVSLGYQLTKPDVILRLEKGEEP

SEQ ID NO: 695 - protein, 71 aa, synthetic construct
DAKSLTAPPRTLVTFKDVFVDFTREEWKLLDTAQQIVYRNVMLENYKNLVSLGYQLTKPDVILRLEKGEEP

SEQ ID NO: 696 - protein, 71 aa, synthetic construct
DAKSLTAPPPTLVTFKDVFVDFTREEWKLLDTAQQIVYRNVMLENYKNLVSLGYQLTKPDVILRLEKGEEP

SEQ ID NO: 697 - protein, 71 aa, synthetic construct
DAKSLTAWDDTLVTFKDVFVDFTREEWKLLDTAQQIVYRNVMLENYKNLVSLGYQLTKPDVILRLEKGEEP

SEQ ID NO: 698 - protein, 71 aa, synthetic construct
DAKSLTANNNTLVTFKDVFVDFTREEWKLLDTAQQIVYRNVMLENYKNLVSLGYQLTKPDVILRLEKGEEP

SEQ ID NO: 699 - protein, 71 aa, synthetic construct
DAKSLTAMSRTLVTFKDVFVDFTREEWKLLDTAQQIVYRNVMLENYKNLVSLGYQLTKPDVILRLEKGEEP

SEQ ID NO: 700 - protein, 71 aa, synthetic construct
DAKSLTAWNNNLVTFKDVFVDFTREEWKLLDTAQQIVYRNVMLENYKNLVSLGYQLTKPDVILRLEKGEEP

SEQ ID NO: 701 - protein, 71 aa, synthetic construct
DAKSLTAWSNNLVTFKDVFVDFTREEWKLLDTAQQIVYRNVMLENYKNLVSLGYQLTKPDVILRLEKGEEP

SEQ ID NO: 702 - protein, 71 aa, synthetic construct
DAKSLTPPSRTLVTFKDVFVDFTREEWKLLDTAQQIVYRNVMLENYKNLVSLGYQLTKPDVILRLEKGEEP

SEQ ID NO: 703 - protein, 71 aa, synthetic construct
DAKSLTEEERTLVTFKDVFVDFTREEWKLLDTAQQIVYRNVMLENYKNLVSLGYQLTKPDVILRLEKGEEP

SEQ ID NO: 704 - protein, 71 aa, synthetic construct
DAKSLAAASRTLVTFKDVFVDFTREEWKLLDTAQQIVYRNVMLENYKNLVSLGYQLTKPDVILRLEKGEEP

SEQ ID NO: 705 - protein, 71 aa, synthetic construct
DAKSLTAQQRTLVTFKDVFVDFTREEWKLLDTAQQIVYRNVMLENYKNLVSLGYQLTKPDVILRLEKGEEP

SEQ ID NO: 706 - protein, 71 aa, synthetic construct
DAKSLTAWSEEEVTFKDVFVDFTREEWKLLDTAQQIVYRNVMLENYKNLVSLGYQLTKPDVILRLEKGEEP

SEQ ID NO: 707 - protein, 71 aa, synthetic construct
DAKSLTQQSRTLVTFKDVFVDFTREEWKLLDTAQQIVYRNVMLENYKNLVSLGYQLTKPDVILRLEKGEEP

SEQ ID NO: 708 - protein, 71 aa, synthetic construct
DAKSLTAWSRTLVTFKDVFVDFTREEWKLLDTAQQIVYREVMLENYKNLVSLGYQLTKPDVILRLEKGEEP

SEQ ID NO: 709 - protein, 71 aa, synthetic construct
DAKSLTAWSRPLVTFKDVFVDFTREEWKLLDTAQQIVYRNVMLENYKNLVSLGYQLTKPDVILRLEKGEEP

SEQ ID NO: 710 - protein, 71 aa, synthetic construct
DAKSTTAWSRTLVTFKDVFVDFTREEWKLLDTAQQIVYRNVMLENYKNLVSLGYQLTKPDVILRLEKGEEP

SEQ ID NO: 711 - protein, 71 aa, synthetic construct
DAKSLTAWSNNNVTFKDVFVDFTREEWKLLDTAQQIVYRNVMLENYKNLVSLGYQLTKPDVILRLEKGEEP

SEQ ID NO: 712 - protein, 71 aa, synthetic construct
DAKSLTAWDDDLVTFKDVFVDFTREEWKLLDTAQQIVYRNVMLENYKNLVSLGYQLTKPDVILRLEKGEEP

SEQ ID NO: 713 - protein, 71 aa, synthetic construct
DAKSLTAASRTLVTFKDVFVDFTREEWKLLDTAQQIVYRNVMLENYKNLVSLGYQLTKPDVILRLEKGEEP

SEQ ID NO: 714 - protein, 71 aa, synthetic construct
DAKSLTAGSRTLVTFKDVFVDFTREEWKLLDTAQQIVYRNVMLENYKNLVSLGYQLTKPDVILRLEKGEEP

SEQ ID NO: 715 - protein, 71 aa, synthetic construct
DAKSLTASSRTLVTFKDVFVDFTREEWKLLDTAQQIVYRNVMLENYKNLVSLGYQLTKPDVILRLEKGEEP

SEQ ID NO: 716 - protein, 71 aa, synthetic construct
DAKSLTSSSRTLVTFKDVFVDFTREEWKLLDTAQQIVYRNVMLENYKNLVSLGYQLTKPDVILRLEKGEEP

SEQ ID NO: 717 - protein, 71 aa, synthetic construct
DAKDDTAWSRTLVTFKDVFVDFTREEWKLLDTAQQIVYRNVMLENYKNLVSLGYQLTKPDVILRLEKGEEP

SEQ ID NO: 718 - protein, 71 aa, synthetic construct
DAKSLTAWSRTLVTFKDVFVDFTREEWKLLDTAQQIVYRNVMLENYKNLVSLGYQHTKPDVILRLEKGEEP

SEQ ID NO: 719 - protein, 71 aa, synthetic construct
DAKSLTKKSRTLVTFKDVFVDFTREEWKLLDTAQQIVYRNVMLENYKNLVSLGYQLTKPDVILRLEKGEEP

SEQ ID NO: 720 - protein, 71 aa, synthetic construct
DAKSLTAWSEELVTFKDVFVDFTREEWKLLDTAQQIVYRNVMLENYKNLVSLGYQLTKPDVILRLEKGEEP

SEQ ID NO: 721 - protein, 71 aa, synthetic construct
DAKSLTAWQQQLVTFKDVFVDFTREEWKLLDTAQQIVYRNVMLENYKNLVSLGYQLTKPDVILRLEKGEEP

SEQ ID NO: 722 - protein, 71 aa, synthetic construct
DAKSLTAWSRTTVTFKDVFVDFTREEWKLLDTAQQIVYRNVMLENYKNLVSLGYQLTKPDVILRLEKGEEP

SEQ ID NO: 723 - protein, 71 aa, synthetic construct
DAKSLTANSRTLVTFKDVFVDFTREEWKLLDTAQQIVYRNVMLENYKNLVSLGYQLTKPDVILRLEKGEEP

SEQ ID NO: 724 - protein, 71 aa, synthetic construct
DAKSLQQQSRTLVTFKDVFVDFTREEWKLLDTAQQIVYRNVMLENYKNLVSLGYQLTKPDVILRLEKGEEP

SEQ ID NO: 725 - protein, 71 aa, synthetic construct
DAKSLTAWPPTLVTFKDVFVDFTREEWKLLDTAQQIVYRNVMLENYKNLVSLGYQLTKPDVILRLEKGEEP

SEQ ID NO: 726 - protein, 71 aa, synthetic construct
DAKEEEAWSRTLVTFKDVFVDFTREEWKLLDTAQQIVYRNVMLENYKNLVSLGYQLTKPDVILRLEKGEEP

SEQ ID NO: 727 - protein, 71 aa, synthetic construct
DAKSLTNNSRTLVTFKDVFVDFTREEWKLLDTAQQIVYRNVMLENYKNLVSLGYQLTKPDVILRLEKGEEP

SEQ ID NO: 728 - protein, 71 aa, synthetic construct
DAKSLTAWSRTDVTFKDVFVDFTREEWKLLDTAQQIVYRNVMLENYKNLVSLGYQLTKPDVILRLEKGEEP

SEQ ID NO: 729 - protein, 71 aa, synthetic construct
DAKSLTATTTTLVTFKDVFVDFTREEWKLLDTAQQIVYRNVMLENYKNLVSLGYQLTKPDVILRLEKGEEP

SEQ ID NO: 730 - protein, 71 aa, synthetic construct
DAKSLTAWSDDLVTFKDVFVDFTREEWKLLDTAQQIVYRNVMLENYKNLVSLGYQLTKPDVILRLEKGEEP

SEQ ID NO: 731 - protein, 71 aa, synthetic construct
DAKSLTAKSRTLVTFKDVFVDFTREEWKLLDTAQQIVYRNVMLENYKNLVSLGYQLTKPDVILRLEKGEEP

SEQ ID NO: 732 - protein, 71 aa, synthetic construct
DAKSLTAWSRTLVTFEDVFVDFTREEWKLLDTAQQIVYRNVMLENYKNLVSLGYQLTKPDVILRLEKGEEP

SEQ ID NO: 733 - protein, 71 aa, synthetic construct
DAKSLTAWSRTLVTFDDVFVDFTREEWKLLDTAQQIVYRNVMLENYKNLVSLGYQLTKPDVILRLEKGEEP

SEQ ID NO: 734 - protein, 71 aa, synthetic construct; identical to SEQ ID NO: 733

SEQ ID NO: 735 - protein, 71 aa, synthetic construct
DAKSLTAWSQQQVTFKDVFVDFTREEWKLLDTAQQIVYRNVMLENYKNLVSLGYQLTKPDVILRLEKGEEP

SEQ ID NO: 736 - protein, 71 aa, synthetic construct
DAKSLTAWSRTLVTFKDVFVDFTREEWKLLDTAQQIVYRNVMLENYKNLVELGYQLTKPDVILRLEKGEEP

SEQ ID NO: 737 - protein, 71 aa, synthetic construct
DAKSLTAWEEELVTFKDVFVDFTREEWKLLDTAQQIVYRNVMLENYKNLVSLGYQLTKPDVILRLEKGEEP

SEQ ID NO: 738 - protein, 71 aa, synthetic construct
DAKSLTAWSRTLVTFKDVFVDFTREEWKLLDTAQQEVYRNVMLENYKNLVSLGYQLTKPDVILRLEKGEEP

SEQ ID NO: 739 - protein, 71 aa, synthetic construct
DAKSLTAWSRTPVTFKDVFVDFTREEWKLLDTAQQIVYRNVMLENYKNLVSLGYQLTKPDVILRLEKGEEP

SEQ ID NO: 740 - protein, 71 aa, synthetic construct
DAKSLTAWSRTLVTFKDVFVDFTREEWKLLDTAQQIVYRNVMLENYKNLVSLGYEEEKPDVILRLEKGEEP

SEQ ID NO: 741 - protein, 71 aa, synthetic construct
DAKSLTAWSRTLVTFKDVFVDFTREEWKLLDTAQQDDYRNVMLENYKNLVSLGYQLTKPDVILRLEKGEEP

SEQ ID NO: 742 - protein, 71 aa, synthetic construct; identical to SEQ ID NO: 673

SEQ ID NO: 743 - protein, 71 aa, synthetic construct
DAKSLTAQSRTLVTFKDVFVDFTREEWKLLDTAQQIVYRNVMLENYKNLVSLGYQLTKPDVILRLEKGEEP

SEQ ID NO: 744 - protein, 71 aa, synthetic construct
DAKSLTAAAATLVTFKDVFVDFTREEWKLLDTAQQIVYRNVMLENYKNLVSLGYQLTKPDVILRLEKGEEP

SEQ ID NO: 745 - protein, 71 aa, synthetic construct
DAKSLKKWSRTLVTFKDVFVDFTREEWKLLDTAQQIVYRNVMLENYKNLVSLGYQLTKPDVILRLEKGEEP

SEQ ID NO: 746 - protein, 71 aa, synthetic construct
DAKSLTAGGGTLVTFKDVFVDFTREEWKLLDTAQQIVYRNVMLENYKNLVSLGYQLTKPDVILRLEKGEEP

SEQ ID NO: 747 - protein, 71 aa, synthetic construct
DDDSLTAWSRTLVTFKDVFVDFTREEWKLLDTAQQIVYRNVMLENYKNLVSLGYQLTKPDVILRLEKGEEP

SEQ ID NO: 748 - protein, 71 aa, synthetic construct
DAKSLTAWSRTLVTFKDVFVDFTREEWKLLDTQQQIVYRNVMLENYKNLVSLGYQLTKPDVILRLEKGEEP

SEQ ID NO: 749 - protein, 71 aa, synthetic construct
DAKSLTAWSRTLVTFKDVFVDFTREEWKLLDTAQQIVYRNVMLENYKNLVSLGYQLTKPDVILRLEKGEYY

SEQ ID NO: 750 - protein, 71 aa, synthetic construct
DAKSLTAEERTLVTFKDVFVDFTREEWKLLDTAQQIVYRNVMLENYKNLVSLGYQLTKPDVILRLEKGEEP

SEQ ID NO: 751 - protein, 71 aa, synthetic construct
DAKSLTAHSRTLVTFKDVFVDFTREEWKLLDTAQQIVYRNVMLENYKNLVSLGYQLTKPDVILRLEKGEEP

SEQ ID NO: 752 - protein, 71 aa, synthetic construct
DAKSLRRWSRTLVTFKDVFVDFTREEWKLLDTAQQIVYRNVMLENYKNLVSLGYQLTKPDVILRLEKGEEP

SEQ ID NO: 753 - protein, 71 aa, synthetic construct
DAKSLTAWSSSSVTFKDVFVDFTREEWKLLDTAQQIVYRNVMLENYKNLVSLGYQLTKPDVILRLEKGEEP

SEQ ID NO: 754 - protein, 71 aa, synthetic construct
DAKSLTAWSRTLVTFKDVFVDFTREEWKLLDTAQQIVYRNVMLENYKNLVSLGWQLTKPDVILRLEKGEEP

SEQ ID NO: 755 - protein, 71 aa, synthetic construct
DSSSLTAWSRTLVTFKDVFVDFTREEWKLLDTAQQIVYRNVMLENYKNLVSLGYQLTKPDVILRLEKGEEP

SEQ ID NO: 756 - protein, 71 aa, synthetic construct
DAKSLTAWSRTLVTFKDVFVDFTREEWKLLDTAQKKVYRNVMLENYKNLVSLGYQLTKPDVILRLEKGEEP

SEQ ID NO: 757 - protein, 71 aa, synthetic construct
DAKSLTAKKKTLVTFKDVFVDFTREEWKLLDTAQQIVYRNVMLENYKNLVSLGYQLTKPDVILRLEKGEEP

SEQ ID NO: 758 - protein, 71 aa, synthetic construct
DAKSLTAWSGGGVTFKDVFVDFTREEWKLLDTAQQIVYRNVMLENYKNLVSLGYQLTKPDVILRLEKGEEP

SEQ ID NO: 759 - protein, 71 aa, synthetic construct
DAKSLTAWSDDDVTFKDVFVDFTREEWKLLDTAQQIVYRNVMLENYKNLVSLGYQLTKPDVILRLEKGEEP

SEQ ID NO: 760 - protein, 71 aa, synthetic construct
DAKTTTAWSRTLVTFKDVFVDFTREEWKLLDTAQQIVYRNVMLENYKNLVSLGYQLTKPDVILRLEKGEEP

SEQ ID NO: 761 - protein, 71 aa, synthetic construct
DAKSLTAWGGGLVTFKDVFVDFTREEWKLLDTAQQIVYRNVMLENYKNLVSLGYQLTKPDVILRLEKGEEP

SEQ ID NO: 762 - protein, 71 aa, synthetic construct
DAKSLTAWSRELVTFKDVFVDFTREEWKLLDTAQQIVYRNVMLENYKNLVSLGYQLTKPDVILRLEKGEEP

SEQ ID NO: 763 - protein, 71 aa, synthetic construct
DAKSDDDWSRTLVTFKDVFVDFTREEWKLLDTAQQIVYRNVMLENYKNLVSLGYQLTKPDVILRLEKGEEP

SEQ ID NO: 764 - protein, 71 aa, synthetic construct
DAKSLSSSSRTLVTFKDVFVDFTREEWKLLDTAQQIVYRNVMLENYKNLVSLGYQLTKPDVILRLEKGEEP

SEQ ID NO: 765 - protein, 71 aa, synthetic construct
DAKSLTASSSTLVTFKDVFVDFTREEWKLLDTAQQIVYRNVMLENYKNLVSLGYQLTKPDVILRLEKGEEP

SEQ ID NO: 766 - protein, 80 aa, synthetic construct
MMTAVSLTTRPQESVAFEDVAVYFTTKEWAIMVPAERALYRDVMLENYEAVAFVVPPTSKPALVSHLEQGKESCFTQPQG

SEQ ID NO: 767 - protein, 80 aa, synthetic construct
MAHELVMFRDVAIDVSQEEWECLNPAQRNLYKEVMLENYSNLVSLGLSVSKPAVISSLEQGKEPWMVVREETGRWCPGTW

SEQ ID NO: 768 - protein, 80 aa, synthetic construct
MSCSGAGGITAFVAFRDVAVYFTQEEWRLLSPAQRTLHREVMLETYNHLVSLEIPSSKPKLIAQLERGEAPWREERKCPL

SEQ ID NO: 769 - protein, 80 aa, synthetic construct
MDSVVFEDVAVDFTLEEWALLDSAQRDLYRDVMLETFRNLASVDDGTQFKANGSVSLQDMYGQEKSKEQTIPNFTGNNSC

SEQ ID NO: 770 - protein, 80 aa, synthetic construct
NDIARGFERGLEPEKIIGATDSCGDLMFLMKWKDTDEADLVLAKEANVKCPQIVIAFYEERLTWHAYPEDAENKEKETAK

SEQ ID NO: 771 - protein, 80 aa, synthetic construct; identical to SEQ ID NO: 438

SEQ ID NO: 772 - protein, 80 aa, synthetic construct
KKKKEESEKPRGFARGLEPERIIGATDSSGELMFLMKWKNSDEADLVPAKEANVKCPQVVISFYEERLTWHSYPSEDDDK

SEQ ID NO: 773 - protein, 80 aa, synthetic construct
PVTVPVATTSVEELQGAYENEVSKEGLAESEPMICHTLPLPEGFQVVKVGANGEVETLEQGELPPQEDPSWQKDPDYQPP

SEQ ID NO: 774 - protein, 80 aa, synthetic construct
PPSEANSIQSANATTKTSETNHTSRPRLKNVDRSTAQQLAVTVGNVTVIITDFKEKTRSSSTSSSTVTSSAGSEQQNQSS

SEQ ID NO: 775 - protein, 80 aa, synthetic construct
MASVQASRRQWCYLCDLPKMPWAMVWDFSEAVCRGCVNFEGADRIELLIDAARQLKRSHVLPEGRSPGPPALKHPATKDL

SEQ ID NO: 776 - protein, 80 aa, synthetic construct
PHTSANLVMTPQGQLLTLKGPLFSGPVVAVSPDLLESDLKPQVAGSAVALPENDDLFMMPRIVNVTSLATEGGLVDMGGS

SEQ ID NO: 777 - protein, 80 aa, synthetic construct
ELFLQEPPAPDVLQAAGEWEPAAQPPEEEADADLAEGPPPWTPALPSSEVTVTDITANSITVTFREAQAAEGFFRDRSGK

SEQ ID NO: 778 - protein, 80 aa, synthetic construct
TPSIGNSQPSTPAPALPVQDPQLLHHCQHCDMYFADNILYTIHMGCHGYENPFQCNICGCKCKNKYDFACHFARGQHNQH

SEQ ID NO: 779 - protein, 80 aa, synthetic construct
IHSHEGSDLSDNMSEGSDDSGLHGARPVPQESSRKNAKEALAVKAAKGDFVCIFCDRSFRKGKDYSKHLNRHLVNVYYLE

SEQ ID NO: 780 - protein, 80 aa, synthetic construct; identical to SEQ ID NO: 504

SEQ ID NO: 781 - protein, 80 aa, synthetic construct
APFKEEEKSPVSAPKLPKAQATSVIRHTADAQLCNHQTCPMKAASILNYQNNSFRRRTHLNVEAARKNIPCAAVSPNRSK

SEQ ID NO: 782 - protein, 80 aa, synthetic construct; identical to SEQ ID NO: 532

SEQ ID NO: 783 - protein, 80 aa, synthetic construct; identical to SEQ ID NO: 554

SEQ ID NO: 784 - protein, 80 aa, synthetic construct
GSGSESGKERRTTSKEISVIQHTSSFEKSDSLEQPSGLEGEDKPLAQFPSPPPAPHGRSAHSLQPKLVRQPNIQVPEILV

SEQ ID NO: 785 - protein, 80 aa, synthetic construct
SSAQKVQIKQETIESRLSELKSENESLWKEVSELRAKHAQQQQVIRKIVQFIVTLVQNNQLVSLKRKRPLLLNTNGAQKK

SEQ ID NO: 786 - protein, 80 aa, synthetic construct
TSPVLVPGCPSKAVDPGLPSVKQEPPDPEEDKEENKDDSASKLAPEEEAGGAGTPVITEIFSLGGTRFRDTAVWLPRSKD

SEQ ID NO: 787 - protein, 80 aa, synthetic construct
GQDSEQPQAQLQPEAQLHAPAQPQPQLQLQLQSHKGFLEQEGSPLSLGQSQHDLSQSAFLSWLSQTQSHSSLLSSSVLTP

SEQ ID NO: 788 - protein, 80 aa, synthetic construct
EIILCDTCPRAYHMVCLDPDMEKAPEGKWSCPHCEKEGIQWEAKEDNSEGEEILEEVGGDLEEEDDHHMEFCRVCKDGGE

SEQ ID NO: 789 - protein, 80 aa, synthetic construct
DSSGVIDLTMDDEESGASQDPKKLNHTPVSTMSSSQPVSRPLQPIQPAPPLQPSGVPTSGPSQTTIHLLPTAPTTVNVTH

SEQ ID NO: 790 - protein, 80 aa, synthetic construct
MHRTTRIKITELNPHLMCALCGGYFIDATTIVECLHSFCKTCIVRYLETNKYCPMCDVQVHKTRPLLSIRSDKTLQDIVY

SEQ ID NO: 791 - protein, 80 aa, synthetic construct
KIRQDSVTKLLTDVQLMKGKQECMDSKLLAMKHENEALWREVASLRQKHAQQQKVVNKLIQFLISLVQSNRILGVKRKIP

SEQ ID NO: 792 - protein, 80 aa, synthetic construct
LQQKLEEVRQPPPRVRPVPSLVPRPPQTSLVKFVGNIYTLKCRFCEVEFQGPLSIQEEWVRHLQRHILEMNFSKADPPPE

SEQ ID NO: 793 - protein, 80 aa, synthetic construct
MELSAIGEQVFAVESIRKKRVRKGKVEYLVKWKGWPPKYSTWEPEEHILDPRLVMAYEEKEERDRASGYRKRGPKPKRLL

SEQ ID NO: 794 - protein, 80 aa, synthetic construct
CGGRQDPDKQLMCDECDMAFHIYCLDPPLSSVPSEDEWYCPECRNDASEVVLAGERLRESKKKAKMASATSSSQRDWGKG

SEQ ID NO: 795 - protein, 80 aa, synthetic construct
ALSEVPQLKQEISECLIASSFEDDSRVASPLDQNGSFNVVIKEEPLDDYDYELGECPEGVTVKQEETDEETDVYSNSDDD

SEQ ID NO: 796 - protein, 80 aa, synthetic construct
SAGHEKLPVHVEDALTYLDQVKIRFGSDPATYNGFLEIMKEFKSQSIDTPGVIRRVSQLFHEHPDLIVGFNAFLPLGYRI

SEQ ID NO: 797 - protein, 80 aa, synthetic construct
TSTQVMVIKRPGRKRKAEADPQAIPKKRGRKPGSVVAAAAAEAKKKAVKESSIRSVQETVLPIKKRKTRETVSIEVKEVV

SEQ ID NO: 798 - protein, 80 aa, synthetic construct
MDSLEEPQKKVFKARKTMRVSDRQQLEAVYKVKEELLKTDVKLLNGNHENGDLDPTSPLENMDYIKDKEEVNGIEEICFD

SEQ ID NO: 799 - protein, 80 aa, synthetic construct
CHKKFRRQAYLRKHLSTHEAGSARALAPGFGSERGAPLAFACPLCGAHFPTADIREKHRLWHAVREELLLPALAGAPPET

SEQ ID NO: 800 - protein, 80 aa, synthetic construct
LLDSSNSSASASQAMNIKIEPEETTEARTHNLRRRMGCPTPKCENEKEMKSSIKQEPIERKDYIVENEKEKPKSRSKKAT

SEQ ID NO: 801 - protein, 80 aa, synthetic construct
PVSGSSPFATQVGVIRCPVCSQECAERHIIDNFFVKDTTEVPSSTVEKSNQVCTSCEDNAEANGFCVECVEWLCKTCIRA

SEQ ID NO: 802 - protein, 80 aa, synthetic construct
MAEDWLDCPALGPGWKRREVFRKSGATCGRSDTYYQSPTGDRIRSKVELTRYLGPACDLTLFDFKQGILCYPAPKAHPVA

SEQ ID NO: 803 - protein, 80 aa, synthetic construct
PVQGQQQFQRLKVEDALSYLDQVKLQFGSQPQVYNDFLDIMKEFKSQSIDTPGVISRVSQLFKGHPDLIMGFNTFLPPGY

SEQ ID NO: 804 - protein, 80 aa, synthetic construct
RAKQPDLAPGLTTIGASPTQTVTLVTQPVVTKETAISKLEMPSSLMLEVPALADFNRAWTELTDWLSLLDQVIKSQRVMV

SEQ ID NO: 805 - protein, 80 aa, synthetic construct
HMRCHQHFLRTEAKVKEEIPDPDVKGSPHLSDSACLGQQREGGGTELVGTMMTSNTPERTSQGGAGVSPLLVKEEPKEDN

SEQ ID NO: 806 - protein, 80 aa, synthetic construct
QRQTIVIPDTPSPTVSVITISSDTDEEEEQKHAPTSTVSKQRKNVISCVTVHDSPYSDSSSNTSPYSVQQRAGHNNANAF

SEQ ID NO: 807 - protein, 80 aa, synthetic construct
SGDGTQRQRLKTLCKDCRAQRIAFNREQRMFKRVGCGECAACQVTEDCGACSTCLLQLPHDVASGLFCKCERRRCLRIVE

SEQ ID NO: 808 - protein, 80 aa, synthetic construct
KMAEKAREKEEECEKYGPDYVPQKSHGKKVKREPAEPHETSEPTYLRFIKSLAERTMSVTTDSTVTTSPYAFTRVTGPYN

SEQ ID NO: 809 - protein, 80 aa, synthetic construct
GSGPPSSGGGLYRDMGAQGGRPSLIARIPVARILGDPEEESWSPSLTNLEKVVVTDVTSNFLTVTIKESNTDQGFFKEKR

SEQ ID NO: 810 - protein, 80 aa, synthetic construct
GSTMDSAGLGQEAPALFRPCTPAVWAPAPAAGGPRSPPPLLLLPESLPGSSASVPPPQPASSRCAESPGLGLRVWRPWGS

SEQ ID NO: 811 - protein, 80 aa, synthetic construct
EDGHAISTIKEEPKIDFRVYNLLTPDSKMGEPVSESVVKREKLEEKDGLKEKVWTESSSDDLRNVTWRGADILRGSPSYT

SEQ ID NO: 812 - protein, 80 aa, synthetic construct
VGSRGSSGSSSKPAGADSTHKVPVVMLEPIRIKQENSGPPENYDFPVVIVKQESDEESRPQNANYPRSILTSLLLNSSQS

SEQ ID NO: 813 - protein, 80 aa, synthetic construct
NEGDQPKTPENIPSKEPKDGSPVQPSLLSLMKDRRLTLEQVVAIEALTQLSEAPSENSSPSKSEKDEESEQRTASLLNSC

SEQ ID NO: 814 - protein, 80 aa, synthetic construct
CRKGDNEELLLLCDGCDKGCHTYCHRPKITTIPDGDWFCPACIAKASGQTLKIKKLHVKGKKTNESKKGKKVTLTGDTED

SEQ ID NO: 815 - protein, 80 aa, synthetic construct
SDEQKIKPVTENLVLSSHTGFCQSSGDEALSKSVPVTVDDDDDDNDPENRIAKKMLLEEIKANLSSDEDGSSDDEPEEGK

SEQ ID NO: 816 - protein, 80 aa, synthetic construct
QQHKVLQEDLEQEQVRVNSLTHMVVVVDESSGDHATAALEEQLKVLGDRWANICRWTEDRWVLLQDILLKWQRLTEEQCL

SEQ ID NO: 817 - protein, 80 aa, synthetic construct
QVCTSCEDNAEANGFCVECVEWLCKTCIRAHQRVKFTKDHTVRQKEEVSPEAVGVTSQRPVFCPFHKKEQLKLYCETCDK

SEQ ID NO: 818 - protein, 80 aa, synthetic construct
LDVSIKMEKDSGCEGAADGCVPSQVWLGASDRSHPATFPTRMHLKTEPDSRQQVYISHLGHGVRGAQPHGRATAGRSREL

SEQ ID NO: 819 - protein, 80 aa, synthetic construct
MELPAVGEHVFAVESIEKKRIRKGRVEYLVKWRGWSPKYNTWEPEENILDPRLLIAFQNRERQEQLMGYRKRGPKPKPLV

SEQ ID NO: 820 - protein, 80 aa, synthetic construct
GSLQGAFQPKTSNPIDVAARPGTVPHTLLQKDPRLTDPFRPMLRKPGKWCAMHVHIAWQIYHHQQKVKKQMQSDPHKLDF

SEQ ID NO: 821 - protein, 80 aa, synthetic construct
LKVKTEPVDFLDPFTCNVSVPIKDELLPVMSLPSSELLSKPFTNTLQLNLYNTPFQSMQSSGSAHQMITTLPLGMTCPID

SEQ ID NO: 822 - protein, 80 aa, synthetic construct
LVKEEPKEDNGLPTSFTLNAADRPANHTKLKDPSEYVANSASALFSQDISVKMASDFLMKLSAANQKEPMNLNFKVKEEP

SEQ ID NO: 823 - protein, 80 aa, synthetic construct
EPQNHFSSFKYSGNAVVESYSVLGNCRPSDPYSMNSVYSYHSYYAQPSLTSVNGFHSKYALPSFSYYGFPSSNPVFPSQF

SEQ ID NO: 824 - protein, 80 aa, synthetic construct
IPEFDPGATKYTDKDNGGMLVWSPYHSIPDAKLDEYIAIAKEKHGYNVEQALGMLFWHKHNIEKSLADLPNFTPFPDEWT

SEQ ID NO: 825 - protein, 80 aa, synthetic construct
KNPVSFHPLFEGGLCQTCRDRFLELFYMYDDDGYQSYCTVCCEGRELLLCSNTSCCRCFCVECLEVLVGTGTAAEAKLQE

SEQ ID NO: 826 - protein, 80 aa, synthetic construct
DTTKAPKGSLKDIYKVFNGEGEQIRAFKCEHCRVLFLDHVMYTIHMGCHGYRDPLECNICGYRSQDRYEFSSHIVRGEHT

SEQ ID NO: 827 - protein, 80 aa, synthetic construct
PAVRTRNNNSVSSRERHRPSPRSTRGRQGRNHVDESPVEFPATRSLRRRATASAGTPWPSPPSSYLTIDLTDDTEDTHGT

SEQ ID NO: 828 - protein, 80 aa, synthetic construct
AESYKETQMVKIKEEPMEVDIQDSHVSISPSRNVGYSTLIGREKTEPLQKMPEGRVPPERNLFSQDISVKMASELLFQLS

SEQ ID NO: 829 - protein, 80 aa, synthetic construct
NQKLQKVIQWLLEEKLCALQCAVFDKTLAELKTRVEKIECNKRHKTVLTELQAKIARLTKRFEAAKEDLKKRHEHPPNPP

SEQ ID NO: 830 - protein, 80 aa, synthetic construct
VVVKEELGGDVKVESTSPKTFLESKEELSHSPEPCTKMTMRLRRNHSNAQFIESYVCRMCSRGDEDDKLLLCDGCDDNYH

SEQ ID NO: 831 - protein, 80 aa, synthetic construct
MAENLKGCSVCCKSSWNQLQDLCRLAKLSCPALGISKRNLYDFEVEYLCDYKKIREQEYYLVKWRGYPDSESTWEPRQNL

SEQ ID NO: 832 - protein, 80 aa, synthetic construct
APCKLGSQAGEAAKVFGGFQVVPAPDGQFAFLIPNGAFAHSGPVIPVYTSNSGTSVGPNAVSPSSGPSLTADSMWRPWRN

SEQ ID NO: 833 - protein, 80 aa, synthetic construct
VKKEDSKEQPDLPSLAPPSSLPLSETSSRPAKSQEGTDSKKVLQFPSLHTTTNVSWCYLNYIKPNHIQHADRRSSVYAGW

SEQ ID NO: 834 - protein, 80 aa, synthetic construct
QLSCSVKEEPNVDGQEVAPSSPPLAAQSPEGNHGHQEPASTSFHPPRIQEEWGLLDRSQKELYWDAMLEKYGTVVSLGLP

SEQ ID NO: 835 - protein, 80 aa, synthetic construct
TSSVIRLNSPTTTSQIMARKKRRGIIEKRRRDRINNSLSELRRLVPTAFEKQGSAKLEKAEILQMTVDHLKMLQATGGKG

SEQ ID NO: 836 - protein, 80 aa, synthetic construct
TVVKTEVCSPLRDQEYGQPCSRRPDSSAMEVEPKKLKGKRDLIVPKSFQQVDFWFCESCQEYFVDECPNHGPPVFVSDTP

SEQ ID NO: 837 - protein, 80 aa, synthetic construct
EGQYVLCRWTDGLYYLGKIKRVSSSKQSCLVTFEDNSKYWVLWKDIQHAGVPGEEPKCNICLGKTSGPLNEILICGKCGL

SEQ ID NO: 838 - protein, 80 aa, synthetic construct
KVEEAEPEEFVVEKVLDRRVVNGKVEYFLKWKGFTDADNTWEPEENLDCPELIEAFLNSQKAGKEKDGTKRKSLSDSESD

SEQ ID NO: 839 - protein, 80 aa, synthetic construct
NPELPPRLGPVPSGLSQKGTQIPDHFRPPLRKPGKWCAMHVRVAYMILRHQEKMKGDSHKLDFRNDLLPCLPGPYGALPP

SEQ ID NO: 840 - protein, 80 aa, synthetic construct
FPSKVCLQLKIREVRQKIMQAATPTEQPPGAEAPLPVPPPTGTAAAPAPTPSPAGGPDPTSPSSDSGTAQAAPPLPPPPE

SEQ ID NO: 841 - protein, 80 aa, synthetic construct
EEEEEEEEEDMSHFSLRLEGGRQDSEDEEERLINLSELTPYILCSICKGYLIDATTITECLHTFCKSCIVRHFYYSNRCP

SEQ ID NO: 842 - protein, 80 aa, synthetic construct
DDKKTSNLSVLRTALRYIQSLKRKEKEYEHEMERLAREKIATQQRLAELKHELSQWMDVLEIDRVLRQTGQPEDDQASTS

SEQ ID NO: 843 - protein, 80 aa, synthetic construct
LEHVHGSGPYSAPSPAYSSSSLYAPDAVASSGPIISDITELAPASPMASPGGSIDERPLSSSPLVRVKEEPPSPPQSPRV

SEQ ID NO: 844 - protein, 80 aa, synthetic construct
ITTSTGFLTDLTLDDILFADIDTSMYDFDPCTSSSGTASKMAPVSADDLLKTLAPYSSQPVTPSQPFKMDLTELDHIMEV

SEQ ID NO: 845 - protein, 80 aa, synthetic construct
MHRTTRIKITELNPHLMCVLCGGYFIDATTIIECLHSFCKTCIVRYLETSKYCPICDVQVHKTRPLLNIRSDKTLQDIVY

SEQ ID NO: 846 - protein, 80 aa, synthetic construct
RPRLPGPLPPGEAGLPAPSTRPLLSSEVPQISPQEGLPLSQSALSIAKEKNISLQTAIAIEALTQLSSALPQPSHSTPQA

SEQ ID NO: 847 - protein, 80 aa, synthetic construct
MYDPKTRSLNYCGHIYTPISCKIRDLLPVMCDRAGFIQDTSLILYEEVKPNLTERIQDYDVSLDKALDELMDGDIIVFQK

SEQ ID NO: 848 - protein, 80 aa, synthetic construct
SQPPVFKVFPGSTTEDYNLIVIERGAAAAATGQPGTAPAGTPGAPPLAGMAIVKEEETEAAIGAPPTATEGPETKPVLMA

SEQ ID NO: 849 - protein, 80 aa, synthetic construct
TVAGIPASKVSKWSTDEVSEFIQSLPGCEEHGKVFKDEQIDGEAFLLMTQTDIVKIMSIKLGPALKIFNSILMFKAAEKN

SEQ ID NO: 850 - protein, 80 aa, synthetic construct
PQEGLLRGTPGPSKEVLRVVGESGEPVKAFKCEHCRILFLDHVMFTIHMGCHGFRDPFECNICGYHSQDRYEFSSHIVRG

SEQ ID NO: 851 - protein, 80 aa, synthetic construct
CGSGNDEDRLLLCDGCDDSYHTFCLIPPLHDVPKGDWRCPKCLAQECSKPQEAFGFEQAARDYTLRTFGEMADAFKSDYF

SEQ ID NO: 852 - protein, 80 aa, synthetic construct
QVVMPPLVRGAQQIHSIRQHSSTGPPPLLLAPRASVPSVQIQGQRIIQQGLIRVANVPNTSLLVNIPQPTPASLKGTTAT

SEQ ID NO: 853 - protein, 80 aa, synthetic construct
QERIQRLEEDRQSLDLSSEWWDDKLHARGSSRSWDSLPPSKRKKAPLVSGPYIVYMLQEIDILEDWTAIKKARAAVSPQK

SEQ ID NO: 854 - protein, 80 aa, synthetic construct
PLSKPRTPRRSKSDGEAKPEPSPSPRITRKSTRQTTITSHFAKGPAKRKPQEESERAKSDESIKEEDKDQDEKRRRVTSR

SEQ ID NO: 855 - protein, 80 aa, synthetic construct
MGHMHLHSDSKPFKCLYCPSKFTLKGNLTRHMKVKHGVMERGLHSQGLGRGRIALAQTAGVLRSLEQEEPFDLSQKRRAK

SEQ ID NO: 856 - protein, 80 aa, synthetic construct
GELQLYRVLQRANLLSYYETFIQQGGDDVQQLCEAGEEEFLEIMALVGMATKPLHVRRLQKALREWATNPGLFSQPVPAV

SEQ ID NO: 857 - protein, 80 aa, synthetic construct
QLQEKHVKDEQIEHWKKIVKTQEELKELLNKMVNLKEKIKELHQQYKEASEVKPPRDITAEFLVKSKHRDLTALCKEYDE

SEQ ID NO: 858 - protein, 80 aa, synthetic construct
LQSKKDTETENPPVENTLSPKLEITEKWEKYWNEYGGGLLWQSWQEKHPGQALSSEPWNFPDTKEEWEQHYSQLYWYYLE

SEQ ID NO: 859 - protein, 80 aa, synthetic construct
VSKEPGQLEDEEEEPSSFKADSPAEASLASDPHELPTTSFCPNCIRLKKKVRELQAELDMLKSGKLPEPPVLPPQVLELP

SEQ ID NO: 860 - protein, 80 aa, synthetic construct
PGEENLLGEAAGGQDMADSMLMQGSRGLTDQAIFYAVTPLPWCPHLVAVCPIPAAGLDVTQPCGDCGTIQENWVCLSCYQ

SEQ ID NO: 861 - protein, 80 aa, synthetic construct
MGKKQNKKKVEEVLEEEEEEYVVEKVLDRRVVKGKVEYLLKWKGFSDEDNTWEPEENLDCPDLIAEFLQSQKTAHETDKS

SEQ ID NO: 862 - protein, 80 aa, synthetic construct
VEEKVNQLKEEVRLQYEKLHQLLDEDLRQTVEVLDKAQAKFCSENAAQALHLGERMQEAKKLLGSLQLLFDKTEDVSFMK

SEQ ID NO: 863 - protein, 80 aa, synthetic construct
QTHPSLASQGSKKSKSSSKSTTSQIPLQAQEDCCVHCILSCLFCEFLTLCNIVLDCATCGSCSSEDSCLCCCCCGSGECA

SEQ ID NO: 864 - protein, 80 aa, synthetic construct
HRGSTAGEVYRSHLPTHLDPAMPFHRALDPAAAAYLFQRQLSPTPGYPSQYQLYAMENTRQTILNDYITSQQMQVNLRPD

SEQ ID NO: 865 - protein, 80 aa, synthetic construct
KKERKPQKPGKYICQYCSRPCAKPSVLQKHIRSHTGERPYPCGPCGFSFKTKSNLYKHRKSHAHRIKAGLASGMGGEMYP

SEQ ID NO: 866 - protein, 80 aa, synthetic construct
AASENSQDALRVVSTSGEQMKVYKCEHCRVLFLDHVMYTIHMGCHGFRDPFECNMCGYHSQDRYEFSSHITRGEHRFHMS

SEQ ID NO: 867 - protein, 80 aa, synthetic construct
TSSQYRQLLSDYGPPSLGYTQGTGNSQVPQSKYAELLAIIEELGKEIRPTYAGSKSAMERLKRGIIHARGLVRECLAETE

SEQ ID NO: 868 - protein, 80 aa, synthetic construct
SEGESEEVEVTDISDEDEEDGEVFKTPRAPPAPPKPEPGEAPGASQCMPLKLRFKRRWSEDCRLEGGGGPAGGFEDEGED

SEQ ID NO: 869 - protein, 80 aa, synthetic construct
IPKGVWRCPKCVMAECKRPPEAFGFEQATREYTLQSFGEMADSFKADYFNMPVHMVPTELVEKEFWRLVNSIEEDVTVEY

SEQ ID NO: 870 - protein, 80 aa, synthetic construct
LLGWITAHVLKQEVLPAAQKTEEPLGSPHPSGTVESPGEGPQDTRIEGSVQLSCSVKEEPNVDGQEVAPSSPPLAAQSPE

SEQ ID NO: 871 - protein, 80 aa, synthetic construct
DPRELRERHRPLVVPGSRQEEVIDHKLTEREWAEEWKHLNNLLNCIMDMVEKTRRSLTVLRRCQEADREELNHWARRYSD

SEQ ID NO: 872 - protein, 80 aa, synthetic construct
SDRREDYYDIQLSIKGKKNIFESFVDYVAVEQLDGDNKYDAGEHGLQEAEKGVKFLTLPPVLHLQLMRFMYDPQTDQNIK

SEQ ID NO: 873 - protein, 80 aa, synthetic construct
EIAELQQAVVEELGISMEELRHFIDEELEKMDCVQQRKKQLAELETWVIQKESEVAHVDQLFDDASRAVTNCESLVKDFY

SEQ ID NO: 874 - protein, 80 aa, synthetic construct
FPTDIPKSAPHSMTASSSSVSSPSPGLPRRSAPPSPLCRSLSPGTGGGVRGGVGYLSRGDPVRVLARRVRPDGSVQYLVE

SEQ ID NO: 875 - protein, 80 aa, synthetic construct
LKPPPICPRDSVKVINKEGEVMDVYRCDHCRVLFLDYVMFTIHMGCHGFRDPFECNMCGYRSHDRYEFSSHIARGEHRAL

SEQ ID NO: 876 - protein, 80 aa, synthetic construct
EHQALLDEPYLDRLPGQKEAHAQAGVQVKQEPIESDEEEAEPPREVEPGQRQPSEQELLFRQQALLLEQQRIHQLRNYQA

SEQ ID NO: 877 - protein, 80 aa, synthetic construct
AEPSMDVILVGSSELSSSVSPGTGRDLIAYEVKANQRNIEDICICCGSLQVHTQHPLFEGGICAPCKDKFLDALFLYDDD

SEQ ID NO: 878 - protein, 80 aa, synthetic construct
SSACVDDTLGQVGAVKVKEEPVDSDEDAQIQEMESGEQAAFMQQPFLEPTHTRALSVRQAPLAAVGMDGLEKHRLVSRTH

SEQ ID NO: 879 - protein, 80 aa, synthetic construct
VKVEEEEEASLSQGGESSHDHIAHSEAARLRFRHFRYEEASGPHEALAHLRALCCQWLQPEAHSKEQILELLVLEQFLGA

SEQ ID NO: 880 - protein, 80 aa, synthetic construct
QDIHSIQAFHEQIVIAVKAPAETRLDVPAPREDSITVHIRSTNGPIDVYLCEVEQGQTSNKRSEGVGTSSSESTHPEGPE

SEQ ID NO: 881 - protein, 80 aa, synthetic construct
AAQNCQRPCKDKVDWVQCDGGCDEWFHQVCVGVSPEMAENEDYICINCAKKQGPVSPGPAPPPSFIMSYKLPMEDLKETS

SEQ ID NO: 882 - protein, 80 aa, synthetic construct
KEGKIVDAKAIFTGHSAVVEDVAWHLLHESLFGSVADDQKLMIWDTRSNTTSKPSHLVDAHTAEVNCLSFNPYSEFILAT

SEQ ID NO: 883 - protein, 80 aa, synthetic construct
EREREKLREVIEIEDASPTKCPITTKLVLDEDEETKEPLVQVHRNMVIKLKPHQVDGVQFMWDCCCESVKKTKKSPGSGC

SEQ ID NO: 884 - protein, 80 aa, synthetic construct
EPETARLRFRGFCYQEVAGPREALARLRELCCQWLQPEAHSKEQMLEMLVLEQFLGTLPPEIQAWVRGQRPGSPEEAAAL

SEQ ID NO: 885 - protein, 80 aa, synthetic construct
PVVQNAASIVQPSPAHVGQQGLSKLPSRPGAQGVEPQNLRTLQGHSVIRSATNTTLPHMLMSQRVIAPNPAQLQGQRGPP

SEQ ID NO: 886 - protein, 80 aa, synthetic construct
TESCHSQMHRPIKVEPGCKPHACMHTAPPENKTWKKVTKQENPPASCDNVQQKSIIETMEQHLKQFHAKSLFDHKALTLK

SEQ ID NO: 887 - protein, 80 aa, synthetic construct
VVPKEEKEEETAAAPPVEEGEEQKPPAAEELAVDTGKAEEPVKSECTEEAEEGPAKGKDAEAAEATAEGALKAEKKEGGS

SEQ ID NO: 888 - skipped

SEQ ID NO: 889 - protein, 80 aa, synthetic construct
PPMKSLSSTSAGGKKQAQPSCAPASRPPAKQQKIKENQKTDVLCADEEEDCQAASLLQKYTDNSEKPSGKRLCKTKHLIP

SEQ ID NO: 890 - protein, 80 aa, synthetic construct
TSRDSSPVMRSSSTLPVPQPSSAPPTPTRLTGANSDMEEEERGDLIQFYNNIYIKQIKTFAMKYSQANMDAPPLSPYPFV

SEQ ID NO: 891 - protein, 80 aa, synthetic construct
IPRGIWRCPKCILAECKQPPEAFGFEQATQEYSLQSFGEMADSFKSDYFNMPVHMVPTELVEKEFWRLVSSIEEDVTVEY

SEQ ID NO: 892 - protein, 80 aa, synthetic construct
STQHLVALPGGAQPVHSSPVFPPSQYPNGSAAQQPMLPQYGGRKILVCSVDNCYCSSVANHGGHQPYPRSGHFPWTVPSQ

SEQ ID NO: 893 - protein, 80 aa, synthetic construct
ILPVIQPVVVQPVPFMYTSHLQQPLMVSLSEEMENSSSSMQVPVIESYEKPISQKKIKIEPGIEPQRTDYYPEEMSPPLM

SEQ ID NO: 894 - protein, 80 aa, synthetic construct
YGGLKTPTQLEGATRGTTEPHSRGHLSRPEAQSLSPYTTSANRAKLLAKNRQTFLLQTTKLTRLARRMCRDLLQPRRAAR

SEQ ID NO: 895 - protein, 80 aa, synthetic construct
MAQPPRLSRSGASSLWDPASPAPTSGPRPRLWEGQDVLARWTDGLLYLGTIKKVDSAREVCLVQFEDDSQFLVLWKDISP

SEQ ID NO: 896 - protein, 80 aa, synthetic construct
AKLARRSQERDNLGMLVWSPNQNLSEAKLDEYIAIAKEKHGYNMEQALGMLFWHKHNIEKSLADLPNFTPFPDEWTVEDK

SEQ ID NO: 897 - DNA, 240 bp, synthetic construct
  source 1..240: mol_type = other DNA
aagatgaagaagttccagatccccgtgagcttccaggacctgaccgtgaacttcacccaggaggagtggcagcagctggaccccgcccagagactgctgtacagagatgtgatgctggagaactacagcaacctggtgagcgtgggctaccacgtgagcaagcccgacgtgatcttcaagctggagcagggcgaggagccctggatcgtggaggagttcagcaaccagaactaccccgac

SEQ ID NO: 898 - DNA, 240 bp, synthetic construct
  source 1..240: mol_type = other DNA
tgcagccaggagagcgccctgagcgaggaggaggaggacaccaccagacccctggagaccgtgaccttcaaggacgtggccgtggacctgacccaggaggagtgggagcagatgaagcccgcccagagaaacctgtacagagatgtgatgctggagaactacagcaacctggtgaccgtgggctgccaggtgaccaagcccgacgtgatcttcaagctggagcaggaggaggagccctgg

SEQ ID NO: 899 - DNA, 240 bp, synthetic construct
  source 1..240: mol_type = other DNA
atcaccagccagggcagcgtgagcttcagagatgtgaccgtgggcttcacccaggaggagtggcagcacctggaccccgcccagagaaccctgtacagagatgtgatgctggagaactacagccacctggtgagcgtgggctactgcatccccaagcccgaggtgatcctgaagctggagaagggcgaggagccctggatcctggaggagaagttccccagccagagccacctggagctg

SEQ ID NO: 900 - DNA, 240 bp, synthetic construct
  source 1..240: mol_type = other DNA
ccccaggtgaccttcaacgacgtggccatcgacttcacccacgaggagtggggctggctgagcagcgcccagagagacctgtacaaggacgtgatggtgcagaactacgagaacctggtgagcgtggccggcctgagcgtgaccaagccctacgtgatcaccctgctggaggacggcaaggagccctggatgatggagaagaagctgagcaagggcatgatccccgactgggagagcaga

SEQ ID NO: 901 - DNA, 240 bp, synthetic construct
  source 1..240: mol_type = other DNA
cccctgagattcagcaccctgttccaggagcagcagaagatgaacatcagccaggccagcgtgagcttcaaggacgtgaccatcgagttcacccaggaggagtggcagcagatggcccccgtgcagaagaacctgtacagagatgtgatgctggagaactacagcaacctggtgagcgtgggctactgctgcttcaagcccgaggtgatcttcaagctggagcagggcgaggagccctgg

SEQ ID NO: 902 - DNA, 240 bp, synthetic construct
  source 1..240: mol_type = other DNA
caggaccccagcgccgagggcctgagcgaggaggtgcccgtggtgttcgaggagctgcccgtggtgttcgaggacgtggccgtgtacttcaccagagaggagtggggcatgctggacaagagacagaaggagctgtacagagatgtgatgagaatgaactacgagctgctggccagcctgggccccgccgccgccaagcctgacctgatcagcaagctggagagaagagccgccccctgg

SEQ ID NO: 903 - DNA, 240 bp, synthetic construct
  source 1..240: mol_type = other DNA
agccagggcagcgtgaccttcagagatgtggccatcgacttcagccaggaggagtggaagtggctgcagcccgcccagagagacctgtacagatgcgtgatgctggagaactacggccacctggtgagcctgggcctgagcatcagcaagcccgacgtggtgagcctgctggagcagggcaaggagccctggctgggcaagagagaggtgaagagagacctgttcagcgtgagcgagagc

SEQ ID NO: 904 - DNA, 240 bp, synthetic construct
  source 1..240: mol_type = other DNA
ggcagcctgaccttcagagatgtggccatcgagttcagcctggaggagtggcagtgcctggacaccgcccagcagaacctgtacagaaacgtgatgctggagaactacagaaacctggtgttcctgggcatcgccgccttcaagcccgacctgatcatcttcctggaggagggcaaggagagctggaacatgaagagacacgagatggtggaggagagccccgtgatctgcagccacttc

SEQ ID NO: 905 - DNA, 240 bp, synthetic construct
  source 1..240: mol_type = other DNA
aacaagagccaggagcaggtgagcttcaaggacgtgtgcgtggacttcacccaggaggagtggtacctgctggaccccgcccagaagatcctgtacagagatgtgatcctggagaactacagcaacctggtgagcgtgggctactgcatcaccaagcccgaggtgatcttcaagatcgagcagggcgaggagccctggatcctggagaagggcttccccagccagtgccaccccgagaga

SEQ ID NO: 906 - DNA, 240 bp, synthetic construct
  source 1..240: mol_type = other DNA
ccccacctgctggtgaccttcagagatgtggccatcgacttcagccaggaggagtgggagtgcctggaccccgcccagagagacctgtacagagatgtgatgctggagaactacagcaacctgatcagcctggacctggagagcagctgcgtgaccaagaagctgagccccgagaaggagatctacgagatggagagcctgcagtgggagaacatgggcaagagaatcaaccaccacctg

SEQ ID NO: 907 - DNA, 240 bp, synthetic construct
  source 1..240: mol_type = other DNA
gaggaggagagaaagaccgccgagctgcagaagaacagaatccaggacagcgtggtgttcgaggacgtggccgtggacttcacccaggaggagtgggccctgctggacctggcccagagaaacctgtacagagatgtgatgctggagaacttccagaacctggccagcctgggctaccccctgcacaccccccacctgatcagccagtgggagcaggaggaggacctgcagaccgtgaag

SEQ ID NO: 908 - DNA, 240 bp, synthetic construct
  source 1..240: mol_type = other DNA
ggcctgctgaccttcagagatgtggccatcgagttcagcctggaggagtggcagtgcctggacaccgcccagaagaacctgtacagaaacgtgatgctggagaactacagaaacctggccttcctgggcatcgccgtgagcaagcccgacctgatcatctgcctggagaaggagaaggagccctggaacatgaagagagatgagatggtggacgagccccccggcatctgcccccacttc

SEQ ID NO: 909 - DNA, 240 bp, synthetic construct
  source 1..240: mol_type = other DNA
agagcccccacccaggtgaccgtgagccccgagacccacatggacctgaccaagggctgcgtgaccttcgaggacatcgccatctacttcagccaggacgagtggggcctgctggacgaggcccagagactgctgtacctggaggtgatgctggagaacttcgccctggtggccagcctgggctgcggccacggcaccgaggacgaagagacccctagcgaccagaacgtgagcgtgggc

SEQ ID NO: 910 - DNA, 240 bp, synthetic construct
  source 1..240: mol_type = other DNA
gccgccgtgagccccaccaccagatgccaggagagcgtgaccttcgaggacgtggccgtggtgttcaccgacgaggagtggagcagactggtgcccatccagagagacctgtacaaggaggtgatgctggagaactacaacagcatcgtgagcctgggcctgcccgtgccccagcccgacgtgatcttccagctgaagagaggcgacaagccctggatggtggacctgcacggcagcgag

SEQ ID NO: 911 - DNA, 240 bp, synthetic construct
  source 1..240: mol_type = other DNA
cacagcctggagaaggtgaccttcgaggacgtggccatcgacttcacccaggaggagtgggacatgatggacaccagcaagagaaagctgtacagagatgtgatgctggagaacatcagccacctggtgagcctgggctaccagatcagcaagagctacatcatcctgcagctggagcagggcaaggagctgtggagagagggcagagtgttcctgcaggaccagaaccccgacagagag

SEQ ID NO: 912 - DNA, 240 bp, synthetic construct
  source 1..240: mol_type = other DNA
gacagcgtgagcttcgaggacgtggccgtgaacttcaccctggaggagtgggccctgctggacagcagccagaagaagctgtacgaggacgtgatgcaggagaccttcaagaacctggtgtgcctgggcaagaagtgggaggaccaggacatcgaggacgaccacagaaaccagggcaagaacagaagatgccacatggtggagagactgtgcgagagcagaagaggcagcaagtgcggc

SEQ ID NO: 913 - DNA, 240 bp, synthetic construct
  source 1..240: mol_type = other DNA
aacgtggaggtggtgaaggtgatgccccaggacctggtgaccttcaaggacgtggccatcgacttcagccaggaggagtggcagtggatgaaccccgcccagaagagactgtacagaagcatgatgctggagaactaccagagcctggtgagcctgggcctgtgcatcagcaagccctacgtgatcagcctgctggagcagggcagagagccctgggagatgaccagcgagatgaccaga

SEQ ID NO: 914 - DNA, 240 bp, synthetic construct
  source 1..240: mol_type = other DNA
acccagcccgacgaggacctgcacctgcaggccgaggagacccagctggtgaaggagagcgtgaccttcaaggacgtggccatcgacttcaccctggaggagtggagactgatggaccccacccagagaaacctgcacaaggacgtgatgctggagaactacagaaacctggtgagcctgggcctggccgtgagcaagcccgacatgatcagccacctggagaacggcaagggcccctgg

SEQ ID NO: 915 - DNA, 240 bp, synthetic construct
  source 1..240: mol_type = other DNA
accatgctgcaggagagcttcagcttcgacgacctgagcgtggacttcacccagaaggagtggcagctgctggaccccagccagaagaacctgtacaaggacgtgatgctggagaactacagcagcctggtgagcctgggctacgaggtgatgaagcccgacgtgatcttcaagctggagcagggcgaggagccctgggtgggcgacggcgagatccctagcagcgacagccccgaggtg

SEQ ID NO: 916 - DNA, 240 bp, synthetic construct
  source 1..240: mol_type = other DNA
gaggtggtgaccttcggcgacgtggccgtgcacttcagcagagaggagtggcagtgcctggaccccggccagagagccctgtacagagaggtgatgctggagaaccacagcagcgtggccggcctggccggcttcctggtgttcaagcccgagctgatcagcagactggagcagggcgaggagccctgggtgctggacctgcagggcgccgaaggcacagaggcccccagaaccagcaag

SEQ ID NO: 917 - DNA, 240 bp, synthetic construct
  source 1..240: mol_type = other DNA
agaaacgccgaggaggagagaatgatcgccgtgttcctgaccacctggctgcaggagcccatgaccttcaaggacgtggccgtggagttcacccaggaggagtggatgatgctggacagcgcccagagaagcctgtacagagatgtgatgctggagaactacagaaacctgaccagcgtggagtaccagctgtacagactgaccgtgatcagccccctggaccaggaggagatcagaaac

SEQ ID NO: 918 - DNA, 240 bp, synthetic construct
  source 1..240: mol_type = other DNA
ggcccccagggcgccagaagacaggccttcctggccttcggcgacgtgaccgtggacttcacccagaaggagtggagactgctgagccccgcccagagagccctgtacagagaggtgaccctggagaactacagccacctggtgagcctgggcatcctgcacagcaagcccgagctgatcagaagactggagcagggcgaggtgccctggggcgaggagagaagaagaagacccggcccc

SEQ ID NO: 919 - DNA, 240 bp, synthetic construct
  source 1..240: mol_type = other DNA
cacagcctgaagaagctgaccttcgaggacgtggccatcgacttcacccaggaggagtgggccatgatggacaccagcaagagaaagctgtacagagatgtgatgctggagaacatcagccacctggtgagcctgggctaccagatcagcaagagctacatcatcctgcagctggagcagggcaaggagctgtggagagagggcagagtgttcctgcaggaccagaaccccaacagagag

SEQ ID NO: 920 - DNA, 240 bp, synthetic construct
  source 1..240: mol_type = other DNA
atgcccgaggtggagttccccgaccagttcttcaccgtgctgaccatggaccacgagctggtgaccctgagagatgtggtgatcaacttcagccaggaggagtgggagtacctggacagcgcccagagaaacctgtactgggacgtgatgatggagaactacagcaacctgctgagcctggacctggagagcagaaacgagaccaagcacctgagcgtgggcaaggacatcatccagaac

SEQ ID NO: 921 - DNA, 240 bp, synthetic construct
  source 1..240: mol_type = other DNA
cccggcgcccccggcagcctggagatgggccccctgaccttcagagatgtgaccatcgagttcagcctggaggagtggcagtgcctggacaccgtgcagcagaacctgtacagagatgtgatgctggagaactacagaaacctggtgttcctgggcatggccgtgttcaagcccgacctgatcacctgcctgaagcagggcaaggagccctggaacatgaagagacacgagatggtgacc

SEQ ID NO: 922 - DNA, 240 bp, synthetic construct
  source 1..240: mol_type = other DNA
agagaccccgcccaggtgcccgtggccgccgacctgctgaccgaccacgaagaaggctacgtgaccttcgaggacgtggccgtgtacttcagccaggaggagtggagactgctggacgacgcccagagactgctgtacagaaacgtgatgctggagaacttcaccctgctggccagcctgggcctggccagcagcaagacccacgagatcacccagctggagagctgggaggagcccttc

SEQ ID NO: 923 - DNA, 240 bp, synthetic construct
  source 1..240: mol_type = other DNA
cacagcctgaagaaggtgaccttcgaggacgtggccatcgacttcacccaggaggagtgggccatgatggacaccagcaagagaaagctgtacagagatgtgatgctggagaacatcagccacctggtgagcctgggctaccagatcagcaagagctacatcatcctgcagctggagcagggcaaggagctgtggagagagggcagagagttcctgcaggaccagaaccccgacagagag

SEQ ID NO: 924 - DNA, 240 bp, synthetic construct
  source 1..240: mol_type = other DNA
accaagagcaaggaggccgtgaccttcaaggacgtggccgtggtgttcagcgaggaggagctgcagctgctggacctggcccagagaaagctgtacagagatgtgatgctggagaacttcagaaacgtggtgagcgtgggccaccagagcacccccgacggcctgccccagcttgagagagaggagaagctgtggatgatgaagatggccacccagagagacaacagcagcggcgccaag

SEQ ID NO: 925 - DNA, 240 bp, synthetic construct
  source 1..240: mol_type = other DNA
agccaggtgaccttcagcgacgtggccatcgacttcagccacgaggagtgggcctgcctggacagcgcccagagagacctgtacaaggacgtgatggtgcagaactacgagaacctggtgagcgtgggcctgagcgtgaccaagccctacgtgatcatgctgctggaggacggcaaggagccctggatgatggagaagaagctgagcaaggcctaccccttccccctgagccacagcgtg

SEQ ID NO: 926 - DNA, 240 bp, synthetic construct
  source 1..240: mol_type = other DNA
cccggccccctgagaagcctggagatggagagcctgcagttcagagatgtggccgtggagttcagcctggaggagtggcactgcctggacaccgcccagcagaacctgtacagagatgtgatgctggagaactacagacacctggtgttcctgggcatcatcgtgagcaagcccgacctgatcacctgcctggagcagggcatcaagcccctgaccatgaagagacacgagatgatcgcc

SEQ ID NO: 927 - DNA, 240 bp, synthetic construct
  source 1..240: mol_type = other DNA
ctgcagaccacctggccccaggagagcgtgaccttcgaggacgtggccgtgtacttcacccagaaccagtgggccagcctggaccccgcccagagagccctgtacggcgaggtgatgctggagaactacgccaacgtggccagcctggtggccttccccttccccaagcccgccctgatcagccacctggagagaggcgaggccccctggggacctgacccctgggacaccgagatcctg

SEQ ID NO: 928 - DNA, 240 bp, synthetic construct
  source 1..240: mol_type = other DNA
gcccccctgctggcccccagacccggcgagaccagacccggctgcagaaagcccggcacagtgagcttcgccgacgtggccgtgtacttcagccccgaggagtggggctgcctgagacccgcccagagagccctgtacagagatgtgatgcaggagacctacggccacctgggcgccctgggcttccccggccccaaacctgctctgatcagctggatggagcaggagagcgaggcctgg

SEQ ID NO: 929 - DNA, 240 bp, synthetic construct
  source 1..240: mol_type = other DNA
aacaaggtggagcagaagagccaggagagcgtgagcttcaaggacgtgaccgtgggcttcacccaggaggagtggcagcacctggaccccagccagagagccctgtacagagatgtgatgctggagaactacagcaacctggtgagcgtgggctactgcgtgcacaagcccgaggtgatcttcagactgcagcagggcgaggagccctggaagcaggaggaggagttccccagccagagc

SEQ ID NO: 930 - DNA, 240 bp, synthetic construct
  source 1..240: mol_type = other DNA
tgcttcagccaggaggagagaatggccgccggctacctgcccagatggagccaggagctggtgaccttcgaggacgtgagcatggacttcagccaggaggagtgggagctgctggagcccgcccagaagaacctgtacagagaggtgatgctggagaactacagaaacgtggtgagcctggaggccctgaagaaccagtgcaccgacgtgggcatcaaggagggccccctgagccccgcc

SEQ ID NO: 931 - DNA, 240 bp, synthetic construct
  source 1..240: mol_type = other DNA
gacagcgtggccttcgaggacgtggccgtgaacttcacccaggaggagtgggccctgctggaccccagccagaagaacctgtacagagaggtgatgcaggagaccctgagaaacctgaccagcatcggcaagaagtggaacaaccagtacatcgaggacgagcaccagaaccccagaagaaacctgagaagactgatcggcgagagactgagcgagagcaaggagagccaccagcacggc

SEQ ID NO: 932 - DNA, 240 bp, synthetic construct
  source 1..240: mol_type = other DNA
atgggccccgcccaggtgcccatgaacagcgaggtgatcgtggaccccatccagggccaggtgaacttcgaggacgtgttcgtgtacttcagccaggaggagtgggtgctgctggacgaggcccagagactgctgtacagagatgtgatgctggagaacttcgccctgatggccagcctgggccacaccagcttcatgagccacatcgtggccagcctggtgatgggcagcgagccctgg

SEQ ID NO: 933 - DNA, 240 bp, synthetic construct
  source 1..240: mol_type = other DNA
accaccttcaaggaggccatgaccttcaaggacgtggccgtggtgttcaccgaggaggagctgggcctgctggacctggcccagagaaagctgtacagagatgtgatgctggagaacttcagaaacctgctgagcgtgggccaccaggccttccacagagacaccttccacttcctgagagaggagaagatctggatgatgaagaccgccatccagagagagggcaacagcggcgacaag

SEQ ID NO: 934 - DNA, 240 bp, synthetic construct
  source 1..240: mol_type = other DNA
gacagcaccctgctgcagggcggccacaacctgctgagcagcgccagcttccaggaggccgtgaccttcaaggacgtgatcgtggacttcacccaggaggagtggaagcagctggaccccggccagagagacctgttcagagatgtgaccctggagaactacacccacctggtgagcatcggcctgcaggtgagcaagcccgacgtgatcagccagctggagcagggcaccgagccctgg

SEQ ID NO: 935 - DNA, 240 bp, synthetic construct
  source 1..240: mol_type = other DNA
gaggccagaagcatgctggtgcccccccaggccagcgtgtgcttcgaggacgtggccatggccttcacccaggaggagtgggagcagctggacctggcccagagaaccctgtacagagaggtgaccctggagacctgggagcacatcgtgagcctgggcctgttcctgagcaagagcgacgtgatcagccagctggagcaggaggaggacctgtgcagagccgagcaggaggcccccaga

SEQ ID NO: 936 - DNA, 240 bp, synthetic construct
  source 1..240: mol_type = other DNA
gacagcgtggtgttcgaggacgtggccgtggacttcaccctggaggagtgggccctgctggacagcgcccagagagacctgtacagagatgtgatgctggagaccttcagaaacctggccagcgtggacgacggcacccagttcaaggccaacggcagcgtgagcctgcaggacatgtacggccaggagaagagcaaggagcagaccatccccaacttcaccggcaacaacagctgcgcc

SEQ ID NO: 937 - DNA, 240 bp, synthetic construct
  source 1..240: mol_type = other DNA
gaggagagccacggcgccctgatcagcagctgcaacagcagaaccatgaccgacggcctggtgaccttcagagatgtggccatcgacttcagccaggaggagtgggagtgcctggaccccgcccagagagacctgtacgtggacgtgatgctggagaactacagcaacctggtgagcctggacctggagagcaagacctacgagaccaagaagatcttcagcgagaacgacatcttcgag

SEQ ID NO: 938 - DNA, 240 bp, synthetic construct
  source 1..240: mol_type = other DNA
gtgatcaccccccagatccccatggtgaccgaggagttcgtgaagcccagccagggccacgtgaccttcgaggacatcgccgtgtacttcagccaggaggagtggggcctgctggacgaggcccagagatgcctgtaccacgacgtgatgctggagaacttcagcctgatggccagcgtgggctgcctgcacggcatcgaggccgaggaggctcctagcgagcagaccctgagcgcccag

SEQ ID NO: 939 - DNA, 240 bp, synthetic construct
  source 1..240: mol_type = other DNA
gtgcagctgagaccccagaccagaatggccaccgccctgagagaccccgccagcggcagcgtgaccttcgaggacgtggccgtgtacttcagctgggaggagtgggacctgctggacgaggcccagaagcacctgtacttcgacgtgatgctggagaacttcgccctgaccagcagcctgggctgctggtgcggcgtggagcacgaggagacccccagcgagcagagaatcagcggcgag

SEQ ID NO: 940 - DNA, 240 bp, synthetic construct
  source 1..240: mol_type = other DNA
ggccccctgcagttcagagatgtggccatcgagttcagcctggaggagtggcactgcctggacgccgcccagagaaacctgtacagagatgtgatgctggagaactacagaaacctgatcttcctgggcatcgtggtgagcaagcccaacctgatcacctgcctggagcagggcaagaagcccctgaccatgaagagacacgagatgatcgccaagccccccgtgatgtacagccacttc

SEQ ID NO: 941 - DNA, 240 bp, synthetic construct
  source 1..240: mol_type = other DNA
atgcaggcccaggagagcctgaccctggaggacgtggccgtggacttcacctgggaggagtggcagttcctgagccccgcccagaaggacctgtacagagatgtgatgctggagaactacagcaacctggtggccgtgggctaccaggccagcaagcccgacgccctgagcaagctggagagaggcgaggagacctgcaccaccgaggacgagatctacagcagaatctgcagcgagatc

SEQ ID NO: 942 - DNA, 240 bp, synthetic construct
  source 1..240: mol_type = other DNA
ggccccctgcagttcagagatgtggccatcgagttcagcctggaggagtggcactgcctggacaccgcccagagaaacctgtacagagatgtgatgctggagaactacagaaacctggtgttcctgggcatcgtggtgagcaagcccgacctggtgacctgcctggagcagggcaagaagcccctgaccatggagagacacgagatgatcgccaagccccccgtgatgagcagccacttc

SEQ ID NO: 943 - DNA, 240 bp, synthetic construct
  source 1..240: mol_type = other DNA
aacatgttcaaggaggccgtgaccttcaaggacgtggccgtggccttcaccgaggaggagctgggcctgctgggccctgctcagagaaagctgtacagagatgtgatggtggagaacttcagaaacctgctgagcgtgggccacccccccttcaagcaggacgtgagccccatcgagagaaacgagcagctgtggatcatgaccaccgccaccagaagacagggcaacctgggcgagaag

SEQ ID NO: 944 - DNA, 240 bp, synthetic construct
  source 1..240: mol_type = other DNA
ggcgccctgaccttcagagatgtggccatcgagttcagcctggaggagtggcagtgcctggacaccgagcagcagaacctgtacagaaacgtgatgctggacaactacagaaacctggtgttcctgggcatcgccgtgagcaagcccgacctgatcacctgcctggagcaggagaaggagccctggaacctgaagacccacgacatggtggccaagccccccgtgatctgcagccacatc

SEQ ID NO: 945 - DNA, 240 bp, synthetic construct
  source 1..240: mol_type = other DNA
agcccccagaagagcagcgccctggcccccgaggaccacggcagcagctacgagggaagcgtgagcttcagagatgtggccatcgacttcagcagagaggagtggagacacctggaccccagccagagaaacctgtacagagatgtgatgctggagacctacagccacctgctgagcgtgggctaccaggtgcccgaggccgaggtggtgatgctggagcagggcaaggagccctgggcc

SEQ ID NO: 946 - DNA, 240 bp, synthetic construct
  source 1..240: mol_type = other DNA
gagctgctgaccttcagagatgtggccatcgagttcagccccgaggagtggaagtgcctggaccccgcccagcagaacctgtacagagatgtgatgctggagaactacagaaacctgatcagcctgggcgtggccatcagcaaccccgacctggtgatctacctggagcagagaaaggagccctacaaggtgaagatccacgagaccgtggccaagcaccccgccgtgtgcagccacttc

SEQ ID NO: 947 - DNA, 240 bp, synthetic construct
  source 1..240: mol_type = other DNA
gagcccctgaagttcagagatgtggccatcgagttcagcctggaggagtggcagtgcctggacaccatccagcagaacctgtacagaaacgtgatgctggagaactacagaaacctggtgttcctgggcatcgtggtgagcaagcccgacctgatcacctgcctggagcaggagaaggagccctggaccagaaagagacacagaatggtggccgagccccccgtgatctgcagccacttc

SEQ ID NO: 948 - DNA, 240 bp, synthetic construct
  source 1..240: mol_type = other DNA
cccagcgccagaggcaagagcaagagcaaggcccccatcaccttcggcgacctggccatctacttcagccaggaggagtgggagtggctgagccccatccagaaggacctgtacgaggacgtgatgctggagaactacagaaacctggtgagcctgggcctgagcttcagaagacccaacgtgatcaccctgctggagaagggcaaggccccctggatggtggagcccgtgagaagaaga

SEQ ID NO: 949 - DNA, 240 bp, synthetic construct
  source 1..240: mol_type = other DNA
accaaggcccaggagagcctgaccctggaggacgtggccgtggacttcacctgggaggagtggcagttcctgagccccgcccagaaggacctgtacagagatgtgatgctggagaactacagcaacctggtgagcgtgggctaccaggccggcaagcccgacgccctgaccaagctggagcagggcgagcctctgtggaccctggaggacgagatccacagccccgcccaccccgagatc

SEQ ID NO: 950 - DNA, 240 bp, synthetic construct
  source 1..240: mol_type = other DNA
agccaggaggaggtggaggtggccggcatcaagctgtgcaaggccatgagcctgggcagcgtgaccttcaccgacgtggccatcgacttcagccaggacgagtgggagtggctgaacctggcccagagaagcctgtacaagaaggtgatgctggagaactacagaaacctggtgagcgtgggcctgtgcatcagcaagcccgacgtgatcagcctgctggagcaggagaaggacccctgg

SEQ ID NO: 951 - DNA, 240 bp, synthetic construct
  source 1..240: mol_type = other DNA
accaagagcctggagagcgtgagcttcaaggacgtgaccgtggacttcagcagagatgagtggcagcagctggacctggcccagaagagcctgtacagagaggtgatgctggagaactacttcaacctgatcagcgtgggctgccaggtgcccaagcccgaggtgatcttcagcctggagcaggaggagccctgcatgctggacggcgagatccccagccagagcagacccgacggcgac

SEQ ID NO: 952 - DNA, 240 bp, synthetic construct
  source 1..240: mol_type = other DNA
agcggctgccccggcgccgagagaaacctgctggtgtacagctacttcgagaaggagaccctgaccttcagagatgtggccatcgagttcagcctggaggagtgggagtgcctgaaccccgcccagcagaacctgtacatgaacgtgatgctggagaactacaagaacctggtgttcctgggcgtggccgtgagcaagcaggaccccgtgacctgcctggagcaggagaaggagccctgg

SEQ ID NO: 953 - DNA, 240 bp, synthetic construct
  source 1..240: mol_type = other DNA
cccctgcagggcagcgtgagcttcaaggacgtgaccgtggacttcacccaggaggagtggcagcagctggaccccgcccagaaggccctgtacagagatgtgatgctggagaactactgccacttcgtgagcgtgggcttccacatggccaagcccgacatgatcagaaagctggagcagggcgaggagctgtggacccagagaatcttccccagctacagctacctggaggaggacggc

SEQ ID NO: 954 - DNA, 240 bp, synthetic construct
  source 1..240: mol_type = other DNA
cccggcccccccagaagcctggagatgggcctgctgaccttcagagatgtggccatcgagttcagcctggaggagtggcagcacctggacatcgcccagcagaacctgtacagaaacgtgatgctggagaactacagaaacctggccttcctgggcatcgccgtgagcaagcccgacctgatcacctgcctggagcagggcaaggagccctggaacatgaagagacacgagatggtggac

SEQ ID NO: 955 - DNA, 240 bp, synthetic construct
  source 1..240: mol_type = other DNA
agcggccaccccggcagctgggagatgaacagcgtggccttcgaggacgtggccgtgaacttcacccaggaggagtgggccctgctggaccccagccagaagaacctgtacagagatgtgatgcaggagaccttcagaaacctggccagcatcggcaacaagggcgaggaccagagcatcgaggaccagtacaagaacagcagcagaaacctgagacacatcatcagccacagcggcaac

SEQ ID NO: 956 - DNA, 240 bp, synthetic construct
  source 1..240: mol_type = other DNA
agctacggcagcatcaccttcggcgacgtggccatcgacttcagccaccaggagtgggagtacctgagcctggtgcagaagaccctgtaccaggaggtgatgatggagaactacgacaacctggtgagcctggccggccacagcgtgagcaagcccgacctgatcaccctgctggagcagggcaaggagccctggatgatcgtgagagaggagaccagaggcgagtgcaccgacctggac

SEQ ID NO: 957 - DNA, 240 bp, synthetic construct
  source 1..240: mol_type = other DNA
gacctgttcgtgtgcagcggcctggagccccacacccccagcgtgggcagccaggaaagcgtgaccttccaggacgtggccgtggacttcaccgagaaggagtggcccctgctggacagcagccagagaaagctgtacaaggacgtgatgctggagaactacagcaacctgaccagcctgggctaccaggtgggcaagcccagcctgatcagccacctggagcaggaggaggagcccaga

SEQ ID NO: 958 - DNA, 240 bp, synthetic construct
  source 1..240: mol_type = other DNA
ttccagaccgcctggagacaggagcccgtgaccttcgaggacgtggccgtgtacttcacccagaacgagtgggccagcctggacagcgtgcagagagccctgtacagagaggtgatgctggagaactacgccaacgtggccagcctggccttccccttcaccacccccgtgctggtgagccagctggagcagggcgagctgccctggggactggacccctgggagcctatgggcagagag

SEQ ID NO: 959 - DNA, 240 bp, synthetic construct
  source 1..240: mol_type = other DNA
gagctgctgaccttcagagatgtggccatcgagttcagccccgaggagtggaagtgcctggaccccgaccagcagaacctgtacagagatgtgatgctggagaactacagaaacctggtgagcctgggcgtggccatcagcaaccccgacctggtgacctgcctggagcagagaaaggagccctacaacgtgaagatccacaagatcgtggccagaccccccgccatgtgcagccacttc

SEQ ID NO: 960 - DNA, 240 bp, synthetic construct
  source 1..240: mol_type = other DNA
gccggcgaggccgaggcccagctggaccccagcctgcagggcctggttatgttcgaggacgtgaccgtgtacttcagcagagaggagtggggcctgctgaacgtgacccagaagggcctgtacagagatgtgatgctggagaacttcgccctggtgagcagcctgggcctggcccccagcagaagccccgtgttcacccagctggaggacgacgagcagagctgggtgcccagctgggtg

SEQ ID NO: 961 - DNA, 240 bp, synthetic construct
  source 1..240: mol_type = other DNA
gcccacgccctggtgaccttcagagatgtggccatcgacttcagccagaaggagtgggagtgcctggacaccacccagagaaagctgtacagagatgtgatgctggagaactacaacaacctggtgagcctgggctacagcggcagcaagcccgacgtgatcaccctgctggagcagggcaaggagccctgcgtggtggccagagatgtgaccggcagacagtgccccggcctgctgagc

SEQ ID NO: 962 - DNA, 240 bp, synthetic construct
  source 1..240: mol_type = other DNA
aagagaatcaagcactggaagatggccagcaagctgatcctgcccgagagcctgagcctgctgaccttcgaggacgtggccgtgtacttcagcgaggaggagtggcagctgctgaaccccctggagaagaccctgtacaacgacgtgatgcaggacatctacgagaccgtgatcagcctgggcctgaagctgaagaacgacaccggcaacgaccaccccatcagcgtgagcaccagcgag

SEQ ID NO: 963 - DNA, 240 bp, synthetic construct
  source 1..240: mol_type = other DNA
gacagcgtggtgttcgaggacgtggccgtggacttcaccctggaggagtgggccctgctggacagcgcccagagagacctgtacagagatgtgatgctggagaccttccagaacctggccagcgtggacgacgagacccagttcaaggccagcggcagcgtgagccagcaggacatctacggcgagaagatccccaaggagagcaagatcgccaccttcaccagaaacgtgagctgggcc

SEQ ID NO: 964 - DNA, 240 bp, synthetic construct
  source 1..240: mol_type = other DNA
aacatgagccaggccagcgtgagcttccaggacgtgaccgtggagttcaccagagaggagtggcagcacctgggccccgtggagagaaccctgtacagagatgtgatgctggagaactacagccacctgatcagcgtgggctactgcatcaccaagcccaaggtgatcagcaagctggagaagggcgaggagccctggagcctggaggacgagttcctgaaccagagataccccggctac

SEQ ID NO: 965 - DNA, 240 bp, synthetic construct
  source 1..240: mol_type = other DNA
atcagcttccaggagagcgtgaccttccaggacgtggccgtggacttcaccgccgaggagtggcagctgctggactgcgccgagagaaccctgtactgggacgtgatgctggagaactacagaaacctgatcagcgtgggctgccccatcaccaagaccaaggtgatcctgaaggtggagcagggccaggagccctggatggtggagggcgccaacccccacgagagcagccccgagagc

SEQ ID NO: 966 - DNA, 240 bp, synthetic construct
  source 1..240: mol_type = other DNA
aacaccctgcagggccccgtgagcttcaaggacgtggccgtggacttcacccaggaggagtggcagcagctggaccccgacgagaagatcacctacagagatgtgatgctggagaactacagccacctggtgagcgtgggctacgacaccaccaagcccaacgtgatcatcaagctggagcagggcgaggagccctggatcatgggcggcgagttcccctgccagcacagccccgaggcc

SEQ ID NO: 967 - DNA, 240 bp, synthetic construct
  source 1..240: mol_type = other DNA
caccccgaggaggaggagagaatgcacgacgagctgctgcaggccgtgagcaagggccccgtgatgttcagagatgtgagcatcgacttcagccaggaggagtgggagtgcctggacgccgaccagatgaacctgtacaaggaggtgatgctggagaacttcagcaacctggtgagcgtgggcctgagcaacagcaagcccgccgtgatcagcctgctggagcagggcaaggagccctgg

SEQ ID NO: 968 - DNA, 240 bp, synthetic construct
  source 1..240: mol_type = other DNA
agcctgggcagcgagctgttcagagatgtggccatcgtgttcagccaggaggagtggcagtggctggcccccgcccagagagacctgtacagagatgtgatgctggagacctacagcaacctggtgagcctgggcctggccgtgagcaagcccgacgtgatcagcttcctggagcagggcaaggagccctggatggtggagagagtggtgagcggcggcctgtgccccgtgctggagagc

SEQ ID NO: 969 - DNA, 240 bp, synthetic construct
  source 1..240: mol_type = other DNA
accaagttccaggagatggtgaccttcaaggacgtggccgtggtgttcaccgaggaggagctgggcctgctggacagcgtgcagagaaagctgtacagagatgtgatgctggagaacttcagaaacctgctgctggtggcccaccagcccttcaagcccgacctgatcagccagctggagagagaggagaagctgctgatggtggagaccgagacccccagagatggctgcagcggcaga

SEQ ID NO: 970 - DNA, 240 bp, synthetic construct
  source 1..240: mol_type = other DNA
gccaagagacccggcccccccggcagcagagagatgggcctgctgaccttcagagacatcgccatcgagttcagcctggccgagtggcagtgcctggaccacgcccagcagaacctgtacagagatgtgatgctggagaactacagaaacctggtgagcctgggcatcgccgtgagcaagcccgacctgatcacctgcctggagcagaacaaggagccccagaacatcaagagaaacgag

SEQ ID NO: 971 - DNA, 240 bp, synthetic construct
  source 1..240: mol_type = other DNA
acctgcatggtgcacagacagaccatgagctgcagcggcgccggcggcatcaccgccttcgtggccttcagagatgtggccgtgtacttcacccaggaggagtggagactgctgagccccgcccagagaaccctgcacagagaggtgatgctggagacctacaaccacctggtgagcctggagatccccagcagcaagcccaagctgatcgcccagctggagagaggcgaggccccctgg

SEQ ID NO: 972 - DNA, 240 bp, synthetic construct
  source 1..240: mol_type = other DNA
atccaggcccaggagagcatcaccctggaggacgtggccgtggacttcacctgggaggagtggcagctgctgggcgccgcccagaaggacctgtacagagatgtgatgctggagaactacagcaacctggtggccgtgggctaccaggccagcaagcccgacgccctgttcaagctggagcagggcgagcagctgtggaccatcgaggacggcatccacagcggcgcctgcagcgacatc

SEQ ID NO: 973 - DNA, 240 bp, synthetic construct
  source 1..240: mol_type = other DNA
gcccagaagagaagaaagagaaaggccaaggagagcggcatggccctgccccagggccacctgaccttcagagatgtggccatcgagttcagccaggccgagtggaagtgcctggaccccgcccagagagccctgtacaaggacgtgatgctggagaactacagaaacctggtgagcctgggcatcagcctgcccgacctgaacatcaacagcatgctggagcagagaagagagccctgg

SEQ ID NO: 974 - DNA, 240 bp, synthetic construct
  source 1..240: mol_type = other DNA
gacagcaccagcgtgcccgtgaccgccgaggccaagctgatgggcttcacccagggctgcgtgaccttcgaggacgtggccatctacttcagccaggaggagtggggcctgctggacgaggcccagagactgctgtacagagatgtgatgctggagaacttcgccctgatcaccgccctggtgtgctggcacggcatggaggacgaggagacccccgagcagagcgtgagcgtggagggc

SEQ ID NO: 975 - DNA, 240 bp, synthetic construct
  source 1..240: mol_type = other DNA
ggccccctgaccttcagagatgtgaagatcgagttcagcctggaggagtggcagtgcctggacaccgcccagagaaacctgtacagagatgtgatgctggagaactacagaaacctggtgttcctgggcatcgccgtgagcaagcccgacctgatcacctggctggagcagggcaaggagccctggaacctgaagagacacgagatggtggacaagacccccgtgatgtgcagccacttc

SEQ ID NO: 976 - DNA, 240 bp, synthetic construct
  source 1..240: mol_type = other DNA
agcggctgccccggcgccgagagaagcctgctggtgcagagctacttcgagaagggccccctgaccttcagagatgtggccatcgagttcagcctggaggagtggcagtgcctggacagcgcccagcagggcctgtacagaaaggtgatgctggagaactacagaaacctggtgttcctggccggcatcgccctgaccaagcccgacctgatcacctgcctggagcagggcaaggagccc

SEQ ID NO: 977 - DNA, 240 bp, synthetic construct
  source 1..240: mol_type = other DNA
ggcgtgctgaccttcagagatgtggccgtggagttcagccccgaggagtgggagtgcctggacagcgcccagcagagactgtacagagatgtgatgctggagaactacggcaacctggtgagcctgggcctggccatcttcaagcccgacctgatgacctgcctggagcagagaaaggagccctggaaggtgaagagacaggaggccgtggccaagcaccccgccggcagcttccacttc

SEQ ID NO: 978 - DNA, 240 bp, synthetic construct
  source 1..240: mol_type = other DNA
aaggagaacctggaggacatcagcggctggggcctgcccgaggccagaagcaaggagagcgtgagcttcaaggacgtggccgtggacttcacccaggaggagtggggccagctggacagcccccagagagccctgtacagagatgtgatgctggagaactaccagaacctgctggccctgggcccccccctgcacaagcccgacgtgatcagccacctggagagaggcgaggagccctgg

SEQ ID NO: 979 - DNA, 240 bp, synthetic construct
  source 1..240: mol_type = other DNA
gaggagagcgagaagcccagaggcttcgccagaggcctggagcccgagagaatcatcggcgccaccgacagcagcggcgagctgatgttcctgatgaagtggaagaacagcgacgaggccgacctggtgcccgccaaggaggccaacgtgaagtgcccccaggtggtgatcagcttctacgaggagagactgacctggcacagctaccccagcgaggacgacgacaagaaggacgacaag

SEQ ID NO: 980 - DNA, 240 bp, synthetic construct
  source 1..240: mol_type = other DNA
gccagcgtggccctggaggacgtggccgtgaacttcaccagagaggagtgggccctgctgggcccctgccagaagaacctgtacaaggacgtgatgcaggagaccatcagaaacctggactgcgtggtgatgaagtggaaggaccagaacatcgaggaccagtacagataccccagaaagaacctgagatgcagaatgctggagagattcgtggagagcaaggacggcacccagtgcggc

SEQ ID NO: 981 - DNA, 240 bp, synthetic construct
  source 1..240: mol_type = other DNA
accctgctgaccttcagagatgtggccatcgagttcagcctggaggagtggaagtgcctggacctggcccagcagaacctgtacagagatgtgatgctggagaactacagaaacctgttcagcgtgggcctgaccgtgtgcaagcccggcctgatcacctgcctggagcagagaaaggagccctggaacgtgaagagacaggaggccgccgacggccaccccgagatgggcttccaccac

SEQ ID NO: 982 - DNA, 240 bp, synthetic construct
  source 1..240: mol_type = other DNA
gccgccgccctgagagcccccacccagcaggtgttcgtggccttcgaggacgtggccatctacttcagccaggaggagtgggagctgctggacgagatgcagagactgctgtacagagatgtgatgctggagaacttcgccgtgatggccagcctgggctgctggtgcggcgccgtggacgagggaacacctagcgccgagagcgtgagcgtggaggagctgagccagggcagaaccccc

SEQ ID NO: 983 - DNA, 240 bp, synthetic construct
  source 1..240: mol_type = other DNA
aacaccttccaggccagcgtgagcttccaggacgtgaccgtggagttcagccaggaggagtggcagcacatgggccccgtggagagaaccctgtacagagatgtgatgctggagaactacagccacctggtgagcgtgggctactgcttcaccaagcccgagctgatcttcaccctggagcagggcgaggacccctggctgctggagaaggagaagggcttcctgagcagaaacagcccc

SEQ ID NO: 984 - DNA, 240 bp, synthetic construct
  source 1..240: mol_type = other DNA
gacagcgtggccttcgaggacgtgagcgtgagcttcagccaggaggagtgggccctgctggcccccagccagaagaagctgtacagagatgtgatgcaggagaccttcaagaacctggccagcatcggcgagaagtgggaggaccccaacgtggaggaccagcacaagaaccagggcagaaacctgagaagccacaccggcgagagactgtgcgagggcaaggagggcagccagtgcgcc

SEQ ID NO: 985 - DNA, 240 bp, synthetic construct
  source 1..240: mol_type = other DNA
gcccagggcctggtgaccttcgccgacgtggccatcgacttcagccaggaggagtgggcctgcctgaacagcgcccagagagacctgtactgggacgtgatgctggagaactacagcaacctggtgagcctggacctggagagcgcctacgagaacaagagcctgcccaccgagaagaacatccacgagatcagagccagcaagagaaacagcgacagaagaagcaagagcctgggcaga

SEQ ID NO: 986 - DNA, 240 bp, synthetic construct
  source 1..240: mol_type = other DNA
gccgtggacctgctgagcgcccaggagcccgtgaccttcagagatgtggccgtgttcttcagccaggacgagtggctgcacctggacagcgcccagagagccctgtacagagaggtgatgctggagaactacagcagcctggtgagcctgggcatccccttcagcatgcccaagctgatccaccagctgcagcagggcgaggacccctgcatggtggagagagaggtgcccagcgacacc

SEQ ID NO: 987 - DNA, 240 bp, synthetic construct
  source 1..240: mol_type = other DNA
agcccccagagattccccgccctgatccccggcgagcccggcagaagcttcgagggcagcgtgagcttcgaggacgtggccgtggacttcaccagacaggagtggcacagactggaccccgcccagagaaccatgcacaaggacgtgatgctggagacctacagcaacctggccagcgtgggcctgtgcgtggccaagcccgagatgatcttcaagctggagagaggcgaggagctgtgg

SEQ ID NO: 988 - DNA, 240 bp, synthetic construct
  source 1..240: mol_type = other DNA
agagtgctgaccttcagagatgtggccgtggagttcagccccgaggagtgggagtgcctggacagcgcccagcagagactgtacagagatgtgatgctggagaactacggcaacctgttcagcctgggcctggccatcttcaagcccgacctgatcacctacctggagcagagaaaggagccctggaacgccagaagacagaagaccgtggccaagcaccccgccggcagcctgcacttc

SEQ ID NO: 989 - DNA, 240 bp, synthetic construct
  source 1..240: mol_type = other DNA
gccgagaccaaggacgccgcccagatgctggtgaccttcaaggacgtggccgtgaccttcaccagagaggagtggagacagctggacctggcccagagaaccctgtacagagaggtgatgctggagacctgcggcctgctggtgagcctgggccacagagtgcccaagcccgagctggtgcacctgctggagcacggccaggagctgtggatcgtgaagagaggcctgagccacgccacc

SEQ ID NO: 990 - DNA, 240 bp, synthetic construct
  source 1..240: mol_type = other DNA
atcgagtaccagatccccgtgagcttcaaggacgtggtggtgggcttcacccaggaggagtggcacagactgagccccgcccagagagccctgtacagagatgtgatgctggagacctacagcaacctggtgagcgtgggctacgagggcaccaagcccgacgtgatcctgagactggagcaggaggaggccccctggatcggcgaggccgcttgccctggatgccactgctgggaggac

SEQ ID NO: 991 - DNA, 240 bp, synthetic construct
  source 1..240: mol_type = other DNA
accaagttccaggaggccgtgaccttcaaggacgtggccgtggccttcaccgaggaggagctgggcctgctggacagcgcccagagaaagctgtacagagatgtgatgctggagaacttcagaaacctggtgagcgtgggccaccagagcttcaagcccgacatgatcagccagctggagagagaggagaagctgtggatgaaggagctgcagacccagagaggcaagcacagcggcgac

SEQ ID NO: 992 - DNA, 240 bp, synthetic construct
  source 1..240: mol_type = other DNA
gacgagggcgtggccggcgtgatgagcgtgggcccccccgctgctagacttcaggagcccgtgaccttcagagatgtggccgtggacttcacccaggaggagtggggccagctggaccccacccagagaatcctgtacagagatgtgatgctggagaccttcggccacctgctgagcatcggccccgagctgcccaagcccgaggtgatcagccagctggagcagggcaccgagctgtgg

SEQ ID NO: 993 - DNA, 240 bp, synthetic construct
  source 1..240: mol_type = other DNA
ggccccctgaccttcatggacgtggccatcgagttcagcgtggaggagtggcagtgcctggacaccgcccagcagaacctgtacagaaacgtgatgctggagaactacagaaacctggtgttcctgggcatcgccgtgagcaagcccgacctgatcacctgcctggagcagggcaaggagccctggaacatggagagacacgagatggtggccaagccccccggcatgtgctgctacttc

SEQ ID NO: 994 - DNA, 240 bp, synthetic construct
  source 1..240: mol_type = other DNA
caccaggtgggcctgatcagaagctacaacagcaagaccatgacctgcttccaggagctggtgaccttcagagatgtggccatcgacttcagcagacaggagtgggagtacctggaccccaaccagagagacctgtacagagatgtgatgctggagaactacagaaacctggtgagcctgggcggccacagcatcagcaagcccgtggtggtggacctgctggagagaggcaaggagccc

SEQ ID NO: 995 - DNA, 240 bp, synthetic construct
  source 1..240: mol_type = other DNA
aacgccaccatcgtgatgagcgtgagaagagagcagggcagcagcagcggcgagggcagcctgagcttcgaggacgtggccgtgggcttcaccagagaggagtggcagttcctggaccagagccagaaggtgctgtacaaggaggtgatgctggagaactacatcaacctggtgagcatcggctacagaggcaccaagcccgacagcctgttcaagctggagcagggcgagccccccggc

SEQ ID NO: 996 - DNA, 240 bp, synthetic construct
  source 1..240: mol_type = other DNA
ttcccccccgccagaggcaaggagctgctgagcttcgaggacgtggccatgtacttcaccagagaggagtggggccacctgaactggggccagaaggacctgtacagagatgtgatgctggagaactacagaaacatggtgctgctgggcttccagttccccaagcccgagatgatctgccagctggagaactgggacgagcagtggatcctggacctgcccagaaccggcaacagaaag

SEQ ID NO: 997 - DNA, 240 bp, synthetic construct
  source 1..240: mol_type = other DNA
gagctgctgaccttcaaggacgtggccatcgagttcagccccgaggagtggaagtgcctggacaccagccagcagaacctgtacagagatgtgatgctggagaactacagaaacctggtgagcctgggcgtgagcatcagcaaccccgacctggtgaccagcctggagcagagaaaggagccctacaacctgaagatccacgagaccgccgccagaccccccgccgtgtgcagccacttc

SEQ ID NO: 998 - DNA, 240 bp, synthetic construct
  source 1..240: mol_type = other DNA
atgaagagccagggcctggtgagcttcaaggacgtggccgtggacttcacccaggaggagtggcagcagctggaccccagccagagaaccctgtacagagatgtgatgctggagaactacagccacctggtgagcatgggctaccccgtgagcaagcccgacgtgatcagcaagctggagcagggcgaggagccctggatcatcaagggcgacatcagcaactggatctaccccgacgag

SEQ ID NO: 999 - DNA, 240 bp, synthetic construct
  source 1..240: mol_type = other DNA
gccgagggcagcgtgatgttcagcgacgtgagcatcgacttcagccaggaggagtgggactgcctggaccccgtgcagagagacctgtacagagatgtgatgctggagaactacggcaacctggtgagcatgggcctgtacacccccaagccccaggtgatcagcctgctggagcagggcaaggagccctggatggtgggcagagagctgaccagaggcctgtgcagcgacctggagagc

SEQ ID NO: 1000 - DNA, 240 bp, synthetic construct
  source 1..240: mol_type = other DNA
ggccccctgaccttcaccgacgtggccatcgagttcagcctggaggagtggcagtgcctggacaccgcccagcagaacctgtacagaaacgtgatgctggagaactacagaaacctggtgttcctgggcatcgccgtgagcaagcccgacctgatcacctgcctggagaaggagaaggagccctgcaagatgaagagacacgagatggtggacgagccccccgtggtgtgcagccacttc

SEQ ID NO: 1001 - DNA, 240 bp, synthetic construct
  source 1..240: mol_type = other DNA
gccctgagccagggcctgttcaccttcaaggacgtggccatcgagttcagccaggaggagtgggagtgcctggaccccgcccagagagccctgtacagagatgtgatgctggagaactacagaaacctgctgagcctggacgaggacaacatcccccccgaggacgacatcagcgtgggcttcaccagcaagggcctgagccccaaggagaacaacaaggaggagctgtaccacctggtg

SEQ ID NO: 1002 - DNA, 240 bp, synthetic construct
  source 1..240: mol_type = other DNA
aacatggagggcctggtgatgttccaggacctgagcatcgacttcagccaggaggagtgggagtgcctggacgccgcccagaaggacctgtacagagatgtgatgatggagaactacagcagcctggtgagcctgggcctgagcatccccaagcccgacgtgatcagcctgctggagcagggcaaggagccctggatggtgagcagagatgtgctgggcggctggtgcagagacagcgag

SEQ ID NO: 1003 - DNA, 240 bp, synthetic construct
  source 1..240: mol_type = other DNA
gccgtgagccacctgcccaccatggtgcaggagagcgtgaccttcaaggacgtggccatcctgttcacccaggaggagtggggccagctgagccccgcccagagagccctgtacagagatgtgatgctggagaactacagcaacctggtgagcctgggcctgctgggccccaagcccgacaccttcagccagctggagaagagagaggtgtggatgcccgaggacacccccggcggcttc

SEQ ID NO: 1004 - DNA, 240 bp, synthetic construct
  source 1..240: mol_type = other DNA
ggccccctgaccatcagagatgtgaccgtggagttcagcctggaggagtggcactgcctggacaccgcccagcagaacctgtacagagatgtgatgctggagaactacagaaacctggtgttcctgggcatcgccgtgagcaagcccgacctgatcacctgcctggagcagggcaaggagccctgcaacatgaagagacacgagatggtggccaagccccccgtgatgtgcagccacatc

SEQ ID NO: 1005 - DNA, 240 bp, synthetic construct
  source 1..240: mol_type = other DNA
gccgccgccgtgctgaccgacagagcccaggtgagcgtgaccttcgacgacgtggccgtgaccttcaccaaggaggagtggggccagctggacctggcccagagaaccctgtaccaggaggtgatgctggagaactgcggcctgctggtgagcctgggctgccccgtgcccaaggccgagctgatctgccacctggagcacggccaggagccctggaccagaaaggaggacctgagccag

SEQ ID NO: 1006 - DNA, 240 bp, synthetic construct
  source 1..240: mol_type = other DNA
gccctgagaagcgtgatgttcagcgacgtgagcatcgacttcagccccgaggagtgggagtacctggacctggagcagaaggacctgtacagagatgtgatgctggagaactacagcaacctggtgagcctgggctgcttcatcagcaagcccgacgtgatcagcagcctggagcagggcaaggagccctggaaggtggtgagaaagggcagaagacagtaccccgacctggagaccaag

SEQ ID NO: 1007 - DNA, 240 bp, synthetic construct
  source 1..240: mol_type = other DNA
gcccacggcagcgtgaccttcagagatgtggccatcgacttcagccaggaggagtgggagttcctggaccccgcccagagagacctgtacagagatgtgatgtgggagaactacagcaacttcatcagcctgggccccagcatcagcaagcccgacgtgatcaccctgctggacgaggagagaaaggagcccggcatggtggtgagagagggcaccagaagatactgccccgacctggag

SEQ ID NO: 1008 - DNA, 240 bp, synthetic construct
  source 1..240: mol_type = other DNA
gcccccagagcccagatccagggccccctgaccttcggcgacgtggccgtggccttcaccagaatcgagtggagacacctggacgccgcccagagagccctgtacagagatgtgatgctggagaactacggcaacctggtgagcgtgggcctgctgagcagcaagcccaagctgatcacccagctggagcagggcgccgagccctggaccgaggtgagagaggcccctagcggcacacac

SEQ ID NO: 1009 - DNA, 240 bp, synthetic construct
  source 1..240: mol_type = other DNA
ggccccctgcagttcagagatgtggccatcgagttcagcctggaggagtggcactgcctggacaccgcccagagaaacctgtacagaaacgtgatgctggagaactacagaaacctggtgttcctgggcatcgtggtgagcaagcccgacctgatcacctgcctggagcagggcaagaagcccctgaccatgaagaagcacgagatggtggccaaccccagcgtgacctgcagccacttc

SEQ ID NO: 1010 - DNA, 240 bp, synthetic construct
  source 1..240: mol_type = other DNA
accctgcccagaggccagcccgaggtgctggagtggggcctgcccaaggaccaggacagcgtggccttcgaggacgtggccgtgaacttcacccacgaggagtgggccctgctgggccccagccagaagaacctgtacagagatgtgatgagagagaccatcagaaacctgaactgcatcggcatgaagtgggagaaccagaacatcgacgaccagcaccagaacctgagaagaaacccc

SEQ ID NO: 1011 - DNA, 240 bp, synthetic construct
  source 1..240: mol_type = other DNA
cccagccccgacagcatgaccttcgaggacatcatcgtggacttcacccaggaggagtgggccctgctggacaccagccagagaaagctgttccaggacgtgatgctggagaacatcagccacctggtgagcatcggcaagcagctgtgcaagagcgtggtgctgagccagctggagcaggtggagaagctgagcacccagagaatcagcctgctgcagggcagagaggtgggcatcaag

SEQ ID NO: 1012 - DNA, 240 bp, synthetic construct
  source 1..240: mol_type = other DNA
gaggagagcagagagatcagagccggccagatcgtgctgaaggccatggcccagggcctggtgaccttcagagatgtggccatcgagttcagcctggaggagtggaagtgcctggagcccgcccagagagacctgtacagagaggtgaccctggagaacttcggccacctggccagcctgggcctgagcatcagcaagcccgacgtggtgagcctgctggagcagggcaaggagccctgg

SEQ ID NO: 1013 - DNA, 240 bp, synthetic construct
  source 1..240: mol_type = other DNA
gccgccagcgcccaggtgagcgtgaccttcgaggacgtggccgtgaccttcacccaggaggagtggggccagctggacgccgcccagagaaccctgtaccaggaggtgatgctggagacctgcggcctgctgatgagcctgggctgccccctgttcaagcccgagctgatctaccagctggaccacagacaggagctgtggatggccaccaaggacctgagccagagcagctaccccggc

SEQ ID NO: 1014 - DNA, 240 bp, synthetic construct
  source 1..240: mol_type = other DNA
agagagagcctggaggacgaggtgacccccggcctgcccaccgccgagagccaagagctgctgaccttcaaggacatcagcatcgacttcacccaggaggagtggggccagctggcccccgcccaccagaacctgtacagagaggtgatgctggagaactacagcaacctggtgagcgtgggctaccagctgagcaagcccagcgtgatcagccagctggagaagggcgaggagccctgg

SEQ ID NO: 1015 - DNA, 240 bp, synthetic construct
  source 1..240: mol_type = other DNA
gagggcgagtacatcaagctgaaggtgatcggccaggacagcagcgagatccacttcaaggtgaagatgaccacccacctgaagaagctgaaggagagctactgccagagacagggcgtgcccatgaacagcctgagattcctgttcgagggccagagaatcgccgacaaccacacccccaaggagctgggcatggaggaggaggacgtgatcgaggtgtaccaggagcagaccggcggc

SEQ ID NO: 1016 - DNA, 240 bp, synthetic construct
  source 1..240: mol_type = other DNA
aacaagagcctgggccccgtgagcttcaaggacgtggccgtggacttcacccaggaggagtggcagcagctggaccccgagcagaagatcacctacagagatgtgatgctggagaactacagcaacctggtgagcgtgggctaccacatcatcaagcccgacgtgatcagcaagctggagcagggcgaggagccctggatcgtggagggcgagttcctgctgcagagctaccccgacgag

SEQ ID NO: 1017 - DNA, 240 bp, synthetic construct
  source 1..240: mol_type = other DNA
agccccggcctgctgaccaccagaaaggaggccctgatggccttcagagatgtggccgtggccttcacccagaaggagtggaagctgctgagcagcgcccagagaaccctgtacagagaggtgatgctggagaactacagccacctggtgagcctgggcatcgccttcagcaagcccaagctgatcgagcagctggagcagggcgacgagccctggagagaggagaacgagcacctgctg

SEQ ID NO: 1018 - DNA, 240 bp, synthetic construct
  source 1..240: mol_type = other DNA
atgttccaggacagcgtggccttcgaggacgtggccgtgaccttcacccaggaggagtgggccctgctggaccccagccagaagaacctgtgcagagatgtgatgcaggagaccttcagaaacctggccagcatcggcaagaagtggaagccccagaacatctacgtggagtacgagaacctgagaagaaacctgagaatcgtgggcgagagactgttcgagagcaaggagggccaccag

SEQ ID NO: 1019 - DNA, 240 bp, synthetic construct
  source 1..240: mol_type = other DNA
gagaacgaccacatcaacctgaaggtggccggccaggacggcagcgtggtgcagttcaagatcaagagacacacccccctgagcaagctgatgaaggcctactgcgagagacagggcctgagcatgagacagatcagattcagattcgacggccagcccatcaacgagaccgacacccccgcccagctggagatggaggacgaggacaccatcgacgtgttccagcagcagaccggcggc

SEQ ID NO: 1020 - DNA, 240 bp, synthetic construct
  source 1..240: mol_type = other DNA
cccggccccctgggcagcctggagatgggcgtgctgaccttcagagatgtggccctggagttcagcctggaggagtggcagtgcctggacaccgcccagcagaacctgtacagaaacgtgatgctggagaactacagaaacctggtgttcgtgggcatcgccgccagcaagcccgacctgatcacctgcctggagcagggcaaggagccctggaacgtgaagagacacgagatggtgacc

SEQ ID NO: 1021 - DNA, 240 bp, synthetic construct
  source 1..240: mol_type = other DNA
ctgagccagaagccccaggtgctgggccccgagaagcaggacggcagctgcgaggctagcgtgagcttcgaggacgtgaccgtggacttcagcagagaggagtggcagcagctggaccccgcccagagatgcctgtacagagatgtgatgctggagctgtacagccacctgttcgccgtgggctaccacatccccaaccccgaggtgatcttcagaatgctgaaggagaaggagcccaga

SEQ ID NO: 1022 - DNA, 240 bp, synthetic construct
  source 1..240: mol_type = other DNA
gccctgacccagggccaggtgaccttcagagatgtggccatcgagttcagccaggaggagtggacctgcctggaccccgcccagagaaccctgtacagagatgtgatgctggagaactacagaaacctggccagcctgggcatcagctgcttcgacctgagcatcatcagcatgctggagcagggcaaggagcccttcaccctggagagccaggtgcagatcgccggcaaccccgacggc

SEQ ID NO: 1023 - DNA, 240 bp, synthetic construct
  source 1..240: mol_type = other DNA
aacaagttccagggccccgtgaccctgaaggacgtgatcgtggagttcaccaaggaggagtggaagctgctgacccccgcccagagaaccctgtacaaggacgtgatgctggagaactacagccacctggtgagcgtgggctaccacgtgaacaagcccaacgccgtgttcaagctgaagcagggcaaggagccctggatcctggaggtggagttcccccacagaggcttccccgaggac

SEQ ID NO: 1024 - DNA, 240 bp, synthetic construct
  source 1..240: mol_type = other DNA
gagctgctgaccttcagagatgtggccatcgagttcagccccgaggagtggaagtgcctggaccccgcccagcagaacctgtacagagatgtgatgctggagaactacagaaacctggtgagcctggccgtgtacagctactacaaccagggcatcctgcccgagcagggcatccaggacagcttcaagaaggccaccctgggcagatacggcagctgcggcctggagaacatctgcctg

SEQ ID NO: 1025 - DNA, 240 bp, synthetic construct
  source 1..240: mol_type = other DNA
agcccccagaagagcagcgccctggcccccgaggaccacggcagcagctacgagggaagcgtgagcttcagagatgtggccatcgacttcagcagagaggagtggagacacctggacctgagccagagaaacctgtacagagatgtgatgctggagacctacagccacctgctgagcgtgggctaccaggtgcccaagcccgaggtggtgatgctggagcagggcaaggagccctgggcc

SEQ ID NO: 1026 - DNA, 240 bp, synthetic construct
  source 1..240: mol_type = other DNA
aacaacagccagggcagagtgaccttcgaggacgtgaccgtgaacttcacccagggcgagtggcagagactgaaccccgagcagagaaacctgtacagagatgtgatgctggagaactacagcaacctggtgagcgtgggccagggcgagaccaccaagcccgacgtgatcctgagactggagcagggcaaggagccctggctggaggaggaggaggtgctgggcagcggcagagccgag

SEQ ID NO: 1027 - DNA, 240 bp, synthetic construct
  source 1..240: mol_type = other DNA
gccatggccctgcccatgcccggcccccaggaggccgtggtgttcgaggacgttgccgtgtacttcaccagaatcgagtggagctgcctggcccccgaccagcaggccctgtacagagatgtgatgctggagaactacggcaacctggccagcctgggcttcctggtggccaagcccgccctgatcagcctgctggagcagggcgaggagcccggagccctgatcctgcaggtggccgag

SEQ ID NO: 1028 - DNA, 240 bp, synthetic construct
  source 1..240: mol_type = other DNA
gacagcagccagcacctggtgaccttcgaggacgtggccgtggacttcacccaggaggagtggaccctgctggaccaggcccagagagacctgtacagagatgtgatgctggagaactacaagaacctgatcatcctggccggcagcgagctgttcaagagaagcctgatgagcggcctggagcagatggaggagctgagaaccggcgtgaccggcgtgctgcaggagctggacctgcag

SEQ ID NO: 1029 - DNA, 240 bp, synthetic construct
  source 1..240: mol_type = other DNA
accaccttcaaggaggccgtgaccttcaaggacgtggccgtgttcttcaccgaggaggagctgggcctgctggaccccgcccagagaaagctgtaccaggacgtgatgctggagaacttcaccaacctgctgagcgtgggccaccagcccttccaccccttccacttcctgagagaggagaagttctggatgatggagaccgccacccagagagagggcaacagcggcggcaagaccatc

SEQ ID NO: 1030 - DNA, 240 bp, synthetic construct
  source 1..240: mol_type = other DNA
ggccccctgcagttcagagatgtggccatcgagttcagcctggaggagtggcactgcctggacatggcccagagaaacctgtacagagatgtgatgctggagaactacagaaacctggtgttcctgggcatcgtggtgagcaagcccgacctgatcacccacctggagcagggcaagaagcccagcaccatgcagagacacgagatggtggccaaccccagcgtgctgtgcagccacttc

SEQ ID NO: 1031 - DNA, 240 bp, synthetic construct
  source 1..240: mol_type = other DNA
aacaagagccagggcagcgtgagcttcaccgacgtgaccgtggacttcacccaggaggagtgggagcagctggaccccagccagagaatcctgtacatggacgtgatgctggagaactacagcaacctgctgagcgtggaggtgtggaaggccgacgaccagatggagagagaccacagaaaccccgacgagcaggccagacagttcctgatcctgaagaaccagacccccatcgaggag

SEQ ID NO: 1032 - DNA, 240 bp, synthetic construct
  source 1..240: mol_type = other DNA
gaggaggaggagatgaacgacggcagccagatggtgagaagccaggagagcctgaccttccaggacgtggccgtggacttcaccagagaggagtgggaccagctgtaccccgcccagaagaacctgtacagagatgtgatgctggagaactacagaaacctggtggccctgggctaccagctgtgcaagcccgaggtgatcgcccagctggagctggaggaggagtgggtgatcgagaga

SEQ ID NO: 1033 - DNA, 240 bp, synthetic construct
  source 1..240: mol_type = other DNA
gaggaggccaccaagaagagcaaggagaaggagcccggcatggccctgccccagggcagactgaccttcagagatgtggccatcgagttcagcctggaggagtggaagtgcctgaaccccgcccagagagccctgtacagagccgtgatgctggagaactacagaaacctggagttcgtggacagcagcctgaagagcatgatggagttcagcagcaccagacacagcatcaccggcgag

SEQ ID NO: 1034 - DNA, 240 bp, synthetic construct
  source 1..240: mol_type = other DNA
gagaagacccccgccggcagaatcgtggccgactgcctgaccgactgctaccaggacagcgtgaccttcgacgacgtggccgtggacttcacccaggaggagtggaccctgctggacagcacccagagaagcctgtacagcgacgtgatgctggagaactacaagaacctggccaccgtgggcggccagatcatcaagcccagcctgatcagctggctggagcaggaggagagcagaacc

SEQ ID NO: 1035 - DNA, 240 bp, synthetic construct
  source 1..240: mol_type = other DNA
aagaccagaaagagaaagagaaaggccaaggagagcggcatggccctgctgcagggcctgctgaccttcagagatgtggccatcgagttcagccaggaggagtggaagtgcctggaccccgcccagagaaccctgtacagagatgtgatgctggagaactacagaaacctggtgagcctggacaccagcagcaagtgcatgatgaagatgttcagcagcaccggccagggcaacaccgag

SEQ ID NO: 1036 - DNA, 240 bp, synthetic construct
  source 1..240: mol_type = other DNA
gccatgagccaggagagcctgaccttcaaggacgtgttcgtggacttcaccctggaggagtggcagcagctggacagcgcccagaagaacctgtacagagatgtgatgctggagaactacagccacctggtgagcgtgggccacctggtgggcaagcccgacgtgatcttcagactgggccccggcgacgagagctggatggccgacggcggcacacctgtgagaacctgcgccggcgag

SEQ ID NO: 1037 - DNA, 240 bp, synthetic construct
  source 1..240: mol_type = other DNA
gacagcgtggccttcgaggacgtggccgtgaacttcaccctggaggagtgggccctgctggaccccagccagaagaacctgtacagagatgtgatgagagagaccttcagaaacctggccagcgtgggcaagcagtgggaggaccagaacatcgaggaccccttcaagatccccagaagaaacatcagccacatccccgagagactgtgcgagagcaaggagggcggccagggcgaggag

SEQ ID NO: 1038 - DNA, 240 bp, synthetic construct
  source 1..240: mol_type = other DNA
atgttccaggacagcgtggccttcgaggacgtggccgtgagcttcacccaggaggagtgggccctgctggaccccagccagaagaacctgtacagagatgtgatgcaggagaccttcaagaacctgaccagcgtgggcaagacctggaaggtgcagaacatcgaggacgagtacaagaaccccagaagaaacctgagcctgatgagagagaagctgtgcgagagcaaggagagccaccac

SEQ ID NO: 1039 - DNA, 240 bp, synthetic construct
  source 1..240: mol_type = other DNA
gccgtggtggccaccctgagactgagcgcccagggcaccgtgaccttcgaggacgtggccgtgaagttcacccaggaggagtggaacctgctgagcgaggcccagagatgcctgtacagagatgtgaccctggagaacctggccctgatgagcagcctgggctgctggtgcggcgtggaggacgaggccgcccctagcaagcagagcatctacatccagagagagacccaggtgagaacc

SEQ ID NO: 1040 - DNA, 240 bp, synthetic construct
  source 1..240: mol_type = other DNA
gaggaggaggaggaggacgaggacgaggacgacctgctgaccgccggctgccaggagctggtgaccttcgaggacgtggccgtgtacttcagcctggaggagtgggagagactggaggccgaccagagaggcctgtaccaggaggtgatgcaggagaactacggcatcctggtgagcctgggctaccccatccccaagcccgacctgatcttcagactggagcagggcgaggagccctgg

SEQ ID NO: 1041 - DNA, 240 bp, synthetic construct
  source 1..240: mol_type = other DNA
accaagttccaggagatggtgaccttcaaggacgtggccgtggtgttcaccagagaggagctgggcctgctggacctggcccagagaaagctgtaccaggacgtgatgctggagaacttcagaaacctgctgagcgtgggctaccagcccttcaagctggacgtgatcctgcagctgggcaaggaggacaagctgagaatgatggagaccgagatccagggcgacggctgcagcggccac

SEQ ID NO: 1042 - DNA, 240 bp, synthetic construct
  source 1..240: mol_type = other DNA
gaggaggccgcccagaagagaaagggcaaggagcccggcatggccctgccccagggcagactgaccttcagagatgtggccatcgagttcagcctggccgagtggaagtgcctgaaccccagccagagagccctgtacagagaggtgatgctggagaactacagaaacctggaggccgtggacatcagcagcaagtgcatgatgaaggaggtgctgagcaccggccagggcaacaccgag

SEQ ID NO: 1043 - DNA, 240 bp, synthetic construct
  source 1..240: mol_type = other DNA
gacaccgtggtgttcgaggacgtggtggtggacttcaccctggaggagtgggccctgctgaaccccgcccagagaaagctgtacagagatgtgatgctggagaccttcaagcacctggccagcgtggacaacgaggcccagctgaaggccagcggcagcatcagccagcaggacaccagcggcgagaagctgagcctgaagcagaagatcgagaagttcaccagaaagaacatctgggcc

SEQ ID NO: 1044 - DNA, 240 bp, synthetic construct
  source 1..240: mol_type = other DNA
accaccttcaaggagggcctgaccttcaaggacgtggccgtggtgttcaccgaggaggagctgggcctgctggaccccgtgcagagaaacctgtaccaggacgtgatgctggagaacttcagaaacctgctgagcgtgggccaccaccccttcaagcacgacgtgttcctgctggagaaggagaagaagctggacatcatgaagaccgccacccagagaaagggcaagagcgccgacaag

SEQ ID NO: 1045 - DNA, 240 bp, synthetic construct
  source 1..240: mol_type = other DNA
agcgccctgcagcaggagttctggaagatccagaccagcaacggcatccagatggacctggtgaccttcgacagcgtggccgtggagttcacccaggaggagtggaccctgctggaccccgcccagagaaacctgtacagcgacgtgatgctggagaactacaagaacctgagcagcgtgggctaccagctgttcaagcccagcctgatcagctggctggaggaggaggaggagctgagc

SEQ ID NO: 1046 - DNA, 240 bp, synthetic construct
  source 1..240: mol_type = other DNA
gactgcgtgatcttcgaggaggtggccgtgaacttcacccccgaggagtgggccctgctggaccacgcccagagaagcctgtacagagatgtgatgctggagacctgcagaaacctggccagcctggactgctacatctacgtgagaaccagcggcagcagcagccagagagatgtgttcggcaacggcatcagcaacgacgaggagatcgtgaagttcaccggcagcgacagctggagc

SEQ ID NO: 1047 - DNA, 240 bp, synthetic construct
  source 1..240: mol_type = other DNA
gagctgctgaccttcagagatgtgaccatcgagttcagcctggaggagtgggagttcctgaaccccgcccagcagagcctgtacagaaaggtgatgctggagaactacagaaacctggtgagcctgggcctgaccgtgagcaagcccgagctgatcagcagactggagcagagacaggagccctggaacgtgaagagacacgagaccatcgccaagccccccgccatgagcagccactac

SEQ ID NO: 1048 - DNA, 240 bp, synthetic construct
  source 1..240: mol_type = other DNA
atcaagacccaggagagcctgaccctggaggacgtggccgtggagttcagctgggaggagtggcagctgctggacaccgcccagaagaacctgtacagagatgtgatggtggagaactacaaccacctggtgagcctgggctaccagaccagcaagcccgacgtgctgagcaagctggcccacggccaggagccctggaccaccgacgccaagatccagaacaagaactgccccggcatc

SEQ ID NO: 1049 - DNA, 240 bp, synthetic construct
  source 1..240: mol_type = other DNA
cccgcccacgtgcccggcgaggccggccccagaagaaccagagagagcagacccggcgctgtgagcttcgccgacgtggccgtgtacttcagccccgaggagtgggagtgcctgagacccgcccagagagccctgtacagagatgtgatgagagagaccttcggccacctgggcgccctgggcttcagcgtgcccaagcccgccttcatcagctgggtggagggcgaggtggaggcctgg

SEQ ID NO: 1050 - DNA, 240 bp, synthetic construct
  source 1..240: mol_type = other DNA
ggctgccccggcgaccaggtgacccccaccagaagcctgaccgcccagctgcaggagaccatgaccttcaaggacgtggaggtgaccttcagccaggacgagtggggctggctggacagcgcccagagaaacctgtacagagatgtgatgctggagaactacagaaacatggccagcctggtgggccccttcaccaagcccgccctgatcagctggctggaggccagagagccctggggc

SEQ ID NO: 1051 - DNA, 240 bp, synthetic construct
  source 1..240: mol_type = other DNA
gccagagacctggtgatgttcagagatgtggccgtggacttcagccaggaggagtgggagtgcctgaacagctaccagagaaacctgtacagagatgtgatcctggagaactacagcaacctggtgagcctggccggctgcagcatcagcaagcccgacgtgatcaccctgctggagcagggcaaggagccctggatggtggtgagagatgagaagagaagatggaccctggacctggag

SEQ ID NO: 1052 - DNA, 240 bp, synthetic construct
  source 1..240: mol_type = other DNA
accaccctgaaggaggccgtgaccttcaaggacgtggccgtggtgttcaccgaggaggagctgagactgctggacctggcccagagaaagctgtacagagaggtgatgctggagaacttcagaaacctgctgagcgtgggccaccagagcctgcacagagacaccttccacttcctgaaggaggagaagttctggatgatggagaccgccacccagagagagggcaacctgggcggcaag

SEQ ID NO: 1053 - DNA, 240 bp, synthetic construct
  source 1..240: mol_type = other DNA
agcccccccagccccagaagcagcatggccgccgtggccctgagagacagcgcccagggcatgaccttcgaggacgtggccatctacttcagccaggaggagtgggagctgctggacgagagccagagattcctgtactgcgacgtgatgctggagaacttcgcccacgtgaccagcctgggctactgccacggcatggagaacgaggccatcgccagcgagcagagcgtgagcatccag

SEQ ID NO: 1054 - DNA, 240 bp, synthetic construct
  source 1..240: mol_type = other DNA
gacgaggaggcccagaagagaaaggccaaggagagcggcatggccctgccccagggcagactgaccttcatggacgtggccatcgagttcagccaggaggagtggaagagcctggaccccggccagagagccctgtacagagatgtgatgctggagaactacagaaacctggtgttcctgggcatctgcctgcccgacctgagcatcatcagcatgctgaagcagagaagagagcccctg

SEQ ID NO: 1055 - DNA, 240 bp, synthetic construct
  source 1..240: mol_type = other DNA
gccctgacccagggccccctgaagttcatggacgtggccatcgagttcagccaggaggagtggaagtgcctggaccccgcccagagaaccctgtacagagatgtgatgctggagaactacagaaacctggtgagcctgggcatctgcctgcccgacctgagcgtgaccagcatgctggagcagaagagagacccctggaccctgcagagcgaggagaagatcgccaacgaccccgacggc

SEQ ID NO: 1056 - DNA, 240 bp, synthetic construct
  source 1..240: mol_type = other DNA
accatgttcaaggaggccgtgaccttcaaggacgtggccgtggtgttcaccgaggaggagctgggcctgctggacgtgagccagagaaagctgtacagagatgtgatgctggagaacttcagaaacctgctgagcgtgggccaccagctgagccacagagacaccttccacttccagagagaggagaagttctggatcatggagaccgccacccagagagagggcaacagcggcggcaag

SEQ ID NO: 1057 - DNA, 240 bp, synthetic construct
  source 1..240: mol_type = other DNA
cagggcaccgtggccttcgaggacgtggccgtgaacttcagccaggaggagtggagcctgctgagcgaggtgcagagatgcctgtaccacgacgtgatgctggagaactgggtgctgatcagcagcctgggctgctggtgcggcagcgaggacgaggaggcccccagcaagaagagcatcagcatccagagagtgagccaggtgagcacccccggcgccggcgtgagccccaagaaggcc

SEQ ID NO: 1058 - DNA, 240 bp, synthetic construct
  source 1..240: mol_type = other DNA
gccgaggccttcggcgacagcgaggaggacggcgaggacgtgttcgaggtggagaagatcctggacatgaagaccgagggcggcaaggtgctgtacaaggtgagatggaagggctacaccagcgacgacgacacctgggagcccgagatccacctggaggactgcaaggaggtgctgctggagttcagaaagaagatcgccgagaacaaggccaaggccgtgagaaaggacatccagaga

SEQ ID NO: 1059 - DNA, 240 bp, synthetic construct
  source 1..240: mol_type = other DNA
gtgctgcagatgcagaacagcgagcaccacggccagagcatcaagacccagaccgacagcatcagcctggaggacgtggccgtgaacttcaccctggaggagtgggccctgctggaccccggccagagaaacatctacagagatgtgatgagagccaccttcaagaacctggcctgcatcggcgagaagtggaaggaccaggacatcgaggacgagcacaagaaccagggcagaaacctg

SEQ ID NO: 1060 - DNA, 240 bp, synthetic construct
  source 1..240: mol_type = other DNA
gccatggccctgaccgaccccgcccaggtgagcgtgaccttcgacgacgtggccgtgaccttcacccaggaggagtggggccagctggacctggcccagagaaccctgtaccaggaggtgatgctggagaactgcggcctgctggtgagcctgggctgccccgtgcccagacccgagctgatctaccacctggagcacggccaggagccctggaccagaaaggaggacctgagccagggc

SEQ ID NO: 1061 - DNA, 240 bp, synthetic construct
  source 1..240: mol_type = other DNA
gtgcacggcagcgtgaccttcagagatgtggccatcgacttcagccaggaggagtgggagtgcctgcagcccgaccagagaaccctgtacagagatgtgatgctggagaactacagccacctgatcagcctgggcagcagcatcagcaagcccgacgtgatcaccctgctggagcaggagaaggagccctggatcgtggtgagcaaggagaccagcagatggtaccccgacctggagagc

SEQ ID NO: 1062 - DNA, 240 bp, synthetic construct
  source 1..240: mol_type = other DNA
gaccccgtggcctgcgaggacgtggccgtgaacttcacccaggaggagtgggccctgctggacatcagccagagaaagctgtacagagaggtgatgctggagaccttcagaaacctgaccagcatcggcaagaagtggaaggaccagaacatcgagtacgagtaccagaaccccagaagaaacttcagaagcctgatcgagggcaacgtgaacgagatcaaggaggacagccactgcggc

SEQ ID NO: 1063 - DNA, 240 bp, synthetic construct
  source 1..240: mol_type = other DNA
atcaagttccaggagagagtgaccttcaaggacgtggccgtggtgttcaccaaggaggagctggccctgctggacaaggcccagatcaacctgtaccaggacgtgatgctggagaacttcagaaacctgatgctggtgagagatggcatcaagaacaacatcctgaacctgcaggccaagggcctgagctacctgagccaggaggtgctgcactgctggcagatctggaagcagagaatc

SEQ ID NO: 1064 - DNA, 240 bp, synthetic construct
  source 1..240: mol_type = other DNA
ggccccctgaccttcagagatgtggccatcgagttcagcctgaaggagtggcagtgcctggacaccgcccagagaaacctgtacagaaacgtgatgctggagaactacagaaacctggtgttcctgggcatcaccgtgagcaagcccgacctgatcacctgcctggagcagggcaaggaggcctggagcatgaagagacacgagatcatggtggccaagcccaccgtgatgtgcagccac

SEQ ID NO: 1065 - DNA, 240 bp, synthetic construct
  source 1..240: mol_type = other DNA
accatgagcaaggaggccgtgaccttcaaggacgtggccgtggtgttcaccgaggaggagctgggcctgctggacctggcccagagaaagctgtacagagatgtgatgctggagaacttcagaaacctgctgagcgtgggccaccagcccttccacagagacaccttccacttcctgagagaggagaagttctggatgatggacatcgccacccagagagagggcaacagcggcggcaag

SEQ ID NO: 1066 - DNA, 240 bp, synthetic construct
  source 1..240: mol_type = other DNA
ggccccctggagttcagagatgtggccatcgagttcagcctggaggagtggcactgcctggacaccgcccagcagaacctgtacagagatgtgatgctggagaactacagacacctggtgttcctgggcatcgtggtgaccaagcccgacctgatcacctgcctggagcagggcaagaagcccttcaccgtgaagagacacgagatgatcgccaagagccccgtgatgtgcttccacttc

SEQ ID NO: 1067 - DNA, 240 bp, synthetic construct
  source 1..240: mol_type = other DNA
ggccacaccgagggcggcgagctggtgaacgagctgctgaagagctggctgaagggcctggtgaccttcgaggacgtggccgtggagttcacccaggaggagtgggccctgctggaccccgcccagagaaccctgtacagagatgtgatgctggagaactgcagaaacctggccagcctgggcaaccaggtggacaagcccagactgatcagccagctggagcaggaggacaaggtgatg

SEQ ID NO: 1068 - DNA, 240 bp, synthetic construct
  source 1..240: mol_type = other DNA
gccgagcccgccagcgtgaccgtgaccttcgacgacgtggccctgtacttcagcgagcaggagtgggagatcctggagaagtggcagaagcagatgtacaagcaggagatgaagaccaactacgagaccctggacagcctgggctacgccttcagcaagcccgacctgatcacctggatggagcagggcagaatgctgctgatcagcgagcagggctgcctggacaagaccagaagaacc

SEQ ID NO: 1069 - DNA, 240 bp, synthetic construct
  source 1..240: mol_type = other DNA
cacagccaggccagcgccatcagccaggacagagaggagaagatcatgagccaggagcccctgagcttcaaggacgtggccgtggtgttcaccgaggaggagctggagctgctggacagcacccagagacagctgtaccaggacgtgatgcaggagaacttcagaaacctgctgagcgtgggcgagagaaaccccctgggcgacaagaacggcaaggacaccgagtacatccaggacgag

SEQ ID NO: 1070 - DNA, 240 bp, synthetic construct
  source 1..240: mol_type = other DNA
ggcagcctggaggagggcagaagagccaccggcctgcccgccgcccaagtgcaggagcctgtgaccttcaaggacgtggccgtggacttcacccaggaggagtggggccagctggacctggtgcagagaaccctgtacagagatgtgatgctggagacctacggccacctgctgagcgtgggcaaccagatcgccaagcccgaggtgatcagcctgctggagcagggcgaggagccctgg

SEQ ID NO: 1071 - DNA, 240 bp, synthetic construct
  source 1..240: mol_type = other DNA
accaccttcaaggaggccgtgaccttcaaggacgtggccgtggtgttcaccgaggaggagctgggcctgctggaccccgcccagagaaagctgtacagagatgtgatgctggagaacttcagaaacctgctgagcgtgggccaccagcccttccaccaggacacctgccacttcctgagagaggagaagttctggatgatgggcaccgccacccagagagagggcaacagcggcggcaag

SEQ ID NO: 1072 - DNA, 240 bp, synthetic construct
  source 1..240: mol_type = other DNA
gccaagctgtacgaggccgtgaccttcaaggacgtggccgtgatcttcaccgaggaggagctgggcctgctggaccccgcccagagaaagctgtacagagatgtgatgctggagaacttcagaaacctgctgagcgtgggcggcaagatccagaccgagatggagaccgtgcccgaggccggcacccacgaggagttcagctgcaagcagatctgggagcagatcgccagcgacctgacc

SEQ ID NO: 1073 - DNA, 240 bp, synthetic construct
  source 1..240: mol_type = other DNA
agaagcgacctgttcctgcccgacagccagaccaacgaggagagaaagcagtacgacagcgtggccttcgaggacgtggccgtgaacttcacccaggaggagtgggccctgctgggccccagccagaagagcctgtacagagatgtgatgtgggagaccatcagaaacctggactgcatcggcatgaagtgggaggacaccaacatcgaggaccagcacagaaaccccagaagaagcctg

SEQ ID NO: 1074 - DNA, 240 bp, synthetic construct
  source 1..240: mol_type = other DNA
cccggcacccccggcagcctggagatgggcctgctgaccttcagagatgtggccatcgagttcagccccgaggagtggcagtgcctggacaccgcccagcagaacctgtacagaaacgtgatgctggagaactacagaaacctggccttcctgggcatcgccctgagcaagcccgacctgatcacctacctggagcagggcaaggagccctggaacatgaagcagcacgagatggtggac

SEQ ID NO: 1075 - DNA, 240 bp, synthetic construct
  source 1..240: mol_type = other DNA
acccccggcgtgagagtgagcaccgaccccgagcaggtgaccttcgaggacgtggtggtgggcttcagccaggaggagtggggccagctgaagcccgcccagagaaccctgtacagagatgtgatgctggacaccttcagactgctggtgagcgtgggccactggctgcccaagcccaacgtgatcagcctgctggagcaggaggccgagctgtgggccgtggagagcagactgccccag

SEQ ID NO: 1076 - DNA, 240 bp, synthetic construct
  source 1..240: mol_type = other DNA
gagcagacccaggccgccggcatggtggccggctggctgatcaactgctaccaggacgccgtgaccttcgacgacgtggccgtggacttcacccaggaggagtggaccctgctggaccccagccagagagacctgtacagagatgtgatgctggagaactacgagaacctggccagcgtggagtggagactgaagaccaagggccccgccctgagacaggacagaagctggttcagagcc

SEQ ID NO: 1077 - DNA, 240 bp, synthetic construct
  source 1..240: mol_type = other DNA
cccagcgaggccaacagcatccagagcgccaacgccaccaccaagaccagcgagaccaaccacaccagcagacccagactgaagaacgtggacagaagcaccgcccagcagctggccgtgaccgtgggcaacgtgaccgtgatcatcaccgacttcaaggagaagaccagaagcagcagcaccagcagcagcaccgtgaccagcagcgccggcagcgagcagcagaaccagagcagcagc

SEQ ID NO: 1078 - DNA, 240 bp, synthetic construct
  source 1..240: mol_type = other DNA
gccctgacccagggccagctgagcttcagcgacgtggccatcgagttcagccaggaggagtggaagtgcctggaccccggccagaaggccctgtacagagatgtgatgctggagaactacagaaacctggtgagcctgggcgaggacaacgtgagacccgaggcctgcatctgcagcggcatctgcctgcccgacctgagcgtgaccagcatgctggagcagaagagagacccctggacc

SEQ ID NO: 1079 - DNA, 240 bp, synthetic construct
  source 1..240: mol_type = other DNA
gccgccgccgccgccctgagagcccccgcccagagcagcgttacattcgaggacgtggccgtgaacttcagcctggaggagtggagcctgctgaacgaggcccagagatgcctgtacagagatgtgatgctggagaccctgaccctgatcagcagcctgggctgctggcacggcggcgaggacgaggccgctcctagcaagcagagcacctgcatccacatctacaaggaccagggcggc

SEQ ID NO: 1080 - DNA, 240 bp, synthetic construct
  source 1..240: mol_type = other DNA
gcccagggcagcgtgagcttcaacgacgtgaccgtggacttcacccaggaggagtggcagcacctggaccacgcccagaagaccctgtacatggacgtgatgctggagaactactgccacctgatcagcgtgggctgccacatgaccaagcccgacgtgatcctgaagctggagagaggcgaggagccctggaccagcttcgccggccacacctgcctggaggagaactggaaggccgag

SEQ ID NO: 1081 - DNA, 240 bp, synthetic construct
  source 1..240: mol_type = other DNA
gaccccgtggccttcaaggacgtggccgtgaacttcacccaggaggagtgggccctgctggacatcagccagagaaagctgtacagagaggtgatgctggagaccttcagaaacctgaccagcctgggcaagagatggaaggaccagaacatcgagtacgagcaccagaaccccagaagaaacttcagaagcctgatcgaggagaaggtgaacgagatcaaggacgacagccactgcggc

SEQ ID NO: 1082 - DNA, 240 bp, synthetic construct
  source 1..240: mol_type = other DNA
agcaaggacctggtgaccttcggcgacgtggccgtgaacttcagccaggaggagtgggagtggctgaaccccgcccagagaaacctgtacagaaaggtgatgctggagaactacagaagcctggtgagcctgggcgtgagcgtgagcaagcccgacgtgatcagcctgctggagcagggcaaggagccctggatggtgaagaaggagggcaccagaggcccctgccccgactgggagtac

SEQ ID NO: 1083 - DNA, 240 bp, synthetic construct
  source 1..240: mol_type = other DNA
gacagcgtggccttcgaggacgtggccatcaacttcacctgcgaggagtgggccctgctgggccccagccagaagagcctgtacagagatgtgatgcaggagaccatcagaaacctggactgcatcggcatgatctggcagaaccacgacatcgaggaggaccagtacaaggacctgagaagaaacctgagatgccacatggtggagagagcctgcgagatcaaggacaacagccagtgc

SEQ ID NO: 1084 - DNA, 240 bp, synthetic construct
  source 1..240: mol_type = other DNA
ggccccctgaccttcatggacgtggccatcgagttctgcctggaggagtggcagtgcctggacatcgcccagcagaacctgtacagaaacgtgatgctggagaactacagaaacctggtgttcctgggcatcgccgtgagcaagcccgacctgatcacctgcctggagcaggagaaggagccctgggagcccatgagaagacacgagatggtggccaagccccccgtgatgtgcagccac

SEQ ID NO: 1085 - DNA, 240 bp, synthetic construct
  source 1..240: mol_type = other DNA
cagagcaacgacatcgccagaggcttcgagagaggcctggagcccgagaagatcatcggcgccaccgacagctgcggcgacctgatgttcctgatgaagtggaaggacaccgacgaggccgacctggtgctggccaaggaggccaacgtgaagtgcccccagatcgtgatcgccttctacgaggagagactgacctggcacgcctaccccgaggacgccgagaacaaggagaaggagacc

SEQ ID NO: 1086 - DNA, 240 bp, synthetic construct
  source 1..240: mol_type = other DNA
gccctgcccgccaaggacagcgcctggccctgggaggagaagcccagatacctgggccccgtgaccttcgaggacgtggccgtgctgttcaccgaggccgagtggaagagactgagcctggagcagagaaacctgtacaaggaggtgatgctggagaacctgagaaacctggtgagcctggccgagagcaagcccgaggtgcacacctgccccagctgccccctggccttcggcagccag

SEQ ID NO: 1087 - DNA, 240 bp, synthetic construct
  source 1..240: mol_type = other DNA
gacgccaagagcctgaccgcctggagcagaaccctggtgaccttcaaggacgtgttcgtggacttcaccagagaggagtggaagctgctggacaccgcccagcagatcgtgtacagaaacgtgatgctggagaactacaagaacctggtgagcctgggctaccagctgaccaagcccgacgtgatcctgagactggagaagggcgaggagccctggctggtggagagagagatccaccag

SEQ ID NO: 1088 - DNA, 240 bp, synthetic construct
  source 1..240: mol_type = other DNA
gacgccgtggccttcgaggacgtggccgtgaacttcacccaggaggagtgggccctgctgggccccagccagaagaacctgtacagatacgtgatgcaggagaccatcagaaacctggactgcatcagaatgatctgggaggagcagaacaccgaggaccagtacaagaaccccagaagaaacctgagatgccacatggtggagagattcagcgagagcaaggacagcagccagtgcggc

SEQ ID NO: 1089 - DNA, 240 bp, synthetic construct
  source 1..240: mol_type = other DNA
gagaagaccaaggtggagagaatggtggaggactacctggccagcggctaccaggacagcgtgaccttcgacgacgtggccgtggacttcacccccgaggagtgggccctgctggacaccaccgagaagtacctgtacagagatgtgatgctggagaactacatgaacctggccagcgtggagtgggagatccagcccagaaccaagagaagcagcctgcagcagggcttcctgaagaac

SEQ ID NO: 1090 - DNA, 240 bp, synthetic construct
  source 1..240: mol_type = other DNA
gacagcgtggccttcgaggacgtggacgtgaacttcacccaggaggagtgggccctgctggaccccagccagaagaacctgtacagagatgtgatgtgggagaccatgagaaacctggccagcatcggcaagaagtggaaggaccagaacatcaaggaccactacaagcacagaggcagaaacctgagaagccacatgctggagagactgtaccagaccaaggacggcagccagagaggc

SEQ ID NO: 1091 - DNA, 240 bp, synthetic construct
  source 1..240: mol_type = other DNA
atcgagagccaggagcccgtgaccttcgaggacgtggccgtggacttcacccaggaggagtggcagcagctgaaccccgcccagaagaccctgcacagagatgtgatgctggagacctacaaccacctggtgagcgtgggctgcagcggcatcaagcccgacgtgatcttcaagctggagcacggcaaggacccctggatcatcgagagcgagctgagcagatggatctaccccgacaga

SEQ ID NO: 1092 - DNA, 240 bp, synthetic construct
  source 1..240: mol_type = other DNA
gccgtgggcctgtgcaaggccatgagccagggcctggtgaccttcagagatgtggccctggacttcagccaggaggagtgggagtggctgaagcccagccagaaggacctgtacagagatgtgatgctggagaactacagaaacctggtgtggctgggcctgagcatcagcaagcccaacatgatcagcctgctggagcagggcaaggagccctggatggtggagagaaagatgagccag

SEQ ID NO: 1093 - DNA, 240 bp, synthetic construct
  source 1..240: mol_type = other DNA
gacaaggtggaggaggaggccatggcccccggcctgcccaccgcttgcagccaagagcctgtgaccttcgccgacgtggccgtggtgttcacccccgaggagtgggtgttcctggacagcacccagagaagcctgtacagagatgtgatgctggagaactacagaaacctggccagcgtggccgaccagctgtgcaagcccaacgccctgagctacctggaggagagaggcgagcagtgg

SEQ ID NO: 1094 - DNA, 240 bp, synthetic construct
  source 1..240: mol_type = other DNA
accttcgaggacgtggccgtgtacttcagccaggaggagtggggcctgctggacaccgcccagagagccctgtacagacacgtgatgctggagaacttcaccctggtgaccagcctgggcctgagcaccagcagacccagagtggtgatccagctggagagaggcgaggagccctgggtgcccagcggcaaggacatgaccctggccagaaacacctacggcagactgaacagcggcagc

SEQ ID NO: 1095 - DNA, 240 bp, synthetic construct
  source 1..240: mol_type = other DNA
gccgagccccccagactgcccctgaccttcgaggacgtggccatctacttcagcgagcaggagtggcaggacctggaggcctggcagaaggagctgtacaagcacgtgatgagaagcaactacgagaccctggtgagcctggacgacggcctgcccaagcccgagctgatcagctggatcgagcacggcggcgagcccttcagaaagtggagagagagccagaagagcggcaacatcatc

SEQ ID NO: 1096 - DNA, 240 bp, synthetic construct
  source 1..240: mol_type = other DNA
gacagcgtggtgttcgaggacgtggccgtgaacttcacccaggaggagtgggccctgctgggccccagccagaagaagctgtacagagatgtgatgcaggagaccttcgtgaacctggccagcatcggcgagaactgggaggagaagaacatcgaggaccacaagaaccagggcagaaagctgagaagccacatggtggagagactgtgcgagagaaaggagggcagccagttcggcgag

SEQ ID NO: 1097 - DNA, 240 bp, synthetic construct
  source 1..240: mol_type = other DNA
gccgccgccgccctgagagaccccgcccagggctgcgtgaccttcgaagacgtgaccatctacttcagccaggaggagtgggtgctgctggacgaggcccagagactgctgtactgcgacgtgatgctggagaacttcgccctgatcgccagcctgggcctgatcagcttcagaagccacatcgtgagccagctggagatgggcaaggagccctgggtgcccgacagcgtggacatgacc

SEQ ID NO: 1098 - DNA, 240 bp, synthetic construct
  source 1..240: mol_type = other DNA
gccgcccccgccctggccctggtgagcttcgaggacgtggtggtgaccttcaccggcgaggagtggggccacctggacctggcccagagaaccctgtaccaggaggtgatgctggagacctgcagactgctggtgagcctgggccaccccgtgcccaagcccgagctgatctacctgctggagcacggccaggagctgtggaccgtgaagagaggcctgagccagagcacctgcgccggc

SEQ ID NO: 1099 - DNA, 240 bp, synthetic construct
  source 1..240: mol_type = other DNA
atcaagagccaggagagcctgaccctggaggacgtggccgtggagttcacctgggaggagtggcagctgctgggccccgcccagaaggacctgtacagagatgtgatgctggagaactacagcaacctggtgagcgtgggctaccaggccagcaagcccgacgccctgttcaagctggagcagggcgagccctggaccgtggagaacgagatccacagccagatctgccccgagatcaag

SEQ ID NO: 1100 - DNA, 240 bp, synthetic construct
  source 1..240: mol_type = other DNA
ggcgagagcctggagagcagagtgaccctgggcagcctgaccgccgagagccaggagctgctgaccttcaaggacgtgagcgtggacttcacccaggaggagtggggccagctggcccccgcccacagaaacctgtacagagaggtgatgctggagaactacggcaacctggtgagcgtgggctgccagctgagcaagcccggcgtgatcagccagctggagaagggcgaggagccctgg

SEQ ID NO: 1101 - DNA, 240 bp, synthetic construct
  source 1..240: mol_type = other DNA
gccagcgtggccctggaggacgtggccgtgaacttcaccagagaggagtgggccctgctgggcccctgccagaagaacctgtacaaggacgtgatgcaggagaccatcagaaacctggactgcgtgggcatgaagtggaaggaccagaacatcgaggaccagtacagataccccagaaagaacctgagatgcagaatgctggagagattcgtggagagcaaggacggcacccagtgcggc

SEQ ID NO: 1102 - DNA, 240 bp, synthetic construct
  source 1..240: mol_type = other DNA
accgagagccagggcaccgtgaccttcaaggacgtggccatcgacttcacccaggaggagtggaagagactggaccccgcccagagaaagctgtacagaaacgtgatgctggagaactacaacaacctgatcaccgtgggctaccccttcaccaagcccgacgtgatcttcaagctggagcaggaggaggagccctgggtgatggaggaggaggtgctgagaagacactggcagggcgag

SEQ ID NO: 1103 - DNA, 240 bp, synthetic construct
  source 1..240: mol_type = other DNA
gacagcgtggccagcgaggacgtggccgtgaacttcaccctggaggagtgggccctgctggaccccagccagaagaagctgtacagagatgtgatgagagagaccttcagaaacctggcctgcgtgggcaagaagtgggaggaccagagcatcgaggactggtacaagaaccagggcagaatcctgagaaaccacatggaggagggcctgagcgagagcaaggagtacgaccagtgcggc

SEQ ID NO: 1104 - DNA, 240 bp, synthetic construct
  source 1..240: mol_type = other DNA
gaggagacccagggcgagctgaccagcagctgcggcagcaagaccatggccaacgtgagcctggccttcagagatgtgagcatcgacctgagccaggaggagtgggagtgcctggacgccgtgcagagagacctgtacaaggacgtgatgctggagaactacagcaacctggtgagcctgggctacaccatccccaagcccgacgtgatcaccctgctggagcaggagaaggagccctgg

SEQ ID NO: 1105 - DNA, 240 bp, synthetic construct
  source 1..240: mol_type = other DNA
gccgccatcctgctgaccaccagacccaaggtgcccgtgagcttcgaggacgtgagcgtgtacttcaccaagaccgagtggaagctgctggacctgagacagaaggtgctgtacaagagagtgatgctggagaactacagccacctggtgagcctgggcttcagcttcagcaagccccacctgatcagccagctggagagaggcgagggcccctgggtggccgacatccccagaacctgg

SEQ ID NO: 1106 - DNA, 240 bp, synthetic construct
  source 1..240: mol_type = other DNA
aaggacaaggtggagaaggagaagagcgagaaggagaccaccagcaagaagaacagccacaagaagaccagacccagactgaagaacgtggacagaagcagcgcccagcacctggaggtgaccgtgggcgacctgaccgtgatcatcaccgacttcaaggagaagaccaagagcccccccgccagcagcgccgccagcgctgaccaacacagccaaagcggaagcagcagcgacaacacc

SEQ ID NO: 1107 - DNA, 240 bp, synthetic construct
  source 1..240: mol_type = other DNA
ggccccctgaccttcaccgacgtggccatcaagttcagcctggaggagtggcagttcctggacaccgcccagcagaacctgtacagagatgtgatgctggagaactacagaaacctggtgttcctgggcgtgggcgtgagcaagcccgacctgatcacctgcctggagcagggcaaggagccctggaacatgaagagacacaagatggtggccaagccccccgtggtgtgcagccacttc

SEQ ID NO: 1108 - DNA, 240 bp, synthetic construct
  source 1..240: mol_type = other DNA
agaaagcccaacccccaggccatggccgccctgttcctgagcgccccccctcaagccgaagtgaccttcgaggacgtggccgtgtacctgagcagagaggagtggggcagactgggccccgcccagagaggcctgtacagagatgtgatgctggagacctacggcaacctggtgagcctgggcgtgggccccgccggacccaaacctggagtgatcagccagctggagagaggcgacgag

SEQ ID NO: 1109 - DNA, 240 bp, synthetic construct
  source 1..240: mol_type = other DNA
ctgagcctgagccccatcctgctgtacacctgcgagatgttccaggaccccgtggccttcaaggacgtggccgtgaacttcacccaggaggagtgggccctgctggacatcagccagaagaacctgtacagagaggtgatgctggagaccttctggaacctgaccagcatcggcaagaagtggaaggaccagaacatcgagtacgagtaccagaaccccagaagaaacttcagaagcgtg

SEQ ID NO: 1110 - DNA, 240 bp, synthetic construct
  source 1..240: mol_type = other DNA
gccgccggcgagcccagaagcctgctgttcttccagaagcccgtgaccttcgaggacgtggccgtgaacttcacccaggaggagtgggactgcctggacgccagccagagagtgctgtaccaggacgtgatgagcgagaccttcaagaacctgaccagcgtggccagaatcttcctgcacaagcccgagctgatcaccaagctggagcaggaggaggagcagtggagagagaccagagtg

SEQ ID NO: 1111 - DNA, 240 bp, synthetic construct
  source 1..240: mol_type = other DNA
gccgccatgcccctgaaggcccagtaccaggagatggtgaccttcgaggacgtggccgtgcacttcaccaagaccgagtggaccggcctgagccccgcccagagagccctgtacagaagcgtgatgctggagaacttcggcaacctgaccgccctgggctaccccgtgcccaagcccgccctgatcagcctgctggagagaggcgacatggcctggggcctggaggcccaggacgacccc

SEQ ID NO: 1112 - DNA, 240 bp, synthetic construct
  source 1..240: mol_type = other DNA
gccagagtgcccctgaccttcagcgacgtggccatcgacttcagccaggaggagtgggagtacctgaacagcgaccagagagacctgtacagagatgtgatgctggagaactacaccaacctggtgagcctggacttcaacttcaccaccgagagcaacaagctgagcagcgagaagagaaactacgaggtgaacgcctaccaccaggagacctggaagagaaacaagaccttcaacctg

SEQ ID NO: 1113 - DNA, 240 bp, synthetic construct
  source 1..240: mol_type = other DNA
gccagcagactgcccaccgcctggagctgcgagcccgtgaccttcgaggacgtgaccctgggcttcacccccgaggagtggggcctgctggacctgaagcagaagagcctgtacagagaggtgatgctggagaactacagaaacctggtgagcgtggagcaccagctgagcaagcccgacgtggtgagccagctggaggaggccgaggacttctggcccgtggagagaggcatcccccag

SEQ ID NO: 1114 - DNA, 240 bp, synthetic construct
  source 1..240: mol_type = other DNA
agcaagaagaagagagatgccgccgacaagcccagaggcttcgccagaggcctggaccccgagagaatcatcggcgccaccgacagcagcggcgagctgatgttcctgatgaagtggaaggacagcgacgaggccgacctggtgctggccaaggaggccaacatgaagtgcccccagatcgtgatcgccttctacgaggagagactgacctggcacagctgccccgaggacgaggcccag

SEQ ID NO: 1115 - DNA, 240 bp, synthetic construct
  source 1..240: mol_type = other DNA
gcccacaagtacgtgggcctgcagtaccacggcagcgtgaccttcgaggacgtggccatcgccttcagccagcaggagtgggagagcctggacagcagccagagaggcctgtacagagatgtgatgctggagaactacagaaacctggtgagcatgggccacagcagaagcaagccccacgtgatcgccctgctggagcagtggaaggagcccgaggtgaccgtgagaaaggacggcaga

SEQ ID NO: 1116 - DNA, 240 bp, synthetic construct
  source 1..240: mol_type = other DNA
gccgccgccagactgctgcccgtgcccgccggcccccaacctcttagcttccaggccaagctgaccttcgaggacgtggccgtgctgctgagccaggacgagtgggacagactgtgccccgcccagagaggcctgtacagaaacgtgatgatggagacctacggcaacgtggtgagcctgggcctgcccggcagcaagcccgacatcatcagccagctggagagaggcgaggacccctgg

SEQ ID NO: 1117 - DNA, 240 bp, synthetic construct
  source 1..240: mol_type = other DNA
gccgtgggcctgctgaaggccatgtaccaggagctggtgaccttcagagatgtggccgtggacttcagccaggaggagtgggactgcctggacagcagccagagacacctgtacagcaacgtgatgctggagaactacagaatcctggtgagcctgggcctgtgcttcagcaagcccagcgtgatcctgctgctggagcagggcaaggccccctggatggtgaagagagagctgaccaag

SEQ ID NO: 1118 - DNA, 240 bp, synthetic construct
  source 1..240: mol_type = other DNA
ggcctgctgaccttcagagatgtggccatcgagttcagcctggaggagtggcagtgcctggacaccgcccagagaaacctgtacaagaacgtgatcctggagaactacagaaacctggtgttcctgggcatcgccgtgagcaagcaggacctgatcacctgcctggagcaggagaaggagcccctgaccgtgaagagacacgagatggtgaacgagccccccgtgatgtgcagccacttc

SEQ ID NO: 1119 - DNA, 240 bp, synthetic construct
  source 1..240: mol_type = other DNA
ggcctgctggccttcagagatgtggccctggagttcagccccgaggagtgggagtgcctggaccccgcccagagaagcctgtacagagatgtgatgctggagaactacagaaacctgatcagcctgggcgaggacagcttcaacatgcagttcctgttccacagcctggccatgagcaagcccgagctgatcatctgcctggaggccagaaaggagccctggaacgtgaacaccgagaag

SEQ ID NO: 1120 - DNA, 240 bp, synthetic construct
  source 1..240: mol_type = other DNA
aacctgaccgagggcagagtggtgttcgaggacgtggccatctacttcagccaggaggagtggggccacctggacgaggcccagagactgctgtacagagatgtgatgctggagaacctggccctgctgagcagcctgggcagctggcacggcgccgaggacgaagaggcccctagccagcagggcttcagcgtgggcgtgagcgaggtgaccgccagcaagccctgcctgagcagccag

SEQ ID NO: 1121 - DNA, 240 bp, synthetic construct
  source 1..240: mol_type = other DNA
agccagaactacgacctgccccagaagaagcaggagaagatgaccaagttccaggaggccgtgaccttcaaggacgtggccgtggtgttcagcagagaggagctgagactgctggacctgacccagagaaagctgtacagagatgtgatggtggagaacttcaagaacctggtggccgtgggccacctgcccttccagcccgacatggtgagccagctggaggccgaggagaagctgtgg

SEQ ID NO: 1122 - DNA, 240 bp, synthetic construct
  source 1..240: mol_type = other DNA
ggccccgcccagcacaccagctggccctgcggcagcgccgttcctacactgaagagcatggtgaccttcgaggacgtggccgtgtacttcagccaggaggagtgggagctgctggacgccgcccagagacacctgtaccacagcgtgatgctggagaacctggagctggtgaccagcctgggcagctggcacggcgtggagggcgagggagctcaccccaagcagaacgtgagcgtggag

SEQ ID NO: 1123 - DNA, 240 bp, synthetic construct
  source 1..240: mol_type = other DNA
agcggctaccccggcgccgagagaaacctgctggagtacagctacttcgagaagggccccctgaccttcagagatgtggtgatcgagttcagccaggaggagtggcagtgcctggacaccgcccagcaggacctgtacagaaaggtgatgctggagaacttcagaaacctggtgttcctgggcatcgacgtgagcaagcccgacctgatcacctgcctggagcagggcaaggacccctgg

SEQ ID NO: 1124 - DNA, 240 bp, synthetic construct
  source 1..240: mol_type = other DNA
gccagaaagctggtgatgttcagagatgtggccatcgacttcagccaggaggagtgggagtgcctggacagcgcccagagagacctgtacagagatgtgatgctggagaactacagcaacctggtgagcctggacctgcccagcagatgcgccagcaaggacctgagccccgagaagaacacctacgagaccgagctgagccagtgggagatgagcgacagactggagaactgcgacctg

SEQ ID NO: 1125 - DNA, 240 bp, synthetic construct
  source 1..240: mol_type = other DNA
accttcgaggacgtggccgtggagttcagccagtgggagtggggccagctgaaccccgcccagaaggacctgtacagagaggtgatgctggagaacttcagaaacctggccatcctgggcctgctggtgagcaagccctacgtgatctgccagctggaggagggcggcgagcccttcatggtggagagagagatcagcaccggcgcccacagcgactggaagagaagaagcaagagcaag

SEQ ID NO: 1126 - DNA, 240 bp, synthetic construct
  source 1..240: mol_type = other DNA
ggccccctgcagttcagagatgtggccatcgagttcagcctggaggagtggcactgcctggacaccgcccagagaaacctgtacagaaacgtgatgctggagaactacagcaacctggtgttcctgggcatcaccgtgagcaagcccgacctgatcacctgcctggagcagggcagaaagcccctgaccatgaagagaaacgagatgatcgccaagcccagcgtgatgtgcagccacttc

SEQ ID NO: 1127 - DNA, 240 bp, synthetic construct
  source 1..240: mol_type = other DNA
atcaagttccaggagagagtgaccttcaaggacatcgccgtgatcttcaccaaggaggagctggccgtgctggacaaggcccagatcaacctgtaccaggacgtgatgctggagaacttcagaaacttcatcagcgtggacggcatcaagaacaacatcctgaacctgcagggcaagggcctgatctacctgagccaggaggagctgcactgctggaagatctggaagcagagaatcaga

SEQ ID NO: 1128 - DNA, 240 bp, synthetic construct
  source 1..240: mol_type = other DNA
gtggccggctggctgaccaactacagccaggacagcgtgaccttcgaggacgtggccgtggacttcacccaggaggagtggaccctgctggaccagacccagagaaacctgtacagagatgtgatgctggagaactacaagaacctggtggccgtggactgggagagccacatcaacaccaagtggagcgccccccagcagaacttcctgcagggcaagaccagcagcgtggtggagatg

SEQ ID NO: 1129 - DNA, 240 bp, synthetic construct
  source 1..240: mol_type = other DNA
gccgccgcctggatggcccccgcccaggagagcgtgaccttcgaggacgtggccgtgaccttcacccaggaggagtggggccagctggacgtgacccagagagccctgtacgtggaggtgatgctggagacctgcggcctgctggtggccctgggcgacagcaccaagcccgagaccgtggagcccatccccagccacctggccctgcccgaggaagtgagcctgcaggagcagctggcc

SEQ ID NO: 1130 - DNA, 240 bp, synthetic construct
  source 1..240: mol_type = other DNA
gtgctggagtggctgttcatcagccaggagcagcccaagatcaccaagagctggggccccctgagcttcatggacgtgttcgtggacttcacctgggaggagtggcagctgctggaccccgcccagaagtgcctgtacagaagcgtgatgctggagaactacagcaacctggtgagcctgggctaccagcacaccaagcccgacatcatcttcaagctggagcagggcgaggagctgtgc

SEQ ID NO: 1131 - DNA, 240 bp, synthetic construct
  source 1..240: mol_type = other DNA
gccgccgccgtgctgatggacagagtgcagagctgcgtgaccttcgaggacgtgttcgtgtacttcagcagagaggagtgggagctgctggaggaggcccagagattcctgtacagagatgtgatgctggagaacttcgccctggtggccaccctgggcttctggtgcgaggccgagcacgaggctcccagcgagcagagcgtgagcgtggagggcgtgagccaggtgagaaccgccgag

SEQ ID NO: 1132 - DNA, 240 bp, synthetic construct
  source 1..240: mol_type = other DNA
gccggcagccagttccccgacttcaagcacctgggcaccttcctggtgttcgaggagctggtgaccttcgaggacgtgctggtggacttcagccccgaggagctgagcagcctgagcgccgcccagagaaacctgtacagagaggtgatgctggagaactacagaaacctggtgagcctgggccaccagttcagcaagcccgacatcatcagcagactggaggaggaggagagctacgcc

SEQ ID NO: 1133 - DNA, 240 bp, synthetic construct
  source 1..240: mol_type = other DNA
atccagagccagatcagcttcgaggacgtggccgtggacttcaccctggaggagtggcagctgctgaaccccacccagaagaacctgtacagagatgtgatgctggagaactacagcaacctggtgttcctggaggtgtggctggacaaccccaagatgtggctgagagacaaccaggacaacctgaagagcatggagagaggccacaagtacgacgtgttcggcaagatcttcaacagc

SEQ ID NO: 1134 - DNA, 240 bp, synthetic construct
  source 1..240: mol_type = other DNA
gacctggtggccttcgaggacgtggccgtgaacttcacccaggaggagtggagcctgctggaccccagccagaagaacctgtacagagaggtgatgcaggagaccctgagaaacctggccagcatcggcgagaagtggaaggaccagaacatcgaggaccagtacaagaaccccagaaacaacctgagaagcctgctgggcgagagagtggacgagaacaccgaggagaaccactgcggc

SEQ ID NO: 1135 - DNA, 240 bp, synthetic construct
  source 1..240: mol_type = other DNA
aaggacgaggacatcaagctgagagtgatcggccaggacagcagcgagatccacttcaaggtgaagatgaccacccccctgaagaagctgaagaagagctactgccagagacagggcgtgcccgtgaacagcctgagattcctgttcgagggccagagaatcgccgacaaccacacccccgaggagctgggcatggaggaggaggacgtgatcgaggtgtaccaggagcagatcggcggc

SEQ ID NO: 1136 - DNA, 240 bp, synthetic construct
  source 1..240: mol_type = other DNA
gacagcgtggccttcgaggacgtggccgtgaacttcacccaggaggagtgggccctgctgagccccagccagaagaacctgtacagagatgtgaccctggagaccttcagaaacctggccagcgtgggcatccagtggaaggaccaggacatcgagaacctgtaccagaacctgggcatcaagctgagaagcctggtggagagactgtgcggcagaaaggagggcaacgagcacagagag

SEQ ID NO: 1137 - DNA, 240 bp, synthetic construct
  source 1..240: mol_type = other DNA
agaaacttctggatcctgagactgccccccggcagcaagggcgaggcccccaaggtgcccgtgacattcgacgacgtggccgtgtacttcagcgagctggagtggggcaagctggaggactggcagaaggagctgtacaagcacgtgatgagaggcaactacgagaccctggtgagcctggactacgccatcagcaagcccgacatcctgaccagaatcgagagaggcgaggagccctgc

SEQ ID NO: 1138 - DNA, 240 bp, synthetic construct
  source 1..240: mol_type = other DNA
gccgccgccgcccccagaagacccacccagcagggcaccgtgaccttcgaggacgtggccgtgaacttcagccaggaggagtggtgcctgctgagcgaggcccagagatgcctgtacagagatgtgatgctggagaacctggccctgatcagcagcctgggctgctggtgcggcagcaaggacgaggaggccccctgcaagcagagaatcagcgtgcagagagagagccagagcagaacc

SEQ ID NO: 1139 - DNA, 240 bp, synthetic construct
  source 1..240: mol_type = other DNA
agcggcgaggacagcggcagcttctacagctggcagaaggccaagagagagcagggcctggtgaccttcgaggacgtggccgtggacttcacccaggaggagtggcagtacctgaaccccccccagagaaccctgtacagagatgtgatgctggagacctacagcaacctggtgttcgtgggccagcaggtgaccaagcccaacctgatcctgaagctggaggtggaggagtgccccgcc

SEQ ID NO: 1140 - DNA, 240 bp, synthetic construct
  source 1..240: mol_type = other DNA
gacagcgtggccttcgaggacgtggccgtgaacttcacccaggaggagtgggccctgctgggccccagccagaagagcctgtacagaaacgtgatgcaggagaccatcagaaacctggactgcatcgagatgaagtgggaggaccagaacatcggcgaccagtgccagaacgccaagagaaacctgagaagccacacctgcgagatcaaggacgacagccagtgcggcgagaccttcggc

SEQ ID NO: 1141 - DNA, 240 bp, synthetic construct
  source 1..240: mol_type = other DNA
gccgccggctggctgaccacctggagccagaacagcgtgaccttccaggaggtggccgtggacttcagccaggaggagtgggccctgctggaccccgcccagaagaacctgtacaaggacgtgatgctggagaacttcagaaacctggccagcgtgggctaccagctgtgcagacacagcctgatcagcaaggtggaccaggagcagctgaagaccgacgagagaggcatcctgcagggc

SEQ ID NO: 1142 - DNA, 240 bp, synthetic construct
  source 1..240: mol_type = other DNA
gagaaccccagaaaccagctgatggccctgatgctgctgaccgcccagccccaggagctggtgatgttcgaggaggtgagcgtgtgcttcaccagcgaggagtgggcctgcctgggccccatccagagagccctgtactgggacgtgatgctggagaactacggcaacgtgaccagcctggagtgggagaccatgaccgagaacgaggaggtgaccagcaagcccagcagcagccagaga

SEQ ID NO: 1143 - DNA, 240 bp, synthetic construct
  source 1..240: mol_type = other DNA
ctggagacctacaacagcctggtgagcctgcaggagctggtgagcttcgaggaggtggccgtgcacttcacctgggaggagtggcaggacctggacgacgcccagagaaccctgtacagagatgtgatgctggagacctacagcagcctggtgagcctgggccactgcatcaccaagcccgagatgatcttcaagctggagcagggcgccgagccctggatcgtggaggagacccccaac

SEQ ID NO: 1144 - DNA, 240 bp, synthetic construct
  source 1..240: mol_type = other DNA
agacacttcagaagacccgagccctgcagagagcccctggccagccccatccaggacagcgtggccttcgaggacgtggccgtgaacttcacccaggaggagtgggccctgctggacagcagccagaagaacctgtacagagaggtgatgcaggagacctgcagaaacctggccagcgtgggcagccagtggaaggaccagaacatcgaggaccacttcgagaagcccggcaaggacatc

SEQ ID NO: 1145 - DNA, 240 bp, synthetic construct
  source 1..240: mol_type = other DNA
gccgccgccgagctgaccgcccccgcccagggcatcgtgaccttcgaagacgtggccgtgtacttcagctggaaggagtggggcctgctggacgaggcccagaagtgcctgtaccacgacgtgatgctggagaacctgaccctgaccaccagcctgggcggcagcggcgccggagatgaggaggctccttaccagcagagcaccagcccccagagagtgagccaggtgagaatccccaag

SEQ ID NO: 1146 - DNA, 240 bp, synthetic construct
  source 1..240: mol_type = other DNA
gccgccaccttccagctgcccggccaccaggagatgcccctgaccttccaggacgtggccgtgtacttcagccaggccgagggcagacagctgggcccccagcagagagccctgtacagagatgtgatgctggagaactacggcaacgtggccagcctgggcttccccgtgcccaagcccgagctgatcagccagctggagcagggcaaggagctgtgggtgctgaacctgctgggcgcc

SEQ ID NO: 1147 - DNA, 240 bp, synthetic construct
  source 1..240: mol_type = other DNA
agaagcgaggccggcgagccccccagcagcctgcaggtgaagcccgagacacctgccagcgccgctgtggccgtggctgctgctgctgctcccaccaccacagccgagaagccccccgccgaggcccaggacgaacctgccgagagcctgagcgagttcaagcccttcttcggcaacatcatcatcaccgacgtgaccgccaactgcctgaccgtgaccttcaaggagtacgtgaccgtg

SEQ ID NO: 1148 - DNA, 240 bp, synthetic construct
  source 1..240: mol_type = other DNA
cacagaaccaccagaatcaagatcaccgagctgaacccccacctgatgtgcgccctgtgcggcggctacttcatcgacgccaccaccatcgtggagtgcctgcacagcttctgcaagacctgcatcgtgagatacctggagaccaacaagtactgccccatgtgcgacgtgcaggtgcacaagaccagacccctgctgagcatcagaagcgacaagaccctgcaggacatcgtgtacaag

SEQ ID NO: 1149 - DNA, 240 bp, synthetic construct
  source 1..240: mol_type = other DNA
gccagccaggagttcgaggtggaggccatcgtggacaagagacaggacaagaacggcaacacccagtacctggtgagatggaagggctacgacaagcaggacgacacctgggagcccgagcagcacctgatgaactgcgagaagtgcgtgcacgacttcaacagaagacagaccgagaagcagaagaagctgacctggaccaccaccagcagaatcttcagcaacaacgccagaagaaga

SEQ ID NO: 1150 - DNA, 240 bp, synthetic construct
  source 1..240: mol_type = other DNA
gccagcggcgacctgtacgaggtggagagaatcgtggacaagagaaagaacaagaagggcaagtgggagtacctgatcagatggaagggctacggcagcaccgaggacacctgggagcccgagcaccacctgctgcactgcgaggagttcatcgacgagttcaacggcctgcacatgagcaaggacaagagaatcaagagcggcaagcagagcagcaccagcaagctgctgagagacagc

SEQ ID NO: 1151 - DNA, 240 bp, synthetic construct
  source 1..240: mol_type = other DNA
cccagaggcaagcacgccttccagaccggctggctgaacgacctggtgaccaaggagagcatgaccttcaaggacgtggccgtggacatcacccaggaggactgggagctgatgagacccgtgcagaaggagctgtacaagaccgtgaccctgcagaactactggaacatggtgagcctgggcctgaccgtgtacagacccaccgtgatccccatcctggaggagccctggatggtgatc

SEQ ID NO: 1152 - DNA, 240 bp, synthetic construct
  source 1..240: mol_type = other DNA
accctgatcagaaaggccgacctggagaaccacaacaaggacggcggcttctggaccgtgatcgacggcaaggtgtacgacatcaaggacttccagacccagagcctgaccggcaacagcatcctggcccagttcgccggcgaggaccccgtggtggccctggaagctgctctgcagttcgaggacaccagagagagcatgcacgccttctgcgtgggccagtacctggagcccgaccag

SEQ ID NO: 1153 - DNA, 240 bp, synthetic construct
  source 1..240: mol_type = other DNA
gagaagaccaagatcggcaccatggtggaggaccacagaagcaacagctaccaggacagcgtgaccttcgacgacgtggccgtggagttcacccccgaggagtgggccctgctggacaccacccagaagtacctgtacagagatgtgatgctggagaactacatgaacctggccagcgtggacttcttcttctgcctgaccagcgagtgggagatccagcccagaaccaagagaagcagc

SEQ ID NO: 1154 - DNA, 240 bp, synthetic construct
  source 1..240: mol_type = other DNA
gcccacgagctggtgatgttcagagatgtggccatcgacgtgagccaggaggagtgggagtgcctgaaccccgcccagagaaacctgtacaaggaggtgatgctggagaactacagcaacctggtgagcctgggcctgagcgtgagcaagcccgccgtgatcagcagcctggagcagggcaaggagccctggatggtggtgagagaggagaccggcagatggtgccccggcacctggaag

SEQ ID NO: 1155 - DNA, 240 bp, synthetic construct
  source 1..240: mol_type = other DNA
gccttcgaggacgtggccgtgtacttcagccaggaggagtggggcctgctggacaccgcccagagagccctgtacagaagagtgatgctggacaacttcgccctggtggccagcctgggcctgagcaccagcagacccagagtggtgatccagctggagagaggcgaggagccctgggtgcccagcggcaccgacaccaccctgagcagaaccacctacagaagaagaaaccccggcagc

SEQ ID NO: 1156 - DNA, 240 bp, synthetic construct
  source 1..240: mol_type = other DNA
gcccagctgagaagaggccacctgaccttcagagatgtggccatcgagttcagccaggaggagtggaagtgcctggaccccgtgcagaaggccctgtacagagatgtgatgctggagaactacagaaacctggtgagcctgggcatctgcctgcccgacctgagcatcatcagcatgatgaagcagagaaccgagccctggaccgtggagaacgagatgaaggtggccaagaaccccgac

SEQ ID NO: 1157 - DNA, 240 bp, synthetic construct
  source 1..240: mol_type = other DNA
agcgaccacagcctgggcatcagcagaagcaagacccccgtggacgaccccatgagcctgctgtacaacatgaacgactgctacagcaagctgaaggagctggtgcccagcatcccccagaacaagaaggtgagcaagatggagatcctgcagcacgtgatcgactacatcctggacctgcagatcgccctggacagccaccccaccatcgtgagcctgcaccaccagagacccggccag

SEQ ID NO: 1158 - DNA, 240 bp, synthetic construct
  source 1..240: mol_type = other DNA
aaggaccccaacgagccccagaagcccgtgagcgcctacgccctgttcttcagagacacccaggccgccatcaagggccagaaccccaacgccaccttcggcgaggtgagcaagatcgtggccagcatgtgggacggcctgggcgaggagcagaagcaggtgtacaagaagaagaccgaggccgccaagaaggagtacctgaagcagctggccgcctacagagccagcctggtgagcaag

SEQ ID NO: 1159 - DNA, 240 bp, synthetic construct
  source 1..240: mol_type = other DNA
caggaggagaagcaggaggacgccgccatctgccccgtgaccgtgctgcccgaggagcctgtgaccttccaggacgtggccgtggacttcagcagagaggagtggggcctgctgggccccacccagagaaccgagtacagagatgtgatgctggagaccttcggccacctggtgagcgtgggctgggagaccaccctggagaacaaggagctggcccccaacagcgacatccccgaggag

SEQ ID NO: 1160 - DNA, 240 bp, synthetic construct
  source 1..240: mol_type = other DNA
aagagcaccaaggactggaagatggcccccaagctgatctggcccgagagccagagcctgctgaccttcgaggacatggccgtgtacttcagcgaggaggagtggcagctgctgggccccctggagaagaccctgtacaacgacgtgatgcaggacatctacgagaccgccatcagcctgggcaagcagagaaccggcaagatcatgggcatcgagatggccagcagcttcagcaaggag

SEQ ID NO: 1161 - DNA, 240 bp, synthetic construct
  source 1..240: mol_type = other DNA
gacgccagcagactgagcggcagagaccccagcagctggaccgtggaggacgtgatgcagttcgtgagagaggccgacccccagctgggcccccacgccgacctgttcagaaagcacgagatcgacggcaaggccctgctgctgctgagaagcgacatgatgatgaagtacatgggcctgaagctgggccccgccctgaagctgagctaccacatcgacagactgaagcagggcaagttc

SEQ ID NO: 1162 - DNA, 240 bp, synthetic construct
  source 1..240: mol_type = other DNA
gcctactgcctgaccaactgctaccagtacagcgtgaccttcgaggacaccgccgtggacttcacccaggaggagtggatcctgctggaccccgtgcagagaaacctgtacagagatgtgatgctggagaactacgagaacgtggccaaggtgggcttccagctgttcaagcccagcgtgatcagctggctggaggaggaggagctgagaaccctgcagcagggcgtgctgcaggactgg

SEQ ID NO: 1163 - DNA, 240 bp, synthetic construct
  source 1..240: mol_type = other DNA
gccagaagacccagaagcagaaaccccagcgcctggaccgtggaggacgtggtgtggttcgtgaaggacgccgacccccaggccctgggcccccacgtggagctgttcagaaagcacgagatcgacggcaacgccctgctgctgctgaagagcgacatggtgatgaagtacctgggcctgaagctgggccccgccctgaagctgtgctaccacatcgacaagctgaagcaggccaagttc

SEQ ID NO: 1164 - DNA, 240 bp, synthetic construct
  source 1..240: mol_type = other DNA
gccgtgaccttcgaggacgtgaccatcatcttcacctgggaggagtggaagttcctggacagcagccagaagagactgtacagagaggtgatgtgggagaactacaccaacgtgatgagcgtggagaactggaacgagagctacaagagccaggaggagaagttcagatacctggagtacgagaacttcagctactggcagggctggtggaacgccggcgcccagatgtacgagaaccag

SEQ ID NO: 1165 - DNA, 240 bp, synthetic construct
  source 1..240: mol_type = other DNA
gagctgagcgccatcggcgagcaggtgttcgccgtggagagcatcagaaagaagagagtgagaaagggcaaggtggagtacctggtgaagtggaagggctggccccccaagtacagcacctgggagcccgaggagcacatcctggaccccagactggtgatggcctacgaggagaaggaggagagagacagagccagcggctacagaaagagaggccccaagcccaagagactgctgctg

SEQ ID NO: 1166 - DNA, 240 bp, synthetic construct
  source 1..240: mol_type = other DNA
ggcggcgccggcgccagactgcccgccctgctggacgagcagcaggttaacgtgctgctgtacgacatgaacggctgctacagcagactgaaggagctggtgcccaccctgccccagaacagaaaggtgagcaaggtggagatcctgcagcacgtgatcgactacatcagagacctgcagctggagctgaacagcgagagcgaggtgggcacccccggcggcagaggcctgcctgtgaga

SEQ ID NO: 1167 - DNA, 240 bp, synthetic construct
  source 1..240: mol_type = other DNA
gtggtgatggccgccagccccggcagcctgcacagcccccagcagcttgctgaggaagccaccagaaagagagagctgagactgatgaagaacagagaggccgccaaggagtgcagaagaagaaagaaggagtacgtgaagtgcctggagagcagagtggccgtgctggaggtgcagaacaagaagctgatcgaggagctggagaccctgaaggacatctgcagccccaagaccgactac

SEQ ID NO: 1168 - DNA, 240 bp, synthetic construct
  source 1..240: mol_type = other DNA
agcctgctgggcagacccaagagaaagagagtgatcacctacgcccagagacaggccgccaacatcagagagagaaagagaatgttcaacctgaacgaggccttcgaccagctgagaagaaaggtgcccaccttcgcctacgagaagagactgagcagaatcgagaccctgagactggccatcgtgtacatcagcttcatgaccgagctgctggagagctgcgagaagaaggagagcggc

SEQ ID NO: 1169 - DNA, 240 bp, synthetic construct
  source 1..240: mol_type = other DNA
ggcggcggccccggcggcagacccggcagagagcccagacaaagacacaccgccaacgccagagagagagacagaaccaacagcgtgaacaccgccttcaccgccctgagaaccctgatccccaccgagcccgccgacagaaagctgagcaagatcgagaccctgagactggccagcagctacatcagccacctgggcaacgtgctgctggccggcgaggcctgcggcgatggacagccc

SEQ ID NO: 1170 - DNA, 240 bp, synthetic construct
  source 1..240: mol_type = other DNA
agcggcttcggctacagcctgccccagcagcagcccgccgccgtggctagaagaaacgagagagagagaaacagagtgaagctggtgaacctgggcttcgccaccctgagagagcacgtgcccaacggcgccgccaacaagaagatgagcaaggtggagaccctgagaagcgccgtggagtacatcagagccctgcagcagctgctggacgagcacgacgccgtgagcgccgccttccag

SEQ ID NO: 1171 - DNA, 240 bp, synthetic construct
  source 1..240: mol_type = other DNA
gcccccctgccccccagagaccccaacggcgccggccctgaatggagagagcccggagccgtgagcttcgccgacgtggccgtgtacttctgcagagaggagtggggctgcctgagacccgcccagagagccctgtacagagatgtgatgagagagacctacggccacctgagcgccctgggcatcggcggcaacaagcccgccctgatcagctgggtggaggaggaggccgagctgtgg

SEQ ID NO: 1172 - DNA, 240 bp, synthetic construct
  source 1..240: mol_type = other DNA
aagcagggcttcagcaaggaccccagcacctggagcgtggacgaggtgatccagttcatgaagcacaccgacccccagatcagcggccccctggccgacctgttcagacagcacgagatcgacggcaaggccctgttcctgctgaagagcgacgtgatgatgaagtacatgggcctgaagctgggccccgccctgaagctgtgctactacatcgagaagctgaaggagggcaagtacagc

SEQ ID NO: 1173 - DNA, 240 bp, synthetic construct
  source 1..240: mol_type = other DNA
ggcgccttcggcgtgtacgagtaccccttcgagcccgccttcatccagaagagaaacgagagagagagacagagagtgaagtgcgtgaacgagggctacgccagactgagaggccacctgcccggcgccctggccgagaagagactgagcaaggtggagaccctgagagccgccatcagatacatcaagtacctgcaggagctgctgagcagcgcccccgacggcagcaccccccctgct

SEQ ID NO: 1174 - DNA, 240 bp, synthetic construct
  source 1..240: mol_type = other DNA
agcggcggcggcagcccccagagctacgaggagctgcagacccagagagtgatggccaacgtgagagagagacagagaacccagagcctgaacgaggccttcgccgccctgagaaagatcatccccaccctgcccagcgacaagctgagcaagatccagaccctgaagctggccgccagatacatcgacttcctgtaccaggtgctgcagagcgacgagctggacagcaagatggccagc

SEQ ID NO: 1175 - DNA, 240 bp, synthetic construct
  source 1..240: mol_type = other DNA
ttcgtgcagcacagatgcgaccccgccagagagaagcccctgaagtgccccgactgcgagaagagattcaagtacgccagcgacctgcagagacacagaagagtgcacaccggcgagaagccctacaagtgccccaactgcgacaaggccttcaagcagagagagcacctgaacaagcaccagggcgtgcacgccagagagcagcagttcaagtgcgtgtggtgcggcgagagattcctg

SEQ ID NO: 1176 - DNA, 240 bp, synthetic construct
  source 1..240: mol_type = other DNA
aacctgaccgaggactgcatggtgttcgaggacgtggccatctacttcagccaggaggagtggggcatcctgaacgacgcccagagacacctgcacagcaacgtgatgctggagaacttcgccctgctgagcagcgtgggctgctggcacggcgccaaggacgaggaggtgcccagcaagcagtgcgtgagcgtgagagtgctgcaggtgaccatccccaagcccgccctgagcaccctg

SEQ ID NO: 1177 - DNA, 240 bp, synthetic construct
  source 1..240: mol_type = other DNA
aacaagaagctggaggccgtgggcaccggcatcgagcccaaggccatgagccagggcctggtgaccttcggcgacgtggccgtggacttcagccaggaggagtgggagtggctgaaccccatccagagaaacctgtacagaaaggtgatgctggagaactacagaaacctggccagcctgggcctgtgcgtgagcaagcccgacgtgatcagcagcctggagcagggcaaggagccctgg

SEQ ID NO: 1178 - DNA, 240 bp, synthetic construct
  source 1..240: mol_type = other DNA
gccgaggagttcgtgaccctgaaggacgtgggcatggacttcaccctgggcgactgggagcagctgggcctggagcagggcgacaccttctgggacaccgccctggacaactgccaggacctgttcctgctggacccccccagacccaacctgaccagccaccccgacggcagcgaggacctggagcccctggctggaggaagccctgaggccaccagccccgacgtgaccgagaccaag

SEQ ID NO: 1179 - DNA, 240 bp, synthetic construct
  source 1..240: mol_type = other DNA
gacagcctgagcggcagcagcctgtacagcaccagcgccaacctgcccgtgatgggccacgagaagttccccagcgacctggacctggacatgttcaacggcagcctggagtgcgacatggagagcatcatcagaagcgagctgatggacgccgacggcctggacttcaacttcgacagcctgatcagcacccagaacgtggtgggcctgaacgtgggcaacttcaccggcgccaagcag

SEQ ID NO: 1180 - DNA, 240 bp, synthetic construct
  source 1..240: mol_type = other DNA
ggcggctacagcagcgtgagcagctgcaacggctacggcagaatgggcctgctgcaccaggagaagctgcccagcgacctggacggcatgttcatcgagagactggactgcgacatggagagcatcatcagaaacgacctgatggacggcgacaccctggacttcaacttcgacaacgtgctgcccaaccagagcttcccccacagcgtgaagaccaccacccacagctgggtgagcggc

SEQ ID NO: 1181 - DNA, 240 bp, synthetic construct
  source 1..240: mol_type = other DNA
ttctacatccccgtgcagatccccggctaccagtacgtgagccccgagggcaactgcatcgagcacgtgcagcccaccagcgccttcatccagcagcccttcatcgacgaggaccccgacaaggagaagaagatcaaggagctggagatgctgctgatgagcgccgagaacgaggtgagaagaaagagaatccccagccagcccggcagcttcagcagctggagcggcagcttcctgatg

SEQ ID NO: 1182 - DNA, 240 bp, synthetic construct
  source 1..240: mol_type = other DNA
gaggcccagaacgtgagcagccacgtgccctaccccgtggccctgcacgtgaacatcgtgaacgtgccccagcccgccgccgccgccatccagagacactacaacgacgaggaccccgagaaggagaagagaatcaaggagctggagctgctgctgatgagcaccgagaacgagctgaagggccagcaggtgctgcccacccagaaccacacctgcagctaccccggctggcacagcacc

SEQ ID NO: 1183 - DNA, 240 bp, synthetic construct
  source 1..240: mol_type = other DNA
cccttcggcagcagccccgacgacctgctgtgcccccaccccgctgctgaaagccccagcgacgagggcgctctgctggaccagctgtacctggccctgagaaacttcgacggcctggaggagatcgacagagccctgggcatccccgagctggtgagccagagccaggccgtggaccccgagcagttcagcagccaggacagcaacatcatgctggagcagaaggcccccgtgttcccc

SEQ ID NO: 1184 - DNA, 240 bp, synthetic construct
  source 1..240: mol_type = other DNA
agccagcccggcgccctgatccccggcgacccccaggctatgagccaacctaacagaggccccagccccttcagccccgtgcagctgcaccagctgagagcccagatcctggcctacaagatgctggccagaggccagcccctgcccgagaccctgcagctggccgtgcaaggcaagagaaccctgcccggcctgcagcagcagcagcagcagcagcagcagcagcagcagcagcagcag

SEQ ID NO: 1185 - DNA, 240 bp, synthetic construct
  source 1..240: mol_type = other DNA
cccagacccgagctgcccctgcccgagggctgggaggaggccagagacttcgacggcaaggtgtactacatcgaccacaccaacagaaccaccagctggatcgaccccagagacagatacaccaagcccctgaccttcgccgactgcatcagcgacgagctgcccctgggctgggaggaggcctacgacccccaggtgggagactacttcatcgaccacaacaccaagaccacccagatc

SEQ ID NO: 1186 - DNA, 240 bp, synthetic construct
  source 1..240: mol_type = other DNA
ctgagaaacagcctggacgacctggtgggcccccccagcaacctggagggccagagcgacgagagagccctgctggaccagctgcacaccctgctgagcaacaccgacgccaccggcctggaggagatcgacagagccctgggcatccccgagctggtgaaccagggccaggccctggagcccaagcaggacgccttccagggccaggaggctgccgtgatgatggaccagaaggccggc

SEQ ID NO: 1187 - DNA, 240 bp, synthetic construct
  source 1..240: mol_type = other DNA
ctggacgccctgcccggcccctacgccgctgctgctgctggacctcttggagctgcccctgacagattccccgccgacctggacctggacatgttcagcggcagcctggagtgcgacgtggagagcatcatcctgaacgacttcatggacagcgacgagatggacttcaacttcgacagcgccctgccccccccccctcctggacttgctggagctcctcctcccaaccagagctgggtg

SEQ ID NO: 1188 - DNA, 240 bp, synthetic construct
  source 1..240: mol_type = other DNA
gccagcatgccccccacccccgaggcccagggccccatcctgttcgaggaccttgctgtgtacttcagccaggaggagtgcgtgaccctgcaccccgcccagagaagcctgagcaaggacggcaccaaggagagcctggaggacgccgccctgatgggcgaggagggcaagcccgagatcaaccagcagctgagcctggagagcatggagctggacgagctggccctggagaagtacccc

SEQ ID NO: 1189 - DNA, 240 bp, synthetic construct
  source 1..240: mol_type = other DNA
ggcagccccagctacggcagccccgaggacaccgacagcttctggaaccccaacgccttcgagaccgacagcgacctgcccgccggctggatgagagtgcaggacaccagcggcacctactactggcacatccccaccggcaccacccagtgggagccccccggaagagctagccctagccagggcagcagcccccaggaggagagccagctgacctggaccggcttcgcccacggcgag

SEQ ID NO: 1190 - DNA, 240 bp, synthetic construct
  source 1..240: mol_type = other DNA
gagtgcagcgaggccggcctgctgcaggagggcgtgcagcccgaggagttcgtggctatcgccgactacgccgccaccgacgagacccagctgagcttcctgagaggcgagaagatcctgatcctgagacagaccaccgccgactggtggtggggcgagagagccggctgctgcggctacatccccgccaaccacgtgggcaagcacgtggacgagtacgaccccgaggacacctggcag

SEQ ID NO: 1191 - DNA, 240 bp, synthetic construct
  source 1..240: mol_type = other DNA
aactacagcttcctgcccctggtgcacaacatcatcaagtgcatggacaaggacagccccgaggtgcaccaggacctgaacgccctgaagagcaagttccaggagatgagaaagctgatcagcaccatgcccggcatccacctgagccccgagcagcagcagcagcagctgcagagcctgagagagcaggtgagaaccaagaacgagctgctgcagaagtacaagagcctgtgcatgttc

SEQ ID NO: 1192 - DNA, 240 bp, synthetic construct
  source 1..240: mol_type = other DNA
gccggcgaggacagcaagagcgagaacggcgagaacgcccccatctactgcatctgcagaaagcccgacatcaactgcttcatgatcggctgcgacaactgcaacgagtggttccacggcgactgcatcagaatcaccgagaagatggccaaggccatcagagagtggtactgcagagagtgcagagagaaggaccccaagctggagatcagatacagacacaagaagagcagagagaga

SEQ ID NO: 1193 - DNA, 240 bp, synthetic construct
  source 1..240: mol_type = other DNA
ggccccaacatcatcctgaccggcgacagcagccccggcttcagcaaggagatcgccgccgccctggctggcgtgcctggattcgaggtgagcgctgctggactggagctgggcctgggcctggaggacgagctgagaatggagcccctgggcctggagggcctgaacatgctgagcgacccctgcgccctgctgcccgaccccgctgtggaggagagcttcagaagcgacagactgcag

SEQ ID NO: 1194 - DNA, 240 bp, synthetic construct
  source 1..240: mol_type = other DNA
gaggcctgcaacagccacgcctgccagtgggacggcggcgactgcagcctgaccatggagaacccctgggccaactgcagcagccccctgccctgctgggactacatcaacaaccagtgcgacgagctgtgcaacaccgtggagtgcctgttcgacaacttcgagtgccagggcaacagcaagacctgcaagtacgacaagtactgcgccgaccacttcaaggacaaccactgcgaccag

SEQ ID NO: 1195 - DNA, 240 bp, synthetic construct
  source 1..240: mol_type = other DNA
agcggcggcgaggaggccaacctgctgctgcccgagctgggcagcgctttctacgacatggccagcggcttcaccgtgggcaccctgagcgagaccagcaccggcggacccgctacccccacatggaaggagtgccccatctgcaaggagagattccccgccgagagcgacaaggacgccctggaggaccacatggacggccacttcttcttcagcacccaggaccccttcaccttcgag

SEQ ID NO: 1196 - DNA, 240 bp, synthetic construct
  source 1..240: mol_type = other DNA
agacacggccacagcagcagcgaccccgtgtacccctgcggcatctgcaccaacgaggtgaacgacgaccaggacgccatcctgtgcgaggccagctgccagaagtggttccacagaatctgcaccggcatgaccgagaccgcctacggcctgctgaccgccgaggccagcgctgtgtggggatgcgacacctgcatggccgacaaggacgtgcagctgatgagaaccagagagaccttc

SEQ ID NO: 1197 - DNA, 240 bp, synthetic construct
  source 1..240: mol_type = other DNA
ctggtgggcagcagcctggagggcgccgtgaccccccagacaagcgcttggctgccccctaccagcgctgagcacgaccacagcctgagctgcgtggtgaccccccaggacggcgagaccagcgcccagatgatcgaggagaacctgaactgcctgggccacctgagcaccatcatccacgaggccaacgaggagcagggcaacagcatgatgaacctggactggagctggctgaccgag

SEQ ID NO: 1198 - DNA, 240 bp, synthetic construct
  source 1..240: mol_type = other DNA
gacctggccccccccagaaaggccctgttcacctaccccaagggcgccggcgagatgctggaggacggcagcgagagattcctgtgcgagagcgtgttcagctaccaggtggccagcaccctgaagcaggtgaagcacgaccagcaggtggccagaatggagaagctggccggcctggtggaggagctggaggccgacgagtggagattcaagcccatcgagcagctgctgggcttcacc

SEQ ID NO: 1199 - DNA, 240 bp, synthetic construct
  source 1..240: mol_type = other DNA
gccatgcagcacttcagccagagattcctgtaccagagcagcagcgccagcaccgaccacgaccccctgggcccccttcctcctggatgggagaagagacaggacaacggcagagtgtactacgtgaaccacaacaccagaaccacccagtgggaggaccccagaacccagggcatgatccaggagcccgccctgccccccggatgggagatgaagtacaccagcgagggcgtgagatac

SEQ ID NO: 1200 - DNA, 240 bp, synthetic construct
  source 1..240: mol_type = other DNA
aacagccagaacagcttcctggtgcaggaggtgatggaggaggagtggaacgccctgcagagcgtggagaactgccccgaggacctggcccagctggaggagctgatcgacatggccgtgctggaggagatccagcaggagctgatcaaccaggagcagagcatcatcagcgagtacgagaagagcctgcagttcgacgagaagtgcctgagcatcatgctggccgagtgggaggccaac

SEQ ID NO: 1201 - DNA, 240 bp, synthetic construct
  source 1..240: mol_type = other DNA
aagagcgacaagcagatcaagaacggcgagtgcgacaaggcctacctggacgagctggtggagctgcacagaagactgatgaccctgagagagagacacatcctgcagcagatcgtgaacctgatcgaggagaccggccacttccacatcaccaacaccaccttcgacttcgacctgtgcagcctggacaagaccaccgtgagaaagctgcagagctacctggagaccagcggcaccagc

SEQ ID NO: 1202 - DNA, 240 bp, synthetic construct
  source 1..240: mol_type = other DNA
ctggagtacagcctgctgggcgagcactgggactacgacgccctgttcgagacccagcccggcctggtgaccatcaagaacctggccgtggacttcagacagcagctgcaccccgcccagaagaacttctgcaagaacggcatctgggagaacaacagcgacctgggcagcgccggccactgcgtggccaagcccgacctggttagcctgctggagcaggagaaggagccctggatggtg

SEQ ID NO: 1203 - DNA, 240 bp, synthetic construct
  source 1..240: mol_type = other DNA
gccatgcagcagttcaaccagagatacctgtacagcgccagcatgctggccgccgagaacgacccctacggccccctgcctcctggctgggagaagagagtggacagcaccgacagagtgtacttcgtgaaccacaacaccaagaccacccagtgggaggaccccagaacccagggcctgcagaacgaggagcccctgcccgagggctgggagatcagatacaccagagagggcgtgaga

SEQ ID NO: 1204 - DNA, 240 bp, synthetic construct
  source 1..240: mol_type = other DNA
gagtacggcctgaccgacaacgtggagagaatcgtggagaacgagaagatcaacgccgagaagagcagcaagcagaaggtggacctgcagagcctgcccaccagagcctacctggaccagaccgtggtgcccatcctgctgcagggcctggccgtgctggccaaggagagaccccccaaccccatcgagttcctggccagctacctgctgaagaacaaggcccagttcgaggacagaaac

SEQ ID NO: 1205 - DNA, 240 bp, synthetic construct
  source 1..240: mol_type = other DNA
cacatgaactgggacgacctgctggcctggagagtggacccccccttcagaccctgcctgcagagcgaggaggacgtgagccagttcgacaccagattcaccagacagacccccgtggacagccccgacgacaccgccctgagcgagagcgccaaccaggccttcctgggcttcacctacgtggcccccagcgtgctggacagcatcaaggagggcttcagcttccagcccaagctgaga

SEQ ID NO: 1206 - DNA, 240 bp, synthetic construct
  source 1..240: mol_type = other DNA
agcggcccccagcccccccccggcctggtttacccttgcggagcttgcagaagcgaggtgaacgacgaccaggacgccatcctgtgcgaggccagctgccagaagtggttccacagagagtgcaccggcatgaccgagagcgcctacggcctgctgaccaccgaggccagcgccgtgtgggcttgcgacctgtgcctgaagaccaaggagatccagagcgtgtacatcagagagggcatg

SEQ ID NO: 1207 - DNA, 240 bp, synthetic construct
  source 1..240: mol_type = other DNA
gtggaggtgcaggagcactacgacagcttcttcgaggaggtgttcaccgagctgcaggagaagtacggcgagatcgaggagatgaacgtgtgcgacaacctgggcgaccacctggtgggcaacgtgtacgtgaagttcagaagagaggaggacggcgagagagccgtggccgagctgagcaacagatggttcaacggccaggccgtgcacggcgagctgagccccgtgaccgacttcaga

SEQ ID NO: 1208 - DNA, 240 bp, synthetic construct
  source 1..240: mol_type = other DNA
agcggcctgatcatccccctgaccatcagcggcggcagcggccccagacccctgaaccccgtgacccaagctcctctgccccccggatgggagcagagagtggaccagcacggcagagtgtactacgtggaccacgtggagaagagaaccacctgggacagacccgagcccctgccccccggctgggagagaagagtggacaacatgggcagaatctactacgtggaccacttcaccaga

SEQ ID NO: 1209 - DNA, 240 bp, synthetic construct
  source 1..240: mol_type = other DNA
agcaagcccgagaagatcctgaagaagggcacctacgacaaggcctacaccgacgagctggtggagctgcacagaagactgatggccctgagagagagaaacgtgctgcagcagatcgtgaacctgatcgaggagaccggccacttcaacgtgaccaacaccaccttcgacttcgacctgttcagcctggacgagaccaccgtgagaaagctgcagagctgcctggaggccgtggccacc

SEQ ID NO: 1210 - DNA, 240 bp, synthetic construct
  source 1..240: mol_type = other DNA
agccagaccgtgcccgagcccgaccagggccccgtgagccagcctgttcctgagcctgacctgccctgcgacctgagacacctgaacaccgagcccatggagatcttcagaaactgcgtgaagatcgaggagatcatgcccaacggcgaccccctgctggccggccagaacaccgtggacgaggtgtacgtgagcagacccagccacttctacaccgacggccccctgatgcccagcgac

SEQ ID NO: 1211 - DNA, 240 bp, synthetic construct
  source 1..240: mol_type = other DNA
ctgtgccacatcctggactacagcttcggcggcggcgccggcagagacatccccccccctctgatcgaggaggcttgcgagctgcccgagtgccaggaggacgccggcaacaaggtgtgcagcctgcagtgcaacaaccacgcctgcggctgggacggcggcgactgcagcctgaacttcaacgacccctggaagaactgcacccagagcctgcagtgctggaagtacttcagcgacggc

SEQ ID NO: 1212 - DNA, 240 bp, synthetic construct
  source 1..240: mol_type = other DNA
aactgcggcagcctgcccaacaccatcctgcccgaggacagcagcaccagcctgttcaaggacctgaacagcgccctggccggcctgcccgaggtgagcctgaacgtggacacccccttccccctggaggaggagctgcagatcgagcccctgagcctggacggcctgaacatgctgagcgacagcagcatgggcctgctggaccccagcgtggaggagaccttcagagccgacagactg

SEQ ID NO: 1213 - DNA, 240 bp, synthetic construct
  source 1..240: mol_type = other DNA
cacgccagcggcatcggcagagtggccgccaccagcctgggcaacctgaccaaccacggcagcgaggacctgcccctgccccccggatggagcgtggactggaccatgagaggcagaaagtactacatcgaccacaacaccaacaccacccactggagccaccccctggagagagagggcctgccccccggatgggagagagtggagagcagcgagttcggcacctactacgtggaccac

SEQ ID NO: 1214 - DNA, 240 bp, synthetic construct
  source 1..240: mol_type = other DNA
cccctgagcctgggcgaggacttctacagagaggccatcgagcactgcagaagctacaacgccagactgtgcgccgagagaagcctgagactgcccttcctggacagccagaccggcgtggcccagaacaactgctacatctggatggagaagacccacagaggccccggcctggcccccggccagatctacacctaccccgctagatgctggagaaagaagagaagactgaacatcctg

SEQ ID NO: 1215 - DNA, 240 bp, synthetic construct
  source 1..240: mol_type = other DNA
aaggagaacctgctggccggccccagcgagaacgaccccaacctgttcgtggccctgtacgacttcgtggccagcggcgacaacaccctgagcatcaccaagggcgagaagctgagagtgctgggctacaaccacaacggcgagtggtgcgaggcccagaccaagaacggccagggctgggtgcccagcaactacatcacccccgtgaacagcctggagaagcacagctggtaccacggc

SEQ ID NO: 1216 - DNA, 240 bp, synthetic construct
  source 1..240: mol_type = other DNA
tactacgacctgatcagcggcgccagccagtgggagaagcccgagggcttccagggcgacctgaagaagaccgccgtgaagaccgtgtgggtggagggcctgagcgaggacggcttcacctactactacaacaccgagaccggcgagagcagatgggagaagcccgacgacttcatcccccacaccagcgacctgcccagcagcaaggtgaacgagaacagcctgggcaccctggacgag

SEQ ID NO: 1217 - DNA, 240 bp, synthetic construct
  source 1..240: mol_type = other DNA
cccgagcccgccgccgcccccgtgagcaccagcgagctgaagaaggtggtggctctgtacgactacatgcccatgaacgccaacgacctgcagctgagaaagggcgacgagtacttcatcctggaggagagcaacctgccctggtggagagccagagacaagaacggccaggagggctacatccccagcaactacgtgaccgaggccgaggacagcatcgagatgtacgagtggtacagc

SEQ ID NO: 1218 - DNA, 240 bp, synthetic construct
  source 1..240: mol_type = other DNA
atgtacaccaagaagaacgtgcccagcaagagcaaggccgccgccagcgccaccagagagtggaccgagcaggagaccctgctgctgctggaggccctggagatgtacaaggacgactggaacaaggtgagcgagcacgtgggcagcagaacccaggacgagtgcatcctgcacttcctgagactgcccatcgaggacccctacctggaggacagcgaggccagcctgggccccctggcc

SEQ ID NO: 1219 - DNA, 240 bp, synthetic construct
  source 1..240: mol_type = other DNA
ggcgccgtgaacggcgccgtgggcaccaccttccagccccagaaccccctgctgggcagagcctgcgagagctgctacgccacccagagccaccagtggtacagctggggcccccccaacatgcagtgcagactgtgcgccatctgctggctgtactggaagaagtacggcggcctgaagatgcccacccagagcgaggaggagaagctgagccccagccccaccaccgaggaccccaga

SEQ ID NO: 1220 - DNA, 240 bp, synthetic construct
  source 1..240: mol_type = other DNA
ggcgccgccggcagccccgcccagcagcacgcccacctgagacaacaaagctacgacgtgaccgacgagctgcccctgccccccggctgggagatgaccttcaccgccaccggacagagatacttcctgaaccacatcgagaagatcaccacctggcaggaccccagaaaggccatgaaccagcccctgaaccacatgaacctgcaccccgccgtgagcagcacccccgtgccccagaga

SEQ ID NO: 1221 - DNA, 240 bp, synthetic construct
  source 1..240: mol_type = other DNA
accgtgacctacctgggcaagttcgccttcgacagccccagcaactggtgccaggacaacatcatcagcctgatgagcgccggcatcctgggcgtgccccccgctagcggagctctgagcacacagaccagcaccgccagcatggtgcagcccccccagggcgacgtggaggccatgtaccccgcccttcctccctacagcaactgcggcgacctgtacagcgagcccgtgagcttccac

SEQ ID NO: 1222 - DNA, 240 bp, synthetic construct
  source 1..240: mol_type = other DNA
tacagcccctactgcctgacccaggacgagttccaccccttcatcgaggccctgctgccccacgtgagagccttcagctacacctggttcaacctgcaggccagaaagagaaagtacttcaagaagcacgagaagagaatgagcaaggacgaggagagagccgtgaaggacgagctgctgggcgagaagcccgagatcaagcagaagtgggccagcagactgctggccaagctgagaaag

SEQ ID NO: 1223 - DNA, 240 bp, synthetic construct
  source 1..240: mol_type = other DNA
cagggccaccccttcttcagaaacgtggactgggacatgatggagcagaagcaggtggtgccccccttcaagcccaacatcagcggcgagttcggcctggacaacttcgacagccagttcaccaacgagcccgtgcagctgacccccgacgacgacgacatcgtgagaaagatcgaccagagcgagttcgagggcttcgagtacatcaaccccctgctgatgagcgccgaggagtgcgtg

SEQ ID NO: 1224 - DNA, 240 bp, synthetic construct
  source 1..240: mol_type = other DNA
agcttcctggtgaacatgaagctggaggccgtggacagaagaaaccccgccctgatcagagtggccagcgtggaggacgtggaggaccacagaatcaagatccacttcgacggctggagccacggctacgacttctggatcgacgccgaccaccccgacatccaccccgccggctggtgcagcaagaccggacaccccctgcagcctcctctgggccccagagagcccagcagcgctagc

SEQ ID NO: 1225 - DNA, 240 bp, synthetic construct
  source 1..240: mol_type = other DNA
ctgcagtgcaacaaccacgcctgcggctgggacggcggcgactgcagcctgaacttcaacgacccctggaagaactgcacccagagcctgcagtgctggaagtacttcagcgacggccactgcgacagccagtgcaacagcgccggctgcctgttcgacggcttcgactgccagagagccgagggccagtgcaaccccctgtacgaccagtactgcaaggaccacttcagcgacggccac

SEQ ID NO: 1226 - DNA, 240 bp, synthetic construct
  source 1..240: mol_type = other DNA
ctgctggtgcagagaaccgagggcttcagcggcctggacgtggcccacctgtgccaggaggccgtggtgggacctctgcacgccatgcccgccaccgacctgagcgccatcatgcccagccagctgagacccgtgacctaccaggacttcgagaacgccttctgcaagatccagcccagcatcagccagaaggagctggacatgtacgtggagtggaacaagatgttcggctgcagccag

SEQ ID NO: 1227 - DNA, 240 bp, synthetic construct
  source 1..240: mol_type = other DNA
agactgggcgagcaggaggccaagagaaacggcaccggcatcaacccctactgcgccctgatcgaggaggcctacggcctggacaagatcgagttcctgcagagccacgagaaccaggagatctaccagaaggccttcgacctgatcgagcactacttcggcaccgaggacgaggacagcagcatcgccccccaggtggacctgaaccagcagcagtacatcttccagcagtgcgaggcc

SEQ ID NO: 1228 - DNA, 240 bp, synthetic construct
  source 1..240: mol_type = other DNA
caggaggagaacgtgagagacacccagcaggtgaccaccctgcagctgccccccagcagagtgagccccttcaaggacatgatcctgtgcttcagcgaggaggactggagcctgctggaccccgcccagaccggcttctacggcgagttcatcatcggcgaggactacggcgtgagcatgccccccaacgacctggccgcccagcccgaccttagccagggcgaggagaacgagcccaga

SEQ ID NO: 1229 - DNA, 213 bp, synthetic construct
  source 1..213: mol_type = other DNA
gatgccaagtcccttgaggaggagtctcggaccctggttacctttaaggatgtgttcgtcgacttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1230 - DNA, 213 bp, synthetic construct
  source 1..213: mol_type = other DNA
gatgccaagtccttgaccgctgaggaggagaccctggtcactttcaaggacgtttttgtggatttcacgagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1231 - DNA, 213 bp, synthetic construct
  source 1..213: mol_type = other DNA
gatgctaagtccctgaccgccgatgatgacacccttgtgacttttaaggatgtcttcgttgacttcacgagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1232 - DNA, 213 bp, synthetic construct
  source 1..213: mol_type = other DNA
gatgctaagtctctgaccgcctggtcccggactcttgttacgtttaaggacgtgttcgtcgatttcaccagggaagaatggaaactactagacacagcacaacaaatagtatacagaaacgtaatgctagagaactacaaaaacctagtaagcctaggataccaactcacaaaaccagacgtaatactaagactagaaaaaataataatacca

SEQ ID NO: 1233 - DNA, 213 bp, synthetic construct
  source 1..213: mol_type = other DNA
gatgccaagtcccttaccccccctccccggactctggtgacgtttaaggatgtcttcgttgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1234 - DNA, 213 bp, synthetic construct
  source 1..213: mol_type = other DNA
gataataagtcccttaccgcctggtctcggacgctggttactttcaaggacgtcttcgtggattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1235 - DNA, 213 bp, synthetic construct
  source 1..213: mol_type = other DNA
gatgctaagtctttgactgcctggtcccctccccccgttaccttcaaggatgtgtttgtcgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1236 - DNA, 213 bp, synthetic construct
  source 1..213: mol_type = other DNA
gacgccaagtccctgacggaggagtctcggacccttgtgacttttaaggatgtcttcgttgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1237 - DNA, 213 bp, synthetic construct
  source 1..213: mol_type = other DNA
gacgctaagtcccttaccgccgccgcgaggaccctggttacttttaaggatgtcttcgtggatttcacgcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1238 - DNA, 213 bp, synthetic construct
  source 1..213: mol_type = other DNA
gatgccaagtctgtgaccgcttggtcccggacgcttgtcaccttcaaggacgtttttgtggatttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1239 - DNA, 213 bp, synthetic construct
  source 1..213: mol_type = other DNA
gatgctaagtctcttaccgccccctcccggactctggtcaccttcaaggacgtgttcgttgattttacgagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1240 - DNA, 213 bp, synthetic construct
  source 1..213: mol_type = other DNA
gacgccaagtcccttaccgctggcgggcggaccctggtgactttcaaggatgttttcgtcgattttacgagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1241 - DNA, 213 bp, synthetic construct
  source 1..213: mol_type = other DNA
gatgccaagtctcttaataataattcccggactctggtgacctttaaggacgttttcgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1242 - DNA, 213 bp, synthetic construct
  source 1..213: mol_type = other DNA
gatgccaagtcccttaccgctcagcagcagactctggtcacgttcaaggacgtttttgtggatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1243 - DNA, 213 bp, synthetic construct
  source 1..213: mol_type = other DNA
gatgctaagtctcttactgcctggtcccggcctcccgtgaccttcaaggatgtcttcgttgactttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1244 - DNA, 213 bp, synthetic construct
  source 1..213: mol_type = other DNA
gacgctaagtccctgaccgccacgacccggactcttgtcactttcaaggatgtgttcgttgattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1245 - DNA, 213 bp, synthetic construct
  source 1..213: mol_type = other DNA
gatgccaagtctcttaccgcttggtccagggaggaggtcactttcaaggacgtgttcgttgattttacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1246 - DNA, 213 bp, synthetic construct
  source 1..213: mol_type = other DNA
gatgccaagtcccttaccgctgatgaccggaccctggtcacgttcaaggatgtgtttgttgacttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1247 - DNA, 213 bp, synthetic construct
  source 1..213: mol_type = other DNA
gatgccaagtcccttacgcagcagcagcggactctggttaccttcaaggacgtgtttgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1248 - DNA, 213 bp, synthetic construct
  source 1..213: mol_type = other DNA
gacgccaagtccgaggaggagtggtctaggacccttgtcacctttaaggatgttttcgtggatttcactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1249 - DNA, 213 bp, synthetic construct
  source 1..213: mol_type = other DNA
gatgccaagtcccttaccgacgactctaggacgctggttacttttaaggatgtgttcgtcgatttcacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1250 - DNA, 213 bp, synthetic construct
  source 1..213: mol_type = other DNA
gacgctaagtccctgacggccaataatcggacccttgttacctttaaggatgtgttcgtcgatttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1251 - DNA, 213 bp, synthetic construct
  source 1..213: mol_type = other DNA
aataataattctcttaccgcctggtccaggacgctggtgaccttcaaggatgtttttgtcgatttcactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1252 - DNA, 213 bp, synthetic construct
  source 1..213: mol_type = other DNA
gatgccaagtccctgggcggtgggtctcggacccttgttacttttaaggacgtgttcgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1253 - DNA, 213 bp, synthetic construct
  source 1..213: mol_type = other DNA
gatgccaagtcccttaccgacgacgataggactctggttaccttcaaggatgtgttcgtcgattttacgcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1254 - DNA, 213 bp, synthetic construct
  source 1..213: mol_type = other DNA
gatgccaagtctctggacgatgattcccggacccttgtcacctttaaggatgtgttcgttgacttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1255 - DNA, 213 bp, synthetic construct
  source 1..213: mol_type = other DNA
gacgccaagtctctgactgcttggtcccggaccctggtgacgtttaaggatgtcttcgttgatttcaccagggaagaatggaaactactagacacagcacaacaaatagtatacagaaacgtaatgctagaaaactacaaaaacctagtaagcctaggataccaactaacaaaaccagacgtaataaaaaaactagaaaaaggagaagaacca

SEQ ID NO: 1256 - DNA, 213 bp, synthetic construct
  source 1..213: mol_type = other DNA
gatgctaagtcccttaccgccgattctaggacgctggtgaccttcaaggatgtctttgttgacttcactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1257 - DNA, 213 bp, synthetic construct
  source 1..213: mol_type = other DNA
gatgccaagtctctgaagaagaagtcccggacccttgtcactttcaaggacgtttttgtggatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1258 - DNA, 213 bp, synthetic construct
  source 1..213: mol_type = other DNA
gatgccaagtctctgaccgctgagtccaggactcttgttacgttcaaggatgtctttgtggacttcacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1259 - DNA, 213 bp, synthetic construct
  source 1..213: mol_type = other DNA
gacgccaagtcccttacggctccccctcggaccctggtcactttcaaggatgttttcgtggattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1260 - DNA, 213 bp, synthetic construct
  source 1..213: mol_type = other DNA
gatgccaagtcccttactgctcctccccccacgctggttacctttaaggacgtgttcgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1261 - DNA, 213 bp, synthetic construct
  source 1..213: mol_type = other DNA
gatgctaagtccctgacggcctgggacgatactttggtgacctttaaggacgtcttcgttgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1262 - DNA, 213 bp, synthetic construct
  source 1..213: mol_type = other DNA
gacgctaagtcccttacggccaataataatactctggttacctttaaggatgtcttcgtggatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1263 - DNA, 213 bp, synthetic construct
  source 1..213: mol_type = other DNA
gatgccaagtctctgactgctatgtccaggacccttgtcaccttcaaggacgtgtttgttgatttcacgcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1264 - DNA, 213 bp, synthetic construct
  source 1..213: mol_type = other DNA
gatgccaagtcccttaccgcttggaataataatctggtgactttcaaggatgtttttgtcgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1265 - DNA, 213 bp, synthetic construct
  source 1..213: mol_type = other DNA
gatgctaagtccctgaccgcctggtctaataatttggttaccttcaaggatgtgtttgtcgacttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1266 - DNA, 213 bp, synthetic construct
  source 1..213: mol_type = other DNA
gacgccaagtcccttacgcccccttctcggactctggtgacctttaaggatgtcttcgttgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1267 - DNA, 213 bp, synthetic construct
  source 1..213: mol_type = other DNA
gacgccaagtccctgacggaggaggagcggactcttgtgacctttaaggatgttttcgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1268 - DNA, 213 bp, synthetic construct
  source 1..213: mol_type = other DNA
gatgctaagtctttggccgcggcctccaggacccttgtgactttcaaggacgtttttgtcgatttcacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1269 - DNA, 213 bp, synthetic construct
  source 1..213: mol_type = other DNA
gatgctaagtccctgaccgcccagcagcggacgcttgttactttcaaggatgtctttgtggacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1270 - DNA, 213 bp, synthetic construct
  source 1..213: mol_type = other DNA
gatgccaagtcccttaccgcttggtctgaggaggaggttactttcaaggatgtgtttgtcgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1271 - DNA, 213 bp, synthetic construct
  source 1..213: mol_type = other DNA
gatgccaagtctctgactcagcagtcccggacccttgtgacgttcaaggacgttttcgtcgattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1272 - DNA, 213 bp, synthetic construct
  source 1..213: mol_type = other DNA
gacgctaagtcccttacggcctggtctaggaccctggtcacctttaaggatgttttcgtggatttcactcgggaagaatggaaactactagacacagcacaacaaatagtatacagagaagtaatgctagaaaactacaaaaacctagtaagcctaggataccaactaacaaaaccagacgtaatactaagactagaaaaaggagaagaacca

SEQ ID NO: 1273 - DNA, 213 bp, synthetic construct
  source 1..213: mol_type = other DNA
gatgccaagtctcttaccgcttggtccaggcccctggtgactttcaaggatgtctttgttgacttcacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1274 - DNA, 213 bp, synthetic construct
  source 1..213: mol_type = other DNA
gacgctaagtccaccaccgcctggtctaggactcttgttacgttcaaggatgtgtttgtcgatttcactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1275 - DNA, 213 bp, synthetic construct
  source 1..213: mol_type = other DNA
gacgccaagtctcttaccgcttggtccaataataatgttactttcaaggatgtgtttgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1276 - DNA, 213 bp, synthetic construct
  source 1..213: mol_type = other DNA
gatgctaagtcccttactgcctgggacgatgatctggtcaccttcaaggacgtttttgtggatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1277 - DNA, 213 bp, synthetic construct
  source 1..213: mol_type = other DNA
gatgctaagtcccttactgccgcctctcggacgctggtgaccttcaaggacgttttcgtcgattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1278 - DNA, 213 bp, synthetic construct
  source 1..213: mol_type = other DNA
gacgccaagtctttgacggctggctcccggacccttgtgactttcaaggatgtcttcgttgattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1279 - DNA, 213 bp, synthetic construct
  source 1..213: mol_type = other DNA
gacgctaagagtctgactgcctcttcccggacccttgtcacgttcaaggatgttttcgtggattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1280 - DNA, 213 bp, synthetic construct
  source 1..213: mol_type = other DNA
gatgccaagtccctgacgtctagttcaaggactcttgtgacctttaaggacgtcttcgttgatttcacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1281 - DNA, 213 bp, synthetic construct
  source 1..213: mol_type = other DNA
gatgctaaggatgacactgcctggtcccggacgcttgtgacctttaaggatgtcttcgttgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1282 - DNA, 213 bp, synthetic construct
  source 1..213: mol_type = other DNA
gatgccaagtctctgaccgcttggtccaggacgcttgtgactttcaaggatgtcttcgttgactttacccgggaagaatggaaactactagacacagcacaacaaatagtatacagaaacgtaatgctagaaaactacaaaaacctagtaagcctaggataccaacacacaaaaccagacgtaatactaagactagaaaaaggagaagaacca

SEQ ID NO: 1283 - DNA, 213 bp, synthetic construct
  source 1..213: mol_type = other DNA
gatgccaagtcccttaccaagaagtctaggactctggttaccttcaaggacgtctttgtggatttcacgcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1284 - DNA, 213 bp, synthetic construct
  source 1..213: mol_type = other DNA
gatgccaagtcccttactgcttggtctgaggagctggtgaccttcaaggatgtctttgttgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1285 - DNA, 213 bp, synthetic construct
  source 1..213: mol_type = other DNA
gatgccaagtcccttaccgcttggcagcagcagctggtcactttcaaggacgtgttcgttgattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1286 - DNA, 213 bp, synthetic construct
  source 1..213: mol_type = other DNA
gatgccaagtcccttacggcttggtctaggaccaccgtgactttcaaggacgttttcgtcgattttactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1287 - DNA, 213 bp, synthetic construct
  source 1..213: mol_type = other DNA
gatgccaagtctcttaccgctaattccaggacgctggtcaccttcaaggatgtttttgtggacttcactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1288 - DNA, 213 bp, synthetic construct
  source 1..213: mol_type = other DNA
gacgccaagtctctgcagcagcagtcccggactcttgttaccttcaaggatgtgtttgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1289 - DNA, 213 bp, synthetic construct
  source 1..213: mol_type = other DNA
gatgctaagtcccttaccgcctggcctcccaccctggttacttttaaggatgtgttcgtcgacttcacgagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1290 - DNA, 213 bp, synthetic construct
  source 1..213: mol_type = other DNA
gacgctaaggaggaggaggcctggtccaggacccttgttacctttaaggatgtgttcgtcgatttcactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1291 - DNA, 213 bp, synthetic construct
  source 1..213: mol_type = other DNA
gatgccaagtccctgaccaataattctcggacccttgtcacttttaaggacgtgttcgttgatttcacgagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1292 - DNA, 213 bp, synthetic construct
  source 1..213: mol_type = other DNA
gatgctaagtctcttacggcctggtccaggaccgatgtgactttcaaggacgttttcgtcgattttacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1293 - DNA, 213 bp, synthetic construct
  source 1..213: mol_type = other DNA
gacgccaagtcccttactgctactaccaccacgctggttacctttaaggatgtcttcgtggatttcacaagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1294 - DNA, 213 bp, synthetic construct
  source 1..213: mol_type = other DNA
gatgccaagtcccttaccgcttggtctgatgacctggttaccttcaaggacgtcttcgtggattttactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1295 - DNA, 213 bp, synthetic construct
  source 1..213: mol_type = other DNA
gatgctaagtccctcaccgccaagtctaggaccttggtcacgttcaaggacgttttcgtggattttactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1296 - DNA, 213 bp, synthetic construct
  source 1..213: mol_type = other DNA
gatgccaagtctctgaccgcttggtcccggacccttgtcacgttcgaggatgttttcgtggactttactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1297 - DNA, 213 bp, synthetic construct
  source 1..213: mol_type = other DNA
gatgccaagtccctgactgcttggtctaggacccttgtgacctttgatgatgttttcgtcgacttcacgcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1298 - DNA, 213 bp, synthetic construct
  source 1..213: mol_type = other DNA
gacgctaagtcccttaccgcctggtctcggacgctggtgacctttgatgatgtcttcgttgatttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1299 - DNA, 213 bp, synthetic construct
  source 1..213: mol_type = other DNA
gacgctaagtcccttaccgcctggtctcagcagcaggttactttcaaggatgtctttgtggatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1300 - DNA, 213 bp, synthetic construct
  source 1..213: mol_type = other DNA
gatgctaagagccttaccgcctggtcccggaccctggtgactttcaaggacgtcttcgttgattttacgagggaagaatggaaactactagacacagcacaacaaatagtatacagaaacgtaatgctagaaaactacaaaaacctagtagaactaggataccaactaacaaaaccagacgtaatactaagactagaaaaaggagaagaacca

SEQ ID NO: 1301 - DNA, 213 bp, synthetic construct
  source 1..213: mol_type = other DNA
gacgccaagtcccttaccgcttgggaggaggagctggtcactttcaaggatgtttttgtggatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1302 - DNA, 213 bp, synthetic construct
  source 1..213: mol_type = other DNA
gatgctaagtccctgaccgcctggtctcggacccttgtgacgttcaaggacgtctttgttgatttcactagggaagaatggaaactactagacacagcacaacaagaagtatacagaaacgtaatgctagaaaactacaaaaacctagtaagcctaggataccaactaacaaaaccagacgtaatactaagactagaaaaaggagaagaacca

SEQ ID NO: 1303 - DNA, 213 bp, synthetic construct
  source 1..213: mol_type = other DNA
gatgccaagtctcttacggcttggtcccggacccccgttacctttaaggacgtcttcgtggatttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1304 - DNA, 213 bp, synthetic construct
  source 1..213: mol_type = other DNA
gacgccaagtcccttacggcttggtctcggactctggtcacctttaaggatgtgttcgttgatttcaccagggaagaatggaaactactagacacagcacaacaaatagtatacagaaacgtaatgctagaaaactacaaaaacctagtaagcctaggatacgaagaagaaaaaccagacgtaatactaagactagaaaaaggagaagaacca

SEQ ID NO: 1305 - DNA, 213 bp, synthetic construct
  source 1..213: mol_type = other DNA
gatgctaagtccctgactgcctggtctaggacccttgtcacctttaaggatgtgttcgttgatttcacgcgggaagaatggaaactactagacacagcacaacaagacgactacagaaacgtaatgctagaaaactacaaaaacctagtaagcctaggataccaactaacaaaaccagacgtaatactaagactagaaaaaggagaagaacca

SEQ ID NO: 1306 - DNA, 213 bp, synthetic construct
  source 1..213: mol_type = other DNA
gacgcgaagtccctgacggccgctgcccggactcttgttaccttcaaggatgtgttcgtcgattttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1307 - DNA, 213 bp, synthetic construct
  source 1..213: mol_type = other DNA
gatgctaagtcccttacggcccagtctcggactctggttaccttcaaggatgtcttcgtggactttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1308 - DNA, 213 bp, synthetic construct
  source 1..213: mol_type = other DNA
gatgctaagtccctcacggcggccgctgccacccttgtcaccttcaaggacgttttcgtggattttactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1309 - DNA, 213 bp, synthetic construct
  source 1..213: mol_type = other DNA
gatgccaagtccctgaagaagtggtctcggacccttgtgacttttaaggatgttttcgtcgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1310 - DNA, 213 bp, synthetic construct
  source 1..213: mol_type = other DNA
gatgctaagtccctgacggccgggggtggcacccttgttaccttcaaggacgtctttgtggatttcactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1311 - DNA, 213 bp, synthetic construct
  source 1..213: mol_type = other DNA
gatgatgattctctgacggcctggtcccggactcttgtcacctttaaggacgttttcgtggacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1312 - DNA, 213 bp, synthetic construct
  source 1..213: mol_type = other DNA
gacgccaagtccctgaccgcttggtctcggacgcttgtgacctttaaggatgttttcgtcgatttcactagggaagaatggaaactactagacacacaacaacaaatagtatacagaaacgtaatgctagaaaactacaaaaacctagtaagcctaggataccaactaacaaaaccagacgtaatactaagactagaaaaaggagaagaacca

SEQ ID NO: 1313 - DNA, 213 bp, synthetic construct
  source 1..213: mol_type = other DNA
gatgctaagtcccttaccgcctggtctcggacgctggtcaccttcaaggacgtgttcgttgattttactagggaagaatggaaactactagacacagcacaacaaatagtatacagaaacgtaatgctagaaaactacaaaaacctagtaagcctaggataccaactaacaaaaccagacgtaatactaagactagaaaaaggagaatactac

SEQ ID NO: 1314 - DNA, 213 bp, synthetic construct
  source 1..213: mol_type = other DNA
gatgctaagtcccttacggccgaggagaggactctggtcaccttcaaggacgtgttcgttgattttacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1315 - DNA, 213 bp, synthetic construct
  source 1..213: mol_type = other DNA
gatgccaagtctctgacggctcactccaggactcttgtcaccttcaaggatgtgttcgttgactttacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1316 - DNA, 213 bp, synthetic construct
  source 1..213: mol_type = other DNA
gatgccaagtcccttcgccggtggtctcgaactctggtcacctttaaggatgtgttcgttgacttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1317 - DNA, 213 bp, synthetic construct
  source 1..213: mol_type = other DNA
gatgctaagtctcttactgcctggtccagctcaagtgtgacctttaaggacgttttcgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1318 - DNA, 213 bp, synthetic construct
  source 1..213: mol_type = other DNA
gatgccaagtctcttaccgcttggtcccggactctggtcacgtttaaggacgttttcgtggatttcaccagggaagaatggaaactactagacacagcacaacaaatagtatacagaaacgtaatgctagaaaactacaaaaacctagtaagcctaggatggcaactaacaaaaccagacgtaatactaagactagaaaaaggagaagaacca

SEQ ID NO: 1319 - DNA, 213 bp, synthetic construct
  source 1..213: mol_type = other DNA
gattcatcttccctgacggcctggagtaggacccttgttaccttcaaggacgtctttgtggatttcactcgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1320 - DNA, 213 bp, synthetic construct
  source 1..213: mol_type = other DNA
gatgccaagtctctgaccgcttggtcccggactcttgtgacgttcaaggacgtctttgttgatttcaccagggaagaatggaaactactagacacagcacaaaaaaaagtatacagaaacgtaatgctagaaaactacaaaaacctagtaagcctaggataccaactaacaaaaccagacgtaatactaagactagaaaaaggagaagaacca

SEQ ID NO: 1321 - DNA, 213 bp, synthetic construct
  source 1..213: mol_type = other DNA
gacgctaagtccctgaccgccaagaagaagactcttgtcaccttcaaggatgttttcgtggattttacgagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1322 - DNA, 213 bp, synthetic construct
  source 1..213: mol_type = other DNA
gatgctaagtcccttaccgcctggtctgggggtggcgtgaccttcaaggatgttttcgtcgactttactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1323 - DNA, 213 bp, synthetic construct
  source 1..213: mol_type = other DNA
gatgccaagtcccttactgcttggtctgacgatgatgtgaccttcaaggatgttttcgtcgactttaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1324 - DNA, 213 bp, synthetic construct
  source 1..213: mol_type = other DNA
gatgctaagactaccacggcctggtccaggactcttgttacctttaaggatgtcttcgtggacttcacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1325 - DNA, 213 bp, synthetic construct
  source 1..213: mol_type = other DNA
gatgccaagtcccttactgcttggggcggtgggctggtgaccttcaaggacgtttttgtcgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1326 - DNA, 213 bp, synthetic construct
  source 1..213: mol_type = other DNA
gacgccaagtctctgactgcttggtcccgggagcttgtgacctttaaggatgtcttcgttgatttcaccagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1327 - DNA, 213 bp, synthetic construct
  source 1..213: mol_type = other DNA
gacgccaagtctgatgatgactggtcccggacccttgtcaccttcaaggatgttttcgtggattttactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1328 - DNA, 213 bp, synthetic construct
  source 1..213: mol_type = other DNA
gacgccaagtcacttagcagttcctctaggactctggtgacctttaaggatgtcttcgttgatttcacccgggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1329 - DNA, 213 bp, synthetic construct
  source 1..213: mol_type = other DNA
gacgctaagtcccttacggcctctagttcaaccctggtgaccttcaaggatgtcttcgttgattttactagggaagaatggaaactactagacacagcacaacaaatagtatacagaaacgtaatgctagaaaactacaaaaacctagtaagcctaggataccaactaacaaaaccagacgtaatactaagactagaaaaaggagaagaacca

SEQ ID NO: 1330 - protein, 4 aa, synthetic construct
RKKR

SEQ ID NO: 1331 - protein, 188 aa, synthetic construct
SPFSPVQLHQLRAQILAYKMLARGQPLPETLQLAVQGGGGGSGEGQSDERALLDQLHTLLSNTDATGLEEIDRALGIPELVNQGQALEPKQGGGGSGFVTLKDVGMDFTLGDWEQLGLEQGDTFWDTALDNCQDLFLLDPPGGGGSGHEKFPSDLDLDMFNGSLECDMESIIRSELMDADGLDFNFDS

SEQ ID NO: 1332 - protein, 145 aa, synthetic construct
EGQSDERALLDQLHTLLSNTDATGLEEIDRALGIPELVNQGQALEPKQGGGGSGHEKFPSDLDLDMFNGSLECDMESIIRSELMDADGLDFNFDSGGGGSGFVTLKDVGMDFTLGDWEQLGLEQGDTFWDTALDNCQDLFLLDPP

SEQ ID NO: 1333 - protein, 145 aa, synthetic construct
EGQSDERALLDQLHTLLSNTDATGLEEIDRALGIPELVNQGQALEPKQGGGGSGFVTLKDVGMDFTLGDWEQLGLEQGDTFWDTALDNCQDLFLLDPPGGGGSGHEKFPSDLDLDMFNGSLECDMESIIRSELMDADGLDFNFDS

SEQ ID NO: 1334 - protein, 188 aa, synthetic construct
EGQSDERALLDQLHTLLSNTDATGLEEIDRALGIPELVNQGQALEPKQGGGGSGSPFSPVQLHQLRAQILAYKMLARGQPLPETLQLAVQGGGGGSGFVTLKDVGMDFTLGDWEQLGLEQGDTFWDTALDNCQDLFLLDPPGGGGSGHEKFPSDLDLDMFNGSLECDMESIIRSELMDADGLDFNFDS

SEQ ID NO: 1335 - protein, 188 aa, synthetic construct
EGQSDERALLDQLHTLLSNTDATGLEEIDRALGIPELVNQGQALEPKQGGGGSGFVTLKDVGMDFTLGDWEQLGLEQGDTFWDTALDNCQDLFLLDPPGGGGSGSPFSPVQLHQLRAQILAYKMLARGQPLPETLQLAVQGGGGGSGHEKFPSDLDLDMFNGSLECDMESIIRSELMDADGLDFNFDS

SEQ ID NO: 1336 - protein, 188 aa, synthetic construct
EGQSDERALLDQLHTLLSNTDATGLEEIDRALGIPELVNQGQALEPKQGGGGSGFVTLKDVGMDFTLGDWEQLGLEQGDTFWDTALDNCQDLFLLDPPGGGGSGHEKFPSDLDLDMFNGSLECDMESIIRSELMDADGLDFNFDSGGGGSGSPFSPVQLHQLRAQILAYKMLARGQPLPETLQLAVQG

SEQ ID NO: 1337 - protein, 80 aa, synthetic construct
AFAAPRQRGKGEITPAAIQKMLDDNNHLIQCIMDSQNKGKTSECSQYQQMLHTNLVYLATIADSNQNMQSLLPAPPTQNM

SEQ ID NO: 1338 - protein, 145 aa, synthetic construct
HEKFPSDLDLDMFNGSLECDMESIIRSELMDADGLDFNFDSGGGGSGEGQSDERALLDQLHTLLSNTDATGLEEIDRALGIPELVNQGQALEPKQGGGGSGFVTLKDVGMDFTLGDWEQLGLEQGDTFWDTALDNCQDLFLLDPP

SEQ ID NO: 1339 - protein, 145 aa, synthetic construct
HEKFPSDLDLDMFNGSLECDMESIIRSELMDADGLDFNFDSGGGGSGFVTLKDVGMDFTLGDWEQLGLEQGDTFWDTALDNCQDLFLLDPPGGGGSGEGQSDERALLDQLHTLLSNTDATGLEEIDRALGIPELVNQGQALEPKQ

SEQ ID NO: 1340 - protein, 145 aa, synthetic construct
FVTLKDVGMDFTLGDWEQLGLEQGDTFWDTALDNCQDLFLLDPPGGGGSGEGQSDERALLDQLHTLLSNTDATGLEEIDRALGIPELVNQGQALEPKQGGGGSGHEKFPSDLDLDMFNGSLECDMESIIRSELMDADGLDFNFDS

SEQ ID NO: 1341 - protein, 145 aa, synthetic construct
FVTLKDVGMDFTLGDWEQLGLEQGDTFWDTALDNCQDLFLLDPPGGGGSGHEKFPSDLDLDMFNGSLECDMESIIRSELMDADGLDFNFDSGGGGSGEGQSDERALLDQLHTLLSNTDATGLEEIDRALGIPELVNQGQALEPKQ

SEQ ID NO: 1342 - protein, 98 aa, synthetic construct
EGQSDERALLDQLHTLLSNTDATGLEEIDRALGIPELVNQGQALEPKQGGGGSGFVTLKDVGMDFTLGDWEQLGLEQGDTFWDTALDNCQDLFLLDPP

SEQ ID NO: 1343 - protein, 95 aa, synthetic construct
EGQSDERALLDQLHTLLSNTDATGLEEIDRALGIPELVNQGQALEPKQGGGGSGHEKFPSDLDLDMFNGSLECDMESIIRSELMDADGLDFNFDS

SEQ ID NO: 1344 - protein, 91 aa, synthetic construct
FVTLKDVGMDFTLGDWEQLGLEQGDTFWDTALDNCQDLFLLDPPGGGGSGHEKFPSDLDLDMFNGSLECDMESIIRSELMDADGLDFNFDS

SEQ ID NO: 1345 - protein, 83 aa, synthetic construct
PRLKNVDRSTAQQLAVTVGNVTVIITDFKEKTGGGGSGIKEEPLDDYDGSSSSPENDDLFMMPRIVNVTSLATEGGLVDMGGS

SEQ ID NO: 1346 - protein, 44 aa, synthetic construct
SKKNSHKKTRPRLKNVDRSSAQHLEVTVGDLTVIITDFKEKTKS

SEQ ID NO: 1347 - protein, 45 aa, synthetic construct
IKEEPLDDYDGSSSSPENDDLFMMPRIVNVTSLATEGGLVDMGGS

SEQ ID NO: 1348 - protein, 80 aa, synthetic construct
ALRDFLLEAQPTDMRALQDFEEPDKLHIQMNDVITVIEGRAENYWWRGQNTRTLCVGPFPRNVVTSVAGLSAQDISQPLQ

SEQ ID NO: 1349 - protein, 80 aa, synthetic construct
SMELAKNKSKKRRNRTTFSTFQLEELEKVFQKTHYPDVYAREQLALRTDLTEARVQVWFQNRRAKWRKRERYGKIQEGRN

SEQ ID NO: 1350 - protein, 80 aa, synthetic construct
GAQVNMPADSFESPLTLAACGGHVELAALLIERGASLEEVNDEGYTPLMEAAREGHEEMVALLLGQGANINAQTEETQET

SEQ ID NO: 1351 - protein, 80 aa, synthetic construct
LPVPLDSAFEPAFLRKRNERERQRVRCVNEGYARLRDHLPRELADKRLSKVETLRAAIDYIKHLQELLERQAWGLEGAAG

SEQ ID NO: 1352 - protein, 80 aa, synthetic construct
KRHQEREKAARIIQLAVINFLAKQRLRKRVNAALVIQKYWRRVLAQRKLLMLKKEKLEKVQNKAASLIQGYWRRYSTRQR

SEQ ID NO: 1353 - protein, 80 aa, synthetic construct
KLTAVRTQAVICIQSYYRGFKVRKDIQNMHRAATLIQSFYRMHRAKVDYETKKTAIVVIQNYYRLYVRVKTERKNFLAVQ

SEQ ID NO: 1354 - protein, 80 aa, synthetic construct
IGEELAQLKEQRVHKTDLERVLEANDGSGMLDEDEEDLQRALALSRQEIDMEDEEADLRRAIQLSMQGSSRNISQDMTQT

SEQ ID NO: 1355 - protein, 80 aa, synthetic construct
EEEEEGLPMSYDEKRQLSLDINRLPGEKLGRVVHIIQSREPSLRDSNPDEIEIDFETLKPTTLRELERYVKSCLQKKQRK

SEQ ID NO: 1356 - protein, 80 aa, synthetic construct
EEEDKCKPMSYEEKRQLSLDINKLPGEKLGRVVHIIQSREPSLKNSNPDEIEIDFETLKPSTLRELERYVTSCLRKKRKP

SEQ ID NO: 1357 - protein, 80 aa, synthetic construct
NKKKVEEVLEEEEEEYVVEKVLDRRVVKGKVEYLLKWKGFSDEDNTWEPEENLDCPDLIAEFLQSQKTAHETDKSEGGKR

SEQ ID NO: 1358 - protein, 80 aa, synthetic construct; identical to SEQ ID NO: 508

SEQ ID NO: 1359 - protein, 80 aa, synthetic construct
AREKSAGKRGPDRGSPEYRQRRERNNIAVRKSRDKAKRRNQEMQQKLVELSAENEKLHQRVEQLTRDLAGLRQFFKQLPS

SEQ ID NO: 1360 - protein, 80 aa, synthetic construct
AFKADSLVEGTSSRYSMYNSVSQKLMAKMGFREGEGLGKYSQGRKDIVEASSQKGRRGLGLTLRGFDQELNVDWRDEPEP

SEQ ID NO: 1361 - protein, 80 aa, synthetic construct
ASEELQKDLEEVKVLLEKATRKRVRDALTAEKSKIETEIKNKMQQKSQKKAELLDNEKPAAVVAPITTGYTVKISNYGWD

SEQ ID NO: 1362 - protein, 80 aa, synthetic construct
PGENSKSRFPPQCYATQYQDYSLSSAHKVSGMASRLLGPSFESYLLPELTRYDCEVNVPVLGSSTLLQGGDLLRALDQAT

SEQ ID NO: 1363 - protein, 80 aa, synthetic construct
TAEGPQPPERKRRRRTAFTQFQLQELENFFDESQYPDVVARERLAARLNLTEDRVQVWFQNRRAKWKRNQRVLMLRNTAT

SEQ ID NO: 1364 - protein, 80 aa, synthetic construct
GKVEQLSPEEEEKRRIRRERNKMAAAKCRNRRRELTDTLQAETDQLEDEKSALQTEIANLLKEKEKLEFILAAHRPACKI

SEQ ID NO: 1365 - protein, 80 aa, synthetic construct
AGPPGLGGPRPVKRRGTANRKERRRTQSINSAFAELRECIPNVPADTKLSKIKTLRLATSYIAYLMDLLAKDDQNGEAEA

SEQ ID NO: 1366 - protein, 80 aa, synthetic construct
RKACQIFVRNLPFDFTWKMLKDKFNECGHVLYADIKMENGKSKGCGVVKFESPEVAERACRMMNGMKLSGREIDVRIDRN

SEQ ID NO: 1367 - protein, 80 aa, synthetic construct
QHPPDACAFRRGRKKRIPYSKGQLRELEREYAANKFITKDKRRKISAATSLSERQITIWFQNRRVKEKKVLAKVKNSATP

SEQ ID NO: 1368 - protein, 80 aa, synthetic construct
TTGNWLTAKSGRKKRCPYTKHQTLELEKEFLFNMYLTRERRLEISKTINLTDRQVKIWFQNRRMKLKKMNRENRIRELTS

SEQ ID NO: 1369 - protein, 80 aa, synthetic construct
SLAIARGRGKGPAAEEPLSLLDDMNHCYSRLRELVPGVPRGTQLSQVEILQRVIDYILDLQVVLAEPAPGPPDGPHLPIQ

SEQ ID NO: 1370 - protein, 80 aa, synthetic construct
GLITRLQARCRGYLVRQEFRSRMNFLKKQIPAITCIQSQWRGYKQKKAYQDRLAYLRSHKDEVVKIQSLARMHQARKRYR

SEQ ID NO: 1371 - protein, 80 aa, synthetic construct
NQRFIYGNQDLFATSQSKEFDPLGPLPPGWEKRTDSNGRVYFVNHNTRITQWEDPRSQGQLNEKPLPEGWEMRFTVDGIP

SEQ ID NO: 1372 - protein, 80 aa, synthetic construct
QPVKSELDEEEERRKRRREKNKVAAARCRNKKKERTEFLQRESERLELMNAELKTQIEELKQERQQLILMLNRHRPTCIV

SEQ ID NO: 1373 - protein, 80 aa, synthetic construct
VPSYNQTNSSMDFRNYMMRDETLEPLPKNWEMAYTDTGMIYFIDHNTKTTTWLDPRLCKKAKAPEDCEDGELPYGWEKIE

SEQ ID NO: 1374 - protein, 80 aa, synthetic construct
LQKNKQRLRNDPLNQNKGKPDLNTTLPIRQTASIFKQPVTKVTNHPSNKVKSDPQRMNEQPRQLFWEKRLQGLSASDVTE

SEQ ID NO: 1375 - protein, 80 aa, synthetic construct
GSILLDKDGKRKHTRPTFSGQQIFALEKTFEQTKYLAGPERARLAYSLGMTESQVKVWFQNRRTKWRKKHAAEMATAKKK

SEQ ID NO: 1376 - protein, 80 aa, synthetic construct
LPPGSTKRALPNNTSSSPQPKKKPLDGEYFTLQIRGRERFEMFRELNEALELKDAQAGKEPGGSRAHSSHLKSKKGQSTS

SEQ ID NO: 1377 - protein, 80 aa, synthetic construct
PSVEPPLSQETFSDLWKLLPENNVLSPLPSQAMDDLMLSPDDIEQWFTEDPGPDEAPRMPEAAPPVAPAPAAPTPAAPAP

SEQ ID NO: 1378 - protein, 80 aa, synthetic construct
NGLTTVALKETSTEALMKSSPEERERMIKQLKEELRLEEAKLVLLKKLRQSQIQKEATAQKPTGSVGSTVTTPPPLVRGT

SEQ ID NO: 1379 - protein, 80 aa, synthetic construct
KDIQHAGVPGEEPKCNICLGKTSGPLNEILICGKCGLGYHQQCHIPIAGSADQPLLTPWFCRRCIFALAVRKGGALKKGA

SEQ ID NO: 1380 - protein, 80 aa, synthetic construct
VSRDQAHLGPKYVGLWDFKSRTDEELSFRAGDVFHVARKEEQWWWATLLDEAGGAVAQGYVPHNYLAERETVESEPWFFG

SEQ ID NO: 1381 - protein, 80 aa, synthetic construct
ELARLNGTLREDDNRILRPWQSSETRSITNTTVCTKCGGAGHIASDCKFQRPGDPQSAQDKARMDKEYLSLMAELGEAPV

SEQ ID NO: 1382 - protein, 80 aa, synthetic construct
SSALTHAGAHLDLSAFSSWEELASLGLDRLKSALLALGLKCGGTLEERAQRLFSTKGKSLESLDTSLFAKNPKSKGTKRD

SEQ ID NO: 1383 - protein, 80 aa, synthetic construct
NWDKLVGEIKEEEKNEKLEGDAALNRLFQQIYSDGSDEVKRAMNKSFMESGGTVLSTNWSDVGKRKVEINPPDDMEWKKY

SEQ ID NO: 1384 - protein, 80 aa, synthetic construct
DNKKRLAYAIIQFLHDQLRHGGLSSDAQESLEVAIQCLETAFGVTVEDSDLALPQTLPEIFEAAATGKEMPQDLRSPART

SEQ ID NO: 1385 - protein, 80 aa, synthetic construct
AFLASVVDPRVASAAAKSALEEFSKMKEEVPTALVEAHVRKVEEAAKVTGKADPAFGLESSGIAGTTSDEPERIEESGND

SEQ ID NO: 1386 - protein, 80 aa, synthetic construct
KPSDHIKRPMNAFMVWSRGQRRKMAQENPKMHNSEISKRLGAEWKLLSEAEKRPYIDEAKRLRAQHMKEHPDYKYRPRRK

SEQ ID NO: 1387 - protein, 80 aa, synthetic construct
AGSKAPASAQSRGEKRTAHNAIEKRYRSSINDKIIELKDLVVGTEAKLNKSAVLRKAIDYIRFLQHSNQKLKQENLSLRT

SEQ ID NO: 1388 - protein, 80 aa, synthetic construct
KKKDFDTALKHYDKAKELDPTNMTYITNQAAVYFEKGDYNKCRELCEKAIEVGRENREDYRQIAKAYARIGNSYFKEEKY

SEQ ID NO: 1389 - protein, 80 aa, synthetic construct
AEIKAARERAIVPLEARMKQFKDMLLERGVSAFSTWEKELHKIVFDPRYLLLNPKERKQVFDQYVKTRAEEERREKKNKI

SEQ ID NO: 1390 - protein, 80 aa, synthetic construct
TKEIDREREQHKREEAIQNFKALLSDMVRSSDVSWSDTRRTLRKDHRWESGSLLEREEKEKLFNEHIEALTKKKREHFRQ

SEQ ID NO: 1391 - protein, 80 aa, synthetic construct
ANVTQLVKQLRTERPSGPGGEMGVCEKHREPLKLYCEEDQMPICVVCDRSREHRGHSVLPLEEAVEGFKEQIQNQLDHLK

SEQ ID NO: 1392 - protein, 80 aa, synthetic construct
LKDKFDGRPTKPPPNSYSLYCAELMANMKDVPSTERMVLCSQQWKLLSQKEKDAYHKKCDQKKKDYEVELLRFLESLPEE

SEQ ID NO: 1393 - protein, 80 aa, synthetic construct
AAKSRQPDGCMDPVRQQSGNANTETLPSGWEQRKDPHGRTYYVDHNTRTTTWERPQPLPPGWERRVDDRRRVYYVDHNTR

SEQ ID NO: 1394 - protein, 80 aa, synthetic construct
TPAEGEEPSTSGTQQLPAAAQAPDALPAGWEQRELPNGRVYYVDHNTKTTTWERPLPPGWEKRTDPRGRFYYVDHNTRTT

SEQ ID NO: 1395 - protein, 80 aa, synthetic construct
GGGSGNDNNQAATKSPRKAAAAAARLNRLKKKEYVMGLESRVRGLAAENQELRAENRELGKRVQALQEESRYLRAVLANE

SEQ ID NO: 1396 - protein, 80 aa, synthetic construct
CKWIDPDELAGLPPPPPPPPPPPPPPPAGGAKPCSKTFGTMHELVNHVTVEHVGGPEQSSHVCFWEDCPREGKPFKAKYK

SEQ ID NO: 1397 - protein, 80 aa, synthetic construct
SQDSVTFADVAVNFTKEEWTLLDPAQRNLYRDVMLENSRNLAFIDWATPCKTKDATPQPDILPKRTFPEANRVCLTSISS

SEQ ID NO: 1398 - protein, 80 aa, synthetic construct
KPFECKECGKAFSWGSSLVKHERVHTGEKSHECKECGKTFCSGYQLTRHQVFHTGEKPYECKECGKAFNCGSSLVQHERI

SEQ ID NO: 1399 - protein, 80 aa, synthetic construct
GSQGNLPQADITLMSQAQESVTFEDVAVNFTNREWQCLTYAQRHLYKDVMLENYGNMVSLGFPFPKPPLISHLEREVDPC

SEQ ID NO: 1400 - protein, 80 aa, synthetic construct
ELVTFRDVAIEFSPEEWKCLDPAQQNLYRDVMLENYRNLVSLGFVISNPDLVTCLEQIKEPCNLKIHETAAKPPAICSPF

SEQ ID NO: 1401 - protein, 80 aa, synthetic construct
DSVSFEDVAVAFTQEEWALLDPSQKNLYRDVMQEIFRNLASVGNKSEDQNIQDDFKNPGRNLSSHVVERLFEIKEGSQYG

SEQ ID NO: 1402 - protein, 20 aa, synthetic construct
DAKSLTAWSRTLVTFKDVFV

SEQ ID NO: 1403 - protein, 20 aa, synthetic construct
AAKSLTAWSRTLVTFKDVFV

SEQ ID NO: 1404 - protein, 20 aa, synthetic construct
CAKSLTAWSRTLVTFKDVFV

SEQ ID NO: 1405 - protein, 20 aa, synthetic construct
DCKSLTAWSRTLVTFKDVFV

SEQ ID NO: 1406 - protein, 20 aa, synthetic construct
DDKSLTAWSRTLVTFKDVFV

SEQ ID NO: 1407 - protein, 20 aa, synthetic construct
KKKSLTAWSRTLVTFKDVFV

SEQ ID NO: 1408 - protein, 20 aa, synthetic construct
LLKSLTAWSRTLVTFKDVFV

SEQ ID NO: 1409 - protein, 20 aa, synthetic construct
GATGCTAAATCTTTAACTGC

SEQ ID NO: 1410 - protein, 20 aa, synthetic construct
GCCGCCAAGAGCCTGACCGC

SEQ ID NO: 1411 - protein, 20 aa, synthetic construct
TGCGCCAAGTCGCTGACCGC

SEQ ID NO: 1412 - protein, 20 aa, synthetic construct
GATTGCAAATCTCTTACTGC

SEQ ID NO: 1413 - protein, 20 aa, synthetic construct
GACGACAAGAGCCTGACCGC

SEQ ID NO: 1414 - protein, 20 aa, synthetic construct
AAAAAAAAAAGCCTGACCGC

SEQ ID NO: 1415 - protein, 20 aa, synthetic construct
CTGCTGAAAAGCCTGACCGC

SEQ ID NO: 1416 - protein, 80 aa, synthetic construct [sequence omitted]

SEQ ID NO: 1417 - protein, 80 aa, synthetic construct; identical to SEQ ID NO: 1367

SEQ ID NO: 1418 - protein, 80 aa, synthetic construct
LQPEVSSYRRGRKKRVPYTKVQLKELEKEYAASKFITKEKRRRISATTNLSERQVTIWFQNRRVKEKKVVSKSKAPHLHS

SEQ ID NO: 1419 - protein, 80 aa, synthetic construct
DKAGGSSGQRTRKKRCPYTKYQIRELEREFFFSVYINKEKRLQLSRMLNLTDRQVKIWFQNRRMKEKKINRDRLQYYSAN

SEQ ID NO: 1420 - protein, 80 aa, synthetic construct
AKGAAPNAPRTRKKRCPYSKFQIRELEREFFFNVYINKEKRLQLSRMLNLTDRQVKIWFQNRRMKEKKLSRDRLQYFSGN

SEQ ID NO: 1421 - protein, 80 aa, synthetic construct; identical to SEQ ID NO: 1368

SEQ ID NO: 1422 - protein, 80 aa, synthetic construct
DGLPWGAAPGRARKKRKPYTKQQIAELENEFLVNEFINRQKRKELSNRLNLSDQQVKIWFQNRRMKKKRVVLREQALALY

SEQ ID NO: 1423 - protein, 80 aa, synthetic construct
NAANWLTAKSGRKKRCPYTKHQTLELEKEFLFNMYLTRERRLEISRSVHLTDRQVKIWFQNRRMKLKKMNRENRIRELTA

SEQ ID NO: 1424 - protein, 80 aa, synthetic construct
SGAPWYPINSRSRKKRKPYSKLQLAELEGEFLVNEFITRQRRRELSDRLNLSDQQVKIWFQNRRMKKKRLLLREQALSFF

SEQ ID NO: 1425 - protein, 80 aa, synthetic construct
DQTNPSANWLHARSSRKKRCPYTKYQTLELEKEFLFNMYLTRDRRHEVARLLNLSERQVKIWFQNRRMKMKKMNKEQGKE

SEQ ID NO: 1426 - protein, 80 aa, synthetic construct
PNNPAANWLHARSTRKKRCPYTKHQTLELEKEFLFNMYLTRDRRYEVARLLNLTERQVKIWFQNRRMKMKKINKDRAKDE

SEQ ID NO: 1427 - protein, 80 aa, synthetic construct
GDKSPPGQASSKRARTAYTSAQLVELEKEFHFNRYLCRPRRVEMANLLNLTERQIKIWFQNRRMKYKKDQKGKGMLTSSG

SEQ ID NO: 1428 - protein, 80 aa, synthetic construct
CEDKSPPGPASKRVRTAYTSAQLVELEKEFHFNRYLCRPRRVEMANLLNLTERQIKIWFQNRRMKYKKDQKAKGILHSPA

SEQ ID NO: 1429 - protein, 79 aa, synthetic construct
GDKSPPGSAASKRARTAYTSAQLVELEKEFHFNRYLCRPRRVEMANLLNLSEQIKIWFQNRRMKYKKDQKAKGLASSSG

SEQ ID NO: 1430 - protein, 80 aa, synthetic construct
PWMRPQAAPGRRRGRQTYSRFQTLELEKEFLFNPYLTRKRRIEVSHALALTERQVKIWFQNRRMKWKKENNKDKFPVSRQ

SEQ ID NO: 1431 - protein, 80 aa, synthetic construct
SVNPNYTGGEPKRSRTAYTRQQVLELEKEFHFNRYLTRRRRIEIAHTLCLSERQIKIWFQNRRMKWKKDHKLPNTKGRSS

SEQ ID NO: 1432 - protein, 79 aa, synthetic construct
HISHDMTGPDGKRARTAYTRYQTLELEKEFHFNRYLTRRRRIEIAHALCLSERQIKWFQNRRMKWKKDNKLKSMSLATA

SEQ ID NO: 1433 - protein, 79 aa, synthetic construct
LEIADGSGGSRRLRTAYTNTQLLELEKEFHFNKYLCRPRRVEIAALLDLTERQVKVWFQNRRMKHKRQTQCKENQNSEQ

SEQ ID NO: 1434 - protein, 80 aa, synthetic construct
TVNPNYNGGEPKRSRTAYTRQQVLELEKEFHYNRYLTRRRRIEIAHSLCLSERQIKIWFQNRRMKWKKDHRLPNTKVRSA

SEQ ID NO: 1435 - protein, 79 aa, synthetic construct
SCNSSFGPGSRRGRQTYTRYQTLELEKEFHYNRYLTRRRRIEIAHALCLTREQIKIWFQNRRMKWKKESKLLSASQLSA

SEQ ID NO: 1436 - protein, 80 aa, synthetic construct
AVNPSYNGGEPKRSRTAYTRQQVLELEKEFHFNRYLTRRRRIEIAHTLCLSERQVKIWFQNRRMKWKKDHKLPNTKMRSS

SEQ ID NO: 1437 - protein, 80 aa, synthetic construct
KVGEYGYLGQPNAVRTNFTTKQLTELEKEFHFNKYLTRARRVEIAASLQLNETQVKIWFQNRRMKQKKREKEGLLPISPA

SEQ ID NO: 1438 - protein, 80 aa, synthetic construct
AKVSEPGLGSPSGLRTNFTTRQLTELEKEFHFNKYLSRARRVEIAATLELNETQVKIWFQNRRMKQKKREREEGRVPPAP

SEQ ID NO: 1439 - protein, 79 aa, synthetic construct
KLAEYGAASPSSAIRTNFSTKQLTELEKEFHFNKYLTRARRIEIANCLHLNDTQVKIWFQRRMKQKKREREGLLATAIP

SEQ ID NO: 1440 - protein, 71 aa, synthetic construct
DAKSLTAWSRTLVTFKDVFVDFTREEWKLLDTAQQIVYRNVMLENYKNLVSLGYQLTKPDVILRLEKGEEP

SEQ ID NO: 1441 - protein, 64 aa, synthetic construct
GSSGSSGVTYDDVHMNFTEEEWDLLDSSQKRLYEEVMLETYQNLTDIGYNWQDHHIEESGPSSG

SEQ ID NO: 1442 - protein, 61 aa, synthetic construct
EMLSFRDVAVDFSAEEWECLEPAQWNLYRDVMLENYSHLVFLGLAFSKPYLVTFLEQRQEP

SEQ ID NO: 1443 - protein, 20 aa, synthetic construct
GAVLMIFYWKRHDESTCNQP

SEQ ID NO: 1444 - protein, 50 aa, synthetic construct
EISECLIASSFEDDSRVASPLDQNGSFNVVIKEEPLDDYDYELGECPEGV